(19) United States
(12) Patent Application Publication
(10) Pub. No.: US 2005/0107870 A1
(43) Pub. Date: May 19, 2005

(54) Medical device with multiple coating layers

(76) Inventors: Xingwu Wang, Wellsville, NY (US); Howard J. Greenwald, Rochester, NY (US)

Correspondence Address: HOWARD J. GREENWALD P.C., 349 W. COMMERCIAL STREET SUITE 2490, EAST ROCHESTER, NY, 14445-2408, US

(21) Appl. No.: 10/923,579

(22) Filed: Aug. 20, 2004

Related U.S. Application Data
(63) Continuation-in-part of application No. 10/914,691, filed on Aug. 9, 2004.
(63) Continuation-in-part of application No. 10/887,521, filed on Jul. 7, 2004.
(63) Continuation-in-part of application No. 10/867,517, filed on Jun. 14, 2004.
(63) Continuation-in-part of application No. 10/810,916, filed on Mar. 26, 2004, now Pat. No. 6,846,985.
(63) Continuation-in-part of application No. 10/808,618, filed on Mar. 24, 2004.
(63) Continuation-in-part of application No. 10/786,198, filed on Feb. 25, 2004.
(63) Continuation-in-part of application No. 10/780,045, filed on Feb. 17, 2004.
(63) Continuation-in-part of application No. 10/747,472, filed on Dec. 29, 2003.
(63) Continuation-in-part of application No. 10/744,543, filed on Dec. 22, 2003.
(63) Continuation-in-part of application No. 10/442,420, filed on May 21, 2003.
(63) Continuation-in-part of application No. 10/409,505, filed on Apr. 8, 2003, now Pat. No. 6,815,609.

(51) Int. Cl. A61F 2/06

(52) U.S. Cl. 623/1.44

(57) ABSTRACT

An implantable medical device that contains two coating layers disposed above at least one of its surfaces. The first coating layer contains a biologically active material; and the second coating layer contains a polymeric material and nanomagnetic material disposed on the first coating layer; the second coating layer is substantially free of the biologically active material. The nanomagentic material has a saturation magentization of from about 2 to about 3000 electromagnetic units per cubic centimeter, and it contains nanomagnetic particleswith an average particle size of less than about 100 nanometers; the average coherence length between adjacent nanomagnetic particles is less than 100 nanometers.

### CROSS-REFERENCE TO RELATED PATENT APPLICATION

This patent application is a continuation in part of each of applicants' copending patent application Ser. No. 10/914,691 (filed on Aug. 8, 2004), Ser. No. 10/887,521 (filed on Jul. 7, 2004), Ser. No. 10,867,517 (filed on Jun. 14, 2004), Ser. No. 10/810,916 (filed on Mar. 26, 2004), Ser. No. 10/808,618 (filed on Mar. 24, 2004), Ser. No. 10/786,198 (filed on Feb. 25, 2004), Ser. No. 10/780,045 (filed on Feb. 17, 2004), Ser. No. 10/747,472 (filed on Dec. 29, 2003), Ser. No. 10/744,543 (fled on Dec. 22, 2003), Ser. No. 10/442,420 (filed on May 21, 2003), and Ser. No. 10/409,505 (flied on Apr. 8, 2003). The entire disclosure of each of these patent applications is hereby incorporated by reference into this specification.

### FIELD OF THE INVENTION

A medical device that contains at least two coating layers disposed on its surface; the first coating layer contains a biologically active material; and the second coating layer contains a polymeric material and particles of naonmagneic material.

### BACKGROUND

Published U.S. patent application No. 2004/0030379 describes a “medical device and system capable of providing on-demand delivery of biologically active material to a body lumen patient . . . .” As is disclosed on page 1 of this published patent application, “In order to treat a variety of medical conditions, insertable or implantable medical devices having a coating for release of a biologically active material have been used. For example, various types of drug-coated stents have been used for localized delivery of drugs to a body lumen. See U.S. Pat. No. 6,099,562 to Ding et al. Such stents have been used to prevent, inter alia, the occurrence of restenosis after balloon angioplasty. However, delivery of the biologically active material to the body tissue immediately after insertion or implantation of the stent may not be needed or desired. For instance, it may be more desirable to wait until restenosis occurs or begins to occur in a body lumen that has been stented with a drug-coated stent before the drug is released. Therefore, there is a need for implantable medical devices that can provide on-demand delivery of biologically active materials when such materials are required by the patient after implantation of the medical device. Also needed is a non-invasive method to facilitate or modulate the delivery of the biologically active material from the medical device after implantationof a heart valve which could be inserted with the patients only slightly sedated, locally anesthetized, and released from the hospital quickly (within a day) after a procedure and would allow the in situ magnetic resonance imaging of stents, has long been sought but yet equally as long eluded those skilled in the art” (see paragraph 0003).

The solution to this problem provided by published U.S. patent application No. 2004/0030379 is disclosed in claim 1 thereof, which describes: “1. A medical device that is insertable into the body of a patient comprising: (a) a surface; (b) a first coating layer comprising a biologically active material disposed on at least a portion of the surface; and (c) a second coating layer comprising a polymeric material and magnetic particles disposed on the first coating layer, wherein the second coating layer is substantially free of the biologically active material.” The “magnetic particles” used in such medical device may be either “iron oxide particles or magnetic silica particles” (see claim 7 of US 2004/0030379.

The device described in published U.S. patent No. 2004/00303779 is an improvement over the prior art devices, but it still suffers from the imageability problems described in published U.S. patent application No. 2004/0093075. In this latter published patent application, it is dislosed that: “In the medical field, magnetic resonance imaging (MRI) is used to non-invasively produce medical information. The patient is positioned in an aperture of a large annular magnet, and the magnet produces a strong and static magnetic field, which forces hydrogen and other chemical elements in the patient's body into alignment with the static field. A series of radio frequency (RF) pulses are applied orthogonally to the static magnetic field at the resonant frequency of one of the chemical elements, such as hydrogen in the water in the patient's body. The RF pulses force the spin of protons of chemical elements, such as hydrogen, from their magnetically aligned positions and cause the electrons to precess. This precession is sensed to produce electromagnetic signals that are used to create images of the patient's body. In order to create an image of a plane of patient cross-section, pulsed magnetic fields are superimposed on the high strength static magnetic field.”

Published U.S. patent application No. 2004/0093075 also discloses that “While researching heart problems, it was found that all the currently used metal stents distorted the magnetic resonance images of blood vessels. As a result, it was impossible to study the blood flow in the stents and the area directly around the stents for determining tissue response to different stents in the heart region.

The solution to this problem provided by U.S. published patent application No. 2004/0093075 is described in claim 1 thereof, which discloses “1. A valve device comprising an elongate, self-expanding, medical stent for a bodily lumen, which stent has a radially-compressed delivery configuration and a radially-expanded deployed configuration, the stent defining in said deployed configuration a stent lumen for flow of a bodily fluid lengthwise with respect to the stent within the bodily lumen and lengthwise within the bodily lumen, the stent supporting a valve leaflet which can move, in the deployed configuration tof the stent, between an open configuration to allow fluid flow along said stent lumen in one direction and a closed configuration in which the leaflet resists fluid flow along the stent lumen in a direction opposite to said one direction; characterised in that: said leaflet has a free periphery unattached to the stent and a resilient wire which extends around a substantial part of the length of said free periphery whereby, upon self-expansion of the stent from the delivery to the deployed configuration, the wire brings the leaflet into a disposition in which the leaflet extends across the stent lumen to an extent sufficient to permit fluid pressure differentials across the ends of the stent lumen to move the leaflet between its open and closed configurations.” This solution is not broadly applicable to many prior art stents, and to other assemblies. Although the applicant of US 2004/0093075 claims that the stents depicted in his FIGS. 11, 12, 13, and 14 have improved imageability, there is no claim made of a process for rendering other stents (and assemblies) with different configurations more imageable.

It is an object of this invention to provide such a medical device that has improved drug delivery capabilities and also has improved MRI imageability.

### SUMMARY OF THE INVENTION

In accordance with this invention, there is provided a medical device that contains at least two coating layers disposed on its surface. The first coating layer contains a biologically active material; and the second coating layer contains a polymeric material and particles of naonmagneic material. In one embodiment, the assembly has a magnetic susceptibility within the range of plus or minus 1×10⁻³ centimeter-gram-seconds

### BRIEF DESCRIPTION OF THE DRAWINGS

The above noted and other features of the invention will be better understood from the following drawings, and the accompanying description of them in the specification, wherein like numerals refer to like elements, and wherein:

FIG. 1 is a schematic diagram of one preferred seed assembly of the invention;

FIG. 1A is a schematic diagram of another preferred seed assembly of the invention;

FIG. 2 is a schematic illustration of one process of the invention that may be used to make nanomagnetic material;

FIG. 2A is a schematic illustration of a process that may be used to make and collect nanomagnetic particles;

FIG. 3 is a flow diagram of another process that may be used to make the nanomagnetic compositions of this invention;

FIG. 3A is a graph of the magnetic order of a nanomagnetic material plotted versus its temperature;

FIG. 4 is a phase diagram showing the phases in various nanomagnetic materials comprised of moieties A, B, and C;

FIGS. 4A and 4B illustrate how the magnetic order of the nanomagnetic particles of this invention is destroyed at a temperature in excess of the phase transition temperature;

FIG. 5 is a schematic representation of what occurs when an electromagnetic field is contacted with a nanomagentic material;

FIG. 5A illustrates the coherence length of the nanomagnetic particles of this invention;

FIG. 6 is a schematic sectional view of a shielded conductor assembly that is comprised of a conductor and, disposed around such conductor, a film of nanomagnetic material;

FIGS. 7A through 7E are schematic representations of other shielded conductor assemblies that are similar to the assembly of FIG. 6;

FIG. 8 is a schematic representation of a depositon system for the preparation of aluminum nitride materials;

FIG. 9 is a schematic, partial sectional illustration of a coated substrate that, in the preferred embodiment illustrated, is comprised of a coating disposed upon a stent;

FIG. 9A is a schematic illustration of a coated substrate that is similar to the coated substrate of FIG. 9 but differs therefrom in that it contains two layers of dielectric material;

FIG. 10 is a schematic view of a typical stent that is comprised of wire mesh constructed in such a manner as to define a multiplicity of openings;

FIG. 11 is a graph of the magnetization of an object (such as an uncoated stent, or a coated stent) when subjected to an electromagnetic filed, such as an MRI field,

FIG. 11A is a graph of the magnetization of a composition comprised of species with different magnetic suspceptibilities when subjected to an electromagnetic field, such as an MRI field;

FIG. 12 is a graph of the reactance of an object (such as an uncoated stent, or a coated stent) when subjected to an electromagnetic filed, such as an MRI field;

FIG. 13 is a graph of the image clarity of an object (such as an uncoated stent, or a coated stent) when subjected to an electromagnetic filed, such as an MRI field;

FIG. 14 is a phase diagram of a material that is comprised of moieties A, B, and C;

FIG. 15 is a schematic view of a coated substrate comprised of a substrate and a multiplicity of nanoelectrical particles;

FIGS. 16A and 16B illustrate the morphological density and the surface roughness of a coating on a substrate;

FIG. 17A is a schematic representation of a stent comprised of plaque disposed inside the inside wall;

FIG. 17B illustrates three images produced from the imaging of the stent of FIG. 17A, depending upon the orientation of such stent in relation to the MRI imaging apparatus reference line;

FIG. 17C illustrates three images obtained from the imaging of the stent of FIG. 17A when the stent has the nanomagnetic coating of this invention disposed about it;

FIGS. 18A and 18B illustrate a hydrophobic coating and a hydrophilic coating, respectively, that may be produced by the process of this invention;

FIG. 19 illustrates a coating disposed on a substrate in which the particles in their coating have diffused into the substrate to form a interfacial diffusion layer;

FIG. 20 is a sectional schematic view of a coated substrate comprised of a substrate and, bonded thereto, a layer of nano-sized particles;

FIG. 20A is a partial sectional view of an indentation within a coating that, in turn, is coated with a multiplicity of receptors;

FIG. 20B is a schematic of an electromagnetic coil set aligned to an axis and which in combination create a magnetic standing wave;

FIG. 20C is a three-dimensional schematic showing the use of three sets of magnetic coils arranged orthogonally;

FIG. 21 is a schematic illustration of one process for preparing a coating with morphological indentations;

FIG. 22 is a schematic illustration of a drug molecule disposed inside of a indentation;

FIG. 23 is a schematic illustration of one preferred process for administering a drug into the arm of a patient near a stent via an injector;

FIG. 24 is a schematic illustration of a preferred binding process of the invention;

FIG. 25 is a schematic view of a preferred coated stent of the invention;

FIG. 26 is a graph of a typical response of a magnetic drug particle to an applied electromagnetic field;

FIGS. 27A and 27B illustrate the effect of applied fileds upon a nanomagnetic and upon magnetic drug particles;

FIG. 28 is graph of a preferred nanomagnetic material and its response to an applied electromagnetic field, in which the applied field is applied against the magnetic moment of the nanomagnetic material;

FIG. 29 illustrates the forces acting upon a magnetic drug particle as it approaches nanomagnetic material;

FIG. 30 illustrates the situation that occurs after the drug particles have migrated into the layer of polymeric material and when one desires to release such drug particles;

FIG. 31 illustrates the situation that occurs after the drug particles have migrated into the layer of polymeric material but when no external electromagnetic field is imposed:

FIG. 32 is a partial view of a coated container over which is disposed a layer 5002 of material which changes its dimensions in response to an applied magnetic field;

FIG. 33 is a partial view of magnetostrictive magnetostrictive material prior to the time an orifice has been created in it;

FIG. 34 is a schematic illustration of a magnetostrictive material bounded by nanomagnetic material;

FIG. 35 is a schematic illustration of a preferred implantable device of this invention with improved MRI imageability;

FIG. 36 is a sectional view of a component of a preferred stent assembly;

FIG. 37 is a sectional view of another preferred medical device of the invention;

FIG. 38 is a sectional view of one a strut that is part of the medical device of FIG. 37;

FIG. 39 is a schematic illustration of the forces that exist between a coating on the strut of FIG. 38 and a magnetic particle disposed near such strut;

FIG. 40 is a flow diagram of a process for preparing one preferred stent of the invention;

FIG. 41 is a schematic illustration of a process for magnetizing a medical device;

FIG. 42 is a graph of an exponentially decaying magnetic field that can be used to demagnetize a magnetized device;

FIG. 43 is a schematic sectional veiwo of a drug-eluting medical device comprised of a layer of nanomagnetic material and a layer of giant magnetoresistive material;

FIG. 44 is a schematic illustration of a means for measuring surface eddy currents on a device;

FIG. 45 is a schematic illustration of the forces acting on the medical device of FIG. 43 when it is subjected toa magnetic resonance imaging (MRI) field; and

FIG. 46 is a partial sectional view of another preferred stent of this invention;

FIG. 47 is a cross-sectional view of another preferred stent of this invention;

FIG. 48A is a cross-sectional view of a coated strut 9020 of the stent of FIG. 47;

FIG. 48B shows the effect on the coated strut 9020 when a patient is exposed to an electromagnetic field 9090;

FIG. 49A is a cross-sectional view of another coated strut 9021;

FIG. 49B shows the effect on the coated strut 9021 when a patient is exposed to an electromagnetic field 9090;

FIG. 50A is a cross-sectional view of another coated strut 9023;

FIG. 50B shows the effect on the coated strut 9023 when a patient is exposed to an electromagnetic field 9090; and

FIG. 51 is a cross-sectional view of a coated strut 9027.

F

### DESCRIPTION OF THE PREFERRED EMBODIMENT

In the first part of this specification, a preferred seed assembly will be described. In the second part of this specification, a process for making an implanted medical device more imageable when subjected to magnetic resonance imaging (MRI) radiation will be described. Thereafter, in the third part of this specification, ana medical device with improved MRI imageability and drug delivery capabilities will be disclosed.

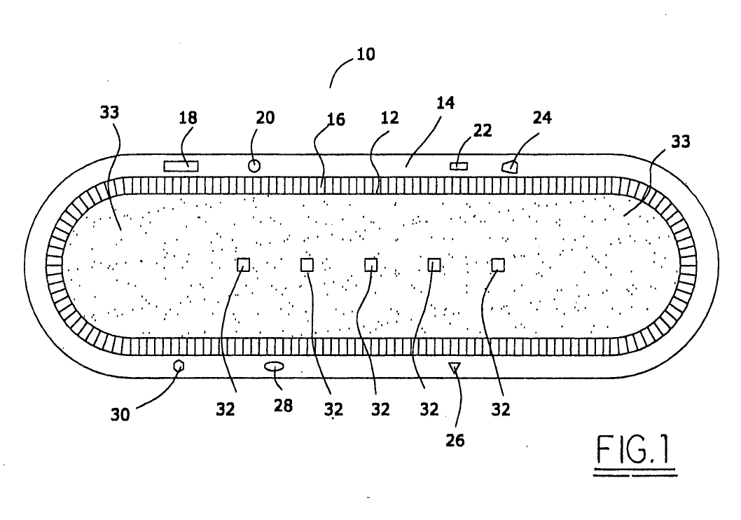

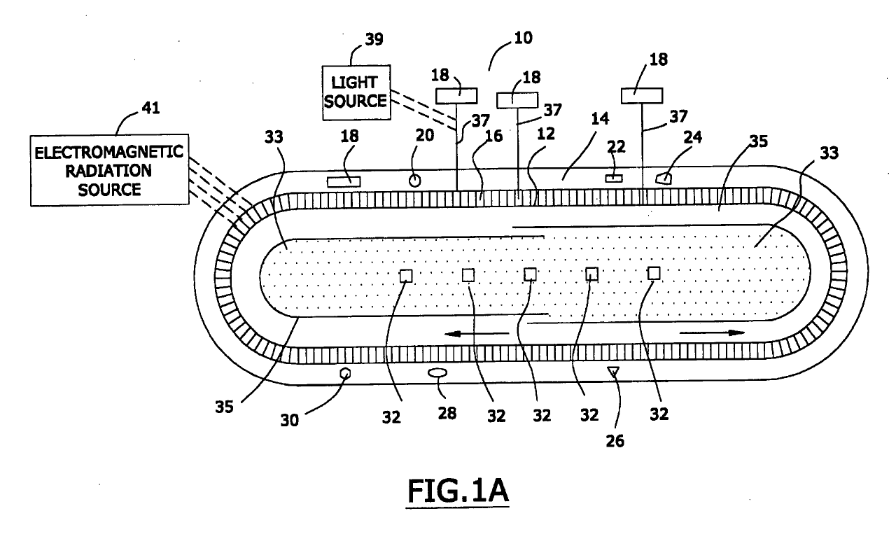

FIG. 1 is a schematic diagram of a preferred seed assembly 10 of this invention. Referring to FIG. 1, and to the preferred embodiment depicted therein, it will be seen that assembly 10 is comprised of a sealed container 12 comprised of a multiplicity of radioactive particles 33.

The sealed container 12 may be any of the containers conventionally used in brachytherapy.

Thus, e.g., one may use as container 12 an ampulla comprised of several compartments, as is described in U.S. Pat. No. 1,626,338; the entire disclosure of this United States patent is hereby incorporated by reference into this specification. In the ampulla of this patent, materials from different compartments communicate with each other to form “radium emissions.”

Thus, e.g., and referring to U.S. Pat. 2,269,458 (the entire disclosure of which is hereby incorporated by reference into this specification), one may use as container 12 “A capsule for containing a radioactive substance comprising a member having a socket therein for containing said substance and another member for closing the socket, one of said members being constructed of a magnetizable metal.” In one embodiment, the capsule is preferably made of a “magnetizable metal” and of a material that is permeable to the rays emitting from the radioactive material. “Duralumin” is described as being one material that is so permeable.

Thus, e.g., and referring to U.S. Pat. 2,959,166 (the entire disclosure of which is hereby incorporated by reference into this specification), one may use as container 12 “A radioactive material applicator, comprising, a supporting frame; means for attaching the frame to bone structure of a patient so as to be positioned in the pelvis of the patient; a plurality of radioactive material supports carried by the frame; and means for mounting radioactive material on the supports.” As was disclosed in column 6 of this patent, “There are several different kinds of radioactive material which may be used in the treatment of cancer. The most common type used is radium chloride, usually referred to as ‘radium.’ Radium chloride is in granular form, and is sealed in small cylinders of varying lengths, called ‘cells.’ . . . Another type of radioactive material which may be employed . . . is radioactive cobalt, which may be in the form of bars, sheets, or wires. Another form of radioactive material . . . is radioactive cesium-147, which is a fission product secured from atomic energy plants. This product is in powder form and may be sealed in small cylinders of varying lengths. A still further form of radioactive material usuable with my applicator is radioactive gold-198 . . . .” The radioactive materials of this United States patent may be used as radioactive material 33 (see FIG. 1).

Thus, e.g., and referring to U.S. Pat. No. 3,060,924 (the entire disclosure of which is hereby incorporated by reference into this specification) one may use as container 12 an “Apparatus for applying radioactive materials to a body cavity having anterior and posterior portions with a restricted passage therebetween, said apparatus comprising a shank having a handle and a stock portion, a plurality of resiliently flexible arms . . . , a plurality of pods for containing radioactive material . . . .” As is disclosed in column 3 of the patent, “ . . . the pod comprises a cylindrical casing 26 of a suitable material which will pass rays from radio-active material and which closing is closed at its upper end 27 and open at its lower end. The lower end portion of casing 26 is threaded to receive a cap 28 . . . .”

Thus, e.g., and referring to U.S. Pat. No. 3,351,049 (the entire disclosure of which is hereby incorporated by reference into this specification), one may use as container 12 “A radioactive seed . . . comprising a sealed container having an elongate cavity therein, and constructed with walls of substantially uniform thickness, a therapeutic amount of soft X-ray emanating radioisotope disposed within said cavity, said soft X-ray emanating isotope having a characteristic radiation substantially all of which lies between about 20 kev. and 100 kev . . . and means disposed within said cavity for maintaining said radioisotope in a substantially uniform distribution . . . .” It is disclosed in this patent (at column 2 thereof) that ““This invention is predicated upon the observation that there is a class of radioactive isotopes which characteristically emit a radiation principally limited to low energy X-rays . . . . These isotopes are unique in that their half-lives are sufficiently short that they decay predictably to a negligible output level and therefore can be left permanently and indefinitely implanted . . . .” The radioactive isotopes described in this patent may be used as radioactive material 33.

Thus, e.g., U.S. Pat. No. 3,750,653 discloses “A capsule adapted to be inserted in and retained by the uterus, comprising an elongated and enlarged bulbous body portion with a cavity therein, said cavity being disposed generally longitudinally within said body portion and having a diameter sufficient to accommodate a source of radioactive material therein, a thin-walled narrow tube connected to said body portion and arranged coaxially with said cavity so as to permit insertion of a radioactive source into said cavity through said tube, the outside diameter of said tube being not greater than 2 mm. so as to permit said capsule to be retained within and tolerated by the uterus with said tube projecting through the cervical so that said source may be inserted into the cavity after the capsule is positioned in the uterus.” In column 1 of this patent, the patentee also discloses “Hyman applicators” that are “ . . . metal cylinders about 8 mm. in diameter and 2 cm. long containing 5 to 10 milligrams of radium in each.” By comparison, the capsule of U.S. Pat. No. 3,750,653 comprised a thin-walled narrow tube whose outside diameter was no greater than about 2 millimeters in diameter. In columns 2 and 3 of this patent, it is disclosed that: “Extremely important to the invention is the fact that the outside diameter of the thin-walled tube is preferably no greater than 2 mm. Because of the small diameter the tubes can easily be inserted and retained by any portion of the human body. Such miniaturization was technically impossible until just recently with the development of radioactive isotopes with a specific activity higher than that of radium. Now very minute portions of radioactive isotopes such as iridium-192, cesium-137 and cobalt-60 emit sufficient radiation for the treatment of tumors.” Both the capsules described in this patent and the radioactive material described in this patent may be used, e.g., as container 12 and radioactive material 33, respectively.

Thus, e.g., and referring to U.S. Pat. No. 3,861,380 (the entire disclosure of which is hereby incorporated by reference into this specification), one may use as container 12 a “1. A radioactive-source projector which comprises: 1. a moveable casing including openings; 2. source-holder means in said casing and extendable through said openings, said source-holder means containing radioactive sources, and said source-holder means including a flexible tubular element that is closed at one end and adapted to be applied to the vicinity of a cancerous tissue to be treated in a living body, and that is opened at the other end for receiving said radioactive sources; 3. shield block means in said casing containing said source-holder means to afford protection against the radioactive sources positioned within said moveable casing; 4. flexible outer tube means receiving said one end of said source-holder means, said outer tube means having a small outer diameter and being adapted to be placed adjacent to the surface of a living body for treatment of cancerous tissue; 5. flexible ejection sheath means having one end connected to said shield block means and another end connected removably to said flexible outer tube for guiding said source-holder means from said shield block means to said flexible outer tube means; 6. actuating cable means removably coupled to said source-holder means for displacing said source-holder means through said flexible ejection sheath means; and 7. transfer means for transferring said actuating cable means and the associated source-holder means via said flexible ejection sheath means from said shield block means to said outer tube means and from said outer tube means to said shield block means.”

Thus, e.g., and referring to U.S. Pat. No. 3,872,856 (the entire disclosure of which is hereby incorporated by reference into this specification), one may use as container 12 “An apparatus for treating carcinoma of the walls and floor of the pelvic cavity comprising: an elongated hollow tube having a closed inner end adapted to be located in the pelvic cavity, the tube adapted to extend through a body opening to the outside of the body and including an opened outer end adapted to be located outside the body, means for locating radioactive material in the tube at the vicinity of said inner end by passing the radioactive material into the opened outer end of the tube and through the tube, positioning means including at least one inflatable balloon having a spacing portion attached to and surrounding the exterior of the tube in the vicinity of the said inner end thereof, said ballon, when inflated, spacing the walls and floor of the pelvic cavity from the radioactive material to position the radioactive material a generally uniform distance from all wall and floor surfaces subject to the radiation, while the tube extends through the body opening, and means for introducing fluid into the inflatable balloon spacing portion to expand the same and for removing fluid from the inflatable balloon spacing portion to collapse the same to permit the removal of the apparatus through the body opening.”

Thus, and referring to U.S. Pat. No. 4,323,055 (the entire disclosure of which is hereby incorporated by reference into this specification), one may use the radioactive seed described in such patent as radioactive material 33. There is claimed in such patent” “In a radioactive iodine seed comprising a sealed container having an elongate cavity, a therapeutic amount of radioactive iodine within said cavity and a carrier body disposed within said cavity for maintaining said radioactive iodine in a substantially uniform distribution along the length of said cavity, the improvement wherein said carrier body is an elongate rod-like member formed of silver or a silver-coated substrate which is X-ray detectable, said carrier body containing a layer of radioactive iodide formed on the surface of said carrier body, said carrier body occupying substantial portion of the space within said cavity.” One may use the carrier body of this patent as container 12, and the radioactive iodide as the radioactive material 33. The radioactive material 33 may be disposed inside the carrier body, and/or on it.

At column 1 of U.S. Pat. No. 4,323,055, it is disclosed that: “Radioactive iodine seeds are known and described by Lawrence in U.S. Pat. No. 3,351,049. The seeds described therein comprise a tiny sealed capsule having an elongate cavity containing the radioisotope adsorbed onto a carrier body. The seeds are inserted directly into the tissue to be irradiated. Because of the low energy X-rays emitted by iodine-125 and its short half-life, the seeds can be left in the tissue indefinitely without excessive damage to surrounding healthy tissue or excessive exposure to others in the patient's environment.” The iodine-125 may be used as the radioactive material 33.

U.S. Pat. No. 4,323,055 also discloses that: “In addition to the radioisotope and carrier body, the container also preferably contains an X-ray marker which permits the position and number of seeds in the tissue to be determined by standard X-ray photographic techniques. This information is necessary in order to compute the radiation dose distribution in the tissue being treated. The Lawrence patent illustrates two methods of providing the X-ray marker. In one embodiment, there is provided a small ball of a dense, high-atomic number material such as gold, which is positioned midway in the seed. The radioisotope is impregnated into two carrier bodies located on either side of the ball. In the other embodiment, the X-ray marker is a wire of a high-atomic number dense material such as gold located centrally at the axis of symmetry of a cylindrical carrier body. The carrier body is impregnated with the radioisotope and is preferably a material which minimally absorbs the radiation emitted by the radioisotope.” One may also utilize the X-ray marker of this patent in the assembly depicted in FIG. 1.

U.S. Pat. No. 4,323,055 also discloses that “In recent years iodine-125 seeds embodying the disclosure of the Lawrence patent have been marketed under the tradename “3M Brand I-125 Seeds” by Minnesota Mining and Manufacturing Company, the assignee of the present application. These seeds comprise a cylindrical titanium capsule containing two Dowex® resin balls impregnated with the radioisotope. Positioned between the two resin balls is a gold ball serving as the X-ray marker. These seeds suffer from several disadvantages. Firstly, the gold ball shows up as a circular dot on an X-ray film, and does not provide any information as to the orientation of the cylindrical capsule. This reduces the accuracy with which one can compute the radiation pattern around the capsule. Another disadvantage of using three balls inside the capsule is that they tend to shift, thereby affecting the consistency of the radiation pattern.” One may, e.g., use cylindrical titanium capsules as container 12.

At column 3 of U.S. Pat. No. 4,323,055, it is that disclosed radioactive iodine can be readily applied to the surface of a carrier body 3 by electroplating, stating that: “Silver is the material of choice for carrier body 3 because it provides good X-ray visualization and because radioactive iodine can be easily attached to the surface thereof by chemical or electroplating processes. It is obvious that other X-ray opaque metals such as gold, copper, iron, etc. can be plated with silver to form a carrier body . . . . Likewise, silver can be deposited (chemically or by using ‘sputtering’ and ‘ion plating’ techniques) onto a substrate other than metal, e.g., polypropylene filament . . . .” One may dispose the radioactive material 33 on the surface of the container 12 in addition to disposing it within the container 12 or instead of disposing it within the container 12.

By way of further illustration, and referring to U.S. Pat. No. 4,510,924 (the entire description of which is hereby incorporated by reference into this specification), one may use as container 12 “A radiation source for brachytherapy consisting essentially of: a sealed capsule having a cavity therein; and a brachytherapeutically effective quantity of americium-241 radioisotope disposed within said cavity, wherein the walls of said capsule consist essentially of a material having a thickness which (1) will transmit brachytherapeutically effective dosages of gamma radiation generated by said quantity of americium-241 and, (2) will contain the helium gas resulting from the decay of the alpha particles generated by said quantity of americium-241, and (3) which provides a neutron component of no more than approximately 1% of the total radiation dose provided by said source.” The radioactive material 33 may be, e.g., such americium-241.

U.S. Pat. No. 4,510,924 presents an excellent discussion of the state of the “radioactive material prior art” as of its effective filing date, Jun. 6, 1980. It discloses (at columns 1-3) that: “A wide variety of radioactive elements (radioisotopes) have been proposed for therapeutic use. Only a relatively small number have actually been accepted and employed on a large scale basis. This is due at least in part to a relatively large number of constraining considerations where medical treatment is involved. Important considerations are gamma ray energy, half-life, and availability.” The radioactive material discussed and referred to in such U.S. Pat. No. 4,510,924 may be used as radioactive material 33.

U.S. Pat. No. 4,510,924 also discloses that “An element employed almost immediately after its discovery in 1898, and one which is still in common use despite certain highly undesirable properties, is radium. By way of example, the following U.S. patents are cited for their disclosures of the use of radium in radiotherapy: Heublein U.S. Pat. No. 1,626,338; Clayton U.S. Pat. No. 2,959,166; and Rush U.S. Pat. No. 3,060,924.”

U.S. Pat. No. 4,510,924 also discloses that “A significant advantage in the use of radium for many purposes is its relatively long half-life, which is approximately 1600 years. The significance of a long half-life is that the quantity of radiation emitted by a particular sample remains essentially constant over a long period of time. Thus, a therapeutic source employing radium may be calibrated in terms of its dose rate, and will remain essentially constant for many years. Not only does this simplify dosage calculation, but long term cost is reduced because the source need not be periodically replaced.”

U.S. Pat. No. 4,510,924 also discloses that “However, a particularly undesirable property of radium is the requirement for careful attention to the protection of medical personnel, as well as healthy tissue of the patient. This is due to its complex and highly penetrating gamma ray emission, for example a component at 2440 keV. To minimize exposure to medical personnel, specialized and sometimes complicated “after loading” techniques have been developed whereby the radioisotope is guided, for example through a hollow tube, to the treatment region following the preliminary emplacement of the specialized appliances required.”

U.S. Pat. No. 4,510,924 also discloses that “In the past decade, cesium-137, despite a half-life of only 27 years, much shorter than that of radium, has gradually been displacing radium for the purpose of brachytherapy, especially intracavitary radiotherapy. Gamma radiation from cesium-137 is at a level of 660 keV compared to 2440 keV for the highest energy component of the many emitted by radium. This lower gamma energy has enabled radiation shielding to become more manageable, and is consistent with the recent introduction of the “as low as is reasonably achievable” (ALARA) philosophy for medical institutions. By way of example, the following U.S. patents are cited for their disclosures of the use of cesium-137 for radiotherapy: Simon U.S. Pat. No. 3,750,653; Chassagne et al U.S. Pat. No. 3,861,380; and Clayton U.S. Pat. No. 3,872,856. The Rush U.S. Pat. No. 3,060,924, referred to above for its disclosure of a radium source, also discloses the use of cesium-137.”

U.S. Pat. No. 4,510,924 also discloses that “Even more recently, the radioisotope iodine-125 has been employed for radiotherapy, particularly for permanent implants. A representative disclosure may be found in the Lawrence U.S. Pat. No. 3,351,049. Iodine-125, as well as other radioisotopes disclosed in the Lawrence U.S. Pat. No. 3,351,049, differ significantly from previously employed radioisotopes such as radium and cesium-137 in that the energy level of its gamma radiation is significantly lower. For example, iodine-125 emits gamma rays at a peak energy of 35 keV. Other radioisotopes disclosed in the Lawrence U.S. Pat. No. 3,351,049 are cesium-131 and palladium-103, which generate gamma radiation at 30 keV and 40 keV, respectively. Radioisotopes having similar properties are also disclosed in the Packer et al U.S. Pat. No. 3,438,365. Packer et al suggest the use of Xenon-133, which emits gamma rays at 81 keV, and Xenon-131, which generates gamma radiation at 164 keV.”

U.S. Pat. No. 4,510,924 also discloses that “Experience with such low energy gamma sources in radiotherapy has demonstrated that very low energy gamma rays, as low as 35 keV, can be highly effective for permanent implants. Significantly, such low gamma ray energy levels drastically simplify radiation shielding problems, reducing shielding problems to a level comparable to that of routine diagnostic radiology.”

By way of further illustration, one may use as container 12 the delivery system described in U.S. Pat. No. 4,697,575, the entire disclosure of which is hereby incorporated by reference into this specification. This patent claims: “A delivery system for interstitial radiation therapy comprising: an elongated member made from a material which is absorbable in living tissue, said member having a length substantially greater than its width, and a plurality of radioactive sources predeterminedly dispersed in said member, said elongated member having sufficient rigidity to be driven into a tumor without deflection to provide for controlled and precise placement of the radioactive sources in the tumor said elongated member comprising a plurality of separable segments, each segment having first and second complementary ends connectable to respective second and first ends of the adjacent segments”

As is disclosed in columns 3 and 4 of U.S. Pat. No. 4,697,575, “In the form shown in FIGS. 1-3, the non-deflecting member comprises a needle 20 formed by an elongated plastic body in which the seeds 22 are encapsulated axially aligned in spaced relationships. The needle has a tapered end 24 and a plurality of annular notches 26 are provided along the exterior surface in longitudinally spaced relation in the spaces between seeds so that the needle can be broken to provide the proper length dependent on the size of the tumor. In a typical case, the diameter of the needles is 1.06 mm. “The needles can be used in accordance with the following technique: 1. The tumor is exposed by a proper surgical technique. Alternatively, the tumor may be located by diagnostic methods using biplanar fluoroscopy, ultrasound or computerized tomography. 2. The size and shape of the tumor is determined. 3. The number of radioactive sources and spacing between the needles may be determined by the aforementioned nomograph technique developed by Drs. Kuam and Anderson. This calculation involves utilizing the average dimension and energy of the seeds as variables. 4. Each needle is inserted using one finger behind the tumor. When the end of the needle is felt bluntly, the proper depth has been reached. 5. Portions of the needles extending beyond the tumor are removed by breaking or cutting between or beyond the seeds. 6. After all the needles are in place, the surgical incision is closed, if the tumor has been exposed by surgical technique. 7. Dosimetry is monitored using stereo shift orthogonal radiographs and the appropriate computer program.”

By way of further illustration, and referring to U.S. Pat. No. 4,702,228 (the entire disclosure of which is hereby incorporated by reference into this specification), an implantable seed is disclosed and claimed. This patent claims: “A seed for implantation into a tumor within a living body to emit X-ray radiation thereto comprising at least one pellet that contains palladium enriched in palladium-102 to contain many times the amount naturally present, said palladium-102 being activatable by exposure to neutron flux so as to transform a portion of said palladium-102 to an amount of X-ray emitting palladium-103 sufficient to provide a radiation level measured as compensated mCi of greater than 0.5, and a shell of biocompatible material encapsulating said at least one pellet, said biocompatible material being selected from a material that is penetratable by X-rays in the 20-23 kev range.” Such palladium-102 may be used as the radioactive material 33.

At columns 1 et seq. of U.S. Pat. No. 4,702,228, it is disclosed that: “Advantages of interstitial implantation of radiation-emitting material for localized tumor treatment has been recognized for some time now. Interstitially implanted materials concentrate the radiation at the place where this treatment is needed, i.e., within a tumor so as to directly affect surrounding tumor tissue, while at the same time exposing normal tissue to far less radiation than does radiation that is beamed into the body from an external source.”

U.S. Pat. No. 4,702,228 also discloses that “One early implantable radioactive material was gold wire fragments enriched in radiation-emitting gold isotopes, such as gold-198. An advantage of gold wire, for interstitial implantation is that gold is compatible with the body in that it does not degrade or dissolve within the body. Another commonly used implantable material is radon-222. ” Each of these radioactive materials may be used as the material 33.

U.S. Pat. No. 4,702,228 also discloses that “Materials, such as gold-198 and radon-222, have significant counterindicating characteristics for interstitial tumor treatment in that they emit relatively penetrating radiation, such as X-rays or gamma radiation of higher energy than is preferred, beta particles or alpha particles. Such materials not only subject the patient's normal tissue to more destructive radiation than is desired but expose medical personnel and other persons coming into contact with the patient to significant doses of potentially harmful radiation.” Such gold-198 and radon-222 may be used as material 33.

U.S. Pat. No. 4,702,228 also discloses that “U.S. Pat. No. 3,351,049 describes capsules or seeds in which an enclosed outer shell encases an X-ray-emitting isotope having a selected radiation spectrum. Notably, the capsules contain iodine-125 having a radiation spectrum which is quite favorable for interstitial use compared to previously used materials. The encasing shell localizes the radioactive iodine to the tumor treatment site, preventing the migration of iodine to other parts of the body, notably the thyroid, which would occur if bare iodine were directly placed in the tumor site. The use of an encasing shell permits the use of other X-ray-emitting isotopes which would dissolve in the body or present a toxic hazard to the recipient . . . .” Such capsule with an X-ray emitting isotope disposed therein may be used as container 12.

U.S. Pat. No. 4,702,228 also discloses that “Other isotopes have been suggested as alternatives to iodine-125. The '049 patent, in addition to iodine-125, suggests palladium-103 and cesium-131 as alternatives. Palladium-103 has the advantage of being an almost pure X-ray emitter of about 20-23 keV. Furthermore, it is compatible with the body in that it is substantially insoluble in the body. Thus palladium presents less of a potential hazard to the body, in the rare event of shell leakage, than does radioactive iodine, which if it were to leak from its encasing shell, would migrate to and accumulate in the thyroid with potentially damaging results.” Such “other isotopes” also may be used as radioactive material 33.

U.S. Pat. No. 4,702,228 also discloses that “Although the '049 patent suggests the use of seeds containing palladium-103, to date, only seeds containing iodine-125 have been commercially available. The reason that palladium-103 has not been used as an interstitial X-ray source is suggested in Medical Physics Monograph No. 7, “Recent Advances in Brachytherapy Physics”, D. R. Shearer, ed., publication of the American Association of Physicists in Medicine, (1979) at page 19 where it is noted that its 17-day half-life (as compared with iodine-125 with about a 60-day half-life) is ‘just too short.’” Such palladium-103 may be used as the material 33.

U.S. Pat. No. 4,702,228 also discloses that “Indeed a 17-day half-life is difficult to work with in making capsules as produced according to the teachings of '049 patent in which substantially pure palladium-103 is contemplated. The short half-life represents a substantial obstacle to providing implants that contain substantially pure palladium-103. To produce substantially pure palladium-103, a transmutable element, such as rhodium-103, is converted to palladium-103 in a nuclear particle accelerator, and the palladium-103 is then isolated from untransmuted source material. The processing time of isolating the palladium-103 and additional processing time needed for encapsulating the radioactive material results in a substantial loss of activity of the palladium-103 before it is ever used in the body. Furthermore, producing palladium-103 by means of an atomic particle accelerator is difficult, and palladium-103 produced in this manner is very expensive. These considerations undoubtedly account for the fact that palladium-103 has not been incorporated in commercially available tumor treatment materials.”

U.S. Pat. No. 4,702,228 also discloses that “It is desirable to be able to use palladium-103 as an interstitially implantable X-ray source as the radiation spectrum of palladium-103 is somewhat more favorable relative to that of iodine-125. More importantly, the shorter half-life of palladium-103 relative to iodine-125, although presenting problems with respect to delivering the material to the patient, has important advantages with respect to patient care. The patient is significantly radioactive for a substantially shorter period of time and therefore poses less of a hazard to medical personnel and others who come in contact with the patient for the same period of time. By using a short half-life isotope for interstitial implantation, the time during which precautions against radiation exposure must be taken when treating the patient may be reduced, and the patient's periods of confinement in the hospital may be correspondingly reduced. As noted above, palladium does not present the potential problem of leaking iodine. Thus, it would be desirable to have methods and materials for making palladium-103 generally available as an implantable X-ray source.”

U.S. Pat. No. 4,702,228 also discloses that “A disadvantage of I-125-containing seeds, as presently produced, is that the seeds are anisotropic in their angular radiation distribution. This is due to the configuration of the capsules or seeds which are tubular and which, due to currently used shell-forming techniques, have large beads of encapsulating shell material at the sealed ends of the tubular structure. Although the '049 patent proposes unitary tubes that are sealed so as to have ends formed to be of substantially the same thickness as the sidewall of the tubular structure, the capsules actually produced by the assigness of the '049 patent have heavy beads of shell material at the ends of the seeds that result from the welding process. Such beads of material substantially shield emitted radiation, whereby the amount of radiation emitted from the ends of the capsule is substantially reduced relative to the amount of radiation emitted from the sidewall of the capsule.”

By way of further illustration, and referring to U.S. Pat. No. 4,784,116 (the entire disclosure of which is hereby incorporated by reference into this specification), one may use as container 12 the “container means” disclosed and claimed in such patent. U.S. Pat. No. 4,784,116 claims: A seed for implanting radiation-emitting material within a living body, comprising: radiation-emitting material; and a container means for sealingly enclosing said radiation-emitting material, including a tubular body of substantially uniform wall thickness having at least one open end and an end cap of wall thickness not substantially greater than that of said tubular body closing said open end, said end cap having an end wall and a generally tubular skirt portion depending from the periphery of said end wall and terminating in a free end, said skirt portion being at least partially received in the open end of said tubular body so as to engage said tubular body, said skirt portion and said tubular body interfitting and joined to each other to form a fluid-tight seal, so as to prevent contact between bodily fluids and said radiation-emitting material in said container.”

At column 2 of U.S. Pat. No. 4,784,116, it is disclosed that: “In order to function effectively, the radiation emitted from the radioisotope material must not be blocked or otherwise unduly attenuated. As indicated above, the small size of therapeutic seeds allows them to be inserted within the organ or tissue to be treated, so as to be totally surrounded thereby. Preferably, it is desirable that the radiation emitted from the radioisotope material have an equal distribution in all directions of emanation, i.e., have an isotropic radial distribution. In particular, it is generally desirable to avoid capsules with end constructions having a greater concentrations of radiation-absorbing material which obstructs the therapeutic radiation required for the successful treatment of affected tissues and organs.” The assembly 10 of FIG. 1 of this specification preferably has such an isotropic radial distribution of radiation from radioactive material 33.

By way of yet further illustration, one may use the as container 12 the capsule disclosed in U.S. Pat. No. 4,891,165, the entire disclosure of which is hereby incorporated by reference into this specification. This patent claims: “A small, metallic capsule for encapsulating radioactive materials for medical and industrial diagnostic, therapeutic and functional applications, comprising: at least first and second metallic sleeves, each of said sleeves comprising a bottom portion having a circumferential wall extending therefrom, and having an open and opposite said bottom portion; wherein said first sleeve has an outer surface which is complementary to and substantially the same size as the inner surface of said second sleeve, said second sleeve fitting snugly over the open end of said first sleeve, thereby forming a substantially sealed, closed capsule, having an inner cavity, with substantially uniform total wall thickness permitting substantially uniform radiation therethrough.”

The dimensions of the capsules of U.S. Pat. No. 4,891,165 are disclosed at columns 3-4 of the patent, wherein it is disclosed that: “In the embodiment shown in FIG. 1, it is desirable to construct a capsule having uniform dimensions so that radiation can pass therethrough in a relatively uniform pattern. The total thickness of sidewall 16 is substantially the same as the thickness of each bottom portion 13. When the two sleeves 11 and 12 are fitted together, a capsule is thus provided having walls of uniform total thickness. The thickness of the bottom portion 13 can vary with that of the wall portions 16, and further, the bottom portions of each sleeve can be varied so that any desired relationship between the total thickness of the walls and the bottom portions of the resulting capsule may be provided. The thickness of the bottom portions can range from about 0.05 mm to about 3.0 mm, while the thickness of the wall portions can range from about 0.03 mm to about 2.0 mm. The walls 16 of the sleeves are constructed so that the walls of the outer sleeve 12 are slightly longer than the walls of the inner sleeve 11 by approximately the thickness of the bottom portion 13 of the inner sleeve 11. For example, when the bottom portions of the sleeves have a thickness of 0.05 mm, the walls of the outer sleeve 12 will have a length which is 0.05 mm longer than the walls of the inner sleeve 11. This construction provides an ultimate capsule having uniform thickness when the sleeves 11 and 12 are interfitted. It will be appreciated that end portions 13 of the wall portions of each separate sleeve may be tapered toward the inner diameter of the sleeve so that insertion of the inner sleeve 11 into the outer sleeve 12 can be facilitated. The final outer dimensions of the capsules of the present invention have outer diameters which range from about 0.25 mm to about 25.0 mm and lengths which range from about 1.1 mm to about 25.0 mm. The sealed capsule includes a source of radiation, and may also contain a radiopaque marker material for viewing the location and orientation of the sealed capsule or seed in situ in a treatment site in a patient's body. Thus, capsules can be constructed of varying sizes, including minute capsules which, because of their thin walls, can contain an effective amount of a radioactive source. The complete internal structure of such seeds is described in applicant's copending application Ser. No. 07/225,302, filed Jul. 28, 1988, the entire disclosure of which is hereby incorporated by reference.” The container 12 of FIG. 1 may have similar dimensions, and it may also include a radiopaque marker.

By way of further illustration, one may use as container 12 the container means disclosed in U.S. Pat. No. 5,354,257, the entire disclosure of which is hereby incorporated by reference into this specification. This patent claims: “A minimally invasive intravascular medical device for providing a radiation treatment, comprising: a cylindrical first wire having a first uniform outer diameter and a longitudinally tapered distal end; a wire coil including a distal end, a proximal end, and a passageway extending longitudinally therebetween, said tapered distal end of said first wire extending longitudinally in said passageway of said wire coil, said proximal end of said wire coil being attached to said first wire, said coil having a second outer diameter within a predetermined tolerance of said first uniform outer diameter, said wire coil having a predetermined longitudinal curvature; a second wire having a distal end attached to said wire coil and a proximal end and extending longitudinally in said passageway to said tapered distal end of said first wire, said proximal end of said second wire being attached to said wire coil and said first wire in said longitudinal passageway; and a sleeve of radioactive material fixedly positioned at least partially around said second wire in said passageway a predetermined distance from said distal end of said wire coil.”

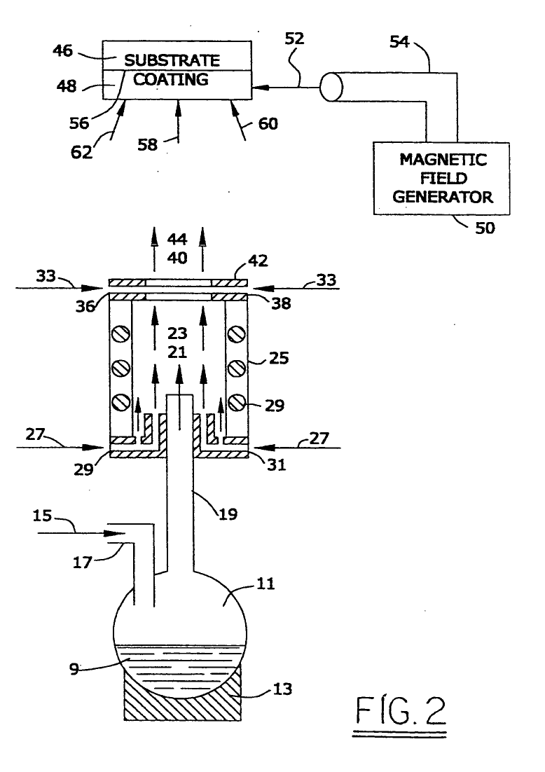

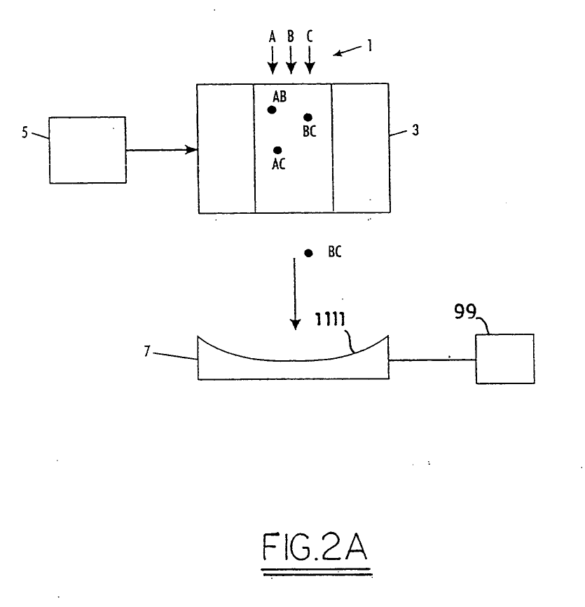

By way of yet further illustration, one may use as container 12 the seed disclosed in U.S. Pat. No. 5,405,309, the entire disclosure of which is hereby incorporated by reference into this specification. This patent claims “A seed for implantation into a tumor within a living body to emit X-ray radiation thereto comprising at least one pellet of an electroconductive support substantially non-absorbing of X-rays, having electroplated thereon a layer of a palladium composition consisting of carrier-free palladium 103 having added thereto palladium metal in an amount sufficient to promote said electroplating, said at least one electroplated pellet containing Pd-103 in an amount sufficient to provide a radiation level measured as apparent mCi of greater than 0.5, and a shell of a bicompatible material encapsulating said at least one electroplated pellet, said biocompatible material being penetrable by X-rays in the 20-23 kev range.” The shell preferably used in such device is described at column 7 of the patent, wherein it is disclosed that: “The shell 22 encapsulates the pellets 14 and the opaque marker 18 in such a way that the admixture of radioactive Pd-103/Pd cannot under normal circumstances come into contact with body tissue or fluids due to this encapsulating shell, thereby forming an additional barrier to escape and distribution of the radioactive isotope throughout the body. Accordingly, the outer shell is formed of a material that is biocompatible and preferably the encapsulating shell is titanium. The wall thickness of the titanium shell is about 0.001 to 0.005 inch, preferably 0.002 inch. Most advantageously, the shell will take the form of a tube with the ends thereof closed in a manner that precludes direct contact between body tissue and fluids and the internal components of the seed. This closure of the ends can be effected, for instance, by swaging shut the open ends and welding. Alternatively, the ends may be closed by capping them in a suitable manner, a preferred example of which is shown in FIG. 1 and FIG. 2. Referring to these figures, it is seen that the outer shell 22 is constructed from a three piece assembly, including the tube 24 and the pair of end caps 26 that are welded to the tube 24 after the other components, i.e., the X-ray-emitting pellets 14 and the X-ray-opaque marker 18 are inserted into the tube. The important advantage of this construction relative to the construction of the shells of seeds, some presently in commercial production, is that it permits the formation of thinner ends, i.e., about the same thickness as the sidewalls, and thereby provides for a better angular distribution of the emitted X-rays. Even though the shell material is selected to be as transparent to X-rays as is consistent with other requirements of the shell material, the shell will absorb some of the low-energy X-rays emitted by the palladium-103. By using end caps 26 having the same thickness as the tube 24, the end of the shell 22 is as thick as the sidewalls of the shell, promoting the generally isotropic angular distribution of X-rays from the seed. In the seed illustrated in FIG. 1, the end caps are cup-shaped, including a circular end wall 27 and an outwardly extending cylindrical sidewall 29. The diameter of the end caps 25 is proportioned to fit closely within the ends of the tube of the seed. After the seed 1 is assembled, the end caps 26 are welded, e.g., with a laser, to the tube 24, thereby permanently sealing the pellets 14 and the marker 18 within the shell. Although this construction produces double-walled sections extending outwardly of the circular end walls 27 of the end caps; a double-walled thickness is less than the thickness of end beads in some currently produced seeds, and the double-walled segment results in additional shielding only along a narrow angular region.”

The container 12 may be similar to the device depicted in U.S. Pat. No. 5,460,592, the entire disclosure of which is hereby incorporated by reference into this specification. This patent claims: “A carrier assembly containing radioactive seeds disposed within a bio-absorbable carrier material which is adapted to be inserted into a living tissue, said carrier assembly comprising: a seed carrier comprising an elongated member made of a carrier material absorbable in a living tissue and having a length substantially longer than its width; a plurality of predeterminedly spaced radioactive seeds disposed within said elongated member; a jig member having a plurality of first and second recesses therein, said first recesses having a shape to receive said seeds and said second recesses having a shape to receive said seed carrier; and, a removable sheath member disposed over said jig member, said sheath member having inner and outer surfaces, said inner sheath member surface being in slidable contact with at least a portion of said jig member; whereby, in use, said sheath member is disengageable from said jig member and at least a portion of said elongated member including at least one seed is removable from said jig member.”

At column 5 of U.S. Pat. No. 5,460,592, “I-125 Seeds” are described; these seeds may be used as radioactive material 33. It is disclosed that: “One seed presently available is Model No. 6711 available from Medi-Physics, Inc., an Amersham Company located in Arlington Heights, Ill., U.S.A. and referred to in Medi-Physics Bulletin No. TT0893A. The radioactive seeds are each welded titanium capsules containing I-125 absorbed onto a silver rod. The product, which is available from Amersham Holdings, Arlington Heights, Ill., is commercially known as I-125 Seeds®. Seeds 14 are spaced at predetermined dimensions in an elongated bio-absorbable material 15 whose length is substantially longer than its width. The carrier material is a flexible material and is absorbable in a living body. The material may be made of any of the natural or synthetic materials absorbable in a living body. Examples of natural absorbable materials as disclosed in U.S. Pat. No. 4,697,575 are the polyester amides from glycolic or lactic acids such as the polymers and copolymers of glycolate and lactate, polydioxanone and the like. Such polymeric materials are more fully described in U.S. Pat. Nos. 3,565,869, 3,636,956, 4,052,988 and European Patent Application 30822. Specific examples of absorbable polymeric materials that may be used to produce the substantially non-deflecting members of the present invention are polymers marketed by Ethicon, Inc., Somerville, N.J., under the trademarks “VICRYL” and “PDS”.”

By way of yet further illustration, one may use as container 12 the hollow-tube brachytherapy device disclosed in U.S. Pat. No. 5,713,828, the entire disclosure of which is hereby incorporated by reference into this specification. This patent claims: “A double-walled tubular brachytherapy device for interstitial implantation of radiation-emitting material within a living body, said double-walled tubular brachytherapy device comprising: an inner tubular element and an outer tubular element, said inner tubular element and said outer tubular element each having a first end and a second end, said inner tubular element and said outer tubular element being of a substantially equal length and said inner tubular element being substantially centrally disposed within said outer tubular element and spaced apart therefrom over substantially the entire length thereof, said first ends being sealingly joined and said second ends being sealingly joined; and wherein said inner tubular element comprises a tubular support having a lumen therethrough, an internal surface, and an external surface, said external surface having radiation-emitting material thereon.” At columns 1-4 of this patent, various “prior art seeds” are discussed. It is disclosed that: “In the prior art, brachytherapy “sources” are generally implanted for short periods of time and usually are sources of high radiation intensity. For example, irradiation of body cavities such as the uterus has been achieved by placing radium-226 capsules or cesium-137 capsules in the lumen of the organ. In another example, tumors have been treated by the surgical insertion of radium needles or iridium-192 ribbons into the body of the tumor. In yet other instances gold-198 or radon-222 have been used as radioactive sources. These isotopes require careful handling because they emit highly energetic and penetrating radiation that can cause significant exposure to medical personnel and to the normal tissues of the patient undergoing therapy. Therapy with sources of this type requires that hospitals build shielded rooms, provide medical personnel with appropriate protection and establish protocols to manage the radiation hazards.”

U.S. Pat. No. 5,713,828 also discloses that “The prior art interstitial brachytherapy treatment using needles or ribbons has features that inevitably irradiate normal tissues. For example, normal tissue surrounding the tumor is irradiated when a high energy isotope is used even though the radiation dose falls as the square of the distance from the source. Brachytherapy with devices that utilize radium-226, cesium-137 or iridium-192 is hazardous to both the patient and the medical personnel involved because of the high energy of the radioactive emissions. The implanted radioactive objects can only be left in place temporarily; thus the patient must undergo both an implantation and removal procedure. Medical personnel are thus twice exposed to a radiation hazard.”

U.S. Pat. No. 5,713,828 also discloses that “In prior art brachytherapy that uses long-term or permanent implantation, the radioactive device is usually referred to as a “seed.” Where the radiation seed is implanted directly into the diseased tissue, the form of therapy is referred to as interstitial brachytherapy. It may be distinguished from intracavitary therapy, where the radiation seed or source is arranged in a suitable applicator to irradiate the walls of a body cavity from the lumen.”

U.S. Pat. No. 5,713,828 also discloses that “Migration of the device away from the site of implantation is a problem sometimes encountered with presently available iodine-125 and palladium-103 permanently implanted brachytherapy devices because no means of affirmatively localizing the device may be available. The prior art discloses iodine seeds that can be temporarily or permanently implanted. The iodine seeds disclosed in the prior art consist of the radionuclide adsorbed onto a carrier that is enclosed within a welded metal tube. Seeds of this type are relatively small and usually a large number of them are implanted in the human body to achieve a therapeutic effect. Individual seeds of this kind described in the prior art also intrinsically produce an inhomogeneous radiation field due to the form of the construction.”

U.S. Pat. No. 5,713,828 also discloses that “The prior art also discloses sources constructed by enclosing iridium metal in plastic tubing. These sources are then temporarily implanted into accessible tissues for time periods of hours or days. These sources must be removed and, as a consequence, their application is limited to readily accessible body sites.” Such plastic tubing may be used as the containe 12, and such iridium metal may be used as radioactive material 33.

U.S. Pat. No. 5,713,828 also discloses that “Prior art seeds typically are formed in a manner that differs from isotope to isotope. The form of the prior art seeds is thus tailored to the particular characteristics of the isotope to be used. Therefore, a particular type of prior art seed provides radiation only in the narrow range of energies available from the particular isotope used.”

U.S. Pat. No. 5,713,828 also discloses that “Brachytherapy seed sources are disclosed in, for example, U.S. Pat. No. 5,405,165 to Carden, U.S. Pat. No. 5,354,257 to Roubin, U.S. Pat. No. 5,342,283 to Good, U.S. Pat. No. 4,891,165 to Suthanthirian, U.S. Pat. No. 4,702,228 to Russell et al, U.S. Pat. No. 4,323,055 to Kubiatowicz and U.S. Pat. No. 3,351,049 to Lawrence, the disclosures of which are incorporated herein by reference.” The containers 12, and radioactive materials 33 described in these patents may balso be used in the assembly 10 of this patent.

U.S. Pat. No. 5,713,828 also discloses that “The brachytherapy seed source disclosed by Carden comprises small cylinders or pellets on which palladium-103 compounded with non-radioactive palladium has been applied by electroplating. Addition of palladium to palladium-103 permits electroplating to be achieved and allows adjustment of the total activity of the resulting seed. The pellets are placed inside a titanium tube, both ends of which are sealed. The disclosed invention does not provide means to fix the seed source within the tissues of the patient to ensure that the radiation is correctly delivered. The design of the seed source is such that the source produces an asymmetrical radiation field due to the radioactive material being located only on the pellets. The patent also discloses the use of end caps to seal the tube and the presence of a radiographically detectable marker inside the tube between the pellets.”

U.S. Pat. No. 5,713,828 also discloses that “The patent to Roubin relates to radioactive iridium metal brachytherapy devices positioned at the end of minimally invasive intravascular medical devices for providing radiation treatment in a body cavity. Flexible elongated members are disclosed that can be inserted through catheters to reach sites where radiation treatment is desired to be applied that can be reached via vessels of the body.” One may use flexible, elongated members as container 12.

U.S. Pat. No. 5,713,828 also discloses that “The patent to Good discloses methods such as sputtering for applying radioactive metals to solid manufactured elements such as microspheres, wires and ribbons. The disclosed methods are also disclosed to apply protective layers and identification layers. Also disclosed are the resulting solid, multilayered, seamless elements that can be implanted individually or combined in intracavitary application devices.” The container 12 depicted in FIG. 1 may be made, in part, by conventional sputtering techniques.

U.S. Pat. No. 5,713,828 also discloses that “The patent to Suthanthirian relates to the production of brachytherapy seed sources and discloses a technique for use in the production of such sources. The patent discloses an encapsulation technique employing two or more interfitting sleeves with closed bottom portions. The open end portion of one sleeve is designed to accept the open end portion of a second slightly-smaller-diameter sleeve. The patent discloses the formation of a sealed source by sliding two sleeves together. Seeds formed by the Suthanthirian process may have a more uniform radiation field than the seed disclosed by Carden. However, the seed disclosed by Suthanthirian provides no means for securely locating the seed in the tissue of the patient.” The assembly 10 may be compried of “ . . . two or more interfitting sleeves with closed bottom portions (see, e.g., FIG. 1A of this specification).

U.S. Pat. No. 5,713,828 also discloses that “The patent to Russell et al. relates to the production of brachytherapy seed sources produced by the transmutation of isotopically enriched palladium-102 to palladium-103 by neutrons produced by a nuclear reactor. The Russell patent also discloses a titanium seed with sealed ends, similar to that of Carden, containing a multiplicity of components. A seed produced in this manner is associated with yielding a less than isotropic radiation field.”

U.S. Pat. No. 5,713,828 also discloses that “The patent to Kubiatowicz teaches a titanium seed with ends sealed by laser, electron beam or tungsten inert gas welding. The radioactive component of the seed is disclosed to be a silver bar onto which the radioisotope iodine-125 is chemisorbed. Seeds produced in this manner also tend to produce an asymmetric radiation field and provide no means of attachment to the site of application in the patient.” Such a “ . . . titanium seed with ends sealed by laser, electron beam, or tungsten inert gas welding . . .” may be used as the container 12.

U.S. Pat. No. 5,713,828 also discloses that “The patent to Lawrence discloses a radioactive seed with a titanium or plastic shell with sealed ends. Seeds are disclosed containing a variety of cylindrical or pellet components onto which one of the radioisotopes iodine-125, palladium-103 or cesium-131 is incorporated. The structure of the disclosed seeds yields a non-homogeneous radiation field and provides no means for accurately positioning the seed in the tissue that it is desired to irradiate.” One may use, e.g., a “. . . plastic shell with sealed ends . . .” as the container 12.

By way of yet further illustration, one may use the brachytherapy source disclosed in U.S. Pat. No. 5,997,463, the entire disclosure of which is hereby incorporated by reference into this specification. This United States patent describves a needle guide for a prostate implant stabiliziation device. As is disclosed in column 1 of this patent, “Brachytherapy has been successfully used in the treatment of prostate cancer particularly with the development of a number of implant stabilization devices used in conjunction with ultrasound probes so that the prostate gland can be viewed and seeds implanted by patterns of needles held by specially designed needle holding devices while viewing the inflicted area. Obviously, it is necessary to have full freedom of movement of the ultrasound probe as well as the needle holder to identify the inflicted area and position the instrumentalities to seed the area effectively. There are a number of prostate implant stabilization devices on the market such as the Northwest Transperineal device marketed by Seed Plan Pro in Seattle, Wash. and the Universal Stepping and Stabilizing System for seed implementation marketed by Devmed, Inc. located in Singer Island, Fla. In addition, Tayman Medical, Inc. located in St. Louis, Mo. markets a stepping and stabilization system under the trademark ACCUSEED. All of the units presently marketed utilize metallic and permanent needle guides which, after use, must be meticulously cleaned in every needle opening with specially designed brushes so that no bacteria or other foreign substances are present after the cleaning takes place. Moreover, these needle guides are self sustaining and self supporting except to the extent they have supporting members that may be adjustable received within other components of the stabilizing system.” Such needle guides may be used as the container 12.

The needle guide claimed in U.S. Pat. No. 5,957,935 is: “A needle guide and holding bracket for a prostate implant stabilization device comprising: a base; a movable platform carried by the base, the platform having a horizontally adjustable needle guide support; a needle guide holding bracket vertically adjustable with respect to the needle guide support, the needle guide holding bracket including an inverted U-shaped body having a needle guide receiving opening and two depending legs cooperating with the needle guide support to allow vertical movement and fixed positioning of the holding bracket; and a disposable needle guide cooperatively received and carried by the holding bracket.”

A discussion of “prior art” brachytherapy sources is presented at columns 1-3 of U.S. Pat. No. 5,997,463, wherein it is disclosed that: “Over the years, brachytherapy sources implanted into the human body have become a very effective tool in radiation therapy for treating diseased tissues, especially cancerous tissues. The brachytherapy sources are also known as radioactive seeds in the industry. Typically, these brachytherapy sources are inserted directly into the tissues to be irradiated using surgical methods or minimally invasive techniques such as hypodermic needles. These brachytherapy sources generally contain a radioactive material such as iodine-125 which emits low energy X-rays to irradiate and destroy malignant tissues without causing excessive damage to the surrounding healthy tissue, as disclosed by Lawrence in U.S. Pat. No. 3,351,049 ('049 patent). Because radioactive materials like iodine-125 have a short half-life and emit low energy X-rays, the brachytherapy sources can be left in human tissue indefinitely without the need for surgical removal. However, although brachytherapy sources do not have to be removed from the embedded tissues, it is necessary to permanently seal the brachytherapy sources so that the radioactive materials cannot escape into the body. In addition, the brachytherapy source must be designed to permit easy determination of the position and the number of brachytherapy sources implanted in a patient's tissue to effectively treat the patient. This information is also useful in computing the radiation dosage distribution in the tissue being treated so that effective treatment can be administered and to avoid cold spots (areas where there is reduced radiation).

U.S. Pat. No. 5,997,463 also discloses that “Many different types of brachytherapy sources have been used to treat cancer and various types of tumors in human or animal bodies. Traditional brachytherapy sources are contained in small metal capsules, made of titanium or stainless steel, are welded or use adhesives, to seal in the radioactive material.”

U.S. Pat. No. 5,997,463 also discloses that “These various methods of permanently sealing the brachytherapy sources, used so that the radioactive materials cannot escape into the body and do not have to be removed after treatment, can have a dramatic effect on the manufacturing costs and on the radiation distribution of the brachytherapy sources. Increased costs reduce the economic effectiveness of a brachytherapy source treatment over more conventional procedures such as surgery or radiation beam therapy. In addition, the poorer radiation distribution effects, due to these sealing methods, in conventional brachytherapy sources may ultimately affect the health of the patient, since higher doses of radiation are required or additional brachytherapy sources must be placed inside the human body. All which leads to a less effective treatment that can damage more healthy tissue than would otherwise be necessary.”

U.S. Pat. No. 5,997,463 also discloses that “A first type of conventional brachytherapy source 10 is shown in FIG. 1, and uses two metal sleeves 12 and 14. The brachytherapy source 10 is disclosed in U.S. Pat. No. 4,891,165 issued Jun. 2, 1990 to Sutheranthiran and assigned to Best Industries of Springfield Va. Each of the sleeves has one closed end 16 and 18 using die-drawn techniques. Sleeve 14 has an outer diameter that is smaller than an inner diameter of the sleeve 12 to permit the sleeve 14 to slide inside sleeve 12 until the open end of sleeve 14 contacts the closed end 16 of the sleeve 12. Radioactive material, such as pellets, are placed inside the smaller sleeve 14, and then the larger external sleeve 12 is slid over the smaller sleeve 14. Next, the brachytherapy source 10 is permanently sealed by TIG (Tungsten Inert Gas) welding the open end of the larger sleeve 12 to the closed end 18 of the smaller sleeve 14. Laser welding may also be used. Although the welding of the two sleeves 12 and 14 together provides a good seal, the brachytherapy source 10 suffers from several drawbacks.” The sleeve 10 of U.S. Pat. No. 5,997,463 may be used as the container 12 of the instant case.

U.S. Pat. No. 5,997,463 also discloses that “One drawback results from the radiation seed 10 being formed from two distinctly different sized pieces (the two sleeves 12 and 14), which involves an additional assembly step of fitting the two sleeves 12 and 14 together. This is time consuming and can slow the assembly process down, as well as increase the overall cost of producing the brachytherapy sources 10.”

U.S. Pat. No. 5,997,463 also discloses that “Another conventional brachytherapy source 30, as shown in FIG. 2, uses a single tube 32 which has end caps 34 and 36 inserted at the ends 38 and 40 of the single tube 32 to hold the radioactive material. The brachytherapy source 30 is disclosed in U.S. Pat. No. 4,784,116 issued Nov. 15, 1988 to Russell, Jr. et al. and assigned to Theragenics Corporation of Atlanta, Ga. The ends 38 and 40 are then welded, or adhesively secured, to the end caps 34 and 36 to close off and seal the brachytherapy source 30. Although the brachytherapy source 10 provides a single wall and a better radiation distribution along the length (or sides) of the brachytherapy source 30, the brachytherapy source 30 still suffers from several drawbacks.”

U.S. Pat. No. 5,997,463 also discloses that “A first drawback is that the ends 38 and 40 of the brachytherapy source 30 do not provide a uniform radiation distribution approximating a point source, because the end caps 34 and 36 provide a double wall at the end of the brachytherapy source 30 that blocks off a substantial amount of radiation. A further drawback results form the welds used to seal the end caps 34 and 36 to the ends 38 and 40 of the singe tube 32, since these also reduce the radiation distribution. Another drawback results from there being a three-step assembly process; rather, than the two step assembly process discussed above, since there are now three separate parts to be assembled together (the single tube 32 and the end caps 34 and 36).”

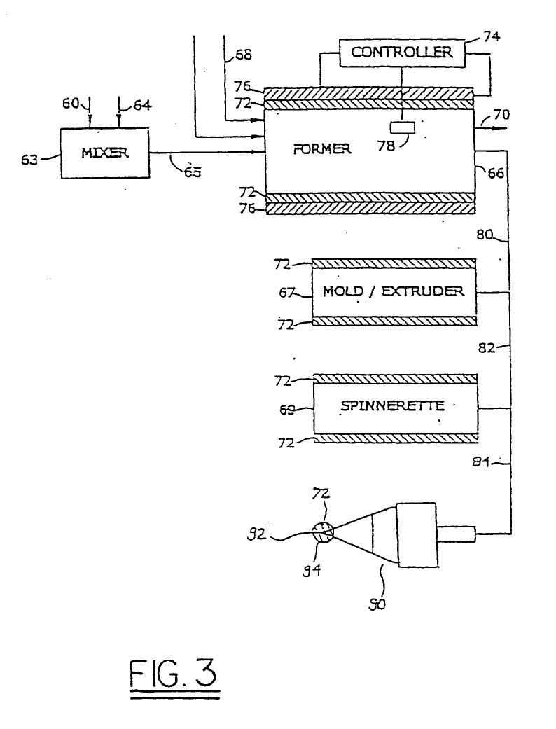

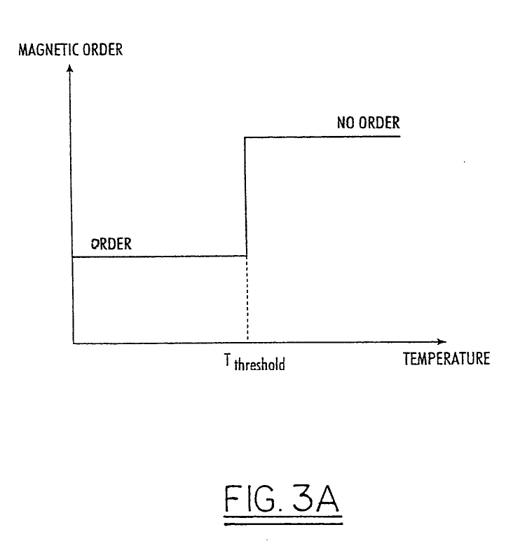

U.S. Pat. No. 5,997,463 also discloses that “In an alternative to this type of conventional brachytherapy source, a brachytherapy source 50, as shown in FIG. 3, has end plugs 52 and 54 that are slid into the open ends of a single tube 56. The brachytherapy source 50 is disclosed in U.S. Pat. No. 5,683,345 issued Nov. 4, 1997 to Waksman et al. and assigned to Novoste Corporation of Norcross, Ga. The end plugs 52 and 54 are either secured in place with an adhesive and the metal of the single tube 56 is then bent around the end plugs 52 and 54, or the end plugs 52 and 54 are welded to the single tube 56. The brachytherapy source 50 suffers from the same drawbacks as discussed above. In addition, the radiation distribution out the end plugs 52 and 54 is substantially reduced due to the added thickness of the end plugs 52 and 54.”

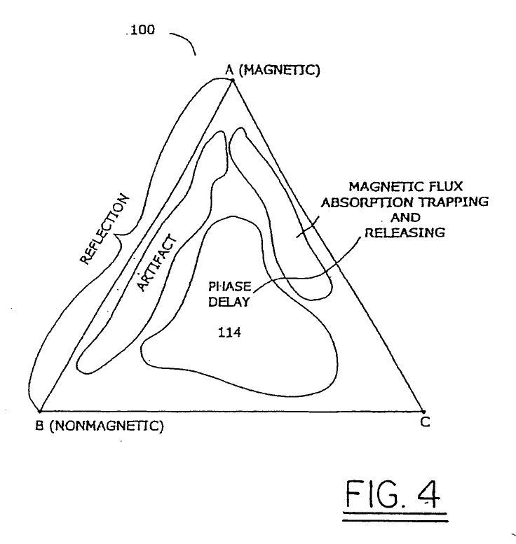

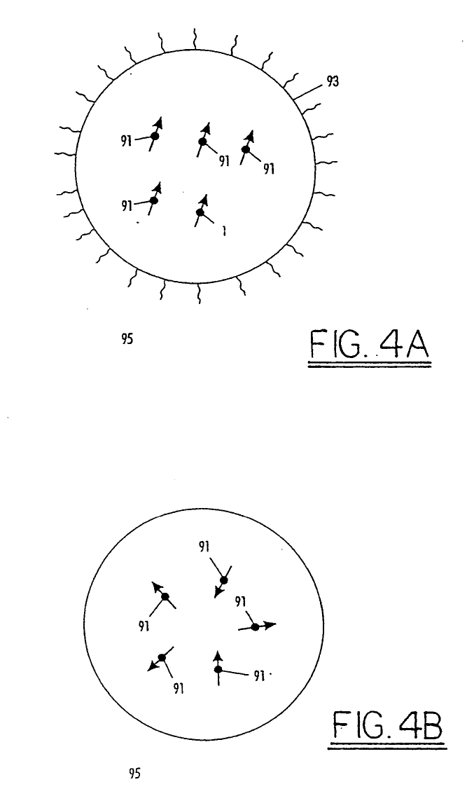

U.S. Pat. No. 5,997,463 also discloses that “In another conventional brachytherapy source 70, as shown in FIG. 4, some of the drawbacks of the multiple piece assembly are overcome by using a single tube 72 to provide a body with a uniform side wall along the length of the brachytherapy source 70. The brachytherapy source 70 is distributed by Amersham International PLC. One end 74 of the single tube 72 is TIG welded, and then the radioactive material is inserted into the open end 76 of the single tube 72. Next the open end 76 is TIG welded to seal the single tube 72 to provide a single unitary brachytherapy source structure. However, the brachytherapy source 70 suffers from many drawbacks.”

U.S. Pat. No. 5,997,463 also discloses that “For example, TIG welding the ends 74 and 76 causes formation of a bead of molten metal at the ends 74 and 76 of the single tube 72. Due to the nature of TIG welding the welded ends 74 and 76 generally form a bead that may be as thick as the diameter of the single tube 72. Therefore, the radiation distribution is substantially diminished out of the ends 74 and 76 of the brachytherapy source 72 due to the thickness of the beads 78 and 80 closing off the ends 74 and 76. In addition, the end 76 is only closed after the radioactive material is inserted into the single tube 72, and the end 76 may not seal in the same manner due to the presence of the radioactive material carrier body effecting the thermal characteristics of the brachytherapy source 70. Thus, the bead 80 can be a different shape than the bead 78, which may further alter the radiation distribution and could lead to inconsistent radiation distributions from one brachytherapy source to another, making the prediction of the actual radiation distribution more difficult.”

U.S. Pat. No. 5,997,463 also discloses that “Therefore, although the brachytherapy source 70 overcome some of the drawbacks in the earlier brachytherapy sources by minimizing the assembly steps associated with multiple pieces, it does not provide an even radiation distribution. In fact, due to the potential for variations of the second end during the TIG welding, the distribution can vary substantially from brachytherapy source 70 to brachytherapy source 70. Typical radiation distribution patterns for conventional brachytherapy sources 70 using the single tube 72 are shown in FIGS. 5(a) and 5(b). As is shown in FIGS. 5(a) and 5(b), the radiation distribution patterns 102 and 104 tend to diminish substantially toward the ends 74 and 76 of the brachytherapy source 70 and form cold zones 106 and radiation lobes 108. This means that depending on how the brachytherapy sources 70 are placed adjoining each other, there may be cold spots in the radiation distribution between adjoining brachytherapy sources 70, where cells are not receiving radiation from the cold zones 106 at the ends 74 and 76. Or if the adjoining brachytherapy sources are placed close enough together, to assure no cold spots from the presence of the cold zones 106, there will be overlapping areas in the radiation lobes 108 that may provide an excessive dose of radiation. Either of these two conditions could result in either too much or too little radiation, which results in a less effective medical treatment.”

By way of yet further illustration, one may use the process disclosed in U.S. Pat. No. 6,086,942 for preparing a brachytherapy source. This patent claims: “A method for making a radiation-emitting element, comprising the steps of: depositing a radioactive fluid from a fluid-jet printhead onto a surface of a brachytherapy device, said radioactive fluid comprising a radioactive isotope in a radiation-resistant curable liquid, said curable liquid comprising a carrier solvent; wherein said fluid is deposited in a predetermined pattern.”

As is disclosed at columns 8 et seq. of U.S. Pat. No. 6,086,942, “In accordance with the present method, a brachytherapy support element is positioned at successive predetermined positions in front of the printhead of a fluid-jet printer so that the fluid is applied in a predetermined pattern. In a preferred embodiment . . . measurement of the amount of radioactive material deposited on the brachytherapy seed is done during the manufacturing process, and the information derived is used to adjust the printing parameters so as to keep the product to a desired specification . . . . ”

U.S. Pat. No. 6,086,942 also discloses that “The method of the present invention may also comprise applying a substantially radiation-transparent sealing layer over the radioactive-material-coated brachytherapy support element, so as to sealingly enclose the radiation-emitting material. In different embodiments of a device made by the method of the present invention, the sealing layer may be a plastic coat, a titanium shell, or other suitable radiation-transparent material.”

U.S. Pat. No. 6,086,942 also discloses that “FIG. 2 is a flow chart that illustrates the flow of parts in an assembly process and the flow of data to a computing means which commands a printhead to print radioactive fluid onto the inner tube of a seed of the type disclosed in the '828 patent. Also shown is the flow of parts and data associated with the assembly of the inner tube and a sealing layer into a finished brachytherapy device. In FIG. 2, data flow is indicated with dashed arrows and material flow is indicated with solid arrows. FIG. 2 shows a diagrammatic representation of the stations of a brachytherapy seed production line. An inner tube is loaded onto a conveyor at loading station 021, and the X-ray absorption by the inner-tube wall is measured at measuring station 022. An outer tube is loaded onto a conveyor at loading station 023, and the X-ray absorption by the outer-tube wall is measured at measuring station 024. The outer tube is then passed to assembly station 028. Radioactive fluid is printed on the surface of the inner tube at printing station 025, the fluid is cured at curing station 026, the activity of the printed tube is measured at radiation, measuring station 027 and the printed, cured inner tube is passed to assembly station 028. At assembly station 028 the outer tube is placed over the printed inner tube and the assembly is passed to sealing station 029 where the inner tube is sealingly attached to the outer tube. Quality control is achieved by measuring the properties of finished seeds. Computer 030 receives data from measuring stations 022, 024 and 027 and controls the amount and position of deposition of radioactive fluid at printing station 025. Measuring station 027 comprises two opposed radiation detectors equally spaced from a seed from which the radiation is to be measured. In an embodiment of the present invention wherein Pd-103 is the isotope, cadmium zinc telluride (CZT) detectors are used.”

U.S. Pat. No. 6,086,942 also discloses that “An apparatus similar to a jeweler's lathe was used to carry out a process of the present invention. The apparatus included the features schematically shown in FIG. 3. As depicted, variable speed motor 101 is mounted to drive driven-spindle 102. Titanium tube 103 is mounted. between driven-spindle 102 and free-spindle 104. Printhead 105 is mounted so that printhead nozzle plate 106 is at least 0.1 and not more than 3 mm from the surface of titanium tube 103. Pulsed LED light source 107 is mounted adjacent to gap 109 between printhead-face 106 and titanium tube 103. Monitoring video-camera 108 is mounted to observe drops (not shown) illuminated by LED light source 107 as they fly between printhead nozzle plate 106 and titanium tube 103 across gap 109. LED light source 107 also illuminates the build-up of fluid (not shown) on surface of titanium tube 103. Tube 110 directs a gentle, hot, dry stream of gas onto the printed surface of titanium tube 103 to speed the drying or curing of the printed drops.”

By way of yet further illustration one may use as container 12 the brachy seeds disclosed and claimed in U.S. Pat. No. 6,099,458, the entire disclosure of which is hereby incorporated by reference into this specification. This patent claims: “An essentially cylindrical, metal-encapsulated, brachytherapy source comprising: an outer metal capsule, an annulus in a central interior position of said outer metal capsule, and a longitudinally extending heavy metal core in said annulus; said annulus being made of the same metal as said outer metal capsule; means including one or more low-profile welds around the central circumference of said outer metal capsule for attaching said outer metal capsule to said annulus and for sealing said outer metal capsule; a plurality of substrate particles each having bound thereto a radioisotope, said substrate particles being positioned in said outer metal capsule so that the radioisotope is distributed symmetrically within the source, equally divided between the two ends of the source, and positioned with a strong bias towards the extremes of the two ends of the source; and the length of said metal core being determined by the shape, size and number of substrate particles at each end of the source.”

In column 6 of U.S. Pat. No. 6,099,458, the preparation of zeolite beads bound to palladium is disclosed. It is stated that: “It is intended to produce one hundred titanium-encapsulated interstitial brachytherapy sources each containing six millicuries of palladium-103 radioactivity. The palladium-103 in each source is to be divided between four zeolite bead substrates distributed as follows: two millicuries on each outer bead and one millicurie on each inner bead. The sources are to have dimensions as follows: length 4.5 millimeters; diameter 0.8 millimeters, and end-tube wall thickness 0.05 millimeters.” These zeolite beads bound to palladium may be used as radioactive material 33.

U.S. Pat. No. 6,086,942 also discloses that “A large bath of 4A type zeolite beads having bead diameters of 0.65 millimeters is previously acquired. Large batches of each of the capsule parts are acquired in the following dimensions: end-tube, 2.2 millimeters in length, 0.8 millimeters in outer diameter, 0.05 millimeters in wall thickness; and titanium/platinum-iridium alloy annular plugs, 1.7 millimeters in length, 0.7 millimeters in body diameter, core diameter 0.3 millimeters, ridge diameter 0.75 millimeters, and ridge width 0.1 millimeters. The annular plugs are sized to fit snugly into the end tubes so that when press fitted the two pieces do not easily part.”

U.S. Pat. No. 6,086,942 also discloses that “A sub-batch of at least two hundred of the 4A zeolite beads is suitably immersed in and mixed with an aqueous solution of palladium-103 in ammonium hydroxide at a pH of 10.5 so as to evenly load 2 millicuries of palladium-103 onto each bead. The beads are then separated from the solution and thoroughly dried in a drying oven, first at 100 degrees Celsius for 1 hour and then at 350 degrees Celsius for 1 hour. Another sub-batch of at least two hundred of the zeolite beads is taken and similarly treated so as to yield dry zeolite beads each loaded with 1 millicurie of palladium-103.”

U.S. Pat. No. 6,086,942 also discloses that “A zeolite bead loaded with 2 millicuries of palladium-103 is dispensed into each of two hundred titanium end-tubes held in a vertical orientation with the open ends uppermost. Then a zeolite bead loaded with 1 millicurie of palladium-103 is dispensed into each of the same two hundred end-tubes, so that a 1 millicurie bead rests on top of each 2 millicurie bead. A titanium annular plug with a platinum-iridium alloy core is then pressed firmly into each of the open ends of one hundred of the end-tubes into which the zeolite beads have been dispensed. The pressure used is just sufficient to ensure that the perimeter of the previously open end of the end-tube rests squarely against the ridge stop on the annular plug. The one-hundred plugged end-tubes are then inverted and each is pressed, protruding annular plug first, into one of the remaining one hundred unplugged end-tubes. Each of the one hundred assembled sources is then laser welded under argon atmosphere to provide a hermetic seal around the circumference where the previously open ends of the two end-tubes and the ridge of the annular plug meet. The sources are then ready for surface cleaning, inspection and testing before shipment to medical centers.”

By way of yet further illustration, one may use the brachy seed assemblies disclosed in U.S. Pat. No. 6,132,359, the entire disclosure of which is hereby incorporated by reference into this specification. This patent discusses the “isotopic radial distribution” of the ideal brachy seed; the seed assembly 10 of FIG. 1 preferably has, in one embodiment, this “isotopic radial distribution.” At columns 1-2 of this patent, it is disclosed that: “In order to function effectively, the radiation emitted from the radioisotope within the seed cannot be blocked or otherwise unduly attenuated. Preferably, radiation emitted from the radioisotope is uniformly distributed from the seed in all directions, i.e., has an isotropic radial distribution. In particular, it is generally desirable to avoid seeds having end constructions having a greater concentration of radiation-absorbing material, which attenuates the therapeutic radiation required for the successful treatment of diseased tissue.”

U.S. Pat. No. 6,132,359 also discloses that “Providing a uniform distribution of radiation from a seed has been difficult to impossible to accomplish. For example, present-day seeds have a radioisotope adsorbed onto a carrier substrate, which is placed into a metal casing that is welded at the ends. The most advantageous materials of construction for the casing which encapsulates the radioisotope-laden carrier are stainless steel, titanium, and other low atomic number metals. However, problems exist with respect to sealing casings made from these materials. Such metallic casings typically are sealed by welding, but welding of such small casings is difficult because welding can locally increase the casing wall thickness, or can introduce higher atomic number materials at the ends of the casing where the welds are located. The presence of such localized anomalies can significantly alter the geometrical configuration at the welded ends, resulting in undesirable shadow effects in the radiation pattern emanating from the seed. Such seeds also have the disadvantage of providing a nonhomogeneous radiation dose to the target due to their construction, i.e., the relatively thick ends attenuate the radiation more than the relatively thin body of the seed.”

U.S. Pat. No. 6,132,359 also discloses that “Other methods of forming the seed casing include drilling a metallic block to form a casing, and plugging the casing to form a seal. However, this method suffers from the disadvantage that a casing of uniform wall thickness is difficult to obtain, and the radiation source, therefore, is not able to uniformly distribute radiation.” One or more of these methods may be used to form the container 12.

The object of U.S. Pat. No. 6,132,359 was to provide brachytherapy seeds with a relatively uniform radiation dose. The patent claims: “An elongated brachytherapy seed comprising a radioisotope-laden carrier disposed within a sealed casing, wherein (a) the casing has a center portion of a first diameter and end portions each having a diameter that is substantially smaller than the first diameter, and (b) the radioisotope-laden carrier is acicular and has a polygonal cross section, wherein the carrier has one end of the carrier rotated around the longitudinal axis of the carrier.”

By way of yet further illustration, one may use the process of U.S. Pat. No. 6,163,947 to make a hollow-tube brachytherapy device; the entire disclosure of this United States patent is hereby incorporated by reference into this specification. This patent claims: “A method of making a sealed double-walled tubular brachytherapy device having a lumen therethrough for interstitial implantation of radiation-emitting material within a living body, said method comprising: fabricating an inner tubular element, said inner tubular element being fabricated to have an external surface, a lumenal surface, a first open end, a second open end, and a lumen continuous with said first open end and said second open end; fabricating an outer tubular element, said outer tubular element being fabricated to have a first open end, a second open end, and a lumen continuous with said first open end and said second open end, said tubular element also being fabricated to be of substantially equal length to said tubular support and of a diameter sufficient to permit said tubular support to be positioned within said lumen of said tubular element; depositing a layer of radiation-emitting material on said external surface of said inner tubular element; positioning said inner tubular element within said outer tubular element so that said inner tubular element is disposed coaxially and substantially centrally within said outer tubular element and spaced apart therefrom; sealingly joining said first open end of said inner tubular element and said first open end of said outer tubular element; and sealingly joining said second open end of said inner tubular element and said second open end of said outer tubular element, so as to form said sealed double-walled tubular brachytherapy device.”

By way of yet further illustration, one may use the seed delivery system disclosed in U.S. Pat. No. 6,221,003, the entire disclosure of which is hereby incorporated by reference into this specification. This patent claims: U.S. Pat. No. 6,221,003 claims: “A brachytherapy seed delivery system comprising: a seed cartridge including a central channel; a seed cover removably attached to said channel; a plurality of brachytherapy seeds disposed within said central channel; and a plurality of absorbable, dimensionally stable spacers disposed within said central channel, wherein said absorbable, dimensionally stable spacers are interspersed between said brachytherapy seeds.”

U.S. Pat. No. 6,221,002 discloses a seed delivery system for prostate cancer. As is disclosed at columns 1-2 of this patent, “Prostate brachytherapy can be divided into two categories, based upon the radiation level used. The first category is temporary implantation, which uses high activity sources, and the second category is permanent implantation, which uses lower activity sources. These two techniques are described in Porter, A. T. and Forman, J. D., Prostate Brachytherapy, CANCER 71: 953-958, 1993. The predominant radioactive sources used in prostate brachytherapy include iodine-125, palladium-103, gold-198, ytterbium-169, and iridium-192. Prostate brachytherapy can also be categorized based upon the method by which the radioactive material is introduced into the prostate. For example, a open or closed procedure can be performed via a suprapubic or a perineal retropubic approach.”

U.S. Pat. No. 6,221,003 also discloses that “Prostate cancer is a common cancer for men. While there are various therapies to treat this condition, one of the more successful approaches is to expose the prostate gland to radiation by implanting radioactive seeds. The seeds are implanted in rows and are carefully spaced to match the specific geometry of the patient's prostate gland and to assure adequate radiation dosages to the tissue. Current techniques to implant these seeds include loading them one at a time into the cannula of a needle-like insertion device, which may be referred to as a brachytherapy needle. Between each seed may be placed a spacer, which may be made of catgut. In this procedure, a separate brachytherapy needle is loaded for each row of seeds to be implanted. Typically, if a material such as catgut is used as a spacing material the autoclaving process may make the spacer soft and it may not retain its physical characteristics when exposed to autoclaving. It may become soft, change dimensions and becomes difficult to work with, potentially compromising accurate placement of the seeds. Alternatively, the seeds may be loaded into the center of a suture material such as a Coated VICRYL (Polyglactin 910) suture with its core removed. In this procedure, brachytherapy seeds are carefully placed into the empty suture core and loaded into a needle-like delivery device. Although Coated VICRYL suture is able to withstand autoclaving, the nature of its braided construction can make the exact spacing between material less than desirable.”

U.S. Pat. No. 6,221,003 also discloses that “It would, therefore, be advantageous to design a seed delivery system utilizing a plurality of spacers which are absorbable and which do not degrade significantly when subjected to typical autoclave conditions. It would further be advantageous to design a method of loading a brachytherapy seed delivery system utilizing a plurality of spacers which are absorbable and which do not degrade significantly when subjected to typical autoclave conditions. It would further be advantageous to design an improved brachytherapy method utilizing a plurality of spacers which are absorbable and which do not degrade significantly when subjected to typical autoclave conditions.”

Referring again to FIG. 1, and in the preferred embodiment depicted therein, the sealed container 12 may be any of the prior art brachy seed containers described elsewhere in this specification. Alternatively, or additionally, one may use or more of the containers for radioactive material disclosed, e.g., in U.S. Pat. Nos. 2,269,458, 2,959,166, 3,750,653, 4,784,116, 4,891,165, 5,405,309, and the like. The entire disclosure of each of these United States patents is hereby incorporated by reference in to this specification.

U.S. Pat. No. 2,269,458 discloses: “A capsule for containing a radioactive substance and constructed of a metal capable of being attracted by a magnet.” This capsule comprises “ . . . a substantially conical tip portion 10 of duralumin or other lightweight metal permeable to the gamma ray emanations of a radium pellet 11 contained in a socket formed in an axially disposed screw threaded nipple 12. The socket . . . is formed of a ferrous metal capable of being attracted and supported by the pole piece of a magnet 14.” Such a capsule may be used as the container 10 of this invention.

U.S. Pat. No. 3,370,653 claims: “A capsule adapted to be inserted in and retained by the uterus, comprising an elongated and enlarged bulbous body portion with a cavity therein, said cavity being disposed generally longitudinally within said body portion and having a diameter sufficient to accommodate a source of radioactive material therein, a thin-walled narrow tube connected to said body portion and arranged coaxially with said cavity so as to permit insertion of a radioactive source into said cavity through said tube, the outside diameter of said tube being not greater than 2 mm. so as to permit said capsule to be retained within and tolerated by the uterus with said tube projecting through the cervical so that said source may be inserted into the cavity after the capsule is positioned in the uterus.” Such a capsule may be used as the container 10 of this invention.

U.S. Pat. No. 4,784,116 describes and claims: “A seed for implanting radiation-emitting material within a living body, comprising: radiation-emitting material; and a container means for sealingly enclosing said radiation-emitting material, including a tubular body of substantially uniform wall thickness having at least one open end and an end cap of wall thickness not substantially greater than that of said tubular body closing said open end, said end cap having an end wall and a generally tubular skirt portion depending from the periphery of said end wall and terminating in a free end, said skirt portion being at least partially received in the open end of said tubular body so as to engage said tubular body, said skirt portion and said tubular body interfitting and joined to each other to form a fluid-tight seal, so as to prevent contact between bodily fluids and said radiation-emitting material in said container.” Such “container means” may be used as the container 12 of t his invention.

U.S. Pat. No. 4,891,165 claims: “A small, metallic capsule for encapsulating radioactive materials for medical and industrial diagnostic, therapeutic and functional applications, comprising: at least first and second metallic sleeves, each of said sleeves comprising a bottom portion having a circumferential wall extending therefrom, and having an open and opposite said bottom portion; wherein said first sleeve has an outer surface which is complementary to and substantially the same size as the inner surface of said second sleeve, said second sleeve fitting snugly over the open end of said first sleeve, thereby forming a substantially sealed, closed capsule, having an inner cavity, with substantially uniform total wall thickness permitting substantially uniform radiation therethrough.” Such slidably enaged sleeves may comprise the container 12 of this invention.

U.S. Pat. No. 5,405,309 claims: “A seed for implantation into a tumor within a living body to emit X-ray radiation thereto comprising at least one pellet of an electroconductive support substantially non-absorbing of X-rays, having electroplated thereon a layer of a palladium composition consisting of carrier-free palladium 103 having added thereto palladium metal in an amount sufficient to promote said electroplating, said at least one electroplated pellet containing Pd-103 in an amount sufficient to provide a radiation level measured as apparent mCi of greater than 0.5, and a shell of a bicompatible material encapsulating said at least one electroplated pellet, said biocompatible material being penetrable by X-rays in the 20-23 kev range.”

In one preferred embodiment, and referring to FIG. 1A, the assembly 10 is preferably comprised of a shield 35 that is adapted to prevent radiation from escaping from assembly 10 when such shield is in a first position, and to allow radiation to escape from assembly 10 when such shield is in a second position. It should be recognized that the depiction in FIG. 1A is merely a schematic one that does not necessarily accurately illsustrate the size, scale, shape, or functioning of the shield 35.

One may use prior art radiation shields as shield 35 to effectuate such a selective delivery of radiation from radioactive material 33. Thus, by way of illustration, and referring to U.S. Pat. No. 5,213,561 (the entire disclosure of which is hereby incorporated by reference into this specification), the shield 35 may comprise “shielding means” that comprises “. . . a retractable sleeve around said radioactive source, said sleeve being selectively movable relative to said source to expose said source when said source has been positioned at said site . . . ” (see claim 1 of U.S. 5,213,561). Such claim 1 of U.S. Pat. No. 5,213,561, in its entirey describes: “A device for reducing the incidence of restenosis at a site within a vascular structure following percutaneous transluminal coronary or peripheral angioplasty of said site, comprising, an elongated flexible member which is insertable longitudinally through vascular structure, an intravascular radioactive source mounted at a distal end of said flexible member, said source being positionable at an intravascular angioplasty site within said vascular structure for radiating said site by inserting said flexible member longitudinally through said structure, radiation shielding means on said flexible member for selectively shielding and exposing said radioactive source, said shielding means being a retractable sleeve around said radioactive source, said sleeve being selectively movable relative to said source to expose said source when said source has been positioned at said site, thereby to radiate said site, said flexible member, source and shielding means having dimensions sufficiently small that said device is insertable longitudinally through said vascular structure.”

As is disclosed in U.S. Pat. No. 5,213,561, “FIG. 1 of the drawings shows a balloon catheter guidewire 1 which can be inserted through the center of a balloon catheter for steering the catheter through vascular structure to a site where an angioplasty is to be performed. The guidewire 1 has an outer sleeve 3 around an inner or center wire 5. The guidewire structure 1 is sized to fit within a balloon catheter tube to allow guidance or steering of the balloon catheter by manipulation of guidewire 1. The outer sleeve 3 of the guidewire is preferably a tightly wound wire spiral or coil of stainless steel, with an inside diameter large enough so that it can be slid or shifted longitudinally with respect to the inner wire 5. The distal end 7 of inner wire 5 is the portion of the guidewire 1 which is to be positioned for radiation treatment of the site of the angioplasty. The distal end 7 has a radioactive material 9 such as Cobalt-60 which provides an intravascular radiation source, that is, it can be inserted through the vascular structure and will irradiate the site from within, as distinguished from an external radiation source. Outer sleeve 3 has an end portion 11 at its distal end which is made of or coated with a radiation shielding substance for shielding the radioactive source 9. In a preferred embodiment, the shielding section is lead or lead coated steel. The remaining portion 13 of the outer sleeve 3, extending from shielding section 11 to the other end of guidewire 1 (opposite from distal end 7) can be of a non-shielding substance such as stainless steel wire. By way of example, the guidewire may for example be 150 cm. long with an 0.010″ inner wire, having a 30 mm. long radioactive end 9, and a sleeve 3 of 0.018″ diameter having a lead coating 11 which is 30 cm. long. Except for the radioactive source 9 and retractable shielding 11 at the tip, guidewire 1 may be generally conventional. As already noted, the outer sleeve 3 of the guidewire 1 is slidable over the inner wire 5, at least for a distance sufficient to cover and uncover radioactive material 9, so that the shielding section 11 of the outer sleeve can be moved away from the radioactive material 9 to expose the angioplasty site to radiation. After the exposure, the outer sleeve is shifted again to cover the radioactive section. Such selective shielding prevents exposure of the walls of the vascular structure when the guidewire 1 is being inserted or removed.” This first embodiment of U.S. Pat. No. 5,213,561 may be used as the shield 35 of FIG. 1A.

Referring again to U.S. Pat. No. 5,212,561, it is also disclosed that: “A second embodiment of the invention, as shown in FIG. 2, includes a balloon catheter 15. The balloon catheter 15 has a balloon 19 at its distal end 21 and is constructed of a medically suitable plastic, preferably polyethylene. Catheter 15 has a center core or tube 17 in which a conventional guidewire 23 is receivable. Particles or crystals of radioactive material 25 (which again may be Cobalt-60) are embedded in or mounted on tube 17 inside balloon 19. A retractable radiation shielding sleeve 27 is slidable along tube 17 and covers source 25, blocking exposure to radiation, until it is shifted away (to the left in FIG. 2). Upon completion of angioplasty, the shielding sleeve 27 is retracted and the area of the injury is irradiated. Such structure allows radiation of the vascular structure immediately following completion of angioplasty, without separately inserting a radiation source. This “second embodiment” of U.S. Pat. No. 5,213,561 also may be used in as the shield 35 of FIG. 1A.

Thus, by way of further illustration, and referring to U.S. Pat. No. 5,498,227 (the entire disclosure of which is hereby incorporated by reference into this specification), one may use an “ . . . outer layer disposed about said inner core for attenutating the radiation provided by said inner core . . .” (see claim 1 of U.S. Pat. No. 5,498,227).

Thus, and by way yet of further illustration, and referring to U.S. Pat. No. 5,605,530 (the entire disclosure of which is hereby incorporated by reference into this specification), the radiation shield 35 may be “ . . . a generally cylindrical radiation shield 20 . . . .”

Thus, by way of further illustration, and referring to U.S. Pat. No. 6,196,963 (the entire disclosure of which is hereby incorporated by reference into this specification), one may use “ . . . a proximal distal portion which is adapted to substantially prevent radiation from transmitting radially from the radiation passageway . . . ” (see claim 4 of such patent).

As is disclosed at column 20 of U.S. Pat. No. 6,196,963, the radiation shield 35 may be made of material “ . . . which is substantially radiopaque, such as for example . . . tantalum, gold, tungsten, lead, or lead-loaded borosilicate materials.”

One means for selectively delivering radiation from the assembly of U.S. Pat. No. 6,196,963 is disussed at column 22 of such patent, and these means may be used as shield 35. It is disclosed in such column 22 that: : “It is to be further appreciated by view of FIG. 1 and by reference to the description above that radiation member (20) is delivered to the in vivo site through second delivery member (40), as just described, by means of first delivery member (30). This may be accomplished according to many different modes of using the beneficial features of the invention shown in FIG. 1. One specific mode is herein provided however for the purpose of further illustration. According to this specific mode of using the assembly shown in FIG. 1, proximal passageway (16) is aligned with storage chamber (13) while distal passageway (18) is left out of alignment with storage chamber (13), thereby opening the first proximal window at the proximal cap and maintaining the second distal window relative to the storage chamber (13) at the distal cap (17). First delivery member (30) is then advanced within the storage chamber (13) through the first, proximal window, forcing radiation member (20) distally within storage chamber (13) until a force may be exerted with first delivery member (30) onto radiation member (20) to allow interlocking engagement of the two members. With the proximal delivery coupler (49) of second delivery member (40) engaged to body coupler (19), distal cap (17) is then adjusted to align distal passageway (18) with storage chamber (13), thereby adjusting the second, distal window to its respective open position relative to storage chamber (13). First delivery member (30) may then be advanced distally to force radiation member (20) out of storage chamber (13) and into second delivery member (40). It is to be further appreciated that distal end portion (43) of second delivery member (40) will be positioned at the desired brachytherapy location before engaging radiation member (20) and first delivery member (30) within its internal delivery lumen. Moreover, the distal location which the internal delivery lumen (not shown) terminates in second delivery member (40) may be a closed terminus or may be open, such as through a distal port (not shown) at the tip of second delivery member (40) although a closed terminus is preferred. In the variation where the distal location is a closed terminus, radiation member (20) may be completely isolated from intimate contact with body tissues, such as blood, and may therefore be recoverable post-procedure and reused in subsequent procedures. In this embodiment, however, second delivery member (40) may require further adaptation for positioning at the desired brachytherapy site, such as including a separate guidewire lumen adapted to track over a guidewire, or adapting second delivery member (40) to be controllable and steerable, such as having a shapeable/deflectable and torqueable tip, or adapting second delivery member (40) to slideably engage within another delivery lumen of yet a third delivery device positioned within the desired site. On the other hand, where the distal location of the internal delivery lumen is open at a distal port, the second delivery member (40) may be trackable over a guidewire engaged within the internal delivery lumen, and the guidewire may be simply removed after positioning, and replaced with the radiation member (20) and first delivery member (30). However, the “blood isolation” and therefore radiation member re-use benefits of the first, closed terminus variation are lost in a trade-off with the multi-functional aspects of the “open port” second variation, and therefore the radiation member may not be reuseable in this mode for the second delivery member.”

By way of yet futher illustration, and referring to U.S. Pat. No. 6,338,709, the selective shield 35 may be, e.g., “ . . . a sheath for shielding the vessel from radiation when the segement is not being treated . . .” (see, e.g., claim 13). In the device of U.S. Pat. No. 6,338,709, a radiation source disposed within a balloon is shielded when the balloon is not inflated but exposes the vessel walls when the balloon is inflated; such a device, e.g., may be disposed in container 12 (see FIG. 1 of the instant case).

By way of yet further illustration, and referring to U.S. Pat. No. 6,471,631 (the entire disclosure of which is hereby incorporated by reference into this specification), one may use within the container 12 (and as a shield 35) “ . . . control means inside said capsule for controllably altering an amount of radiation transmitted through said outer capsule . . . ” (see claim 1). In particular, there is described in claim 1 of U.S. Pat. No. 6,471,631 “An implantable radiation therapy device, comprising: a) a biocompatible outer capsule having a wall adapted to transmit radiation therethrough; b) a radioactive material located inside said outer capsule and emitting radiation; and c) control means inside said capsule for controllably altering an amount of said radiation transmitted through said outer capsule, wherein said radioactive material and said control means are irremovable from inside said capsule without opening said capsule.”

U.S. Pat. No. 6,471,631 also discloses: “Referring now to FIG. 1, a radiation therapy seed 10 according to the invention is shown. The seed 10 includes an inner capsule 12, preferably made from a radiopaque material, such as lead, provided within a biocompatible outer capsule 14, preferably made from titanium, aluminum, stainless steel, or another substantially radiotranslucent material. Alternatively, referring to FIG. 1A, the inner capsule may be made from a radiotranslucent material and its exterior surface 25a may be coated or other provided with, e.g., as a sleeve, a radiopaque material 24a. Furthermore, while not preferred, the radiopaque material may be provided to the interior surface 27a of the inner capsule 12a (either by deposition thereon or an internal sleeve provided thereagainst). The outer capsule 14 is sealed closed about the inner capsule 12 according to any method known in the art, including the methods disclosed in previously incorporated U.S. Ser. No. 09/133,081. For treatment of the prostate, the outer capsule preferably has a diameter of less than 0.10 inches, and more typically a diameter of less than 0.050 inches, and preferably has a length of less than 0.50 inches, and more typically a length of less than 0.16 inches.” The shielding materials described in U.S. Pat. No. 6,471,631 may be used in or on the shield 35 of the instant invention.

U.S. Pat. No. 6,471,631 also discloses “The inner capsule 12 includes first and second ends 16, 18, and respective first and second openings 20, 22 at the respective ends. The inner capsule 12 is preferably coaxially held within the outer capsule 14 at the first and second ends 16, 18 of the inner capsule 12, such that a preferably uniform space 28 is provided between the inner and outer capsules.”

U.S. Pat. No. 6,471,631 also discloses “At the first end 16, the inner capsule 12 is at least partially filled with a meltable solid radioactive material 30. The radioactive material is preferably a low temperature melting, low Z carrier in which particles 31 provided with a radioactive isotope 33 are suspended. For the carrier, a low melting point is preferably characterized by under 160° F., and more preferably under 140° F. but over 105° F., such that at room temperature and body temperature, the seed is inactive as the radioactive material is substantially contained within the radiopaque inner capsule 12. Wax is a preferred carrier, although other carriers such as certain metals and polymers may be used. Exemplar isotopes include I-125, Pd-103, Cs-131, Xe-133, and Yt-169, which emit low energy X-rays and which a have relatively short half-life.” The material 33 may, e.g., be such a “meltable solid radioactive material,” and it may be melted by the application of heat caused by the activation of the nanomagentic material by a source of external radiation (as will be discussed later in this specification).

U.S. Pat. No. 6,471,631 also discloses “A piston 32 is provided in the inner capsule 12 and, upon the liquefaction of the radiopaque material 30, is capable of moving, e.g., by sliding, along a length of the inner capsule. A spring element 34 is provided between the second end 18 of the inner capsule 12 and the piston 32, forcing the piston against the radiopaque material.” Such a piston assembly may also be used in the assembly 10 of the instant case, especially when used in conjunction of the meltable radioactive material 33 and the nanomagnetic material.

U.S. Pat. No. 6,471,631 also discloses “Turning now to FIG. 2, when it is desired to increase or initiate radiation emission by the seed, that is, ‘activate’ the seed, the seed may be ‘activated’ by applying heat which causes the radioactive material 30 to melt. The heat may be applied, for example, by hot water provided in the urethra (for seeds implanted to treat prostatic conditions), by microwave radiation, or by other types of radiation. The spring element 34 provides force against the piston 32 which, in turn, forces the radioactive material 30 out of the first openings 20 and into the space 28 between the inner and outer capsules 12, 14. The second openings 22 permit gas trapped between the inner and outer capsules 12, 14 to be moved into the inner capsule 12 as the radioactive material 30 flows and surrounds the radiopaque inner capsule 12. It will also be appreciated that second openings 22 are not required if the space 28 is evacuated during manufacture. Once the radioactive material has surrounded the inner capsule, the capsule is substantially ‘activated’.” In one preferred embodiment (see FIGS. 1 and 1A), meltable radioactive material is “activated” (i.e., melted) by the application of heat from manomagnetic material, which heat is in turn created by the “activation” of the nanomagnetic material by a source of electromagnetic radiation.

U.S. Pat. No. 6,471,631 also discloses “In a variation of the above, it will be appreciated that some radioactive particles 31 or the isotope 33 may be initially provided outside the inner capsule (on the exterior surface of inner capsule, interior surface of outer capsule, or within space 28), such that movement of the radioactive material 30 out of the inner capsule operates to increase, rather than activate, radiation emission by the seed 10.” Such a variation also may be used in the instant invention.

U.S. Pat. No. 6,471,631 also discloses “Referring now to FIG. 3, according to a second embodiment of the invention, substantially similar to the first embodiment, the radiation therapy seed 110 includes a radiopaque inner capsule (or inner cylinder) 112 provided within a radiotransparent outer capsule 114. The inner capsule 112 includes first and second ends 116, 118, and one or more openings 120 at the first end. A solid, low temperature melting, radioactive material 130 is provided within the inner capsule 112. A piston 132 is provided in the inner capsule 112 against the radioactive material 130, and a pressurized fluid (liquid or gas) 134 is provided between the piston 132 and the second end 118 of the inner capsule urging the piston toward the first end 116. Turning now to FIG. 4, the seed 110 may be ‘activated’ by applying heat energy which causes the radioactive material 130 to melt. The pressurized fluid 134 then moves the piston 132 away from the second end 118, and the piston 132 moves the melted radioactive material 130 through the first openings 120 in the inner capsule into the space 128 between the inner capsule 112 and the outer capsule 114. Flow of the radioactive material 130 such that the radioactive material surrounds the inner capsule 112 is thereby facilitated.” This “second embodiment” of U.S. Pat. No. 6,471,631 may be utilized in the instant invention, wherein the radioactive material is melted by heat derived from the nanomagentic material.

U.S. Pat. No. 6,471,631 also discloses “Referring now to FIG. 5, according to a third embodiment of the invention, the radiation therapy seed 210 includes a capsule 214 having therein a rod 230 formed from a low melting point radioactive material which is provided with an elastic cover 244, e.g., latex, stretched thereover. Alternatively, the cover may be made from a heat shrinkable material. The cover 244 is provided with a radiopaque coating 226 thereon. The rod 230 and cover 244 preferably substantially fill the interior 246 of the capsule 214. As such, radiation emission is limited to the ends 248 of the rod. Turning now to FIG. 6, when the capsule 214 is heated, the rod 230 liquefies and the cover 244 collapses inward to force the radioactive material out from within the cover. The radioactive material 230 thereby surrounds the collapsed cover 244, with radiopaque material 226 deposited thereon, and increases the radioactive emission by the seed 210.” This “third embodiment” of U.S. Pat. No. 6,471,631 may also be used in assembly 10, especially when the road 230 is caused to melt by the application of heat derived from the nanomagnetic material.”

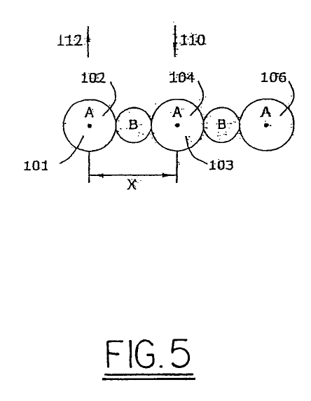

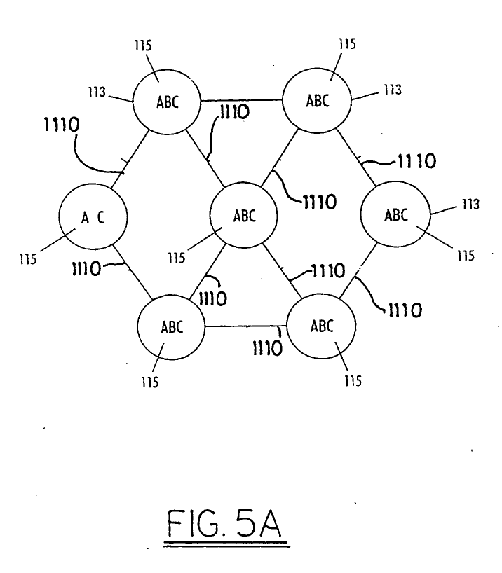

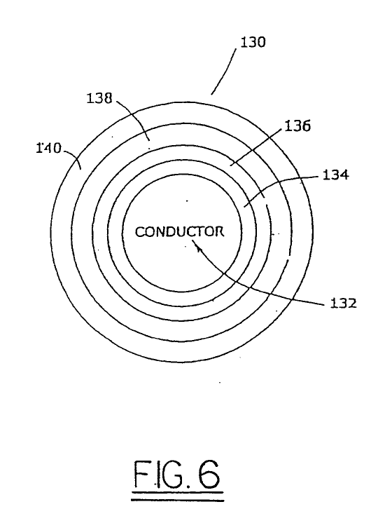

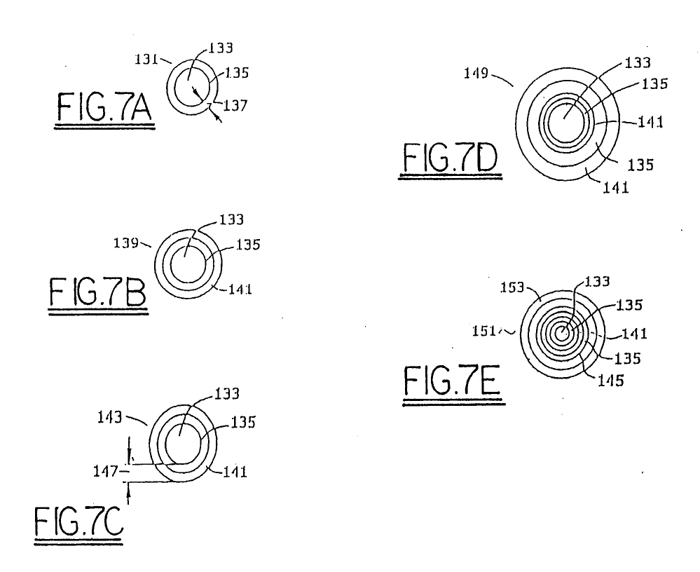

U.S. Pat. No. 6,471,631 also discloses “Referring now to FIG. 7, according to a fourth embodiment of the invention, the radiation therapy seed 310 includes an inner capsule 312 provided within an outer capsule 314. The inner capsule 312 includes first and second ends 316, 318. The first end 316 includes openings 320. A high Z material 326 is deposited on a surface 324 of the inner capsule 312. Alternatively, the inner capsule is made from a high Z material. The inner capsule is preferably coaxially held within the outer capsule, and preferably a vacuum is provided therebetween. The inner capsule 312 is partially filled with a radioactive material 330 which is liquid at body temperature, e.g., a dissolved radioactive compound. The inner capsule is also provided with a pressurized fluid (gas or liquid) 334. A piston 332 separates the radioactive material 330 and the pressurized fluid 334. The liquid material 330 is contained within the inner capsule by a wax plug 346 or the like, which is substantially solid at body temperature and which blocks the passage of the liquid radioactive material 330 through the openings 320 at the first end 316 of the inner capsule 312. Turning now to FIG. 8, when the seed 310 is heated, the plug 346 is melted and the pressurized fluid 334 forces the melted plug 346 and radioactive material 330 to exit the openings 320 at the first end 316 of the inner capsule 312 and surround the inner capsule and high Z material 326 thereof such that radiation may be emitted by the seed.” This fourth embodiment of U.S. Pat. No. 6,471,631 may also be used in conjunction with the assembly 10 of FIG. 1, especially using the nanomagnetic material to heat the plug 346.

U.S. Pat. No. 6,471,631 also discloses “It will be appreciated that as an alternative to a wax plug 346 or the like, a frangible disc or valve may be utilized to retain the liquid radioactive material. The disc or valve may be operated via heat or mechanical means to controllably permit the radioactive material to flow out of the inner capsule.” One may use the nanomagnetic material to activate the “frangible disc or valve”.

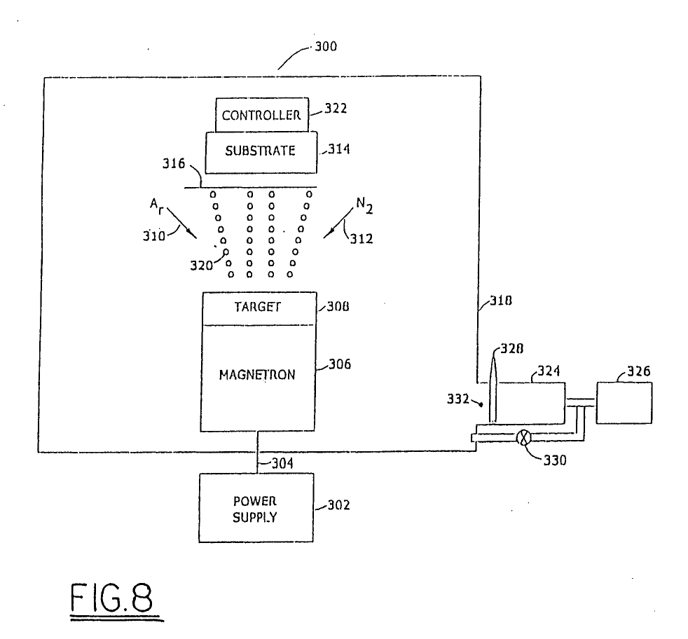

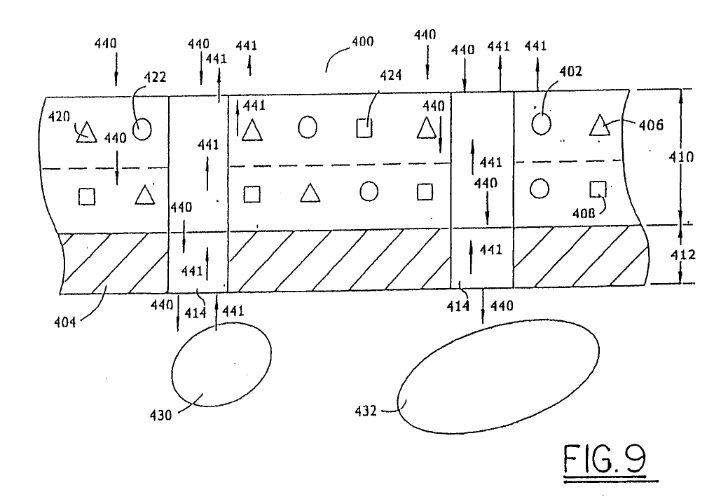

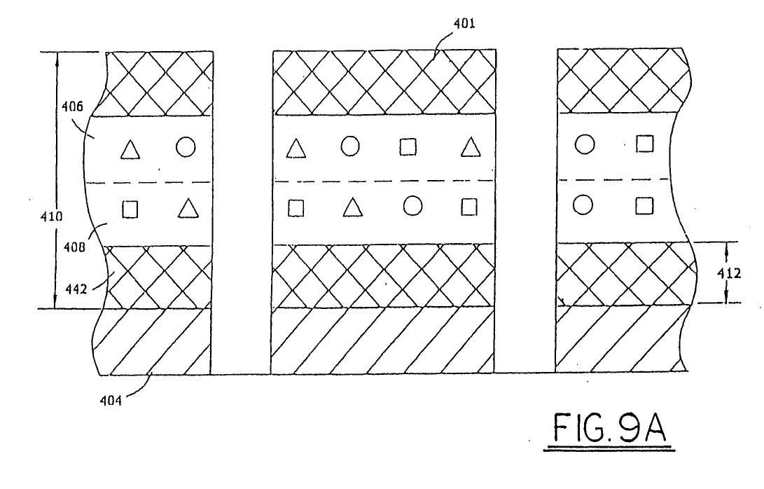

U.S. Pat. No. 6,471,631 also discloses “Referring now to FIG. 9, according to a fifth embodiment of the invention, the radiation therapy seed 410 includes an inner capsule 412 provided within an outer capsule 414. The inner capsule 412 is preferably held substantially coaxial within the outer capsule by a gas permeable tube 448, e.g., a mesh or perforate tube formed of a low Z metal or plastic. The inner capsule 412 is comprised of first and second preferably substantially tubular components 450, 452, each having a closed end 454, 456, respectively, and an open end 458, 460, respectively. The open end 458 of the first component 450 is sized to receive therein at least the open end 460 and a portion of the second component 452. The first and second components 450, 452 together thereby form a “closed” inner capsule 412. At least one of the first and second components is provided with a hole 462 which is blocked by the other of the first and second components when the inner capsule is in the “closed” configuration. A gas 434 is provided in the closed inner capsule 412. The first component and second components 450, 452 are made from a substantially low Z material. The second component 452 is provided with a plurality of preferably circumferential bands 464 of a radioactive material, while the first component 450 is provided with a plurality of preferably circumferential bands 466 of a high Z material. The first and second components are fit and aligned together such that along the length of the inner capsule 412 a series of bands in which the radioactive material 464 is covered by the high Z material 466 are provided. The bands 466 of high Z material substantially block the transmission of radiation at the isotope bands 464. Turning now to FIG. 10, when the seed 410 is heated, the gas 434 within the inner capsule 412 increases in pressure and forces the second component axially away from the first component such that the volume of the inner capsule increases. As the first and second components 450, 452 move axially apart, the hole 462 becomes exposed which equalizes the pressure between the interior of the inner capsule 412 and the interior of the outer capsule 414, terminating the axial movement. The hole 462 is preferably positioned such that movement is terminated with the high Z bands 466 of the first component 450 substantially alternating with the radioactive isotope bands 464 of the second component 452, such that the seed is activated for radiation emission.” One may use this embodiment with regard to applicants' assembly 10 and heat the seed 410 with the nanomagnetic material.

U.S. Pat. No. 6,471,631 also discloses “It will be appreciated that the other means may be used to move the first and second components 450, 452 relative to each other. For example, a one-way inertial system or an electromagnetic system may be used. In addition, it will be appreciated that the inner capsule 412 may be configured such that the high Z bands 466 initially only partially block the radioactive isotope bands 464; i.e., that the seed 410 may be activated from a first partially activate state to a second state with increased radioactive emission.” One may use such “ . . . other means to move the first and second compartments relative to each other . . .” in, e.g., the device of FIG. 1A.

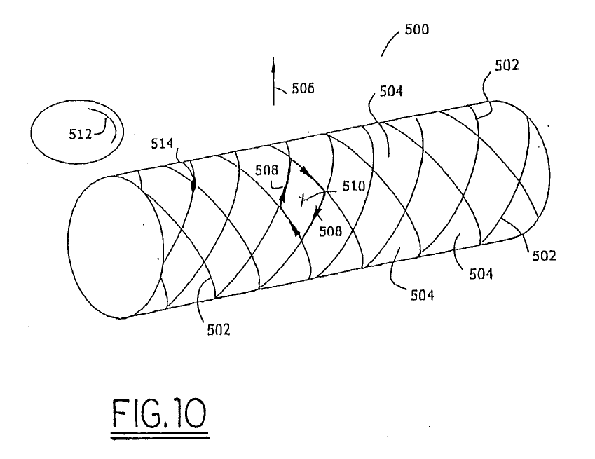

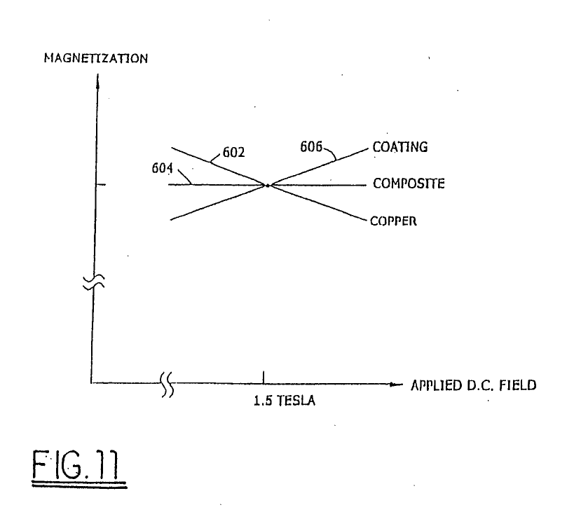

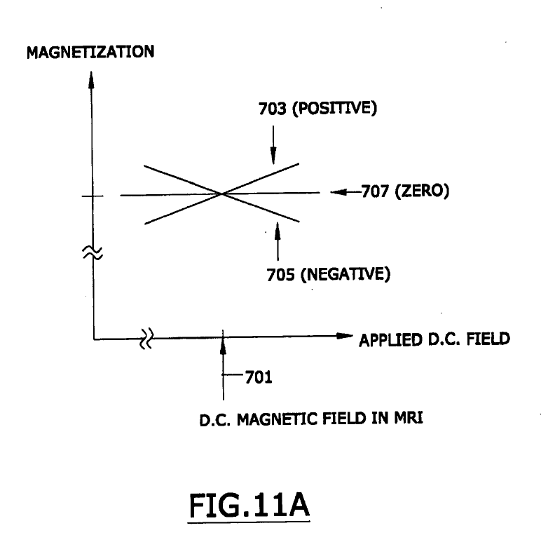

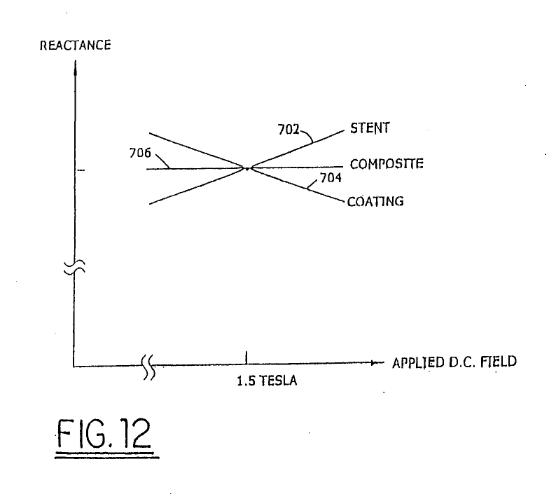

U.S. Pat. No. 6,471,631 also discloses “Referring now to FIG. 11, according to a sixth embodiment of the invention, a radiation therapy seed 610 includes an inner wire 612 provided with a circumferential band 676 of radioactive isotope material. A close wound shape memory spring coil 678 is positioned centrally over the inner wire 612 over the band 676 of radioactive material. The shape memory coil 678 is preferably made from a relatively high Z material, e.g., Nitinol, and is trained to expand when subject to a predetermined amount of heat. Second and third spring coils 680, 682 are positioned on either side of the shape memory coil 678 to maintain the high Z coil 687 at the desired location. Washers 684 may be positioned between each of the coils 678, 680, 682 to maintain the separation of the coils; i.e., to prevent the coils from entangling and to better axially direct their spring forces. The wire 612 and coils 678, 680, 682 are provided in an outer capsule 614. Turning now to FIG. 12, when the seed 610 is subject to a predetermined amount of heat, the shape memory coil 678 expands to substantially expose the isotope band 676 and to thereby activate the seed.” One may use this “sixth embodiment” in applicants' assembly 10 and use the heat from the nanomagnetic material to activate the shape memory coil 678.

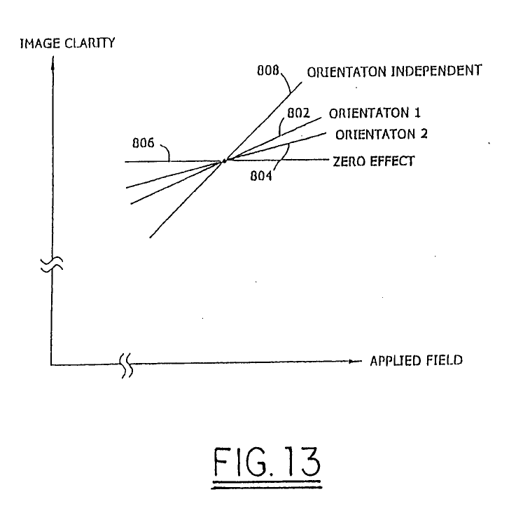

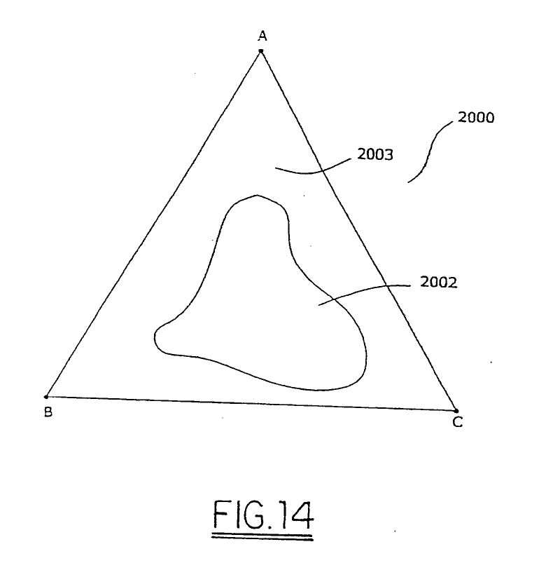

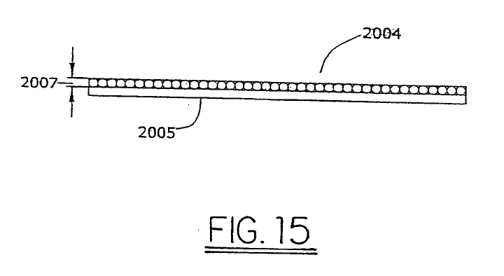

U.S. Pat. No. 6,471,631 also discloses “Referring now to FIG. 13, according to a seventh embodiment of the invention, a radiation therapy seed 710 includes a relatively radiotranslucent capsule 714 provided with preferably six rods 786 oriented longitudinally in the capsule 714. The rods 786 are made from a shape memory material which preferably is substantially radiopaque, e.g., a nickel titanium alloy. Each end of each rod is provided with a twisted portion 787. In addition, the ends of the rods are secured, e.g., by glue 789 or weld, in the outer capsule 714. When the rods are subject to heat energy, the rods are adapted to untwist at their respective twisted portions 787 about their respective axes. The rods 786 are each provided with a longitudinal stripe 788 (preferably extending about 60° to 120° about the circumference of the rods) of a radioactive isotope along a portion of their length, and preferably oriented in the capsule 714 such that the stripe 788 of each is directed radially inward toward the center C of the capsule with the high Z material of the rod substantially preventing or limiting transmission of radiation therethrough Turning now to FIG. 14, when subject to heat energy, the shape memory rods 786 within the seed 710 twist (or rotate) along their axes. The rods 786 are preferably oriented such that adjacent rods rotate in opposite directions. Turning now to FIG. 15, the rods 786 are trained to rotate preferably 180° about their respective axes. As a result, the isotope stripe 788 along each of the rods 786 is eventually directed radially outward to activate radiation emission by the seed. It will be appreciated that the rods 786 are not required to be substantially radiopaque and that alternatively, or additionally, the rods may be circumferentially deposited with a relatively high Z material along their length at least diametrically opposite the longitudinal stripes of radioactive isotopes, and preferably at all locations on the rods other than on the stripes 788. Furthermore, it will be appreciated that fewer than six or more than six rods may be provided in the capsule. Moreover, a central rod may also be used to maintain the rods in the desired spaced apart configuration; i.e., such that the rods together form a generally circular cross section . . . . ” This “seventh embodiment” of U.S. Pat. No. 6,471,631 may also be used in applicants' assembly 10, and the rods 786 may be activated by heat from the nanomagnetic material.

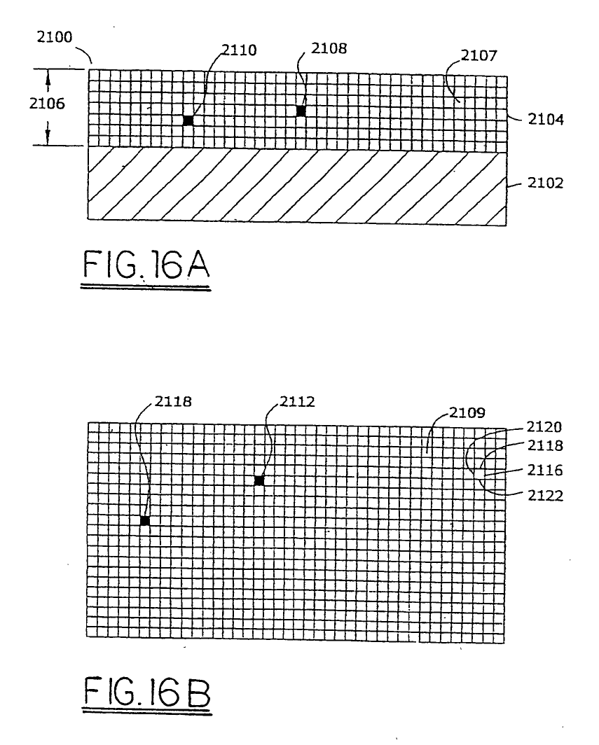

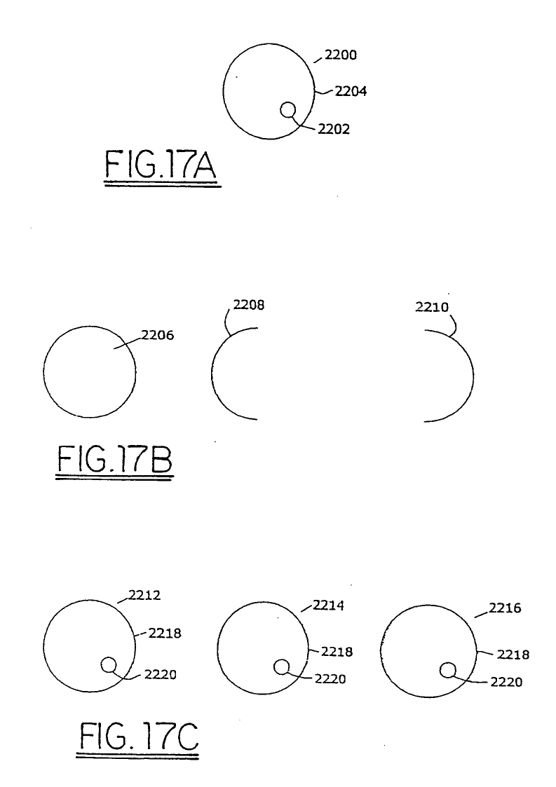

U.S. Pat. No. 6,471,631 also discloses that: “Referring now to FIG. 16, according to an eighth embodiment of the invention, a radiation therapy seed 810 includes a relatively radiotranslucent capsule 814 provided with preferably three elongate shape memory strips 890 positioned lengthwise in the capsule 814. It will be appreciated that two or four or more strips 890 may also be used. The strips are preferably made from Nitinol and are also preferably coated with a high Z material 891, e.g., gold or a heavy metal, on one side (an initially outer side), and with a radioactive isotope 892 on the side opposite the high Z material (an initially inner side). The strips 890 are preferably positioned in the capsule at 120° relative separation. The configuration of the strips 890 and the high Z material on the outer side of the strips substantially limits radiation emission by the seed, as radiation is emitted only from between the ends of the strips, at 896. The shape memory strips 890 are trained to bend. As shown in FIGS. 17 through 19, when heat is applied to the seed, the strips 890 fold into their bent configuration such that eventually the radioactive material 892 of the strips 890 is located substantially on an exterior surface of the strips, while the high Z material is located on an interior side of the strips to further activate the seed. The strips 890 may be coupled to the capsule 814 by posts (not shown) to maintain their relative positions during bending.” These “shape memory strips 890” may also be used in applicants' assembly 10, and the nanomagnetic material may be used to activate such memory strips 890.

By way of yet further illustration, and referring to U.S. Pat. No. 6,585,633 (the entire disclosure of which is hereby incorporated by reference into this specification),. the shield 35 may be “ . . . a radiaton shield slideablly disposed around said cartridge body.” claim 1 of this patent describes: “A seed cartridge assembly comprising: a cartridge body; a seed drawer slideably disposed within said cartridge body; a radiation shield slideably disposed around said cartridge body; and a seed retainer in said seed drawer, wherein the seed cartridge assembly can be autoclaved without destroying the assembly's dimensions and said cartridge body includes a transparent or translucent viewing lens.”

Referring again to FIGS. 1 and 1A, and to the preferred embodiment depicted therein, the seed assembly 10 is preferably comprised of a polymeric material 14 disposed above the sealed container 12. In the embodiment depicted in FIG. 1, the polymeric material 14 is contiguous with a layer 16 of magnetic material. In another embodiment, not shown in FIG. 1, the polymeric material 14 is contiguous with the sealed container 12.

The polymeric material 14 is preferably comprised of one or more therapeutic agents 18, 20, 22, 24, 26, 28, and/or 30 that are adapted to be released from the polymeric material 14 when the assembly 10 is disposed within a biological organism. The polymeric material 14 may be, e.g., any of the drug eluting polymers known to those skilled in the art.

By way of illustration, and referring to U.S. Pat. No. 3,279,996 (the entire disclosure of which is hereby incorporated by reference into this specification), the polymeric material 14 may be silicone rubber; such silicone rubber may be used as the material 14. This patent claims “An implantate for releasing a drug in the tissues of a living organism comprising a drug enclosed in a capsule of silicone rubber, . . . said drug being soluble in and capable of diffusing through said silicone rubber to the outer surface of said capsule . . . .” One may use, as, e.g., therapeutic agent 18, a material that is soluble in and capable of diffusing through the polymeric material 14.

At column 1 of U.S. Pat. No. 3,279,996, other “carrier agents” which may be used as polymeric material 14 are also disclosed, including “ . . . beeswax, peanut oil, stearates, etc.” Any of these “carrier agents” may be used as the polymeric material 14.

By way of further illustration, and as is disclosed in U.S. Pat. No. 4,191,741 (the entire disclosure of which is hereby incorporated by reference into this specification), one may use dimethylpolsiloxane rubber as the polymeric material 14. This patent claims “A solid, cylindrical, subcutaneous implant for improving the rate of weight gain of ruminant animals which comprises (a) a biocompatible inert core having a diameter of from about 2 to about 10 mm. and (b) a biocompatible coating having a thickness of from about 0.2 to about 1 mm., the composition of said coating comprising from about 5 to about 40 percent by weight of estradiol and from about 95 to about 60 percent by weight of a dimethylpolysiloxane rubber.” One may use estradiol as a therapeutic agent (e.g., agent 18) disposed within polymeric material 14.

In column 1 of U.S. Pat. No. 4,191,741, other materials which may be used as polymeric material 14 are disclosed. Thus, it is stated in such patent that “Long et al. U.S. Pat. No. 3,279,996 describes an implant for releasing a drug in the tissues of a living organism comprising the drug enclosed in a capsule formed of silicone rubber. The drug migrates through the silicone rubber wall and is slowly released into the living tissues. A number of biocompatible silicone rubbers are described in the Long et al. patent. When a drug delivery system such as that described in U.S. Pat. No. 3,279,996 is used in an effort to administer estradiol to a ruminant animal a number of problems are encountered. For example, an excess of the drug is generally required in the hollow cavity of the implant. Also, it is difficult to achieve a constant rate of administration of the drug over a long time period such as from 200 to 400 days as would be necessary for the daily administration of estradiol to a growing beef animal. Katz et al. U.S. Pat. No. 4,096,239 describes an implant pellet containing estradiol or estradiol benzoate which has an inert spherical core and a uniform coating comprising a carrier and the drug. The coating containing the drug must be both biocompatible and biosoluble, i.e., the coating must dissolve in the body fluids which act upon the pellet when it is implanted in the body. The rate at which the coating dissolves determines the rate at which the drug is released. Representative carriers for use in the coating material include cholesterol, solid polyethylene glycols, high molecular weight fatty acids and alcohols, biosoluble waxes, cellulose derivatives and solid polyvinyl pyrrolidone.” The polymeric material 14 used in the device 10 of FIG. 1 is, in one embodiment, both biocompatible and biosoluble.

By way of yet further illustration, and referring to U.S. Pat. No. 4,429,080 (the entire disclosure of which is hereby incorporated by reference into this specification), the polymeric material 14 may be a synthetic absorbable copolymer formed by copolymerizing glycolide with trimethylene carbonate. This material may be used as the polymeric material 14.

By way of yet further illustration, and referring to U.S. Pat. No. 4,581,028 (the entire disclosure of which is hereby incorporated by reference into this specification), the polymeric material 14 may be selected from the group consisting of polyester (such as Dacron), polytetrafluoroethylene, polyurethane silicone-based material, and polyamide. The polymeric material of this patent is comprised “ . . . of at least one antimicrobial agent selected from the group consisting of the metal salts of sulfonamides.” In one embodiment, the polymeric material 14 is comprised of an antimicrobial agent.

By way of yet further illustration, and referring to U.S. Pat. No. 4,481,353, (the entire disclosure of which is hereby incorporated by reference into this specification), the polymeric material 14 may be the bioresorbable polyester disclosed in such patent. U.S. Pat. No. 4,481,353 claims “A bioresorbable polyester in which monomeric subunits are arranged randomly in the polyester molecules, said polyester comprising the condensation reaction product of a Krebs Cycle dicarboxylic acid or isomer or anhydride thereof, chosen for the group consisting of succinic acid, fumaric acid, oxaloacetic acid, L-malic acid, and D-malic acid, a diol having 2, 4, 6, or 8 carbon atoms, and an alpha-hydroxy carboxylic acid chosen from the group consisting of glycolic acid, L-lactic acid and D-lactic acid.” The polymeric material 14 may be a bioresorbable polyester.

By way of yet further illustration, and referring to U.S. Pat. No. 4,846,844 (the entire disclosure of which is hereby incorporated by reference into this specification), the polymeric material 14 may be a silicone polymer matrix in which an anabolic agent (such as an anabolic steroid, or estradiol) is disposed. This patent claims “An implant adapted for the controlled release of an anabolic agent, said implant comprising a silicone polymer matrix, an anabolic agent in said polymer matrix, and an antimicrobial coating, wherein the coating comprises a first-applied non-vulcanizing silicone fluid and a subsequently applied antimicrobial agent in contact with said fluid.” The therapeutic agent (such as agent 18) may be an anabolic agent; and the polymeric material may be a silicone polymer.

By way of yet further illustration, and referring to U.S. Pat. No. 4,916,193 (the entire disclosure of which is hereby incorporated by reference into this specification), the polymeric material 14 may be a copolymer containing carbonate repeat units and ester repeat units (see, e.g., claim 1 of the patent). As disclosed in column 2 of the patent, it may also be “collagen,” “homopolymers and copolymers of glycolic acid and lactic acid,” “alpha-hydroxy carboxylic acids in conjunction with Krebs cycle dicarboxylic acids and aliphatic diols,” “polycarbonate-containing polymers,” and “high molecular weight fiber-forming crystalline copolymers of lactide and glycolide.” Thus, it is disclosed in such column 2 that: “Various polymers have been proposed for use in the fabrication of bioresorbable medical devices. Examples of absorbable materials used in nerve repair include collagen as disclosed by D. G. Kline and G. J. Hayes, “The Use of a Resorbable Wrapper for Peripheral Nerve Repair, Experimental Studies in Chimpanzees”, J. Neurosurgery 21, 737 (1964). Artandi et al., U.S. Pat. No. 3,272,204 (1966) reports the use of collagen protheses that are reinforced with nonabsorbable fabrics. These articles are intended to be placed permanently in a human body. However, one of the disadvantages inherent with collagenous materials, whether utilized alone or in conjunction with biodurable materials, is their potential antigenicity. Other biodegradable polymers of particular interest for medical implantation purposes are homopolymers and copolymers of glycolic acid and lactic acid. A nerve cuff in the form of a smooth, rigid tube has been fabricated from a copolymer of lactic and glycolic acids [The Hand; 10 (3) 259 (1978)]. European patent application No. 118-458-A discloses biodegradable materials used in organ protheses or artificial skin based on poly-L-lactic acid and/or poly-DL-lactic acid and polyester or polyether urethanes. U.S. Pat. No. 4,481,353 discloses bioresorbable polyester polymers, and composites containing these polymers, that are also made up of alpha-hydroxy carboxylic acids, in conjunction with Krebs cycle dicarboxylic acids and aliphatic diols. These polyesters are useful in fabricating nerve guidance channels as well as other surgical articles such as sutures and ligatures. U.S. Pat. Nos. 4,243,775 and 4,429,080 disclose the use of polycarbonate-containing polymers in certain medical applications, especially sutures, ligatures and haemostatic devices. However, this disclosure is clearly limited only to “AB” and “ABA” type block copolymers where only the “B” block contains poly(trimethylene carbonate) or a random copolymer of glycolide with trimethylene carbonate and the “A” block is necessarily limited to glycolide. In the copolymers of this patent, the dominant portion of the polymer is the glycolide component. U.S. Pat. No. 4,157,437 discloses high molecular weight, fiber-forming crystalline copolymers of lactide and glycolide which are disclosed as useful in the preparation of absorbable surgical sutures. The copolymers of this patent contain from about 50 to 75 wt. % of recurring units derived from glycolide.” The polymeric material 14 may be one or more of the copolymers of U.S. Pat. No. 4,916,193.

By way of further illustration, and referring to U.S. Pat. No. 5,176,907 (the entire disclosure of which is hereby incorporated by reference into this specification), the polymeric material 14 may be the poly-phosphoester-urethane) described and claimed in claim 1 of such patent. Furthermore, the polymeric material 14 may be one or more of the biodegradable polymers discussed in columns 1 and 2 of such patent. As is disclosed in such columns 1 and 2: “Polymers have been used as carriers of therapeutic agents to effect a localized and sustained release (Controlled Drug Delivery, Vol. I and II, Bruck, S. D., (ed.), CRC Press, Boca Raton, Fla., 1983; Leong, et al., Adv. Drug Delivery Review, 1: 199, 1987). These therapeutic agent delivery systems simulate infusion and offer the potential of enhanced therapeutic efficacy and reduced systemic toxicity.” The polymeric material may be such a poly-phosphoester-urethane.

U.S. Pat. No. 5,176,907 also discloses “For a non-biodegradable matrix, the steps leading to release of the therapeutic agent are water diffusion into the matrix, dissolution of the therapeutic agent, and out-diffusion of the therapeutic agent through the channels of the matrix. As a consequence, the mean residence time of the therapeutic agent existing in the soluble state is longer for a non-biodegradable matrix than for a biodegradable matrix where a long passage through the channels is no longer required. Since many pharmaceuticals have short half-lives it is likely that the therapeutic agent is decomposed or inactivated inside the non-biodegradable matrix before it can be released. This issue is particularly significant for many bio-macromolecules and smaller polypeptides, since these molecules are generally unstable in buffer and have low permeability through polymers. In fact, in a non-biodegradable matrix, many bio-macromolecules will aggregate and precipitate, clogging the channels necessary for diffusion out of the carrier matrix. This problem is largely alleviated by using a biodegradable matrix which allows controlled release of the therapeutic agent. Biodegradable polymers differ from non-biodegradable polymers in that they are consumed or biodegraded during therapy. This usually involves breakdown of the polymer to its monomeric subunits, which should be biocompatible with the surrounding tissue. The life of a biodegradable polymer in vivo depends on its molecular weight and degree of cross-linking; the greater the molecular weight and degree of crosslinking, the longer the life. The most highly investigated biodegradable polymers are polylactic acid (PLA), polyglycolic acid (PGA), polyglycolic acid (PGA), copolymers of PLA and PGA, polyamides, and copolymers of polyamides and polyesters. PLA, sometimes referred to as polylactide, undergoes hydrolytic de-esterification to lactic acid, a normal product of muscle metabolism. PGA is chemically related to PLA and is commonly used for absorbable surgical sutures, as is the PLA/PGA copolymer. However, the use of PGA in controlled-release implants has been limited due to its low solubility in common solvents and subsequent difficulty in fabrication of devices.” The polymeric material 14 may be a biodegradable polymeric material.

U.S. Pat. No. 5,176,907 also discloses “An advantage of a biodegradable material is the elimination of the need for surgical removal after it has fulfilled its mission. The appeal of such a material is more than simply for convenience. From a technical standpoint, a material which biodegrades gradually and is excreted over time can offer many unique advantages.”

U.S. Pat. No. 5,176,907 also discloses “A biodegradable therapeutic agent delivery system has several additional advantages: 1) the therapeutic agent release rate is amenable to control through variation of the matrix composition; 2) implantation can be done at sites difficult or impossible for retrieval; 3) delivery of unstable therapeutic agents is more practical. This last point is of particular importance in light of the advances in molecular biology and genetic engineering which have lead to the commercial availability of many potent bio-macromolecules. The short in vivo half-lives and low GI tract absorption of these polypeptides render them totally unsuitable for conventional oral or intravenous administration. Also, because these substances are often unstable in buffer, such polypeptides cannot be effectively delivered by pumping devices.”

U.S. Pat. No. 5,176,907 also discloses “In its simplest form, a biodegradable therapeutic agent delivery system consist of a dispersion of the drug solutes in a polymer matrix. The therapeutic agent is released as the polymeric matrix decomposes, or biodegrades into soluble products which are excreted from the body. Several classes of synthetic polymers, including polyesters (Pitt, et al., in Controlled Release of Bioactive Materials, R. Baker, Ed., Academic Press, New York, 1980); polyamides (Sidman, et al., Journal of Membrane Science, 7:227, 1979); polyurethanes (Maser, et al., Journal of Polymer Science, Polymer Symposium, 66:259, 1979); polyorthoesters (Heller, et al., Polymer Engineering Science, 21:727, 1981); and polyanhydrides (Leong, et al., Biomaterials, 7:364, 1986) have been studied for this purpose.” The therapeutic agent 18 may be dispersed in the polymeric material 14.

By way of yet further illustration, and referring to U.S. Pat. No. 5,194,581 (the entire disclosure of which is hereby incorporated by reference into this specification), the polymeric material 14 may the poly (phosphoester) compositons described in such patent. Furthermore, and referring again to FIG. 1, the therapeutic agents 18, 20, 22, 24, 26, 28, and/or 30 may be one or more of the drugs described at columns 6 and 7 of such patent. Referring to such columns 6 and 7, it is disclosed that: “The term “therapeutic agent” as used herein for the compositions of the invention includes, without limitation, drugs, radioisotopes, immunomodulators, and lectins. Similar substances are within the skill of the art. The term “individual” includes human as well as non-human animals.”

U.S. Pat. No. 5,194,581 also discloses “The drugs with which can be incorporated in the compositions of the invention include non-proteinaceous as well as proteinaceous drugs. The term “non-proteinaceous drugs” encompasses compounds which are classically referred to as drugs such as, for example, mitomycin C, daunorubicin, vinblastine, AZT, and hormones. Similar substances are within the skill of the art.” The therapeutic agent 18 may be such a non-proteinaceous drug.

U.S. Pat. No. 5,176,907 also discloses “The proteinaceous drugs which can be incorporated in the compositions of the invention include immunomodulators and other biological response modifiers. The term “biological response modifiers” is meant to encompass substances which are involved in modifying the immune response in such manner as to enhance the particular desired therapeutic effect, for example, the destruction of the tumor cells. Examples of immune response modifiers include such compounds as lymphokines. Examples of lymphokines include tumor necrosis factor, the interleukins, lymphotoxin, macrophage activating factor, migration inhibition factor, colony stimulating factor and the interferons. Interferons which can be incorporated into the compositions of the invention include alpha-interferon, beta-interferon, and gamma-interferon and their subtypes. In addition, peptide or polysaccharide fragments derived from these proteinaceous drugs, or independently, can also be incorporated. Also, encompassed by the term “biological response modifiers” are substances generally referred to as vaccines wherein a foreign substance, usually a pathogenic organism or some fraction thereof, is used to modify the host immune response with respect to the pathogen to which the vaccine relates. Those of skill in the art will know, or can readily ascertain, other substances which can act as proteinaceous drugs.” The therapeutic agent 18 may be such a proteinaceous drug.

U.S. Pat. No. 5,176,907 also discloses “In using radioisotopes certain isotopes may be more preferable than others depending on such factors, for example, as tumor distribution and mass as well as isotope stability and emission. Depending on the type of malignancy present come emitters may be preferable to others. In general, alpha and beta particle-emitting radioisotopes are preferred in immunotherapy. For example, if an animal has solid tumor foci a high energy beta emitter capable of penetrating several millimeters of tissue, such as 90 Y, may be preferable. On the other hand, if the malignancy consists of single target cells, as in the case of leukemia, a short range, high energy alpha emitter such as 212 Bi may be preferred. Examples of radioisotopes which can be incorporated in the compositions of the invention for therapeutic purposes are 125 I, 131 I, 90 Y, 67 Cu, 212 Bi, 211 At, 212 Pb, 47 Sc, 109 Pd and 188 Re. Other radioisotopes which can be incorporated into the compositions of the invention are within the skill in the art.” The radioactive material 33 may be comprised of alpha and/or beta particle emitting radioisotopes.

U.S. Pat. No. 5,176,907 also discloses “Lectins are proteins, usually isolated from plant material, which bind to specific sugar moieties. Many lectins are also able to agglutinate cells and stimulate lymphocytes. Other therapeutic agents which can be used therapeutically with the biodegradable compositions of the invention are known, or can be easily ascertained, by those of ordinary skill in the art.” The therapeutic agent 18 may be, e.g., a lectini.

U.S. Pat. No. 5,176,907 also discloses “Therapeutic-agent bearing” as it applies to the compositions of the invention denotes that the composition incorporates a therapeutic agent which is 1) not bound to the polymeric matrix, or 2) bound within the polymeric backbone matrix, or 3) pendantly bound to the polymeric matrix, or 4) bound within the polymeric backbone matrix and pendantly bound to the polymeric matrix. When the therapeutic agent is not bound to the matrix, then it is merely physically dispersed with the polymer matrix. When the therapeutic agent is bound within the matrix it is part of the poly(phosphoester) backbone (R′). When the therapeutic agent is pendantly attached it is chemically linked through, for example, by ionic or covalent bonding, to the side chain (R) of the matrix polymer. In the first two instances the therapeutic agent is released as the matrix biodegrades. The drug can also be released by diffusion through the polymeric matrix. In the pendant system, the drug is released as the polymer-drug bond is cleaved at the bodily tissue.” The therapeutic agent 18 may be “ . . . 1) not bound to the polymeric matrix, or 2) bound within the polymeric backbone matrix, or 3) pendantly bound to the polymeric matrix, or 4) bound within the polymeric backbone matrix and pendantly bound to the polymeric matrix . . . .”

The polymeric material 14 may be comprised of microcapsules such as, e.g., the microcapsule described in U.S. Pat. No. 6,117,455, the entire disclosure of which is hereby incorporated by reference into this specification. As is disclosed in the abstract of this patent, there is provided “A sustained-release microcapsule contains an amorphous water-soluble pharmaceutical agent having a particle size of from 1 nm-10 μm and a polymer. The microcapsule is produced by dispersing, in an aqueous phase, a dispersion of from 0.001-90% (w/w) of an amorphous water-soluble pharmaceutical agent in a solution of a polymer having a wt. avg. molecular weight of 2,000-800,000 in an organic solvent to prepare an s/o/w emulsion and subjecting the emulsion to in-water drying.” The polymeric material 14 may comprised sustained-release microcapsules of a water-soluble drug.

In one embodiment, disclosed in U.S. Pat. No. 5,484,584 (the entire disclosure of which is hereby incorporated by reference into this specification), a poly (benzyl-L-glutamate) microsphere is disclosed (see, e.g., claim 10). As is disclosed in the abstract of this patent, “The present invention relates to a highly efficient method of preparing modified microcapsules exhibiting selective targeting. These microcapsules are suitable for encapsulation surface attachment of therapeutic and diagnostic agents. In one aspect of the invention, surface charge of the polymeric material is altered by conjugation of an amino acid ester to the providing improved targeting of encapsulated agents to specific tissue cells. Examples include encapsulation of radiodiagnostic agents in 1 μm capsules to provide improved opacification and encapsulation of cytotoxic agents in 100 μm capsules for chemoembolization procedures. The microcapsules are suitable for attachment of a wide range of targeting agents, including antibodies, steroids and drugs, which may be attached to the microcapsule polymer before or after formation of suitably sized microcapsules. The invention also includes microcapsules surface modified with hydroxyl groups. Various agents such as estrone may be attached to the microcapsules and effectively targeted to selected organs.” One or more of such microspheres, comprising one or more of such targeting agents and/or radiodiagnostic agents and/or cytoxic materials, may be disposed within polymeric material 14.

As is also disclosed in U.S. Pat. No. 5,484,584, “Referring again to FIG. 1, and to the preferred embodiment depicted therein, ti will be seen that a combination of more than one therapeutic agent (such as, e.g., therapeutic agents 18 and/or 20 and/or 22 and/or 24 and/or 26 and/or 28 and/or 30) may be incorporate in to the polymeric material 14. This may be effected, e.g., by the process described in columns 7 and 8 of U.S. Pat. No. 5,194,581. As is disclosed in such patent, “A combination of more than one therapeutic agent can be incorporated into the compositions of the invention. Such multiple incorporation can be done, for example, 1) by substituting a first therapeutic agent into the backbone matrix (R′) and a second therapeutic agent by pendant attachment (R), 2) by providing mixtures of different poly(phosphoesters) which have different agents substituted in the backbone matrix (R′) or at their pendant positions (R), 3) by using mixtures of unbound therapeutic agents with the poly(phosphoester) which is then formed into the composition, 4) by use of a copolymer with the general structure [Figure] wherein m or n can be from about 1 to about 99% of the polymer, or 5) by combinations of the above.” In one embodiment, more than two therapeutic agents are incorporated into the polymeric material 14.

As is also disclosed in U.S. Pat. No. 5,484,584, “The concentration of therapeutic agent in the composition will vary with the nature of the agent and its physiological role and desired therapeutic effect. Thus, for example, the concentration of a hormone used in providing birth control as a therapeutic effect will likely be different from the concentration of an anti-tumor drug in which the therapeutic effect is to ameliorate a cell-proliferative disease. In any event, the desired concentration in a particular instance for a particular therapeutic agent is readily ascertainable by one of skill in the art.”

As is also disclosed in U.S. Pat. No. 5,484,584, “The therapeutic agent loading level for a composition of the invention can vary, for example, on whether the therapeutic agent is bound to the poly(phosphoester) backbone polymer matrix. For those compositions in which the therapeutic agent is not bound to the backbone matrix, in which the agent is physically disposed with the poly(phosphoester), the concentration of agent will typically not exceed 50 wt %. For compositions in which the therapeutic agent is bound within the polymeric backbone matrix, or pendantly bound to the polymeric matrix, the drug loading level is up to the stoichiometric ratio of agent per monomeric unit.” In one embodiment, the therapeutic agent 18 is bound to the backbone of the polymeric material 14.

Referring again to FIG. 1, the release rate(s) of therapeutic agents 18 and/or 20 and/or 22 and/or 24 and/or 26 and/or 28 and/or 30 may be varied in, e.g., the manner suggested in column 6 of U.S. Pat. No. 5,194,581. As is disclosed in such column 6, “A wide range of degradation rates can be obtained by adjusting the hydrophobicities of the backbones of the polymers and yet the biodegradability is assured. This can be achieved by varying the functional groups R or R′. The combination of a hydrophobic backbone and a hydrophilic linkage also leads to heterogeneous degradation as cleavage is encouraged, but water penetration is resisted.” As is disclosed at column 9 of such patent, “The rate of biodegradation of the poly(phosphoester) compositions of the invention may also be controlled by varying the hydrophobicity of the polymer. The mechanism of predictable degradation preferably relies on either group R′ in the poly(phosphoester) backbone being hydrophobic for example, an aromatic structure, or, alternatively, if the group R′ is not hydrophobic, for example an aliphatic group, then the group R is preferably aromatic. The rates of degradation for each poly(phosphoester) composition are generally predictable and constant at a single pH. This permits the compositions to be introduced into the individual at a variety of tissue sites. This is especially valuable in that a wide variety of compositions and devices to meet different, but specific, applications may be composed and configured to meet specific demands, dimensions, and shapes—each of which offers individual, but different, predictable periods for degradation. When the composition of the invention is used for long term delivery of a therapeutic agent a relatively hydrophobic backbone matrix, for example, containing bisphenol A, is preferred. It is possible to enhance the degradation rate of the poly(phosphoester) or shorten the functional life of the device, by introducing hydrophilic or polar groups, into the backbone matrix. Further, the introduction of methylene groups into the backbone matrix will usually increase the flexibility of the backbone and decrease the crystallinity of the polymer. Conversely, to obtain a more rigid backbone matrix, for example, when used orthopedically, an aromatic structure, such as a diphenyl group, can be incorporated into the matrix. Also, the poly(phosphoester) can be crosslinked, for example, using 1,3,5-trihydroxybenzene or (CH2 OH)4 C, to enhance the modulus of the polymer. Similar considerations hold for the structure of the side chain (R).”

By way of yet further illustration, and referring to U.S. Pat. No. 5,252,713 (the entire disclosure of which is hereby incorporated by reference into this specification), the polymeric material 14 may be a polypeptide comprising at least one drug-binding domain that non-covalently binds a drug. The means of identifying and isolating such a polypeptide is described at columns 5-7 of the patent, wherein it is disclosed that: “The process of isolating a polymeric carrier from a drug-binding, large molecular weight protein begins with the identification of a large protein that can non-covalently bind the drug of interest. Examples of such protein/drug pairs are shown in Table I. The drugs in the Table (other than the steroids) are anti-cancer drugs . . . .”

As is also disclosed in U.S. Pat. No. 5,252,713, “Other drug-binding proteins may be identified by appropriate analytical procedures, including Western blotting of large proteins or protein fragments and subsequent incubation with a detectable form of drug. Alternative procedures include combining a drug and a protein in a solution, followed by size exclusion HPLC gel filtration, thin-layer chromatography (TLC), or other analytical procedures that can discriminate between free and protein-bound drug. Detection of drug binding can be accomplished by using radiolabeled, fluorescent, or colored drugs and appropriate detection methods. Equilibrium dialysis with labeled drug may be used. Alternative methods include monitoring the fluorescence change that occurs upon binding of certain drugs (e.g., anthracyclines or analogs thereof, which should be fluorescent) . . . ”. In one detection method, drug and protein are mixed, and an aliquot of this solution (not exceeding 5% of the column volume of an HPLC column, such as a Bio-sil TSK-250 7.5×30 cm column) is loaded onto the HPLC column. The flow rate is 1 mi/min. The drug bound to protein will elute first, in a separate peak, followed by free drug, eluting at a position characteristic of its molecular weight. If the drug is doxorubicin, both a 280-nm as well as a 495-nm adsorptive peak will correspond to the elution position of the protein if interaction occurs. The elution peaks for other drugs will indicate whether drug binding occurs . . . .”

As is also disclosed in U.S. Pat. No. 5,252,713, “Knowledge of the chemical structure of a particular drug (i.e., whether chemically reactive functional groups are present) allows one to predict whether covalent binding of the drug to a given protein can occur. Additional methods for determining whether drug binding is covalent or non-covalent include incubating the drug with the protein, followed by dialysis or subjecting the protein to denaturing conditions. Release of the drug from the drug-binding protein during these procedures indicates that the drug was non-covalently bound. Usually, a dissociation constant of about 10-15 M or less indicates covalent or extremely tight non-covalent binding . . . .”

As is also disclosed in U.S. Pat. No. 5,252,713, “During dialysis, non-covalently bound drug molecules are released over time from the protein and pass through a dialysis membrane, whereas covalently bound drug molecules are retained on the protein. An equilibrium constant of about 10-5 M indicates non-covalent binding. Alternatively, the protein may be subjected to denaturing conditions; e.g., by gel electrophoresis on a denaturing (SDS) gel or on a gel filtration column in the presence of a strong denaturant such as 6M guanidine. Covalently bound drug molecules remain bound to the denatured protein, whereas non-covalently bound drug molecules are released and migrate separately from the protein on the gel and are not retained with the protein on the column.”

As is also disclosed in U.S. Pat. No. 5,252,713, “Once a protein that can non-covalently bind a particular drug of interest is identified, the drug-binding domain is identified and isolated from the protein by any suitable means. Protein domains are portions of proteins having a particular function or activity (in this case, non-covalent binding of drug molecules). The present invention provides a process for producing a polymeric carrier, comprising the steps of generating peptide fragments of a protein that is capable of non-covalently binding a drug and identifying a drug-binding peptide fragment, which is a peptide fragment containing a drug-binding domain capable of non-covalently binding the drug, for use as the polymeric carrier.”

As is also disclosed in U.S. Pat. No. 5,252,713, “One method for identifying the drug-binding domain begins with digesting or partially digesting the protein with a proteolytic enzyme or specific chemicals to produce peptide fragments. Examples of useful proteolytic enzymes include lys-C-endoprotease, arg-C-endoprotease, V8 protease, endoprolidase, trypsin, and chymotrypsin. Examples of chemicals used for protein digestion include cyanogen bromide (cleaves at methionine residues), hydroxylamine (cleaves the Asn-Gly bond), dilute acetic acid (cleaves the Asp-Pro bond), and iodosobenzoic acid (cleaves at the tryptophane residue). In some cases, better results may be achieved by denaturing the protein (to unfold it), either before or after fragmentation.”

As is also disclosed in U.S. Pat. No. 5,252,713, “The fragments may be separated by such procedures as high pressure liquid chromatography (HPLC) or gel electrophoresis. The smallest peptide fragment capable of drug binding is identified using a suitable drug-binding analysis procedure, such as one of those described above. One such procedure involves SDS-PAGE gel electrophoresis to separate protein fragments, followed by Western blotting on nitrocellulose, and incubation with a colored drug like adriamycin. The fragments that have bound the drug will appear red. Scans at 495 nm with a laser densitometer may then be used to analyze (quantify) the level of drug binding.”

As is also disclosed in U.S. Pat. No. 5,252,713, “Preferably, the smallest peptide fragment capable of non-covalent drug binding is used. It may occasionally be advisable, however, to use a larger fragment, such as when the smallest fragment has only a low-affinity drug-binding domain.”

As is also disclosed in U.S. Pat. No. 5,252,713, “The amino acid sequence of the peptide fragment containing the drug-binding domain is elucidated. The purified fragment containing the drug-binding region is denatured in 6M guanidine hydrochloride, reduced and carboxymethylated by the method of Crestfield et al., J. Biol. Chem. 238:622, 1963. As little as 20 to 50 picomoles of each peptide fragment can be analyzed by automated Edman degradation using a gas-phase or liquidpulsed protein sequencer (commercially available from Applied Biosystems, Inc.). If the peptide fragment is longer than 30 amino acids, it will most likely have to be fragmented as above and the amino acid sequence patched together from sequences of overlapping fragments.”

As is also disclosed in U.S. Pat. No. 5,252,713, “Once the amino acid sequence of the desired peptide fragment has been determined, the polymeric carriers can be made by either one of two types of synthesis. The first type of synthesis comprises the preparation of each peptide chain with a peptide synthesizer (e.g., commercially available from Applied Biosystems). The second method utilizes recombinant DNA procedures.”The polymeric material 14 may comprise one or more of the polymeric carriers described in U.S. Pat. No. 5,252,713.

As is also disclosed in U.S. Pat. No. 5,252,713, “Peptide amides can be made using 4-methylbenzhydrylamine-derivatized, cross-linked polystyrene-1% divinylbenzene resin and peptide acids made using PAM (phenylacetamidomethyl) resin (Stewart et al., “Solid Phase Peptide Synthesis,” Pierce Chemical Company, Rockford, Ill., 1984). The synthesis can be accomplished either using a commercially available synthesizer, such as the Applied Biosystems 430A, or manually using the procedure of Merrifield et al., Biochemistry 21:5020-31, 1982; or Houghten, PNAS 82:5131-35, 1985. The side chain protecting groups are removed using the Tam-Merrifield low-high HF procedure (Tam et al., J. Am. Chem. Soc. 105:6442-55, 1983). The peptide can be extracted with 20% acetic acid, lyophilized, and purified by reversed-phase HPLC on a Vydac C-4 Analytical Column using a linear gradient of 100% water to 100% acetonitrile-0.1% trifluoroacetic acid in 50 minutes. The peptide is analyzed using PTC-amino acid analysis (Heinrikson et al., Anal. Biochem. 136:65-74, 1984). After gas-phase hydrolysis (Meltzer et al., Anal. Biochem. 160: 356-61, 1987), sequences are confirmed using the Edman degradation or fast atom bombardment mass spectroscopy. After synthesis, the polymeric carriers can be tested for drug binding using size-exclusion HPLC, as described above, or any of the other analytical methods listed above.”

As is also disclosed in U.S. Pat. No. 5,252,713, “The polymeric carriers of the present invention preferably comprise more than one drug-binding domain. A polypeptide comprising several drug-binding domains may be synthesized. Alternatively, several of the synthesized drug-binding peptides may be joined together using bifunctional cross-linkers, as described below.” The polymeric material 14, in one embodiment, compriseses more than one drug-binding domain.

By way of yet further illustration, and referring to U.S. Pat. No. 5,420,105 (the entire disclosure of which is hereby incorporated by reference into this specification), the polymeric material 14 may form a conjugate with a ligand. Thus, and referring to claim 1 of such patent, such conjugate may be “A ligand or an anti-ligand/polymeric carrier/drug conjugate comprising a ligand consisting of biotin or an anti-ligand selected from the group consisting of avidin and streptavidin, which ligand or anti-ligand is covalently bound to a polymeric carrier that comprises at least one drug-binding domain derived from a drug-binding protein, and at least one drug non-covalently bound to the polymeric carrier, wherein the polymeric carrier does not comprise an entire drug-binding protein, but is derived from a drug-binding domain of said drug-binding protein which derivative non-covalently binds a drug which is non-covalently bound by an entire naturally occurring drug-binding protein, and wherein the molecular weight of the polymeric carrier is less than about 60,000 daltons, and wherein said drug is selected from the group consisting of an anti-cancer anthracycline antibiotic, cis-platinum, methotrexate, vinblastine, mitoxanthrone ARA-C, 6-mercaptopurine, 6-mercaptoguanosine, mytomycin C and a steroid.” In one embodiment, the polymeric material 14 forms a conjugate with a ligand.

Referring again to FIG. 1, the polymeric material 14 may comprise a reservoir (not shown in FIG. 1, but see U.S. Pat. No. 5,447,724) for the therapeutic agent(s) 18 and/or 20 and/or 22 and/or 24 and/or 26 and/or 28 and/or 30. Such a reservoir may be constructed in accordance with the procedure described in U.S. Pat. No. 5,447,724, which claims “A medical device at least a portion of which comprises: a body insertable into a patient, said body having an exposed surface which is adapted for exposure to tissue of a patient and constructed to release, at a predetermined rate, a therapeutic agent adapted to inhibit adverse physiological reaction of said tissue to the presence of the body of said medical device, said therapeutic agent selected from the group consisting of antithrombogenic agents, antiplatelet agents, prostaglandins, thrombolytic drugs, antiproliferative drugs, antirejection drugs, antimicrobial drugs, growth factors, and anticalcifying agents, at said exposed surface, said body including: an outer polymer metering layer, and an internal polymer layer underlying and supporting said outer polymer metering layer and in intimate contact therewith, said internal polymer layer defining a reservoir for said therapeutic agent, said reservoir formed by a polymer selected from the group consisting of polyurethanes and its copolymers, silicone and its copolymers, ethylene vinylacetate, thermoplastic elastomers, polyvinylchloride, polyolefins, cellulosics, polyamides, polytetrafluoroethylenes, polyesters, polycarbonates, polysulfones, acrylics, and acrylonitrile butadiene styrene copolymers, said outer polymer metering layer having a stable, substantially uniform, predetermined thickness covering the underlying reservoir so that no portion of the reservoir is directly exposed to body fluids and incorporating a distribution of an elutable component which, upon exposure to body fluid, elutes from said outer polymer metering layer to form a predetermined porous network capable of exposing said therapeutic agent in said reservoir in said internal polymer layer to said body fluid, said elutable component is selected from the group consisting of polyethylene oxide, polyethylene glycol, polyethylene oxide/polypropylene oxide copolymers, polyhydroxyethylmethacrylate, polyvinylpyrollidone, polyacrylamide and its copolymers, liposomes, albumin, dextran, proteins, peptides, polysaccharides, polylactides, polygalactides, polyanhydrides, polyorthoesters and their copolymers, and soluble cellulosics, said reservoir defined by said internal polymer layer incorporating said therapeutic agent in a manner that permits substantially free outward release of said therapeutic agent from said reservoir into said porous network of said outer polymer metering layer as said elutable component elutes from said polymer metering layer, said predetermined thickness and the concentration and particle size of said elutable component being selected to enable said outer polymer metering layer to meter the rate of outward migration of the therapeutic agent from said internal reservoir layer through said outer polymer metering layer, said outer polymer metering layer and said internal polymer layer, in combination, enabling prolonged controlled release, at said predetermined rate, of said therapeutic agent at an effective dosage level from said exposed surface of said body of said medical device to the tissue of said patient to inhibit adverse reaction of the patient to the prolonged presence of said body of said medical device in said patient.” In one embodiment, the polymeric material 14 is comprised of a reservoir.

U.S. Pat. No. 5,447,724 also discloses the preparation of the “reservoir” in e.g., in columns 8 and 9 of the patent, wherein it is disclosed that: “A particular advantage of the time-release polymers of the invention is the manufacture of coated articles, i.e., medical instruments. Referring now to FIG. 3, the article to be coated such as a catheter 50 may be mounted on a mandrel or wire 60 and aligned with the preformed apertures 62 (slightly larger than the catheter diameter) in the teflon bottom piece 63 of a boat 64 that includes a mixture 66 of polymer at ambient temperature, e.g., 25° C. To form the reservoir portion, the mixture may include, for example, nine parts solvent, e.g. tetrahydrofuran (THF), and one part Pellthane® polyurethane polymer which includes the desired proportion of ground sodium heparin particles. The boat may be moved in a downward fashion as indicated by arrow 67 to produce a coating 68 on the exterior of catheter 50. After a short (e.g., 15 minutes) drying period, additional coats may be added as desired. After coating, the catheter 50 is allowed to air dry at ambient temperature for about two hours to allow complete solvent evaporation and/or polymerization to form the reservoir portion. For formation of the surface-layer the boat 64 is cleaned of the reservoir portion mixture and filled with a mixture including a solvent, e.g. THF (9 parts) and Pellthane® (1 part) having the desired amount of elutable component. The boat is moved over the catheter and dried, as discussed above to form the surface-layer. Subsequent coats may also be formed. An advantage of the dipping method and apparatus described with regard to FIG. 3 is that highly uniform coating thickness may be achieved since each portion of the substrate is successively in contact with the mixture for the same period of time and further, no deformation of the substrate occurs. Generally, for faster rates of movement of the boat 64, thicker layers are formed since the polymer gels along the catheter surfaces upon evaporation of the solvent, rather than collects in the boat as happens with slower boat motion. For thin layers, e.g., on the order of a few mils, using a fairly volatile solvent such as THF, the dipping speed is generally between 26 to 28 cm/min for the reservoir portion and around 21 cm/min for the outer layer for catheters in the range of 7 to 10 F. The thickness of the coatings may be calculated by subtracting the weight of the coated catheter from the weight of the uncoated catheter, dividing by the calcuated surface area of the uncoated substrate and dividing by the known density of the coating. The solvent may be any solvent that solubilizes the polymer and preferably is a more volatile solvent that evaporates rapidly at ambient temperature or with mild heating. The solvent evaporation rate and boat speed are selected to avoid substantial solubilizing of the catheter substrate or degradation of a prior applied coating so that boundaries between layers are formed.”

By way of yet further illustration, and referring to U.S. Pat. No. 5,464,650 (the entire disclosure of which is hereby incorporated by reference into this specification), the polymeric material 14 may be one or ore of the polymeric materials discussed at columns 4 and 5 of such patent. Referring to such columns 4 and 5, it is disclosed that: “The polymer chosen must be a polymer that is biocompatible and minimizes irritation to the vessel wall when the stent is implanted. The polymer may be either a biostable or a bioabsorbable polymer depending on the desired rate of release or the desired degree of polymer stability, but a bioabsorbable polymer is probably more desirable since, unlike a biostable polymer, it will not be present long after implantation to cause any adverse, chronic local response. Bioabsorbable polymers that could be used include poly(L-lactic acid), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid. Also, biostable polymers with a relatively low chronic tissue response such as polyurethanes, silicones, and polyesters could be used and other polymers could also be used if they can be dissolved and cured or polymerized on the stent such as polyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose. The ratio of therapeutic substance to polymer in the solution will depend on the efficacy of the polymer in securing the therapeutic substance onto the stent and the rate at which the coating is to release the therapeutic substance to the tissue of the blood vessel. More polymer may be needed if it has relatively poor efficacy in retaining the therapeutic substance on the stent and more polymer may be needed in order to provide an elution matrix that limits the elution of a very soluble therapeutic substance. A wide ratio of therapeutic substance to polymer could therefore be appropriate and could range from about 10:1 to about 1:100.”

Referring again to FIG. 1, the therapeutic agent(s) 18 and/or 20 and/or 22 and/or 24 and/or 26 and/or 28 and/or 30 may, e.g., be any one or more of the therapeutic agents disclosed in column 5 of U.S. Pat. No. 5,464,650. Thus, and referring to such column 5, “The therapeutic substance used in the present invention could be virtually any therapeutic substance which possesses desirable therapeutic characteristics for application to a blood vessel. This can include both solid substances and liquid substances. For example, glucocorticoids (e.g. dexamethasone, betamethasone), heparin, hirudin, tocopherol, angiopeptin, aspirin, ACE inhibitors, growth factors, oligonucleotides, and, more generally, antiplatelet agents, anticoagulant agents, antimitotic agents, antioxidants, antimetabolite agents, and anti-inflammatory agents could be used. Antiplatelet agents can include drugs such as aspirin and dipyridamole. Aspirin is classified as an analgesic, antipyretic, anti-inflammatory and antiplatelet drug. Dypridimole is a drug similar to aspirin in that it has anti-platelet characteristics. Dypridimole is also classified as a coronary vasodilator. Anticoagulant agents can include drugs such as heparin, coumadin, protamine, hirudin and tick anticoagulant protein. Antimitotic agents and antimetabolite agents can include drugs such as methotrexate, azathioprine, vincristine, vinblastine, fluorouracil, adriamycin and mutamycin.”

By way of yet further illustration, and referring to U.S. Pat. No. 5,470,307 (the entire disclosure of which is hereby incorporated by reference into this specification), the polymeric material 14 may a synthetic or natural polymer, such as polyamide, polyester, polyolefin (polypropylene or polyethylene), polyurethane, latex, acrylamide, methacrylate, polyvinylchloride, polysuflone, and the like; see, e.g., column 11 of the patent.

Referring again to FIG. 1A, the polymeric material 14 may be bound to the therapeutic agent(s) 18 and/or 20 and/or 22 and/or 24 and/or 26 and/or 28 by a linker, such as a photosensitive linker 37; although only one such photosensitive linker 37 is depicted in FIG. 1A, it will be apparent to those skilled in the art that many such photosensitive linkers are preferably bound to polymeric material 14.

In another embodiment, depicted in FIG. 1A, the photosensitive linker 37 is bound to layer 16 comprised of nanomagnetic material. In yet another embodiment, the photosensitive linker 37 is bound to the surface of container 12. Combinations of these bound linkers, and/or different therapeutic agents, may be used.

This process of preparing and binding these photosensitive linkers is described in columns 8-9 of U.S. Pat. No. 5,470,307, wherein it is disclosed that: “The process of fabricating a catheter 10 having a desired therapeutic agent 20 connected thereto and then controllably and selectively releasing that therapeutic agent 20 at a remote site within a patient may be summarized in five steps. 1. Formation of Substrate. The substrate layer 16 is formed on or applied to the surface 14 of the catheter body 12, and subsequently or simultaneously prepared for coupling to the linker layer 18. This is accomplished by modifying the substrate layer 16 to expose or add groups such as carboxyls, amines, hydroxyls, or sulfhydryls. In some cases, this may be followed by customizing the substrate layer 16 with an extender 22 that will change the functionality, for example by adding a maleimide group that will accept a Michael's addition of a sulfhydryl at one end of a bifunctional photolytic linker 18. The extent of this derivitization is measured by adding group-specific probes (such as 1 pyrenyl diazomethane for carboxyls, 1 pyrene butyl hydrazine for amines, or Edman's reagent for sulfhydryls Molecular Probes, Inc. of Eugene, Oreg. or Pierce Chemical of Rockford, Ill.) or other fluorescent dyes that may be measured optically or by flow cytometry. The substrate layer 16 can be built up to increase its capacity by several methods, examples of which are discussed below.”

As is also dislosed in U.S. Pat. No. 5,470,307, “2. Selection of Photolytic Release Mechanism. A heterobifunctional photolytic linker 18 suitable for the selected therapeutic agent 20 and designed to couple readily to the functionality of the substrate layer 16 is prepared, and may be connected to the substrate layer 16. Alternately, the photolinker 18 may first be bonded to the therapeutic agent 20, with the combined complex of the therapeutic agent 20 and photolytic linker 18 together being connected to the substrate layer 16. 3. Selection of the Therapeutic Agent. Selection of the appropriate therapeutic agent 20 for a particular clinical application will depend upon the prevailing medical practice. One representative example described below for current use in PTCA and PTA procedures involves the amine terminal end of a twelve amino acid peptide analogue of hirudin being coupled to a chloro carbonyl group on the photolytic linker 18. Another representative example is provided below where the therapeutic agent 20 is a nucleotide such as an antisense oligodeoxynucleotide where a terminal phosphate is bonded by means of a diazoethane located on the photolytic linker 18. A third representative example involves the platelet inhibitor dipyridamole (persantin) that is attached through an alkyl hydroxyl by means of a diazo ethane on the photolytic linker 18. 4. Fabrication of the Linker-Agent Complex and Attachment to the Substrate. The photolytic linker 18 or the photolytic linker 18 with the therapeutic agent 20 attached are connected to the substrate layer 16 to complete the catheter 10. A representative example is a photolytic linker 18 having a sulfhydryl disposed on the non-photolytic end for attachment to the substrate layer 16, in which case the coupling will occur readily in a neutral buffer solution to a maleimide-modified substrate layer 16 on the catheter 10. Once the therapeutic agent 20 has been attached to the catheter 10, it is necessary that the catheter 10 be handled in a manner that prevents damage to the substrate layer 16, photolytic linker layer 18, and therapeutic agent 20, which may include subsequent sterilization, protection from ambient light, heat, moisture, and other environmental conditions that would adversely affect the operation or integrity of the drug-delivery catheter system 10 when used to accomplish a specific medical procedure on a patient.”

In the process of U.S. Pat. No. 5,470,307, the linker is preferably bound to the polymeric material through a modified functional group. The preparation of such modified functional groups is discussed at columns 10-13 of such patent, wherein it is disclosed that: “Most polymers including those discussed herein can be made of materials which have modifiable functional groups or can be treated to expose such groups. Polyamide (nylon) can be modified by acid treatment to produce exposed amines and carboxyls. Polyethylene terephthalate (PET, Dacron®) is a polyester and can be chemically treated to expose hydroxyls and carboxyls. Polystyrene has an exposed phenyl group that can be derivitized. Polyethylene and polypropylene (collectively referred to as polyolefins) have simple carbon backbones which can be derivitized by treatment with chromic and nitric acids to produce carboxyl functionality, photocoupling with suitably modified benzophenones, or by plasma grafting of selected monomers to produce the desired chemical functionality. For example, grafting of acrylic acid will produce a surface with a high concentration of carboxyl groups, whereas thiophene or 1,6 diaminocyclohexane will produce a surface containing sulfhydryls or amines, respectively. The surface functionality can be modified after grafting of a monomer by addition of other functional groups. For example, a carboxyl surface can be changed to an amine by coupling 1,6 diamino hexane, or to a sulfhydryl surface by coupling mercapto ethyl amine.”

As is also dislosed in U.S. Pat. No. 5,470,307, “Acrylic acid can be polymerized onto latex, polypropylene, polysulfone, and polyethylene terephthalate (PET) surfaces by plasma treatment. When measured by toluidine blue dye binding, these surfaces show intense modification. On polypropylene microporous surfaces modified by acrylic acid, as much as 50 nanomoles of dye binding per cm2 of external surface area can be found to represent carboxylated surface area. Protein can be linked to such surfaces using carbonyl diimidazole (CDI) in tetrahydrofuran as a coupling system, with a resultant concentration of one nanomole or more per cm2 of external surface. For a 50,000 Dalton protein, this corresponds to 50 μg per cm2, which is far above the concentration expected with simple plating on the surface. Such concentrations of a therapeutic agent 20 on the angioplasty (PTCA) balloon of a catheter 10, when released, would produce a high concentration of that therapeutic agent 20 at the site of an expanded coronary artery. However, plasma-modified surfaces are difficult to control and leave other oxygenated carbons that may cause undesired secondary reactions”

As is also dislosed in U.S. Pat. No. 5,470,307, “In the case of balloon dilation catheters 10, creating a catheter body 12 capable of supporting a substrate layer 16 with enhanced surface area can be done by several means known to the art including altering conditions during balloon spinning, doping with appropriate monomers, applying secondary coatings such as polyethylene oxide hydrogel, branched polylysines, or one of the various Starburst.TM. dendrimers offered by the Aldrich Chemical Company of Milwaukee, Wis.”

As is also dislosed in U.S. Pat. No. 5,470,307, “The most likely materials for the substrate layer 16 in the case of a dilation balloon catheter 10 or similar apparatus are shown in FIGS. 1a-1g, including synthetic or natural polymers such as polyamide, polyester, polyolefin (polypropylene or polyethylene), polyurethane, and latex. For solid support catheter bodies 12, usable plastics might include acrylamides, methacrylates, urethanes, polyvinylchloride, polysulfone, or other materials such as glass or quartz, which are all for the most part derivitizable.” In one embodiment, depicted in FIG. 1A, the photosensitive linker is bonded to a plastic container 12.

As is also dislosed in U.S. Pat. No. 5,470,307, “Referring to the polymers shown in FIGS. 1a-1g, polyamide (nylon) is treated with 3-5M hydrochloric acid to expose amines and carboxyl groups using conventional procedures developed for enzyme coupling to nylon tubing. A further description of this process may be obtained from Inman, D. J. and Hornby, W. E., The Iramobilization of Enzymes on Nylon Structures and their Use in Automated Analysis, Biochem. J. 129:255-262 (1972) and Daka, N. J. and Laidler, Flow kinetics of lactate dehydrogenase chemically attached to nylon tubing, K. J., Can. J. Biochem. 56:774-779 (1978). This process will release primary amines and carboxyls. The primary amine group can be used directly, or succinimidyl 4 (p-maleimidophenyl) butyrate (SMBP) can be coupled to the amine function leaving free the maleimide to couple with a sulfhydryl on several of the photolytic linkers 18 described below and acting as an extender 22. If needed, the carboxyl released can also be converted to an amine by first protecting the amines with BOC groups and then coupling a diamine to the carboxyl by means of carbonyl diimidazole (CDI).” The polymeric material 14, and/or the container 12, may comprise or consist essentially of nylon.

As is also dislosed in U.S. Pat. No. 5,470,307, “Polyester (Dacron®) can be functionalized using 0.01N NaOH in 10% ethanol to release hydroxyl and carboxyl groups in the manner described by Blassberger, D. et al, Chemically Modified Polyesters as Supports for Enzyme Iramobilization: lsocyanide, Acylhydrazine, and Aminoaryl derivatives of Poly(ethylene Terephthalate), Biotechnol. and Bioeng. 20:309-315 (1978). A diamine is added directly to the etched surface using CDI and then reacted with SMBP to yield the same maleimide reacting group to accept the photolytic linker 18.” The polymeric material 14, and/or the container 12, may comprise or consist essentially of polyester.

As is also dislosed in U.S. Pat. No. 5,470,307, “Polystyrene can be modified many ways, however perhaps the most useful process is chloromethylation, as originally described by Merrifield, R. B., Solid Phase Synthesis. I. The Synthesis of a Tetrapeptide, J. Am. Chem Soc. 85:2149-2154 (1963), and later discussed by Atherton, E. and Sheppard, R. C., Solid Phase Peptide Synthesis: A Practical Approach, pp. 13-23, (IRL Press 1989). The chlorine can be modified to an amine by reaction with anhydrous ammonia.” The polymeric material 14, and/or the container 12, may be comprised of or consist essentially of polystyrene.

As is also dislosed in U.S. Pat. No. 5,470,307, “Polyolefins (polypropylene or polyethylene) require different approaches because they contain primarily a carbon backbone offering no native functional groups. One suitable approach is to add carboxyls to the surface by oxidizing with chromic acid followed by nitric acid as described by Ngo, T. T. et al., Kinetics of acetylcholinesterase immobilized on polyethylene tubing, Can. J. Biochem. 57:1200-1203 (1979). These carboxyls are then converted to amines by reacting successively with thionyl chloride and ethylene diamine. The surface is then reacted with SMBP to produce a maleimide that will react with the sulfhydryl on the photolytic linker 18.” The polymeric material 14, and/or the container 12, may be comprised of or consist essentially of polyolefin material.

As is also dislosed in U.S. Pat. No. 5,470,307, “A more direct method is to react the polyolefin surfaces with benzophenone 4-maleimide as described by Odom, O. W. et al, Relaxation Time, Interthiol Distance, and Mechanism of Action of Ribosomal Protein S1, Arch. Biochem Biophys. 230:178-193 (1984), to produce the required group for the sulfhydryl addition to the photolytic linker 18. The benzophenone then links to the polyolefin through exposure to ultraviolet (uv) light. Other methods to derivitize the polyolefin surface include the use of radio frequency glow discharge (RFGD)—also known as plasma discharge—in several different manners to produce an in-depth coating to provide functional groups as well as increasing the effective surface area. Polyethylene oxide (PEO) can be crosslinked to the surface, or polyethylene glycol (PEG) can also be used and the mesh varied by the size of the PEO or PEG. This is discussed more fully by Sheu, M. S., et al., A glow discharge treatment to immobilize poly(ethylene oxide)/poly(propylene oxide) surfactants for wettable and non-fouling biomaterials, J. Adhes. Sci. Tech., 6:995-1009 (1992) and Yasuda, H., Plasma Polymerization, (Academic Press, Inc. 1985). Exposed hydroxyls can be activated by tresylation, also known as trifluoroethyl sulfonyl chloride activation, in the manner described by Nielson, K. and Mosbach, K., Tresyl Chloride-Activated Supports for Enzyme Immobilization (and related articles), Meth. Enzym., 135:65-170 (1987). The function can be converted to amines by addition of ethylene diamine or other aliphatic diamines, and then the usual addition of SMBP will give the required maleimide. Another suitable method is to use RFGD to polymerize acrylic acid or other monomers on the surface of the polyolefin. This surface consisting of carboxyls and other carbonyls is derivitizable with CDI and a diamine to give an amine surface which then can react with SMBP.”

Referring again to the process described in U.S. Pat. No. 5,470,307, photolytic linkers can be conjugated to the functional groups on the substrate layers 16 to form linker-agent complexes. As is disclosed in columns 13-14 of such patent, “Once a particular functionality for the substrate layer 16 has been determined, the appropriate strategy for coupling the photolytic linker 18 can be selected and employed. Several such strategies are set out in the examples which follow. As with selecting a method to expose a functional group on the surface 14 of the substrate layer 16, it is understood that selection of the appropriate strategy for coupling the photolytic linker 18 will depend upon various considerations including the chemical functionality of the substrate layer 16, the particular therapeutic agent 20 to be used, the chemical and physical factors affecting the rate and equilibrium of the particular photolytic release mechanism, the need to minimize any deleterious side-effects that might result (such as the production of antagonistic or harmful chemical biproducts, secondary chemical reactions with adjunct medical instruments including other portions of the catheter 10, unclean leaving groups or other impurities), and the solubility of the material used to fabricate the catheter body 12 or substrate layer 16 in various solvents. More limited strategies are available for the coupling of a 2-nitrophenyl photolytic linker 18. If the active site is 1-ethyl hydrazine used in most caging applications, then the complementary functionality on the therapeutic agent 20 will be a carboxyl, hydroxyl, or phosphate available on many pharmaceutical drugs. If a bromomethyl group is built into the photolytic linker 18, it can accept either a carboxyl or one of many other functional groups, or be converted to an amine which can then be further derivitized. In such a case, the leaving group might not be clean and care must be taken when adopting this strategy for a particular therapeutic agent 20. Other strategies include building in an oxycarbonyl in the 1-ethyl position, which can form an urethane with an amine in the therapeutic agent 20. In this case, the photolytic process evolves CO2.”

Referring again to U.S. Pat. No. 5,470,307, after the photolytic linker construct has been prepared, it may be contacted with a coherent laser light source 39 (see FIG. 1A) to release the therapeutic agent. Thus, as is disclosed in column 9 of U.S. Pat. No. 5,470,307, “use of a coherent laser light source 26 will be preferable in many applications because the use of one or more discrete wavelengths of light energy that can be tuned or adjusted to the particular photolytic reaction occurring in the photolytic linker 18 will necessitate only the minimum power (wattage) level necessary to accomplish a desired release of the therapeutic agent 20. As discussed above, coherent or laser light sources 26 are currently used in a variety of medical procedures including diagnostic and interventional treatment, and the wide availability of laser sources 26 and the potential for redundant use of the same laser source 26 in photolytic release of the therapeutic agent 20 as well as related procedures provides a significant advantage. In addition, multiple releases of different therapeutic agents 20 or multiple-step reactions can be accomplished using coherent light of different wavelengths, intermediate linkages to dye filters may be utilized to screen out or block transmission of light energy at unused or antagonistic wavelengths (particularly cytotoxic or cytogenic wavelengths), and secondary emitters may be utilized to optimize the light energy at the principle wavelength of the laser source 26. In other applications, it may be suitable to use a light source 26 such as a flash lamp operatively connected to the portion of the body 12 of the catheter 10 on which the substrate 16, photolytic linker layer 18, and therapeutic agent 20 are disposed. One example would be a mercury flash lamp capable of producing long-wave ultra-violet (uv) radiation within or across the 300-400 nanometer wavelength spectrum. When using either a coherent laser light source 26 or an alternate source 26 such as a flash lamp, it is generally preferred that the light energy be transmitted through at least a portion of the body 12 of the catheter 10 such that the light energy traverses a path through the substrate layer 16 to the photolytic linker layer 18 in order to maximize the proportion of light energy transmitted to the photolytic linker layer 18 and provide the greatest uniformity and reproducibility in the amount of light energy (photons) reaching the photolytic linker layer 18 from a specified direction and nature. Optimal uniformity and reproducibility in exposure of the photolyric linker layer 18 permits advanced techniques such as variable release of the therapeutic agent 20 dependent upon the controlled quantity of light energy incident on the substrate layer 16 and photolytic linker layer 18.”

As is also dislosed in U.S. Pat. No. 5,470,307, “The art pertaining to the transmission of light energy through fiber optic conduits 28 or other suitable transmission or production means to the remote biophysical site is extensively developed. For a fiber optic device, the fiber optic conduit 28 material must be selected to accommodate the wavelengths needed to achieve release of the therapeutic agent 20 which will for almost all applications be within the range of 280-400 nanometers. Suitable fiber optic materials, connections, and light energy sources 26 may be selected from those currently available and utilized within the biomedical field. While fiber optic conduit 28 materials may be selected to optimize transmission of light energy at certain selected wavelengths for desired application, the construction of a catheter 10 including fiber optic conduit 28 materials capable of adequate transmission throughout the range of the range of 280-400 nanometers is preferred, since this catheter 10 would be usable with the full compliment of photolytic release mechanisms and therapeutic agents 10. Fabrication of the catheter 10 will therefore depend more upon considerations involving the biomedical application or procedure by which the catheter 10 will be introduced or implanted in the patient, and any adjunct capabilities which the catheter 10 must possess.”

By way of yet further illustration, and referring to U.S. Pat. No. 5,599,352 (the entire disclosure of which is hereby incorporated by reference into this specification), the polymeric material 14 can comprise fibrin. As is disclosed in column 4 of such patent, “The present invention provides a stent comprising fibrin. The term “fibrin” herein means the naturally occurring polymer of fibrinogen that arises during blood coagulation. Blood coagulation generally requires the participation of several plasma protein coagulation factors: factors XII, XI, IX, X, VIII, VII, V, XIII, prothrombin, and fibrinogen, in addition to tissue factor (factor III), kallikrein, high molecular weight kininogen, Ca+2, and phospholipid. The final event is the formation of an insoluble, cross-linked polymer, fibrin, generated by the action of thrombin on fibrinogen. Fibrinogen has three pairs of polypeptide chains (ALPHA 2—BETA 2—GAMMA 2) covalently linked by disulfide bonds with a total molecular weight of about 340,000. Fibrinogen is converted to fibrin through proteolysis by thrombin. An activation peptide, fibrinopeptide A (human) is cleaved from the amino-terminus of each ALPHA chain; fibrinopeptide B (human) from the amino-terminus of each BETA chain. The resulting monomer spontaneously polymerizes to a fibrin gel. Further stabilization of the fibrin polymer to an insoluble, mechanically strong form, requires cross-linking by factor XIII. Factor XIII is converted to XIIIa by thrombin in the presence of Ca+2. XIIIa cross-links the GAMMA chains of fibrin by transglutaminase activity, forming EPSILON-(GAMMA-glutamyl) lysine cross-links. The ALPHA chains of fibrin also may be secondarily cross-linked by transamidation.”

As is also dislosed in U.S. Pat. No. 5,599,352, “Since fibrin blood clots are naturally subject to fibrinolysis as part of the body's repair mechanism, implanted fibrin can be rapidly biodegraded. Plasminogen is a circulating plasma protein that is adsorbed onto the surface of the fibrin polymer. The adsorbed plasminogen is converted to plasmin by plasminogen activator released from the vascular endothelium. The plasmin will then break down the fibrin into a collection of soluble peptide fragments.”

As is also dislosed in U.S. Pat. No. 5,599,352, “Methods for making fibrin and forming it into implantable devices are well known as set forth in the following patents and published applications which are hereby incorporated by reference. In U.S. Pat. No. 4,548,736 issued to Muller et al., fibrin is clotted by contacting fibrinogen with a fibrinogen-coagulating protein such as thrombin, reptilase or ancrod. Preferably, the fibrin in the fibrin-containing stent of the present invention has Factor XIII and calcium present during clotting, as described in U.S. Pat. No. 3,523,807 issued to Gerendas, or as described in published European Patent Application 0366564, in order to improve the mechanical properties and biostability of the implanted device. Also preferably, the fibrinogen and thrombin used to make fibrin in the present invention are from the same animal or human species as that in which the stent of the present invention will be implanted in order to avoid cross-species immune reactions. The resulting fibrin can also be subjected to heat treatment at about 150° C. for 2 hours in order to reduce or eliminate antigenicity. In the Muller patent, the fibrin product is in the form of a fine fibrin film produced by casting the combined fibrinogen and thrombin in a film and then removing moisture from the film osmotically through a moisture permeable membrane. In the European Patent Application 0366564, a substrate (preferably having high porosity or high affinity for either thrombin or fibrinogen) is contacted with a fibrinogen solution and with a thrombin solution. The result is a fibrin layer formed by polymerization of fibrinogen on the surface of the device. Multiple layers of fibrin applied by this method could provide a fibrin layer of any desired thickness. Or, as in the Gerendas patent, the fibrin can first be clotted and then ground into a powder which is mixed with water and stamped into a desired shape in a heated mold. Increased stability can also be achieved in the shaped fibrin by contacting the fibrin with a fixing agent such as glutaraldehyde or formaldehyde. These and other methods known by those skilled in the art for making and forming fibrin may be used in the present invention.”

As is also dislosed in U.S. Pat. No. 5,599,352, “Preferably, the fibrinogen used to make the fibrin is a bacteria-free and virus-free fibrinogen such as that described in U.S. Pat. No. 4,540,573 to Neurath et al which is hereby incorporated by reference. The fibrinogen is used in solution with a concentration between about 10 and 50 mg/ml and with a pH of about 5.8-9.0 and with an ionic strength of about 0.05 to 0.45. The fibrinogen solution also typically contains proteins and enzymes such as albumin, fibronectin (0-300 μg per ml fibrinogen), Factor XIII (0-20 μg per ml fibrinogen), plasminogen (0-210 μg per ml fibrinogen), antiplasmin (0-61 μg per ml fibrinogen) and Antithrombin m (0-150 μg per ml fibrinogen). The thrombin solution added to make the fibrin is typically at a concentration of 1 to 120 NIH units/ml with a preferred concentration of calcium ions between about 0.02 and 0.2M.”

As is also dislosed in U.S. Pat. No. 5,599,352, “Polymeric materials can also be intermixed in a blend or co-polymer with the fibrin to produce a material with the desired properties of fibrin with improved structural strength. For example, the polyurethane material described in the article by Soldani et at., “Bioartificial Polymeric Materials Obtained from Blends of Synthetic Polymers with Fibrin and Collagen” International Journal of Artificial Organs, Vol. 14, No. 5, 1991, which is incorporated herein by reference, could be sprayed onto a suitable stent structure. Suitable polymers could also be biodegradable polymers such as polyphosphate ester, polyhydroxybutyrate valerate, polyhydroxybutyrate-co-hydroxyvalerate and the like . . . ” The polymeric material 14 may be, e.g., a blend of fibrin and another polymeric material.

As is also dislosed in U.S. Pat. No. 5,599,352, “The shape for the fibrin can be provided by molding processes. For example, the mixture can be formed into a stent having essentially the same shape as the stent shown in U.S. Pat. No. 4,886,062 issued to Wiktor. Unlike the method for making the stent disclosed in Wiktor which is wound from a wire, the stent made with fibrin can be directly molded into the desired open-ended tubular shape.”

As is also dislosed in U.S. Pat. No. 5,599,352, “In U.S. Pat. No. 4,548,736 issued to Muller et al., a dense fibrin composition is disclosed which can be a bioabsorbable matrix for delivery of drugs to a patient. Such a fibrin composition can also be used in the present invention by incorporating a drug or other therapeutic substance useful in diagnosis or treatment of body lumens to the fibrin provided on the stent. The drug, fibrin and stent can then be delivered to the portion of the body lumen to be treated where the drug may elute to affect the course of restenosis in surrounding luminal tissue. Examples of drugs that are thought to be useful in the treatment of restenosis are disclosed in published international patent application WO 91/12779 “Intraluminal Drug Eluting Prosthesis” which is incorporated herein by reference. Therefore, useful drugs for treatment of restenosis and drugs that can be incorporated in the fibrin and used in the present invention can include drugs such as anticoagulant drugs, antiplatelet drugs, antimetabolite drugs, anti-inflammatory drugs and antimitotic drugs. Further, other vasoreactive agents such as nitric oxide releasing agents could also be used. Such therapeutic substances can also be microencapsulated prior to their inclusion in the fibrin. The micro-capsules then control the rate at which the therapeutic substance is provided to the blood stream or the body lumen. This avoids the necessity for dehydrating the fibrin as set forth in Muller et al., since a dense fibrin structure would not be required to contain the therapeutic substance and limit the rate of delivery from the fibrin. For example, a suitable fibrin matrix for drug delivery can be made by adjusting the pH of the fibrinogen to below about pH 6.7 in a saline solution to prevent precipitation (e.g., NACl, CaCl, etc.), adding the microcapsules, treating the fibrinogen with thrombin and mechanically compressing the resulting fibrin into a thin film. The microcapsules which are suitable for use in this invention are well known. For example, the disclosures of U.S. Pat. Nos. 4,897,268, 4,675,189; 4,542,025; 4,530,840; 4,389,330; 4,622,244; 4,464,317; and 4,943,449 could be used and are incorporated herein by reference. Alternatively, in a method similar to that disclosed in U.S. Pat. No. 4,548,736 issued to Muller et al., a dense fibrin composition suitable for drug delivery can be made without the use of microcapsules by adding the drug directly to the fibrin followed by compression of the fibrin into a sufficiently dense matrix that a desired elution rate for the drug is achieved. In yet another method for incorporating drugs which allows the drug to elute at a controlled rate, a solution which includes a solvent, a polymer dissolved in the solvent and a therapeutic drug dispersed in the solvent is applied to the structural elements of the stent and then the solvent is evaporated. Fibrin can then be added over the coated structural elements in an adherent layer. The inclusion of a polymer in intimate contact with a drug on the underlying stent structure allows the drug to be retained on the stent in a resilient matrix during expansion of the stent and also slows the administration of drug following implantation. The method can be applied whether the stent has a metallic or polymeric surface. The method is also an extremely simple method since it can be applied by simply immersing the stent into the solution or by spraying the solution onto the stent. The amount of drug to be included on the stent can be readily controlled by applying multiple thin coats of the solution while allowing it to dry between coats. The overall coating should be thin enough so that it will not significantly increase the profile of the stent for intravascular delivery by catheter. It is therefore preferably less than about 0.002 inch thick and most preferably less than 0.001 inch thick. The adhesion of the coating and the rate at which the drug is delivered can be controlled by the selection of an appropriate bioabsorbable or biostable polymer and by the ratio of drug to polymer in the solution. By this method, drugs such as glucocorticoids (e.g. dexamethasone, betamethasone), heparin, hirudin, tocopherol, angiopeptin, aspirin, ACE inhibitors, growth factors, oligonucleotides, and, more generally, antiplatelet agents, anticoagulant agents, antimitotic agents, antioxidants, antimetabolite agents, and anti-inflammatory agents can be applied to a stent, retained on a stent during expansion of the stent and elute the drug at a controlled rate. The release rate can be further controlled by varying the ratio of drug to polymer in the multiple layers. For example, a higher drug-to-polymer ratio in the outer layers than in the inner layers would result in a higher early dose which would decrease over time. Examples of some suitable combinations of polymer, solvent and therapeutic substance are set forth in Table 1 below . . . .”

At column 7 of U.S. Pat. No. 5,599,352, some polymers that can be mixed with the fibrin are discussed. It is disclosed that: “The polymer used can be a bioabsorbable or biostable polymer. Suitable bioabsorbable polymers include poly(L-lactic acid), poly(lactide-co-glycolide) and poly(hydroxybutyrate-co-valerate). Suitable biostable polymers include silicones, polyurethanes, polyesters, vinyl homopolymers and copolymers, acrylate homopolymers and copolymers, polyethers and cellulosics. A typical ratio of drug to dissolved polymer in the solution can vary widely (e.g. in the range of about 10:1 to 1:100). The fibrin is applied by molding a polymerization mixture of fibrinogen and thrombin onto the composite as described herein.” The polymeric material 14 may be, e.g., a blend of fibrin and a bioabsorbable and/or biostable polymer.

By way of yet further illustration, and referring to U.S. Pat. No. 5,605,696, the polymeric material 14 can be a multi-layered polymeric material, and/or a porous polymeric material. Thus, e.g., and as is disclosed in claim 25 of such patent, “A polymeric material containing a therapeutic drug for application to an intravascular stent for carrying and delivering said therapeutic drug within a blood vessel in which said intravascular stent is placed, comprising: a polymeric material having a thermal processing temperature no greater than about 100° C.; particles of a therapeutic drug incorporated in said polymeric material; and a porosigen uniformly dispersed in said polymeric material, said porosigen being selected from the group consisting of sodium chloride, lactose, sodium heparin, polyethylene glycol, copolymers of polyethylene oxide and polypropylene oxide, and mixtures thereof.” The “porsigen” is described at columns 4 and 5 of the patent, wherein it is disclosed that: “porosigen can also be incorporated in the drug loaded polymer by adding the porosigen to the polymer along with the therapeutic drug to form a porous, drug loaded polymeric membrane. A porosigen is defined herein for purposes of this application as any moiety, such as microgranules of sodium chloride, lactose, or sodium heparin, for example, which will dissolve or otherwise be degraded when immersed in body fluids to leave behind a porous network in the polymeric material. The pores left by such porosigens can typically be a large as 10 microns. The pores formed by porosigens such as polyethylene glycol (PEG), polyethylene oxide/polypropylene oxide (PEO/PPO) copolymers, for example, can also be smaller than one micron, although other similar materials which form phase separations from the continuous drug loaded polymeric matrix and can later be leached out by body fluids can also be suitable for forming pores smaller than one micron. While it is currently preferred to apply the polymeric material to the structure of a stent while the therapeutic drug and porosigen material are contained within the polymeric material, to allow the porosigen to be dissolved or degraded by body fluids when the stent is placed in a blood vessel, alternatively the porosigen can be dissolved and removed from the polymeric material to form pores in the polymeric material prior to placement of the polymeric material combined with the stent within a blood vessel. If desired, a rate-controlling membrane can also be applied over the drug loaded polymer, to limit the release rate of the therapeutic drug. Such a rate-controlling membrane can be useful for delivery of water soluble substances where a nonporous polymer film would completely prevent diffusion of the drug. The rate-controlling membrane can be added by applying a coating from a solution, or a lamination, as described previously. The rate-controlling membrane applied over the polymeric material can be formed to include a uniform dispersion of a porosigen in the rate-controlling membrane, and the porosigen in the rate-controlling membrane can be dissolved to leave pores in the rate-controlling membrane typically as large as 10 microns, or as small as 1 micron, for example, although the pores can also be smaller than 1 micron. The porosigen in the rate-controlling membrane can be, for example, sodium chloride, lactose, sodium heparin, polyethylene glycol, polyethylene oxide/polypropylene oxide copolymers, and mixtures thereof.” The polymeric material 14 may comprise a multiplicity of layers of polymeric material.

Referring again to FIG. 1, one may use any of the therapeutic agents disclosed at columns 3 and 4 of U.S. Pat. No. 5,605,696 as agents 18 and/or 20 and/or 22 and/or 24 and/or 26 and/or 28 and/or 30. Thus, and referring to such patent, “The selected therapeutic drug can, for example, be anticoagulant antiplatelet or antithrombin agents such as heparin, D-phe-pro-arg-chloromethylketone (synthetic antithrombin), dipyridamole, hirudin, recombinant hirudin, thrombin inhibitor (available from Biogen), or c7E3 (an antiplatelet drug from Centocore); cytostatic or antiproliferative agents such as angiopeptin (a somatostatin analogue from Ibsen), angiotensin converting enzyme inhibitors such as Captopril (available from Squibb), Cilazapril (available from Hoffman-LaRoche), or Lisinopril (available from Merk); calcium channel blockers (such as Nifedipine), colchicine, fibroblast growth factor (FGF) antagonists, fish oil (omega 3-fatty acid), low molecular weight heparin (available from Wyeth, and Glycomed), histamine antagonists, Lovastatin (an inhibitor of HMG-CoA reductase, a cholesterol lowering drug from Merk), methotrexate, monoclonal antibodies (such as to PDGF receptors), nitroprusside, phosphodiesterase inhibitors, prostacyclin and prostacyclin analogues, prostaglandin inhibitor (available from Glaxo), Seramin (a PDGF antagonist), serotonin blockers, steroids, thioprotease inhibitors, and triazolopyrimidine (a PDGF antagonist). Other therapeutic drugs which may be appropriate include alphainterferon and genetically engineered epithelial cells, for example.”

By way of yet further illustration, and referring to U.S. Pat. No. 5,700,286 (the entire disclosure of which is hereby incorporated by reference into this specification), the polymeric material 14 may be either a thermoplastic or an elastomeric polymer. Thus, and referring to columns 5 and 6 of such patent, “The polymeric material is preferably selected from thermoplastic and elastomeric polymers. In one currently preferred embodiment the polymeric material can be a material available under the trade name “C-Flex” from Concept Polymer Technologies of Largo, Fla. In another currently preferred embodiment, the polymeric material can be ethylene vinyl acetate (EVA); and in yet another currently preferred embodiment, the polymeric material can be a material available under the trade name “BIOSPAN.” Other suitable polymeric materials include latexes, urethanes, polysiloxanes, and modified styrene-ethylene/butylene-styrene block copolymers (SEBS) and their associated families, as well as elastomeric, bioabsorbable, linear aliphatic polyesters. The polymeric material can typically have a thickness in the range of about 0.002 to about 0.020 inches, for example. The polymeric material is preferably bioabsorbable, and is preferably loaded or coated with a therapeutic agent or drug, including, but not limited to, antiplatelets, antithrombins, cytostatic and antiproliferative agents, for example, to reduce or prevent restenosis in the vessel being treated. The therapeutic agent or drug is preferably selected from the group of therapeutic agents or drugs consisting of sodium heparin, low molecular weight heparin, hirudin, argatroban, forskolin, vapiprost, prostacyclin and prostacyclin analogues, dextran, D-phe-pro-arg-chloromethylketone, dipyridamole, glycoprotein IIb/IIIa platelet membrane receptor antibody, recombinant hirudin, thrombin inhibitor, angiopeptin, angiotensin converting enzyme inhibitors, (such as Captopril, available from Squibb; Cilazapril, available for Hoffman-La Roche; or Lisinopril, available from Merck) calcium channel blockers, colchicine, fibroblast growth factor antagonists, fish oil, omega 3-fatty acid, histamine antagonists, HMG-CoA reductase inhibitor, methotrexate, monoclonal antibodies, nitroprusside, phosphodiesterase inhibitors, prostaglandin inhibitor, seramin, serotonin blockers, steroids, thioprotease inhibitors, triazolopyrimidine and other PDGF antagonists, alpha-interferon and genetically engineered epithelial cells, and combinations thereof. While the foregoing therapeutic agents have been used to prevent or treat restenosis and thrombosis, they are provided by way of example and are not meant to be limiting, as other therapeutic drugs may be developed which are equally applicable for use with the present invention.”

By way of yet further illustration, and referring to U.S. Pat. No. 5,900,433 (the entire disclosure of which is hereby incorporated by reference into this specification), the polymeric material 14 may be a biodegradable controlled release polymer comprised of a congener of an endothelium-derived bioactive composition of matter. This congener is discussed in column 7 of the patent, wherein it is disclosed that “We have discovered that administration of a congener of an endothelium-derived bioactive agent, more particularly a nitrovasodilator, representatively the nitric oxide donor agent sodium nitroprusside, to an extravascular treatment site, at a therapeutically effective dosage rate, is effective for abolishing CFR's while reducing or avoiding systemic effects such as supression of platelet function and bleeding. By “extravascular treatment site”, we mean a site proximately adjacent the exterior of the vessel. In accordance with our invention, congeners of an endothelium-derived bioactive agent include prostacyclin, prostaglandin E1, and a nitrovasodilator agent. Nitrovasodilater agents include nitric oxide and nitric oxide donor agents, including L-arginine, sodium nitroprusside and nitroglycycerine. The so administered nitrovasodilators are effective to provide one or more of the therapeutic effects of promotion of vasodilation, inhibition of vessel spasm, inhibition of platelet aggregation, inhibition of vessel thrombosis, and inhibition of platelet growth factor release, at the treatment site, without inducing systemic hypotension or anticoagulation. The treatment site may be any blood vessel. The most acute such blood vessels are coronary blood vessels. The coronary blood vessel may be a natural artery or an artificial artery, such as a vein graft for arterial bypass. The step of administering includes delivering the congener in a controlled manner over a sustained period of time, and comprises intrapericardially or transpericardially extravascularly delivering the congener to the coronary blood vessel. Methods of delivery comprise (i) either intrapericardially or transpericardially infusing the congener through a percutaneously inserted catheter extravascularly to the coronary blood vessel, (ii) iontophoretically delivering the congener transpericardially extravascularly to the coronary blood vessel, and (iii) inserting extravascularly to the coronary blood vessel an implant capable of extended time release of the congener. The last method of delivery includes percutaneously inserting the implant proximately adjacent, onto, or into the pericardial sac surrounding the heart, and in a particular, comprises surgically wrapping the implant around a vein graft used for an arterial bypass. The extravascular implant may be a biodegradable controlled-release polymer comprising the congener.”

By way of yet further illustration, and referring to U.S. Pat. No. 6,004,346 (the entire disclosure of which is hereby incorporated by reference into this specification), the polymeric material 14 may be a bioabsorbable polymer. Thus, and referring to column 7 of such patent, “controlled release, via a bioabsorbable polymer, offers to maintain the drug level within the desired therapeutic range for the duration of the treatment. In the case of stents, the prosthesis materials will maintain vessel support for at least two weeks or until incorporated into the vessel wall even with bioabsorbable, biodegradable polymer constructions.”

As is also dislosed in U.S. Pat. No. 6,004,346, “Several polymeric compounds that are known to be bioabsorbable and hypothetically have the ability to be drug impregnated may be useful in prosthesis formation herein. These compounds include: poly-1-lactic acid/polyglycolic acid, polyanhydride, and polyphosphate ester. A brief description of each is given below.”

As is also dislosed in U.S. Pat. No. 6,004,346, “Poly-1-lactic acid/polyglycolic acid has been used for many years in the area of bioabsorbable sutures. It is currently available in many forms, i.e., crystals, fibers, blocks, plates, etc . . . ”

As is also dislosed in U.S. Pat. No. 6,004,346, “Another compound which could be used are the polyanhydrides. They are currently being used with several chemotherapy drugs for the treatment of cancerous tumors. These drugs are compounded into the polymer which is molded into a cube-like structure and surgically implanted at the tumor site . . . ”

As is also dislosed in U.S. Pat. No. 6,004,346, “The compound which is preferred is a polyphosphate ester. Polyphosphate ester is a compound such as that disclosed in U.S. Pat. Nos. 5,176,907; 5,194,581; and 5,656,765 issued to Leong which are incorporated herein by reference. Similar to the polyanhydrides, polyphoshate ester is being researched for the sole purpose of drug delivery. Unlike the polyanhydrides, the polyphosphate esters have high molecular weights (600,000 average), yielding attractive mechanical properties. This high molecular weight leads to transparency, and film and fiber properties. It has also been observed that the phosphorous-carbon-oxygen plasticizing effect, which lowers the glass transition temperature, makes the polymer desirable for fabrication.”

As is also dislosed in U.S. Pat. No. 6,004,346, “The basic structure of polyphosphate ester monomer is shown below . . . where P corresponds to Phosphorous, O corresponds to Oxygen, and R and R1 are functional groups. Reaction with water leads to the breakdown of this compound into monomeric phosphates (phosphoric acid) and diols (see below). [Figure] It is the hydrolytic instability of the phosphorous ester bond which makes this polymer attractive for controlled drug release applications. A wide range of controllable degradation rates can be obtained by adjusting the hydrophobicities of the backbones of the polymers and yet assure biodegradability. he functional side groups allow for the chemical linkage of drug molecules to the polymer . . . he drug may also be incorporated into the backbone of the polymer.”

By way of further illustration, and referring to U.S. Pat. No. 6,120,536 (the entire disclosure of which is hereby incorporated by reference into this specification), the polymeric material 14 may comprise a hydrophobic elastomeric material incorporating an amount of biolgocially active material therein for timed release. Some of these elastomeric materials are described at columns 5 and 6 of such patent, wherein it is disclosed that: “The elastomeric materials that form the stent coating underlayers should possess certain properties. Preferably the layers should be of suitable hydrophobic biostable elastomeric materials which do not degrade. Surface layer material should minimize tissue rejection and tissue inflammation and permit encapsulation by tissue adjacent the stent implantation site. Exposed material is designed to reduce clotting tendencies in blood contacted and the surface is preferably modified accordingly. Thus, underlayers of the above materials are preferably provided with a fluorosilicone outer coating layer which may or may not contain imbedded bioactive material, such as heparin. Alternatively, the outer coating may consist essentially of polyethylene glycol (PEG), polysaccharides, phospholipids, or combinations of the foregoing.”

As is also disclosed in U.S. Pat. No. 6,120,536, “Polymers generally suitable for the undercoats or underlayers include silicones (e.g., polysiloxanes and substituted polysiloxanes), polyurethanes, thermoplastic elastomers in general, ethylene vinyl acetate copolymers, polyolefin elastomers, polyamide elastomers, and EPDM rubbers. The above-referenced materials are considered hydrophobic with respect to the contemplated environment of the invention. Surface layer materials include fluorosilicones and polyethylene glycol (PEG), polysaccharides, phospholipids, and combinations of the foregoing.”

As is also dislosed in U.S. Pat. No. 6,120,536, “While heparin is preferred as the incorporated active material, agents possibly suitable for incorporation include antithrobotics, anticoagulants, antibiotics, antiplatelet agents, thorombolytics, antiproliferatives, steroidal and non-steroidal antinflammatories, agents that inhibit hyperplasia and in particular restenosis, smooth muscle cell inhibitors, growth factors, growth factor inhibitors, cell adhesion inhibitors, cell adhesion promoters and drugs that may enhance the formation of healthy neointimal tissue, including endothelial cell regeneration. The positive action may come from inhibiting particular cells (e.g., smooth muscle cells) or tissue formation (e.g., fibromuscular tissue) while encouraging different cell migration (e.g., endothelium) and tissue formation (neointimal tissue) . . . ”.

As is also dislosed in U.S. Pat. No. 6,120,536, “Various combinations of polymer coating materials can be coordinated with biologically active species of interest to produce desired effects when coated on stents to be implanted in accordance with the invention. Loadings of therapeutic materials may vary. The mechanism of incorporation of the biologically active species into the surface coating and egress mechanism depend both on the nature of the surface coating polymer and the material to be incorporated. The mechanism of release also depends on the mode of incorporation. The material may elute via interparticle paths or be administered via transport or diffusion through the encapsulating material itself.”

By way of yet further illustration, and referring to U.S. Pat. No. 6,159,488 (the entire disclosure of which is hereby incorporated by reference into this specification), the polymeric material 14 may be a biopolymer that is non-degradable and is insoluble in biological mediums. Thus, and as is disclosed at column 8 of this patent, “The polymer carrier can be any pharmaceutically acceptable biopolymer that is non-degradable and insoluble in biological mediums, has good stability in a biological environment, has a good adherence to the selected stent, is flexible, and that can be applied as coating to the surface of a stent, either from an organic solvent, or by a melt process. The hydrophilicity or hydrophobicity of the polymer carrier will determine the release rate of halofuginone from the stent surface. Hydrophilic polymers, such as copolymers of hydroxyethyl methacrylate-methyl methacrylate and segmented polyurethane (Hypol), may be used. Hydrophobic coatings such as copolymers of ethylene vinyl acetate, silicone colloidal solutions, and polyurethanes, may be used. The preferred polymers would be those that are rated as medical grade, having good compatibility in contact with blood. The coating may include other antiproliferative agents, such as heparin, steroids and non-steroidal anti-inflammatory agents. To improve the blood compatibility of the coated stent, a hydrophilic coating such as hydromer-hydrophilic polyurethane can be applied.” A material for delivering a biologically active compound comprising a solid carrier material having dissolved and/or dispersed therein at least two biologically active compounds, each of said at least two biologically active compounds having a biologically active nucleus which is common to each of the biologically active compounds, and the at least two biologically active compounds having maximum solubility levels in a single solvent which differ from each other by at least 10% by weight; wherein said solid carrier comprises a biocompatible polymeric material.”

By way of yet further illustration, and referring to claim 1 of U.S. Pat. No. 6,168,801 (the entire disclosure of which is hereby incorporated by reference into this specification), the polymeric material 14 may comprise “A material for delivering a biologically active compound comprising a solid carrier material having dissolved and/or dispersed therein at least two biologically active compounds, each of said at least two biologically active compounds having a biologically active nucleus which is common to each of the biologically active compounds, and the at least two biologically active compounds having maximum solubility levels in a single solvent which differ from each other by at least 10% by weight; wherein said solid carrier comprises a biocompatible polymeric material.”

The device of U.S. Pat. No. 6,168,801 preferably comprises at least two forms of a biologically active ingredient in a single polymeric matrix. Thus, and as is disclosed at column 6 of the patent, “It is contemplated in the practice of the present invention that the combination of the at least two forms of the biologically active ingredient or medically active ingredient in at least a single polymeric carrier can provide release of the active ingredient nucleus common to the at least two forms. The release of the active nucleus can be accomplished by, for example, enzymatic hydrolysis of the forms upon release from the carrier device. Further, the combination of the at least two forms of the biologically active ingredient or medically active ingredient in at least a single polymeric carrier can provide net active ingredient release characterized by the at least simple combination of the two matrix forms described above. This point is illustrated in FIG. 1 which compares the in vitro release of dexamethasone from matrices containing various fractions of two forms of the synthetic steroid dexamethasone, dexamethasone sodium phosphate (DSP; hydrophilic) and dexamethasone acetate (DA; hydrophobic). It is easy to see from these results that the release of dexamethasone acetate (specifically, 100% DA) is slower than all other matrices tested containing some degree or loading of dexamethasone sodium phosphate (hydrophilic). Still further, the resulting active ingredient release from the combined form matrix should be at least more rapid in the early stages of release than the slow single active ingredient component alone. Further still, the cumulative active ingredient release from the combined form matrix should be at least greater in the chronic stages than the fast single active ingredient component. Once again from FIG. 1, the two test matrices containing the greatest amount of dexamethasone sodium phosphate (specifically, 100% DSP, and 75% DSP/25% DA) began to slow in release as pointed out at points “A” and “B”. And further still, the optimal therapeutic release can be designed through appropriate combination of the at least two active biological or medical ingredients in the polymeric carrier material. If as in this example, rapid initial release as well as continuous long term release is desired to achieve a therapeutic goal, the matrix composed of 50% DSP/50% DA would be selected.”

By way of yet further illustration, and referring to claim 1 of U.S. Pat. No. 6,395,300 (the entire disclosure of which is hereby incorporated by reference into this specification), the polymeric material 14 may be a porous polymeric matrix made by a process comprising the steps of: “a) dissolving a drug in a volatile organic solvent to form a drug solution, (b) combining at least one volatile pore forming, agent with the volatile organic drug solution to form an emulsion, suspension, or second solution, and (c) removing the volatile organic solvent and volatile pore forming agent from the emulsion, suspension, or second solution to yield the porous matrix comprising drug, wherein the porous matrix comprising drug has a tap density of less than or equal to 1.0 g/mL or a total surface area of greater than or equal to 0.2 m2/g.”

Referring again to FIG. 1, and to the preferred embodiment depicted therein, the therapeutic agents 18 and/or 20 and/or 22 and/or 24 and/or 26 and/or 28 and/or 30 may be one or more of the drugs disclosed in U.S. Pat. No. 6,624,138, the entire disclosure of which is hereby incorporated by reference into this specification. Thus, and referring to columns 9 et seq. of such patent, “Straub et al. in U.S. Pat. No. 6,395,300 discloses a wide variety of drugs that are useful in the methods and compositions described herein, entire contents of which, including a variety of drugs, are incorporated herein by reference. Drugs contemplated for use in the compositions described in U.S. Pat. No. 6,395,300 and herein disclosed include the following categories and examples of drugs and alternative forms of these drugs such as alternative salt forms, free acid forms, free base forms, and hydrates: analgesics/antipyretics. (e.g., aspirin, acetaminophen, ibuprofen, naproxen sodium, buprenorphine, propoxyphene hydrochloride, propoxyphene napsylate, meperidine hydrochloride, hydromorphone hydrochloide, morphine, oxycodone, codeine, dihydrocodeine bitartrate, pentazocine, hydrocodone bitartrate, levorphanol, diflunisal, trolamine salicylate, nalbuphine hydrochloride, mefenamic acid, butorphanol, choline salicylate, butalbital, phenyltoloxamine citrate, diphenhydramine citrate, methotrimeprazine, cinnamedrine hydrochloride, and meprobamate); antiasthamatics (e.g., ketotifen and traxanox); antibiotics (e.g., neomycin, streptomycin, chloramphenicol, cephalosporin, ampicillin, penicillin, tetracycline, and ciprofloxacin); antidepressants (e.g., nefopam, oxypertine, doxepin, amoxapine, trazodone, amitriptyline, maprotiline, phenelzine, desipramine, nortriptyline, tranylcypromine, fluoxetine, doxepin, imipramine, imipramine pamoate, isocarboxazid, trimipramine, and protriptyline); antidiabetics (e.g., biguanides and sulfonylurea derivatives); antifungal agents (e.g., griseofulvin, ketoconazole, itraconizole, amphotericin B, nystatin, and candicidin); antihypertensive agents (e.g., propanolol, propafenone, oxyprenolol, nifedipine, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, and phentolamine); anti-inflammatories (e.g., (non-steroidal) indomethacin, ketoprofen, flurbiprofen, naproxen, ibuprofen, ramifenazone, piroxicam, (steroidal) cortisone, dexamethasone, fluazacort, celecoxib, rofecoxib, hydrocortisone, prednisolone, and prednisone); antineoplastics (e.g., cyclophosphamide, actinomycin, bleomycin, daunorubicin, doxorubicin, epirubicin, mitomycin, methotrexate, fluorouracil, carboplatin, carmustine (BCNU), methyl-CCNU, cisplatin, etoposide, camptothecin and derivatives thereof, phenesterine, paclitaxel and derivatives thereof, docetaxel and derivatives thereof, vinblastine, vincristine, tamoxifen, and piposulfan); antianxiety agents (e.g., lorazepam, buspirone, prazepam, chlordiazepoxide, oxazepam, clorazepate dipotassium, diazepam, hydroxyzine pamoate, hydroxyzine hydrochloride, alprazolam, droperidol, halazepam, chlormezanone, and dantrolene); immunosuppressive agents (e.g., cyclosporine, azathioprine, mizoribine, and FK506 (tacrolimus)); antimigraine agents (e.g., ergotamine, propanolol, isometheptene mucate, and dichloralphenazone); sedatives/hypnotics (e.g., barbiturates such as pentobarbital, pentobarbital, and secobarbital; and benzodiazapines such as flurazepam hydrochloride, triazolam, and midazolam); antianginal agents (e.g., beta-adrenergic blockers; calcium channel blockers such as nifedipine, and diltiazem; and nitrates such as nitroglycerin, isosorbide dinitrate, pentearythritol tetranitrate, and erythrityl tetranitrate); antipsychotic agents (e.g., haloperidol, loxapine succinate, loxapine hydrochloride, thioridazine, thioridazine hydrochloride, thiothixene, fluphenazine, fluphenazine decanoate, fluphenazine enanthate, trifluoperazine, chlorpromazine, perphenazine, lithium citrate, and prochlorperazine); antimanic agents (e.g., lithium carbonate); antiarrhythmics (e.g., bretylium tosylate, esmolol, verapamil, amiodarone, encainide, digoxin, digitoxin, mexiletine, disopyramide phosphate, procainamide, quinidine sulfate, quinidine gluconate, quinidine polygalacturonate, flecainide acetate, tocainide, and lidocaine); antiarthritic agents (e.g., phenylbutazone, sulindac, penicillanine, salsalate, piroxicam, azathioprine, indomethacin, meclofenamate, gold sodium thiomalate, ketoprofen, auranofin, aurothioglucose, and tolmetin sodium); antigout agents (e.g., colchicine, and allopurinol); anticoagulants (e.g., heparin, heparin sodium, and warfarin sodium); thrombolytic agents (e.g., urokinase, streptokinase, and alteplase); antifibrinolytic agents (e.g., aminocaproic acid); hemorheologic agents (e.g., pentoxifylline); antiplatelet agents (e.g., aspirin); anticonvulsants (e.g., valproic acid, divalproex sodium, phenytoin, phenytoin sodium, clonazepam, primidone, phenobarbitol, carbamazepine, amobarbital sodium, methsuximide, metharbital, mephobarbital, mephenytoin, phensuximide, paramethadione, ethotoin, phenacemide, secobarbitol sodium, clorazepate dipotassium, and trimethadione); antiparkinson agents (e.g., ethosuximide); antihistamines/antipruritics (e.g., hydroxyzine, diphenhydramine, chlorpheniramine, brompheniramine maleate, cyproheptadine hydrochloride, terfenadine, clemastine fumarate, triprolidine, carbinoxamine, diphenylpyraline, phenindamine, azatadine, tripelennamine, dexchlorphenirarnine maleate, methdilazine,; agents useful for calcium regulation (e.g., calcitonin, and parathyroid hormone); antibacterial agents (e.g., amikacin sulfate, aztreonam, chloramphenicol, chloramphenicol palirtate, ciprofloxacin, clindamycin, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, and colistin sulfate); antiviral agents (e.g., interferon alpha, beta or gamma, zidovudine, amantadine hydrochloride, ribavirin, and acyclovir); antimicrobials (e.g., cephalosporins such as cefazolin sodium, cephradine, cefaclor, cephapirin sodium, ceftizoxime sodium, cefoperazone sodium, cefotetan disodium, cefuroxime e azotil, cefotaxime sodium, cefadroxil monohydrate, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, and cefuroxime sodium; penicillins such as ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, aziocillin sodium, carbenicillin indanyl sodium, penicillin G procaine, methicillin sodium, and nafcillin sodium; erythromycins such as erythromycin ethylsuccinate, erythromycin, erythromycin estolate, erythromycin lactobionate, erythromycin stearate, and erythromycin ethylsuccinate; and tetracyclines such as tetracycline hydrochloride, doxycycline hyclate, and minocycline hydrochloride, azithromycin, clarithromycin); anti-infectives (e.g., GM-CSF); bronchodilators (e.g., sympathomimetics such as epinephrine hydrochloride, metaproterenol sulfate, terbutaline sulfate, isoetharine, isoetharine mesylate, isoetharine hydrochloride, albuterol sulfate, albuterol, bitolterolmesylate, isoproterenol hydrochloride, terbutaline sulfate, epinephrine bitartrate, metaproterenol sulfate, epinephrine, and epinephrine bitartrate; anticholinergic agents such as ipratropium bromide; xanthines such as aminophylline, dyphylline, metaproterenol sulfate, and aminophylline; mast cell stabilizers such as cromolyn sodium; inhalant corticosteroids such as beclomethasone dipropionate (BDP), and beclomethasone dipropionate monohydrate; salbutamol; ipratropium bromide; budesonide; ketotifen; salmeterol; xinafoate; terbutaline sulfate; triamcinolone; theophylline; nedocromil sodium; metaproterenol sulfate; albuterol; flunisolide; fluticasone proprionate; steroidal compounds and hormones (e.g., androgens such as danazol, testosterone cypionate, fluoxymesterone, ethyltestosterone, testosterone enathate, methyltestosterone, fluoxymesterone, and testosterone cypionate; estrogens such as estradiol, estropipate, and conjugated estrogens; progestins such as methoxyprogesterone acetate, and norethindrone acetate; corticosteroids such as triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate, methylprednisolone sodium succinate, hydrocortisone sodium succinate, triamcinolone hexacetonide, hydrocortisone, hydrocortisone cypionate, prednisolone, fludrocortisone acetate, paramethasone acetate, prednisolone tebutate, prednisolone acetate, prednisolone sodium phosphate, and hydrocortisone sodium succinate; and thyroid hormones such as levothyroxine sodium); hypoglycemic agents (e.g., human insulin, purified beef insulin, purified pork insulin, glyburide, chlorpropamide, glipizide, tolbutamide, and tolazamide); hypolipidemic agents (e.g., clofibrate, dextrothyroxine sodium, probucol, pravastitin, atorvastatin, lovastatin, and niacin); proteins (e.g., DNase, alginase, superoxide dismutase, and lipase); nucleic acids (e.g., sense or anti-sense nucleic acids encoding any therapeutically useful protein, including any of the proteins described herein); agents useful for erythropoiesis stimulation (e.g., erythropoietin); antiulcer/antireflux agents (e.g., famotidine, cimetidine, and ranitidine hydrochloride); antinauseants/antiemetics (e.g., meclizine hydrochloride, nabilone, prochlorperazine, dimenhydrinate, promethazine hydrochloride, thiethylperazine, and scopolamine);

as well as other drugs useful in the compositions and methods described herein include mitotane, halonitrosoureas, anthrocyclines, ellipticine, ceftriaxone, ketoconazole, ceftazidime, oxaprozin, albuterol, valacyclovir, urofollitropin, famciclovir, flutamide, enalapril, mefformin, itraconazole, buspirone, gabapentin, fosinopril, tramadol, acarbose, lorazepan, follitropin, glipizide, omeprazole, fluoxetine, lisinopril, tramsdol, levofloxacin, zafirlukast, interferon, growth hormone, interleukin, erythropoietin, granulocyte stimulating factor, nizatidine, bupropion, perindopril, erbumine, adenosine, alendronate, alprostadil, benazepril, betaxolol, bleomycin sulfate, dexfenfluramine, diltiazem, fentanyl, flecainid, gemcitabine, glatiramer acetate, granisetron, lamivudine, mangafodipir trisodium, mesalamine, metoprolol fumarate, metronidazole, miglitol, moexipril, monteleukast, octreotide acetate, olopatadine, paricalcitol, somatropin, sumatriptan succinate, tacrine, verapamil, nabumetone, trovafloxacin, dolasetron, zidovudine, finasteride, tobramycin, isradipine, tolcapone, enoxaparin, fluconazole, lansoprazole, terbinafine, pamidronate, didanosine, diclofenac, cisapride, venlafaxine, troglitazone, fluvastatin, losartan, imiglucerase, donepezil, olanzapine, valsartan, fexofenadine, calcitonin, and ipratropium bromide. These drugs are generally considered to be water soluble.”

As is also disclosed in U.S. Pat. No. 6,624,138, “Preferred drugs useful in the present invention may include albuterol, adapalene, doxazosin mesylate, mometasone furoate, ursodiol, amphotericin, enalapril maleate, felodipine, nefazodone hydrochloride, valrubicin, albendazole, conjugated estrogens, medroxyprogesterone acetate, nicardipine hydrochloride, zolpidem tartrate, amidipine besylate, ethinyl estradiol, omeprazole, rubitecan, amlodipine besylate/benazepril hydrochloride, etodolac, paroxetine hydrochloride, paclitaxel, atovaquone, felodipine, podofilox, paricalcitol, betamethasone dipropionate, fentanyl, pramipexole dihydrochloride, Vitamin D3 and related analogues, finasteride, quetiapine fumarate, alprostadil, candesartan, cilexetil, fluconazole, ritonavir, busulfan, carbamazepine, flumazenil, risperidone, carbemazepine, carbidopa, levodopa, ganciclovir, saquinavir, amprenavir, carboplatin, glyburide, sertraline hydrochloride, rofecoxib carvedilol, halobetasolproprionate, sildenafil citrate, celecoxib, chlorthalidone, imiquimod, simvastatin, citalopram, ciprofloxacin, irinotecan hydrochloride, sparfloxacin, efavirenz, cisapride monohydrate, lansoprazole, tamsulosin hydrochloride, mofafinil, clarithromycin, letrozole, terbinafine hydrochloride, rosiglitazone maleate, diclofenac sodium, lomefloxacin hydrochloride, tirofiban hydrochloride, telmisartan, diazapam, loratadine, toremifene citrate, thalidomide, dinoprostone, mefloquine hydrochloride, trandolapril, docetaxel, mitoxantrone hydrochloride, tretinoin, etodolac, triamcinolone acetate, estradiol, ursodiol, nelfinavir mesylate, indinavir, beclomethasone dipropionate, oxaprozin, flutamide, famotidine, nifedipine, prednisone, cefuroxime, lorazepam, digoxin, lovastatin, griseofulvin, naproxen, ibuprofen, isotretinoin, tamoxifen citrate, nimodipine, amiodarone, and alprazolam. Specific non-limiting examples of some drugs that fall under the above categories include paclitaxel, docetaxel and derivatives, epothilones, nitric oxide release agents, heparin, aspirin, coumadin, PPACK, hirudin, polypeptide from angiostatin and endostatin, methotrexate, 5-fluorouracil, estradiol, P-selectin Glycoprotein ligand-1 chimera, abciximab, exochelin, eleutherobin and sarcodictyin, fludarabine, sirolimus, tranilast, VEGF, transforming growth factor (TGF)-beta, Insulin-like growth factor (IGF), platelet derived growth factor (PDGF), fibroblast growth factor (FGF), RGD peptide, beta or gamma ray emitter (radioactive) agents, and dexamethasone, tacrolimus, actinomycin-D, batimastat etc.”

Delivery of Anti-Microtubule Agent

In one embodiment, referring again to FIG. 1, and referring to United States patent 6,689,803 (the entire disclosure of which is hereby incorporated by reference into this specification), one or more of the therapeutic agents 18 and/or 20 and/or 22 and/or 24 and/or 26 and/or 28 and/or 30 may be an anti-microtubule agent. As is disclosed in U.S. Pat. No. 6,689,803 (at columns 5-6), representative anti-microtubule agents include, e.g., . taxanes (e.g., paclitaxel and docetaxel), campothecin, eleutherobin, sarcodictyins, epothilones A and B, discodermolide, deuterium oxide (D2 O), hexylene glycol (2-methyl-2,4-pentanediol), tubercidin (7-deazaadenosine), LY290181 (2-amino-4-(3-pyridyl)-4H-naphtho(1,2-b)pyran-3-cardonitrile), aluminum fluoride, ethylene glycol bis-(succinimidylsuccinate), glycine ethyl ester, nocodazole, cytochalasin B, colchicine, colcemid, podophyllotoxin, benomyl, oryzalin, majusculamide C, demecolcine, methyl-2-benzimidazolecarbamate (MBC), LY195448, subtilisin, 1069C85, steganacin, combretastatin, curacin, estradiol, 2-methoxyestradiol, flavanol, rotenone, griseofulvin, vinca alkaloids, including vinblastine and vincristine, maytansinoids and ansamitocins, rhizoxin, phomopsin A, ustiloxins, dolastatin 10, dolastatin 15, halichondrins and halistatins, spongistatins, cryptophycins, rhazinilam, betaine, taurine, isethionate, HO-221, adociasulfate-2, estramustine, monoclonal anti-idiotypic antibodies, microtubule assembly promoting protein (taxol-like protein, TALP), cell swelling induced by hypotonic (190 mosmol/L) conditions, insulin (100 nmol/L) or glutamine (10 mmol/L), dynein binding, gibberelin, XCHO1 (kinesin-like protein), lysophosphatidic acid, lithium ion, plant cell wall components (e.g., poly-L-lysine and extensin), glycerol buffers, Triton X-100 microtubule stabilizing buffer, microtubule associated proteins (e.g., MAP2, MAP4, tau, big tau, ensconsin, elongation factor-1-alpha (EF-1.alpha.) and E-MAP-115), cellular entities (e.g., histone H1, myelin basic protein and kinetochores), endogenous microtubular structures (e.g., axonemal structures, plugs and GTP caps), stable tubule only polypeptide (e.g., STOP145 and STOP220) and tension from mitotic forces, as well as any analogues and derivatives of any of the above. Within other embodiments, the anti-microtubule agent is formulated to further comprise a polymer.”

The term “anti-micrtubule,” as used in this specification (and in the specification of U.S. Pat. No. 6,689,803), refers to any “. . . protein, peptide, chemical, or other molecule which impairs the function of microtubules, for example, through the prevention or stabilization of polymerization. A wide variety of methods may be utilized to determine the anti-microtubule activity of a particular compound, including for example, assays described by Smith et al. (Cancer Lett 79(2):213-219, 1994) and Mooberry et al., (Cancer Lett. 96(2):261-266, 1995);” see, e.g., lines 13-21 of column 14 of U.S. Pat. No. 6,689,803.

An extensive listing of anti-microtubule agents is provided in columns 14, 15, 16, and 17 of U.S. Pat. No. 6,689,803; and one or more of them may be disposed within polymeric material 14 (see FIG. 1). These anti-microtubule agents include “. . . taxanes (e.g., paclitaxel (discussed in more detail below) and docetaxel) (Schiff et al., Nature 277: 665-667, 1979; Long and Fairchild, Cancer Research 54: 4355-4361, 1994; Ringel and Horwitz, J. Natl. Cancer Inst. 83(4): 288-291, 1991; Pazdur et al., Cancer Treat. Rev. 19(4): 351-386, 1993), campothecin, eleutherobin (e.g., U.S. Pat. No. 5,473,057), sarcodictyins (including sarcodictyin A), epothilones A and B (Bollag et al., Cancer Research 55: 2325-2333, 1995), discodermolide (ter Haar et al., Biochemistry 35: 243-250, 1996), deuterium oxide (D2 O) (James and Lefebvre, Genetics 130(2): 305-314, 1992; Sollott et al., J. Clin. Invest. 95: 1869-1876, 1995), hexylene glycol (2-methyl-2,4-pentanediol) (Oka et al., Cell Struct. Funct. 16(2): 125-134, 1991), tubercidin (7-deazaadenosine) (Mooberry et al., Cancer Lett. 96(2): 261-266, 1995), LY290181 (2-amino-4-(3-pyridyl)-4H-naphtho(1,2-b)pyran-3-cardonitrile) (Panda et al., J. Biol. Chem. 272(12): 7681-7687, 1997; Wood et al., Mol. Pharmacol. 52(3): 437-444, 1997), aluminum fluoride (Song et al., J. Cell. Sci. Suppl. 14: 147-150, 1991), ethylene glycol bis-(succinimidylsuccinate) (Caplow and Shanks, J. Biol. Chem. 265(15): 8935-8941, 1990), glycine ethyl ester (Mejillano et al., Biochemistry 31(13): 3478-3483, 1992), nocodazole (Ding et al., J. Exp. Med. 171(3): 715-727, 1990; Dotti et al., J. Cell Sci. Suppl. 15: 75-84, 1991; Oka et al., Cell Struct. Funct. 16(2): 125-134, 1991; Weimer et al., J. Cell. Biol. 136(1), 71-80, 1997), cytochalasin B (Ulinger et al., Biol. Cell 73(2-3): 131-138, 1991), colchicine and CI 980 (Allen et al., Am. J. Physiol. 261(4 Pt. 1): L315-L321, 1991; Ding et al., J. Exp. Med. 171(3): 715-727, 1990; Gonzalez et al., Exp. Cell. Res. 192(1): 10-15, 1991; Stargell et al., Mol. Cell. Biol. 12(4): 1443-1450, 1992; Garcia et al., Antican. Drugs 6(4): 533-544, 1995), colcemid (Barlow et al., Cell. Motil. Cytoskeleton 19(1): 9-17, 1991; Meschini et al., J. Microsc. 176(Pt. 3): 204-210, 1994; Oka et al., Cell Struct. Funct. 16(2): 125-134, 1991), podophyllotoxin (Ding et al., J. Exp. Med. 171(3): 715-727, 1990), benomyl (Hardwick et al., J. Cell. Biol. 131(3): 709-720, 1995; Shero et al., Genes Dev. 5(4): 549-560, 1991), oryzalin (Stargell et al., Mol. Cell. Biol. 12(4): 1443-1450, 1992), majusculamide C (Moore, J. Ind. Microbiol. 16(2): 134-143, 1996), demecolcine (Van Dolah and Ramsdell, J. Cell. Physiol. 166(1): 49-56, 1996; Wiemer et al., J. Cell. Biol. 136(1): 71-80, 1997), methyl-2-benzimidazolecarbamate (MBC) (Brown et al., J. Cell. Biol. 123(2): 387-403, 1993), LY195448 (Barlow & Cabral, Cell Motil. Cytoskel. 19: 9-17, 1991), subtilisin (Saoudi et al., J. Cell Sci. 108: 357-367, 1995), 1069C85 (Raynaud et al., Cancer Chemother. Pharmacol. 35: 169-173, 1994), steganacin (Hamel, Med. Res. Rev. 16(2): 207-231, 1996), combretastatins (Hamel, Med. Res. Rev. 16(2): 207-231, 1996), curacins (Hamel, Med. Res. Rev. 16(2): 207-231, 1996), estradiol (Aizu-Yokata et al., Carcinogen. 15(9): 1875-1879, 1994), 2-methoxyestradiol (Hamel, Med. Res. Rev. 16(2): 207-231, 1996), flavanols (Hamel, Med. Res. Rev. 16(2): 207-231, 1996), rotenone (Hamel, Med. Res. Rev. 16(2): 207-231, 1996), griseofulvin (Hamel, Med. Res. Rev. 16(2): 207-231; 1996), vinca alkaloids, including vinblastine and vincristine (Ding et al., J. Exp. Med. 171(3): 715-727, 1990; Dirk et al., Neurochem. Res. 15(11): 1135-1139, 1990; Hamel, Med. Res. Rev. 16(2): 207-231, 1996; Illinger et al., Biol. Cell 73(2-3): 131-138, 1991; Wiemer et al., J. Cell. Biol. 136(1): 71-80, 1997), maytansinoids and ansamitocins (Hamel, Med. Res. Rev. 16(2): 207-231, 1996), rhizoxin (Hamel, Med. Res. Rev. 16(2): 207-231, 1996), phomopsin A (Hamel, Med. Res. Rev. 16(2): 207-231, 1996), ustiloxins (Hamel, Med. Res. Rev. 16(2): 207-231, 1996), dolastatin 10 (Hamel, Med Res. Rev. 16(2): 207-231, 1996), dolastatin 15 (Hamel, Med. Res. Rev. 16(2): 207-231, 1996), halichondrins and halistatins (Hamel, Med. Res. Rev. 16(2): 207-231, 1996), spongistatins (Hamel, Med. Res. Rev. 16(2): 207-231, 1996), cryptophycins (Hamel, Med. Res. Rev. 16(2): 207-231, 1996), rhazinilam (Hamel, Med. Res. Rev. 16(2): 207-231, 1996), betaine (Hashimoto et al., Zool. Sci. 1: 195-204, 1984), taurine (Hashimoto et al., Zool. Sci. 1: 195-204, 1984), isethionate (Hashimoto et al., Zool. Sci. 1: 195-204, 1984), HO-221 (Ando et al., Cancer Chemother. Pharmacol. 37: 63-69, 1995), adociasulfate-2 (Sakowicz et al., Science 280: 292-295, 1998), estramustine (Panda et al., Proc. Natl. Acad. Sci. USA 94: 10560-10564, 1997), monoclonal anti-idiotypic antibodies (Leu et al., Proc. Natl. Acad. Sci. USA 91(22): 10690-10694, 1994), microtubule assembly promoting protein (taxol-like protein, TALP) (Hwang et al., Biochem. Biophys. Res. Commun. 208(3): 1174-1180, 1995), cell swelling induced by hypotonic (190 mosmol/L) conditions, insulin (100 nmol/L) or glutamine (10 mmol/L) (Haussinger et al., Biochem. Cell. Biol. 72(1-2): 12-19, 1994), dynein binding (Ohba et al., Biochim. Biophys. Acta 1158(3): 323-332, 1993), gibberelin (Mita and Shibaoka, Protoplasma 119(1/2): 100-109, 1984), XCHO1 kinesin-like protein) (Yonetani et al., Mol. Biol. Cell 7(suppl): 211A, 1996), lysophosphatidic acid (Cook et al., Mol. Biol. Cell 6(suppl): 260A, 1995), lithium ion (Bhattacharyya and Wolff, Biochem. Biophys. Res. Commun. 73(2): 383-390, 1976), plant cell wall components (e.g., poly-L-lysine and extensin) (Akashi et al., Planta 182(3): 363-369, 1990), glycerol buffers (Schilstra et al., Biochem. J. 277(Pt. 3): 839-847, 1991; Farrell and Keates, Biochem. Cell. Biol. 68(11): 1256-1261, 1990; Lopez et al., J. Cell. Biochem. 43(3): 281-291, 1990), Triton X-100 microtubule stabilizing buffer (Brown et al., J. Cell Sci. 104(Pt. 2): 339-352, 1993; Safiejko-Mroczka and Bell, J. Histochem. Cytochem. 44(6): 641-656, 1996), microtubule associated proteins (e.g., MAP2, MAP4, tau, big tau, ensconsin, elongation factor-1-alpha EF-1.alpha.) and E-MAP-115) (Burgess et al., Cell Motil. Cytoskeleton 20(4): 289-300, 1991; Saoudi et al., J. Cell. Sci. 108(Pt. 1): 357-367, 1995; Bulinski and Bossler, J. Cell. Sci. 107(Pt. 10): 2839-2849, 1994; Ookata et al., J. Cell Biol. 128(5): 849-862, 1995; Boyne et al., J. Comp. Neurol. 358(2): 279-293, 1995; Ferreira and Caceres, J. Neurosci. 11(2): 392400, 1991; Thurston et al., Chromosoma 105(1): 20-30, 1996; Wang et al., Brain Res. Mol. Brain Res. 38(2): 200-208, 1996; Moore and Cyr, Mol. Biol. Cell 7(suppl): 221-A, 1996; Masson and Kreis, J. Cell Biol. 123(2), 357-371, 1993), cellular entities (e.g. histone HI, myelin basic protein and kinetochores) (Saoudi et al., J. Cell. Sci. 108(Pt. 1): 357-367, 1995; Simerly et al., J. Cell Biol. 111(4): 1491-1504, 1990), endogenous microtubular structures (e.g., axonemal structures, plugs and GTP caps) (Dye et al., Cell Motil. Cytoskeleton 21(3): 171-186, 1992; Azhar and Murphy, Cell Motil. Cytoskeleton 15(3): 156-161, 1990; Walker et al., J. Cell Biol. 114(1): 73-81, 1991; Drechsel and Kirschner, Curr. Biol. 4(12): 1053-1061, 1994), stable tubule only polypeptide (e.g., STOP145 and STOP220) (Pirollet et al., Biochim. Biophys. Acta 1160(1): 113-119, 1992; Pirollet et al., Biochemistry 31(37): 8849-8855, 1992; Bosc et al., Proc. Natl. Acad. Sci. USA 93(5): 2125-2130, 1996; Margolis et al., EMBO J. 9(12): 4095-4102, 1990) and tension from mitotic forces (Nicklas and Ward, J. Cell Biol. 126(5): 1241-1253, 1994), as well as any analogues and derivatives of any of the above. Such compounds can act by either depolymerizing microtubules (e.g., colchicine and vinblastine), or by stabilizing microtubule formation (e.g., paclitaxel).”

U.S. Pat. No. 6,689,803 also discloses (at columns 16 and 17 that, “Within one preferred embodiment of the invention, the therapeutic agent is paclitaxel, a compound which disrupts microtubule formation by binding to tubulin to form abnormal mitotic spindles. Briefly, paclitaxel is a highly derivatized diterpenoid (Wani et al., J. Am. Chem. Soc. 93:2325, 1971) which has been obtained from the harvested and dried bark of Taxus brevifolia (Pacific Yew) and Taxomyces Andreanae and Endophytic Fungus of the Pacific Yew (Stierle et al., Science 60:214-216-1993). “Paclitaxel” (which should be understood herein to include prodrugs, analogues and derivatives such as, for example, TAXOL®, TAXOTERE®, Docetaxel, 10-desacetyl analogues of paclitaxel and 3′N-desbenzoyl-3′N-t-butoxy carbonyl analogues of paclitaxel) may be readily prepared utilizing techniques known to those skilled in the art (see e.g., Schiff et al., Nature 277:665-667, 1979; Long and Fairchild, Cancer Research 54:4355-4361, 1994; Ringel and Horwitz, J. Natl. Cancer Inst. 83(4):288-291, 1991; Pazdur et al., Cancer Treat. Rev. 19(4):351-386, 1993; WO 94/07882; WO 94/07881; WO 94/07880; WO 94/07876; WO 93/23555; WO 93/10076; WO94/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52):9709-9712, 1994; J. Med. Chem. 35:4230-4237, 1992; J. Med. Chem. 34:992-998, 1991; J. Natural Prod. 57(10):1404-1410, 1994; J. Natural Prod. 57(11):1580-1583, 1994; J. Am. Chem. Soc. 110:6558-6560, 1988), or obtained from a variety of commercial sources, including for example, Sigma Chemical Co., St. Louis, Mo. (T7402—from Taxus brevifolia).”

As is also disclosed in U.S. Pat. No. 6,689,893, “Representative examples of such paclitaxel derivatives or analogues include 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol (from 10-deacetylbaccatin III), phosphonooxy and carbonate derivatives of taxol, taxol 2′,7-di(sodium 1,2-benzenedicarboxylate, 10-desacetoxy-11,12-dihydrotaxol-10,12(18)-diene derivatives, 10-desacetoxytaxol, Protaxol(2′- and/or 7-0-ester derivatives), (2′- and/or 7-0-carbonate derivatives), asymmetric synthesis of taxol side chain, fluoro taxols, 9-deoxotaxane, (13-acetyl-9-deoxobaccatine III, 9-deoxotaxol, 7-deoxy-9-deoxotaxol, 10-desacetoxy-7-deoxy-9-deoxotaxol, Derivatives containing hydrogen or acetyl group and a hydroxy and tert-butoxycarbonylamino, sulfonated 2′-acryloyltaxol and sulfonated 2′-O-acyl acid taxol derivatives, succinyltaxol, 2′-.gamma.-aminobutyryltaxol formate, 2′-acetyl taxol, 7-acetyl taxol, 7-glycine carbamate taxol, 2′-OH-7-PEG(5000)carbamate taxol, 2′-benzoyl and 2′,7-dibenzoyl taxol derivatives, other prodrugs (2′-acetyl taxol; 2′,7-diacetyltaxol; 2′succinyltaxol; 2′-(beta-alanyl)-taxol); 2′gamma-aminobutyryltaxol formate; ethylene glycol derivatives of 2′-succinyltaxol; 2′-glutaryltaxol; 2′-(N,N-dimethylglycyl)taxol; 2′-(2-(N,N-dimethylamino)propionyl)taxol; 2′orthocarboxybenzoyl taxol; 2′aliphatic carboxylic acid derivatives of taxol, Prodrugs {2′(N,N-diethylaminopropionyl)taxol, 2′(N,N-dimethylglycyl)taxol, 7(N,N-dimethylglycyl)taxol, 2′,7-di-(N,N-dimethylglycyl)taxol, 7(N,N-diethylaminopropionyl)taxol, 2′,7-di(N,N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-isoleucyl)taxol, 2′-(L-valyl)taxol, 7-(L-valyl)taxol, 2′7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, Taxol analogs with modified phenylisoserine side chains, taxotere, (N-debenzoyl-N-tert-(butoxycaronyl)-10-deacetyltaxol, and taxanes (e.g., baccatin III, cephalomannine, 10-deacetylbaccatin III, brevifoliol, yunantaxusin and taxusin).”

At columns 17, 18, 19, and 20 of U.S. Pat. No. 6,689,803, several “polymeric carriers” are described. One or more of these “polymeric carriers” may be used as the polymeric material 14. Thus, and referring to columns 17-20 of such United States patent, “ . . . a wide variety of polymeric carriers may be utilized to contain and/or deliver one or more of the therapeutic agents discussed above, including for example both biodegradable and non-biodegradable compositions. Representative examples of biodegradable compositions include albumin, collagen, gelatin, hyaluronic acid, starch, cellulose (methylcellulose, hydroxypropylcellulose, hydroxypropylmethylcellulose, hydroxyethylcellulose, carboxymethylcellulose, cellulose acetate phthalate, cellulose acetate succinate, hydroxypropylmethylcellulose phthalate), casein, dextrans, polysaccharides, fibrinogen, poly(D,L lactide), poly(D,L-lactide-co-glycolide), poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids) and their copolymers (see generally, Illum, L., Davids, S. S. (eds.) “Polymers in Controlled Drug Delivery” Wright, Bristol, 1987; Arshady, J. Controlled Release 17:1-22, 1991; Pitt, Int. J. Phar. 59:173-196, 1990; Holland et al., J. Controlled Release 4:155-0180, 1986). Representative examples of nondegradable polymers include poly(ethylene-vinyl acetate) (“EVA”) copolymers, silicone rubber, acrylic polymers (polyacrylic acid, polymethylacrylic acid, polymethylmethacrylate, polyalkylcynoacrylate), polyethylene, polyproplene, polyamides (nylon 6,6), polyurethane, poly(ester urethanes), poly(ether urethanes), poly(ester-urea), polyethers (poly(ethylene oxide), poly(propylene oxide), Pluronics and poly(tetramethylene glycol)), silicone rubbers and vinyl polymers (polyvinylpyrrolidone, poly(vinyl alcohol), poly(vinyl acetate phthalate). Polymers may also be developed which are either anionic (e.g. alginate, carrageenin, carboxymethyl cellulose and poly(acrylic acid), or cationic (e.g., chitosan, poly-L-lysine, polyethylenimine, and poly (allyl amine)) (see generally, Dunn et al., J. Applied Polymer Sci. 50:353-365, 1993; Cascone et al., J. Materials Sci.: Materials in Medicine 5:770-774, 1994; Shiraishi et al., Biol. Pharm. Bull. 16(11):1164-1168, 1993; Thacharodi and Rao, Int'l J. Pharm. 120:115-118, 1995; Miyazaki et al., Int'l J. Pharm. 118:257-263, 1995). Particularly preferred polymeric carriers include poly(ethylenevinyl acetate), poly (D,L-lactic acid) oligomers and polymers, poly (L-lactic acid) oligomers and polymers, poly (glycolic acid), copolymers of lactic acid and glycolic acid, poly (caprolactone), poly (valerolactone), polyanhydrides, copolymers of poly (caprolactone) or poly (lactic acid) with a polyethylene glycol (e.g., MePEG), and blends thereof.”

As is also disclosed in U.S. Pat. No. 6,689,893, “Polymeric carriers can be fashioned in a variety of forms, with desired release characteristics and/or with specific desired properties. For example, polymeric carriers may be fashioned to release a therapeutic agent upon exposure to a specific triggering event such as pH (see e.g., Heller et al., “Chemically Self-Regulated Drug Delivery Systems,” in Polymers in Medicine III, Elsevier Science Publishers B. V., Amsterdam, 1988, pp. 175-188; Kang et al., J. Applied Polymer Sci. 48:343-354, 1993; Dong et al., J. Controlled Release 19:171-178, 1992; Dong and Hoffmnan, J. Controlled Release 15:141-152, 1991; Kim et al., J. Controlled Release 28:143-152, 1994; Cornejo-Bravo et al., J. Controlled Release 33:223-229, 1995; Wu and Lee, Pharm. Res. 10(10):1544-1547, 1993; Serres et al., Pharm. Res. 13(2):196-201, 1996; Peppas, “Fundamentals of pH- and Temperature-Sensitive Delivery Systems,” in Gumy et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers I, Springer-Verlag, Berlin). Representative examples of pH-sensitive polymers include poly(acrylic acid) and its derivatives (including for example, homopolymers such as poly(aminocarboxylic acid); poly(acrylic acid); poly(methyl acrylic acid), copolymers of such homopolymers, and copolymers of poly(acrylic acid) and acrylmonomers such as those discussed above. Other pH sensitive polymers include polysaccharides such as cellulose acetate phthalate; hydroxypropylmethylcellulose phthalate; hydroxypropylmethylcellulose acetate succinate; cellulose acetate trimellilate; and chitosan. Yet other pH sensitive polymers include any mixture of a pH sensitive polymer and a water soluble polymer.”

As is also disclosed in U.S. Pat. No. 6,689,893, “Likewise, polymeric carriers can be fashioned which are temperature sensitive (see e.g., Chen et al., “Novel Hydrogels of a Temperature-Sensitive Pluronic Grafted to a Bioadhesive Polyacrylic Acid Backbone for Vaginal Drug Delivery,” in Proceed. Intern. Symp. Control. Rel. Bioact. Mater. 22:167-168, Controlled Release Society, Inc., 1995; Okano, “Molecular Design of Stimuli-Responsive Hydrogels for Temporal Controlled Drug Delivery,” in Proceed. Intern. Symp. Control. Rel. Bioact. Mater. 22:111-112, Controlled Release Society, Inc., 1995; Johnston et al., Pharm. Res. 9(3):425-433, 1992; Tung, Int'l J. Pharm. 107:85-90, 1994; Harsh and Gehrke, J. Controlled Release 17:175-186, 1991; Bae et al., Pharm. Res. 8(4):531-537, 1991; Dinarvand and D'Emanuele, J. Controlled Release 36:221-227, 1995; Yu and Grainger, “Novel Thermo-sensitive Amphiphilic Gels: Poly N-isopropylacrylamide-co-sodium acrylate-co-n-N-alkylacrylamide Network Synthesis and Physicochemical Characterization,” Dept. of Chemical & Biological Sci., Oregon Graduate Institute of Science & Technology, Beaverton, Oreg., pp. 820-821; Zhou and Smid, “Physical Hydrogels of Associative Star Polymers,” Polymer Research Institute, Dept. of Chemistry, College of Environmental Science and Forestry, State Univ. of New York, Syracuse, N.Y., pp. 822-823; Hoffman et al., “Characterizing Pore Sizes and Water ‘Structure’ in Stimuli-Responsive Hydrogels,” Center for Bioengineering, Univ. of Washington, Seattle, Wash., p. 828; Yu and Grainger, “Thermo-sensitive Swelling Behavior in Crosslinked N-isopropylacrylamide Networks: Cationic, Anionic and Ampholytic Hydrogels,” Dept. of Chemical & Biological Sci., Oregon Graduate Institute of Science & Technology, Beaverton, Oreg., pp. 829-830; Kim et al., Pharm. Res. 9(3):283-290, 1992; Bae et al., Pharm. Res. 8(5):624-628, 1991; Kono et al., J. Controlled Release 30:69-75, 1994; Yoshida et al., J. Controlled Release 32:97-102. 1994; Okano et al., J. Controlled Release 36:125-133, 1995; Chun and Kim, J. Controlled Release 38:39-47, 1996; D'Emanuele and Dinarvand, Int'l J. Pharm. 118:237-242, 1995; Katono et al., J. Controlled Release 16:215-228, 1991; Hoffman, “Thermally Reversible Hydrogels Containing Biologically Active Species,” in Migliaresi et al. (eds.), Polymers in Medicine III, Elsevier Science Publishers B. V., Amsterdam, 1988, pp. 161-167; Hoffman, “Applications of Thermally Reversible Polymers and Hydrogels in Therapeutics and Diagnostics,” in Third International Symposium on Recent Advances in Drug Delivery Systems, Salt Lake City, Utah, Feb. 24-27, 1987, pp. 297-305; Gutowska et al., J. Controlled Release 22:95-104, 1992; Palasis and Gehrke, J. Controlled Release 18:1-12, 1992; Paavola et al., Pharm. Res. 12(12): 1997-2002, 1995).” In one embodiment, the polymeric material 14 is temperature sensitive.

As is also disclosed in U.S. Pat. No. 6,689,893, “Representative examples of thermogelling polymers, and their gelatin temperature (LCST (° C.)) include homopolymers such as poly(-methyl-N-n-propylacrylamide), 19.8; poly(N-n-propylacrylamide), 21.5; poly(N-methyl-N-isopropylacrylamide), 22.3; poly(N-n-propylmethacrylamide), 28.0; poly(N-isopropylacrylamide), 30.9; poly(N,n-diethylacrylamide), 32.0; poly(N-isopropylmethacrylamide), 44.0; poly(N-cyclopropylacrylamide), 45.5; poly(N-ethylmethyacrylamide), 50.0; poly(N-methyl-N-ethylacrylamide), 56.0; poly(N-cyclopropylmethacrylamide), 59.0; poly(N-ethylacrylamide), 72.0. Moreover thermogelling polymers may be made by preparing copolymers between (among) monomers of the above, or by combining such homopolymers with other water soluble polymers such as acrylmonomers (e.g., acrylic acid and derivatives thereof such as methylacrylic acid, acrylate and derivatives thereof such as butyl methacrylate, acrylamide, and N-n-butyl acrylamide).”

As is also disclosed in U.S. Pat. No. 6,689,893, “Other representative examples of thermogelling polymers include cellulose ether derivatives such as hydroxypropyl cellulose, 41° C.; methyl cellulose, 55° C.; hydroxypropylmethyl cellulose, 66° C.; and ethylhydroxyethyl cellulose, and Pluronics such as F-127, 10-15° C.; L-122, 19° C.; L-92, 26° C.; L-81, 20° C.; and L-61, 20° C.”

As is also disclosed in U.S. Pat. No. 6,689,893, “Preferably, therapeutic compositions of the present invention are fashioned in a manner appropriate to the intended use. Within certain aspects of the present invention, the therapeutic composition should be biocompatible, and release one or more therapeutic agents over a period of several days to months. For example, “quick release” or “burst” therapeutic compositions are provided that release greater than 10%, 20%, or 25% (w/v) of a therapeutic agent (e.g., paclitaxel) over a period of 7 to 10 days. Such “quick release” compositions should, within certain embodiments, be capable of releasing chemotherapeutic levels (where applicable) of a desired agent. Within other embodiments, “low release” therapeutic compositions are provided that release less than 1% (w/v) of a therapeutic agent over a period of 7 to 10 days. Further, therapeutic compositions of the present invention should preferably be stable for several months and capable of being produced and maintained under sterile conditions.”

Nanomagnetic Particles 32

Referring again to FIGS. 1 and 1A, and to the preferred embodiment depicted therein, the sealed container 12 is preferably comprised of one or more nanomagentic particles 32. Furthermore, in the preferred embodiment depicted in FIGS. 1 and 1A, a film 16 is disposed around sealed container 12, and this film is also preferably comprised of nanomagnetic particles 32 (not shown for the sake of simplicity of representation).

These nanomagnetic particles are described in “case XW-672,” filed on Mar. 24, 2004 by Xingwu Wang and Howard J. Greenwald as U.S. Pat. No. application Ser. No. 10/808,618; the entire disclosure of this U.S. Pat. No. application is hereby incorporated by reference into this specification.

In the remainder of this section of the patent application, reference will be had to some of the disclosure of U.S. Ser. No. 10/808,618 to help describe the nanomagnetic particles 32.

In one embodiment of the invention depicted in FIG. 1, and disposed within sealed container 12, there is collection of nanomagentic particles 32 with an average particle size of less than about 100 nanometers. The average coheence length between adjacent nanomagnetic particles is preferably less than about 100 nanometers. The nanomagnetic particles 32 preferably have a saturation magentization of from about 2 to about 3000 electromagnetic units per cubic centimeter, and a phase transition temperature of from about 40 to about 200 degrees Celsius.

Some similar nanomagnetic particles are disclosed in applicants' U.S. Pat. No. 6,502,972, which describes and claims a magnetically shielded conductor assembly comprised of a first conductor disposed within an insulating matrix, and a layer comprised of nanomagnetic material disposed around said first conductor, provided that such nanomagnetic material is not contiguous with said first conductor. In this assembly, the first conductor has a resistivity at 20 degrees Centigrade of from about 1 to about 100 micro ohm-centimeters, the insulating matrix is comprised of nano-sized particles wherein at least about 90 weight percent of said particles have a maximum dimension of from about 10 to about 100 nanometers, the insulating matrix has a resistivity of from about 1,000,000,000 to about 10,000,000,000,000 ohm-centimeter, the nanomagnetic material has an average particle size of less than about 100 nanometers, the layer of nanomagnetic material has a saturation magnetization of from about 200 to about 26,000 Gauss and a thickness of less than about 2 microns, and the magnetically shielded conductor assembly is flexible, having a bend radius of less than 2 centimeters. The entire disclosure of this U.S. Pat. No. is hereby incorporated by reference into this specification.

The nanomagnetic film disclosed in U.S. Pat. No. 6,506,972 may be used to shield medical devices (such as the sealed container 12 of FIG. 1) from external electromagnetic fields; and, when so used, it provides a certain degree of shielding. The medical devices so shielded may be coated with one or more drug formulations, as described elsewhere in this specification..

FIG. 2 is a schematic illustration of one process of the invention that may be used to make nanomagnetic material. This FIG. 2 is similar in many respects to the FIG. 1 of U.S. Pat. No. 5,213,851, the entire disclosure of which is hereby incorporated by reference into this specification.

Referring to FIG. 2, and in the preferred embodiment depicted therein, it is preferred that the reagents charged into misting chamber 11 will be sufficient to form a nano-sized ferrite in the process. The term ferrite, as used in this specification, refers to a material that exhibits ferromagnetism. Ferromagnetism is a property, exhibited by certain metals, alloys, and compounds of the transition (iron group) rare earth and actinide elements, in which the internal magnetic moments spontaneously organize in a common direction; ferromagnetism gives rise to a permeability considerably greater than that of vacuum and to magnetic hysteresis. See, e.g, page 706 of Sybil B. Parker's “McGraw-Hill Dictionary of Scientific and Technical Terms,” Fourth Edition (McGraw-Hill Book Company, New York, N.Y., 1989).

As will be apparent to those skilled in the art, in addition to making nano-sized ferrites by the process depicted in FIG. 2, one may also make other nano-sized materials such as, e.g., nano-sized nitrides and/or nano-sized oxides containing moieties A, B, and C, as is described elsewhere in this specification. For the sake of simplicity of description, and with regard to FIG. 2, a discussion will be had regarding the preparation of ferrites, it being understood that, e.g., other materials may also be made by such process.

Referring again to FIG. 2, and to the production of ferrites by such process, in one embodiment, the ferromagnetic material contains Fe₂ O₃. See, for example, U.S. Pat. No. 3,576,672 of Harris et al., the entire disclosure of which is hereby incorporated by reference into this specification. As will be apparent, the corresponding nitrides also may be made.

In one embodiment, the ferromagnetic material contains garnet. Pure iron garnet has the formula M₃Fe₅O₁₂; see, e.g., pages 65-256 of Wilhelm H. Von Aulock's “Handbook of Microwave Ferrite Materials” (Academic Press, New York, 1965). Garnet ferrites are also described, e.g., in U.S. Pat. No. 4,721,547, the disclosure of which is hereby incorporated by reference into this specification. As will be apparent, the corresponding nitrides also may be made.

In another embodiment, the ferromagnetic material contains a spinel ferrite. Spinel ferrites usually have the formula MFe₂O₄, wherein M is a divalent metal ion and Fe is a trivalent iron ion. M is typically selected from the group consisting of nickel, zinc, magnesium, manganese, and like. These spinel ferrites are well known and are described, for example, in U.S. Pat. Nos. 5,001,014, 5,000,909, 4,966,625, 4,960,582, 4,957,812, 4,880,599, 4,862,117, 4,855,205, 4,680,130, 4,490,268, 3,822,210, 3,635,898, 3,542,685, 3,421,933, and the like. The disclosure of each of these patents is hereby incorporated by reference into this specification. Reference may also be had to pages 269-406 of the Von Aulock book for a discussion of spinel ferrites. As will be apparent, the corresponding nitrides also may be made.

In yet another embodiment, the ferromagnetic material contains a lithium ferrite. Lithium ferrites are often described by the formula (Li_{0.5}Fe_{0.5})2+(Fe₂)3+0₄. Some illustrative lithium ferrites are described on pages 407-434 of the aforementioned Von Aulock book and in U.S. Pat. Nos. 4,277,356, 4,238,342, 4,177,438, 4,155,963, 4,093,781, 4,067,922, 3,998,757, 3,767,581, 3,640,867, and the like. The disclosure of each of these patents is hereby incorporated by reference into this specification. As will be apparent, the corresponding nitrides also may be made.

In yet another embodiment, the ferromagnetic material contains a hexagonal ferrite. These ferrites are well known and are disclosed on pages 451-518 of the Von Aulock book and also in U.S. Pat. Nos. 4,816,292, 4,189,521, 5,061,586, 5,055,322, 5,051,201, 5,047,290, 5,036,629, 5,034,243, 5,032,931, and the like. The disclosure of each of these patents is hereby incorporated by reference into this specification. As will be apparent, the corresponding nitrides also may be made.

In yet another embodiment, the ferromagnetic material contains one or more of the moieties A, B, and C disclosed in the phase diagram disclosed elsewhere in this specification and discussed elsewhere in this specification.

Referring again to FIG. 2, and in the preferred embodiment depicted therein, it will be appreciated that the solution 9 will preferably comprise reagents necessary to form the required magnetic material. For example, in one embodiment, in order to form the spinel nickel ferrite of the formula NiFe₂O₄, the solution should contain nickel and iron, which may be present in the form of nickel nitrate and iron nitrate. By way of further example, one may use nickel chloride and iron chloride to form the same spinel. By way of further example, one may use nickel sulfate and iron sulfate.

It will be apparent to skilled chemists that many other combinations of reagents, both stoichiometric and nonstoichiometric, may be used in applicants' process to make many different magnetic materials.

In one preferred embodiment, the solution 9 contains the reagent needed to produce a desired ferrite in stoichiometric ratio. Thus, to make the NiFe₂O₄ ferrite in this embodiment, one mole of nickel nitrate may be charged with every two moles of iron nitrate.

In one embodiment, the starting materials are powders with purities exceeding 99 percent.

In one embodiment, compounds of iron and the other desired ions are present in the solution in the stoichiometric ratio.

In one preferred embodiment, ions of nickel, zinc, and iron are present in a stoichiometric ratio of 0.5/0.5/2.0, respectively. In another preferred embodiment, ions of lithium and iron are present in the ratio of 0.5/2.5. In yet another preferred embodiment, ions of magnesium and iron are present in the ratio of 1.0/2.0. In another embodiment, ions of manganese and iron are present in the ratio 1.0/2.0. In yet another embodiment, ions of yttrium and iron are present in the ratio of 3.0/5.0. In yet another embodiment, ions of lanthanum, yttrium, and iron are present in the ratio of 0.5/2.5/5.0. In yet another embodiment, ions of neodymium, yttrium, gadolinium, and iron are present in the ratio of 1.0/1.07/0.93/5.0, or 1.0/1.1/0.9/5.0, or 1/1.12/0.88/5.0. In yet another embodiment, ions of samarium and iron are present in the ratio of 3.0/5.0. In yet another embodiment, ions of neodymium, samarium, and iron are present in the ratio of 0.1/2.9/5.0, or 0.25/2.75/5.0, or 0.375/2.625/5.0. In yet another embodiment, ions of neodymium, erbium, and iron are present in the ratio of 1.5/1.5/5.0. In yet another embodiment, samarium, yttrium, and iron ions are present in the ratio of 0.51/2.49/5.0, or 0.84/2.16/5.0, or 1.5/1.5/5.0. In yet another embodiment, ions of yttrium, gadolinium, and iron are present in the ratio of 2.25/0.75/5.0, or 1.5/1.5/5.0, or 0.75/2.25/5.0. In yet another embodiment, ions of terbium, yttrium, and iron are present in the ratio of 0.8/2.2/5.0, or 1.0/2.0/5.0. In yet another embodiment, ions of dysprosium, aluminum, and iron are present in the ratio of 3/x/5-x, when x is from 0 to 1.0. In yet another embodiment, ions of dysprosium, gallium, and iron are also present in the ratio of 3/x/5-x. In yet another embodiment, ions of dysprosium, chromium, and iron are also present in the ratio of 3/x/5-x.

The ions present in the solution, in one embodiment, may be holmium, yttrium, and iron, present in the ratio of z/3-z/5.0, where z is from about 0 to 1.5.

The ions present in the solution may be erbium, gadolinium, and iron in the ratio of 1.5/1.5/5.0. The ions may be erbium, yttrium, and iron in the ratio of 1.5/1.5/1.5, or 0.5/2.5/5.0.

The ions present in the solution may be thulium, yttrium, and iron, in the ratio of 0.06/2.94/5.0.

The ions present in the solution may be ytterbium, yttrium, and iron, in the ratio of 0.06/2.94/5.0.

The ions present in the solution may be lutetium, yttrium, and iron in the ratio of y/3-y/5.0, wherein y is from 0 to 3.0.

The ions present in the solution may be iron, which can be used to form Fe₆O₈ (two formula units of Fe₃O₄). The ions present may be barium and iron in the ratio of 1.0/6.0, or 2.0/8.0. The ions present may be strontium and iron, in the ratio of 1.0/12.0. The ions present may be strontium, chromium, and iron in the ratio of 1.0/1.0/10.0, or 1.0/6.0/6.0. The ions present may be suitable for producing a ferrite of the formula (Meₓ)₃+Ba₁₋ₓFe₁₂O₁₉, wherein Me is a rare earth selected from the group consisting of lanthanum, promethium, neodymium, samarium, europium, and mixtures thereof.

The ions present in the solution may contain barium, either lanthanum or promethium, iron, and cobalt in the ratio of 1-a/a/12-a/a, wherein a is from 0.0 to 0.8.

The ions present in the solution may contain barium, cobalt, titanium, and iron in the ratio of 1.0/b/b/12-2b, wherein b is from 0.0 to 1.6.

The ions present in the solution may contain barium, nickel or cobalt or zinc, titanium, and iron in the ratio of 1.0/c/c/12-2c, wherein c is from 0.0 to 1.5.

The ions present in the solution may contain barium, iron, iridium, and zinc in the ratio of 1.0/12-2d/d/d, wherein d is from 0.0 to 0.6.

The ions present in the solution may contain barium, nickel, gallium, and iron in the ratio of 1.0/2.0/7.0/9.0, or 1.0/2.0/5.0/11.0. Alternatively, the ions may contain barium, zinc, gallium or aluminum, and iron in the ratio of 1.0/2.0/3.0/13.0.

Each of these ferrites is well known to those in the ferrite art and is described, e.g., in the aforementioned Von Aulock book.

The ions described above are preferably available in solution 9 in water-soluble form, such as, e.g., in the form of water-soluble salts. Thus, e.g., one may use the nitrates or the chlorides or the sulfates or the phosphates of the cations. Other anions which form soluble salts with the cation(s) also may be used.

Alternatively, one may use salts soluble in solvents other than water. Some of these other solvents which may be used to prepare the material include nitric acid, hydrochloric acid, phosphoric acid, sulfuric acid, and the like. As is well known to those skilled in the art, many other suitable solvents may be used; see, e.g., J. A. Riddick et al., “Organic Solvents, Techniques of Chemistry,” Volume II, 3rd edition (Wiley-Interscience, New York, N.Y., 1970).

In one preferred embodiment, where a solvent other than water is used, each of the cations is present in the form of one or more of its oxides. For example, one may dissolve iron oxide in nitric acid, thereby forming a nitrate. For example, one may dissolve zinc oxide in sulfuric acid, thereby forming a sulfate. One may dissolve nickel oxide in hydrochloric acid, thereby forming a chloride. Other means of providing the desired cation(s) will be readily apparent to those skilled in the art.

In general, as long as the desired cation(s) are present in the solution, it is not significant how the solution was prepared.

In general, one may use commercially available reagent grade materials. Thus, by way of illustration and not limitation, one may use the following reagents available in the 1988-1989 Aldrich catalog (Aldrich Chemical Company, Inc., Milwaukee, Wis.): barium chloride, catalog number 31,866-3; barium nitrate, catalog number 32,806-5; barium sulfate, catalog number 20,276-2; strontium chloride hexhydrate, catalog number 20,466-3; strontium nitrate, catalog number 20,449-8; yttrium chloride, catalog number 29,826-3; yttrium nitrate tetrahydrate, catalog number 21,723-9; yttrium sulfate octahydrate, catalog number 20,493-5. This list is merely illustrative, and other compounds that can be used will be readily apparent to those skilled in the art. Thus, any of the desired reagents also may be obtained from the 1989-1990 AESAR catalog (Johnson Matthey/AESAR Group, Seabrook, N.H.), the 1990/1991 Alfa catalog (Johnson Matthey/Alfa Products, Ward Hill, Mass.), the Fisher 88 catalog (Fisher Scientific, Pittsburgh, Pa.), and the like.

As long as the metals present in the desired ferrite material are present in solution 9 in the desired stoichiometry, it does not matter whether they are present in the form of a salt, an oxide, or in another form. In one embodiment, however, it is preferred to have the solution contain either the salts of such metals, or their oxides.

The solution 9 of the compounds of such metals preferably will be at a concentration of from about 0.01 to about 1,000 grams of said reagent compounds per liter of the resultant solution. As used in this specification, the term liter refers to 1,000 cubic centimeters.

In one embodiment, it is preferred that solution 9 have a concentration of from about 1 to about 300 grams per liter and, preferably, from about 25 to about 170 grams per liter. It is even more preferred that the concentration of said solution 9 be from about 100 to about 160 grams per liter. In an even more preferred embodiment, the concentration of said solution 9 is from about 140 to about 160 grams per liter.

In one preferred embodiment, aqueous solutions of nickel nitrate, and iron nitrate with purities of at least 99.9 percent are mixed in the molar ratio of 1:2 and then dissolved in distilled water to form a solution with a concentration of 150 grams per liter.

In one preferred embodiment, aqueous solutions of nickel nitrate, zinc nitrate, and iron nitrate with purities of at least 99.9 percent are mixed in the molar ratio of 0.5:0.5:2 and then dissolved in distilled water to form a solution with a concentration of 150 grams per liter.

In one preferred embodiment, aqueous solutions of zinc nitrate, and iron nitrate with purities of at least 99.9 percent are mixed in the molar ratio of 1:2 and then dissolved in distilled water to form a solution with a concentration of 150 grams per liter.

In one preferred embodiment, aqueous solutions of nickel chloride, and iron chloride with purities of at least 99.9 percent are mixed in the molar ratio of 1:2 and then dissolved in distilled water to form a solution with a concentration of 150 grams per liter.

In one preferred embodiment, aqueous solutions of nickel chloride, zinc chloride, and iron chloride with purities of at least 99.9 percent are mixed in the molar ratio of 0.5:0.5:2 and then dissolved in distilled water to form a solution with a concentration of 150 grams per liter.

In one preferred embodiment, aqueous solutions of zinc chloride, and iron chloride with purities of at least 99.9 percent are mixed in the molar ratio of 1:2 and then dissolved in distilled water to form a solution with a concentration of 150 grams per liter.

In one embodiment, mixtures of chlorides and nitrides may be used. Thus, for example, in one preferred embodiment, the solution is comprised of both iron chloride and nickel nitrate in the molar ratio of 2.0/1.0.

Referring again to FIG. 2, and to the preferred embodiment depicted therein, the solution 9 in misting chamber 11 is preferably caused to form into an aerosol, such as a mist.

The term aerosol, as used in this specification, refers to a suspension of ultramicroscopic solid or liquid particles in air or gas, such as smoke, fog, or mist. See, e.g., page 15 of “A dictionary of mining, mineral, and related terms,” edited by Paul W. Thrush (U.S. Department of the Interior, Bureau of Mines, 1968), the disclosure of which is hereby incorporated by reference into this specification.

As used in this specification, the term mist refers to gas-suspended liquid particles which have diameters less than 10 microns.

The aerosol/mist consisting of gas-suspended liquid particles with diameters less than 10 microns may be produced from solution 9 by any conventional means that causes sufficient mechanical disturbance of said solution. Thus, one may use mechanical vibration. In one preferred embodiment, ultrasonic means are used to mist solution 9. As is known to those skilled in the art, by varying the means used to cause such mechanical disturbance, one can also vary the size of the mist particles produced.

As is known to those skilled in the art, ultrasonic sound waves (those having frequencies above 20,000 hertz) may be used to mechanically disturb solutions and cause them to mist. Thus, by way of illustration, one may use the ultrasonic nebulizer sold by the DeVilbiss Health Care, Inc. of Somerset, Pa.; see, e.g., the “Instruction Manual” for the “Ultra-Neb 99 Ultrasonic Nebulizer, publication A-850-C (published by DeVilbiss, Somerset, Pa., 1989).

In the embodiment shown in FIG. 2, the oscillators of ultrasonic nebulizer 13 are shown contacting an exterior surface of misting chamber 11. In this embodiment, the ultrasonic waves produced by the oscillators are transmitted via the walls of the misting chamber 11 and effect the misting of solution 9.

In another embodiment, not shown, the oscillators of ultrasonic nebulizer 13 are in direct contact with solution 9.

In one embodiment, it is preferred that the ultrasonic power used with such machine is in excess of one watt and, more preferably, in excess of 10 watts. In one embodiment, the power used with such machine exceeds about 50 watts.

During the time solution 9 is being caused to mist, it is preferably contacted with carrier gas to apply pressure to the solution and mist. It is preferred that a sufficient amount of carrier gas be introduced into the system at a sufficiently high flow rate so that pressure on the system is in excess of atmospheric pressure. Thus, for example, in one embodiment wherein chamber 11 has a volume of about 200 cubic centimeters, the flow rate of the carrier gas was from about 100 to about 150 milliliters per minute.

In one embodiment, the carrier gas 15 is introduced via feeding line 17 at a rate sufficient to cause solution 9 to mist at a rate of from about 0.5 to about 20 milliliters per minute. In one embodiment, the misting rate of solution 9 is from about 1.0 to about 3.0 milliliters per minute.

Substantially any gas that facilitates the formation of plasma may be used as carrier gas 15. Thus, by way of illustration, one may use oxygen, air, argon, nitrogen, and the like. It is preferred that the carrier gas used be a compressed gas under a pressure in excess 760 millimeters of mercury. In this embodiment, the use of the compressed gas facilitates the movement of the mist from the misting chamber 11 to the plasma region 21.

The misting container 11 may be any reaction chamber conventionally used by those skilled in the art and preferably is constructed out of such acid-resistant materials such as glass, plastic, and the like.

The mist from misting chamber 11 is fed via misting outlet line 19 into the plasma region 21 of plasma reactor 25. In plasma reactor 25, the mist is mixed with plasma generated by plasma gas 27 and subjected to radio frequency radiation provided by a radio-frequency coil 29.

The plasma reactor 25 provides energy to form plasma and to cause the plasma to react with the mist. Any of the plasmas reactors well known to those skilled in the art may be used as plasma reactor 25. Some of these plasma reactors are described in J. Mort et al.'s “Plasma Deposited Thin Films” (CRC Press Inc., Boca Raton, Fla., 1986); in “Methods of Experimental Physics,” Volume 9—Parts A and B, Plasma Physics (Academic Press, New York, 1970/1971); and in N. H. Burlingame's “Glow Discharge Nitriding of Oxides,” Ph.D. thesis (Alfred University, Alfred, N.Y., 1985), available from University Microfilm International, Ann Arbor, Mich.

In one preferred embodiment, the plasma reactor 25 is a “model 56 torch” available from the TAFA Inc. of Concord, N.H. It is preferably operated at a frequency of about 4 megahertz and an input power of 30 kilowatts.

Referring again to FIG. 2, and to the preferred embodiment depicted therein, it will be seen that into feeding lines 29 and 31 is fed plasma gas 27. As is known to those skilled in the art, a plasma can be produced by passing gas into a plasma reactor. A discussion of the formation of plasma is contained in B. Chapman's “Glow Discharge Processes” (John Wiley & Sons, New York, 1980)

In one preferred embodiment, the plasma gas used is a mixture of argon and oxygen. In another embodiment, the plasma gas is a mixture of nitrogen and oxygen. In yet another embodiment, the plasma gas is pure argon or pure nitrogen.

When the plasma gas is pure argon or pure nitrogen, it is preferred to introduce into the plasma reactor at a flow rate of from about 5 to about 30 liters per minute.

When a mixture of oxygen and either argon or nitrogen is used, the concentration of oxygen in the mixture preferably is from about 1 to about 40 volume percent and, more preferably, from about 15 to about 25 volume percent. When such a mixture is used, the flow rates of each gas in the mixture should be adjusted to obtain the desired gas concentrations. Thus, by way of illustration, in one embodiment that uses a mixture of argon and oxygen, the argon flow rate is 15 liters per minute, and the oxygen flow rate is 40 liters per minute.

In one embodiment, auxiliary oxygen 34 is fed into the top of reactor 25, between the plasma region 21 and the flame region 40, via lines 36 and 38. In this embodiment, the auxiliary oxygen is not involved in the formation of plasma but is involved in the enhancement of the oxidation of the ferrite material.

Radio frequency energy is applied to the reagents in the plasma reactor 25, and it causes vaporization of the mist.

In general, the energy is applied at a frequency of from about 100 to about 30,000 kilohertz. In one embodiment, the radio frequency used is from about 1 to 20 megahertz. In another embodiment, the radio frequency used is from about 3 to about 5 megahertz.

As is known to those skilled in the art, such radio frequency alternating currents may be produced by conventional radio frequency generators. Thus, by way of illustration, said TAPA Inc. “model 56 torch” may be attached to a radio frequency generator rated for operation at 35 kilowatts which manufactured by Lepel Company (a division of TAFA Inc.) and which generates an alternating current with a frequency of 4 megaherz at a power input of 30 kilowatts. Thus, e.g., one may use an induction coil driven at 2.5-5.0 megahertz that is sold as the “PLASMOC 2” by ENI Power Systems, Inc. of Rochester, N.Y.

The use of these type of radio-frequency generators is described in the Ph.D. theses entitled (1) “Heat Transfer Mechanisms in High-Temperature Plasma Processing of Glasses,” Donald M. McPherson (Alfred University, Alfred, N.Y., January, 1988) and (2) the aforementioned Nicholas H. Burlingame's “Glow Discharge Nitriding of Oxides.”

The plasma vapor 23 formed in plasma reactor 25 is allowed to exit via the aperture 42 and can be visualized in the flame region 40. In this region, the plasma contacts air that is at a lower temperature than the plasma region 21, and a flame is visible. A theoretical model of the plasma/flame is presented on pages 88 et seq. of said McPherson thesis.

The vapor 44 present in flame region 40 is propelled upward towards substrate 46. Any material onto which vapor 44 will condense may be used as a substrate. Thus, by way of illustration, one may use nonmagnetic materials such alumina, glass, gold-plated ceramic materials, and the like. In one embodiment, substrate 46 consists essentially of a magnesium oxide material such as single crystal magnesium oxide, polycrystalline magnesium oxide, and the like.

In another embodiment, the substrate 46 consists essentially of zirconia such as, e.g:, yttrium stabilized cubic zirconia.

In another embodiment, the substrate 46 consists essentially of a material selected from the group consisting of strontium titanate, stainless steel, alumina, sapphire, and the like.

The aforementioned listing of substrates is merely meant to be illustrative, and it will be apparent that many other substrates may be used. Thus, by way of illustration, one may use any of the substrates mentioned in M. Sayer's “Ceramic Thin Films . . . ” article, supra. Thus, for example, in one embodiment it is preferred to use one or more of the substrates described on page 286 of “Superconducting Devices,” edited by S. T. Ruggiero et al. (Academic Press, Inc., Boston, 1990).

One advantage of this embodiment of applicants' process is that the substrate may be of substantially any size or shape, and it may be stationary or movable. Because of the speed of the coating process, the substrate 46 may be moved across the aperture 42 and have any or all of its surface be coated.

As will be apparent to those skilled in the art, in the embodiment depicted in FIG. 2, the substrate 46 and the coating 48 are not drawn to scale but have been enlarged to the sake of ease of representation.

Referring again to FIG. 2, the substrate 46 may be at ambient temperature. Alternatively, one may use additional heating means to heat the substrate prior to, during, or after deposition of the coating.

In one embodiment, illustrated in FIG. 2A, the substrate is cooled so that nanomagnetic particles are collected on such substrate. Referring to FIG. 2A, and in the preferred embodiment depicted therein, a precursor 1 that preferably contains moieties A, B, and C (which are described elsewhere in this specification) are charged to reactor 3; the reactor 3 may be the plasma reactor depicted in FIG. 2, and/or it may be the sputtering reactor described elsewhere in this specification.

Referring again to FIG. 2A, it will be seen that an energy source 5 is preferably used in order to cause reaction between moieties A, B, and C. The energy source 5 may be an electromagnetic energy source that supplies energy to the reactor 3. In one embodiment, there are at least two species of moiety A present, and at least two species of moiety C present. The two preferred moiety C species are oxygen and nitrogen.

Within reactor 3 moieties A, B, and C are preferably combined into a metastable state. This metastable state is then caused to travel towards collector 7. Prior to the time it reaches the collector 7, the ABC moiety is formed, either in the reactor 3 and/or between the reactor 3 and the collector 7.

In one embodiment, collector 7 is preferably cooled with a chiller 99 so that its surface 111 is at a temperature below the temperature at which the ABC moiety interacts with surface 111; the goal is to prevent bonding between the ABC moiety and the surface 111. In one embodiment, the surface 111 is at a temperature of less than about 30 degrees Celsius. In another embodiment, the temperature of surface 111 is at the liquid nitrogen temperature, i.e., about 77 degrees Kelvin.

After the ABC moieties have been collected by collector 7, they are removed from surface 111.

Referring again to FIG. 2, and in one preferred embodiment, a heater (not shown) is used to heat the substrate to a temperature of from about 100 to about 800 degrees centigrade.

In one aspect of this embodiment, temperature sensing means (not shown) may be used to sense the temperature of the substrate and, by feedback means (not shown), adjust the output of the heater (not shown). In one embodiment, not shown, when the substrate 46 is relatively near flame region 40, optical pyrometry measurement means (not shown) may be used to measure the temperature near the substrate.

In one embodiment, a shutter (not shown) is used to selectively interrupt the flow of vapor 44 to substrate 46. This shutter, when used, should be used prior to the time the flame region has become stable; and the vapor should preferably not be allowed to impinge upon the substrate prior to such time.

The substrate 46 may be moved in a plane that is substantially parallel to the top of plasma chamber 25. Alternatively, or additionally, it may be moved in a plane that is substantially perpendicular to the top of plasma chamber 25. In one embodiment, the substrate 46 is moved stepwise along a predetermined path to coat the substrate only at certain predetermined areas.

In one embodiment, rotary substrate motion is utilized to expose as much of the surface of a complex-shaped article to the coating. This rotary substrate motion may be effectuated by conventional means. See, e.g., “Physical Vapor Deposition,” edited by Russell J. Hill (Temescal Division of The BOC Group, Inc., Berkeley, Calif., 1986).

The process of this embodiment of the invention allows one to coat an article at a deposition rate of from about 0.01 to about 10 microns per minute and, preferably, from about 0.1 to about 1.0 microns per minute, with a substrate with an exposed surface of 35 square centimeters. One may determine the thickness of the film coated upon said reference substrate material (with an exposed surface of 35 square centimeters) by means well known to those skilled in the art.

The film thickness can be monitored in situ, while the vapor is being deposited onto the substrate. Thus, by way of illustration, one may use an IC-6000 thin film thickness monitor (also referred to as “deposition controller”) manufactured by Leybold Inficon Inc. of East Syracuse, N.Y.

The deposit formed on the substrate may be measured after the deposition by standard profilometry techniques. Thus, e.g., one may use a DEKTAK Surface Profiler, model number 900051 (available from Sloan Technology Corporation, Santa Barbara, California).

In general, at least about 80 volume percent of the particles in the as-deposited film are smaller than about 1 micron. It is preferred that at least about 90 percent of such particles are smaller than 1 micron. Because of this fine grain size, the surface of the film is relatively smooth.

In one preferred embodiment, the as-deposited film is post-annealed.

It is preferred that the generation of the vapor in plasma rector 25 be conducted under substantially atmospheric pressure conditions. As used in this specification, the term “substantially atmospheric” refers to a pressure of at least about 600 millimeters of mercury and, preferably, from about 600 to about 1,000 millimeters of mercury. It is preferred that the vapor generation occur at about atmospheric pressure. As is well known to those skilled in the art, atmospheric pressure at sea level is 760 millimeters of mercury.

The process of this invention may be used to produce coatings on a flexible substrate such as, e.g., stainless steel strips, silver strips, gold strips, copper strips, aluminum strips, and the like. One may deposit the coating directly onto such a strip. Alternatively, one may first deposit one or more buffer layers onto the strip(s). In other embodiments, the process of this invention may be used to produce coatings on a rigid or flexible cylindrical substrate, such as a tube, a rod, or a sleeve.

Referring again to FIG. 2, and in the embodiment depicted therein, as the coating 48 is being deposited onto the substrate 46, and as it is undergoing solidification thereon, it is preferably subjected to a magnetic field produced by magnetic field generator 50.

In this embodiment, it is preferred that the magnetic field produced by the magnetic field generator 50 have a field strength of from about 2 Gauss to about 40 Tesla.

It is preferred to expose the deposited material for at least 10 seconds and, more preferably, for at least 30 seconds, to the magnetic field, until the magnetic moments of the nano-sized particles being deposited have been substantially aligned.

As used herein, the term “substantially aligned” means that the inductance of the device being formed by the deposited nano-sized particles is at least 90 percent of its maximum inductance. One may determine when such particles have been aligned by, e.g., measuring the inductance, the permeability, and/or the hysteresis loop of the deposited material.

Thus, e.g., one may measure the degree of alignment of the deposited particles with an impedance meter, a inductance meter, or a SQUID.

In one embodiment, the degree of alignment of the deposited particles is measured with an inductance meter. One may use, e.g., a conventional conductance meter such as, e.g., the conductance meters disclosed in U.S. Pat. Nos. 4,779,462, 4,937,995, 5,728,814 (apparatus for determining and recording injection does in syringes using electrical inductance), 6,318,176, 5,014,012, 4,869,598, 4,258,315 (inductance meter), 4,045,728 (direct reading inductance meter), 6,252,923, 6,194,898, 6,006,023 (molecular sensing apparatus), 6,048,692 (sensors for electrically sensing binding events for supported molecular receptors), and the like. The entire disclosure of each of these United States patents is hereby incorporated by reference into this specification.

When measuring the inductance of the coated sample, the inductance is preferably measured using an applied wave with a specified frequency. As the magnetic moments of the coated samples align, the inductance increases until a specified value; and it rises in accordance with a specified time constant in the measurement circuitry.

In one embodiment, the deposited material is contacted with the magnetic field until the inductance of the deposited material is at least about 90 percent of its maximum value under the measurement circuitry. At this time, the magnetic particles in the deposited material have been aligned to at least about 90 percent of the maximum extent possible for maximizing the inductance of the sample.

By way of illustration and not limitation, a metal rod with a diameter of 1 micron and a length of 1 millimeter, when uncoated with magnetic nano-sized particles, might have an inductance of about 1 nanohenry. When this metal rod is coated with, e.g., nano-sized ferrites, then the inductance of the coated rod might be 5 nanohenries or more. When the magnetic moments of the coating are aligned, then the inductance might increase to 50 nanohenries, or more. As will be apparent to those skilled in the art, the inductance of the coated article will vary, e.g., with the shape of the article and also with the frequency of the applied electromagnetic field.

One may use any of the conventional magnetic field generators known to those skilled in the art to produce such as magnetic field. Thus, e.g., one may use one or more of the magnetic field generators disclosed in U.S. Pat. Nos. 6,503,364, 6,377,149 (magnetic field generator for magnetron plasma generation), 6,353,375 (magnetostatic wave device), 6,340,888 (magnetic field generator for MRI), 6,336,989, 6,335,617 (device for calibrating a magnetic field generator), 6,313,632, 6,297,634, 6,275,128, 6,246,066 (magnetic field generator and charged particle beam irradiator), 6,114,929 (magnetostatic wave device), 6,099,459 (magnetic field generating device and method of generating and applying a magnetic field), 5,795,212, 6,106,380 (deterministic magnetorheological finishing), 5,839,944 (apparatus for deterministic magnetorheological finishing), 5,971,835 (system for abrasive jet shaping and polishing of a surface using a magnetorheological fluid), 5,951,369, 6,506,102 (system for magnetorheological finishing of substrates), 6,267,651, 6,309,285 (magnetic wiper), and the like. The entire disclosure of each of these United States patents is hereby incorporated by reference into this specification.

In one embodiment, the magnetic field is 1.8 Tesla or less. In this embodiment, the magnetic field can be applied with, e.g., electromagnets disposed around a coated substrate.

For fields greater than about 2 Tesla, one may use superconducting magnets that produce fields as high as 40 Tesla. Reference may be had, e.g., to U.S. Pat. Nos. 5,319,333 (superconducting homogeneous high field magnetic coil), 4,689,563, 6,496,091 (superconducting magnet arrangement), 6,140,900 (asymmetric superconducting magnets for magnetic resonance imaging), 6,476,700 (superconducting magnet system), 4,763,404 (low current superconducting magnet), 6,172,587 (superconducting high field magnet), 5,406,204, and the like. The entire disclosure of each of these United States patents is hereby incorporated by reference into this specification.

In one embodiment, no magnetic field is applied to the deposited coating while it is being solidified. In this embodiment, as will be apparent to those skilled in the art, there still may be some alignment of the magnetic domains in a plane parallel to the surface of substrate as the deposited particles are locked into place in a matrix (binder) deposited onto the surface.

In one embodiment, depicted in FIG. 2, the magnetic field 52 is preferably delivered to the coating 48 in a direction that is substantially parallel to the surface 56 of the substrate 46. In another embodiment, depicted in FIG. 1, the magnetic field 58 is delivered in a direction that is substantially perpendicular to the surface 56. In yet another embodiment, the magnetic field 60 is delivered in a direction that is angularly disposed vis-a-vis surface 56 and may form, e.g., an obtuse angle (as in the case of field 62). As will be apparent, combinations of these magnetic fields may be used.

FIG. 3 is a flow diagram of another process that may be used to make the nanomagnetic compositions of this invention. Referring to FIG. 3, and to the preferred process depicted therein, it will be seen that nano-sized ferromagnetic material(s), with a particle size less than about 100 nanometers, is preferably charged via line 60 to mixer 62. It is preferred to charge a sufficient amount of such nano-sized material(s) so that at least about 10 weight percent of the mixture formed in mixer 62 is comprised of such nano-sized material. In one embodiment, at least about 40 weight percent of such mixture in mixer 62 is comprised of such nano-sized material. In another embodiment, at least about 50 weight percent of such mixture in mixer 62 is comprised of such nano-sized material.

In one embodiment, one or more binder materials are charged via line 64 to mixer 62. In one embodiment, the binder used is a ceramic binder. These ceramic binders are well known. Reference may be had, e.g., to pages 172-197 of James S. Reed's “Principles of Ceramic Processing,” Second Edition (John Wiley & Sons, Inc., New York, N.Y., 1995). As is disclosed in the Reed book, the binder may be a clay binder (such as fine kaolin, ball clay, and bentonite), an organic colloidal particle binder (such as microcrystalline cellulose), a molecular organic binder (such as natural gums, polyscaccharides, lignin extracts, refined alginate, cellulose ethers, polyvinyl alcohol, polyvinylbutyral, polymethyl methacrylate, polyethylene glycol, paraffin, and the like.). etc.

In one embodiment, the binder is a synthetic polymeric or inorganic composition. Thus, and referring to George S. Brady et al.'s “Materials Handbook,” (McGraw-Hill, Inc., New York, N.Y. 1991), the binder may be acrylonitrile-butadiene-styrene (see pages 5-6), an acetal resin (see pages 6-7), an acrylic resin (see pages 10-12), an adhesive composition (see pages 14-18), an alkyd resin (see page 27-28), an allyl plastic (see pages 31-32), an amorphous metal (see pages 53-54), a biocompatible material (see pages 95-98), boron carbide (see page 106), boron nitride (see page 107), camphor (see page 135), one or more carbohydrates (see pages 138-140), carbon steel (see pages 146-151), casein plastic (see page 157), cast iron (see pages 159-164), cast steel (see pages 166-168), cellulose (see pages 172-175), cellulose acetate (see pages 175-177), cellulose nitrate (see pages 177), cement (see page 178-180), ceramics (see pages 180-182), cermets (see pages 182-184), chlorinated polyethers (see pages 191-191), chlorinated rubber (see pages 191-193), cold-molded plastics (see pages 220-221), concrete (see pages 225-227), conductive polymers and elastomers (see pages 227-228), degradable plastics (see pages 261-262), dispersion-strengthened metals (see pages 273-274), elastomers (see pages 284-290), enamel (see pages 299-301), epoxy resins (see pages 301-302), expansive metal (see page 313), ferrosilicon (see page 327), fiber-reinforced plastics (see pages 334-335), fluoroplastics (see pages 345-347), foam materials (see pages 349-351), fusible alloys (see pages 362-364), glass (see pages 376-383), glass-ceramic materials (see pages 383-384), gypsum (see pages 406-407), impregnated wood (see pages 422-423), latex (see pages 456-457), liquid crystals (see page 479). lubricating grease (see pages 488-492), magnetic materials (see pages 505-509), melamine resin (see pages 5210-521), metallic materials (see pages 522-524), nylon (see pages 567-569), olefin copolymers (see pages 574-576), phenol-formaldehyde resin (see pages 615-617), plastics (see pages 637-639), polyarylates (see pages 647-648), polycarbonate resins (see pages 648), polyester thermoplastic resins (see pages 648-650), polyester thermosetting resins (see pages 650-651), polyethylenes (see pages 651-654), polyphenylene oxide (see pages 644-655), polypropylene plastics (see pages 655-656), polystyrenes (see pages 656-658), proteins (see pages 666-670), refractories (see pages 691-697), resins (see pages 697-698), rubber (see pages 706-708), silicones (see pages 747-749), starch (see pages 797-802), superalloys (see pages 819-822), superpolymers (see pages 823-825), thermoplastic elastomers (see pages 837-839), urethanes (see pages 874-875), vinyl resins (see pages 885-888), wood (see pages 912-916), mixtures thereof, and the like.

Referring again to FIG. 3, one may charge to line 64 either one or more of these “binder material(s)” and/or the precursor(s) of these materials that, when subjected to the appropriate conditions in former 66, will form the desired mixture of nanomagnetic material and binder.

Referring again to FIG. 3, and in the preferred process depicted therein, the mixture within mixer 62 is preferably stirred until a substantially homogeneous mixture is formed. Thereafter, it may be discharged via line 65 to former 66.

One process for making a fluid composition comprising nanomagnetic particles is disclosed in U.S. Pat. No. 5,804,095, “Magnetorheological Fluid Composition,”, of Jacobs et al; the disclosure of this patent is incorporated herein by reference. In this patent, there is disclosed a process comprising numerous material handling steps used to prepare a nanomagnetic fluid comprising iron carbonyl particles. One suitable source of iron carbonyl particles having a median particle size of 3.1 microns is the GAF Corporation.

The process of Jacobs et al, is applicable to the present invention, wherein such nanomagnetic fluid further comprises a polymer binder, thereby forming a nanomagnetic paint. In one embodiment, the nanomagnetic paint is formulated without abrasive particles of cerium dioxide. In another embodiment, the nanomagnetic fluid further comprises a polymer binder, and aluminum nitride is substituted for cerium dioxide.

There are many suitable mixing processes and apparatus for the milling, particle size reduction, and mixing of fluids comprising solid particles. For example, e.g., iron carbonyl particles or other ferromagnetic particles of the paint may be further reduced to a size on the order of 100 nanometers or less, and/or thoroughly mixed with a binder polymer and/or a liquid solvent by the use of a ball mill, a sand mill, a paint shaker holding a vessel containing the paint components and hard steel or ceramic beads; a homogenizer (such as the Model Ytron Z made by the Ytron Quadro Corporation of Chesham, United Kingdom, or the Microfluidics M700 made by the MFIC Corporation of Newton, Mass.), a powder dispersing mixer (such as the Ytron Zyclon mixer, or the Ytron Xyclon mixer, or the Ytron PID mixer by the Ytron Quadro Corporation); a grinding mill (such as the Model F10 Mill by the Ytron Quadro Corporation); high shear mixers (such as the Ytron Y mixer by the Ytron Quadro Corporation), the Silverson Laboratory Mixer sold by the Silverson Corporation of East Longmeadow, Mass., and the like. The use of one or more of these apparatus in series or in parallel may produce a suitably formulated nanomagnetic paint.

Referring again to FIG. 3, the former 66 is preferably equipped with an input line 68 and an exhaust line 70 so that the atmosphere within the former can be controlled. One may utilize an ambient atmosphere, an inert atmosphere, pure nitrogen, pure oxygen, mixtures of various gases, and the like. Alternatively, or additionally, one may use lines 68 and 70 to afford subatmospheric pressure, atmospheric pressure, or superatomspheric pressure within former 66.

In the embodiment depicted, former 66 is also preferably comprised of an electromagnetic coil 72 that, in response from signals from controller 74, can control the extent to which, if any, a magnetic field is applied to the mixture within the former 66 (and also within the mold 67 and/or the spinnerette 69).

The controller 74 is also adapted to control the temperature within the former 66 by means of heating/cooling assembly.

In the embodiment depicted in FIG. 3, a sensor 78 preferably determines the extent to which the desired nanomagnetic properties have been formed with the nano-sized material in the former 66; and, as appropriate, the sensor 78 imposes a magnetic field upon the mixture within the former 66 until the desired properties have been obtained.

In one embodiment, the sensor 78 is the inductance meter discussed elsewhere in this specification; and the magnetic field is applied until at least about 90 percent of the maximum inductance obtainable with the alignment of the magnetic moments has been obtained.

The magnetic field is preferably imposed until the nano-sized particles within former 78 (and the material with which it is admixed) have a mass density of at least about 0.001 grams per cubic centimeter (and preferably at least about 0.01 grams per cubic centimeter), a saturation magnetization of from about 1 to about 36,000 Gauss, a coercive force of from about 0.01 to about 5,000 Oersteds, and a relative magnetic permeability of from about 1 to about 500,000.

When the mixture within former 66 has the desired combination of properties (as reflected, e.g., by its substantially maximum inductance) and/or prior to that time, some or all of such mixture may be discharged via line 80 to a mold/extruder 67 wherein the mixture can be molded or extruded into a desired shape. A magnetic coil 72 also preferably may be used in mold/extruder 67 to help align the nano-sized particles.

Alternatively, or additionally, some or all of the mixture within former 66 may be discharged via line 82 to a spinnerette 69, wherein it may be formed into a fiber (not shown).

As will be apparent, one may make fibers by the process indicated that have properties analogous to the nanomagnetic properties of the coating 135 (described elsewhere in this specification), and/or nanoelectrical properties of the coating 141 (described elsewhere in this specification), and/or nanothermal properties of the coating 145 (also described elsewhere in this specification). Such fiber or fibers may be made into fabric by conventional means. By the appropriate selection and placement of such fibers, one may produce a shielded fabric which provides protection against high magnetic voltages and/or high voltages and/or excessive heat. Such shielded fabric may comprise the polymeric material 14 (see FIG. 1).

Thus, in one embodiment, nanomagnetic and/or nanoelectrical and/or nanothermal fibers are woven together to produce a garment that will shield from the adverse effects of radiation such as, e.g., radiation experienced by astronauts in outer space. Such fibers may comprise the polymeric material 14 (see FIG. 1).

Alternatively, or additionally, some or all of the mixture within former 66 may be discharged via line 84 to a direct writing applicator 90, such as a MicroPen applicator manufactured by OhmCraft Incorporated of Honeoye Falls, N.Y. Such an applicator is disclosed in U.S. Pat. No. 4,485,387, the disclosure of which is incorporated herein by reference. The use of this applicator to write circuits and other electrical structures is described in, e.g., U.S. Pat. No. 5,861,558 of Buhl et al, “Strain Gauge and Method of Manufacture”, the disclosure of which is incorporated herein by reference.

In one preferred embodiment, the nanomagnetic, nanoelectrical, and/or nanothermal compositions of the present invention, along with various conductor, resistor, capacitor, and inductor formulations, are dispensed by the MicroPen device, to fabricate the circuits and structures of the present invention on devices such as, e.g. catheters and other biomedical devices.

In one preferred embodiment, involving the writing of nanomagnetic circuit patterns and/or thin films, the direct writing applicator 90 (as disclosed in U.S. Pat. No. 4,485,387) comprises an applicator tip 92 and an annular magnet 94, which provides a magnetic field 72. The use of such an applicator 90 to apply nanomagnetic coatings is particularly beneficial because the presence of the magnetic field from magnet 94, through which the nanomagnetic fluid flows serves to orient the magnetic particles in situ as such nanomagnetic fluid is applied to a substrate. Such an orienting effect is described in U.S. Pat. No. 5,971,835, the disclosure of which is incorporated herein by reference. Once the nanomagnetic particles are properly oriented by such a field, or by another magnetic field source, the applied coating is cured by heating, by ultraviolet radiation, by an electron beam, or by other suitable means.

In one embodiment, not shown, one may form compositions comprised of nanomagentic particles and/or nanoelectrical particles and/or nanothermal particles and/or other nano-sized particles by a sol-gel process. Thus, by way of illustration and not limitation, one may use one or more of the processes described in U.S. Pat. Nos. 6,287,639 (nanocomposite material comprised of inorganic particles and silanes), 6,337,117 (optical memory device comprised of nano-sized luminous material), 6,527,972 (magnetorheological polymer gels), 6,589,457 (process for the deposition of ruthenium oxide thin films), 6,657,001 (polysiloxane compositions comprised of inorganic particles smaller than 100 nanometers), 6,666,935 (sol-gel manufactured energetic materials), and the like. The entire disclosure of each of these United States patents is hereby incorporated by reference into this specification.

Nanomagnetic Compositions Comprised of Moieties A, B, and C

The aforementioned process described in the preceding section of this specification, and the other processes described in this specification, may each be adapted to produce other, comparable nanomagnetic structures, as is illustrated in FIG. 4.

Referring to FIG. 4, and in the preferred embodiment depicted therein, a phase diagram 100 is presented. As is illustrated by this phase diagram 100, the nanomagnetic material used in this embodiment of the invention preferably is comprised of one or more of moieties A, B, and C. The moieties A, B, and C described in reference to phase 100 of FIG. 4 are not necessarily the same as the moieties A, B, and C described in reference to phase diagram 2000 described elsewhere in this specification.

In the embodiment depicted, the moiety A depicted in phase diagram 100 is preferably comprised of a magnetic element selected from the group consisting of a transition series metal, a rare earth series metal, or actinide metal, a mixture thereof, and/or an alloy thereof. In one embodiment, the moiety A is iron. In another embodiment, moiety A is nickel. In yet another embodiment, moiety A is cobalt. In yet another embodiment, moiety A is gadolinium. In another embodiment, the A moiety is selected from the group consisting of samarium, holmium, neodymium, and one or more other members of the Lanthanide series of the periodic table of elements.

In one preferred embodiment, two or more A moieties are present, as atoms. In one aspect of this embodiment, the magnetic susceptibilities of the atoms so present are both positive.

In one embodiment, two or more A moieties are present, at least one of which is iron. In one aspect of this embodiment, both iron and cobalt atoms are present.

When both iron and cobalt are present, it is preferred that from about 10 to about 90 mole percent of iron be present by mole percent of total moles of iron and cobalt present in the ABC moiety. In another embodiment, from about 50 to about 90 mole percent of iron is present. In yet another embodiment, from about 60 to about 90 mole percent of iron is present. In yet another embodiment, from about 70 to about 90 mole percent of iron is present.

As is known to those skilled in the art, the transition series metals include chromium, manganese, iron, cobalt, and nickel. One may use alloys of iron, cobalt and nickel such as, e.g., iron-aluminum, iron-carbon, iron-nchromium, iron-cobalt, iron-nickel, iron nitride (Fe₃N), iron phosphide, iron-silicon, iron-vanadium, nickel-cobalt, nickel-copper, and the like. One may use alloys of manganese such as, e.g., manganese-aluminum, manganese-bismuth, MnAs, MnSb, MnTe, manganese-copper, manganese-gold, manganese-nickel, manganese-sulfur and related compounds, manganese-antimony, manganese-tin, manganese-zinc, Heusler alloy W, and the like. One may use compounds and alloys of the iron group, including oxides of the iron group, halides of the iron group, borides of the transition elements, sulfides of the iron group, platinum and palladium with the iron group, chromium compounds, and the like.

One may use a rare earth and/or actinide metal such as, e.g., Ce, Pr, Nd, Pm, Sm, Eu, Gd, Tb, Dy, Ho, Er, Tm, Yb, Lu, La, mixtures thereof, and alloys thereof. One may also use one or more of the actinides such as, e.g., the actinides of Th, Pa, U, Np, Pu, Am, Cm, Bk, Cf, Es, Fm, Md, No, Lr, Ac, and the like.

These moieties, compounds thereof, and alloys thereof are well known and are described, e.g., in the text of R. S. Tebble et al. entitled “Magnetic Materials.”

In one preferred embodiment, illustrated in FIG. 4, moiety A is selected from the group consisting of iron, nickel, cobalt, alloys thereof, and mixtures thereof. In this embodiment, the moiety A is magnetic, i.e., it has a relative magnetic permeability of from about 1 to about 500,000. As is known to those skilled in the art, relative magnetic permeability is a factor, being a characteristic of a material, which is proportional to the magnetic induction produced in a material divided by the magnetic field strength; it is a tensor when these quantities are not parallel. See, e.g., page 4-128 of E. U. Condon et al.'s “Handbook of Physics” (McGraw-Hill Book Company, Inc., New York, N.Y., 1958).

The moiety A of FIG. 4 also preferably has a saturation magnetization of from about 1 to about 36,000 Gauss, and a coercive force of from about 0.01 to about 5,000 Oersteds.

The moiety A of FIG. 4 may be present in the nanomagnetic material either in its elemental form, as an alloy, in a solid solution, or as a compound.

It is preferred at least about 1 mole percent of moiety A be present in the nanomagnetic material (by total moles of A, B, and C), and it is more preferred that at least 10 mole percent of such moiety A be present in the nanomagnetic material (by total moles of A, B, and C). In one embodiment, at least 60 mole percent of such moiety A is present in the nanomagnetic material, (by total moles of A, B, and C.)

In one embodiment, the nanomagnetic material has the formula A₁A₂(B)ₓC₁(C₂)_{y}, wherein each of A₁ and A₂ are separate magnetic A moieties, as described above; B is as defined elsewhere in this specification; x is an integer from 0 to 1; each of C₁ and C₂ is as descried elsewhere in this specification; and y is an integer from 0 to 1.

In this embodiment, there are always two distinct A moieties, such as, e.g., nickel and iron, iron and cobalt, etc. The A moieties may be present in equimolar amounts; or they may be present in non-equimolar amount.

In one aspect of this embodiment, either or both of the A₁ and A₂ moieties are radioactive. Thus, e.g., either or both of the A₁ and A₂ moieties may be selected from the group consisting of radioactive cobalt, radioactive iron, radioactive nickel, and the like. These radioactive isotopes are well known. Reference may be had, e.g., to U.S. Pat. Nos. 3,894,584; 3,936,440 (method of labeling coplex metal chelates with radioactive metal isotopes); 4,031,387; 4,282,092; 4,572,797; 4,642,193; 4,659,512; 4,704,245; 4,758,874 (minimization of radioactive material deposition in water-cooled nuclar reactors); 4,950,449 (inhibition of radioactive cobalt deposition); 4,647,585 (method for separating cobalt, nickel, and the like from alloys), 4,759,900; 4,781,198 (biopsy tracer needle); 4,876,449; 5,035,858; 5,196,113; 5,205,167; 5,222,065; 5,241,060 (base moiety-labeled detectable nucleotide); 6,314,153; and the like. The entire disclosure of each of these United States patents is herey incorporated by reference into this specification.

In one preferred embodiment, at least one of the A₁ and A₂ moieties is radioactive cobalt. This radioisotope is discussed, e.g., in U.S. Pat. No. 3,936,440, the entire disclosure of which is hereby incorporated by reference into this specification. As is disclosed in this patent, “Complex metal chelate compounds containing radioactive metal isotopes have been known and utilized in the prior art. For example, “tagged” Vitamin B12, that is Vitamin B12 containing a radioactive isotope of cobalt, has been used in the diagnosis of pernicious anemia and has been prepared via biochemical synthesis, wherein microbes are cultured in the presence of a cobalt-57 salt and produce Vitamin B12 containing cobalt-57 isotopes which must then be purified by lengthy chromotographic separations . . . . In accordance with the present invention, a method is provided for labeling a complex metal chelate with a radioactive metal isotope via isotopic exchange in the solid state between the metal atom of the complex metal chelate and the radioactive metal isotope . . . . In accordance with the present invention, any metal chelate compound, including cyanocobalamin, cobaltocene, aquocobalamin, porphyrins, phthalocyanines and other macrocyclic compounds, may be labeled with a radioactive isotope of either the same metal as that present in the complex metal chelate compound or a different metal than that present in the complex metal chelate compound . . . . Typical of the radioactive metal isotopes which are within the purview of the present invention are 57 Co+2, 60 Co+2, 52 Fe+2, 52 Fe+3, 48 Cr+3, 95 Tc+4, 97 Tc+4 and 99 Tc+4 . . . .”

As is also disclosed in U.S. Pat. No. 3,936,440, “In accordance with the present invention, one preferred embodiment provides a method for labeling Vitamin B12, that is cyanocobalamin, with 57 Co+2, a radioactive isotope of cobalt. It is to be understood, however, that it is fully within the purview of the present invention to substitute other radioactive isotopes of cobalt, such as 60 Co+2, or radioactive isotopes of other metals within the scope of the present invention.”

In one embodiment, at least one of the A₁ and A₂ is radioactive iron. This radioisotope is also well known as is evidenced, e.g., by U.S. Pat. No. 4,459,356, the entire disclosure of which is also hereby incorporated by reference into this specification. Thus, and as is disclosed in such patent, “In accordance with the present invention, a radioactive stain composition is developed as a result of introduction of a radionuclide (e.g., radioactive iron isotope 59 Fe, which is a strong gamma emitter having peaks of 1.1 and 1.3 MeV) into BPS to form ferrous BPS . . . . In order to prepare the radioactive stain composition, sodium bathophenanthroline sulfonate (BPS), ascorbic acid and Tris buffer salts were obtained from Sigma Chemical Co. (St. Louis, MO). Enzymes grade acrylamide, N,N′ methylenebisacrylamide and N,N,N′,N′-tetramethylethylenediamine (TEMED) are products of and were obtained from Eastman Kodak Co. (Rochester, N.Y.). Sodium dodecylsulfate (SDS) was obtained from Pierce Chemicals (Rockford, Ill.). The radioactive isotope (59 FeCl3 in 0.05M HCl, specific activity 15.6 mC/mg) was purchased from New England Nuclear (Boston, Mass.), but was diluted to 10 ml with 0.5N HCl to yield an approximately 0.1 mM Fe(III) solution.”

Referring again to FIG. 4, and to the preferred embodiment depicted therein, in this embodiment, there may be, but need not be, a B moiety (such as, e.g., aluminum). There preferably are at least two C moieties such as, e.g., oxygen and nitrogen. The A moieties, in combination, comprise at least about 80 mole percent of such a composition; and they preferably comprise at least 90 mole percent of such composition.

When two C moieties are present, and when the two C moieties are oxygen and nitrogen,they preferably are present in a mole ratio such that from about 10 to about 90 mole percent of oxygen is present, by total moles of oxygen and nitrogen. It is preferred that at least about 60 mole percent of oxygen be present. In one embodiment, at least about 70 mole percent of oxygen is so present. In yet another embodiment, at least 80 mole percent of oxygen is so present.

One may measure the surface of the nanomagnetic material, measuring the first 8.5 nanometers of material. When such surface is measured, it is preferred that at least 50 mole percent of oxygen, by total moles of oxygen and nitrogen, be present in such surface. It is preferred that at least about 60 mole percent of oxygen be present. In one embodiment, at least about 70 mole percent of oxygen is so present. In yet another embodiment, at least 80 mole percent of oxygen is so present.

Without wishing to be bound to any particular theory, applicants believe that the presence of two distinct A moieties in their compositon, and two distinct C moieties (such as, e.g., oxygen and nitrogen), provides better magnetic properties for applicants' nanomagmetic materials.

In the embodiment depicted in FIG. 4, in addition to moiety A, it is preferred to have moiety B be present in the nanomagnetic material. In this embodiment, moieties A and B are admixed with each other. The mixture may be a physical mixture, it may be a solid solution, it may be comprised of an alloy of the A/B moieties, etc.

The Squareness of the Nanomagnetic Particles of the Invention

As is known to those skilled in the art, the squareness of a magnetic material is the ratio of the residual magnetic flux and the saturation magnetic flux density. Reference may be had, e.g., to U.S. Pat. Nos. 6,627,313, 6,517,934, 6,458,452, 6,391,450, 6,350,505, 6,248,437, 6,194,058, 6,042,937, 5,998,048, 5,645,652, and the like. The entire disclosure of such United States patents is hereby incorporated by reference into this specification. Reference may also be had to page 1802 of the McGraw-Hill Dictionary of Scientific and Techical Terms, Fourth Edition (McGraw-Hill Book Company, New York, N.Y., 1989). At such page 1802, the “squareness ratio” is defined as “The magnetic induction at zero magnetizing force divided by the maximum magnetic indication, in a symmetric cyclic magnetization of a material.”

In one embodiment, the squareness of applicants' nanomagnetic material 32 is from about 0.05 to about 1.0. In one aspect of this embodiment, such squareness is from about 0.1 to about 0.9. In another aspect of this embodiment, the squareness is from about 0.2 to about 0.8. In applications where a large residual magnetic moment is desired, the squareness is preferably at least about 0.8.

Referring again to FIG. 4, and in the preferred embodiment depicted therein, the nanomagnetic material may be comprised of 100 percent of moiety A, provided that such moiety A has the required normalized magnetic interaction (M). Alternatively, the nanomagnetic material may be comprised of both moiety A and moiety B. In one embodiment, the A moieties comprise at least about 80 mole percent (and preferably at least about 90 mole percent) of the total moles of the A, B, and C moieties.

When moiety B is present in the nanomagnetic material, in whatever form or forms it is present, it is preferred that it be present at a mole ratio (by total moles of A and B) of from about 1 to about 99 percent and, preferably, from about 10 to about 90 percent.

The B moiety, in one ebodiment, in whatever form it is present, is preferably nonmagnetic, i.e., it has a relative magnetic permeability of about 1.0; without wishing to be bound to any particular theory, applicants believe that the B moiety acts as buffer between adjacent A moieties. One may use, e.g., such elements as silicon, aluminum, boron, platinum, tantalum, palladium, yttrium, zirconium, titanium, calcium, beryllium, barium, silver, gold, indium, lead, tin, antimony, germanium, gallium, tungsten, bismuth, strontium, magnesium, zinc, and the like.

In one embodiment, the B moiety has a relative magnetic permeability that is about equal to 1 plus the magnetic susceptilibity. The relative magnetic susceptilities of silicon, aluminum, boron, platinum, tantalum, palladium, yttrium, zirconium, titanium, calcium, beryllium, barium, silver, gold, indium, lead, tin, antimony, germanium, gallium, tungsten, bismuth, strontium, magnesium, zinc, copper, cesium, cerium, hafnium, iodine, iridium, lanthanum, lithium, lutetium, manganese, molybdenum, potassium, sodium, strontium, praseodymium, rhenium, rhodium, rubidium, ruthenium, scandium, selenium, tantalum, technetium, tellurium, chromium, thallium, thorium, thulium, titanium, vanadium, zinc, yttrium, ytterbium, zirconium, and the like. Reference may be had, e.g., to pages E-118 through E 123 of the aforementioned CRC Handbook of Chemistry and Physics.

In one embodiment, the nanomagnetic particles may be represented by the formula AₓB_{y}C_{z} wherein x+y+z is equal to 1. In this embodiment the ratio of x/y is at least 0.1 and preferably at least 0.2; and the ratio of z/x is from 0.001 to about 0.5.

In one embodiment, and without wishing to be bound to any particular theory, it is believed that B moiety provides plasticity to the nanomagnetic material that it would not have but for the presence of such B moiety. In one aspect of this embodiment, it is preferred that the bending radius of a substrate coated with both A and B moieties be no greater than 90 percent of the bending radius of a substrate coated with only the A moiety.

The use of the B material allows one, in one embodiment, to produce a coated substrate with a springback angle of less than about 45 degrees. As is known to those skilled in the art, all materials have a finite modulus of elasticity; thus, plastic deformation is followed by some elastic recovery when the load is removed. In bending, this recovery is called springback. See, e.g., page 462 of S. Kalparjian's “Manufacturing Engineering and Technology,” Third Edition (Addison Wesley Publishing Company, New York, N.Y., 1995).

In one preferred embodiment, the B material is aluminum and the C material is nitrogen, whereby an AlN moiety is formed. Without wishing to be bound to any particular theory, applicants believe that aluminum nitride (and comparable materials) are both electrically insulating and thermally conductive, thus providing a excellent combination of properties for certain end uses.

Referring again to FIGS. 4 and 5, when an electromagnetic field 110 is incident upon the nanomagnetic material comprised of A and B (see FIG. 4), such a field will be reflected to some degree depending upon the ratio of moiety A and moiety B. In one embodiment, it is preferred that at least 1 percent of such field is reflected in the direction of arrow 112 (see FIG. 5). In another embodiment, it is preferred that at least about 10 percent of such field is reflected. In yet another embodiment, at least about 90 percent of such field is reflected. Without wishing to be bound to any particular theory, applicants believe that the degree of reflection depends upon the concentration of A in the A/B mixture.

Referring again to FIG. 4, and in one embodiment, the nanomagnetic material is comprised of moiety A, moiety C, and optionally moiety B. The moiety C is preferably selected from the group consisting of elemental oxygen, elemental nitrogen, elemental carbon, elemental fluorine, elemental chlorine, elemental hydrogen, and elemental helium, elemental neon, elemental argon, elemental krypton, elemental xenon, elemental fluorine, elemental sulfur, elemental hydrogen, elemental helium, the elemental chlorine, elemental bromine, elemental iodine, elemental boron, elemental phosphorus, and the like. In one aspect of this embodiment, the C moiety is selected from the group consisting of elemental oxygen, elemental nitrogen, and mixtures thereof.

In one embodiment, the C moiety is chosen from the group of elements that, at room temperature, form gases by having two or more of the same elements combine. Such gases include, e.g., hydrogen, the halide gases (fluorine, chlorine, bromine, and iodine), inert gases (helium, neon, argon, krypton, xenon, etc.), etc.

In one embodiment, the C moiety is chosen from the group consisting of oxygen, nitrogen, and mixtures thereof. In one aspect of this embodiment, the C moiety is a mixture of oxygen and nitrogen, wherein the oxygen is present at a concentration from about 10 to about 90 mole percent, by total moles of oxygen and nitrogen.

It is preferred, when the C moiety (or moieties) is present, that it be present in a concentration of from about 1 to about 90 mole percent, based upon the total number of moles of the A moiety and/or the B moiety and the C moiety in the composition. In one embodiment, the C moiety is both oxygen and nitrogen.

Referring again to FIG. 4, and in the embodiment depicted, the area 114 produces a composition which optimizes the degree to which magnetic flux are initially trapped and/or thereafter released by the composition when a magnetic field is withdrawing from the composition.

Without wishing to be bound to any particular theory, applicants believe that, when a composition as described by area 114 is subjected to an alternating magnetic field, at least a portion of the magnetic field is trapped by the composition when the field is strong, and then this portion tends to be released when the field lessens in intensity.

Thus, e.g., it is believed that, when the magnetic field 110 is applied to the nanomagnetic material, it starts to increase, in a typical sine wave fashion. After a specified period of time, a magnetic moment is created within the nanomagnetic material; but, because of the time delay, there is a phase shift.

The time delay will vary with the composition of the nanomagnetic material. By maximizing the amount of trapping, and by minimizing the amount of reflection and absorption, one may minimize the magnetic artifacts caused by the nanomagnetic shield.

Thus, and referring again to FIG. 4, one may optimize the A/B/C composition to preferably be within the area 114. In general, the A/B/C composition has molar ratios such that the ratio of A/(A and C) is from about 1 to about 99 mole percent and, preferably, from about 10 to about 90 mole percent. In one preferred embodiment, such ratio is from about 40 to about 60 molar percent.

The molar ratio of A/(A and B and C) generally is from about 1 to about 99 molar percent and, preferably, from about 10 to about 90 molar percent. In one embodiment, such molar ratio is from about 30 to about 60 molar percent.

The molar ratio of B/(A plus B plus C) generally is from about 1 to about 99 mole percent and, preferably, from about 10 to about 40 mole percent.

The molar ratio of C/(A plus B plus C) generally is from about 1 to about 99 mole percent and, preferably, from about 10 to about 50 mole percent.

In one embodiment, the composition of the nanomagnetic material is chosen so that the applied electromagnetic field 110 is absorbed by the nanomagnetic material by less than about 1 percent; thus, in this embodiment, the applied magnetic field 110 is substantially restored by correcting the time delay.

By utilizing nanomagnetic material that absorbs the electromagnetic field, one may selectively direct energy to various cells within a biological organism that are to treated. Thus, e.g., cancer cells can be injected with the nanomagnetic material and then destroyed by the application of externally applied electromagnetic fields. Because of the nano size of applicants' materials, they can readily and preferentially bedirected to the malignant cells to be treated within a living organism. In this embodiment, the nanomagnetic material preferably has a particle size of from about 5 to about 10 nanometers.

In one embodiment of this invention, there is provided a multiplicity of nanomagnetic particles that may be in the form of a film, a powder, a solution, etc. This multiplicity of nanogmentic particles is hereinafter referred to as a collection of nanomagnetic particles.

The collection of nanomagnetic particles of this embodiment of the invention is generally comprised of at least about 0.05 weight percent of such nanomagentic particles and, preferably, at least about 5 weight percent of such nanomagnetic particles. In one embodiment, such collection is comprised of at least about 50 weight percent of such magnetic particles. In another embodiment, such collection consists essentially of such nanomagnetic particles.

When the collection of nanomagnetic particles consists essentially of nanomagnetic particles, the term “compact” will be used to refer to such collection of nanomagnetic particles.

The average size of the nanomagnetic particles is preferably less than about 100 nanometers. In one embodiment, the nanomagnetic particles have an average size of less than about 20 nanometers. In another embodiment, the nanomagnetic particles have an average size of less than about 15 nanometers. In yet another embodiment, such average size is less than about 11 nanometers. In yet another embodiment, such average size is less than about 3 nanometers.

In one embodiment of this invention, the nanomagnetic particles have a phase transition temperature of from about 0 degrees Celsius to about 1,200 degees Celsius. In one aspect of this embodiment, the phase transition temperature is from about 40 degrees Celsius to about 200 degrees Celsius.

As used herein, the term phase transition temperature refers to temperature in which the magnetic order of a magnetic particle transitions from one magnetic order to another. Thus, for example, when a magnetic particle transitions from the ferromagnetic order to the paramagnetic order, the phase transition temperature is the Curie temperature. Thus, e.g., when the magnetic particle transitions from the anti-ferromagnetic order to the paramagnetic order, the phase transition temperature is known as the Neel temperature.

The nanomagnetic particles of this invention may be used for hyperthermia therapy. The use of small magnetic particles for hyperthermia therapy is discussed, e.g., in U.S. Pat. Nos. 4,136,683; 4,303,636; 4,735,796; and 5,043,101 of Robert T. Gordon. The entire disclosure of each of these Gordon patents is hereby incorporated by reference in to this specification.

U.S. Pat. No. 4,136,683 claims (claim 1) “A process for the measurement of the intracellular temperature of cells within the body comprising: intracellularly injecting into the patient, minute particles capable of magnetic characteristics and of the size less than 1 micron to permit absorbing said minute particles into the cells, determining the magnetic susceptibility of the intracellular particles with magnetic susceptibility measuring equipment and correlating the determined magnetic susceptibility to a corresponding temperature of the particles.”

U.S. Pat. No. 4,303,636 claims (claim 1) “1. A cancer treating composition for intravenous injection comprising: inductively heatable particles selected from the group consisting of ferromagnetic, paramagnetic and diamagnetic and of not greater than 1 micron suspended in an aqueous solution in dosage form.” It is disclosed in U.S. Pat. No. 4,303,636 that There are presently a number of methods and techniques for the treatment of cancer, among which may be included: radiation therapy, chemotherapy, immunotherapy, and surgery. The common characteristic for all of these techniques as well as any other presently known technique is that they are extracellular in scope, that is, the cancer cell is attacked and attempted to be killed through application of the killing force or medium outside of the cell.

U.S. Pat. No. 4,303,636 also discloses “This extracellular approach is found to be less effective and efficient because of the difficulties of penetrating the tough outer membrane of the cancer cell that is composed of two protein layers with a lipid layer in between. Of even greater significance is that to overcome the protection afforded the cell by the cell membrane in any extracellular technique, the attack on the cancer cells must be of such intensity that considerable damage is caused to the normal cells resulting in severe side effects upon the patient. Those side effects have been found to limit considerably the effectiveness and usefulness of these treatments.”

U.S. Pat. No. 4,303,636 also discloses that “A safe and effective cancer treatment has been the goal of investigators for a substantial period of time. Such a technique, to be successful in the destruction of the cancer cells, must be selective in effect upon the cancer cells and produce no irreversible damage to the normal cells. In sum, cancer treatment must selectively differentiate cancer cells from normal cells and must selectively weaken or kill the cancer cells without affecting the normal cells. It has been known that there are certain physical differences that exist between cancer cells and normal cells. One primary physical difference that exists is in the temperature differential characteristics between the cancer cells and the normal cells. Cancer cells, because of their higher rates of metabolism, have higher resting temperatures compared to normal cells. In the living cell, the normal temperature of the cancer cell is known to be 37.5° Centigrade, while that of the normal cell is 37° Centigrade. Another physical characteristic that differentiates the cancer cells from the normal cells is that cancer cells die at lower temperatures than do normal cells. The temperature at which a normal cell will be killed and thereby irreversibly will be unable to perform normal cell functions is a temperature of 46.5° Centigrade, on the average. The cancer cell, in contrast, will be killed at the lower temperature of 45.5° Centigrade. The temperature elevation increment necessary to cause death in the cancer cell is determined to be at least approximately 8.0° Centigrade, while the normal cell can withstand a temperature increase of at least 9.5° Centigrade.”

U.S. Pat. No. 4,303,636 also discloses “It is known, therefore, that with a given precisely controlled increment of heat, the cancer cells can be selectively destroyed before the death of the normal cells. On the basis of this known differential in temperature characteristics, a number of extracellular attempts have been made to treat cancer by heating the cancer cells in the body. This concept of treatment is referred to as hyperthermia. To achieve these higher temperatures in the cancer cells, researchers have attempted a number of methods including inducing high fevers, utilizing hot baths, diathermy, applying hot wax, and even the implanation of various heating devices in the area of the cancer. At this time, none of the various approaches to treat cancer have been truly effective and all have the common characteristic of approaching the problem by treating the cancer cell extracellularly. The outer membrane of the cancer cell, being composed of lipids and proteins, is a poor thermal conductor, thus making it difficult for the application of heat by external means to penetrate into the interior of the cell where the intracellular temperature must be raised to effect the death of the cell. If, through the extracellular approaches of the prior hyperthermia techniques, the temperatures were raised so high as to effect an adequate interacellular temperature to kill the cancer cells, many of the normal cells adjacent the application of heat could very well be destroyed.”

U.S. Pat. No. 4,735,796 claims (claim 1) “A diagnostic and disease treating composition comprising ferromagnetic, paramagnetic and diamagnetic particles not greater than about 1 micron in pharmacologically-acceptable dosage form, whereby magnetic charatieristics and chemical compositions of said particles are selected to provide an enhanced response on an electromagnetic field and to promote intracellular accumulation and compartmentalization of said particles resulting in increased sensitivity and effectiveness of diagnosis and of disease treatment based thereon, wherein said particles are metal transferrin dextran particles.” As is disclosed in U.S. Pat. No. 4,735,796, “The efficacy of minute particles possessing ferromagnetic, paramagnetic or diamagnetic properties for the treatment of disease, particularly cancer, has been described by R. T. Gordon in U.S. Pat. Nos. 4,106,488 and 4,303,636. As exemplified therein, ferric hydroxide and gallium citrate are used to form particles of a size of 1 micron or less and are introduced into cells in the area to be treated. All cells in the sample area are then subjected to a high frequency alternating electromagnetic field inductively heating the intracellular particles thus resulting in an increase in the intracellular temperature of the cells. Because the cancer cells accumulate the particles to a greater degree than the normal cells and further because of the higher ambient temperature of a cancer cell as compared to the normal cells; the temperature increase results in the death of the cancer cells but with little or no damage to normal cells in the treatment area. The particles are optionally used with specific cancer cell targeting materials (antibodies, radioisotopes and the like). Ferromagnetic, paramagnetic and diamagnetic particles have also been shown to be of value for diagnostic purposes. The ability of said particles to act as sensitive temperature indicators has been described in U.S. Pat. No. 4,136,683. The particles may also be used to enhance noninvasive medical scanning procedures (NMR imaging).”

U.S. Pat. No. 5,043,101 claims, in claim 1 thereof, “A method of manufacturing a metal-transferrin dextran compound comprising producing a metal transferrin compound by combining a solution of a metal salt with transferrin to obtain said metal transferrin compound; producing a metal dextran compound by combining a solution of a metal salt with dextran to obtain said metal dextran compound and combining said metal transferrin compound with said metal dextran compound to obtain said metal-transferrin dextran compound.” It is disclosed in U.S. Pat. No. 5,043,101 that: “This invention relates to the use of pharmacologically acceptable ferromagnetic, paramagnetic and diamagnetic particles in the diagnosis and treatment of disease. The particles possess magnetic properties uniquely suited for treatment and diagnostic regimens as disclosed in U.S. Pat. Nos. 4,106,488, 4,136,683 and 4,303,636. Enhanced magnetic properties displayed by the particles disclosed herein include favorable magnetic susceptibility and characteristic magnetic susceptibility vs. temperature profiles. The enhanced magnetic properties displayed by the particles result in increased sensitivity of response to an electromagnetic field thereby permitting a more sensitive application of diagnostic and treatment modalities based thereon. A further benefit is derived from the chemical composition of said particles whereby intracellular accumulation and compartmentalization of the particles is enhanced which also contributes to the more sensitive application of diagnostic and treatment modalities. Particles useful in light of the subject invention comprise inorganic elements and compounds as well as organic compounds such as metal-dextran complexes, metal-containing prosthetic groups, transport or storage proteins, and the like. The organic structures may be isolated from bacteria, fungi, plants or animals or may be synthesized in vitro from precursors isolated from the sources cited above.”

As suggested by the prior art, and by the instant specification, the nanomagnetic material of this invention is well adapted for hyperthermia therapy because, e.g., of the small size of the nanomagnetic particles and the magnetic properties of such particles, such as, e.g., their Curie temperature.

As used herein, the term “Curie temperature” refers to the temperature marking the transition between ferromagnetism and paramagnetism, or between the ferroelectric phase and paraelectric phase. This term is also sometimes referred to as the “Curie point.” Reference may be had, e.g., to U.S. Pat. Nos. 5,429,583, 6,599,234, 6,565,887, 6,267,313, 4,138,998, 5,571,153, 6,635,009, and the like. The entire disclosure of each of these U.S. patents is hereby incorporated by reference into this specification.

As used herein, the term “Neel temperature” refers to a temperature, characteristic of certain metals, alloys, and salts, below which spontaneous magnetic ordering takes place so that they become antiferromagnetic, and above which they are paramagnetic; this is also known as the Neel point. Reference may be had, e.g., to U.S. Pat. Nos. 4,103,315, 3,791,843, 5,492,720, 6,181,533, 3,883,892, 5,264,980, 3,845,306, 6,083,632, 4,396,886, 6,020,060, and the like. The entire disclosure of each of these U.S. patents is hereby incorporated by refernec into this specification.

Neel temperature is also disussed at page F-92 of the “Handbook of Chemistry and Physics,” 63^{rd} Edition (CRC Press, Inc., Boca Raton, Fla., 1982-1983). As is disclosed on such page, ferromagnetic materials are “those in which the magnetic moments of atoms or ions tend to assume an ordered but nonparallel arrangement in zero applied field, below a characteristic temperature called the Neel point. In the usual case, within a magnetic domain, a substantial net mangetization results form the antiparallel alignment of neighboring nonequivalent subslattices. The macroscopic behavior is similar to that in ferromagnetism. Above the Neel point, these materials become paramagnetic.”

As is disclosed in U.S. Pat. No. 5,412,182, the entire disclosure of which is hereby incorporated by reference into this specification, “The implants are accordingly heated by resistive loses from any induced current circulations and the tumor tissue is heated by thermal conduction. Implant temperatures are achieved in accordance with Curie temperature characteristics of the ferromagnetic material used. The ferromagnetic property of these implants changes as a function of temperature, heating is gradually reduced as the Curie temperature is approached and further reduced when the Curie temperature is exceeded. Thermal regulation is dependent on a sharp transition in the Curie temperature curve at the desired temperature. The availability of implants that can be thermally regulated at desirable temperatures is limited by practical metallurgy limitations. Further, coils used to generate required high intensity magnetic fields are extremely inefficient. In fact, 1500-3000 Watts can be required and the implants need to be aligned with the applied magnetic field. Due to the high power requirements, both very expensive radiofrequency shielded rooms and complex cooling systems are required.”

Without wishing to be bound to any particular theory, applicants believe that the phase temperature of their nanomagnetic particles can be varied by varying the ratio of the A, B, and C moieties described hereinabove as well as the particle sizes of the nanoparticles.

In one embodiment, the magnetic order of the nanomagnetic particles of this invention is destroyed at a temperature in excess of the phase transition temperature. This phenemon is illustrated in FIGS. 4A and 4B.

Referring to FIG. 4A, it will be seen that a multiplicity of nano-sized particles 91 are disposed within a cell 93 which, in the embodiment depicted, is a cancer cell. The particles 91 are subjected to electromagnetic radiation 95 which causes them, in the embodiment depicted, to heat to a temperature sufficient to destroy the cancer cell but insufficient to destroy surrounding cells. The particles 91 are preferably delivered to the cancer cell 93 by one or more of the means described elsewhere in this specification and/or in the prior art.

In the embodiment depicted in FIG. 4A, the temperature of the particles 91 is less than the phase transition temperature of such particles, “Tₜᵣₐₙₛᵢₜᵢₒₙ.” Thus, in this case, the particles 91 have a magnetic order, i.e., they are either ferromagnetic or superparamagnetic and, thus, are able to receive the external radiation 95 and transform at least a portion of the electromagnetic energy into heat.

When the temperature of the particles 91 exceeds the “Tₜᵣₐₙₛᵢₜᵢₒₙ” temperature (i.e., their phase transition temperature), the magnetic order of such particles is destroyed, and they are no longer able to transform electromagnetic energy into heat. This situation is depicted in FIG. 4B.

When the particles 91 cease transforming electromagnetic energy into heat, they tend to cool and then revert to a temperature below “Tₜᵣₐₙₛᵢₜᵢₒₙ”, as depicted in FIG. 4A. Thus, the particles 91 act as a heat switch, ceasing to transform electromagnetic energy into heat when they exceed their phase transition temperature and resuming such capability when they are cooled below their phase transition temperature. This capability is schematically illustrated in FIG. 3A.

In one embodiment, the phase transition temperature of the nanoparticles is higher than the temperature needed to kill cancer cells but lower than the temperature needed to kill normal cells. As is disclosed in, e.g., U.S. Pat. No. 4,776,086 (the entire disclosure of which is hereby incorporated by reference into this specification), “The use of elevated temperatures, i.e., hyperthermia, to repress tumors has been under continuous investigation for many years. When normal human cells are heated to 41°-43° C., DNA synthesis is reduced and respiration is depressed. At about 45° C., irreversible destruction of structure, and thus function of chromosome associated proteins, occurs. Autodigestion by the cell's digestive mechanism occurs at lower temperatures in tumor cells than in normal cells. In addition, hyperthermia induces an inflammatory response which may also lead to tumor destruction. Cancer cells are more likely to undergo these changes at a particular temperature. This may be due to intrinsic differences, between normal cells and cancerous cells. More likely, the difference is associated with the lop pH (acidity), low oxygen content and poor nutrition in tumors as a consequence of decreased blood flow. This is confirmed by the fact that recurrence of tumors in animals, after hyperthermia, is found in the tumor margins; probably as a consequence of better blood supply to those areas.”

In one embodiment of this invention, the phase transition temperature of the nanomagnetic material is less than about 50 degrees Celsius and, preferably, less than about 46 degrees Celsius. In one aspect of this embodiment, such phase transition temperature is less than about 45 degrees Celsius.

The nanomagnetic particles of this invention preferably have a saturation magnetization (“magnetic moment”) of from about 2 to about 3,000 electromagnetic units (emu) per cubic centimeter of material. This parameter may be measured by conventional means. Reference may be had, e.g., to U.S. Pat. No. 5,068,519 (magnetic document validator employing remanence and saturation measurements), U.S. Pat. Nos. 5,581,251, 6,666,930, 6,506,264 (ferromagnetic powder), U.S. Pat. Nos. 4,631,202, 4,610,911, 5,532,095, and the like. The entire disclosure of each of these U.S. patents is hereby incorporated by reference into this specification.

In one embodiment, the saturation magnetization of the nanomagnetic particles is measured by a SQUID (superconducting quantum interference device). Reference may be had, e.g., to U.S. Pat. No. 5,423,223 (fatigue detection in steel using squid mangetometry), U.S. Pat. No. 6,496,713 (ferromagnetic foreign body detection with background canceling), U.S. Pat. Nos. 6,418,335, 6,208,884 (noninvasive room temperature instrument to measure magnetic susceptibility variations in body tissue), U.S. Pat. No. 5,842,986 (ferromagnetic foreign body screening method), U.S. Pat. Nos. 5,471,139, 5,408,178, and the like. The entire disclosure of each of these U.S. patents is hereby incorporated by reference into this specification.

In one preferred embodiment, the saturation magnetization of the nanomagnetic particle of this invention is at least 100 electromagnetic units (emu) per cubic centimeter and, more preferably, at least about 200 electromagnetic units (emu) per cubic centimter. In one aspect of this embodiment, the saturation magnetization of such nanomagnetic particles is at least about 1,000 electromagnetic units per cubic centimeter.

In another embodiment, the nanomagnetic material of this invention is present in the form a film with a saturization magnetization of at least about 2,000 electromagnetic units per cubic centimeter and, more preferably, at least about 2,500 electromagnetic units per cubic centimeter. In this embodiment, the nanomagnetic material in the film preferably has the formula A₁A₂(B)ₓC₁(C₂)_{y}, wherein y is 1, and the C moieties are oxygen and nitrogen, respectively.

Without wishing to be bound to any particular theory, applicants believe that the saturation magnetization of their nanomagnetic particles may be varied by varying the concentration of the “magnetic” moiety A in such particles, and/or the concentrations of moieties B and/or C.

In one embodiment of this invention, the composition of one aspect of this invention is comprised of nanomagnetic particles with a specified magnetization. As is known to those skilled in the art, magnetization is the magnetic moment per unit volume of a substance. Reference may be had, e.g., to U.S. Pat. Nos. 4,169,998, 4,168,481, 4,166,263, 5,260,132, 4,778,714, and the like. The entire disclosure of each of these U.S. patents is hereby incorporated by reference into this specification.

In this embodiment, and in one aspect thereof, the nanomagnetic particles are present within a layer that preferably has a saturation magnetization, at 25 degrees Centigrade, of from about 1 to about 36,000 Gauss, or higher. In one embodiment, the saturation magnetization at room temperature of the nanomagentic particles is from about 500 to about 10,000 Gauss. For a discussion of the saturation magnetization of various materials, reference may be had, e.g., to U.S. Pat. Nos. 4,705,613, 4,631,613, 5,543,070, 3,901,741 (cobalt, samarium, and gadolinium alloys), and the like. The entire disclosure of each of these U.S. patents is hereby incorporated by reference into this specification. As will be apparent to those skilled in the art, especially upon studying the aforementioned patents, the saturation magnetization of thin films is often higher than the saturation magnetization of bulk objects.

In one embodiment, it is preferred to utilize a thin film with a thickness of less than about 2 microns and a saturation magnetization in excess of 20,000 Gauss. The thickness of the layer of nanomagentic material is measured from the bottom surface of the layer that contains such material to the top surface of such layer that contains such material; and such bottom surface and/or such top surface may be contiguous with other layers of material (such as insulating material) that do not contain nanomagnetic particles.

Thus, e.g., one may make a thin film in accordance with the procedure described at page 156 of Nature, Volume 407, Sep. 14, 2000, that describes a multilayer thin film that has a saturation magnetization of 24,000 Gauss.

By the appropriate selection of nanomagnetic particles, and the thickness of the films deposited, one may obtain saturation magnetizations of as high as at least about 36,000.

In one embodiment, the nanomagnetic materials used in the invention typically comprise one or more of iron, cobalt, nickel, gadolinium, and samarium atoms. Thus, e.g., typical nanomagnetic materials include alloys of iron and nickel (permalloy), cobalt, niobium, and zirconium (CNZ), iron, boron, and nitrogen, cobalt, iron, boron, and silica, iron, cobalt, boron, and fluoride, and the like. These and other materials are described in a book by J. Douglas Adam et al. entitled “Handbook of Thin Film Devices” (Academic Press, San Diego, Calif., 2000). Chapter 5 of this book,, beginning at page 185, describes “magnetic films for planar inductive components and devices;” and Tables 5.1 and 5.2 in this chapter describe many magnetic materials.

In one embodiment, the nanomagnetic material has a saturation magnetization of from about 1 to about 36,000 Gauss. In one embodiment, the nanomagnetic material has a saturation magnetization of from about 200 to about 26,000 Gauss.

In one embodiment, the nanomagnetic material also has a coercive force of from about 0.01 to about 5,000 Oersteds. The term coercive force refers to the magnetic field, H, which must be applied to a magnetic material in a symmetrical, cyclically magnetized fashion, to make the magnetic induction, B, vanish; this term often is referred to as magnetic coercive force. Reference may be had, e.g., to U.S. Pat. Nos. 4,061,824, 6,257,512, 5,967,223, 4,939,610, 4,741,953, and the like. The entire disclosure of each of these U.S. patents is hereby incorporated by reference into this specification.

In one embodiment, the nanomagnetic material has a coercive force of from about 0.01 to about 3,000 Oersteds. In yet another embodiment, the nanomagnetic material 103 has a coercive force of from about 0.1 to about 10.

In one embodiment, the nanomagnetic material preferably has a relative magnetic permeability of from about 1 to about 500,000; in one embodiment, such material has a relative magnetic permeability of from about 1.5 to about 260,000. As used in this specification, the term relative magnetic permeability is equal to B/H, and is also equal to the slope of a section of the magnetization curve of the magnetic material. Reference may be had, e.g., to page 4-28 of E. U. Condon et al.'s “Handbook of Physics” (McGraw-Hill Book Company, Inc., New York, 1958).

Reference also may be had to page 1399 of Sybil P. Parker's “McGraw-Hill Dictionary of Scientific and Technical Terms,” Fourth Edition (McGraw Hill Book Company, New York, 1989). As is disclosed on this page 1399, permeability is “. . . a factor, characteristic of a material, that is proportional to the magnetic induction produced in a material divided by the magnetic field strength; it is a tensor when these quantities are not parallel.

Reference also may be had, e.g., to U.S. Pat. Nos. 6,181,232, 5,581,224, 5,506,559, 4,246,586, 6,390,443, and the like. The entire disclosure of each of these U.S. patents is hereby incorporated by reference into this specification.

In one embodiment, the nanomagnetic material has a relative magnetic permeability of from about 1.5 to about 2,000.

In one embodiment, the nanomagnetic material preferably has a mass density of at least about 0.001 grams per cubic centimeter; in one aspect of this embodiment, such mass density is at least about 1 gram per cubic centimeter. As used in this specification, the term mass density refers to the mass of a give substance per unit volume. See, e.g., page 510 of the aforementioned “McGraw-Hill Dictionary of Scientific and Technical Terms.” In another embodiment, the material has a mass density of at least about 3 grams per cubic centimeter. In another embodiment, the nanomagnetic material has a mass density of at least about 4 grams per cubic centimeter.

In one embodiment, it is preferred that the nanomagnetic material, and/or the article into which the nanomagnetic material has been incorporated, be interposed between a source of radiation and a substrate to be protected therefrom.

In one embodiment, the nanomagnetic material is in the form of a layer that preferably has a saturation magnetization, at 25 degree Centigrade, of from about 1 to about 36,000 Gauss and, more preferably, from about 1 to about 26,000 Gauss. In one aspect of this embodiment, the saturation magnetization at room temperature of the nanomagnetic particles is from about 500 to about 10,000 Gauss.

In one embodiment, the nanomagnetic material is disposed within an insulating matrix so that any heat produced by such particles will be slowly dispersed within such matrix. Such matrix may be made from, e.g., ceria, calcium oxide, silica, alumina, and the like. In general, the insulating material preferably has a thermal conductivity of less than about 20 (calories centimeters/square centimeters-degree Kelvin second)×10,000. See,e.g., page E-6 of the 63^{rd}. Edition of the “Handbook of Chemistry and Physics” (CRC Press, Inc. Boca Raton, Fla., 1982).

In one embodiment, there is provided a coating of nanomagnetic particles that consists of a mixture of aluminum oxide (Al₂O₃), iron, and other particles that have the ability to deflect electromagnetic fields while remaining electrically non-conductive. In one aspect of this embodiment, the particle size in such a coating is approximately 10 nanometers. Preferably the particle packing density is relatively low so as to minimize electrical conductivity. Such a coating, when placed on a fully or partially metallic object (such as a guide wire, catheter, stent, and the like) is capable of deflecting electromagnetic fields, thereby protecting sensitive internal components, while also preventing the formation of eddy currents in the metallic object or coating. The absence of eddy currents in a metallic medical device provides several advantages, to wit: (1) reduction or elimination of heating, (2) reduction or elimination of electrical voltages which can damage the device and/or inappropriately stimulate internal tissues and organs, and (3) reduction or elimination of disruption and distortion of a magnetic-resonance image.

Determination of the Heat Shielding Effect of a Magnetic Shield

In one preferred embodiment, the composition of this invention minimizes the extent to which a substrate increases its heat when subjected to a strong magnetic filed. This heat buildup can be determined in accordance with A.S.T.M. Standard Test F-2182-02, “Standard test method for measurement of radio-frequency induced heating near passive implant during magnetic resonance imaging.”

In this test, the radiation used is representative of the fields present during MRI procedures. As is known to those skilled in the art, such fields typically include a static field with a strength of from about 0.5 to about 2 Teslas, a radio frequency alternating magnetic field with a strength of from about 20 microTeslas to about 100 microTeslas, and a gradient magnetic field that has three components (x, y, and z), each of which has a field strength of from about 0.05 to 500 milliTeslas.

During this test, a temperature probe is used to measure the temperature of an unshielded conductor when subjected to the magnetic field in accordance with such A.S.T.M. F-2182-02 test.

The same test is then is then performed upon a shielded conductor assembly that is comprised of the conductor and a magnetic shield.

The magnetic shield used may comprise nanomagnetic particles, as described hereinabove. Alternatively, or additionally, it may comprise other shielding material, such as, e.g., oriented nanotubes (see, e.g., U.S. Pat. No. 6,265,466).

In one embodiment, the shield is in the form of a layer of shielding material with a thickness of from about 10 nanometers to about 1 millimeter. In another embodiment, the thickness is from about 10 nanometers to about 20 microns.

In one preferred embodiment the shielded conductor is an implantable device and is connected to a pacemaker assembly comprised of a power source, a pulse generator, and a controller. The pacemaker assembly and its associated shielded conductor are preferably disposed within a living biological organism.

In one preferred embodiment, when the shielded assembly is tested in accordance with A.S.T.M. 2182-02, it will have a specified temperature increase (“dTₛ”). The “dT_{c}” is the change in temperature of the unshielded conductor using precisely the same test conditions but omitting the shield. The ratio of dTₛ/dT_{c} is the temperature increase ratio; and one minus the temperature increase ratio (1−dTₛ/dT_{c}) is defined as the heat shielding factor.

It is preferred that the shielded conductor assembly have a heat shielding factor of at least about 0.2. In one embodiment, the shielded conductor assembly has a heat shielding factor of at least 0.3.

In one embodiment, the nanomagnetic shield of this invention is comprised of an antithrombogenic material.

Antithrombogenic compositions and structures have been well known to those skilled in the art for many years. As is disclosed, e.g., in U.S. Pat. No. 5,783,570, the entire disclosure of which is hereby incorporated by reference into this specification, “Artificial materials superior in processability, elasticity and flexibility have been widely used as medical materials in recent years. It is expected that they will be increasingly used in a wider area as artificial organs such as artificial kidney, artificial lung, extracorporeal circulation devices and artificial blood vessels, as well as disposable products such as syringes, blood bags, cardiac catheters and the like. These medical materials are required to have, in addition to sufficient mechanical strength and durability, biological safety, which particularly means the absence of blood coagulation upon contact with blood, i.e., antithrombogenicity.”

“Conventionally employed methods for imparting antithrombogenicity to medical materials are generally classified into three groups of (1) immobilizing a mucopolysaccharide (e.g., heparin) or a plasminogen activator (e.g., urokinase) on the surface of a material, (2) modifying the surface of a material so that it carries negative charge or hydrophilicity, and (3) inactivating the surface of a material. Of these, the method of (1) (hereinafter to be referred to briefly as surface heparin method) is further subdivided into the methods of (A) blending of a polymer and an organic solvent-soluble heparin, (B) coating of the material surface with an organic solvent-soluble heparin, (C) ionical bonding of heparin to a cationic group in the material, and (D) covalent bonding of a material and heparin.”

“Of the above methods, the methods (2) and (3) are capable of affording a stable antithrombogenicity during a long-term contact with body fluids, since protein adsorbs onto the surface of a material to form a biomembrane-like surface. At the initial stage when the material has been introduced into the body (blood contact site) and when various coagulation factors etc. in the body have been activated, however, it is difficult to achieve sufficient antithrombogenicity without an anticoagulant therapy such as heparin administration.”

Other antithrombogenic methods and compositions are also well known. Thus, by way of further illustration, U.S. published patent application 20010016611 discloses an antithrombogenic composition comprising an ionic complex of ammonium salts and heparin or a heparin derivative, said ammonium salts each comprising four aliphatic alkyl groups bonded thereto, wherein an ammonium salt comprising four aliphatic alkyl groups having not less than 22 and not more than 26 carbon atoms in total is contained in an amount of not less than 5% and not more than 80% of the total ammonium salt by weight. The entire disclosure of this published patent application is hereby incorporated by reference into this specification.

Thus, e.g., U.S. Pat. No. 5,783,570 discloses an organic solvent-soluble mucopolysaccharide consisting of an ionic complex of at least one mucopolysaccharide (preferably heparin or heparin derivative) and a quaternary phosphonium, an antibacterial antithrombogenic composition comprising said organic solvent-soluble mucopolysaccharide and an antibacterial agent (preferably an inorganic antibacterial agent such as silver zeolite), and to a medical material comprising said organic solvent soluble mucopolysaccharide. The organic solvent-soluble mucopolysaccharide, and the antibacterial antithrombogenic composition and medical material containing same are said to easily impart antithrombogenicity and antibacterial property to a polymer to be a base material, which properties are maintained not only immediately after preparation of the material but also after long-term elution. The entire disclosure of this U.S. patent is hereby incorporated by reference into this specification.

By way of further illustration, U.S. Pat. No. 5,049,393 discloses anti-thrombogenic compositions, methods for their production and products made therefrom. The anti-thrombogenic compositions comprise a powderized anti-thrombogenic material homogeneously present in a solidifiable matrix material. The anti-thrombogenic material is preferably carbon and more preferably graphite particles. The matrix material is a silicon polymer, a urethane polymer or an acrylic polymer. The entire disclosure of this U.S. patent is hereby incorporated by reference into this specification.

By way of yet further illustration, U.S. Pat. No. 5,013,717 discloses a leach resistant composition that includes a quaternary ammonium complex of heparin and a silicone. A method for applying a coating of the composition to a surface of a medical article is also disclosed in the patent. Medical articles having surfaces that are both lubricious and antithrombogenic are produced in accordance with the method of the patent The entire disclosure of this U.S. patent is hereby incorporated by reference into this specification.

A Process for Preparation of an Iron-containing Thin Film

In one preferred embodiment of the invention, a sputtering technique is used to prepare an AlFe thin film or particles, as well as comparable thin films containing other atomic moieties, or particles, such as, e.g., elemental nitrogen, and elemental oxygen. Conventional sputtering techniques may be used to prepare such films by sputtering. See, for example, R. Herrmann and G. Brauer, “D.C.- and R.F. Magnetron Sputtering,” in the “Handbook of Optical Properties: Volume I—Thin Films for Optical Coatings,” edited by R. E. Hummel and K. H. Guenther (CRC Press, Boca Raton, Fla., 1955). Reference also may be had, e.g., to M. Allendorf, “Report of Coatings on Glass Technology Roadmap Workshop,” Jan. 18-19, 2000, Livermore, Calif.; and also to U.S. Pat. No. 6,342,134, “Method for producing piezoelectric films with rotating magnetron sputtering system.” The entire disclosure of each of these prior art documents is hereby incorporated by reference into this specification.

Although the sputtering technique is advantageously used, the plasma technique described elsewhere in this specification also may be used. Alternatively, or additionally, one or more of the other forming techniques described elsewhere in this specification also may be used.

One may utilize conventional sputtering devices in this process. By way of illustration and not limitation, a typical sputtering system is described in U.S. Pat. No. 5,178,739, the entire disclosure of which is hereby incorporated by reference into this specification. As is disclosed in this patent, “. . . a sputter system 10 includes a vacuum chamber 20, which contains a circular end sputter target 12, a hollow, cylindrical, thin, cathode magnetron target 14, a RF coil 16 and a chuck 18, which holds a semiconductor substrate 19. The atmosphere inside the vacuum chamber 20 is controlled through channel 22 by a pump (not shown). The vacuum chamber 20 is cylindrical and has a series of permanent, magnets 24 positioned around the chamber and in close proximity therewith to create a multiple field configuration near the interior surface 15 of target 12. Magnets 26, 28 are placed above end sputter target 12 to also create a multipole field in proximity to target 12. A singular magnet 26 is placed above the center of target 12 with a plurality of other magnets 28 disposed in a circular formation around magnet 26. For convenience, only two magnets 24 and 28 are shown. The configuration of target 12 with magnets 26, 28 comprises a magnetron sputter source 29 known in the prior art, such as the Torus-10E system manufactured by K. Lesker, Inc. A sputter power supply 30 (DC or RF) is connected by a line 32 to the sputter target 12. A RF supply 34 provides power to RF coil 16 by a line 36 and through a matching network 37. Variable impedance 38 is connected in series with the cold end 17 of coil 16. A second sputter power supply 39 is connected by a line 40 to cylindrical sputter target 14. A bias power supply 42 (DC or RF) is connected by a line 44 to chuck 18 in order to provide electrical bias to substrate 19 placed thereon, in a manner well known in the prior art.”

By way of yet further illustration, other conventional sputtering systems and processes are described in U.S. Pat. No. 5,569,506 (a modified Kurt Lesker sputtering system), U.S. Pat. No. 5,824,761 (a Lesker Torus 10 sputter cathode), U.S. Pat. Nos. 5,768,123, 5,645,910, 6,046,398 (sputter deposition with a Kurt J. Lesker Co. Torus 2 sputter gun), U.S. Pat. Nos. 5,736,488, 5,567,673, 6,454,910, and the like. The entire disclosure of each of these U.S. patents is hereby incorporated by reference into this specification.

By way of yet further illustration, one may use the techniques described in a paper by Xingwu Wang et al. entitled “Technique Devised for Sputtering AlN Thin Films,” published in “the Glass Researcher,” Volume 11, No. 2 (Dec. 12, 2002).

In one preferred embodiment, a magnetron sputtering technique is utilized, with a Lesker Super System III system The vacuum chamber of this system is preferably cylindrical, with a diameter of approximately one meter and a height of approximately 0.6 meters. The base pressure used is from about 0.001 to 0.0001 Pascals. In one aspect of this process, the target is a metallic FeAl disk, with a diameter of approximately 0.1 meter. The molar ratio between iron and aluminum used in this aspect is approximately 70/30. Thus, the starting composition in this aspect is almost non-magnetic. See, e.g., page 83 (FIG. 3.1aii) of R. S. Tebble et al.'s “Magnetic Materials” (Wiley-Interscience, New York, N.Y., 1969); this Figure discloses that a bulk composition containing iron and aluminum with at least 30 mole percent of aluminum (by total moles of iron and aluminum) is substantially non-magnetic.

In this aspect, to fabricate FeAl films, a DC power source is utilized, with a power level of from about 150 to about 550 watts (Advanced Energy Company of Colorado, model MDX Magnetron Drive). The sputtering gas used in this aspect is argon, with a flow rate of from about 0.0012 to about 0.0018 standard cubic meters per second. To fabricate FeAlN films in this aspect, in addition to the DC source, a pulse-forming device is utilized, with a frequency of from about 50 to about 250 MHz (Advanced Energy Company, model Sparc-le V). One may fabricate FeAlO films in a similar manner but using oxygen rather than nitrogen.

In this aspect, a typical argon flow rate is from about (0.9 to about 1.5)×10⁻³ standard cubic meters per second; a typical nitrogen flow rate is from about (0.9 to about 1.8)×10 standard cubic meters per second; and a typical oxygen flow rate is from about. (0.5 to about 2)×10⁻³ standard cubic meters per second. During fabrication, the pressure typically is maintained at from about 0.2 to about 0.4 Pascals. Such a pressure range has been found to be suitable for nanomagnetic materials fabrications. In one embodiment, it is preferred that both gaseous nitrogen and gaseous oxygen are present during the sputtering process.

In this aspect, the substrate used may be either flat or curved. A typical flat substrate is a silicon wafer with or without a thermally grown silicon dioxide layer, and its diameter is preferably from about 0.1 to about 0.15 meters. A typical curved substrate is an aluminum rod or a stainless steel wire, with a length of from about 0.10 to about 0.56 meters and a diameter of from (about 0.8 to about 3.0)×10⁻³ meters The distance between the substrate and the target is preferably from about 0.05 to about 0.26 meters.

In this aspect, in order to deposit a film on a wafer, the wafer is fixed on a substrate holder. The substrate may or may not be rotated during deposition. In one embodiment, to deposit a film on a rod or wire, the rod or wire is rotated at a rotational speed of from about 0.01 to about 0.1 revolutions per second, and it is moved slowly back and forth along its symmetrical axis with a maximum speed of about 0.01 meters per second.

In this aspect, to achieve a film deposition rate on the flat wafer of 5×10⁻¹⁰ meters per second, the power required for the FeAl film is 200 watts, and the power required for the FeAlN film is 500 watts The resistivity of the FeAlN film is approximately one order of magnitude larger than that of the metallic FeAl film. Similarly, the resistivity of the FeAlO film is about one order of magnitude larger than that of the metallic FeAl film.

Iron containing magnetic materials, such as FeAl, FeAlN and FeAlO, FeAlNO, FeCoAlNO, and the like, may be fabricated by sputtering. The magnetic properties of those materials vary with stoichiometric ratios, particle sizes, and fabrication conditions; see, e.g., R. S. Tebble and D. J. Craik, “Magnetic Materials”, pp. 81-88, Wiley-Interscience, New York, 1969 As is disclosed in this reference, when the iron molar ratio in bulk FeAl materials is less than 70 percent or so, the materials will no longer exhibit magnetic properties.

However, it has been discovered that, in contrast to bulk materials, a thin film material often exhibits different properties.

In one embodiment, the magnetic material A is dispersed within nonmagnetic material B. This embodiment is depicted schematically in FIG. 5.

Referring to FIG. 5, and in the preferred embodiment depicted therein, it will be seen that A moieties 102, 104, and 106 are preferably separated from each other either at the atomic level and/or at the nanometer level. The A moieties may be, e.g., A atoms, clusters of A atoms, A compounds, A solid solutions, etc. Regardless of the form of the A moiety, it preferably has the magnetic properties described hereinabove.

In the embodiment depicted in FIG. 5, each A moiety preferably produces an independent magnetic moment. The coherence length (L) between adjacent A moieties is, on average, preferably from about 0.1 to about 100 nanometers and, more preferably, from about 1 to about 50 nanometers.

Thus, referring again to FIG. 5, the normalized magnetic interaction between adjacent A moieties 102 and 104, and also between 104 and 106, is preferably described by the formula M=exp(−x/L), wherein M is the normalized magnetic interaction, exp is the base of the natural logarithm (and is approximately equal to 2.71828), x is the distance between adjacent A moieties, and L is the coherence length. M, the normalized magnetic interaction, preferably ranges from about 3×10⁻⁴⁴ to about 1.0. In one preferred embodiment, M is from about 0.01 to 0.99. In another preferred embodiment, M is from about 0.1 to about 0.9.

In one embodiment, and referring again to FIG. 5, x is preferably measured from the center 101 of A moiety 102 to the center 103 of A moiety 104; and x is preferably equal to from about 0.00001 times L to about 100 times L.

In one embodiment, the ratio of x/L is at least 0.5 and, preferably, at least 1.5.

In one embodiment, the “ABC particles” of nanomagentic material also have a specified coherence length. This embodiment is depicted in FIG. 5A.

As is used with regard to such “ABC particles,” the term “coherence length” refers to the smallest distance 1110 between the surfaces 113 of any particles 115 that are adjacent to each other. It is preferred that such coherence length, with regard to such ABC particles, be less than about 100 nanometers and, preferably, less than about 50 nanometers. In one embodiment, such coherence length is less than about 20 nanometers.

FIG. 6 is a schematic sectional view, not drawn to scale, of a shielded conductor assembly 130 that is comprised of a conductor 132 and, disposed around such conductor, a film 134 of nanomagnetic material. The conductor 132 preferably has a resistivity at 20 degrees Centigrade of from about 1 to about 100-microohom-centimeters.

The film 134 is comprised of nanomagnetic material that preferably has a maximum dimension of from about 10 to about 100 nanometers. The film 134 also preferably has a saturation magnetization of from about 200 to about 26,000 Gauss and a thickness of less than about 2 microns. In one embodiment, the magnetically shielded conductor assembly 130 is flexible, having a bend radius of less than 2 centimeters. Reference may be had, e.g., to U.S. Pat. No. 6,506,972, the entire disclosure of which is hereby incorporated by reference into this specification.

As used in this specification, the term flexible refers to an assembly that can be bent to form a circle with a radius of less than 2 centimeters without breaking. Put another way, the bend radius of the coated assembly is preferably less than 2 centimeters. Reference may be had, e.g., to U.S. Pat. Nos. 4,705,353, 5,946,439, 5,315,365, 4,641,917, 5,913,005, and the like. The entire disclosure of each of these U.S. patents is hereby incorporated by reference into this specification.

Without wishing to be bound to any particular theory, applicants believe that the use of nanomagnetic materials in their coatings and their articles of manufacture allows one to produce a flexible device that otherwise could not be produced were not the materials so used nano-sized (less than 100 nanometers).

Referring again to FIG. 6, and in the preferred embodiment depicted therein, one or more electrical filter circuit(s) 136 are preferably disposed around the nanomagnetic film 134. These circuit(s) may be deposited by conventional means.

In one embodiment, the electrical filter circuit(s) are deposited onto the film 134 by one or more of the techniques described in U.S. Pat. No. 5,498,289 (apparatus for applying narrow metal electrode), U.S. Pat. No. 5,389,573 (method for making narrow metal electrode), U.S. Pat. No. 5,973,573 (method of making narrow metal electrode), U.S. Pat. No. 5,973,259 (heated tool positioned in the X, Y, and 2-directions for depositing electrode), U.S. Pat. No. 5,741,557 (method for depositing fine lines onto a substrate), and the like. The entire disclosure of each of these U.S. patents is hereby incorporated by reference into this specification.

Referring again to FIG. 6, and in the preferred embodiment depicted therein, disposed around electrical filter circuit(s) 136 is a second film of nanomagnetic material 138, which may be identical to or different from film layer 134. In one embodiment, film layer 138 provides a different filtering response to electromagnetic waves than does film layer 134.

Disposed around nanomagnetic film layer 138 is a second layer of electrical filter circuit(s) 140. Each of circuit(s) 136 and circuit(s) 140 comprises at least one electrical circuit. It is preferred that the at least two circuits that comprise assembly 130 provide different electrical responses.

As is known to those skilled in the art, at high frequencies the inductive reactance of a coil is great. The inductive reactance (X_{L}) is equal to 2πFL, wherein F is the frequency (in hertz), and L is the inductance (in Henries).

At low-frequencies, by comparison, the capactitative reactance (X_{C}) is high, being equal to ½πFC, wherein C is the capacitance in Farads. The impedance of a circuit, Z, is equal to the square root of (R²+[X_{L}−X_{C}]²), wherein R is the resistance, in ohms, of the circuit, and X_{L} and X_{C} are the inductive reactance and the capacitative reactance, respectively, in ohms, of the circuit.

Thus, for any particular alternating frequency electromagnetic wave, one can, by the appropriate selection of values for R, L, and C, pick a circuit that is purely resistive (in which case the inductive reactance is equal to the capacitative reactance at that frequency), is primarily inductive, or is primarily capacitative.

Maximum power transfer occurs at resonance, when the inductance reactance is equal to the capactitative reactance and the difference between them is zero. Conversely, minimum power transfer occurs when the circuit has little resistance in it (all circuits have some finite resistance) but is predominantly inductive or predominantly capacitative.

An LC tank circuit is an example of a circuit in which minimum power is transmitted. A tank circuit is a circuit in which an inductor and capacitor are in parallel; such a circuit appears, e.g., in the output stage of a radio transmitter.

An LC tank circuit exhibits the well-known flywheel effect, in which the energy introduced into the circuit continues to oscillate between the capacitor and inductor after an input signal has been applied; the oscillation stops when the tank-circuit finally loses the energy absorbed, but it resumes when a new source of energy is applied. The lower the inherent resistance of the circuit, the longer the oscillation will continue before dying out.

A typical tank circuit is comprised of a parallel-resonant circuit; and it acts as a selective filter. As is known to those skilled in the art, and as is disclosed in Stan Gibilisco's “Handbook of Radio & Wireless Technology” (McGraw-Hill, New York, N.Y., 1999), a selective filter is a circuit designed to tailor the way an electronic circuit or system responds to signals at various frequencies (see page 62).

The selective filter may be a bandpass filter (see pages 62-63 of the Gibilisco book) that comprises a resonant circuit, or a combination of resonant circuits, designed to discriminate against all frequencies except a specified frequency, or a band of frequencies between two limiting frequencies. In a parallel LC circuit, a bandpass filter shows a high impedance at the desired frequency or frequencies and a low impedance at unwanted frequencies. In a series LC configuration, the filter has a low impedance at the desired frequency or frequencies, and a high impedance at unwanted frequencies.

The selective filter may be a band-rejection filter, also known as a band-stop filter (see pages 63-65 of the Gibilisco book). This band-rejection filter comprises a resonant circuit adapted to pass energy at all frequencies except within a certain range. The attenuation is greatest at the resonant frequency or within two limiting frequencies.

The selective filter may be a notch filter; see page 65 of the Gibilisco book. A notch filter is a narrowband-rejection filter. A properly designed notch filter can produce attenuation in excess of 40 decibels in the center of the notch.

The selective filter may be a high-pass filter; see pages 65-66 of the Gibilisco book. A high-pass filter is a combination of capacitance, inductance, and/or resistance intended to produce large amounts of attenuation below a certain frequency and little or no attenuation above that frequency. The frequency above which the transition occurs is called the cutoff frequency.

The selective filter may be a low-pass filter; see pages 67-68 of the Gibilisco book. A low-pass filter is a combination of capacitance, inductance, and/or resistance intended to produce large amounts of attenuation above a certain frequency and little or no attenuation below that frequency.

In the embodiment depicted in FIG. 6, the electrical circuit is preferably integrally formed with the coated conductor construct. In another embodiment, not shown in FIG. 6, one or more electrical circuits are separately formed from a coated substrate construct and then operatively connected to such construct.

FIG. 7A is a sectional schematic view of one preferred shielded assembly 131 that is comprised of a conductor 133 and, disposed around such conductor 133, a layer of nanomagnetic material 135.

In the embodiment depicted in FIG. 7A, the layer 135 of nanomagnetic material preferably has a thickness 137 of at least 150 nanometers and, more preferably, at least about 200 nanometers. In one embodiment, the thickness of layer 135 is from about 500 to about 1,000 nanometers.

The layer 135 of nanomagnetic material 137 preferably is comprised of nanomagnetic material that may be formed, e.g., by subjecting the material in layer 137 to a magnetic field of from about 10 Gauss to about 40 Tesla for from about 1 to about 20 minutes. The layer 135 preferably has a mass density of at least about 0.001 grams per cubic centimeter (and preferably at least about 0.01 grams per cubic centimeter), a saturation magnetization of from about 1 to about 36,000 Gauss, and a coercive force of from about 0.01 to about 5,000.

In one embodiment, the B moiety is added to the nanomagnetic A moiety, preferably with a B/A molar ratio of from about 5:95 to about 95:5 (see FIG. 3). In one aspect of this embodiment, the resistivity of the mixture of the B moiety and the A moiety is from about 1 micro-ohm-cm to about 10,000 micro-ohm-cm.

Without wishing to be bound to any particular theory, applicants believe that such a mixture of the A and B moieties provides two mechanisms for shielding the magnetic fields. One such mechanism/effect is the shielding provided by the nanomagnetic materials, described elsewhere in this specification. The other mechanism/effect is the shielding provided by the electrically conductive materials.

In one particularly preferred embodiment, the A moiety is iron, the B moiety is aluminum, and the molar ratio of A/B is about 70:30; the resistivity of this mixture is about 8 micro-ohms-cm.

FIG. 7B is a schematic sectional view of a magnetically shielded assembly 139 that is similar to assembly 131 but differs therefrom in that a layer 141 of nanoelectrical material is disposed around layer 135.

The layer of nanoelectrical material 141 preferably has a thickness of from about 0.5 to about 2 microns. In this embodiment, the nanoelectrical material comprising layer 141 has a resistivity of from about 1 to about 100 microohm-centimeters. As is known to those skilled in the art, when nanoelectrical material is exposed to electromagnetic radiation, and in particular to an electric field, it will shield the substrate over which it is disposed from such electrical field. Reference may be had, e.g., to International patent publication WO9820719 in which reference is made to U.S. Pat. No. 4,963,291; each of these patents and patent applications is hereby incorporated by reference into this specification.

As is disclosed in U.S. Pat. No. 4,963,291, one may produce electromagnetic shielding resins comprised of electroconductive particles, such as iron, aluminum,copper, silver and steel in sizes ranging from 0.5 to.50 microns. The entire disclosure of this U.S. patent is hereby incorporated by reference into this specification.

The nanoelectrical particles used in this aspect of the invention preferably have a particle size within the range of from about 1 to about 100 microns, and a resistivity of from about 1.6 to about 100 microohm-centimeters. In one embodiment, such nanoelectrical particles comprise a mixture of iron and aluminum. In another embodiment, such nanoelectrical particles consist essentially of a mixture of iron and aluminum.

It is preferred that, in such nanoelectrical particles, and in one embodiment, at least 9 moles of aluminum are present for each mole of iron. In another embodiment, at least about 9.5 moles of aluminum are present for each mole of iron. In yet another embodiment, at least 9.9 moles of aluminum are present for each mole of iron.

In one embodiment, and referring again to FIG. 7D, the layer 141 of nanoelectrical material has a thermal conductivity of from about 1 to about 4 watts/centimeter-degree Kelvin.

In one embodiment, not shown, in either or both of layers 135 and 141 there is present both the nanoelectrical material and the nanomagnetic material One may produce such a layer 135 and/or 141 by simultaneously depositing the nanoelectrical particles and the nanomagnetic particles with, e.g., sputtering technology such as, e.g., the sputtering technology described elsewhere in this specification.

FIG. 7C is a sectional schematic view of a magnetically shielded assembly 143 that differs from assembly 131 in that it contains a layer 145 of nanothermal material disposed around the layer 135 of nanomagnetic material. The layer 145 of nanothermal material preferably has a thickness of less than 2 microns and a thermal conductivity of at least about 150 watts/meter-degree Kelvin and, more preferably, at least about 200 watts/meter-degree Kelvin. It is preferred that the resistivity of layer 145 be at least about 10¹⁰ microohm-centimeters and, more preferably, at least about 10¹² microohm-centimeters. In one embodiment, the resistivity of layer 145 is at least about 10¹³ microohm centimeters. In one embodiment, the nanothermal layer is comprised of AIN.

In one embodiment, depicted in FIG. 7C, the thickness 147 of all of the layers of material coated onto the conductor 133 is preferably less than about 20 microns.

In FIG. 7D, a sectional view of an assembly 149 is depicted that contains, disposed around conductor 133, layers of nanomagnetic material 135, nanoelectrical material 141, nanomagnetic material 135, and nanoelectrical material 141.

In FIG. 7E, a sectional view of an assembly 151 is depicted that contains, disposed around conductor 133, a layer 135 of nanomagnetic material, a layer 141 of nanoelectrical material, a layer 135 of nanomagnetic material, a layer 145 of nanothermal material, and a layer 135 of nanomagnetic material. Optionally disposed in layer 153 is antithrombogenic material that is biocompatible with the living organism in which the assembly 151 is preferably disposed.

In the embodiments depicted in FIGS. 7A through 7E, the coatings 135, and/or 141, and/or 145, and/or 153, are disposed around a conductor 133. In one embodiment, the conductor so coated is preferably part of medical device, preferably an implanted medical device (such as, e.g., a pacemaker). In another embodiment, in addition to coating the conductor 133, or instead of coating the conductor 133, the actual medical device itself is coated.

A Preferred Sputtering Process

On Dec. 29, 2003, applicants filed U.S. patent application Ser. No. 10/747,472, for “Nanoelectrical Compositions.” The entire disclosure of this U.S. patent application is hereby incorporated by reference into this specification.

U.S. Ser. No. 10/747,472, at pages 10-15 thereof (and by reference to its FIG. 9), described the “. . . preparation of a doped aluminum nitride assembly.” This portion of U.S. Ser. No. 10/747,472 is specifically incorporated by reference into this specification. It is also described below, by reference to FIG. 8, which is similar to the FIG. 9 of U.S. Ser. No. 10/747,472 but utilizes different reference numerals.

The system depicted in FIG. 8 may be used to prepare an assembly comprised of moieties A, B, and C (see FIG. 4). FIG. 8 will be described hereinafter with reference to one of the preferred ABC moieties, i.e., aluminum nitride doped with magnesium.

FIG. 8 is a schematic of a deposition system 300 comprised of a power supply 302 operatively connected via line 304 to a magnetron 306. Disposed on top of magnetron 306 is a target 308. The target 308 is contacted by gas 310 and gas 312, which cause sputtering of the target 308. The material so sputtered contacts substrate 314 when allowed to do so by the absence of shutter 316.

In one preferred embodiment, the target 308 is mixture of aluminum and magnesium atoms in a molar ratio of from about 0.05 to about 0.5 Mg/(Al+Mg). In one aspect of this embodiment, the ratio of Mg/(Al+Mg) is from about 0.08 to about 0.12. These targets are commercially available and are custom made by companies such as, e.g., Kurt Lasker and Company of Pittsburgh, Pa.

The power supply 302 preferably provides pulsed direct current. Generally, power supply 302 provides power in excess of 300 watts, preferably in excess of 500 watts, and more preferably in excess of 1,000 watts. In one embodiment, the power supplied by power supply 302 is from about 1800 to about 2500 watts.

The power supply preferably provides rectangular-shaped pulses with a duration (pulse width) of from about 10 nanoseconds to about 100 nanoseconds. In one embodiment, the pulse width is from about 20 to about 40 nanoseconds.

In between adjacent pulses, preferably substantially no power is delivered. The time between adjacent pulses is generally from about 1 microsecond to about 10 microseconds and is generally at least 100 times greater than the pulse width. In one embodiment, the repetition rate of the rectangular pulses is preferably about 150 kilohertz.

One may use a conventional pulsed direct current (d.c.) power supply. Thus, e.g., one may purchase such a power supply from Advanced Energy Company of Colorado, and/or from ENI Company of Rochester, N.Y.

The pulsed d.c. power from power supply 302 is delivered to a magnetron 306, that creates an electromagnetic field near target 308. In one embodiment, a magnetic field has a magnetic flux density of from about 0.01 Tesla to about 0.1 Tesla.

As will be apparent, because the energy provided to magnetron 306 preferably comprises intermittent pulses, the resulting magnetic fields produced by magnetron 306 will also be intermittent. Without wishing to be bound to any particular theory, applicants believe that the use of such intermittent electromagnetic energy yields better results than those produced by continuous radio-frequency energy.

Referring again to FIG. 8, it will be seen that the process depicted therein preferably is conducted within a vacuum chamber 118 in which the base pressure is from about 1×10⁻⁸ Torr to about 0.000005 Torr. In one embodiment, the base pressure is from about 0.000001 to about 0.000003 Torr.

The temperature in the vacuum chamber 318 generally is ambient temperature prior to the time sputtering occurs.

In one aspect of the embodiment illustrated in FIG. 8, argon gas is fed via line 310, and nitrogen gas is fed via line 312 so that both impact target 308, preferably in an ionized state. In another embodiment of the invention, argon gas, nitrogen gas, and oxygen gas are fed via target 312.

The argon gas, and the nitrogen gas, are fed at flow rates such that the flow rate of the argon gas divided by the flow rate of the nitrogen gas preferably is from about 0.6 to about 1.2. In one aspect of this embodiment, such ratio of argon to nitrogen is from about 0.8 to about 0.95. Thus, for example, the flow rate of the argon may be 20 standard cubic centimeters per minute, and the flow rate of the nitrogen may be 23 standard cubic feet per minute.

The argon gas, and the nitrogen gas, contact a target 308 that is preferably immersed in an electromagnetic field. This field tends to ionize the argon and the nitrogen, providing ionized species of both gases. It is such ionized species that bombard target 308.

In one embodiment, target 308 may be, e.g., pure aluminum. In one preferred embodiment, however, target 308 is aluminum doped with minor amounts of one or more of the aforementioned moieties B.

In the latter embodiment, the moieties B are preferably present in a concentration of from about 1 to about 40 molar percent, by total moles of aluminum and moieties B. It is preferred to use from about 5 to about 30 molar percent of such moieties B.

The ionized argon gas, and the ionized nitrogen gas, after impacting the target 308, creates a multiplicity of sputtered particles 320. In the embodiment illustrated in FIG. 8 the shutter 316 prevents the sputtered particles from contacting substrate 314.

When the shutter 316 is removed, however, the sputtered particles 320 can contact and coat the substrate 314.

In one embodiment, illustrated in FIG. 8 the temperature of substrate 314 is controlled by controller 322 that can heat the substrate (by means such as a conduction heater or an infrared heater) and/or cool the substrate (by means such as liquid nitrogen or water).

The sputtering operation increases the pressure within the region of the sputtered particles 320. In general, the pressure within the area of the sputtered particles 320 is at least 100 times, and preferably 1000 times, greater than the base pressure.

Referring again to FIG. 8 a cryo pump 324 is preferably used to maintain the base pressure within vacuum chamber 318. In the embodiment depicted, a mechanical pump (dry pump) 326 is operatively connected to the cryo pump 324. Atmosphere from chamber 318 is removed by dry pump 326 at the beginning of the evacuation. At some point, shutter 328 is removed and allows cryo pump 324 to continue the evacuation. A valve 330 controls the flow of atmosphere to dry pump 326 so that it is only open at the beginning of the evacuation.

It is preferred to utilize a substantially constant pumping speed for cryo pump 324, i.e., to maintain a constant outflow of gases through the cryo pump 324. This may be accomplished by sensing the gas outflow via sensor 332 and, as appropriate, varying the extent to which the shutter 328 is open or partially closed.

Without wishing to be bound to any particular theory, applicants believe that the use of a substantially constant gas outflow rate insures a substantially constant deposition of sputtered nitrides.

Referring again to FIG. 8 and in one embodiment thereof, it is preferred to clean the substrate 314 prior to the time it is utilized in the process. Thus, e.g., one may use detergent to clean any grease or oil or fingerprints off the surface of the substrate. Thereafter, one may use an organic solvent such as acetone, isopropryl alcohol, toluene, etc.

In one embodiment, the cleaned substrate 314 is presputtered by suppressing sputtering of the target 308 and sputtering the surface of the substrate 314.

As will be apparent to those skilled in the art, the process depicted in FIG. 8 may be used to prepare coated substrates 314 comprised of moieties other than doped aluminum nitride.

FIG. 9 is a schematic, partial sectional illustration of a coated substrate 400 that, in the preferred embodiment illustrated, is comprised of a coating 402 disposed upon a stent 404. As will be apparent, only one side of the coated stent 404 is depicted for simplicity of illustration. As will also be apparent, the direct current magnetic susceptibility of assembly 400 is equal to the mass of stent (404)×(the susceptibility of stent 404)+the (nmass of the coating 402)×(the susceptibility of coating 402).

In the preferred coated substrate depicted in FIG. 9, the coating 402 may be comprised of one layer of material, two layers of material, or three or more layers of material.

Regardless of the number of coating layers used, it is preferred that the total thickness 410 of the coating 402 be at least about 400 nanometers and, preferably, be from about 400 to about 4,000 nanometers. In one embodiment, thickness 410 is from about 600 to about 1,000 nanometers. In another embodiment, thickness 410 is from about 750 to about 850 nanometers.

In the embodiment depicted, the substrate 404 has a thickness 412 that is substantially greater than the thickness 410. As will be apparent, the coated substrate 400 is not drawn to scale.

In general, the thickness 410 is less than about 5 percent of thickness 412 and, more preferably, less than about 2 percent. In one embodiment, the thickness of 410 is no greater than about 1.5 percent of the thickness 412.

The substrate 404, prior to the time it is coated with coating 402, has a certain flexural strength, and a certain spring constant.

The flexural strength is the strength of a material in bending, i.e., its resistance to fracture. As is disclosed in ASTM C-790, the flexural strength is a property of a solid material that indicates its ability to withstand a flexural or transverse load. As is known to those skilled in the art, the spring constant is the constant of proportionality k which appears in Hooke's law for springs. Hooke's law states that: F=−kx, wherein F is the applied force and x is the displacement from equilibrium. The spring constant has units of force per unit length.

Means for measuring the spring constant of a material are well known to those skilled in the art. Reference may be had, e.g., to U.S. Pat. No. 6,360,589 (device and method for testing vehicle shock absorbers), U.S. Pat. No. 4,970,645 (suspension control method and apparatus for vehicle), U.S. Pat. Nos. 6,575,020, 4,157,060, 3,803,887, 4,429,574, 6,021,579, and the like. The entire disclosure of each of these U.S. patents is hereby incorporated by reference into this specification.

Referring again to FIG. 9, the flexural strength of the uncoated substrate 404 preferably differs from the flexural strength of the coated substrate 404 by no greater than about 5 percent. Similarly, the spring constant of the uncoated substrate 404 differs from the spring constant of the coated substrate 404 by no greater than about 5 percent.

Referring again to FIG. 9, and in the preferred embodiment depicted, the substrate 404 is comprised of a multiplicity of openings through which biological material is often free to pass. As will be apparent to those skilled in the art, when the substrate 404 is a stent, it will be realized that the stent has a mesh structure.

FIG. 10 is a schematic view of a typical stent 500 that is comprised of wire mesh 502 constructed in such a manner as to define a multiplicity of openings 504. The mesh material is typically a metal or metal alloy, such as, e.g., stainless steel, Nitinol (an alloy of nickel and titanium), niobium, copper, etc.

Typically the materials used in stents tend to cause current flow when exposed to a field 506. When the field 506 is a nuclear magnetic resonance field, it generally has a direct current component, and a radio-frequency component. For MRI (magnetic resonance imaging) purposes, a gradient component is added for spatial resolution.

The material or materials used to make the stent itself has certain magnetic properties such as, e.g., magnetic susceptibility. Thus, e.g., niobium has a magnetic susceptibility of 1.95×10⁻⁶ centimeter-gram-second units. Nitonol has a magnetic susceptibility of from about 2.5 to about 3.8×10⁻⁶ centimeter-gram-second units. Copper has a magnetic susceptibility of from −5.46 to about −6.16×10⁻⁶ centimeter-gram-second units.

The total magnetic susceptibility of an object is equal to the mass of the object times its succeptibility. Thus, assuming an object has equal parts of niobium, Nitinol, and copper, its total susceptibility would be equal to (+1.95+3.15−5.46)×10⁻⁶ cgs, or about 0.36×10⁻⁶ cgs.

In a more general case, where the masses of niobium, Nitinol, and copper are not equal in the object, the susceptibility, in c.g.s. units, would be equal to 1.95 Mn+3.15 Mni−5.46 Mc, wherein Mn is the mass of niobium, Mni is th mass of Nitinol, and Mc is the mass of copper.

When any particular material is used to make the stent, its response to an applied MRI field will vary depending upon, e.g., the relative orientation of the stent in relationship to the fields (including the d.c. field, the r.f. field, an the gradient field).

Any particular stent implanted in a human body will tend to have a different orientation than any other stent implanted in another human body due, in part, to the uniqueness of each human body. Thus, it cannot be predicted a priori how any particular stent will respond to a particular MRI field.

The solution provided by one aspect of applicants' invention tends to cancel, or compensate for, the response of any particular stent in any particular body when exposed to an MRI field.

Referring again to FIG. 10, and to the uncoated stent 500 depicted therein, when an MRI field 506 is imposed upon the stent, it will tend to induce eddy currents. As used in this specification, the term eddy currents refers to loop currents and surface eddy currents.

Referring to FIG. 10, the MRI field 506 will induce a loop current 508. As is apparent to those skilled in the art, the MRI field 506 is an alternating current field that, as it alternates, induces an alternating eddy current 508. The radio-frequency field is also an alternating current field, as is the gradient field. By way of illustration, when the d.c. field is about 1.5 Tesla, the r.f. field has frequency of about 64 megahertz. With these conditions, the gradient field is in the kilohertz range, typically having a frequency of from about 2 to about 200 kilohertz.

Applying the well-known right hand rule, the loop current 508 will produce a magnetic field 510 extending into the plane of the paper and designated by an “x.” This magnetic field 510 will tend to oppose the direction of the applied field 506.

Referring again to FIG. 10, when the stent 500 is exposed to the MRI field 506, a surface eddy current will be produced where there is a relatively large surface area of conductive material such as, e.g., at junction 514.

The stent 500 shoulud be constructed to have certain desirable mechanical properties. However, the materials that will provide the desired mechanical properties generally do not have desirable magnetic and/or electromagnetic properties. In an ideal situation, the stent 500 will produce no loop currents 508 and no surface eddy currents 512; in such situation, the stent 500 would have an effective zero magnetic susceptibility. Put another way, ideally the direct current magnetic susceptibility of an ideal stent should be about 0.

A d.c. (“direct current”) magnetic susceptibility of precisely zero is often difficult to obtain. In general, it is sufficient if the d.c. susceptibility of the stent is plus or minus 1×10⁻³ centimeter-gram-seconds (cgs) and, more preferably, plus or minus 1×10⁻⁴ centimeter-gram-seconds. In one embodiment, the d.c. susceptibility of the stent is equal to plus or minus 1×10⁻⁵ centimeter-gram-seconds. In another embodiment, the d.c. susceptibility of the stent is equal to plus or minus 1×10⁻⁶ centimeter-gram-seconds.

In one embodiment, discussed elsewhere in this specification the d.c. susceptibilility of the stent in contact with bodily fluid is plus or minus plus or minus 1×10⁻³ centimeter-gram-seconds (cgs), or plus or minus 1×10⁻⁴ centimeter-gram-seconds, or plus or minus 1×10⁻⁵ centimeter-gram-seconds, or plus or minus 1×10⁻⁶ centimeter-gram-seconds. In this embodiment, the materials comprising the nanomagnetic coating on the stent are chosen to have susceptibility values that, in combination with the susceptibility values of the other components of the stent, and of the bodily fluid, will yield the desired values.

The prior art has heretofore been unable to provide such an ideal stent. Applicants' invention allows one to compensate for the deficiencies of the current stents, and/or of the current stents in contact with bodily fluid, by canceling the undesirable effects due to their magnetic susceptibilities, and/or by compensating for such undesirable effects.

FIG. 11 is a graph of the magnetization of an object (such as an uncoated stent, or a coated stent) when subjected to an electromagnetic filed, such as an MRI field. It will be seen that, at different field strengths, different materials have different magnetic responses.

Thus, e.g., it will be seen that copper, at a d.c. field strength of 1.5 Tesla, is changing its magnetization as a function of the composite field strength (including the d.c. field strength, the r.f. field strength, and the gradient field strength) at a rate (defined by delta-magnetization/delta composite field strength) that is decreasing. With regard to the r.f. field and the gradient field, it should be understood that the order of magnitude of these fields is relatively small compared to the d.c. field, which is usually about 1.5 Tesla.

Referring again to FIG. 11, it will be seen that the slope of line 602 is negative. This negative slope indicates that copper, in response to the applied fields, is opposing the applied fields. Because the applied fields (including r.f. fields, and the gradient fields), are required for effective MRI imaging, the response of the copper to the applied fields tends to block the desired imaging, especially with the loop current and the surface eddy current described hereinabove. The d.c. susceptibility of copper is equal to the mass of the copper preent in the device times its magnetic susceptibility.

Referring again to FIG. 11, and in the preferred embodiment depicted therein, the ideal magnetization response is illustrated by line 604, which is the response of the coated substrate of one aspect of this invention, and wherein the slope is substantially zero. As used herein, and with regard to FIG. 11, the term substantially zero includes a slope will produce an effective magnetic susceptibility of from about 1×10⁻⁷ to about 1×10⁻⁸ centimeters-gram-second (cgs).

Referring again to FIG. 11, one means of correcting the negative slope of line 602 is by coating the copper with a coating which produces a response 606 with a positive slope so that the composite material produces the desired effective magnetic susceptibility of from about 1×10⁻⁷ to about 1×10⁻⁸ centimeters-gram-second (cgs) units. In order to do so, the following equation must be satisfied: (magnetic susceptibility of the uncoated device) (mass of uncoated device)+(magnetic susceptibility of copper)(mass of copper)=from about 1×10⁻⁷ to about 1×10⁻⁸ centimeters-gram-second (cgs).

FIG. 9 illustrates a coating that will produce the desired correction for the copper substrate 404. Referring to FIG. 9, it will be seen that, in the embodiment depicted, the coating 402 is comprised of at least nanomagnetic material 420 and nanodielectric material 422.

In one embodiment, the nanomagnetic material 402 preferably has an average particle size of less than about 20 nanometers and a saturation magnetization of from 10,000 to about 26,000 Gauss.

In one embodiment, the nanomagnetic material used is iron. In another embodiment, the nanomagentic material used is FeAlN. In yet another embodiment, the nanomagnetic material is FeAl. Other suitable materials will be apparent to those skilled in the art and include, e.g., nickel, cobalt, magnetic rare earth materials and alloys, thereof, and the like.

The nanodielectric material 422 preferably has a resistivity at 20 degrees Centigrade of from about 1×10⁻⁵ ohm-centimeters to about 1×10¹³ ohm-centimeters.

Referring again to FIG. 9, and in the preferred embodiment depicted therein, the nanomagnetic material 420 is preferably homogeneously dispersed within nanodielectric material 422, which acts as an insulating matrix. In general, the amount of nanodielectric material 422 in coating 402 exceeds the amount of nanomagnetic material 420 in such coating 402. In general, the coating 402 is comprised of at least about 70 mole percent of such nanodielectric material (by total moles of nanomagnetic material and nanodielectric material). In one embodiment, the coating 402 is comprised of less than about 20 mole percent of the nanomagnetic material, by total moles of nanomagnetic material and nanodielectric material. In one embodiment, the nanodielectric material used is aluminum nitride.

Referring again to FIG. 9, one may optionally include nanoconductive material 424 in the coating 402. This nanoconductive material generally has a resistivity at 20 degrees Centigrade of from about 1×10⁻⁶ ohm-centimeters to about 1×10⁻⁵ ohm-centimeters; and it generally has an average particle size of less than about 100 nanometers. In one embodiment, the nanoconductive material used is aluminum.

Referring again to FIG. 9, and in the embodiment depicted, it will be seen that two layers are preferably used to obtain the desired correction. In one embodiment, three or more such layers are used. This embodiment is depicted in FIG. 9A.

FIG. 9A is a schematic illustration of a coated substrate that is similar to coated substrate 400 but differs therefrom in that it contains two layers of dielectric material 405 and 407. In one embodiment, only one such layer of dielectric material 405 issued. Notwithstanding the use of additional layers 405 and 407, the coating 402 still preferably has a thickness 410 of from about 400 to about 4000 nanometers In the embodiment depicted in FIG. 9A, the direct current susceptibility of the assembly depicted is equal to the sum of the (mass)×(susceptibility) for each individual layer.

As will be apparent, it may be difficult with only one layer of coating material to obtain the desired correction for the material comprising the stent (see FIG. 11). With a multiplicity of layers comprising the coating 402, which may have the same and/or different thicknesses, and/or the same and/or different masses, and/or the same and/or different compositions, and/or the same and/or different magnetic susceptibilities, more flexibility is provided in obtaining the desired correction.

FIG. 11 illustrates the desired correction in terms of magnetization. FIG. 12 illustrates the desired correction in terms of reactance.

Referring again to FIG. 11, in the embodiment depicted a correction is shown for a coating on a substrate. As will be apparent, the same correction can be made with a mixture of at least two different materials in which each of the different materials retains its distinct magnetic characteristics, and/or any composition containing at least two different moieties, provided that each of such different moieties retains its distinct magnetic characteristics. Such correction process is illustrated in FIG. 1I A.

FIG. 11A illustrates the response of different species within a composition (such as, e.g., a particle) to magnetic radiation, wherein each such species retains its individual magnetic characteristics. The graph depicted in FIG. 11A does not illustrate the response of different species alloyed with each other, wherein each of the species does not retain its individual magnetic characteristics.

As is known to those skilled in the art, an alloy is a substance having magnetic properties and consisting of two or more elements, which usually are metallic elements. The bonds in the alloy are usually metallic bonds, and thus the individual elements in the alloy do not retain their individual magnetic properties because of the substantial “crosstalk” between the elements via the metallic bonding process.

By comparison, e.g., materials that are covalently bond to each other are more likely to retain their individual magnetic characteristics; it is such materials whose behavior is illustrated in FIG. 11A. Each of the “magnetically distinct” materials may be, e.g., a material in elemental form, a compound, an alloy, etc.

Referring again to FIG. 11A, the response of different, “magnetically distinct” species within a composition (such as particle compact) to MRI radiation is shown. In the embodiment depicted, a direct current (d.c.) magnetic field is shown being applied in the direction of arrow 701. The magnetization plot 703 of the positively magnetized species is shown with a positive slope.

As is known to those skilled in the art, the positively magnetized species include, e.g., those species that exhibit paramagetism, superparamagnetism, ferromagnetism, and/or ferrimagnetism.

Paramagnetism is a property exhibited by substances which, when placed in a magnetic field, are magnetized parallel to to the field to an extent proportional to the field (except at very low temperatures or in extrely large magnetic fields). Paramagnetic materials are well known to those skilled in the art. Reference may be had, e.g., to U.S. Pat. No. 5,578,922 (paramagnetic material in solution), U.S. Pat. No. 4,704,871 (magnetic refrigeration apparatus with belt of paramagnetic material), U.S. Pat. No. 4,243,939 (base paramagnetic material containing ferromagnetic impurity), U.S. Pat. No. 3,917,054 (articles of paramagnetic material), U.S. Pat. No. 3,796,4999 (paramagnetic material disposed in a gas mixture), and the like. The entire disclosure of each of these U.S. patents is hereby incorporated by reference into this specification.

Superparamagnetic materials are also well known to those skilled in the art. Reference may be had, e.g., to U.S. Pat. No. 5,238,811, the entire disclosure of which is hereby incorporated by reference into this specification, it is disclosed (at column 5) that: “The superparamagnetic material used in the assay methods according to the first and second embodiments of the present invention described above is a substance which has a particle size smaller than that of a ferromagnetic material and retains no residual magnetization after disappearance of the external magnetic field. The superparamagnetic material and ferromagnetic material are quite different from each other in their hysteresis curve, susceptibility, Mesbauer effect, etc. Indeed, ferromagnetic materials are most suited for the conventional assay methods since they require that magnetic micro-particles used for labeling be efficiently guided even when a weak magnetic force is applied. On the other hand, in the non-separation assay method according to the first embodiment of the present invention, it is required that the magnetic-labeled body alone be difficult to guide by a magnetic force, and for this purpose superparamagnetic materials are most suited.” The preparation of these superparamagnetic materials is discussed at columns 6 et seq. of U.S. Pat. No. 5,238,811, wherein it is disclosed that: “The ferromagnetic substances can be selected appropriately, for example, from various compound magnetic substances such as magnetite and gamma-ferrite, metal magnetic substances such as iron, nickel and cobalt, etc. The ferromagnetic substances can be converted into ultramicro particles using conventional methods excepting a mechanical grinding method, i.e., various gas phase methods and liquid phase methods. For example, an evaporation-in-gas method, a laser heating evaporation method, a coprecipitation method, etc. can be applied. The ultramicro particles produced by the gas phase methods and liquid phase methods contain both superparamagnetic particles and ferromagnetic particles in admixture, and it is therefore necessary to separate and collect only those particles which show superparamagnetic property. For the separation and collection, various methods including mechanical, chemical and physical methods can be applied, examples of which include centrifugation, liquid chromatography, magnetic filtering, etc. The particle size of the superparamagnetic ultramicro particles may vary depending upon the kind of the ferromagnetic substance used but it must be below the critical size of single domain particles. Preferably, it is not larger than 10 nm when the ferromagnetic substance used is magnetite or gamma-ferrite and it is not larger than 3 nm when pure iron is used as a ferromagnetic substance, for example.”

Ferromagnetic materials may also be used as the positively magnetized species. As is known to those skilled in the art, ferromagnetism is a property, exhibited by certain metals, alloys, and compounds of the transition (iron group), rare-earth, and actinide elements, in which the internal magnetic moments spontaneously organize in a common direction; this property gives rise to a permeability considerably greater than that of a cuum, and also to magnetic hysteresis. Reference may be had, e.g., to U.S. Pat. Nos. 6,475,650; 6,299,990; 6,690,287 (ferromagnetic material having improved impedance matching); U.S. Pat. No. 6,366,083 (crud layer containing ferromagnetic material on nuclear fuel rods); U.S. Pat. No. 6,011,674 (magnetoreisstance effect multilayer film with ferromagnetic film sublayers of different ferromagnetic material compositions); U.S. Pat. No. 5,648,015 (process for preparing ferromagnetic materials); U.S. Pat. Nos. 5,382,304; 5,272,238 (organo-ferromagnetic material); U.S. Pat. No. 5,247,054 (organic polymer ferromagnetic material); U.S. Pat. No. 5,030,371 (acicular ferromagnetic material consisting essentially of iron-containing chromium dioxide); U.S. Pat. No. 4,917,736 (passive ferromagnetic material); U.S. Pat. No. 4,863,715 (contrast agent comprising particles of ferromagnetic material); U.S. Pat. No. 4,835,510 (magnetoresistive element of ferromagnetic material); U.S. Pat. No. 4,739,294 (amorphous and non-amorphous ferromagnetic material); U.S. Pat. No. 4,289,937 (fine grain ferromagnetic material); U.S. Pat. No. 4,023,412 (the Curie point of a ferromagnetic material); U.S. Pat. No. 4,015,030 (stabilized ferromagnetic material); U.S. Pat. No. 4,004,997 (a polymerizable compostion containing a magnetized powdered ferromagnetic material); U.S. Pat. No. 3,851,375 (sintered oxidic ferromagnetic material); U.S. Pat. No. 3,850,706 (ferromagnetic materials comprised of transition metals); and the like. The entire disclosure of each of these U.S. patents is hereby incorporated by reference into this specification.

Ferrimagnetic materials may also be used as the positively magnetized specifies. As is known to those skilled in the art, ferrimagnetism is a type of magnetism in which the magnetic moments of neighboring ions tend to align nonparallel, usually antiparallel, to each other, but the moments are of different magnitudes, so there is an appreciable, resultant magnetization. Reference may be had, e.g., to U.S. Pat. Nos. 6,538,919; 6,056,890 (ferrimagnetic materials with temperature stability); U.S. Pat. Nos. 4,649,495; 4,062,920 (lithium-containing ferrimagnetic materials); U.S. Pat. Nos. 4,059,664; 3,947,372 (ferromagnetic material); U.S. Pat. No. 3,886,077 (garnet structure ferromagnetic material); U.S. Pat. Nos. 3,765,021; 3,670,267; and the like. The entire disclosure of each of these U.S. patents is hereby incorporated by reference into this specification.

A discussion of certain paramagnetic, superparamagnetic, ferromagnetic, and/or ferromagnetic materials is presented in U.S. Pat. No. 5,238,811, the entire disclosure of which is hereby incorporated by reference into this specification. As is disclosed in this patent, “. . . The superparamagnetic ultrarnicro particles can be produced from any ferromagnetic substances, by rendering them ultramicro particles. The ferromagnetic substances can be selected appropriately, for example, from various compound magnetic substances such as magnetite and gamma-ferrite, metal magnetic substances such as iron, nickel and cobalt, etc The ferromagnetic substances can be converted into ultrarnicro particles using conventional methods excepting a mechanical grinding method, i.e., various gas phase methods and liquid phase methods . . . . ”

“The particle size of the superparamagnetic ultramicro particles may vary depending upon the kind of the ferromagnetic substance used but it must be below the critical size of single domain particles. Preferably, it is not larger than 10 nm when the ferromagnetic substance used is magnetite or gamma-ferrite and it is not larger than 3 nm when pure iron is used as a ferromagnetic substance, for example.”

“As is well known, ferromagnetic particles are converted to superparamagnetic particles according as their particle size is reduced greatly since the direction of easy magnetization thereof becomes random due to the influence of thermal movement. Taking magnetite particles as an example, it is known that they are converted to a mixture of ferromagnetic particles and superparamagnetic particles when their particle size is reduced to 10 nm or less. The ferromagnetism and superparamagnetism can readily be distinguished by measuring their hysteresis curves or susceptibility, or by Mesbauer effects. That is, the coercive force of superparamagnetic substances is zero and their susceptibility decreases as their particle size decreases since the influence of the particle size on the susceptibility is reversed at the critical particle size at which ferromagnetism is converted to superparamagnetism. In ferromagnetism a Mesbauer spectrum of iron is divided into 6 lines in contrast to superparamagnetism in which two absorption lines appear in the center, which enables quantitative determination of superparamagnetism. The thermal magnetic relaxation time in which magnetization is reversed due to thermal agitation is calculated to be 1 second at a particle size of 2.9 nm and about 109 seconds or about 30 years at a particle size of 3.6 nm in the case of ultramicro particles of iron at room temperature when no external magnetic field is applied. This clearly shows that difference in the particle size of only 1 nm results in drastic change in the magnetic property.”

“Giaever, U.S. Pat. No. 3,970,518, “Magnetic Separation of Biological Particles”, discloses a method of separating cells or the like by coating ferromagnetic or ferrimagnetic materials such as ferrite, perovskite, chromite, magnetoplumbite, etc. having a size in the range between the size of colloid particles and 10 micrometers with an antibody. (4) Davies, et al., U.S. Pat. No. 4,177,253, “Magnetic Particle for Immunoassay”, describes composite magnetic particles having a particle size of 1 micrometer to 1 cm and comprising a core material of a low density coated on the surface thereof with a metal magnetic-material such as Ni, etc., and a biologically active substance such as an antigen or antibody. (5) Molday, U.S. Pat. No. 4,452,773, “Magnetic Iron-Dextran Microspheres”, describes dextran-coated micro-particles of magnetite, which is one of ferromagnetic substances having a particle size of preferably 30 to 40 nm. (6) Czerlinski, U.S. Pat. No. 4,454,234, “Coated Magnetizable Microparticles, Reversible Suspensions Thereof, and Processes Relating Thereto”, describes magnetic micro-particles having a particle size in the range between the size of magnetic domain and about 0.1 micrometer and comprising micro-particles of a ferromagnetic material such as ferrite, yttrium-iron-garnet, etc. whose Curie temperature is in the range between 5 degree C. to 65 degree C. and whose surface is coated with a copolymer composition based on acrylamide. (7) Ikeda, et al., U.S. Pat. No. 4,582,622, “Magnetic Particulate for Immobilization of Biological Protein and Process of Producing the Same”, describes particles of a particle size of about 3 micrometers composed mainly of gelatin and containing 0.00001% to 2% ferromagnetic substance composed of ferrite. (8) Margel, U.S. Pat. No. 4,324,923, “Metal Coated Polyaldehyde Microspheres”, escribes polyaldehyde microspheres coated with a transient metal and containing ferromagnetic substance such as iron, nickel, cobalt, etc. as a magnetic material. The magnetic materials described in (4) to (8) above each are ferromagnetic or ferrimagnetic particles having a particle size of at least 30 nm, and are classified under as ferromagnetic materials. Ferromagnetic materials are those having a particle size of usually several tens nm or more, which may vary depending on the kind of the material, and showing residual magnetization after disappearance of an external magnetic field.”

“The superparamagnetic ultramicro-particles 1 are ultramicro-particles of iron having a mean particle size of 2 nm, whose surface is coated with protein A. The iron ultramicro-particles were prepared by conventional vacuum evaporation method, and a magnetic field filter was used to separate those particles with superparamagnetic property from those with ferromagnetic property in order to recover only superparamagnetic particles.”

By way of yet further illustration, and not limitation, some suitable positively magnetized species include, e.g., iron; iron/aluminum; iron/aluminum oxide; iron/aluminum nitride; iron/tantalum nitride; iron/tantalum oxide; nickel; nickel/cobalt; cobalt/iron; cobalt; samarium; gadolinium; neodymium; mixtures thereof; nano-sized particles of the aforementioned mixtures, where super-paramagnetic properties are exhibited; and the like.

By way of yet further illustration, some of suitable positively magnetized species are listed in the “CRC Handbook of Chemistry and Physics,” 63^{rd} Edition (CRC Press, Inc., Boca-Raton, Fla., 1982-1983). As is discussed on pages E-118 to E-123 of such CRC Handbook, materials with positive susceptibility include, e.g., aluminum, americium, cerium (beta form), cerium (gamma form), cesium, compounds of cobalt, dysprosium, compounds of dysprosium, europium, compounds of europium, gadolium, cmpounds of gadolinium, hafnium, compounds of holmium, iridium, compounds of iron, lithium, magnesium, manganese, molybdenum, neodymium, niobium, osmium, palladium, plutonium, potassium, praseodymium, rhodium, rubidium, ruthenium, samarium, sodium, strontium, tantalum, technicium, terbium, thorium, thulium, titanium, tungsten, uranium, vanadium, ytterbium, yttrium, and the like.

By way of comparison, and referring again to FIG. 11A, plot 705 of the negatively magnetized species is shown with a negative slope. The negatively magnetized species include those materials with negative susceptibilities that are listed on such pages E-118 to E-123 of the CRC Handbook. By way of illustration and not limitation, such species include, e.g.: antimony; argon; arsenic; barium; beryllium; bismuth; boron; calcium; carbon (dia); chromium; copper; gallium; germanium; gold; indium; krypton; lead; mercury; phosphorous; selenium; silicon; silver; sulfur; tellurium; thallium; tin (gray); xenon; zinc; and the link.

Many diamagnetic materials also are suitable negatively magnetized species. As is kown to those skilled in the art, diamagnetism is that property of a material that is repelled by magnets. The term “diamagnetic susceptibility” refers to the susceptibility of a diamagnetic material, which is always negative. Diamagnetic materials are well known to those skilled in the art. Reference may be had, e.g., to U.S. Pat. No. 6,162,364 (diamagnetic objects); U.S. Pat. No. 6,159,271 (diamagnetic liquid); U.S. Pat. No. 5,408,178 (diamagnetic and paramagnetic objects); U.S. Pat. No. 5,315,997 (method of magnetic resonance imaging using diamagnetic contrast); U.S. Pat. Nos. 5,162,301; 5,047,392 (diamagnetic colloids); U.S. Pat. No. 5,043,101; 5,026,681 (diamagnetic colloid pumps); U.S. Pat. No. 4,908,347 (diamagnetic flux shield); U.S. Pat. Nos. 4,778,594; 4,735,796; 4,590,922; 4,290,070; 3,899,758; 3,864,824; 3,815,963 (pseudo-diamagnetic suspension); U.S. Pat. Nos. 3,597,022; 3,572,273; and the like. The entire disclosure of each of these U.S. patents is hereby incorporated by reference into this specification.

By way of further illustration, the diamagnetic material used may be an organic compound with a negative suspceptibility. Referring to pages E-123 to pages E-134 of the aforementioned CRC Handbook, such compounds include, e.g.: alanine; allyl alcohol; amylamine; aniline; asparagines; aspartic acid; butyl alcohol; chloresterol; coumarin; diethylamine; erythritol; eucalyptol; fructose; galactose; glucose; D-glucose; glutamic acid; glycerol; glycine; leucine; isoleucine; mannitol; mannose; and the like.

Referring again to FIG. 11A, when a positively magnetized species is mixed with a negatively magnetized species, and assuming that each species retains its magnetic properties, the resulting magnetic properties are indicated by plot 707, with substantially zero magnetization. In this embodiment, one must insure that the positively magnetized species does not lose its magnetic properties, as often happens when one material is alloyed with another. The magnetic properties of alloys and compounds containing different species are known, and thus it readily ascertainable whether the different species that make up such alloys and/or compounds have retained their unqiue magnetic characteristics.

Without wishing to be bound to any particular theory, applicants belive that, when a positively magnetized species is mixed with a negatively magnetized species, and assuming that each species retains its magnetic properties, the plot 707 (zero magnetization) will be achieved when the volume of the positively magnetized speicies times its positive susceptibility is substantially equal to the volume of the negatively magnetized speices times its netative susceptibility For this relationship to hold, however, each of the positively magnetized species and the negatively magnetized species must retain the distinctive magnetic characteristics when mixed with each other.

Thus, for example, if element A has a positive magnetic suspceptibility, and element B has a negative magnetic suspceptibility, the alloying of A and B in equal proportions may not yield a zero magnetization compact.

Without wishing to be bound to any particular theory, nano-sized particles, or micro-sized particles (with a size of at least about 0.5 nanometers) tend to retain their magnetic properties as long as they remain in particulate form. On the other hand, alloys of such materials often do not retain such properties.

With regard to reactance (see FIG. 12) the r.f. field and the gradient field are treated as a radiation source which is applied to a living organism comprised of a stent in contact with biological material. The stent, with or without a coating, reacts to the radiation source by exhibiting a certain inductive reactance and a certain capacitative reactance. The net reactance is the difference between the inductive reactance and the capacitative reactance; and it desired that the net reactance be as close to zero as is possible. When the net reactance is greater than zero, it distorts some of the applied MRI fields and thus interferes with their imaging capabilities. Similarly, when the net reactance is less than zero, it also distorts some of the applied MRI fields.

Nullification of the Susceptibility Contribution Due to the Substrate

As will be apparent by reference, e.g., to FIG. 11, the copper substrate depicted therein has a negative susceptibility, the coating depicted therein has a positive suceptibility, and the coated substrate thus has a substantially zero susceptibility. As will also be apparent, some substrates (such niobium, nitinol, stainless steel, etc.) have positive susceptibilities. In such cases, and in one preferred embodiment, the coatings should preferably be chosen to have a negative susceptibility so that, under the conditions of the MRI radiation (or of any other radiation source used), the net susceptibility of the coated object is still substantially zero. As will be apparent, the contribution of each of the materials in the coating(s) is a function of the mass of such material and its magnetic susceptibility.

The magnetic susceptibilities of various substrate materials are well known. Reference may be had, e.g., to pages E-118 to E-123 of the “Handbook of Chemistry and Physics,” 63rd edition (CRC Press, Inc., Boca Raton, Fla., 1974).

Once the susceptibility of the substrate material is determined, one can use the following equation: χ_{sub}+χ_{coat}=0, wherein χ_{sub} is the susceptibility of the substrate, and χ_{coat} is the susceptibility of the coating, when each of these is present in a 1/1 ratio. As will be apparent, the aforementioned equation is used when the coating and substrate are present in a 1/1 ratio. When other ratios are used other than a 1/1 ratio, the volume percent of each component (or its mass) must be taken into consideration in accordance with the equation: (volume percent of substrate×susceptibility of the substrate)+(volume percent of coating×susceptibility of the coating)=0. One may use a comparable formula in which the weight percent of each component is substituted for the volume percent, if the susceptibility is measured in terms of the weight percent.

By way of illustration, and in one embodiment, the uncoated substrate may either comprise or consist essentially of niobium, which has a susceptibility of +195.0×10⁻⁶ centimeter-gram seconds at 298 degrees Kelvin.

In another embodiment, the substrate may contain at least 98 molar percent of niobium and less than 2 molar percent of zirconium. Zirconium has a susceptibility of −122×0×10⁻⁶ centimeter-gram seconds at 293 degrees Kelvin. As will be apparent, because of the predominance of niobium, the net susceptibility of the uncoated substrate will be positive.

The substrate may comprise Nitinol. Nitinol is a paramagnetic alloy, an, intermetallic compound of nickel and titanium; the alloy preferably contains from 50 to 60 percent of nickel, and it has a permeability value of about 1.002. The susceptibility of Nitinol is positive.

Nitinols with nickel content ranging from about 53 to 57 percent are known as “memory alloys” because of their ability to “remember” or return to a previous shape upon being heated . . . which is an alloy of nickel and titanium, in an approximate 1/1 ratio. The susceptibility of Nitinol is positive.

The substrate may comprise tantalum and/or titanium, each of which has a positive susceptibility. See, e.g., the CRC handbook cited above.

When the uncoated substrate has a positive susceptibility, the coating to be used for such a substrate should have a negative susceptibility. Referring again to said CRC handbook, it will be seen that the values of negative susceptibilities for various elements are −9.0 for beryllium, −280.1 for bismuth (s), −10.5 for bismuth (l), −6.7 for boron, −56.4 for bromine (l), −73.5 for bromine(g), −19.8 for cadmium(s), −18.0 for cadmium(l), −5.9 for carbon(dia), −6.0 for carbon (graph), −5.46 for copper(s), −6.16 for copper(l), −76.84 for germanium, −28.0 for gold(s), −34.0 for gold(l), −25.5 for indium, −88.7 for iodine(s), −23.0 for lead(s), −15.5 for lead(l), −19.5 for silver(s), −24.0 for silver(l), −15.5 for sulfur(alpha), −14.9 for sulfur(beta), −15.4 for sulfur(l), −39.5 for tellurium(s), −6.4 for tellurium(l), −37.0 for tin(gray), −31.7 for tin(gray), −4.5 for tin(l), −11.4 for zinc(s), −7.8 for zinc(l), and the like. As will be apparent, each of these values is expressed in units equal to the number in question×10⁻⁶ centimeter-gram seconds at a temperature at or about 293 degrees Kelvin. As will also be apparent, those materials which have a negative susceptibility value are often referred to as being diamagnetic.

By way of further reference, a listing of organic compounds that are diamagnetic is presented on pages E123 to E134 of the aforementioned “Handbook of Chemistry and Physics,” 63rd edition (CRC Press, Inc., Boca Raton, Fla., 1974).

In one embodiment, and referring again to the aforementioned “Handbook of Chemistry and Physics,” 63rd edition (CRC Press, Inc., Boca Raton, Fla., 1974), one or more of the following magnetic materials described below are preferably incorporated into the coating.

The desired magnetic materials, in this embodiment, preferably have a positive susceptibility, with values ranging from +1×10⁻⁶ centimeter-gram seconds at a temperature at or about 293 degrees Kelvin, to about 1×10⁷ centimeter-gram seconds at a temperature at or about 293 degrees Kelvin.

Thus, by way of illustration and not limitation, one may use materials such as Alnicol (see page E-112 of the CRC handbook), which is an alloy containing nickel, aluminum, and other elements such as, e.g., cobalt and/or iron. Thus, e.g., one my use silicon iron (see page E113 of the CRC handbook), which is an acid resistant iron containing a high percentage of silicon. Thus, e.g., one may use steel (see page 117 of the CRC handbook). Thus, e.g., one may use elements such as dyprosium, erbium, europium, gadolinium, hafnium, holmium, manganese, molybdenum, neodymium, nickel-cobalt, alloys of the above, and compounds of the above such as, e.g., their oxides, nitrides, carbonates, and the like.

Referring to FIG. 12, and to the embodiment depicted therein, it will be seen that the uncoated stent has an effective inductive reactance at a d.c. field of 1.5 Tesla that exceeds its capacitative reactance, whereas the coating 704 has a capacitative reatance that exceeds its inductive reactance. The coated (composite) stent 706 has a net reactance that is substantially zero.

As will be apparent, the effective inductive reactance of the uncoated stent 702 may be due to a multiplicity of factors including, e.g., the positive magnetic susceptibility of the materials which it is comprised of it, the loop currents produced, the surface eddy produced, etc. Regardless of the source(s) of its effective inductive reactance, it can be “corrected” by the use of one or more coatings which provide, in combination, an effective capacitative reactance that is equal to the effective inductive reactance.

Referring again to FIG. 9, and in the embodiment depicted, plaque particles 430,432 are disposed on the inside of substrate 404. When the net reactance of the coated substrate 404 is essentially zero, the imaging field 440 can pass substantially unimpeded through the coating 402 and the sustrate 404 and interact with the plaque particles 430/432 to produce imaging signals 441.

The imaging signals 441 are able to pass back through the substrate 404 and the coating 402 because the net reactance is substantially zero. Thus, these imaging signals are able to be received and processed by the MRI apparatus.

Thus, by the use of applicants' technology, one may negate the negative substrate effect and, additionally, provide pathways for the image signals to interact with the desired object to be imaged (such as, e.g., the plaque particles) and to produce imaging signals that are capable of escaping the substrate assembly and being received by the MRI apparatus.

Incorporation of Disclosure of U.S. Ser. No. 10/303/264, filed on Nov. 25, 2002

Applicants' hereby incorporate by reference into this specification the entire disclosure of their copending U.S. patent application Ser. No. 10/303,264, filed on Nov. 25, 2002, and also the corresponding disclosure of their U.S. Pat. No. 6,713,671, issued on Mar. 30, 2004.

U.S. patent application Ser. No. 10/303,264 (and also U.S. Pat. No. 6,713,671) discloses a shielded assembly comprised of a substrate and, disposed above a substrate, a shield comprising from about 1 to about 99 weight percent of a first nanomagnetic material, and from about 99 to about 1 weight percent of a second material with a resistivity of from about 1 microohm-centimeter to about 1×1025 microohm centimeters; the nanomagnetic material comprises nanomagnetic particles, and these nanomagnetic particles respond to an externally applied magnetic field by realigning to the externally applied field. Such a shielded assembly and/or the substrte thereof and/or the shield thereof may be used in the processes, compositions, and/or constructs of this invention.

As is disclosed in U.S. Pat. No. 6,713,617, the entire disclosoure of which is hereby incorporated by reference into this specification, in one embodiment the substrate used may be, e.g, comprised of one or more conductive material(s) that have a resistivity at 20 degrees Centigrade of from about 1 to about 100 microohm-centimeters. Thus, e.g., the conductive material(s) may be silver, copper, aluminum, alloys thereof, mixtures thereof, and the like.

In one embodiment, the substrate consists consist essentially of such conductive material. Thus, e.g., it is preferred not to use, e.g., copper wire coated with enamel in this embodiment..

In the first step of the process preferably used to make this embodiment of the invention, (see step 40 of FIG. 1 of U.S. Pat. No. 6,713,671), conductive wires are coated with electrically insulative material. Suitable insulative materials include nano-sized silicon dioxide, aluminum oxide, cerium oxide, yttrium-stabilized zirconia, silicon carbide, silicon nitride, aluminum nitride, and the like. In general, these nano-sized particles will have a particle size distribution such that at least about 90 weight percent of the particles have a maximum dimension in the range of from about 10 to about 100 nanometers.

In such process, the coated conductors may be prepared by conventional means such as, e.g., the process described in U.S. Pat. No. 5,540,959, the entire disclosure of which is hereby incorporated by reference into this specification. Alternatively, one may coat the conductors by means of the processes disclosed in a text by D. Satas on “Coatings Technology Handbook” (Marcel Dekker, Inc., New York, N.Y., 1991). As is disclosed in such text, one may use cathodic arc plasma deposition (see pages 229 et seq.), chemical vapor deposition (see pages 257 et seq.), sol-gel coatings (see pages 655 et seq.), and the like.

FIG. 2 of U.S. Pat. No. 6,713,671 is a sectional view of the coated conductors 14/16. In the embodiment depicted in such FIG. 2, itt will be seen that conductors 14 and 16 are separated by insulating material 42. In order to obtain the structure depicted in such FIG. 2, one may simultaneously coat conductors 14 and 16 with the insulating material so that such insulators both coat the conductors 14 and 16 and fill in the distance between them with insulation.

Referring again to such FIG. 2 of U.S. Pat. No. 6,713,671, the insulating material 42 that is disposed between conductors 14/16, may be the same as the insulating material 44/46 that is disposed above conductor 14 and below conductor 16. Alternatively, and as dictated by the choice of processing steps and materials, the insulating material 42 may be different from the insulating material 44 and/or the insulating material 46. Thus, step 48 of the process of such FIG. 2 describes disposing insulating material between the coated conductors 14 and 16. This step may be done simultaneously with step 40; and it may be done thereafter.

Referring again to such FIG. 2, and to the preferred embodiment depicted therein, the insulating material 42, the insulating material 44, and the insulating material 46 each generally has a resistivity of from about 1,000,000,000 to about 10,000,000,000,000 ohm-centimeters.

Referring again to FIG. 2 of U.S. Pat. No. 6,713,671, after the insulating material 42/44/46 has been deposited, and in one embodiment, the coated conductor assembly is preferably heat treated in step 50. This heat treatment often is used in conjunction with coating processes in which the heat is required to bond the insulative material to the conductors 14/16.

The heat-treatment step may be conducted after the deposition of the insulating material 42/44/46, or it may be conducted simultaneously therewith. In either event, and when it is used, it is preferred to heat the coated conductors 14/16 to a temperature of from about 200 to about 600 degrees Centigrade for from about 1 minute to about 10 minutes.

Referring again to FIG. 1A of U.S. Pat. No. 6,713,67, and in step 52 of the process, after the coated conductors 14/16 have been subjected to heat treatment step 50, they are allowed to cool to a temperature of from about 30 to about 100 degrees Centigrade over a period of time of from about 3 to about 15 minutes.

One need not invariably heat treat and/or cool. Thus, referring to such FIG. 1A, one may immediately coat nanomagnetic particles onto to the coated conductors 14/16 in step 54 either after step 48 and/or after step 50 and/or after step 52.

Referring again to FIG. 1A of U.S. Pat. No. 6,713,67, in step 54, nanomagnetic materials are coated onto the previously coated conductors 14 and 16. This is best shown in FIG. 2 of such patent, wherein the nanomagnetic particles are identified as particles 24.

In general, and as is known to those skilled in the art, nanomagnetic material is magnetic material which has an average particle size less than 100 nanometers and, preferably, in the range of from about 2 to 50 nanometers. Reference may be had, e.g., to U.S. Pat. No. 5,889,091 (rotationally free nanomagnetic material), U.S. Pat. Nos. 5,714,136, 5,667,924, and the like. The entire disclosure of each of these U.S. patents is hereby incorporated by reference into this specification.

In general, the thickness of the layer of nanomagnetic material deposited onto the coated conductors 14/16 is less than about 5 microns and generally from about 0.1 to about 3 microns.

Referring again to FIG. 2 of U.S. Pat. No. 6,713,671, after the nanomagnetic material is coated in step 54, the coated assembly may be optionally heat-treated in step 56. In this optional step 56, it is preferred to subject the coated conductors 14/16 to a temperature of from about 200 to about 600 degrees Centigrade for from about 1 to about 10 minutes.

In one embodiment, illustrated in FIG. 3 of U.S. Pat. No. 6,713,671, one or more additional insulating layers 43 are coated onto the assembly depicted in FIG. 2 of such patent. This is conducted in optional step 58 (see FIG. 1A of such patent).

FIG. 4 of U.S. Pat. No. 6,713,671 is a partial schematic view of the assembly 11 of FIG. 2 of such patent, illustrating the current flow in such assembly. Referring again to FIG. 4 of U.S. Pat. No. 6,713,671, it will be seen that current flows into conductor 14 in the direction of arrow 60, and it flows out of conductor 16 in the direction of arrow 62. The net current flow through the assembly 11 is zero; and the net Lorentz force in the assembly 11 is thus zero. Consequently, even high current flows in the assembly 11 do not cause such assembly to move.

Referring again to FIG. 4 of U.S. Pat. No. 6,713,67. conductors 14 and 16 are substantially parallel to each other. As will be apparent, without such parallel orientation, there may be some net current and some net Lorentz effect.

In the embodiment depicted in such FIG. 4, and in one preferred aspect thereof, the conductors 14 and 16 preferably have the same diameters and/or the same compositions and/or the same length.

Referring again to FIG. 4 of U.S. Pat. No. 6,713,671, the nanomagnetic particles 24 are present in a density sufficient so as to provide shielding from magnetic flux lines 64. Without wishing to be bound to any particular theory, applicant believes that the nanomagnetic particles 24 trap and pin the magnetic lines of flux 64.

In order to function optimally, the nanomagnetic particles 24 preferably have a specified magnetization. As is known to those skilled in the art, magnetization is the magnetic moment per unit volume of a substance. Reference may be had, e.g., to U.S. Pat. Nos. 4,169,998, 4,168,481, 4,166,263, 5,260,132, 4,778,714, and the like. The entire disclosure of each of these U.S. patents is hereby incorporated by reference into this specification.

Referring again to FIG. 4 of U.S. Pat. No. 6,713,671, the entire disclosure of which is hereby incorporated by reference into this specification, the layer of nanomagnetic particles 24 preferably has a saturation magnetization, at 25 degrees Centigrade, of from about 1 to about 36,000 Gauss, or higher. In one embodiment, the saturation magnetization at room temperature of the nanomagentic particles is from about 500 to about 10,000 Gauss. For a discussion of the saturation magnetization of various materials, reference may be had, e.g., to U.S. Pat. Nos. 4,705,613, 4,631,613, 5,543,070, 3,901,741 (cobalt, samarium, and gadolinium alloys), and the like. The entire disclosure of each of these U.S. patents is hereby incorporated by reference into this specification.

In one embodiment, it is preferred to utilize a thin film with a thickness of less than about 2 microns and a saturation magnetization in excess of 20,000 Gauss. The thickness of the layer of nanomagentic material is measured from the bottom surface of the layer that contains such material to the top surface of such layer that contains such material; and such bottom surface and/or such top surface may be contiguous with other layers of material (such as insulating material) that do not contain nanomagnetic particles.

Thus, e.g., one may make a thin film in accordance with the procedure described at page 156 of Nature, Volume 407, Sep. 14, 2000, that describes a multilayer thin film has a saturation magnetization of 24,000 Gauss.

Referring again to FIG. 4 of U.S. Pat. No. 6,713,671, the nanomagnetic particles 24 are disposed within an insulating matrix so that any heat produced by such particles will be slowly dispersed within such matrix. Such matrix, as indicated hereinabove, may be made from ceria, calcium oxide, silica, alumina. In general, the insulating material 42 preferably has a thermal conductivity of less than about 20 (caloriescentimeters/square centimeters-egree second)×10,000. See, e.g., page E-6 of the 63rd Edition of the “Handbook of Chemistry and Physics” (CRC Press, Inc., Boca Raton, Fla., 1982).

The nanomagnetic materials 24 typically comprise one or more of iron, cobalt, nickel, gadolinium, and samarium atoms. Thus, e.g., typical nanomagnetic materials include alloys of iron and nickel (permalloy), cobalt, niobium, and zirconium (CNZ), iron, boron, and nitrogen, cobalt, iron, boron, and silica, iron, cobalt, boron, and fluoride, and the like. These and other materials are described in a book by J. Douglas Adam et al. entitled “Handbook of Thin Film Devices” (Academic Press, San Diego, Calif., 2000). Chapter 5 of this book beginning at page 185, describes “magnetic films for planar inductive components and devices;” and Tables 5.1 and 5.2 in this chapter describe many magnetic materials.

FIG. 5 of U.S. Pat. No. 6,713,671 is a sectional view of the assembly 11 of FIG. 2 of such patent. The device of such FIG. 5 is preferably substantially flexible. As used in this specification, the term flexible refers to an assembly that can be bent to form a circle with a radius of less than 2 centimeters without breaking. Put another way, the bend radius of the coated assembly 11 can be less than 2 centimeters. Reference may be had, e.g., to U.S. Pat. Nos. 4,705,353, 5,946,439, 5,315,365, 4,641,917, 5,913,005, and the like. The entire disclosure of each of these U.S. patents is hereby incorporated by reference into this specification.

In another embodiment, not shown, the shield is not flexible. Thus, in one aspect o f this embodiment, the shield is a rigid, removable sheath that can be placed over an endoscope or a biopsy probe used inter-operatively with magnetic resonance imaging.

In another embodiment of the invention of U.S. Pat. No. 6,713,671, there is provided a magnetically shielded conductor assembly comprised of a conductor and a film of nanomagnetic material disposed above said conductor. In this embodiment, the conductor has a resistivity at 20 degrees Centigrade of from about 1 to about 2,000 micro ohm-centimeters and is comprised of a first surface exposed to electromagnetic radiation. In this embodiment, the film of nanomagnetic material has a thickness of from about 100 nanometers to about 10 micrometers and a mass density of at least about 1 gram per cubic centimeter, wherein the film of nanomagnetic material is disposed above at least about 50 percent of said first surface exposed to electromagnetic radiation, and the film of nanomagnetic material has a saturation magnetization of from about 1 to about 36,000 Gauss, a coercive force of from about 0.01to about 5,000 Oersteds, a relative magnetic permeability of from about 1 to about 500,000, and a magnetic shielding factor of at least about 0.5. In this embodiment, the nanomagnetic material has an average particle size of less than about 100 nanometers.

In one preferred embodiment of this invention, and referring to FIG. 6 of U.S. Pat. No. 6,713,671, a film of nanomagnetic material is disposed above at least one surface of a conductor. Referring to such FIG. 6, and in the schematic diagram depicted therein, a source of electromagnetic radiation 100 emits radiation 102 in the direction of film 104. Film 104 is disposed above conductor 106, i.e., it is disposed between conductor 106 of the electromagnetic radiation 102.

Referring again to FIG. 6 of U.S. Pat. No. 6,713,671, the film 104 is adapted to reduce the magnetic field strength at point 108 (which is disposed less than 1 centimeter above film 104) by at least about 50 percent. Thus, if one were to measure the magnetic field strength at point 108, and thereafter measure the magnetic field strength at point 110 (which is disposed less than 1 centimeter below film 104), the latter magnetic field strength would be no more than about 50 percent of the former magnetic field strength. Put another way, the film 104 has a magnetic shielding factor of at least about 0.5.

Referring again to FIG. 6 of U.S. Pat. No. 6,713,671, in one embodiment, the film 104 has a magnetic shielding factor of at least about 0.9, i.e., the magnetic field strength at point 110 is no greater than about 10 percent of the magnetic field strength at point 108. Thus, e.g., the static magnetic field strength at point 108 can be, e.g., one Tesla, whereas the static magnetic field strength at point 110 can be, e.g., 0.1 Tesla. Furthermore, the time-varying magnetic field strength of a 100 milliTesla would be reduced to about 10 milliTesla of the time-varying field.

Referring again to FIG. 6 of U.S. Pat. No. 6,713,671, the nanomagnetic material 103 in film 104 has a saturation magnetization of form about 1 to about 36,000 Gauss. In one embodiment, the nanomagnetic material 103 a saturation magnetization of from about 200 to about 26,000 Gauss.

Referring again to FIG. 6 of U.S. Pat. No. 6,713,671, the nanomagnetic material 103 in film 104 also has a coercive force of from about 0.01 to about 5,000 Oersteds. The term coercive force refers to the magnetic field, H, which must be applied to a magnetic material in a symmetrical, cyclicly magnetized fashion, to make the magnetic induction, B, vanish; this term often is referred to as magnetic coercive force. Reference may be had, e.g., to U.S. Pat. Nos. 4,061,824, 6,257,512, 5,967,223, 4,939,610, 4,741,953, and the like. The entire disclosure of each of these U.S. patents is hereby incorporated by reference into this specification.

Referring again to FIG. 6 of U.S. Pat. No. 6,713,671, in one embodiment, the nanomagnetic material 103 has a coercive force of from about 0.01 to about 3,000 Oersteds. In yet another embodiment, the nanomagnetic material 103 has a coercive force of from about 0.1 to about 10.

Referring again to such FIG. 6, the nanomagnetic material 103 in film 104 preferably has a relative magnetic permeability of from about 1 to about 500,000; in one embodiment, such material 103 has a relative magnetic permeability of from about 1.5 to about 260,000. As used in this specification, the term relative magnetic permeability is equal to B/H, and is also equal to the slope of a section of the magnetization curve of the film. Reference may be had, e.g., to page 4-28 of E. U. Condon et al.'s “Handbook of Physics” (McGraw-Hill Book Company, Inc., New York, 1958).

Reference also may be had to page 1399 of Sybil P. Parker's “McGraw-Hill Dictionrary of Scientific and Technical Terms,” Fourth Edition (McGraw Hill Book Company, New York, 1989). As is disclosed on this page 1399, permeability is “. . . a factor, characteristic of a material, that is proportional to the magnetic induction produced in a material divided by the magnetic field strength; it is a tensor when these quantities are not parallel.”

Reference also may be had, e.g., to U.S. Pat. Nos. 6,181,232, 5,581,224, 5,506,559, 4,246,586, 6,390,443, and the like. The entire disclosure of each of these U.S. patents is hereby incorporated by reference into this specification.

In one embodiment, the nanomagnetic material 103 in film 104 has a relative magnetic permeability of from about 1.5 to about 2,000.

Referring again to FIG. 6 of U.S. Pat. No. 6,713,671, the nanomagnetic material 103 in film 104 preferably has a mass density of at least about 0.001 grams per cubic centimeter; in one embodiment, such mass density is at least about 1 gram per cubic centimeter. As used in this specification, the term mass density refers to the mass of a give substance per unit volume. See, e.g., page 510 of the aforementioned “McGraw-Hill Dictionary of Scientific and Technical Terms.” In one embodiment, the film 104 has a mass density of at least about 3 grams per cubic centimeter. In another embodiment, the nanomagnetic material 103 has a mass density of at least about 4 grams per cubic centimeter.

Referring again to FIG. 6 of U.S. Pat. No. 6,713,671, and in the embodiment depicted in such FIG. 6, the film 104 is disposed above 100 percent of the surfaces 112, 114, 116, and 118 of the conductor 106. In the embodiment depicted in FIG. 2, by comparison, the nanomagnetic film is disposed around the conductor.

Yet another embodiment is depicted in FIG. 7 of U.S. Pat. No. 6,713,671 In the embodiment depicted in FIG. 7, the film 104 is not disposed in front of either surface 114, or 116, or 118 of the conductor 106. Inasmuch as radiation is not directed towards these surfaces, this is possible.

What is essential, however, is that the film 104 be interposed between the radiation 102 and surface 112. It is preferred that film 104 be disposed above at least about 50 percent of surface 112. In one embodiment, film 104 is disposed above at least about 90 percent of surface 112.

Referring again to FIG. 8A of U.S. Pat. No. 6,713,671, and in the preferred embodiment depicted in FIG. 8A, the nanomagnetic material 202 may be disposed within an insulating matrix (not shown) so that any heat produced by such particles will be slowly dispersed within such matrix. Such matrix, as indicated hereinabove, may be made from ceria, calcium oxide, silica, alumina, and the like. In general, the insulating material 202 preferably has a thermal conductivity of less than about 20 (calories centimeters/square centimeters-degree second)×10,000. See, e.g., page E-6 of the 63rd. Edition of the “Handbook of Chemistry and Physics” (CRC Press, Inc. Boca Raton, Fla., 1982).

Referring again to FIG. 8A of U.S. Pat. No. 6,713,67, and in the preferred embodiment depicted therein the nanomagnetic material 202 typically comprises one or more of iron, cobalt, nickel, gadolinium, and samarium atoms. Thus, e.g., typical nanomagnetic materials include alloys of iron, and nickel (permalloy), cobalt, niobium and zirconium (CNZ), iron, boron, and nitrogen, cobalt, iron, boron and silica, iron, cobalt, boron, and fluoride, and the like. These and other materials are described in a book by J. Douglass Adam et al. entitled “Handbook of Thin Film Devices” (Academic Press, San Diego, Calif., 2000). Chapter 5 of this book beginning at page 185 describes “magnetic films for planar inductive components and devices;” and Tables 5.1.and 5.2 in this chapter describes many magnetic materials.

FIG. 11 of U.S. Pat. No. 6,713,671 is a schematic sectional view of a substrate 401, which is part of an implantable medical device (not shown). Referring to such FIG. 11, and in the preferred embodiment depicted therein, it will be seen that substrate 401 is coated with a layer 404 of nanomagnetic material(s). The layer 404, in the embodiment depicted, is comprised of nanomagnetic particulate 405 and nanomagnetic particulate 406. Each of the nanomagnetic particulate 405 and nanomagnetic particulate 406 preferably has an elongated shape, with a length that is greater than its diameter. In one aspect of this embodiment, nanomagnetic particles 405 have a different size than nanomagnetic particles 406. In another aspect of this embodiment, nanomagnetic particles 405 have different magnetic properties than nanomagnetic particles 406. Referring again to such FIG. 11, and in the preferred embodiment depicted therein, nanomagnetic particulate material 405 and nanomagnetic particulate material 406 are designed to respond to an static or time-varying electromagnetic fields or effects in a manner similar to that of liquid crystal display (LCD) materials. More specifically, these nanomagnetic particulate materials 405 and nanomagnetic particulate materials 406 are designed to shift alignment and to effect switching from a magnetic shielding orientation to a non-magnetic shielding orientation. As will be apparent, the magnetic shield provided by layer 404, can be turned “On” and “OFF” upon demand. In yet another embodiment (not shown), the magnetic shield is turned on when heating of the shielded object is detected.

In one embodiment of the invention, also described in U.S. Pat. No. 6,713,671, there is provided a coating of nanomagnetic particles that consists of a mixture of aluminum oxide (Al2O3), iron, and other particles that have the ability to deflect electromagnetic fields while remaining electrically non-conductive. Preferably the particle size in such a coating is approximately 10 nanometers. Preferably the particle packing density is relatively low so as to minimize electrical conductivity. Such a coating when placed on a fully or partially metallic object (such as a guide wire, catheter, stent, and the like) is capable of deflecting electromagnetic fields, thereby protecting sensitive internal components, while also preventing the formation of eddy currents in the metallic object or coating. The absence of eddy currents in a metallic medical device provides several advantages, to wit: (1) reduction or elimination of heating, (2) reduction or elimination of electrical voltages which can damage the device and/or inappropriately stimulate internal tissues and organs, and (3) reduction or elimination of disruption and distortion of a magnetic-resonance image.

In one portion of U.S. Pat. No. 6,713,671, the patentees described one embodiment of a composite shield. This embodiment involves a shielded assembly comprised of a substrate and, disposed above a substrate, a shield comprising from about 1 to about 99 weight percent of a first nanomagnetic material, and from about 99 to about 1 weight percent of a second material with a resistivity of from about 1 microohm-centimeter to about 1×1025 microohm centimeters.

FIG. 29 of U.S. Pat. No. 6,713,671 is a schematic of a preferred shielded assembly 3000 that is comprised of a substrate 3002. The substrate 3002 may be any one of the substrates illustrated hereinabove. Alternatively, or additionally, it may be any receiving surface which it is desired to shield from magnetic and/or electrical fields. Thus, e.g., the substrate can be substantially any size, any shape, any material, or any combination-of materials. The shielding material(s) disposed on and/or in such substrate may be disposed on and/or in some or all of such substrate.

Referring again to FIG. 29 of U.S. Pat. No. 6,713,671, and by way of illustration and not limitation, the substrate 3002 may be, e.g., a foil comprised of metallic material and/or polymeric material. The substrate 3002 may, e.g., comprise ceramic material, glass material, composites, etc. The substrate 3002 may be in the shape of a cylinder, a sphere, a wire, a rectilinear shaped device (such as a box), an irregularly shaped device, etc.

Referring again to FIG. 29 of U.S. Pat. No. 6,713,67, and in one embodiment, the substrate 3002 preferably a thickness of from about 100 nanometers to about 2 centimeters. In one aspect of this embodiment, the substrate 3002 preferably is flexible.

Referring again to FIG. 29 of U.S. Pat. No. 6,713,671, and in the preferred embodiment depicted therein, it will be seen that a shield 3004 is disposed above the substrate 3002. As used herein, the term “above” refers to a shield that is disposed between a source 3006 of electromagnetic radiation and the substrate 3002.

The shield 3004 is comprised of from about 1 to about 99 weight percent of nanomagnetic material 3008; such nanomagnetic material, and its properties, are described elsewhere in this specification. In one embodiment, the shield 3004 is comprised of at least about 40 weight percent of such nanomagnetic material 3008. In another embodiment, the shield 3004 is comprised of at least about 50 weight percent of such nanomagnetic material 3008.

Referring again to FIG. 29 of such U.S. Pat. No. 6,713,671, and in the preferred embodiment depicted therein, it will be seen that the shield 3004 is also comprised of another material 3010 that preferably has an electrical resistivity of from about about 1 microohm-centimeter to about 1×1025 microohm-centimeters. This material 3010 is preferably present in the shield at a concentration of from about 1 to about 1 to about 99 weight percent and, more preferably, from about 40 to about 60 weight percent.

In one embodiment, the material 3010 has a dielectric constant of from about 1 to about 50 and, more preferably, from about 1.1 to about 10. In another embodiment, the material 3010 has resistivity of from about 3 to about 20 microohm-centimeters.

In one embodiment, the material 3010 preferably is a nanoelectrical material with a particle size of from about 5 nanometers to about 100 nanometers.

In another embodiment, the material 3010 has an elongated shape with an aspect ratio (its length divided by its width) of at least about 10. In one aspect of this embodiment, the material 3010 is comprised of a multiplicity of aligned filaments.

In one embodiment, the material 3010 is comprised of one or more of the compositions of U.S. Pat. Nos. 5,827,997 and 5,643,670.

Thus, e.g., the material 3010may comprise filaments, wherein each filament comprises a metal and an essentially coaxial core, each filament having a diameter less than about 6 microns, each core comprising essentially carbon, such that the incorporation of 7 percent volume of this material in a matrix that is incapable of electromagnetic interference shielding results in a composite that is substantially equal to copper in electromagnetic interference shielding effectives at 1-2 gigahertz. Reference may be had, e.g., to U.S. Pat. No. 5,827,997, the entire disclosure of which is hereby incorporated by reference into this specification.

In another embodiment, the material 3010 is a particulate carbon complex comprising: a carbon black substrate, and a plurality of carbon filaments each having a first end attached to said carbon black substrate and a second end distal from said carbon black substrate, wherein said particulate carbon complex transfers electrical current at a density of 7000 to 8000 milliamperes per square centimeter for a Fe+2/Fe+3 oxidation/reduction electrochemical reaction couple carried out in an aqueous electrolyte solution containing 6 millmoles of potassium ferrocyanide and one mole of aqueous potassium nitrate.

In another embodiment, the material 3010 may be a diamond-like carbon material. As is known to those skilled in the art, this diamond-like carbon material has a Mohs hardness of from about 2 to about 15 and, preferably, from about 5 to about 15. Reference may be had, e.g., to U.S. Pat. No. 5,098,737 (amorphic diamond material), U.S. Pat. No. 5,658,470 (diamond-like carbon for ion milling magnetic material), U.S. Pat. No. 5,731,045 (application of diamond-like carbon coatings to tungsten carbide components), U.S. Pat. No. 6,037,016 (capacitively coupled radio frequency diamond-like carbon reactor), U.S. Pat. No. 6,087,025 (application of diamond like material to cutting surfaces), and the like. The entire disclosure of each of these U.S. patents is hereby incorporated by reference into this specification.

In another embodiment, material 3010 is a carbon nanotube material. These carbon nanotubes generally have a cylindrical shape with a diameter of from about 2 nanometers to about 100 nanometers, and length of from about 1 micron to about 100 microns.

These carbon nanotubes are well known to those skilled in the art. Reference may be had, e.g., to U.S. Pat. No. 6,203,864 (heterojunction comprised of a carbon nanotube), U.S. Pat. No. 6,361,861 (carbon nanotubes on a substrate), U.S. Pat. No. 6,445,006 (microelectronic device comprising carbon nanotube components), U.S. Pat. No. 6,457,350 (carbon nanotube probe tip), and the like. The entire disclosure of each of these U.S. patents is hereby incorporated by reference into this specification.

In one embodiment, material 3010 is silicon dioxide particulate matter with a particle size of from about 10 nanometers to about 100 nanometers.

In another embodiment, the material 3010 is particulate alumina, with a particle size of from about 10 to about 100 nanometers. Alternatively, or additionally, one may use aluminum nitride particles, cerium oxide particles, yttrium oxide particles, combinations thereof, and the like; regardless of the particle(s) used, it is preferred that its particle size be from about 10 to about100 nanometers.

Referring again to FIG. 29 of U.S. Pat. No. 6,713,671, and in the embodiment depicted in such FIG. 29, the shield 3004 is in the form of a layer of material that has a thickness of from about 100 nanometers to about 10 microns. In this embodiment, both the nanomagnentic particles 3008 and the electrical particles 3010 are present in the same layer.

In the embodiment depicted in FIG. 30 of U.S. Pat. No. 6,713,671, by comparison, the shield 3012 is comprised of layers 3014 and 3016. The layer 3014 is comprised of at least about 50 weight percent of nanomagnetic material 3008 and, preferably, at least about 90 weight percent of such nanomagnetic material 3008. The layer 3016 is comprised of at least about 50 weight percent of electrical material 3010 and, preferably, at least about 90 weight percent of such electrical material 3010.

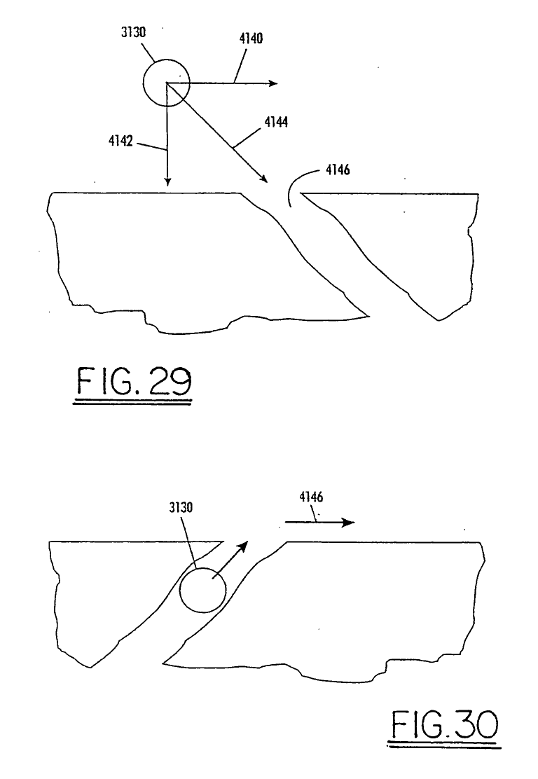

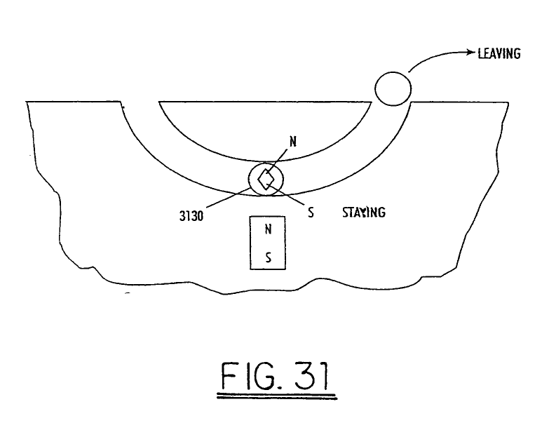

Referring to FIG. 30 of U.S. Pat. No. 6,713,671, the entire disclosure of which is hereby incorporated by reference into this specification, and in the embodiment depicted therein, the layer 3014 is disposed between the substrate 3002 and the layer 3016. In the embodiment depicted in FIG. 31, the layer 3016 is disposed between the substrate 3002 and the layer 3014. Each of the layers 3014 and 3016 preferably has a thickness of from about 10 nanometers to about 5 microns.

Referring again to FIG. 30 of U.S. Pat. No. 6,713,671, and in one embodiment, the shield 3012 has an electromagnetic shielding factor of at least about 0.9., i.e., the electromagnetic field strength at point 3020 is no greater than about 10 percent of the electromagnetic field strength at point 3022.

Referring again to FIG. 31 of U.S. Pat. No. 6,713,671, and in one preferred embodiment, the nanomagnetic material preferably has a mass density of at least about 0.01 grams per cubic centimeter, a saturation magnetization of from about 1 to about 36,000 Gauss, a coercive force of from about 0.01 to about 5000 Oersteds, a relative magnetic permeability of from about 1 to about 500,000, and an average particle size of less than about 100 nanometers.

In one embodiment, the medical devices described elsewhere in this specification are coated with a coating that provides specified “signature” when subjected to the MRI field, regardless of the orientation of the device. Such a medical device may be the sealed container 12 (see FIG. 1), a stent, etc. For the purposes of simplicity of description, the coating of a stent will be described, it being understood that the same technology could be used to coat other medical devices. Th effect of such coating is illustrated in FIG. 13.

FIG. 13 is a plot of the image response of the MRI apparatus (image clarity) as a function of the applied MRI fields. The image clarity is generally related to the net reactance.

Referring to FIG. 13, plot 802 illustrates the response of a particular uncoated stent in a first orientation in a patient's body. As will be seen from plot 802, this stent in this first orientation has an effective net inductive response.

FIG. 13, and in particular plot 804, illustrates the response of the same uncoated stent in a second orientation in a patient's body. As has been discussed elsewhere in this specification, the response of an uncoated stent is orientation specific. Thus, plot 804 shows a smaller inductive response than plot 802.

When the uncoated stent is coated with the appropriate coating, as described elsewhere in this specification, the net reactive effect is zero, as is illustrated in plot 806. In this plot 806, the magnetic response of the substrate is nullified regardless of the orientation of such substrate within a patient's body.

In one embodiment, illustrated as plot 808, a stent is coated in such a manner that its net reactance is substantially larger than zero, to provide a unique imaging signature for such stent. Because the imaging response of such coated stent is also orientation independent, one may determine its precise location in a human body with the use of conventional MRI imaging techniques. In effect, the coating on the stent 808 acts like a tracer, enabling one to locate the position of the stent 808 at will.

In one embodiment, if one knows the MRI signature of a stent in a certain condition, one may be able to determine changes in such stent. Thus, for example, if one knows the signature of such stent with plaque deposited on it, and the signature of such stent without plaque deposited on it, one may be able to determine a human body's response to such stent.

Preparation of Coatings Comprised of Nanoelectrical Material

In this portion of the specification, coatings comprised of nanoelectrical material will be described. In accordance with one aspect of this invention, there is provided a nanoelectrical material with an average particle size of less than 100 nanometers, a surface area to volume ratio of from about 0.1 to about 0.05 l/nanometer, and a relative dielectric constant of less than about 1.5.

The nanoelectrical particles of aspect of the invention have an average particle size of less than about 100 nanometers. In one embodiment, such particles have an average particle size of less than about 50 nanometers. In yet another embodiment, such particles have an average particle size of less than about 10 nanometers.

The nanoelectrical particles of this invention have surface area to volume ratio of from about 0.1 to about 0.05 l/nanometer.

When the nanoelectrical particles of this invention are agglomerated into a cluster, or when they are deposited onto a substrate, the collection of particles preferably has a relative dielectric constant of less than about 1.5. In one embodiment, such relative dielectric constant is less than about 1.2.

In one embodiment, the nanoelectrical particles of this invention are preferably comprised of aluminum, magnesium, and nitrogen atoms. This embodiment is illustrated in FIG. 14.

FIG. 14 illustrates a phase diagram 2000 comprised of moieties A, B, and C. Moiety A is preferably selected from the group consisting of aluminum, copper, gold, silver, and mixtures thereof. It is preferred that the moiety A have a resistivity of from about 2 to about 100 microohm-centimeters. In one preferred embodiment, A is aluminum with a resistivity of about 2.824 microohm-centimeters. As will apparent, other materials with resistivities within the desired range also may be used.

Referring again to FIG. 14, C is selected from the group consisting of nitrogen and oxygen. It is preferred that C be nitrogen, and A is aluminum; and aluminum nitride is present as a phase in system.

Referring again to FIG. 14, B is preferably a dopant that is present in a minor amount in the preferred aluminum nitride. In general, less than about 50 percent (by weight) of the B moiety is present, by total weight of the doped aluminum nitride. In one aspect of this embodiment, less than about 10 weight percent of the B moiety is present, by total weight of the doped aluminum nitride.

The B moiety may be, e.g., magnesium, zinc, tin, indium, gallium, niobium, zirconium, strontium, lanthanum, tungsten, mixtures thereof, and the like. In one embodiment, B is selected from the group consisting of magnesium, zinc, tin, and indium. In another especially preferred embodiment, the B moiety is magnesium.

Referring again to FIG. 14, and when A is aluminum, B is magnesium, and C is nitrogen, it will be seen that regions 2002 and 2003 correspond to materials which have a low relative dielectric constant (less than about 1.5), and a high relative dielectric constant (greater than about 1.5), respectively.

FIG. 15 is a schematic view of a coated substrate 2004 comprised of a substrate 2005 and a multiplicity of nanoelectrical particles 2006. In this embodiment, it is preferred that the nanoelectrical particles 2006 form a film with a thickness 2007 of from about 10 nanometers to about 2 micrometers and, more preferably, from about 100 nanometers to about 1 micrometer.

A Coated Substrate With a Dense Coating

FIGS. 16A and 16B are sectional and top views, respectively, of a coated substrate 2100 assembly comprised of a substrate 2102 and, disposed therein, a coating 2104.

In the embodiment depicted, the coating 2104 has a thickness 2106 of from about 400 to about 2,000 nanometers and, in one embodiment, has a thickness of from about 600 to about 1200 nanometers.

Referring again to FIGS. 16A and 16B, it will be seen that coating 2104 has a morphological density of at least about 98 percent. As is known to those skilled in the art, the morphological density of a coating is a function of the ratio of the dense coating material on its surface to the pores on its surface; and it is usually measured by scanning electron microscopy.

By way of illustration, published U.S. patent application US 2003/0102222A1 contains a FIG. 3A that is a scanning electron microscope (SEM) image of a coating of “long” single-walled carbon nanotubes on a substrate. Referring to this SEM image, it will be seen that the white areas are the areas of the coating where pores occur.

The technique of making morphological density measurements also is described, e.g., in a M.S. thesis by Raymond Lewis entitled “Process study of the atmospheric RF plasma deposition system for oxide coatings” that was deposited in the Scholes Library of Alfred University, Alfred, New York in 1999 (call Number TP2 a75 1999 vol 1., no. 1.).

FIGS. 16A and 16B schematically illustrate the porosity of the side 2107 of coating 2104, and the top 2109 of the coating 2104. The SEM image depicted shows two pores 2108 and 2110 in the cross-sectional area 2107, and it also shows two pores 2212 and 2114 in the top 2109. As will be apparent, the SEM image can be divided into a matrix whose adjacent lines 2116/2120, and adjacent lines 2118/2122 define square portion with a surface area of 100 square nanometers (10 nanometers×10 nanometers). Each such square portion that contains a porous area is counted, as is each such square portion that contains a dense area. The ratio of dense areas/porous areas, ×100, is preferably at least 98. Put another way, the morphological density of the coating 2104 is at least 98 percent. In one embodiment, the morphological density of the coating 2104 is at least about 99 percent. In another embodiment, the morphological density of the coating 2104 is at least about 99.5 percent.

One may obtain such high morphological densities by atomic size deposition, i.e., the particles sizes deposited on the substrate are atomic scale. The atomic scale particles thus deposited often interact with each other to form nano-sized moieties that are less than 100 nanometers in size.

In one embodiment, the coating 2104 (see FIGS. 16A and 16B) has an average surface roughness of less than about 100 nanometers and, more preferably, less than about 10 nanometers. As is known to those skilled in the art, the average surface roughness of a thin film is preferably measured by an atomic force microscope (AFM). Reference may be had, e.g., to U.S. Pat. No. 5,420,796 (method of inspecting planarity of wafer surface), U.S. Pat. Nos. 6,610,004, 6,140,014, 6,548,139, 6,383,404, 6,586,322, 5,832,834, and 6,342,277. The entire disclosure of each of these U.S. patents is hereby incorporated by reference into this specification.

Alternatively, or additionally, one may measure surface roughness by a laser interference technique. This technique is well known. Reference may be had, e.g., to U.S. Pat. No. 6,285,456 (dimension measurement using both coherent and white light interferometers), U.S. Pat. Nos. 6,136,410, 5,843,232 (measuring deposit thickness), U.S. Pat. No. 4,151,654 (device for measuring axially symmetric aspherics), and the like. The entire disclosure of these U.S. patents are hereby incorporated by reference into this specification.

In one embodiment, the coated substrate of this invention has durable magnetic properties that do not vary upon extended exposure to a saline solution. If the magnetic moment of a coated substrate is measured at “time zero” (i.e., prior to the time it has been exposed to a saline solution), and then the coated substrate is then immersed in a saline solution comprised of 7.0 mole percent of sodium chloride and 93 mole percent of water, and if the substrate/saline solution is maintained at atmospheric pressure and at temperature of 98.6 degrees Fahrenheit for 6 months, the coated substrate, upon removal from the saline solution and drying, will be found to have a magnetic moment that is within plus or minus 5 percent of its magnetic moment at time zero.

In another embodiment, the coated substrate of this invention has durable mechanical properties when tested by the saline immersion test described above.

In one embodiment, the coating 2104 is biocompatible with biological organisms. As used herein, the term biocompatible refers to a coating whose chemical composition does not change substantially upon exposure to biological fluids. Thus, when the coating 2104 is immersed in a 7.0 mole percent saline solution for 6 months maintained at a temperature of 98.6 degrees Fahrenheit, its chemical composition (as measured by, e.g., energy dispersive X-ray analysis [EDS, or EDAX]) is substantially identical to its chemical composition at “time zero.”

A Preferred Process of the Invention

In one embodiment of the invention, best illustrated in FIG. 9, a coated stent is imaged by an MRI imaging process. As will be apparent to those skilled in the art, the process depicted in FIG. 9 can be used with reference to other medical devices such as, e.g., a coated brachytherapy seed (see, e.g., FIG. 1).

In the first step of this process, the coated stent described by reference to FIG. 9 is contacted with the radio-frequency, direct current, and gradient fields normally associated with MRI imaging processes; these fields are discussed elsewhere in this specification. They are depicted as an MRI imaging signal 440 in FIG. 9 In the second step of this process, the MRI imaging signal 440 penetrates the coated stent 400 and interacts with material disposed on the inside of such stent, such as, e.g., plaque particles 430 and 432. This interaction produces a signal best depicted as arrow 441 in FIG. 9.

In one embodiment, the signal 440 is substantially unaffected by its passage through the coated stent 400. Thus, in this embodiment, the radio-frequency field that is disposed on the outside of the coated stent 400 is substantially the same as the radio-frequency field that passes through and is disposed on the inside of the coated stent 400.

By comparison, when the stent (not shown) is not coated with the coatings of this invention, the characteristics of the signal 440 are substantially varied by its passage through the uncoated stent. Thus, with such uncoated stent, the radio-frequency signal that is disposed on the outside of the stent (not shown) differs substantially from the radio-frequency field inside of the uncoated stent (not shown). In some cases, because of substrate effects, substantially none of such radio-frequency signal passes through the uncoated stent (not shown).

In the third step of this process, and in one embodiment thereof, the MRI field(s) interact with material disposed on the inside of coated stent 400 such as, e.g., plaque particles 430 and 432. This interaction produces a signal 441 by means well known to those in the MRI imaging art.

In the fourth step of the preferred process of this invention, the signal 441 passes back through the coated stent 400 in a manner such that it is substantially unaffected by the coated stent 400. Thus, in this embodiment, the radio-frequency field that is disposed on the inside of the coated stent 400 is substantially the same as the radio-frequency field that passes through and is disposed on the outside of the coated stent 400.

By comparison, when the stent (not shown) is not coated with the coatings of this invention, the characteristics of the signal 441 are substantially varied by its passage through the uncoated stent. Thus, with such uncoated stent, the radio-frequency signal that is disposed on the inside of the stent (not shown) differs substantially from the radio-frequency field outside of the uncoated stent (not shown). In some cases, because of substrate effects, substantially none of such signal 441 passes through the uncoated stent (not shown).

Another Preferred Process of the Invention

FIGS. 17A, 17B, and 17C illustrate another preferred process of the invention in which a medical device (such as, e.g., a stent 2200) may be imaged with an MRI imaging process. In the embodiment depicted in FIG. 17A, the stent 2200 is comprised of plaque 2202 disposed inside the inside wall 2204 of the stent 2200.

FIG. 17B illustrates three images produced from the imaging of stent 2200, depending upon the orientation of such stent 2200 in relation to the MRI imaging apparatus reference line (not shown). With a first orientation, an image 2206 is produced. With a second orientation, an image 2208 is produced. With a third orientation, an image 2210 is produced.

By comparison, FIG. 17C illustrates the images obtained when the stent 2200 has the nanomagnetic coating of this invention disposed about it. Thus, when the coated stent 400 of FIG. 9 is imaged, the images 2212, 2214, and 2216 are obtained.

The images 2212, 2214, and 2216 are obtained when the coated stent 400 is at the orientations of the uncoated stent 2200 the produced images 2206, 2208, and 2210, respectively. However, as will be noted, despite the variation in orientations, one obtains the same image with the coated stent 400.

Thus, e.g., the image 2218 of the coated stent (or other coated medical device) will be identical regardless of how such coated stent (or other coated medical device) is oriented vis-a-vis the MRI imaging apparatus reference line (not shown). Thus, e.g., the image 2220 of the plaque particles will be the same regardless of how such coated stent is oriented vis-a-vis the MRI imaging apparatus reference line (not shown).

Consequently, in this embodiment of the invention, one may utilize a nanomagnetic coating that, when imaged with the MRI imaging apparatus, will provide a distinctive and reproducible imaging response regardless of the orientation of the medical device.

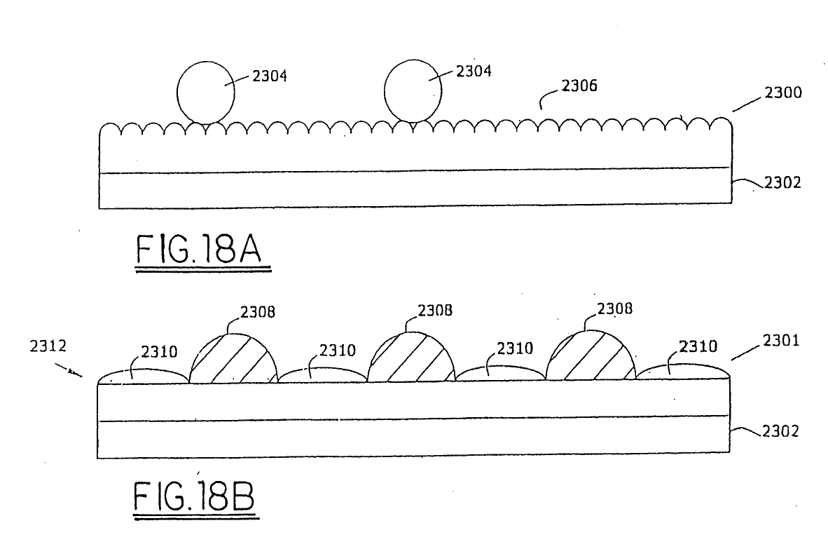

FIGS. 18A and 18B illustrate a hydrophobic coating 2300 and a hydrophilic coating 2301 that may be produced by the process of this invention.

As is known to those skilled in the art, a hydrophobic material is antagonistic to water and incapable of dissolving in water. A hydrophobic surface is illustrated in FIG. 18A.

Referring to FIG. 18A, it will be seen that a coating 2300 is deposited onto substrate 2302. In the embodiment depicted, the coating 2300 an average surface roughness of less than about 1 nanometer. Inasmuch as the average water droplet has a minimum cross-sectional dimension of at least about 3 nanometers, the water droplets 2304 will tend not to bond to the coated surface 2306 which, thus, is hydrophobic with regard to such water droplets.

One may vary the average surface roughness of coated surface 2306 by varying the pressure used in the sputtering process described elsewhere in this specification. In general, the higher the gas pressure used, the rougher the surface.

FIG. 18BB illustrates water droplets 2308 between surface features 2310 of coated surface 2312. In this embodiment, because the surface features 2310 are spaced from each other by a distance of at least about 10 nanometers, the water droplets 2308 have an opportunity to bond to the surface 2312 which, in this embodiment, is hydrophilic.

The Bond Formed Between the Substrate and the Coating

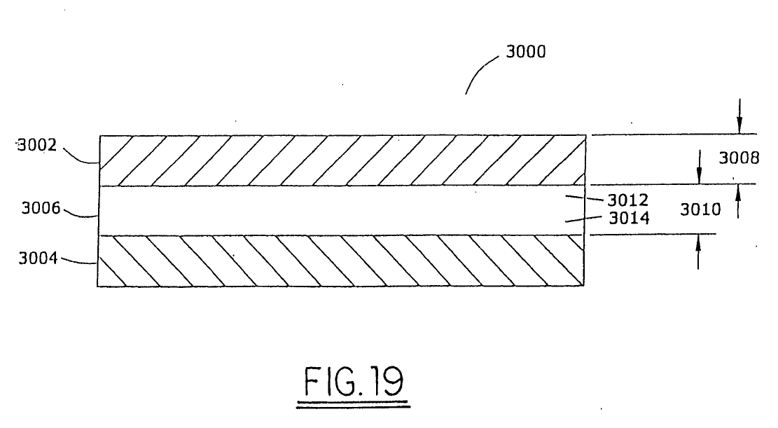

Applicants believe that, in at least one preferred embodiment of the process of their invention, the particles in their coating diffuse into the substrate being coated to form a interfacial diffusion layer. This structure is best illustrated in FIG. 19 which, as will be apparent, is not drawn to scale.

Referring to FIG. 19, the coated assembly 3000 is preferably comprised of a coating 3002 disposed on a substrate 3004. The coating 3002 preferably has at thickness 3008 of at least about 150 nanometers.

The interlayer 3006, by comparison, has a thickness of 3010 of less than about 10 nanometers and, preferably, less than about 5 nanometers. In one embodiment, the thickness of interlayer 3010 is less than about 2 nanometers.

The interlayer 3006 is preferably comprised of a heterogeneous nixture of atoms from the substrate 3004 and the coating 3002. It is preferred that at least 10 mole percent of the atoms from the coating 3002 are present in the interlayer 3006, and that at least 10 mole percent of the atoms from the substrate 3004 are in the interlayer 3006. It is more preferred that from about 40 to about 60 mole percent of the atoms from each of the coating and the substrate be present in the interlayer 3006, it being apparent that more atoms from the coating will be present in that portion 3012 of the interlayer closest to the coating, and more atoms from the substrate will be present in that portion 3014 closest to the substrate.

In one embodiment, the substrate 3004 will consist essentially of niobium atoms with from about 0 to about 2 molar percent of zirconium atoms present. In another embodiment, the substrate 3004 will comprise nickel atoms and titanium atoms . In yet another embodiment, the substrate will comprise tantalum atoms, or titanium atoms.

The coating may comprise any of the A, B, and/or C atoms described hereinabove. By way of way of illustration, the coating may comprise aluminum atoms and oxygen atoms (in the form of aluminum oxide), iridium atoms and oxygen atoms (in the form of irdium oxide), etc.

A Coated Substrate With a Specified Surface Morphology

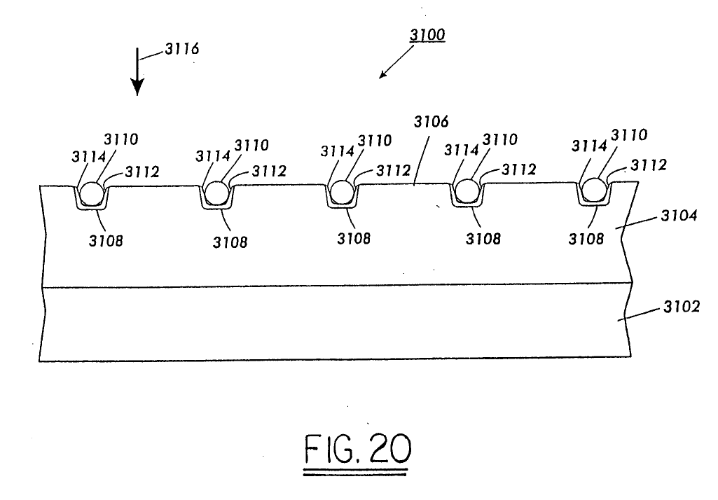

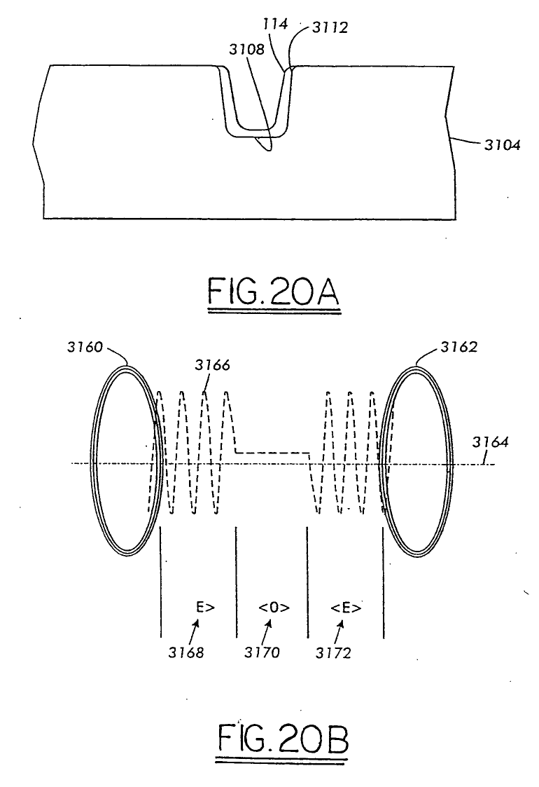

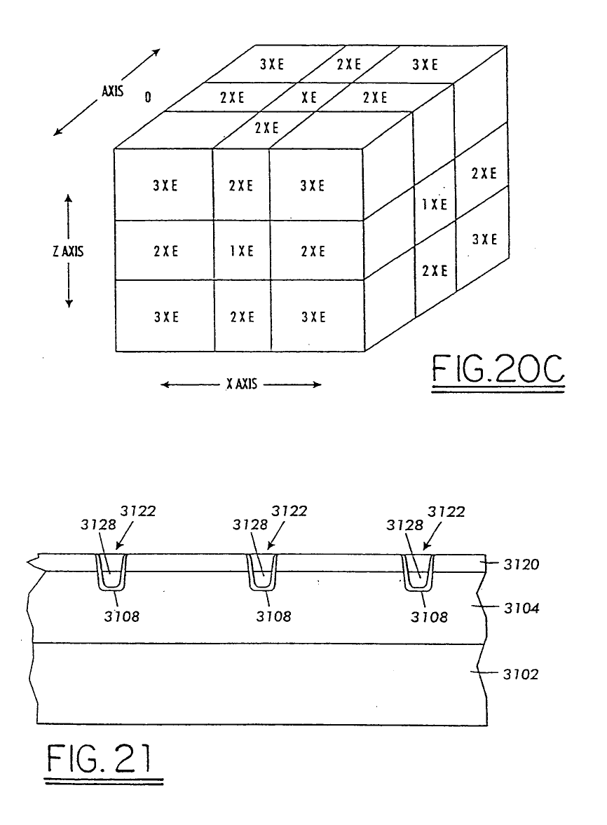

FIG. 20 is a sectional schematic view of a coated substrate 3100 comprised of a substrate 3102 and, bonded thereto, a layer 3104 of nano-sized particles that may comprise nanomagnetic particles, nanoelectrical particles, nanoinsulative particles, nanothermal particles. These particles, the mixtures thereof, and the matrices in which they are disposed have all been described elsewhere in this specification. Depending upon the properties desired from the coated substrate 3100 and/or the layer 3104, one may use one or more of the coating constructs described elsewhere in this specification. Thus, e.g., depending upon the type of particle(s) used and its properties, one may produce a desired set of electrical and magnetic properties for either the coated substrate 3100, the substrate 3200, and/or the coating 3104.

In one embodiment, the coating 3104 is comprised of at least about 5 weight percent of nanomagnetic material with the properties described elsewhere in this specification. In another embodiment, the coating 3104 is comprised of at least 10 weight percent of nanomagnetic material. In yet another embodiment, the coating 3104 is comprised of at least about 40 weight percent of nanomagnetic material.

Referring again to FIG. 20, and to the preferred embodiment depicted therein, the surface 3106 of the coating 3104 is comprised of a multiplicity of morphological indentations 3108 sized to receive drug particles 3110.

In one embodiment, the drug particles are particles of an anti-microtubule agent, as that term is described and defined in U.S. Pat. No. 6,333,347. The entire disclosure of this U.S. patents is hereby incorporated by reference into this specification.

As is known to those skilled in the art, paclitaxel is an anti-microtubule agent. As that term is used in this specification (and as it also is used in the specification of U.S. Pat. No. 6,333,347), the term “anti-microtubule agent” includes any protein, peptide, chemical, or other molecule which impairs the function of microtubules, for example, through the prevention or stabilization of polymerization. As is known to those in the art, a wide variety of methods may be utilized to determine the anti-microtubule activity of a particular compound, including for example, assays described by Smith et al. (Cancer Lett 79(2):213-219, 1994) and Mooberry et al., (Cancer Lett. 96(2):261-266, 1995).

As is disclosed at columns 3-5 of U.S. Pat. No. 6,333,347, “. . . a wide variety of anti-microtubule agents may be delivered, either with or without a carrier (e.g., a polymer or ointment), in order to treat or prevent disease. Representative examples of such agents include taxanes (e.g., paclitaxel (discussed in more detail below) and docetaxel) (Schiff et al., Nature 277: 665-667, 1979; Long and Fairchild, Cancer Research 54: 4355-4361, 1994; Ringel and Horwitz, J. Natl. Cancer Inst. 83(4): 288-291, 1991; Pazdur et al., Cancer Treat. Rev. 19(4): 351-386, 1993), campothecin, eleutherobin (e.g., U.S. Pat. No. 5,473,057), sarcodictyins (including sarcodictyin A), epothilones A and B (Bollag et al., Cancer Research 55: 2325-2333, 1995), discodermolide (ter Haar et al., Biochemistry 35: 243-250, 1996), deuterium oxide (D2O) (James and Lefebvre, Genetics 130(2): 305-314, 1992; Sollott et al., J. Clin. Invest. 95: 1869-1876, 1995), hexylene glycol (2-methyl-2,4-pentanediol) (Oka et al., Cell Struct. Funct. 16(2): 125-134, 1991), tubercidin (7-deazaadenosine) (Mooberry et al., Cancer Lett. 96(2): 261-266, 1995), LY290181 (2-amino-4-(3-pyridyl)-4H-naphtho(1,2-b)pyran-3-cardonitrile) (Panda et al., J. Biol. Chem. 272(12): 7681-7687, 1997; Wood et al., Mol. Pharmacol. 52(3): 437-444, 1997), aluminum fluoride (Song et al., J. Cell. Sci. Suppl. 14: 147-150, 1991), ethylene glycol bis-(succinimidylsuccinate) (Caplow and Shanks, J. Biol. Chem. 265(15): 8935-8941, 1990), glycine ethyl ester (Mejillano et al., Biochemistry 31(13): 3478-3483, 1992), nocodazole (Ding et al., J. Exp. Med. 171(3): 715-727, 1990; Dotti et al., J. Cell Sci. Suppl. 15: 75-84, 1991; Oka et al., Cell Struct. Funct. 16(2): 125-134, 1991; Weimer et al., J. Cell. Biol. 136(1), 71-80, 1997), cytochalasin B (Illinger et al., Biol. Cell 73(2-3): 131-138, 1991), colchicine and CI 980 (Allen et al., Am. J. Physiol. 261(4 Pt. 1) L315-L321, 1991; Ding et al., J. Exp. Med. 171(3): 715-727, 1990; Gonzalez et al., Exp. Cell. Res. 192(1): 10-15, 1991; Stargell et al., Mol. Cell. Biol. 12(4): 1443-1450, 1992; Garcia et al., Antican. Drugs 6(4): 533-544, 1995), colcemid (Barlow et al., Cell. Motil. Cytoskeleton 19(1): 9-17, 1991; Meschini et al., .J Microsc. 176(Pt. 3): 204-210, 1994; Oka et al., Cell Struct. Funct. 16(2): 125-134, 1991), podophyllotoxin (Ding et al., J. Exp. Med 171(3): 715-727, 1990), benomyl (Hardwick et al., J. Cell. Biol. 131(3): 709-720, 1995; Shero et al., Genes Dev. 5(4): 549-560, 1991), oryzalin (Stargell et al., Mol. Cell. Biol. 12(4): 1443-1450, 1992), majuscularnide C (Moore, J. Ind. Microbiol. 16(2): 134-143, 1996), demecolcine (Van Dolah and Ramsdell, J. Cell. Physiol. 166(1): 49-56, 1996; Wiemer et al., J. Cell. Biol. 136(1): 71-80, 1997), methyl-2-benzimidazolecarbamate (MBC) (Brown et al., J. Cell. Biol. 123(2): 387-403, 1993), LY195448 (Barlow & Cabral, Cell Motil. Cytoskel. 19: 9-17, 1991), subtilisin (Saoudi et al., J. Cell Sci. 108: 357-367, 1995), 1069C85 (Raynaud et al., Cancer Chemother. Pharmacol. 35: 169-173, 1994), steganacin (Hamel, Med Res. Rev. 16(2): 207-231, 1996), combretastatins (Hamel, Med Res. Rev. 16(2): 207-231, 1996), curacins (Hamel, Med Res. Rev. 16(2): 207-231, 1996), estradiol (Aizu-Yokata et al., Carcinogen.. 15(9): 1875-1879, 1994), 2-methoxyestradiol (Hamel, Med Res. Rev. 16(2): 207-231, 1996), flavanols (Hamel, Med Res. Rev. 16(2): 207-231, 1996), rotenone (Hamel, Med Res. Rev. 16(2): 207-231, 1996), griseofulvin (Hamel, Med Res. Rev. 16(2): 207-231, 1996), vinca alkaloids, including vinblastine and vincristine (Ding et al., J. Exp. Med 171(3): 715-727, 1990; Dirk et al., Neurochem. Res. 15(11): 1135-1139, 1990; Hamel, Med Res. Rev. 16(2): 207-231, 1996; Illinger et al., Biol. Cell 73(2-3): 131-138, 1991; Wiemer et al., J. Cell. Biol. 136(1): 71-80, 1997), maytansinoids and ansanitocins (Hamel, Med Res. Rev. 16(2): 207-231, 1996), rhizoxin (Hamel, Med Res. Rev. 16(2): 207-231, 1996), phomopsin A (Hamel, Med. Res. Rev. 16(2): 207-231, 1996), ustiloxins (Hamel, Med Res. Rev. 16(2): 207-231, 1996), dolastatin 10 (Hamel, Med. Res. Rev. 16(2): 207-231, 1996), dolastatin 15 (Hamel, Med. Res. Rev. 16(2): 207-231, 1996), halichondrins and halistatins (Hamel, Med. Res. Rev. 16(2): 207-231, 1996), spongistatins (Hamel, Med Res. Rev. 16(2): 207-231, 1996), cryptophycins (Hamel, Med. Res. Rev. 16(2): 207-231, 1996), rhazinilam (Hamel, Med. Res. Rev. 16(2): 207-231, 1996), betaine (Hashimoto et al., Zool. Sci. 1: 195-204, 1984), taurine (Hashimoto et al., Zool. Sci. 1: 195-204, 1984), isethionate (Hashimoto et al., Zool. Sci. 1: 195-204, 1984), HO-221 (Ando et al., Cancer Chemother. Pharmacol. 37: 63-69, 1995), adociasulfate-2 (Sakowicz et al., Science 280: 292-295, 1998), estramustine (Panda et al., Proc. Natl. Acad. Sci. USA 94: 10560-10564, 1997), monoclonal anti-idiotypic antibodies (Leu et al., Proc. Natl. Acad. Sci. USA 91(22): 10690-10694, 1994), microtubule assembly promoting protein (paclitaxel-like protein, TALP) (Hwang et al., Biochem. Biophys. Res. Commun. 208(3): 1174-1180, 1995), cell swelling induced by hypotonic (190 mosmol/L) conditions, insulin (100 nmol/L) or glutamine (10 mmol/L) (Haussinger et al., Biochem. Cell. Biol. 72(1-2): 12-19, 1994), dynein binding (Ohba et al., Biochim. Biophys. Acta 1158(3): 323-332, 1993), gibberelin (Mita and Shibaoka, Protoplasma 119(1/2): 100-109, 1984), XCHO1 (kinesin-like protein) (Yonetani et al., Mol. Biol. Cell 7(suppl): 211A, 1996), lysophosphatidic acid (Cook et al., Mol. Biol Cell 6(suppl): 260A, 1995), lithium ion (Bhattacharyya and Wolff, Biochem. Biophys. Res. Commun. 73(2): 383-390, 1976), plant cell wall components (e.g., poly-L-lysine and extensin) (Akashi et al., Planta 182(3): 363-369, 1990), glycerol buffers (Schilstra et al., Biochem. J. 277(Pt. 3): 839-847, 1991; Farrell and Keates, Biochem. Cell. Biol. 68(11): 1256-1261, 1990; Lopez et al., J. Cell. Biochem. 43(3): 281-291, 1990), Triton X-100 microtubule stabilizing buffer (Brown et al., J. Cell Sci. 104(Pt. 2): 339-352, 1993; Safiejko-Mroczka and Bell, J. Histochem. Cytochem. 44(6): 641-656, 1996), microtubule associated proteins (e.g, MAP2, MAP4, tau, big tau, ensconsin, elongation factor-1-alpha (EF-1.alpha.) and E-MAP-115) (Burgess et al., Cell Motil. Cytoskeleton 20(4): 289-300, 1991; Saoudi et al., J. Cell. Sci. 108(Pt. 1): 357-367, 1995; Bulinski and Bossler, J. Cell. Sci. 107(Pt. 10): 2839-2849, 1994; Ookata et al., J. Cell Biol. 128(5): 849-862, 1995; Boyne et al., J. Comp. Neurol. 358(2): 279-293, 1995; Ferreira and Caceres, J. Neurosci. 11(2): 392-400, 1991; Thurston et al., Chromosoma 105(1): 20-30, 1996; Wang et al., Brain Res. Mol. Brain Res. 38(2): 200-208, 1996; Moore and Cyr, Mol. Biol. Cell 7(suppl): 221-A, 1996; Masson and Kreis, J. Cell Biol. 123(2), 357-371, 1993), cellular entities (e.g., histone HI, myelin basic protein and kinetochores) (Saoudi et al., J. Cell. Sci. 108(Pt. 1): 357-367, 1995; Simerly et al., J. Cell Biol. 111(4): 1491-1504, 1990), endogenous microtubular structures (e.g., axonemal structures, plugs and GTP caps) (Dye et al., Cell Motil. Cytoskeleton 21(3): 171-186, 1992; Azhar and Murphy, Cell Motil. Cytoskeleton 15(3): 156-161, 1990; Walkeret al., J. Cell Biol. 114(1): 73-81, 1991; Drechsel and Kirschner, Curr. Biol. 4(12): 1053-1061, 1994), stable tubule only polypeptide (e.g., STOP145 and STOP220) (Pirollet et al., Biochim. Biophys. Acta 1160(1): 113-119, 1992; Pirollet et al., Biochemistry 31(37): 8849-8855, 1992; Bosc et al., Proc. Natl. Acad. Sci. USA 93(5): 2125-2130, 1996; Margolis et al., EMBO J. 9(12): 4095-4102, 1990) and tension from mitotic forces (Nicklas and Ward, J. Cell Biol. 126(5): 1241-1253, 1994), as well as any analogues and derivatives of any of the above. Such compounds can act by either depolymerizing microtubules (e.g., colchicine and vinblastine), or by stabilizing microtubule formation (e.g., paclitaxel).”

One preferred anti-microtuble agent is paclitaxel, a compound which disrupts microtubule formation by binding to tubulin to form abnormal mitotic spindles. As is disclosed at columns 5-6 of such U.S. Pat. No. 6,333,347 (the entire disclosure of which is hereby incorporated by reference into this specification), “. . . paclitaxel is a highly derivatized diterpenoid (Wani et al., J. Am. Chem. Soc. 93:2325, 1971) which has been obtained from the harvested and dried bark of Taxus brevifolia (Pacific Yew) and Taxomyces Andreanae and Endophytic Fungus of the Pacific Yew (Stierle et al., Science 60:214-216, 1993). ‘Paclitaxel’ (which should be understood herein to include prodrugs, analogues and derivatives such as, for example, PACLITAXEL®, TAXOTERE®, Docetaxel, 10-desacetyl analogues of paclitaxel and 3′N-desbenzoyl-3′N-t-butoxy carbonyl analogues of paclitaxel) may be readily prepared utilizing techniques known to those skilled in the art (see e.g., Schiff et al., Nature 277:665-667, 1979; Long and Fairchild, Cancer Research 54:4355-4361, 1994; Ringel and Horwitz, J. Natl. Cancer Inst. 83(4):288-291, 1991; Pazdur et al., Cancer Treat. Rev. 19(4):351-386, 1993; WO 94/07882; WO 94/07881; WO 94/07880; WO 94/07876; WO 93/23555; WO 93/10076; WO 94/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52):9709-9712, 1994; J. Med Chem. 35:4230-4237, 1992; J. Med. Chem. 34:992-998, 1991; J. Natural Prod. 57(10):1404-1410, 1994; J. Natural Prod. 57(11):1580-1583, 1994; J. Am. Chem. Soc. 110:6558-6560, 1988), or obtained from a variety of commercial sources, including for example, Sigma Chemical Co., St. Louis, Mo. (T7402—from Taxus brevifolia).” The entire disclosure of each of the U.S. patents described in this paragraph of the specification is hereby incorporated by reference into this specification.

Paclitaxel derivatives and/or analogues are also drugs which may be used in the process of this invention. As is disclosed at columns 5-6 of such U.S. Pat. No. 6,333,347, “Representative examples of such paclitaxel derivatives or analogues include 7-deoxy-docepaclitaxel, 7,8-cyclopropataxanes, N-substituted 2-azetidones, 6,7-epoxy paclitaxels, 6,7-modified paclitaxels, 10-desacetoxypaclitaxel, 10-deacetylpaclitaxel (from 10-deaceiylbaccatin III), phosphonooxy and carbonate derivatives of paclitaxel, paclitaxel 2′,7-di(sodium 1,2-benzenedicarboxylate, 10-desacetoxy-11,12-dihydropaclitaxel-10,12(18)-diene derivatives, 10-desacetoxypaclitaxel, Propaclitaxel (2′- and/or 7-O-ester derivatives ), (2′-and/or 7-O-carbonate derivatives), asymmetric synthesis of paclitaxel side chain, fluoro paclitaxels, 9-deoxotaxane, (13-acetyl-9-deoxobaccatine III, 9-deoxopaclitaxel, 7-deoxy-9-deoxopaclitaxel, 10-desacetoxy-7-deoxy-9-deoxopaclitaxel, Derivatives containing hydrogen or acetyl group and a hydroxy and tert-butoxycarbonylamino, sulfonated 2′-acryloylpaclitaxel and sulfonated 2′-O-acyl acid paclitaxel derivatives, succinylpaclitaxel, 2′-.gamma.-aminobutyrylpaclitaxel formate, 2′-acetyl paclitaxel, 7-acetyl paclitaxel, 7-glycine carbamate paclitaxel, 2′-OH-7-PEG(5000) carbamate paclitaxel, 2′-benzoyl and 2′,7-dibenzoyl paclitaxel derivatives, other prodrugs (2′-acetylpaclitaxel; 2′,7-diacetylpaclitaxel; 2′succinylpaclitaxel; 2′-(beta-alanyl)-paclitaxel); 2′gamma-amninobutyrylpaclitaxel formate; ethylene glycol derivatives of 2′-succinylpaclitaxel; 2′-glutarylpaclitaxel; 2′-(N,N-dimethylglycyl) paclitaxel; 2′-(2-(N,N-dimethylamino)propionyl)paclitaxel; 2′orthocarboxybenzoyl paclitaxel; 2′aliphatic carboxylic acid derivatives of paclitaxel, Prodrugs {2′(N,N-diethylaminopropionyl)paclitaxel, 2′(N,N-dimethylglycyl)paclitaxel, 7(N,N-dimethylglycyl)paclitaxel, 2′,7-di-(N,N-dimethylglycyl)paclitaxel, 7(N,N-diethylaminopropionyl)paclitaxel, 2′,7-di(N,N-diethylaminopropionyl)paclitaxel, 2′-(L-glycyl)paclitaxel, 7-(L-glycyl)paclitaxel, 2′,7-di(L-glycyl)paclitaxel, 2′-(L-alanyl)paclitaxel, 7-(L-alanyl)paclitaxel, 2′,7-di(L-alanyl)paclitaxel, 2′-(L-leucyl)paclitaxel, 7-(L-leucyl)paclitaxel, 2′,7-di(L-leucyl)paclitaxel, 2′-(L-isoleucyl)paclitaxel, 7-(L-isoleucyl)paclitaxel, 2′,7-di(L-isoleucyl)paclitaxel, 2′-(L-valyl)paclitaxel, 7-(L-valyl)paclitaxel, 2′7-di(L-valyl)paclitaxel, 2′-(L-phenylalanyl)paclitaxel, 7-(L-phenylalanyl)paclitaxel, 2′,7-di(L-phenylalanyl)paclitaxel, 2′-(L-prolyl)paclitaxel, 7-(L-prolyl)paclitaxel, 2′,7-di(L-prolyl)paclitaxel, 2′-(L-lysyl)paclitaxel, 7-(L-lysyl)paclitaxel, 2′,7-di(L-lysyl)paclitaxel, 2′-(L-glutamyl)paclitaxel, 7-(L-glutamyl)paclitaxel, 2′,7-di(L-glutamyl)paclitaxel, 2′-(L-arginyl)paclitaxel, 7-(L-arginyl)paclitaxel, 2′,7-di(L-arginyl)paclitaxel }, Paclitaxel analogs with modified phenylisoserine side chains, taxotere, (N-debenzoyl-N-tert-(butoxycaronyl)-10-deacetylpaclitaxel, and taxanes (e.g., baccatin III, cephalomannine, 10-deacetylbaccatin III, brevifoliol, yunantaxusin and taxusin).”

In the process of this invention, the anti-microtubule agent may be utilized by itself, and/or it may be utilized in a formulation that comprises such agent and a carrier. The carrier may be either of polymeric or non-polymeric origin; it may, e.g., be one or more of the polymeric materials 14 (see FIGS. 1 and 1A) described elsewhere in this specification.. Many suitable carriers for anti-microtubule agents are disclosed at columns 6-9 of such U.S. Pat. No. 6,333,347.

Thus, e.g., and as is disclosed in U.S. Pat. No. 6,333,347, “. . . a wide variety of polymeric carriers may be utilized to contain and/or deliver one or more of the therapeutic agents discussed above, including for example both biodegradable and non-biodegradable compositions. Representative examples of biodegradable compositions include albumin, collagen, gelatin, hyaluronic acid, starch, cellulose (methylcellulose, hydroxypropylcellulose, hydroxypropylmethylcellulose,, hydroxyethylcellulose, carboxymethylcellulose, cellulose acetate phthalate, cellulose acetate succinate, hydroxypropylmethylcellulose phthalate), casein, dextrans, polysaccharides, fibrinogen, poly(D,L lactide), poly(D,L-lactide-coglycolide), poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids) and their copolymers (see generally, ilium, L., Davids, S. S. (eds.) “Polymers in Controlled Drug Delivery” Wright, Bristol, 1987; Arshady, J. Controlled Release 17:1-22, 1991; Pitt, Int. J. Phar. 59:173-196, 1990; Holland et al., J. Controlled Release 4:155-0180, 1986). Representative examples of nondegradable polymers include poly(ethylene-vinyl acetate) (“EVA”) copolymers, silicone rubber, acrylic polymers (polyacrylic acid, polymethylacrylic acid, polymethylmethacrylate, polyalkylcynoacrylate), polyethylene, polyproplene, polyamides (nylon 6,6), polyurethane, poly(ester urethanes), poly(ether urethanes), poly(ester-urea), polyethers (poly(ethylene oxide), poly(propylene oxide), Pluronics and poly(tetramethylene glycol)), silicone rubbers and vinyl polymers (polyvinylpyrrolidone, poly(vinyl alcohol), poly(vinyl acetate phthalate). Polymers may also be developed which are either anionic (e.g., alginate, carrageenin, carboxymethyl cellulose and poly(acrylic acid), or cationic (e.g, chitosan, poly-L-lysine, polyethylenimine, and poly (allyl amine)) (see generally, Dunn et al., J. Applied Polymer Sci. 50:353-365, 1993; Cascone et al., J. Materials Sci. Materials in Medicine 5:770-774, 1994; Shiraishi et al., Biol. Pharm. Bull. 16(11):1164-1168, 1993; Thacharodi and Rao, Int'l J. Pharm. 120:115-118, 1995; Miyazaki et al., Int'l J. Pharm. 118:257-263, 1995). Particularly preferred polymeric carriers include poly(ethylene-vinyl acetate), poly (D,L-lactic acid) oligomers and polymers, poly (L-lactic acid) oligomers and polymers, poly (glycolic acid), copolymers of lactic acid and glycolic acid, poly (caprolactone), poly (valerolactone), polyanhydrides, copolymers of poly (caprolactone) or poly (lactic acid) with a polyethylene glycol (e.g., MePEG), and blends thereof.” These polymeric carrier materials also may be utilized as the polymeric material 14 (see FIGS. 1 and 1A).

As is also disclosed in U.S. Pat. No. 6,333,347, “Polymeric carriers can be fashioned in a variety of forms, with desired release characteristics and/or with specific desired properties. For example, polymeric carriers may be fashioned to release a therapeutic agent upon exposure to a specific triggering event such as pH (see e.g., Heller et al., “Chemically Self-Regulated Drug Delivery Systems,” in Polymers in Medicine Ell, Elsevier Science Publishers B. V., Amsterdam, 1988, pp. 175-188; Kang et al., J. Applied Polymer Sci. 48:343-354, 1993; Dong et al., J. Controlled Release 19.171-178, 1992; Dong and Hoffman, J. Controlled Release 15:141-152, 1991; Kim et al., J. Controlled Release 28:143-152, 1994; Cornejo-Bravo et al., J. Controlled Release 33:223-229, 1995; Wu and Lee, Pharm. Res. 10(10):1544-1547, 1993; Serres et al., Pharm. Res. 13(2):196-201, 1996; Peppas, “Fundamentals of pH- and Temperature-Sensitive Delivery Systems,” in Gurny et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers I, Springer-Verlag, Berlin). Representative examples of pH-sensitive polymers include poly(acrylic acid) and its derivatives (including for example, homopolymers such as poly(aminocarboxylic acid); poly(acrylic acid); poly(methyl acrylic acid), copolymers of such homopolymers, and copolymers of poly(acrylic acid) and acrylmonomers such as those discussed above. Other pH sensitive polymers include polysaccharides such as cellulose acetate phthalate; hydroxypropylmethylcellulose phthalate; hydroxypropylmethylcellulose acetate succinate; cellulose acetate trimellilate; and chitosan. Yet other pH sensitive polymers include any mixture of a pH sensitive polymer and a water soluble polymer.”

As is also disclosed in U.S. Pat. No. 6,333,347, “Likewise, polymeric carriers can be fashioned which are temperature sensitive (see e.g., Chen et al., “Novel Hydrogels of a Temperature-Sensitive Pluronic Grafted to a Bioadhesive Polyacrylic Acid Backbone for Vaginal Drug Delivery,” in Proceed Intern. Symp. Control. Rel. Bioact. Mater. 22:167-168, Controlled Release Society, Inc., 1995; Okano, “Molecular Design of Stimuli-Responsive Hydrogels for Temporal Controlled Drug Delivery,” in Proceed Intern. Symp. Control. Rel. Bioact. Mater. 22:111-112, Controlled Release Society, Inc., 1995; Johnston et al., Pharm. Res. 9(3):425-433, 1992; Tung, Int'l J. Pharm. 107:85-90, 1994; Harsh and Gehrke, J. Controlled Release 17:175-186, 1991; Bae et al., Pharm. Res. 8(4):531-537, 1991; Dinarvand and D'Emanuele, J.. Controlled Release 36:221-227, 1995; Yu and Grainger, “Novel Thermo-sensitive Amphiphilic Gels: Poly N-isopropylacrylamide-co-sodium acrylate-co-n-N-alkylacrylamide Network Synthesis and Physicochemical Characterization,” Dept. of Chemical & Biological Sci., Oregon Graduate Institute of Science & Technology, Beaverton, Oreg., pp. 820-821; Zhou and Smid, “Physical Hydrogels of Associative Star Polymers,” Polymer Research Institute, Dept. of Chemistry, College of Environmental Science and Forestry, State Univ. of New York, Syracuse, N.Y., pp. 822-823; Hoffman et al., “Characterizing Pore Sizes and Water ‘Structure’ in Stimuli-Responsive Hydrogels,” Center for Bioengineering, Univ. of Washington, Seattle, Wash., p. 828; Yu and Grainger, “Thermo-sensitive Swelling Behavior in Crosslinked N-isopropylacrylamide Networks: Cationic, Anionic and Ampholytic Hydrogels,” Dept. of Chemical & Biological Sci., Oregon Graduate Institute of Science & Technology, Beaverton, Oreg., pp. 829-830; Kim et al., Pharm. Res. 9(3):283-290, 1992; Bae et al., Pharm. Res. 8(5):624-628, 1991; Kono et al., J. Controlled Release 30:69-75, 1994; Yoshida et al., J. Controlled Release 32:97-102, 1994; Okano et al., J. Controlled Release 36:125-133, 1995; Chun and Kim, J. Controlled Release 38:39-47, 1996; D'Emanuele and Dinarvand, Int'l J. Pharm. 118:237-242, 1995; Katono et al., J. Controlled Release 16:215-228, 1991; Hoffman, “Thermally Reversible Hydrogels Containing Biologically Active Species,” in Migliaresi et al. (eds.), Polymers in Medicine III, Elsevier Science Publishers B. V., Amsterdam, 1988, pp. 161-167; Hoffman, “Applications of Thermally Reversible Polymers and Hydrogels in Therapeutics and Diagnostics,” in Third International Symposium on Recent Advances in Drug Delivery Systems, Salt Lake City, Utah, Feb. 24-27, 1987, pp. 297-305; Gutowska et al., J. Controlled Release 22:95-104, 1992; Palasis and Gehrke, J. Controlled Release 18:1-12, 1992; Paavolaetal., Pharm. Res. 12(12):1997-2002, 1995).”

As is also disclosed in U.S. Pat. No. 6,333,347, “Representative examples of thermogelling polymers, and their gelatin temperature (LCST (° C.)) include homopolymers such as poly(N-methyl-N-n-propylacrylamide), 19.8; poly(N-n-propylacrylamide), 21.5; poly(N-methyl-N-isopropylacrylamide), 22.3; poly(N-n-propylmethacrylamide), 28.0; poly(N-isopropylacrylamide), 30.9; poly(N, n-diethylacrylamide), 32.0; poly(N-isopropylmethacrylarnide), 44.0; poly(N-cyclopropylacrylamide), 45.5; poly(N-ethylmethyacrylamide), 50.0; poly(N-methyl-N-ethylacrylamide), 56.0; poly(N-cyclopropylmethacrylamide), 59.0; poly(N-ethylacrylamide), 72.0. Moreover thermogelling polymers may be made by preparing copolymers between (among) monomers of the above, or by combining such homopolymers with other water soluble polymers such as acrylmonomers (e.g. acrylic acid and derivatives thereof such as methylacrylic acid, acrylate and derivatives thereof such as butyl methacrylate, acrylamide, and N-n-butyl acrylamide).”

As is also disclosed in U.S. Pat. No. 6,333,347, “Other representative examples of thermogelling polymers include cellulose ether derivatives such as hydroxypropyl cellulose, 41° C.; methyl cellulose, 55° C.; hydroxypropylmethyl cellulose, 66° C.; and ethylhydroxyethyl cellulose, and Pluronics such as F-127, 10-15° C.; L-122, 19° C.; L-92, 26° C.; L-81, 20° C.; and L-61, 24° C.”

As is also disclosed in U.S. Pat. No. 6,333,347, “A wide variety of forms may be fashioned by the polymeric carriers of the present invention, including for example, rod-shaped devices, pellets, slabs, or capsules (see e.g., Goodell et al., Am. J. Hosp. Pharm. 43:1454-1461, 1986; Langer et al., ‘Controlled release of macromolecules from polymers’, in Biomedical Polymers, Polymeric Materials and Pharmaceuticals for Biomedical Use, Goldberg, E. P., Nakagim, A. (eds.) Academic Press, pp. 113-137, 1980; Rhine et al., J. Pharm. Sci. 69:265-270, 1980; Brown et al., J. Pharm. Sci. 72:1181-1185, 1983; and Bawa et al., J. Controlled Release 1:259-267, 1985). Therapeutic agents may be linked by occlusion in the matrices of the polymer, bound by covalent linkages, or encapsulated in microcapsules. Within certain preferred embodiments of the invention, therapeutic compositions are provided in non-capsular formulations such as microspheres (ranging from nanometers to micrometers in size), pastes, threads of various size, films and sprays.”

As is also disclosed in U.S. Pat. No. 6,333,347, “Preferably, therapeutic compositions of the present invention are fashioned in a manner appropriate to the intended use. Within certain aspects of the present invention, the therapeutic composition should be biocompatible, and release one or more therapeutic agents over a period of several days to months. For example, “quick release” or “burst” therapeutic compositions are provided that release greater than 10%, 20%, or 25% (w/v) of a therapeutic agent (e.g., paclitaxel) over a period of 7 to 10 days. Such “quick release” compositions should, within certain embodiments, be capable of releasing chemotherapeutic levels (where applicable) of a desired agent. Within other embodiments, “low release” therapeutic compositions are provided that release less than 1% (w/v) of a therapeutic agent over a period of 7 to 10 days. Further, therapeutic compositions of the present invention should preferably be stable for several months and capable of being produced and maintained under sterile conditions.”

As is also disclosed in U.S. Pat. No. 6,333,347, “Within certain aspects of the present invention, therapeutic compositions may be fashioned in any size ranging from 50 nm to 500 μm, depending upon the particular use. Alternatively, such compositions may also be readily applied as a “spray”, which solidifies into a film or coating. Such sprays may be prepared from microspheres of a wide array of sizes, including for example, from 0.1 μm to 3 μm, from 10 μm to 30 μm, and from 30 μm to 100 μm.”

As is also disclosed in U.S. Pat. No. 6,333,347, “Therapeutic compositions of the present invention may also be prepared in a variety of “paste” or gel forms. For example, within one embodiment of the invention, therapeutic compositions are provided which are liquid at one temperature (e.g., temperature greater than 37° C., such as 40° C., 45° C., 50° C., 55° C. or 60° C.), and solid or semi-solid at another temperature (e.g., ambient body temperature, or any temperature lower than 37° C.). Such “thermopastes” may be readily made given the disclosure provided herein.” The nanomagnetic particles of this invention may be disposed in a medium so that they are either in a liquid form, a semi-solid form, or a solid form.

The anti-microtuble agents used in one embodiment of the process of this invention may be formulated in a variety of forms suitable for administration; and they may be formulated to contain more than one anti-microtubule agents, to contain a variety of additional compounds, to have certain physical properties such as, e.g., elasticity, a particular melting point, or a specified release rate.

As is disclosed at columns 6-9 of U.S. Pat. No. 6,333,347, the anti-microtubule agents “. . . may be administered either alone, or in combination with pharmaceutically or physiologically acceptable carrier, excipients or diluents. Generally, such carriers should be nontoxic to recipients at the dosages and concentrations employed. Ordinarily, the preparation of such compositions entails combining the therapeutic agent with buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with nonspecific serum albumin are exemplary appropriate diluents.”

As is also disclosed in U.S. Pat. No. 6,333,347, “The anti-microtubule agent can be administered in a dosage which achieves a statistically significant result. In one embodiment, an antimicrotubule agent such as paclitaxel is administered at a dosage ranging from 100 ug to 50 mg, depending on the mode of administration and the type of carrier, if any for delivery. For treatment of restenosis, a single treatment may be provided before, during or after balloon angioplasty or stenting. For the treatment of instent restenosis, the anti-microtubule agent may be administered directly to prevent closure of the stented vessel. For the treatment of atherosclerosis, an anti-microtubule agent such as paclitaxel may be administered periodically, e.g., once every few months. In the case of cardiac transplantation, the anti-microtubule agent may be delivered in a slow release form that delivers from 1 to 75 mg/m2 (preferably 10 to 50 mg/m2) over a selected period of time. With any of these embodiments, the anti-microtubule agent (e.g., paclitaxel) may be administered along with other therapeutics.”

As is also disclosed in U.S. Pat. No. 6,333,347, “Pericardial administration may be accomplished by a variety of manners including, for example, direct injection (preferably with ultrasound, CT, fluoroscopic, MRI or endoscopic guidance). (See e.g., U.S. Pat. Nos. 5,840,059 and 5,797,870). Within certain embodiments, a Saphenous Vein Harvester such as GSI's ENDOsaph, or Comedicus Inc,.’ PerDUCER (Pericardial Access Device) may be utilized to administer the desired anti-microtubule agent (e.g., paclitaxel).” In one embodiment, an anti-microtubule agent is bonded to the nanomagnetic particles of this invention, and the construct thus made is administered to a patient in one or more of the manners described above.

As is also disclosed in U.S. Pat. No. 6,333,347, “Within one embodiment, the antimicrotubule agent or composition (e.g., paclitaxel and a polymer) may be delivered trans-myocardially through the right or left ventricle.”

As is also disclosed in U.S. Pat. No. 6,333,347, “Within other embodiments, the antimicrotubule agent or composition (e.g., paclitaxel and a polymer) may be administered trans-myocardially through the right atrium. (See, e.g., U.S. Pat. Nos. 5,797,870 and 5,269,326). Briefly, the right atrium lies between the pericardium and the epicardium. An appropriate catheter is guided into the right atrium and positioned parallel with the wall of the pericardium. This positioning allows piercing of the right atrium (either by the catheter, or by an instrument that is passed within the catheter), without risk of damage to either the pericardium or the epicardium. The catheter can then be passed into the pericardial space, or an instrument passed through the lumen of the catheter into the pericardial space.”

As is also disclosed in U.S. Pat. No. 6,333,347, “Alternatively, access to the pericardium, heart, or coronary vasculature may be gained operatively, by, for example, sub-xiphoid entry, a thoracotomy, or, open heart surgery. Preferably, the thoracotomy should be minimal, through an intercostal space for example. Fluoroscopy, or ultrasonic visualization may be utilized to assist in any of these procedures.”

Anti-microtubule Agents With a Magnetic Moment

In one embodiment of the process of this invention, the drug particles 3110 used (see FIG. 20) are particles of an anti-microtubule agent with a magnetic moment.

Illustrative “magnetic moment anti-microtubule agents” are disclosed in applicants' copending U.S. Pat. No. application U.S. Ser. No. 60/516,134, filed on Oct. 31, 2003, the entire disclosure of which is hereby incorporated by reference into this specification.

By way of further illustration, means for producing a composition comprised of magnetic carrier particles having therapeutic quantities of absorbed paclitaxel are known to those skilled in the art. Thus, by way of illustration and not limitation, U.S. Pat. No. 6,200,547 describes: “magnetically controllable, or guided, carrier composition and methods of use and production are disclosed, the composition for carrying biologically active substances to a treatment zone in a body under control of a magnetic field. The composition comprises composite, volume-compounded paclitaxel-adsorbed particles of 0.2 to 5.0 μm in size, and preferably between 0.5 and 5.0 μm, containing 1.0 to 95.0% by mass of carbon, and preferably from about 20% to about 60%. The particles are produced by mechanical milling of a mixture of iron and carbon powders. The obtained particles are placed in a solution of a biologically active substance to adsorb the substance onto the particles. The composition is generally administered in suspension. Magnetic carrier particles having therapeutic quantities of adsorbed paclitaxel, doxorubicin, Tc99, and antisense-C Myc oligonucleotide, an hematoporphyrin derivative, 6-mercaptopurine, Amphotericin B, and Camptothecin have been produced using this invention . . . ”. The entire disclosure of this U.S. patent is hereby incorporated by reference into this specification.

In one embodiment, paclitaxel is bonded to the nanomagnetic particles of this invention in the manner described in U.S. Pat. No. 6,200,547.

By way of yet further illustration, one may use the process of U.S. Pat. No. 6,483,536. This patent describes: “A magnetically controllable, or guided, carrier composition and methods of use and production are disclosed, the composition for carrying biologically active substances to a treatment zone in a body under control of a magnetic field. The composition comprises composite, volume-compounded paclitaxel-adsorbed particles of 0.2 to 5.0 μm in size, and preferably between 0.5 and 5.0 μm, containing 1.0 to 95.0% by mass of carbon, and preferably from about 20% to about 60%. The particles are produced by mechanical milling of a mixture of iron and carbon powders. The obtained particles are placed in a solution of a biologically active substance to adsorb the substance onto the particles. The composition is generally administered in suspension. Magnetic carrier particles having therapeutic quantities of adsorbed paclitaxel, doxorubicin, Tc99, and antisense-C Myc oligonucleotide, an hematoporphyrin derivative, 6-mercaptopurine, Amphotericin B, and Camptothecin have been produced using this invention. Magnetic carrier particles having diagnostic quantities of adsorbed Re186 and Re188 have also been produced using this invention.” The entire disclosure of this U.S. patent is hereby incorporated by reference into this specification. As will be apparent, the process of this patent may be used to adsorb paclitaxel onto the nanomagentic particles of this invention.

By way of yet further illustration, one may enhance the an anti-microtubule agent by using magnetotactic bacteria as a drug carrier that can be directed to the desired site of drug action by guiding the bacteria through the body of a patient via an applied magnetic field whose intensity increases in the vicinity of the desired site.

The preparation and use of magnetotactic bacteria assemblies is well known to those skilled in the art. Thus, and by way of illustration, in U.S. Pat. No. 4,394,451 of Blakemore (the entire disclosure of which is hereby incorporated by reference into this specification), there is described and claimed: “An aqueous culture medium for the growth of a biologically pure culture of magnetic bacteria, comprising, per 100 ml, about 2-30 μM of ferric quinate, about 10-1000 mg of an organic compound selected from the group consisting of fumaric acid, tartaric acid, malic acid, succinic acid, lactic acid, pyruvic acid, oxaloacetic acid, malonic acid, 6-hydroxybutyric acid, maleic acid, galactose, rhamnose, melibiose, acetic acid, adipic acid, and glutaric acid, a vitamin source, a mineral source, a nitrogen source, an acetate source, and a pH buffer, said pH buffer resulting in a pH of said aqueous culture medium of about 5.2-7.5.” In the specification of this patent (starting at line 49 of Column 2 thereof), it was disclosed that: “A magnetotactic bacterium was isolated from fresh water swamps and was cultured in the laboratory on the special growth medium of the present invention. Frankel, Blakemore, and Wolfe, Science, 203, 1355 (1979). The organism is a magnetotactic Aquaspirillum and appears to be a new bacterial species by criteria separate from its magnetic properties. It has been designated strain MS-1. A culture of this microorganism has been deposited in the permanent collection of the American Type Culture Collection, Rockville, Md. A subculture of the microorganism may be obtained upon request. Its accession number in this repository is ATCC 31632”

U.S. Pat. No. 4,452,896 of Richard P. Blakemore et al. is another U.S. patent relating to magnetic bacteria; the entire disclosure of this U.S. patent is also incorporated by reference into this specification. This U.S. patent describes and claims: “A method for growing a biologically pure culture of magnetic bacteria, comprising mixing, per 100 ml, about 2-30 μM of ferric quinate, about 10-1000 mg. of an organic compound selected from the group consisting of fumaric acid, tartaric acid, malic acid, succinic acid, lactic acid, pyruvic acid, oxaloacetic acid, malonic acid, β-hydroxybutyric acid, maleic acid, galactose, rhamnose, melibiose, acetic acid, adipic acid, and glutaric acid, a vitamin source, a mineral source, a nitrogen source, an acetate source, and a pH buffer within the range of about 5.2-7.5, inoculating the mixture with said magnetic bacteria, providing said magnetic bacteria with an atmosphere having an initial oxygen concentration of about 0.2-6% by volume, and maintaining the ambient temperature in the range of about 18°-35° C.”

In one embodiment of this invention, magnetotactic bacteria comprised of one or more anti-microtubule agents are caused to migrate to the coated substrate assembly 3100 (see FIG. 36) by the application of an external magnetic field.

Magnetotactic bacteria migrate along the direction of a magnetic field. In one embodiment, of this invention, one or more anti-microtubule agents, such as paclitaxel (or other similar cancer drugs) are incorporated into such bacteria. One may, e.g., coat the paclitaxel with an organic material that the specific type of bacteria used will be attracted to as a nutrient and hence ingest drug molecules in the process. Subsequently, the paclitaxel-containing bacteria are directed towards the desired site in a patient's body through an application of a magnetic field as guidance for their migration to such site. In one aspect of this embodiment, paclitaxel-containing bacteria are injected into, onto, or near the desired site. In another aspect of this embodiment, the paclitaxel-containing bacteria are fed to the patient, who is then subjected to electromagnetic radiation in accordance with the procedure described elsewhere in this specification.

Thus, e.g., the electromagnetic radiation or an inhomogeneous magnetic field can be focused onto the desired site(s), in which case the magnetotactic bacterial would drift towards the tumor site and excrete the Paclitaxel at such site executing a drug delivery mechanism to the site in the process. This process would continue as long as the electromagnetic radiation continued to be applied.

It should be noted that bacteria are prokaryotic organisms that are not as adversely affected by anti-microtubule agents as are human beings in that the bacteria do not express tubulin.

Referring again to FIG. 20 of the instant specification, and to the preferred embodiment depicted therein, the morphologically indented surface 3106 may be made by conventional means.

Referring again to FIG. 20, and in one preferred embodiment thereof, the size of the indentations 3108 is preferably chosen such that it matches the size of the drug particles 3110. In one embodiment, depicted in FIG. 36A, the surface 3112 of the indentations 3108 is coated with receptor material 3114 adapted to bind to the drug particles 3110.

Receptor material 3114 is comprised of a “recognition molecule”. As is known to those skilled in the art, recognition is a specific binding interaction occurring between macromolecules.

Many recognition molecules and recognition systems are described in, e.g., United States patents.

Thus, by way of illustration, U.S. Pat. No. 5,482,836 (the entire disclosure of which is hereby incorporated by reference into this specification) discloses a process which utilizes both a “first recognition molecule of a specific molecular recognition system” and a “second recognition molecule specifically binding to the first recognition molecule.” As is disclosed in column 3 of this patent, “. . . a molecular recognition system is a system of at least two molecules which have a high capacity of molecular recognition for each other.” This term is also discussed at column 6 of U.S. Pat. No. 5,482,836, wherein it is stated that: “A ‘molecular recognition system’ is a system of at least two molecules which have a high capacity of molecular recognition for each other and a high capacity to specifically bind to each other. Molecular recognition systems for use in the invention are conventional and are not described here in detail. Techniques for preparing and utilizing such systems are well-known in the literature and are exemplified in the publication Tijssen, P., Laboratory Techniques in Biochemistry and Molecular Biology Practice and Theories of Enzyme Immunoassays, (1988), eds. Burdon and Knippenberg, New York:Elsevier.”

The terms “bind” or “bound”, etc. include both covalent and non-covalent associations, but can also include other molecular associations where appropriate such as Hoogsteen hydrogen bonding and Watson-Crick hydrogen bonding.”

At column 7 of U.S. Pat. No. 5,482,836, a description of some typical molecular recognition systems is presented. These systems include “. . . an antigen/antibody, an avidin/biotin, a streptavidinibiotin, a protein A/Ig and a lectin/carbohydrate system. The preferred embodiment of the invention uses the streptavidin/biotin molecular recognition system and the preferred oligonucleotide is a 5′-biotinylated homopyrimidine oligonucleotide.”

Thus, by way of further illustration, U.S. Pat. No. 5,705,163 describes “A method for killing a target cell, said method comprising contacting said target cell with a cytotoxic amount of a composition comprising a recombinant Pseudomonas exotoxin (PE) having a first recognition molecule for binding said target cell and a carboxyl terminal sequence of 4 to 16 amino acids which permits translocation of the PE molecule into a cytosol of said target cell, the first recognition molecule being inserted in domain III after and no acid 600 and before amino acid 613 of the PE” (see claim 1). The entire disclosure of this United States patent is hereby incorporated by reference into this specification.

Thus, by way of yet further illustration, U.S. Pat. No. 5,922,537 describes a “binding agent bound through specific recognition sites to an immobilized analyte” (see claim 1). The entire disclosure of this United States patent is hereby incorporated by reference into this specification.

Thus, by way of further illustration, U.S. Pat. No. 6,297,059 describes “An optical biosensor for detection of a multivalent target biomolecule comprising: a substrate having a fluid membrane thereon; recognition molecules situated at a surface of said fluid membrane, said recognition molecule capable of binding with said multivalent target biomolecule and said recognition molecule linked to a single fluorescence molecule and as being movable upon said surface of said fluid membrane; and, a means for measuring a change in fluorescent properties in response to binding between multiple recognition molecules and said multivalent target biomolecule” (see claim 1.). As is disclosed in column 1 of this patent, “Biological sensors are based upon the immobilization of a recognition molecule at the surface of a transducer (a device that transforms the binding event between the target molecule and the recognition molecule into a measurable signal). In one prior approach, the transducer has been sensitive to any binding, specific or non-specific, that occurred at the transducer surface. Thus, for surface plasmon resonance or any other transduction that depended on a change in the index of refraction, such sensors have been sensitive to both specific and non-specific binding. Another prior approach has relied on a sandwich assay where, for example, the binding of an antigen by an antibody has been followed by the secondary binding of a fluorescently tagged antibody that is also in the solution along with the protein to be sensed. In this approach, any binding of the fluorescently tagged antibody will give rise to a change in the signal and, therefore, sandwich assay approaches have also been sensitive to specific as well as non-specific binding events. Thus, selectivity of many prior sensors has been a problem.”

U.S. Pat. No. 6,297,059 also discloses that “Another previous approach where signal transduction and amplification have been directly coupled to the recognition event is the gated ion channel sensor as described by Cornell et al., “A Biosensor That Uses Ion-Channel Switches”, Nature, vol. 387, Jun. 5, 1997. In that approach an electrical signal was generated for measurement. Besides electrical signals, optical biosensors have been described in U.S. Pat. No. 5,194,393 by Hugl et al. and U.S. Pat. No. 5,711,915 by Siegmund et al. In the later patent, fluorescent dyes were used in the detection of molecules.” In one embodiment of the process of this invention, the binding of a specific binding pair that is facilitated by the process of this invention is sensed and reported by a biological sensor.

Thus, by way of further illustration, U.S. Pat. No. 6,337,215 (the entire disclosure of which is hereby incorporated by reference into this specification) discloses “an affinity recognition molecule attached to the coating of the magnetic particle for selectively binding with a target molecule” (see claim 1 of the patent). In particular, claim 1 of U.S. Pat. No. 6,337,215 describes: “A composition of matter comprising: a magnetic particle comprising a first ferromagnetic layer having a moment oriented in a first direction, a second ferromagnetic layer having a moment oriented in a second direction generally antiparallel to said first direction, and a nonmagnetic spacer layer located between and in contact with the first and second ferromagnetic layers, and wherein the magnitude of the moment of the first ferromagnetic layer is substantially equal to the magnitude of the moment of the second ferromagnetic layer so that the magnetic particle has substantially zero net magnetic moment in the absence of an applied magnetic field, and wherein the thickness of the magnetic particle is substantially the same as the total thickness of said layers making up the particle; a coating on the surface of the magnetic particle; and an affinity recognition molecule attached to the coating of the magnetic particle for selectively binding with a target molecule.”

The “affinity recognition molecules” of U.S. Pat. No. 6,337,215, and means for attaching them to magnetic particles, are described in columns 16-18 of such patent, wherein it is disclosed that: “The following sections discuss the use of the above identified magnetic particles as nuclei for affinity molecules that are bound to the magnetic particles of the present invention. As indicated above, magnetic particles according to the present invention are attached to at least one affinity recognition molecule. As used herein, the term ‘affinity recognition molecule’ refers to a molecule that recognizes and binds another molecule by specific three-dimensional interactions that yield an affinity and specificity of binding comparable to the binding of an antibody with its corresponding antigen or an enzyme with its substrate. Typically, the binding is noncovalent, but the binding can also be covalent or become covalent during the course of the interaction. The noncovalent binding typically occurs by means of hydrophobic interactions, hydrogen bonds, or ionic bonds. The combination of the affinity recognition molecule and the molecule to which it binds is referred to generically as a ‘specific binding pair.’ Either member of the specific binding pair can be designated the affinity recognition molecule; the designation is for convenience according to the use made of the interaction. One or both members of the specific binding pair can be part of a larger structure such as a virion, an intact cell, a cell membrane, or a subcellular organelle such as a mitochondrion or a chloroplast.” As will be apparent, one or more of such recognition molecules may be attached to the surface(s) of the nanomagnetic particles of this invention.

U.S. Pat. No. 6,337,215 also discloses-that “Examples of affinity recognition molecules in biology include antibodies, enzymes, specific binding proteins, nucleic acid molecules, and receptors. Examples of receptors include viral receptors and hormone receptors. Examples of specific binding pairs include antibody-antigen, antibodyhapten, nucleic acid molecule-complementary nucleic acid molecule, receptor-hormone, lectin-carbohydrate moiety, enzyme substrate, enzyme-inhibitor, biotin-avidin, and viruscellular receptor. One particularly important class of antigens is the Cluster of Differentiation (CD) antigens found on cells of hematopoietic origin, particularly on leukocytes, as well as on other cells. These antigens are significant in the activity and regulation of the immune system. One particularly significant CD antigen is CD34, found on stem cells. These are totipotent cells that can regenerate all of the cells of hematopoietic origin, including leukocytes, erythrocytes, and platelets.”

U.S. Pat. No. 6,337,215 also discloses that “As used herein, the term “antibody” includes both intact antibody molecules of the appropriate specificity and antibody fragments (including Fab, F(ab′), Fv, and F(ab′)2 fragments), as well as chemically modified intact antibody molecules and antibody fragments such as Fv fragments, including hybrid antibodies assembled by in vitro reassociation of subunits. The term also encompasses both polyclonal and monoclonal antibodies. Also included are genetically engineered antibody molecules such as single chain antibody molecules, generally referred to as sFv. The term “antibody” also includes modified antibodies or antibodies conjugated to labels or other molecules that do not block or alter the binding capacity of the antibody.”

U.S. Pat. No. 6,337,215 also discloses that “As used herein, the terms nucleic acid molecule,’ ‘nucleic acid segment’ or ‘nucleic acid sequence’ include both DNA and RNA unless otherwise specified, and, unless otherwise specified, include both double-stranded and single stranded nucleic acids. Also included are hybrids such as DNA-RNA hybrids. In particular, a reference to DNA includes RNA that has either the equivalent base sequence except for the substitution of uracil and RNA for thymine in DNA, or has a complementary base sequence except for the substitution of uracil for thymine, complementarity being determined according to the Watson-Crick base pairing rules. Reference to nucleic acid sequences can also include modified bases or backbones as long as the modifications do not significantly interfere either with binding of a ligand such as a protein by the nucleic acid or with Watson-Crick base pairing.”

U.S. Pat. No. 6,337,215 also discloses that “Methods for the covalent attachment of biological recognition molecules to solid phase surfaces, including the magnetic particles of the present invention, are well known in the art and can be chosen according to the functional groups available on the biological recognition molecule and the solid phase surface.”

U.S. Pat. No. 6,337,215 also discloses that “Many reactive groups on both protein and non-protein compounds are available for conjugation. For example, organic moieties containing carboxyl groups or that can be carboxylated can be conjugated to proteins via the mixed anhydride method, the carbodiimide method, using dicyclohexylcarbodiimide, and the N hydroxysuccinimide ester method.”

U.S. Pat. No. 6,337,215 also discloses that “If the organic moiety contains amino groups or reducible nitro groups or can be substituted with such groups, conjugation can be achieved by one of several techniques. Aromatic amines can be converted to diazonium salts by the slow addition of nitrous acid and then reacted with proteins at a pH of about 9. If the organic moiety contains aliphatic amines, such groups can be conjugated to proteins by various methods, including carbodiiumide, tolylene-2,4-diisocyanate, or malemide compounds, particularly the N-hydroxysuccinimide esters of malemide derivatives. An example of such a compound is 4(Nmaleimidomethyl)-cyclohexane-1-carboxylic acid. Another example is m-male imidobenzoyl-N-hydroxysuccinimide ester. Still another reagent that can be used is N-succinimidyl-3 (2-pyridyldithio) propionate. Also, bifunctional esters, such as dimethylpimelimidate, dimethyladipimidate, or dimethylsuberimidate, can be used to couple amino-group containing moieties to proteins.”

U.S. Pat. No. 6,337,215 also discloses that “Additionally, aliphatic amines can also be converted to aromatic amines by reaction with p-nitrobenzoylchloride and subsequent reduction to a p-aminobenzoylamide, which can then be coupled to proteins after diazotization.”

U.S. Pat. No. 6,337,215 also discloses that “Organic moieties containing hydroxyl groups can be cross-linked by a number of indirect procedures. For example, the conversion of an alcohol moiety to the half ester of succinic acid (hemisuccinate) introduces a carboxyl group available for conjugation. The bifunctional reagent sebacoyldichloride converts alcohol to acid chloride which, at pH 8.5, reacts readily with proteins. Hydroxyl containing organic moieties can also be conjugated through the highly reactive chlorocarbonates, prepared with an equal molar amount of phosgene.”

U.S. Pat. No. 6,337,215 also discloses that “For organic moieties containing ketones or aldehydes, such carbonyl-containing groups can be derivatized into carboxyl groups through the formation of O-(carboxymethyl) oximes. Ketone groups can also be derivatized with p-hydrazinobenzoic acid to produce carboxyl groups that can be conjugated to the specific binding partner as described above. Organic moieties containing aldehyde groups can be directly conjugated through the formation of Schiff bases which are then stabilized by a reduction with sodium borohydride.”

U.S. Pat. No. 6,337,215 also discloses that “One particularly useful cross-linking agent for hydroxyl-containing organic moieties is a photosensitive noncleavable heterobifunctional cross-linking reagent, sulfosuccinimidyl 6-[4-azido-2-nitrophenylamino] hexanoate. Other similar reagents are described in S. S. Wong, “Chemistry of Protein Conjugation and CrossLinking,” (CRC Press, Inc., Boca Raton, Fla. 1993). Other methods of crosslinking are also described in P. Tijssen, “Practice and Theory of Enzyme Immunoassays” (Elsevier, Amsterdam, 1985), pp. 221-295.”

U.S. Pat. No. 6,337,215 also discloses that “Other cross-linking reagents can be used that introduce spacers between the organic moiety and the biological recognition molecule. The length of the spacer can be chosen to preserve or enhance reactivity between the members of the specific binding pair, or, conversely, to limit the reactivity, as may be desired to enhance specificity and inhibit the existence of cross-reactivity.”

U.S. Pat. No. 6,337,215 also discloses that “Although, typically, the biological recognition molecules are covalently attached to the magnetic particles, alternatively, noncovalent attachment can be used. Methods for noncovalent attachment of biological recognition molecules to magnetic particles are well known in the art and need not be described further here.”

U.S. Pat. No. 6,337,215 also discloses that “Conjugation of biological recognition molecules to magnetic particles is described in U.S. Pat. No. 4,935,147 to Ullman et al., and in U.S. Pat. No. 5,145,784 to Cox et al., both of which are incorporated herein by this reference.”

Referring to FIGS. 1 and 1A, one may bind biological recognition molecules to the container 12 and/or the nanomagnetic film 16 and/or the polymeric material 14 by the means disclosed in U.S. Pat. No. 6,337,215.

Thus, by way of further illustration, U.S. Pat. No. 6,682,648 describes “a recognition molecule capable of specifically binding an analyte in a structure restricted manner” (see claim 1); the entire disclosure of this United States patent is hereby incorporated by reference into this specification. The “analyte” disclosed in such patent is preferably an antigen or antibody. Thus, as is disclosed at column 7 of this patent, “The term “antibody” refers to immunoglobulins of any isotype or subclass as well as any fab or fe fragment of the aforementioned. Antibodies of any source are applicable including polyclonal materials obtained from any animal species; monoclonal antibodies from any hybridoma source; and all immunoglobulins (or fragments) generated using viral, prokaryotic or eukaryotic expression systems. Biologic recognition molecules other than antibodies, are equally applicable for use with the current invention. These include, but are not limited to: cell adhesion molecules, cell surface receptor molecules, and solubilized binding proteins. Non-biologic binding molecules, such as ‘molecular imprints’ (synthetic polymers with pre-determined specifically for binding/complex formation), are also applicable to the invention. The terms ‘antigens,’ ‘immunogens’ or ‘haptens’ refer to substances which can be recognized by in vivo or in vitro immune elements, and are capable of eliciting a cellular or humoral immunologic response.” Although the electrochemically active reporter utilized in the embodiment is specified as para-aminophenol (generated by the action of a beta-galactosidase conjugate in conjunction with a specific substrate), it should be noted that the invention is generally applicable to molecules capable of redox recycling, and enzyme systems capable of generating such reporters.”

Thus, by way of illustration, U.S. Pat. No. 6,686,209 discloses a recognition molecule having a binding site that is capable of binding to tetrahydrocannabinoids. The entire disclosure of this United States patent is hereby incorporated by reference into this specification.

By way of further illustration, “recognition molecules” and/or “recognition systems” and/or “affinity molecules” and/or “specific binding pairs” are disclosed, e.g., in U.S. Pat. Nos. 5,268,306 (preparation of a solid phase matrix containing a bound specific pair), 6,103,537 (separation of free and bound species), 5,972,630, 6,399,299, 6,261,554 (compositions for targeted gene delivery), 6,054,281 (binding assays), 6,004,745 (hybridization protection assay), 5,998,192, 5,851,770 (detection of mismat ches by resolvase cleavage using a magentic bead support), 5,716,778 (concentrating immunochemical test device), 5,639,604 (homogeneous protection assay), 4,629,690 (homogeneous enzyme specific binding assay on non porous surface), 4,435,504, 6,489,123 (labelling and selection of molecules), 6,342,588, 6,180,336, 6,1543,442 (reagents and methods for specific binding assays), 6,068,981 (marking of orally ingested products), 5,8538,983 (inhibition of cell adhesion protein-carbohydrate interactions), 5,801,000 (detection and isolation of receptors), 5,766,934 (sensors with immobilized indicator molecules), 5,554,499 (detection and isolation of ligands), 4,713,350 (hydrophilic assay containing one member of a specific binding pair), 4,650,751 (protected binding assay), 4,575,485 (ultasonic ehanced immuno-reactions), and the like. The entire disclosure of each of these United States patents is hereby incorporated by reference into this specification. One may bind one or more of these recognition molecules to the container 12 and/or the polymeric material 14 and/or the nanomagentic material 16 by one or more of the means disclosed in such patents.

Referring again to FIG. 20, and in the embodiment depicted, an external electromagnetic field 3116 is shown being applied near the surface 3106 of the coated substrate 3100. In the embodiment depicted, this applied field 3116 is adapted to facilitate the bonding of the drug particles 3110 to the indentations 3108. As long as such indentations are not totally filled, and as long as the appropriate electromagentic field is applied, then the drug molecules 3110 will continue to bond to such indentations 3108. In one embodiment, not depicted in FIG. 20, instead of drug particles 3110 or in addition thereto, one or more of the nanomagnetic particles of this invention may be caused to bind to a specific site within a biological organism.

The external attachment electromagnetic field 3116 may, e.g., be ultrasound. It is known that ultrasound can be used to greatly enhance the rate of binding between members of a specific binding pair. Reference may be had, e.g., to U.S. Pat. No. 4,575,485, which claims:” In a method for measuring the binding of members of a specific binding pair in an aqueous medium, the improvement which comprises ultrasonicating the medium containing the members of the specific binding pair for a sufficient time to enhance the rate of binding of said members” (see claim 1). As is disclosed in this patent, improved “. . . rates are obtained in the binding between members of a specific binding pair, particularly where one of the members of the specific binding pair is bound to a solid support . . . . ” The entire disclosure of this United States patent is hereby incorporated by reference into this specification.

As is further disclosed in U.S. Pat. No. 4,575,485, “As mentioned above, of particular interest for the subject invention is where one of the members of the specific binding pair is conjugated to a solid support, usually non-diffusibly conjugated to a non-dispersible solid support . . . . The specific binding member may be conjugated to the support either covalently or non-covalently, normally depending upon the specific member, as well as the nature of the support.”

U.S. Pat. No. 4,575,485 also discloses that “To enhance the rate of reaction of the ligand and receptor to form the complex in an assay such as one described above, the assay medium may be subjected to ultrasonication such as by introduction into a bath in an ultrasonic device. Generally, the medium is subjected to ultrasonic sound for a time sufficient to allow for at least about 25% of the binding between the members of the specific binding pair to occur. The frequency of ultrasonication will vary from about 5 to 103 kHz, preferably from about 15 to 500 kHz, depending upon the size of the bath, the time for the ultrasonication, and the available equipment. The power will generally be from about 10 to 100 watts, more usually from about 25 to 75 watts, and preferably from about 45 to 60 watts. The temperature will generally be maintained in the range of about 150 to 40° C. The assay medium will generally be a volume in the range of about 0.1 ml to 10 ml, usually from about 0.1 ml to 5 ml. The time may vary, depending on the frequency and power, from about 30 seconds to 2 hours, more usually from about 1 minute to 30 minutes. The power, frequency, and time will be chosen so as not to have a deleterious effect on the binding members and to assure accuracy of the assay.”

As is known to those skilled in the art, paclitaxel, and paclitaxel-type compounds, stabilize microtubules, preventing them from shortening and dividing the cell as a result of their shortening as they segregate the genetic material in chromosomes. Furthermore, paclitaxel increases the rigidity of microtubules making them susceptible to breaking given the right physical stimuli.

Ultrasound induces mechanical vibrations of microtubules. At the right frequency, and at the right power level, the application of ultrasound will cause the microtubules to first buckle and then break up.

The ultrasound used in one embodiment of the process of this invention preferably has a frequency of from about 50 megahertz to about 2 Gigahertz, and more preferably has a frequency of from about 100 megahertz to about 1 Gigahertz. The power of such ultrasound is preferably at least about 0.01 watts per square meter and, more preferably, at least about 0.1 watts per square meter. The ultrasound is preferably focused on the site to be treated, such as, e.g., a tumor. One may use any conventional means for focusing the ultrasound. Thus, e.g., one may use one or more of the devices disclosed in U.S. Pat. Nos. 6.613,0055 (systems and methods for steering a focused ultrasound array), 6,613,004, 6,595,934 (skin rejuvenation using high intensity focused ultrasound), 6,543,272 (calibrating a focused ultrasound array), 6,506,154 (phased array focused ultrasound system), 6,488,639 (high intensity focused ultrasound treatment apparatus), 6,451,013 (tonsil reduction using high intensity focused ultrasound to form an ablated tissue area), 6,432,067 (medical procedures using high-intensity focused ultrasound), 6,425,867 (noise-free real time ultrasonic imaging of a treatment site undergoing high intensity focused ultrasound therapy), and the like. The entire disclosure of each of these patent applications is hereby incorporated by reference into this specification.

In one embodiment, paclitaxel (or a similar composition) is delivered to the patient and, as is its wont, makes the microtubules more rigid. Thereafter, when the microtubules are polymerized in a dividing cell and substantially immobilized, the ultrasound is selectively delivered to the microtubules in delivery site, thereby breaking such microtubules and halting the process of cell growth.

In one aspect of this embodiment, after the paclitaxel (or similar material) has been delivered to the patient, the high intensity magnetic field is applied to the delivery site in order to selectively cause the paclitaxel to bind the microtubules in the site. Thereafter, the ultrasound is applied to break the microtubules so bound to the Paclitaxel enhancing the efficacy of the drug due to a combined effect of the magnetic field, ultrasound and chemotherapeutic action of Paclitaxel itself.

When microtubules have been broken, they tend to reform. Therefore, in one embodiment, the ultrasound is periodically or continuously delivered to the delivery site synchronized to the typical time elapsed between subsequent cell division processes during which microtubules are polymerized.

In one embodiment, not shown, a portable device is worn by the patient; and this device periodically and/or continuously delivers ultrasound and/or magnetic energy to the patient. In one aspect of this embodiment, the device first delivers high intensity magnetic energy, and then it delivers the ultrasound energy.

As is known to those skilled in the art, ultrasound is by one of the many forms of electromagnetic radiation that affect biological processes in general and, in particular, may affect the rate of binding or disassociation between two members of a specific binding pair. Some of these forms of electromagnetic radiation are disclosed in columns 2-4 of U.S. Pat. No. 5,566,685, the entire disclosure of which is hereby incorporated by reference into this specification. As is disclosed in this patent, at columns 1-2 thereof, “The prevalence of ELF EMFs at home, in educational establishments and in the work place, where people spend a great deal of their time, has for the past 10 years fueled considerable interest in scientific research to examine the possibility of adverse health effects from exposure to these fields. At the present time overwhelming evidence exists which shows that a wide range of biological effects are possible even at very low levels of exposure (<5 milligauss-mG). These effects include changes in transcription of specific genes, changes in enzyme activities, production of morphological abnormalities and biochemical modifications in developing chick embryos, stimulation of bone cell growth, suppression of nocturnal melatonin in humans, and alterations in cellular Ca2+ pools [Goodman, R., L.-X. Wei, J.-C. Xu, and A. Henderson, ‘Exposure of human cells to low-frequency electromagnetic fields results in quantitative changes in transcripts’, Biochim. Biophys. Acta, 1009:216-220, 1989; Battini, R., M. G. Monti, M. S. Moruzzi, S. Ferrari, P. Zaniol, and B. Barbiroli, ‘ELF electromagnetic fields affect gene expression of regenerating rat liver following partial hepatectomy’, J. Bioelec. 10:131-139, 1991; Krause, D., W. J. Skowronski, J. M. Mullins, R. M. Nardone, and J. J. Greene ‘Selective enhancement of gene expression by 60 Hz electromagnetic radiation’, in C. T. Brighton and S. R. Pollack, Eds. ‘Electromagnetics in Biology and Medicine’ (San Francisco Press, Inc., San Francisco, Calif.) pp. 133-138, 1991; Phillips, J. L., W. Haggren, W. J. Thomas, T. Ishida-Jones, and W. R. Adey, ‘Magnetic field-induced changes in specific gene transcription’, Biochim. Biophys. Acta 1132:140-144, 1992; Greene, J. J., S. L. Pearson, W. J. Skowronski, R. M. Nardone, J. M. Mullins, and D. Krause, ‘Gene-specific modulation of RNA synthesis and degradation by extremely low frequency electromagnetic fields’, Cell. Mol. Biol. 39:261-268, 1993; Byus, C. V., R. L. Lundak, R. M. Fletcher, and W. R. Adey, ‘Alterations in protein kinase activity following exposure of cultured human lymphocytes to modulated microwave fields’, Bioelectromag. 5:341-351, 1984; Byus, C. V., S. E. Pieper, and W. R. Adey, ‘The effects of low-energy 60-Hz environmental electromagnetic fields upon the growth-related enzyme ornithine decarboxylase’, Carcinogenesis 8:1385-1389, 1987; Litovitz, T. A., D. Krause, and J. M. Mullins, ‘Effects of coherence time of the applied magnetic field on ornithine decarboxylase activity’, Biochem. Biophys. Res. Commun. 178:862-865, 1991; Litovitz, T. A., D. Krause, M. Penafiel, E. C. Elson, and J. M. Mullins, ‘The role of coherence time in the effect of microwaves on ornithine decarboxylase’, Bioelectromagnetics 14:395-403, 1993; Monti, M. G., L. Pernecco, M. S. Moruzzi, R. Battini, P. Zaniol, and B. Barbiroli, ‘Effect of ELF pulsed electromagnetic fields on protein kinase C activation process in HL-60 leukemia cells’, J. Bioelec. 10:119-130, 1991; Blank, M., ‘Na K-ATPase function in alternating electric fields’, FASEB J. 6:2434-2438, 1992; Delgado, J. M. R., J. Leal, J. L. Monteagudo, and M. G. Garcia, ‘Embryological changes induced by weak, extremely low frequency electromagnetic fields’, J. Anat. 134:533-551, 1992; Juutilainen, J., E. Laara, and K. Saali, ‘Relationship between field strength and abnormal development in chick embryos exposed to 50 Hz magnetic fields’, Int. J. Radiat. Biol. 52:787-793, 1987; Martin, A. H., ‘Magnetic fields and time dependent effects on development’, Bioelectromagnetics 9:393-396, 1988; Aaron, R., D. Ciombor, and G. Jolly, ‘Stimulation of experimental endochondral ossification by low-energy pulsing electromagnetic fields’, J. Bone Mineral Res. 4:227-233, 1989; Bassett, C. A. L., ‘Beneficial effects of electromagnetic fields’, J. Cell. Biochem. 51:387-393, 1993; Ciombor, D. M., and R. K. Aaron, ‘Influence of electromagnetic fields on endochondral bone formation’, J. Cell. Biochem. 52:37-41, 1993; Graham, C., M. R. Cook, H. D. Cohen, D. W. Riffle, S. J. Hoffman, F. J. McClernon, D. Smith, and M. M. Gerkovich, ‘EMF suppression of nocturnal melatonin in human volunteers, Abstract in the Proceedings of the Department of Energy Contractors Review Meeting October 1993; Wilson B. W., Wright C. W., Morris J. E., Buschbom R. L., and others ‘Evidence for an effect of ELF electromagnetic fields on human pineal gland function’, J. Pineal Res. 9:259-69, 1990; Reiter R. J., Anderson L. E., Busschbom R. L., Wilson B. W., ‘Reduction of the nocturnal melatonin rise in rats exposed to 60 Hz electric fields in utero and for 23 days after birth’, Life Sci. 42:2203-2206, 1988; Bawin, S. M., and W. R. Adey, ‘Sensitivity of calcium binding in cerebral tissue to weak environmental electric fields oscillating at low frequency’, Proc. Natl. Acad. Sci. USA 73:1999-2003, 1976; Bawin, S. M., W. R. Adey, and I. M. Sabbot, ‘Ionic factors in release of Ca2+ from chicken cerebral tissue by electromagnetic fields’, Proc. Natl. Acad. Sci. USA 75:6314-6318, 1978; Blackman, C. F., S. G. Benane, L. S. Kinney, D. E. House, and W. T. Joines, ‘Effects of ELF fields on calcium-ion efflux from brain tissue, in vitro’, Radiat. Res. 92:510-520, 1982; Lindstrom, E., P. Linstrom, A. Berglund, K. H. Mild, and E. Lundgren, ‘Intracellular calcium oscillations induced in a T-cell line by a weak 50 Hz magnetic field’, J. Cell. Physiol. 156:395-398 1993].”

A recent article by J. Ratoff appeared in “Science News” (published by Science Service, 1719 N. Street, N.W., Washington, D.C. 20036. This article, entitled “Magnetic Fields can diminish drug action,” disclosed that “The low-level electromagnetic fields present in some North American homes today can diminish or wipe out a wide prescribed drug's actions . . . . Researcher's have found that, when exposed to such fields, the drug tamoxifen lost its ability to halt the proliferation of cancer cells . . . . Gamoxifen is a synthetic hormone used to prevent the recurrence of breast cancer.”

A Jul. 3, 1993 article in “Science News” (see page 10 thereof) reported research that showed that while melatonin, a natural antioxidant hormone, would inhibit the growth of breast cancer cells exposed to 2 milligauss magnetic fields, its activity was essentially reased when the cells were based in a 12 milliGauss field.

Articles on similar subjects have been published by: Blackman, C. F., et al., 1996, “Independent replication of the 12-mg magnetic field effect on melatonin and mcf-7 cells in vitro,” Eighteenth annual meeting of the Bioelectromagnetic Society, Victoria, British, Columbia; Harland, J. D. and R. P. Liburdy, 1997, “Environmental magnetic fields inhibit the antiproliferative action of tamoxifen and melatonin in a human breast cancer cell line,” Bioelectromagnetics 18; and Liburdy, R. P., et al., 1997, “A 12 mG . . . magnetic field inhibits tamoxifen's oncostatic action in a second human breast cancer cell line,: T47D, Second World Congress for Electricity and Magnetism in Biology and Medicine, Bologna, Italy.

Related articles appearing in “Science News” include, e.g., “EMFs on the brain?,” Science News 147 (Jan. 21, 1995):44; “Study reaffirms tamoxifen's dark side,” Science News 145 (Jun. 4, 1994): 356; “Cells haywire in electromagnetic field?,” Science News 133 (Apr. 2, 2988):216, “Power-line static,” Science News 140 (Sep. 28, 1991): 202; and “Do EMFs pose breast cancer risk?,” Science News 145 (Jun. 18, 1994): 388.

In one embodiment, the electromagnetic radiation used in the process of this invention is a magnetic field with a field strength of at least about 6 Tesla. It is known, e.g., that microtubules move linearly in magnetic fields of at least about 6 Tesla.

In this embodiment, the focusing of the magnetic field onto an in vivo site within a patient may be done by conventional magnetic focusing means. Thus, and referring to U.S. Pat. No. 5,929,732 (the entire disclosure of which is hereby incorporated by reference into this specification), one may utilize: “An apparatus and method for creating a magnetic beam wherein a focusing magnet assembly (45) is comprised of a first opposing magnet pair (20) and a second opposing magnet pair (30) disposed in a focusing plane, each magnet of the respective opposing magnet pairs having a like pole directed towards the geometric center of the focusing magnet assembly (45) to form an alignment path, two like magnetic beams extending from the alignment path on each side of the focusing magnet assembly (45), each beam being generally perpendicular to the focusing plane. A like pole of an unopposed magnet (10) can be directed down the alignment path from one side of the focusing magnet assembly (45) to produce a single magnetic beam extending generally perpendicular from the focusing magnet assembly opposite unopposed magnet (10). This beam is a magnetic monopole which emits pulses, levitates, degausses, stops electronics and separates materials.”

By way of further illustration, one may use the “Permanent Magnetic Keeper-Shield Assembly” disclosed in U.S. Pat. No. 6,488,615; the entire disclosure of this United States patent application is hereby incorporated by reference into this specification. This patent discloses: “A magnet keeper-shield assembly adapted to hold and store a permanent magnet used to generate a high gradient magnetic field. Such a field may penetrate into deep targeted tumor sites in order to attract magnetically responsive micro-carriers. The magnet keeper-shield assembly includes a magnetically permeable keeper-shield with a bore dimensioned to hold the magnet. A screw driven actuator is used to push the magnet partially out of the keeper-shield. The actuator is assisted by several springs extending through the base of the keeper-shield.”

Without wishing to be bound to any particular theory, applicants believe that the use of the high intensity magnetic field(s) focused onto or into a desired site will attract paclitaxel molecules to the site of the tumor. Paclitaxel is comprised of a 6-member aromatic ring and, thus, will have an induced magnetic moment when subjected to an external field as a result of the magnetically induced electron currents in the ring. Without wishing to be bound to any particular theory, applicants believe that, in the presence of a magnetic field, a magnetic moment is induced in the paclitaxel molecule. This effect will enhance the docking and binding of the paclitaxel molecule to the nearest tubulin molecule in a microtubule.

In one embodiment, after a patient has taken paclitaxel, he is exposed to the focused magnetic radiation for at least about 30 minutes, and this process is repeated at least once a week.

It is known that paclitaxel has an inherent magnetic moment. It is also known that paclitaxel may be chemically fixed to magnetic particles that are relatively large with respect to paclitaxel molecules, that is, equivalent to or larger than individual paclitaxel molecules. Nanomagnetic particles that are substantially smaller than paclitaxel molecules, such as the nanomagnetic particles of this invention, may be chemically bound to the drug. For all of the above described methods of binding, the result is a chemical agent that will bind to tubulin and thus effect a cellular therapy for, e.g., cancer, wherein the chemical agent may also be manipulated in a magnetic field. While this disclosure will relate largely to the use of paclitaxel as a chemotoxin, the approach may be extended to any other drug or chemical therapy wherein a large contrast in uptake between tissues and/or body regions is preferred.

FIG. 20B is a schematic of an electromagnetic coil set 3160 and 3162, aligned to an axis 3164, and which in combination create a magnetic standing wave 3166. The excitation energy delivered to the two coils 3160 and 3162 comprises a set of high frequency sinusoidal signals that are determined via well known Fourier techniques, to create a first zone 3168 having a positive standing wave magnetic field ‘E’, a second zone 3170 having a zero or near-zero magnetic field, and a third zone 3172 having a positive magnetic field ‘E’. It should be noted that the two zones 3168 and 3172 need not have exactly matched waveforms, in frequency, phase, or amplitude; it is sufficient that the magnetic fields in both are large with respect to the near-zero magnetic field in zone 3170. The fields in zones 3168 and 3172 may be static standing wave fields or time-varying standing waves. It should be noted that in order to create a zone 3170 of useful size (1 to 5 cm at the lower limit) and having reasonably sharp ‘edges’, the frequencies of the Fourier waveforms used to create standing wave 3166 may be in the gigahertz range. These fields may be switched on and off at some secondary frequency that is substantially lower; the resulting switched-standing-wave fields in zones 3168 and 3172 will impart vibrational energy to any magnetic materials within them, while the near-zero switched field in zone 3170 will not impart substantial energy into magnetic materials within its boundaries. This secondary switching frequency may be adjusted in concert with the amplitude of the standing wave field to tune the vibrational energy to impart an optimal level of thermal energy to a specific molecule (e.g. paclitaxel) by virtue of the natural resonant frequency of that molecule. The energy imparted to an individual molecule will follow the relationship E_{T}=C×M×A×F², where E_{T} is the thermal energy imparted to an individual molecule, C is a constant, M is the magnetic moment of the molecule and any bound magnetic particles, A is the amplitude of the time-varying magnetic field, and F is the frequency of field switching.

FIG. 20C is a three-dimensional schematic showing the use of three sets of magnetic coils arranged orthogonally. Each of the axes, ‘X’, ‘Y’, and ‘Z’ will impart either positive thermal energy (E) in its outer zones that correspond to zones 3168 and 3172 (from FIG. 20B), or zero thermal energy, in its central zone which corresponds to zone 3170 (from FIG. 20B). It may be seen from FIG. 20C that there will be a small volume at the centroid of the overall 3-D volume that will have overall zero magnetically-induced thermal energy. The notations ‘1×E’, ‘2×E’, and ‘3×E’ denote the relative magnetically-induced thermal energy in other regions. Since the overall volume is made up of three zones in each of three dimensions, the overall volume will have 27 sectors. Of these sectors one (the centroid) will have near-zero magnetically-induced thermal energy, (6) sectors will have a ‘1×E’ energy level, (12) sectors will have a ‘2×E’ energy level, and (8) sectors will have a ‘3×E’ energy level.

If the energy imported to any individual molecule (e.g. paclitaxel bound to one or more nanomagnetic particles) is sufficiently larger than the binding energy of that molecule to its target (e.g. tubulin in the case of paclitaxel) to account for thermal losses in coupling magnetically-induced energy into the molecule, then binding between the paclitaxel molecule and the tubulin target will not occur. Thus if we define the binding energy between the two (e.g. paclitaxel to tubulin) as E_{B}, and D as a constant that compensates for damping losses due to a molecule that is not purely elastic, then the equation E_{T}>D×E_{B} will have been satisfied, and chemical binding (in this case between paclitaxel and tubulin) will not occur.

In one embodiment, a device having matched coil sets as shown in FIG. 20B, but in three orthogonal axes, creates an overall operational volume that imparts an relatively low energy in the above-described centroid (E_{T}<D×E_{B}), and imparts a relatively higher energy in the other surrounding (26) segments (E_{T}>D×E_{B}); and if the centroid volume corresponds to the site under treatment, then a high degree of binding will occur in the centroid and no binding will occur in the exterior regions. The size of the non-binding centroid region may be adjusted via alterations to the Fourier waveforms, relative energy levels may be adjusted via amplitude and frequency of field switching, and the region may be aligned to correspond to the volume of the tumor under treatment. One preferred method for use is to place the patient in the device as disclosed herein, administer either native paclitaxel (or other drug having an innate magnetic characteristic) or magnetically-enhanced Paclitaxel (nanomagnetic or other magnetic particles either chemically or magnetically bound), maintain the patient in the controlled fields for a period of time necessary for the drug to pass out of the patient's excretory system, and then remove the patient from the device.

In another embodiment, the three fields in the X, Y, and Z directions are selectively activated and deactivated in a predetermined pattern. For example, one may activate the field in the X axis, thus causing the therapeutic agent to align with the X axis. A certain time later the field along the X axis is deactivated and the field corresponding to the Y axis is activated for a predetermined period of time. The agent then aligns with the new axis. This may be repeated along any axis. By rapidly activating and deactivating the respective fields in a predetermined pattern, one imparts thermal and/or rotational energy to the molecule. When the energy imparted to the therapeutic agent is greater than the binding energy necessary to bring about a biological effect, such binding is drastically reduced.

In another embodiment, the Fourier techniques are selected so as to create a near-zero magnetic field zone external to the tissue to be treated, while a time-varying standing wave is generated within the centroid region. A therapeutic agent that is weakly attached to a magnetic carrier particle (a carrier-agent complex) is introduced into the body. In one embodiment, the carrier particle acts to inhibit the biological activity of the therapeutic agent. When the carrier-agent complex enters the region of variable magnetic field located at the centroid, the thermal energy imparted to the carrier-agent complex the agent is liberated from its carrier and is no longer inhibited by the presence of that carrier. The region external to the centroid is a near-zero magnetic field, thus minimizing any premature dissociation of the carrier-agent complex.

In one embodiment the carrier particles are organic moieties that are covalently attached to the therapeutic agent. By way of illustration and not limitation, one may covalently attach a nitroxide spin label to a therapeutic agent. As is know to those skilled in the art, a nitroxide spin label is a persistent paramagnetic free radical. Biomolecules are routinely modified by the attachment of such labeling compounds, thus generating paramagnetic biomolecules. Reference may be had to U.S. Pat. No. 6,271,382, the entire disclosure of which is hereby incorporated by reference into this specification.

In another embodiment the carrier particles are magnetic encapsulating agents that surround the therapeutic agent. By way of illustration and not limitation, one may encapsulate a therapeutic agent within magnetosomes or magnetoliposomes described elsewhere in this specification. The agent exhibits minimal biological activity when in a near-zero magnetic field as the agent is at least partially encapsulated. When the carrier-agent complex is exposed to a variable magnetic field of sufficient intensity, the carrier particle releases the agent at or near the desired location.

Referring again to FIGS. 20 and 36A, it will be seen that FIG. 20A is a partial sectional view of an indentation 3108 coated with a multiplicity of receptors 3114 for the drug molecules.

FIG. 21 is a schematic illustration of one process for preparing a coating with morphological indentations 3108. In this process, a mask 3120 is disposed over the film 3014. The mask 3120 is comprised of a multiplicity of holes 3122 through which etchant 3124 is applied for a time sufficient to create the desired indentations 3108

One may use conventional etching technology to prepare the desired indentations 3108.

By way of illustration and not limitation, one may use the process described in claim 23 of U.S. Pat. No. 4,252,865 to prepare a surface with indentations 3108; the entire disclosure of this United States patent is hereby incorporated by reference into this specification. Claim 23 of this patent describes “The method of making a highly solar-energy absorbing surface on a substrate body, which comprises the controlled sputtering application of a layer of amorphous semiconductor material to an exposed-surface area of said body, and then altering the exposed-surface morphology of said layer by etching the same to form an array of outwardly projecting structural elements, the etchant being selected for the particular semiconductor material and applied in such strength and for such exposure time and ambient conditions of temperature as to form said structural elements with an aspect ratio in the range 2:1 to 10:1 and at lateral spacings which are in the order of magnitude of a wavelength within the solar-energy spectrum.”

By way of further illustration, one may prepare a surface with the “unique surface morphology” described in claim 1 of U.S. Pat. No. 4,233,107, the entire disclosure of which is hereby incorporated by reference into this specification. This claim 1 describes “A method of producing an ultra-black coating, having an extremely high light absorption capacity, on a substrate, the blackness being associated with a unique surface morphology consisting of a dense array of microscopic pores etched into the surface, said method comprising: (a) preparing a substrate for plating with a nickel-phosphorus alloy; (b) immersing the thus-prepared substrate in an electroless plating bath containing nickel and hypophosphite ions in solution until an electroless nickel-phosphorus alloy coating has been deposited on said substrate; (c) removing the resulting substrate with the electroless nickel-phosphorus alloy coated thereon from the plating both and washing and drying it; (d) immersing the dried substrate with the electroless nickel-phosphorus alloy coated thereon obtained in step (c) in an etchant bath consisting of an aqueous solution of nitric acid wherein the nitric acid concentration ranges from a 1:5 ratio with distilled or de-ionized water to concentrated, until the substrate surface develops ultra-blackness, said ultra-blackness being associated with said uniqud morphology; and (e) washing and drying the resulting substrate covered with the nickel-phosphorus alloy coating having said ultra-black surface.”

By way of yet further illustration, one may use the texturing process described in U.S. Pat. No. 5,830,793 and claimed in, e.g., claim 1 thereof. As is described in such claim 1, such texturing process comprises the steps of “. . . seeding a semiconductor surface adjacent a substrate surface; annealing the seeded surface; and removing seeding formations from the substrate surface, wherein seeding comprises inducing nucleation sites in a greater amount on the semiconductor surface than on the substrate surface, and removing seeding formations from the substrate surface comprises selectively etching the substrate surface relative to the semiconductor surface.”

Referring again to FIG. 21, and to the process depicted therein, after the indentations 3108 have been formed, the etchant is removed from the holes 3122 and the indentations 3108 by conventional means, such as, e.g., by rinsing, and then receptor material 3114 is used to form the receptor surface. The receptor material 3114 may be deposited within the indentations by one or more of the techniques described elsewhere in this specification.

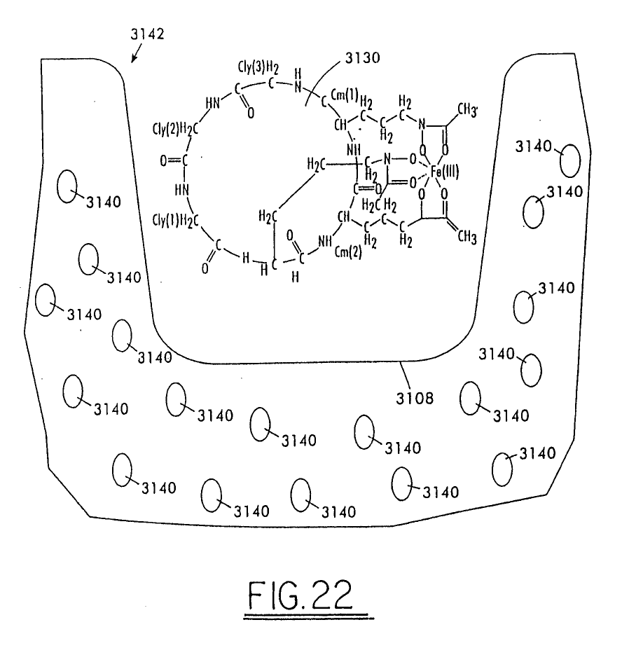

FIG. 22 is a schematic illustration of a drug molecule 3130 disposed inside of a indentation 3108. Referring to FIG. 22, and to the preferred embodiment depicted therein, it will be seen that a multiplicity of nanomagnetic particles 3140 are disposed around the drug molecule 3130. In the embodiment depicted, the forces between particles 3140 and 3130 may be altered by the application of an external field 3142. In one case, the characteristics of the field are chosen to facilitate the attachment of the particles 3130 to the particles 3140. In another case, the characteristics of the field are chosen to cause detachment of the particles 3130 from the particles 3140.

In one embodiment, the drug molecule 3130 is an anti-microtubule agent. Thus, and referring to U.S. Pat. No. 6,333,347 (the entire disclosure of which is hereby incorporated by reference into this specification), the anti-microtubule agent is preferably administered to the pericardium, heart, or coronary vasculature.

As is known to those skilled in the art, most physical and chemical interactions are facilitated by certain energy patterns, and discouraged by other energy patterns. Thus, e.g., electromagnetic attractive force may be enhanced by one applied electromagnetic filed, and electromagnetic repulsive force may be enhanced by another applied electromagnetic field. One, thus, by choosing the appropriate field(s), can determine the degree to which the one recognition molecule will bind to another, or to which a drug will bind to a implantable device, such as, e.g., a stent.

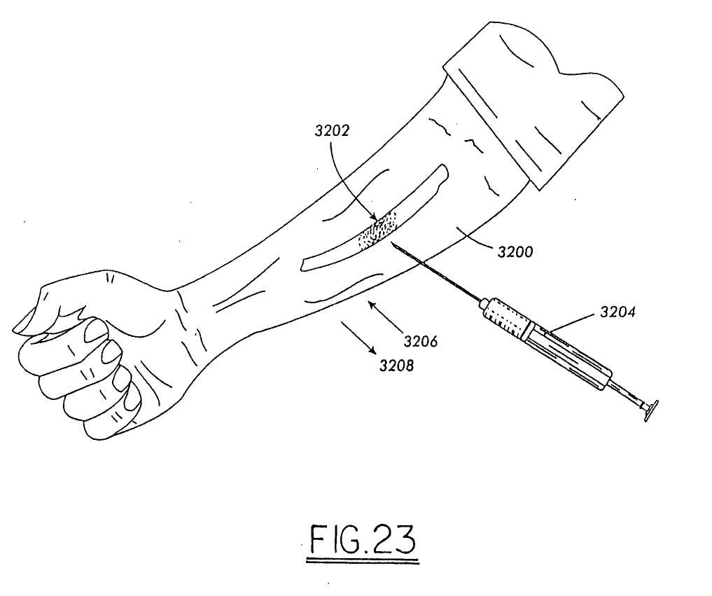

In one process, illustrated in FIG. 23, paclitaxel is administered into the arm 3200 of a patient near a stent 3202, via an injector 3204. During this administration, a first electromagnetic field 3206 is directed towards the stent 3202 in order to facilitate the binding of the paclitaxel to the stent. When it has been determined that a sufficient amount of paclitaxel has bound to the stent, a second electromagnetic field 3208 is directed towards the stent 3202 to discourage the binding of paclitaxel to the stent. The strength of the second electromagentic field 3208 is sufficient to discourage such binding but not necessarily sufficient to dislodge paclitaxel particles already bound to the stent and disposed within indentations 3208.

A Preferred Binding Process

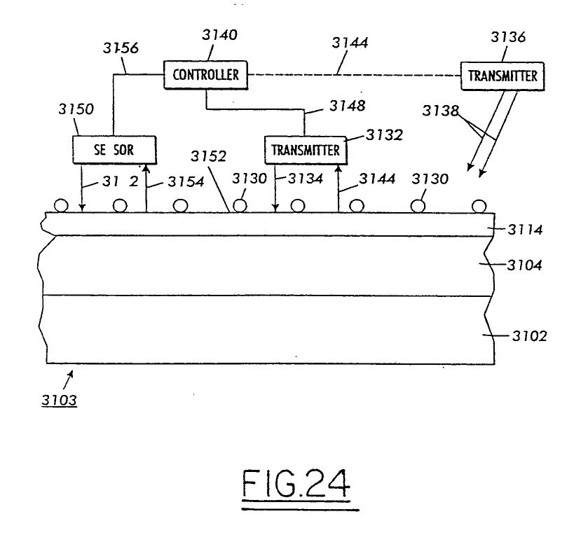

FIG. 24 is a schematic illustration of a preferred binding process of the invention. As will be apparent, FIG. 24 is not drawn to scale, and unnecessary detail has been omitted for the sake of simplicity of representation.

In the first step of the process of FIG. 24, a multiplicity of drug particles, such as drug particles 3130, are brought close to or contiguous with a coated substrate 3103 comprised of receptor material 3114 disposed on its top surface. The drug particles 3130 are near and/or contiguous with the receptor material 3114. They may be delivered to such receptor material 3114 by one or more of the drug delivery processes discussed elsewhere in this specification.

In the second step of the process depicted in FIG. 24, the substrate 3102/coating 3104/receptor material 3114/drug particles 3130 assembly is contacted with electromagnetic radiation to affect, e.g., the binding of the drug particles 3130 to the receptor material 3114. This may be done by, e.g., the transmission of ultrasonic radiation, as is discussed elsewhere in this specification. Alternatively, or additionally, it may be done by the use of other electromagnetic radiation that is known to affect the rate of binding between two recognition moieties and/or other biological processes.

The electromagnetic radiation may be conveyed by transmitter 3132 in the direction of arrow 3134. Alternatively, or additionally, the electromagnetic radiation may be conveyed by transmitter 3136 in the direction of arrows 3138. In the embodiment depicted in FIG. 40, both transmitter 3132 and/or transmitter 3136 are operatively connected to a controller 3140. The connection may be by direct means (such as, e.g., line 3142), and/or by indirect means (such as, e.g., telemetry link 3144).

Referring again to FIG. 24, and in the preferred embodiment depicted therein, transmitter 3132 is comprised of a sensor (not shown) that can monitor the radiation 3144 retransmitted from the surface 3114 of assembly 3103.

One may use many forms of electromagnetic radiation to affect the binding of the drug moieties 3130 to the receptor surface 3114. By way of illustration, and referring to U.S. Pat. No. 6,095,148 (the entire disclosure of which is hereby incorporated by reference into this specification), the growth and differentiation of nerve cells may be affected by electrical stimulation of such cells. As is disclosed in column 1 of such patent, “Electrical charges have been found to play a role in enhancement of neurite extension in vitro and nerve regeneration in vivo. Examples of conditions that stimulate nerve regeneration include piezoelectric materials and electrets, exogenous DC electric fields, pulsed electromagnetic fields, and direct application of current across the regenerating nerve. Neurite outgrowth has been shown to be enhanced on piezoelectric materials such as poled polyvinylidinedifluoride (PVDF) (Aebischer et al., Brain Res., 436;165 (1987); and R. F. Valentini et al., Biomaterials, 13:183 (1992)) and electrets such as poled polytetrafluoroethylene (PTFE) (R. F. Valentini et al., Brain. Res. 480:300 (1989)). This effect has been attributed to the presence of transient surface charges in the material which appear when the material is subjected to minute mechanical stresses. Electromagnetic fields also have been shown to be important in neurite extension and regeneration of transected nerve ends. R. F. Valentini et al., Brain. Res., 480:300 (1989); J. M. Kerns et al., Neuroscience 40:93 (1991); M. J. Politis et al., J. Trauma, 28:1548 (1988); and B. F. Sisken et al., Brain. Res., 485:309 (1989). Surface charge density and substrate wettability have also been shown to affect nerve regeneration. Valentini et al., Brain Res., 480:300-304 (1989).”

By way of further illustration, and again referring to U.S. Pat. No. 5,566,685, extremely low frequency electromagnetic fields may be used to cause, e.g., “. . . changes in enzyme activities . . . ,” “. . . stimulation of bone cell growth . . . ,” . . . suppression of nocturnal melatonin . . . ,” “. . . quantative changes in transcripts . . . ,” changes in “. . . gene expression of regenerating rate liver . . . ,” changes in “. . . gene expression . . . ,” changes in “. . . gene transcription . . . ,” changes in “. . . modulation of RNA synthesis and degradation . . . ,” . . . alterations in protein kinase activity . . . ,” changes in “. . . growth-related enzyme ornithine decarboxylase . . . ,” changes in embryological activity, “. . . stimulation of experimental endochondral ossification . . . ,” “. . . suppression of nocturnal melatonin . . . ,” changes in “. . . human pineal gland function . . . ,” changes in “. . . calcium binding . . . ,” etc. Reference may be had, in particular, to columns 2 and 3 of U.S. Pat. No. 5,566,685.

Referring again to FIG. 24, and to the preferred embodiment depicted therein, the transmitter 3132 preferably has a sensor to determine the extent to which radiation incident upon, e.g., surface 3146 is reflected. Information from transmitter 3132 may be conveyed to and from controller 3140 via line 3148.

In the embodiment depicted in FIG. 24, a sensor 3150 is adapted to sense the degree of binding on surface 3146 between the drug molecules 3130 and the receptor molecules 3114. This sensor 3150 preferably transmits radiation in the direction of arrow 3152 and senses reflected radiation traveling in the direction of arrow 3154. Information from and to controller 3140 is fed to and from sensor 3150 via line 3156.

There are many sensors known to those skilled in the art which can determine the extent to which two recognition molecules have bound to each other.

Thus, e.g., one may use the process and apparatus described in U.S. Pat. No. 5,376,556, in which an analyte-mediated ligand binding event is monitored; the entire disclosure of this United States patent is hereby incorporated by reference into this specification. . Claim 1 of this patent describes “A method for determining the presence or amount of an analyte, if any, in a test sample by monitoring an analyte-mediated ligand binding event in a test mixture the method comprising: forming a test mixture comprising the test sample and a particulate capture reagent, said particulate capture reagent comprising a specific binding member attached to a particulate having a surface capable of inducing surface-enhanced Raman light scattering and also having attached thereto a Raman-active label wherein said specific binding member attached to the particulate is specific for the analyte, an analyte-analog or an ancillary binding member; providing a chromatographic material having a proximal end and a distal end, wherein the distal end of said chromatographic material comprises a capture reagent immobilized in a capture situs and capable of binding to the analyte; applying the test mixture onto the proximal end of said chromatographic material; allowing the test mixture to travel from the proximal end toward the distal end by capillary action; illuminating the capture situs with a radiation sufficient to cause a detectable Raman spectrum; and monitoring differences in spectral characteristics of the detected surface-enhanced Raman scattering spectra, the differences being dependent upon the amount of analyte present in the test mixture.”

By way of further illustration, one may use the “triggered optical sensor” described and claimed in U.S. Pat. No. 6,297,059, the entire disclosure of which is hereby incorporated by reference into this specification. This patent claims (in claim 1) thereof”. An optical biosensor for detection of a multivalent target‘biomolecule comprising: a substrate having a fluid membrane thereon; recognition molecules situated at a surface of said fluid membrane, said recognition molecule capable of binding with said multivalent target biomolecule and said recognition molecule linked to a single fluorescence molecule and as being movable upon said surface of said fluid membrane; and, a means for measuring a change in fluorescent properties in response to binding between multiple recognition molecules and said multivalent target biomolecule.” In column 1 of this patent, other biological sensors are discussed, it being stated that: “Biological sensors are based on the immobilization of a recognition molecule at the surface of a transducer (a device that transforms the binding event between the target molecule and the recognition molecule into a measurable signal). In one prior approach, the transducer has been sensitive to any binding, specific or non-specific, that occurred at the transducer surface. Thus, for surface plasmon resonance or any other transduction that depended on a change in the index of refraction, such sensors have been sensitive to both specific and non-specific binding. Another prior approach has relied on a sandwich assay where, for example, the binding of an antigen by an antibody has been followed by the secondary binding of a fluorescently tagged antibody that is also in the solution along with the protein to be sensed. In this approach, any binding of the fluorescently tagged antibody will give rise to a change in the signal and, therefore, sandwich assay approaches have also been sensitive to specific as well as non-specific binding events. Thus, selectivity of many prior sensors has been a problem. Another previous approach where signal transduction and amplification have been directly coupled to the recognition event is the gated ion channel sensor as described by Cornell et al., ‘A Biosensor That Uses Ion-Channel Switches’, Nature, vol. 387, Jun. 5, 1997. In that approach an electrical signal was generated for measurement. Besides electrical signals, optical biosensors have been described in U.S. Pat. No. 5,194,393 by Hugl et al. and U.S. Pat. No. 5,711,915 by Siegmund et al. In the later patent, fluorescent dyes were used in the detection of molecules.”

By way of yet further illustration, one may use the sensor element disclosed in U.S. Pat. No. 6,589,731, the entire disclosure of which is hereby incorporated by reference into this specification. This patent, at column 1 thereof, also discusses biosensors, stating that: “Biosensors are sensors that detect chemical species with high selectivity on the basis of molecular recognition rather than the physical properties of analytes. See, e.g., Advances in Biosensors, A. P. F. Turner, Ed. JAI Press, London, (1991). Many types of biosensing devices have been developed in recent years, including enzyme electrodes, optical immunosensors, ligand-receptor amperometers, and evanescent-wave probes. The detection mechanism in such sensors can involve changes in properties such as conductivity, absorbance, luminescence, fluorescence and the like. Various sensors have relied upon a binding event directly between a target agent and a signaling agent to essentially turn off a property such as fluorescence and the like. The difficulties with present sensors often include the size of the signal event which can make actual detection of the signal difficult or affect the selectivity or make the sensor subject to false positive readings. Amplification of fluorescence quenching has been reported in conjugated polymers. For example, Swager, Accounts Chem. Res., 1998, v. 31, pp. 201-207, describes an amplified quenching in a conjugated polymer compared to a small molecule repeat unit by methylviologen of 65; Zheng et al., J. Appl. Polymer Sci., 1998, v. 70, pp. 599-603, describe a Stern-Volmer quenching constant of about 1000 for poly(2-methoxy,5-(2′-ethylhexloxy)-p-phenylene-vinylene (MEH-PPV) by fullerenes; and, Russell et al., J. Am. Chem. Soc., 1982, v. 103, pp. 3219-3220, describe a Stern-Volmer quenching constant for a small molecule (stilbene) in micelles of about 2000 by methylviologen. Despite these successes, continued improvements in amplification of fluorescence quenching have been sought. Surprisingly, a KSV of greater than 105 has now been achieved.”

Similarly, and by way of further illustration, one may use the light-based sensors discussed at column 1 of U.S. Pat. No. 6,594,011, the entire disclosure of which is hereby incorporated by reference into this specification. As is disclosed in such column 1, “It is well known that the presence or the properties of substances on a material's surface can be determined by light-based sensors. Polarization-based techniques are particularly sensitive; ellipsometry, for example, is a widely used technique for surface analysis and has successfully been employed for detecting attachment of proteins and smaller molecules to a surface. In U.S. Pat. No. 4,508,832 to Carter, et al. (1985), an ellipsometer is employed to measure antibody-antigen attachment in an immunoassay on a test surface. Recently, imaging ellipsometry has been demonstrated, using a light source to illuminate an entire surface and employing a two-dimensional array for detection, thus measuring the surface properties for each point of the entire surface in paraliel(G. Jin, R. Janson and H. Arwin, “Imaging Ellipsometry Revisited: Developments for Visualization of Thin Transparent Layers on Silicon Substrates,” Review of Scientific Instruments, 67(8), 2930-2936, 1996). Imaging methods are advantageous in contrast to methods performing multiple single-point measurements using a scanning method, because the status of each point of the surface is acquired simultaneously, whereas the scanning process takes a considerable amount of time (for example, some minutes), and creates a time lag between individual point measurements. For performing measurements where dynamic changes of the surface properties occur in different locations, a time lag between measurements makes it difficult or impossible to acquire the status of the entire surface at any given time. Reported applications of imaging ellipsometry were performed on a silicon surface, with the light employed for the measurement passing through+the surrounding medium, either air or a liquid contained in a cuvette. For applications where the optical properties of the surrounding medium can change during the measurement process, passing light through the medium is disadvantageous because it introduces a disturbance of the measurement.”

U.S. Pat. No. 6,594,011 goes on-to disclose (at columns 1-2) that: “By using an optically transparent substrate, this problem can be overcome using the principle of total internal reflection (TIR), where both the illuminating light and the reflected light pass through the substrate. In TIR, the light interacting with the substance on the surface is confined to a very thin region above the surface, the so-called evanescent field. This provides a very high contrast readout, because influences of the surrounding medium are considerably reduced. In U.S. Pat. No. 5,483,346 to Butzer, (1996) the use of polarization for detecting and analyzing substances on a transparent material's surface using TIR is described. In the system described by Butzer, however, the light undergoes multiple internal reflections before being analyzed, making it difficult or impossible to perform an imaging technique, because it cannot distinguish which of the multiple reflections caused the local polarization change detected in the respective parts of the emerging light beam. U.S. Pat. No. 5,633,724 to King, et al. (1997) describes the readout of a biochemical array using the evanescent field. This patent focuses on fluorescent assays, using the evanescent field to excite fluorescent markers attached to the substances to be detected and analyzed. The attachment of fluorescent markers or other molecular tags to the substances to be detected on the surface requires an additional step in performing the measurement, which is not required in the current invention. The patent further describes use of a resonant cavity to provide on an evanescent field for exciting analytes.”

By way of yet further illustration, one may use one or more of the biological sensors disclosed in U.S. Pat. Nos. 6,546,267 (biological sensor), 5,972,638 (biosensor), 5,854,863, 6,411,834 (biological sensor), 4,513,280 (device for detecting toxicants), 6,666,905, 5,205,292, 4,926,875, 4,947,854 (epicardial multifunctional probe), 6,523,392, 6,169,494 (biotelemetry locator), 5,284,146 (removable implanted device), 6,624,940, 6,571,125, 5,971,282, 5,766,934 (chemical and biological sensosrs having electroactive polymer thin films attached to microfabricated device and possessing immobilized indicator molecules), 6,607,480 (evaluation system for obtaining diagnostic information from the signals and data of medical sensor systems), 6,493,591, 6,445,861, 6,280,586, 5,327,225 (surface plasmon resonance sensor), and the like. The entire disclosure of each of these United States patents is hereby incorporated by reference into this specification.

In one embodiment, the biological sensor is an implantable biological sensor. One may use one or more of the implantable sensors known to those skilled in the art.)

By way of illustration, one may use the implantable extractable probe described in U.S. Pat. No. 5,205,292, the entire disclosure of which is hereby incorporated by reference into this specification. This probe comprises a biological sensor attached to the body of the probe such as, e.g., a doppler transducer for measuring blood flow.

In one embodiment, the nanowire sensor described in published U.S. patent application US20020117659 is used; the entire disclosure of this United States patent application is hereby incorporated by reference into this specification. As is disclosed in this published patent application, “The invention provides a nanowire or nanowires preferably forming part of a system constructed and arranged to determine an analyte in a sample to which the nanowire(s) is exposed. ‘Determine’, in this context, means to determine the quantity and/or presence of the analyte in the sample. Presence of the analyte can be determined by determining a change in a characteristic in the nanowire, typically an electrical characteristic or an optical characteristic. E.g. an analyte causes a detectable change in electrical conductivity of the nanowire or optical properties. In one embodiment, the nanowire includes, inherently, the ability to determine the analyte. The nanowire may be functionalized, i.e. comprising surface functional moieties, to which the analytes binds and induces a measurable property change to the nanowire. The binding events can be specific or non-specific. The functional moieties may include simple groups, selected from the groups including, but not limited to, —OH, —CHO, —COOH, —SO3H, —CN, —NH2, SH, —COSH, COOR, halide; biomolecular entities including, but not limited to, amino acids, proteins, sugars, DNA, antibodies, antigens, and enzymes; grafted polymer chains with chain length less than the diameter of the nanowire core, selected from a group of polymers including, but not limited to, polyamide, polyester, polyimide, polyacrylic; a thin coating covering the surface of the nanowire core, including, but not limited to, the following groups of materials: metals, semiconductors, and insulators, which may be a metallic element, an oxide, an sulfide, a nitride, a selenide, a polymer and a polymer gel. In another embodiment, the invention provides a nanowire and a reaction entity with which the analyte interacts, positioned in relation to the nanowire such that the analyte can be determined by determining a change in a characteristic of the nanowire.”

A drug delivery device that is comprised of a biological sensor is disclosed in published U.S. patent application US 2002/011601. As is disclosed in the “Abstract” of this published patent application, “An Implantable Medical Device (IMD) for controllably releasing a biologically-active agent such as a drug to a body is disclosed. The IMD includes a catheter having one or more ports, each of which is individually controlled by a respective pair of conductive members located in proximity to the port. According to the invention, a voltage potential difference generated across a respective pair of conductive members is used to control drug delivery via the respective port. In one embodiment of the current invention, each port includes a cap member formed of a conductive material. This cap member is electrically coupled to one of the conductive members associated with the port to form an anode. The second one of the conductive members is located in proximity to the port and serves as a cathode. When the cap member is exposed to a conductive fluid such as blood, a potential difference generated between the conductors causes current to flow from the anode to the catheter, dissolving the cap so that a biologically-active agent is released to the body. In another embodiment of the invention, each port is in proximity to a reservoir or other expandable member containing a cross-linked polymer gel of the type that expands when placed within an electrical field. Creation of an electric field between respective conductive members across the cross-linked polymer gel causes the gel to expand. In one embodiment, this expansion causes the expandable member to assume a state that blocks the exit of the drug from the respective port. Alternatively, the expansion may be utilized to assert a force on a bolus of the drug so that it is delivered via the respective port. Drug delivery is controlled by a control circuit that selectively activates one or more of the predetermined ports.”

At column 1 of published U.S. patent application US 2002/0111601, reference is made to other implantable drug delivery systems. It is disclosed that (in paragraph 0004) that “While implantable drug delivery systems are known, such systems are generally not capable of accurately controlling the dosage of drugs delivered to the patient. This is particularly essential when dealing with drugs that can be toxic in higher concentrations. One manner of controlling drug delivery involves using electro-release techniques for controlling the delivery of a biologically-active agent or drug. The delivery process can be controlled by selectively activating the electro-release system, or by adjusting the rate of release. Several systems of this nature are described in U.S. Pat. Nos. 5,876,741 and 5,651,979 which describe a system for delivering active substances into an environment using polymer gel networks. Another drug delivery system is described in U.S. Pat. No. 5,797,898 to Santini, Jr. which discusses the use of switches provided on a microchip to control the delivery of drugs. Yet another delivery device is discussed in U.S. Pat. No. 5,368,704 which describes the use of an array of valves formed on a monolithic substrate that can be selectively activated to control the flow rate of a substance through the substrate.” The disclosures of each of U.S. Pat. Nos. 5,368,704, 5,797,898, and 5,876,741 are hereby incorporated by reference into this specification.

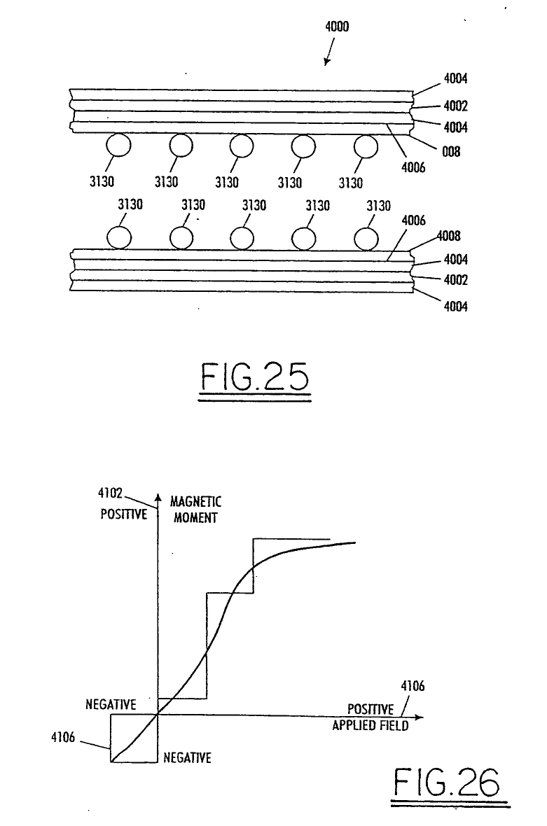

FIG. 25 is a schematic view of a preferred coated stent 4000 of the invention; as will be apparent, other coated medical devices may also be used. Referring to FIG. 25, and to the preferred embodiment depicted therein, it will be seen that coated stent 4000 is comprised of a stent 4002 onto which is deposited one or more of the nanomagnetic coatings 4004 described elsewhere in this specification. Disposed above the nanomagnetic coatings 4004 is a coating of drug-eluting polymer 4006.

One may use any of the drug eluting polymers known to those skilled in the art to produce coated stent 4000. Alternatively, or additionally, one may use one or more of the polymeric materials 14 described elsewhere in this specification.

By way of illustration, one may use the drug eluting polymeric material described in U.S. Pat. No. 5,716,981, the entire disclosure of this United States patent is hereby incorporated by reference into this specification. This patent describes and claims “A stent for expanding the lumen of a body passageway, comprising a generally tubular structure coated with a composition comprising paclitaxel, an analogue or derivative thereof, and a polymeric carrier” (see claim 1). The “polymeric carrier” may comprise poly(caprolactone), as is described in claim 2. The polymeric carrier may comprise poly (lactic) acid, as is described in claim 3. The polymeric carrier may comprise poly (ethyelne-vinyl acetate), as is described in claim 4. The polymeric carrier may comprise a copolymer of poly carprolactone and polylactic acid, as is described in claim 5.

The polymeric carrier described in U.S. Pat. No. 5,716,981 preferably is comprised of a moiety which utilize anti-angiogenic factors, i.e., factors (such as a protein, peptide, chemical, or other molecule) that acts to inhibit vascular growth. As is disclosed in this patent, “As noted above, the present invention provides compositions comprising an anti-angiogenic factor, and a polymeric carrier. Briefly, a wide variety of anti-angiogenic factors may be readily utilized within the context of the present invention. Representative examples include Anti-Invasive Factor, retinoic acid and derivatives thereof, paclitaxel, Suramin, Tissue Inhibitor of Metalloproteinase-1, Tissue Inhibitor of Metalloproteinase-2, Plasminogen Activator Inhibitor-1, Plasminogen Activator Inhibitor-2, and various forms of the lighter “d group” transition metals. These and other anti-angiogenic factors will be discussed in more detail below.”

“Briefly, Anti-Invasive Factor, or ‘AIF’ which is prepared from extracts of cartilage, contains constituents which are responsible for inhibiting the growth of new blood vessels. These constituents comprise a family of 7 low molecular weight proteins (<50,000 daltons) (Kuettner and Pauli, ‘Inhibition of neovascularization by a cartilage factor” in Development of the Vascular System, Pitman Books (CIBA Foundation Symposium 100), pp. 163-173, 1983), including a variety of proteins which have inhibitory effects against a variety of proteases (Eisentein et al, Am. J. Pathol. 81:337-346, 1975; Langer et al., Science 193:70-72, 1976: and Horton et al., Science 199:1342-1345, 1978). AWF suitable for use within the present invention may be readily prepared utilizing techniques known in the art (e.g., Eisentein et al, supra; Kuettner and Pauli, supra; and Langer et al., supra). Purified constituents of AIF such as Cartilage-Derived Inhibitor (‘CDI’) (see Moses et at., Science 248:1408-1410, 1990) may also be readily prepared and utilized within the context of the present invention.”

“Retinoic acids alter the metabolism of extracellular matrix components, resulting in the inhibition of angiogenesis. Addition of proline analogs, angiostatic steroids, or heparin may be utilized in order to synergistically increase the anti-angiogenic effect of transretinoic acid. Retinoic acid, as well as derivatives thereof which may also be utilized in the context of the present invention, may be readily obtained from commercial sources, including for example, Sigma Chemical Co. (#R2625).”

“Paclitaxel is a highly derivatized diterpenoid (Wani et al., J. Am. Chem. Soc. 93:2325, 1971) which has been obtained from the harvested and dried bark of Taxus brevifolia (Pacific Yew.) and Taxomyces Andreanae and Endophytic Fungus of the Pacific Yew (Stierle et al., Science 60:214-216, 1993). Generally, paclitaxel acts to stabilize microtubular structures by binding tubulin to form abnormal mitotic spindles. ‘Paclitaxel’ (which should be understood herein to include analogues and derivatives such as, for example, TAXOL®, TAXOTERE®, 10-desacetyl analogues of paclitaxel and 3′N-desbenzoyl-3′N-t-butoxy carbonyl analogues of paclitaxel) may be readily prepared utilizing techniques known to those skilled in the art (see also WO 94/07882, WO 94/07881, WO 94/07880, WO 94/07876, WO 93/23555, WO 93/10076, U.S. Pat. Nos. 5,294,637, 5,283,253, 5,279,949, 5,274,137, 5,202,448, 5,200,534, 5,229,529, and EP 590267), or obtained from a variety of commercial sources, including for example, Sigma Chemical Co., St. Louis, Miss. (T7402-from Taxus brevifolia).”

“Suramin is a polysulfonated naphthylurea compound that is typically used as a trypanocidal agent. Briefly, Suramin blocks the specific cell surface binding of various growth factors such as platelet derived growth factor (‘PDGF’), epidermal growth factor (‘EGF’), transforming growth factor (‘TGF-β’), insulin-like growth factor (‘IGF-I’), and fibroblast growth factor (‘βFGF’). Suramin may be prepared in accordance with known techniques, or readily obtained from a variety of commercial sources, including for example Mobay Chemical Co., New York. (see Gagliardi et al., Cancer Res. 52:5073-5075, 1992; and Coffey, Jr., et al., J. of Cell. Phys. 132:143-148, 1987).”

“A wide variety of other anti-angiogenic factors may also be utilized within the context of the present invention. Representative examples include Platelet Factor 4 (Sigma Chemical Co., #F1385); Protamine Sulphate (Clupeine) (Sigma Chemical Co., #P4505); Sulphated Chitin Derivatives (prepared from queen crab shells), (Sigma Chemical Co., #C3641; Murata et al., Cancer Res. 51:22-26, 1991); Sulphated Polysaccharide Peptidoglycan Complex (SP-PG) (the function of this compound may be enhanced by the presence of steroids such as estrogen, and tamoxifen citrate); Staurosporine (Sigma Chemical Co., #S4400); Modulators of Matrix Metabolism, including for example, proline analogs {[(L-azetidine-2-carboxylic acid (LACA) (Sigma Chemical Co., #A0760)), cishydroxyprohine, d,L-3,4-dehydroproline (Sigma Chemical Co., #D0265), Thiaproline (Sigma Chemical Co., #T063 1)], .alpha.,.alpha.-dipyridyl (Sigma Chemical Co., #D7505), β-aminopropionitrile fumarate (Sigma Chemical Co., #A3134)]}; MDL 27032 (4-propyl-5-(4-pyridinyl)-2(3H)-oxazolone; Merion Merrel Dow Research Institute); Methotrexate (Sigma Chemical Co., #A6770; Hirata et al., Arthritis and Rheumatism 32:1065-1073, 1989); Mitoxantrone (Polverini and Novak, Biochem. Biophys. Res. Comm. 140:901-907); Heparin (Folkman, Bio. Phar. 34:905-909, 1985; Sigma Chemical Co., #P8754); Interferons (e.g., Sigma Chemical Co., #13265); 2 Macroglobulin-serum (Sigma Chemical Co., #M7151); ChIMP-3 (Pavloff et al., J. Bio. Chem. 267:17321-17326, 1992); Chymostatin (Sigma Chemical Co., #C7268; Tomkinson et al., Biochem J. 286:475-480, 1992); β-Cyclodextrin Tetradecasulfate (Sigma Chemical Co., #C4767); Eponemycin; Camptothecin; Fumagillin (Sigma Chemical Co., #F6771; Canadian Patent No. 2,024,306; Ingber et al., Nature 348:555-557, 1990); Gold Sodium Thiomalate (“GST”; Sigma:G4022; Matsubara and Ziff, J. Clin. Invest. 79:1440-1446, 1987); (D-Penicillamine (“CDPT”; Sigma Chemical Co., #P4875 or P5000(HCl)); β-1-anticollagenase-serum; .alpha.2-antiplasmin (Sigma Chem. Co.:A0914; Holmes et al., J. Biol. Chem. 262(4):1659-1664, 1987); Bisantrene (National Cancer Institute); Lobenzarit disodium (N-(2)-carboxyphenyl-4-chloroanthronilic acid disodium or “CCA”; Takeuchi et al., Agents Actions 36:312-316, 1992); Thalidomide; Angostatic steroid; AGM-1470; carboxynaminolmidazole; metalloproteinase inhibitors such as BB94 . . . . ”

The polymeric carrier may be, e.g., a polyvinyl aromatic polymer, as is disclosed in U.S. Pat. No. 6,306,166, the entire disclsoure of which is hereby incorporated by reference into this specification. As is disclosed in this patent, some suitable polyvinyl aromatic polymers include a polymter that is “. . . hydrophilic or hydrophobic, and is selected from the group consisting of polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate and blends and copolymers thereof as well as other biodegradable, bioabsorbable and biostable polymers and copolymers. Coatings from polymer dispersions such as polyurethane dispersions . . . and acrylic latex dispersions are also within the scope of the present invention. The polymer may be a protein polymer, fibrin, collage and derivatives thereof, polysaccharides such as celluloses, starches, dextrans, alginates and derivatives of these polysaccharides, an extracellular matrix component, hyaluronic acid, or another biologic agent or a suitable mixture of any of these, for example. In one embodiment of the invention, the preferred polymer is polyacrylic acid, available as HYDROPLUS® (Boston Scientific Corporation, Natick, Mass.), and described in U.S. Pat. No. 5,091,205, the disclosure of which is hereby incorporated herein by reference. U.S. Pat. No. 5,091,205 describes medical devices coated with one or more polyisocyanates such that the devices become instantly lubricious when exposed to body fluids. In a most preferred embodiment of the invention, the polymer is a copolymer of polylactic acid and polycaprolactone.”

In one embodiment, the polymeric carrier is a water souble polymer, such as the water soluble polymers disclose in U.S. Pat. No. 6,441,025, the entire dislcosure of which is hereby incorporated by reference into this specification. These polymers include, e.g., “. . . a water soluble-polymer having a molecular weight of at least about 5,000 D and dispersed in a pharmaceutically acceptable solution . . . . ” (claim 1), “. . . poly-glutamic acids, poly-aspartic acids or poly-lysines . . . ” (claim 13), etc.

In one embodiment, the polymeric carrier is a biocompatible, pharmaceutically active, bioerodible polymer, as that term is used and defined in published U.S. patent application US 2002/0042645. The entire disclosure of this published U.S. patent application is hereby incorporated by reference into this specificaiton. As is disclosed in this published patent application: “This invention generally embraces drug eluting stented grafts wherein the drug eluting capability is provided by a composite of drug material and a bioerodible polymer. A feature of the invention is the discovery of a particularly useful group of bioerodible polymers for this purpose. These polymers are fully described In U.S. Pat. No. 4,131,648 by Nam S. Choi and Jorge Heller, issued Dec. 26, 1978, assigned to Alza Corporation, and entitled “Structured Orthoester and Orthocarbonate Drug Delivery Devices”, which is incorporated herein in its entirety by reference. The patent discloses a class of polymers comprising a polymeric backbone having a repeating unit comprising hydrocarbon radicals and a symmetrical dioxycarbon unit with a multiplicity of organic groups bonded thereto. The polymers prepared by the invention have a controlled degree of hydrophobicity with a corresponding controlled degree of erosion in an aqueous or like environment to innocuous products. The polymers can be fabricated into coatings for releasing a beneficial agent, as the polymers erode at a controlled rate, and thus can be used as carriers for drugs for releasing drug at a controlled rate to a drug receptor, especially where bioerosion is desired.”

Some of the polymers specifically described in the claims of published U.S. patent application US 2002/0042645 include, e.g., “. . . a biocompatible, pharmaceutically acceptable, bioerodible polymer . . . . ,” “. . . a polyester . . . ,” “. . . a hydrophobic, bioerodible, copolymer comprising mers I and II according to the following formula: . . . . ” (see claim 6), a polymer in which “. . . a multiplicity of microcapsules is dispersed within said at least one polymer, wherein said microcapsules have a wall formed of a drug release rate controlling material; said at least one therapeutic substance is contained within said multiplicity of microcapsules . . . ,” “. . . a pharmaceutically acceptable biocompatible non-bioerodible polymer that sequesters an agent for brachytherapy . . . ,”

Referring again to FIG. 25, and to the preferred embodiment depicted therein, disposed on the surface 4008 of the drug eluting polymer are a multiplicity of magnetic drug particles, such the magnetic drug particle 3130 (see FIG. 22).

FIG. 26 is a graph of a typical response of a magnetic drug particle, such as magnetic drug particles 3130 (see, e.g., FIG. 22) to an applied electromagnetic field. As will be seen by reference to FIG. 26, as the magnetic field strength 4100 of an applied mangetic field is increased along the positive axis, the magnetic moment 4102 of the magnetic drug particle(s) also continuously increases along the positive axis. As will be apparent, a decrease in the magnetic field strength also causes a decrease in magnetic moment. Thus, when the polarity of the applied magnetic field changes (see section 4106 of the graph), the magnetic moment also decreases. Thus, one may affect the magnetic moment of the magnetic drug particles by varying either the intensity of the applied electromagnetic field and/or its polarity.

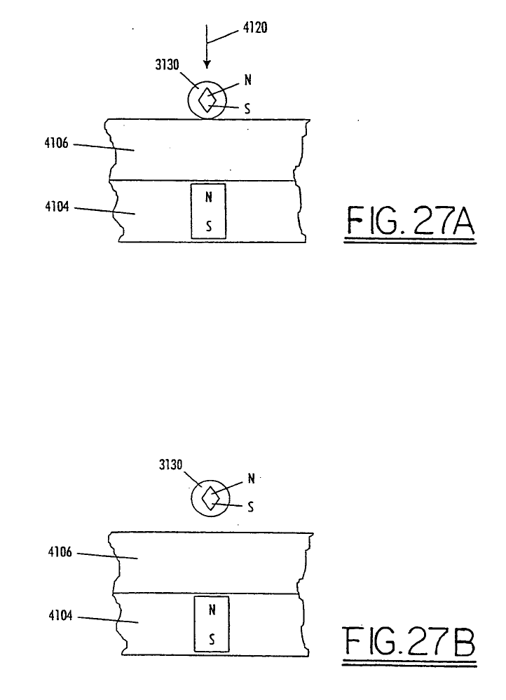

FIGS. 27A and 27B illustrate the effect of applied fields upon the nanomagnetic coating 4004 (see FIG. 25) and the magnetic drug particles 3130. Referring to FIG. 27A, when the applied magnetic field 4120 is sufficient to align the drug particle 3130 in a north(up)/south(down) orientation (see FIG. 27A), it will also tend to align the nanomagnetic material is such an orientation. However, because the magnetic hardness of the nanomagentic material will be chosen to substantially exceed the magnetic hardness of the drug particles 3130, then the applied magnetic field will not be able to realign the nanomagnetic material.

In the ensuing discussion relating to the effects of an applied electromagnetic field, certain terms (such as, e.g., “magnetization saturation”) will be used. These terms (and others) have the meaning set forth in several of applicants' published patent applications and patents, including (without limitation) published patent application US20030107463, U.S. Pat. Nos. 6,700,472, 6,673,999, 6,506,972, 5,540,959, and the like. The entire disclosure of each of these documents is hereby incorporated by reference into this specification.

Thus, by way of illustration, reference is made to the term “magnetization.” As is disclosed in applicants' publications, magnetization is the magnetic moment per unit volume of a substance. Reference may be had, e.g., to U.S. Pat. Nos. 4,169,998, 4,168,481, 4,166,263, 5,260,132, 4,778,714, and the like. The entire disclosure of each of these United States patents is hereby incorporated by reference into this specification.

Thus, by way of further illustration, reference is made to the term “saturation magnetization.” As is disclosed in applicants' publications, for a discussion of the saturation magnetization of various materials, reference may be had, e.g., to U.S. Pat. Nos. 4,705,613, 4,631,613, 5,543,070, 3,901,741 (cobalt, samarium, and gadolinium alloys), and the like. The entire disclosure of each of these United States patents is hereby incorporated by reference into this specification. As will be apparent to those skilled in the art, especially upon studying the aforementioned patents, the saturation magnetization of thin films is often higher than the saturation magnetization of bulk objects.

By way of further illustration, reference is made to the term “coercive force.” As is disclosed in applicants' publications, the term coercive force refers to the magnetic field, H, which must be applied to a magnetic material, in a symmetrical, cyclicly magnetized fashion, to make the magnetic induction, B, vanish; this term often is referred to as magnetic coercive force. Reference may be had, e.g., to U.S. Pat. Nos. 4,061,824, 6,257,512, 5,967,223, 4,939,610, 4,741,953, and the like. The entire disclosure of each of these United States patents is hereby incorporated by reference into this specification.

In one embodiment, the nanomagnetic material 103 has a coercive force of from about 0.01 to about 3,000 Oersteds. In yet another embodiment, the nanomagnetic material 103 has a coercive force of from about 0.1 to about 10.

By way of yet further illustration, reference is made to the term relative magnetic permeability. As is disclosed in applicants' publications, the term relative magnetic permeability is equal to B/H, and is also equal to the slope of a section of the magnetization curve of the film. Reference may be had, e.g., to page 4-28 of E. U. Condon et al.'s “Handbook of Physics” (McGraw-Hill Book Company, Inc., New York, 1958). Reference also may be had to page 1399 of Sybil P. Parker's “McGraw-Hill Dictionrary of Scientific and Technical Terms,” Fourth Edition (McGraw Hill Book Company, New York, 1989). As is disclosed on this page 1399, permeability is “. . . a factor, characteristic of a material, that is proportional to the magnetic induction produced in a material divided by the magnetic field strength; it is a tensor when these quantities are not parallel. Reference also may be had, e.g., to U.S. Pat. Nos. 6,181,232, 5,581,224, 5,506,559, 4,246,586, 6,390,443, and the like. The entire disclosure of each of these United States patents is hereby incorporated by reference into this specification.

Referring again to FIG. 27, and in the preferred embodiment depicted therein, the magnetic hardness of the nanomagnetic material 4104 is preferably at least about 10 times as great as the magnetic hardness of the drug particles 3130. The term “magnetic hardness” is well known to those skilled in the art. Reference may be had, e.g., to the claims and specifications of U.S. Pat. Nos. 6,201,390, 5,595,454, 5,451,162, 6,534,984, 4,967,078, 3,802,854, and the like. The entire disclosure of each of these United States patents is hereby incorporated by reference into this specification.

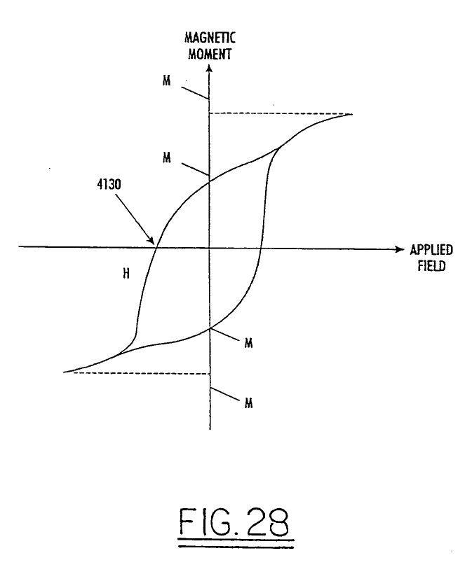

FIG. 28 is graph of a preferred nanomagnetic material and its response to an applied electromagnetic field, in which the applied field is applied against the magnetic moment of the nanomagnetic material.

As will be apparent from this FIG. 28, a certain amount of the applied electromagnetic force is required to overcome the remnant magnetization (Mr) and to change the direction of the remant magnetization from +Mr to −Mr. Thus, e.g., the point −Hc, at point 4130, indicates how much of the field is required to make the magnetic moment be zero.

Referring again to FIGS. 27A and 27B, and in the preferred embodiments depicted therein, the Hc values of the nanomagnetic material chosen will be sufficient to realign to magnetic drug particles 3130 but insufficient to realign the nanomagnetic material. The resulting situation is depcited in FIGS. 27A and 27B.

In FIG. 27A, with the appropriate applied magnetic field, the magnetic drug particle 3130 is attached to the nanomagnetic material 4104 and thus will tend to diffuse into the polymer 4106. By comparison, in the situation depicted in FIG. 27B, the mangetic drug partigcles will be repelled by the nanomagnetic materail. Thus, and as will be apprent, by the appropriate choice of the applied magneticfield, one can cause the magnetic drug particles either to be attracted to the layer of poolymer mateiral 4106 or to be repelled therefrom.

FIG. 29 illustrates the forces acting upon a magnetic drug particle 3130 as it approaches the nanomagnetic material 4104. Referring to FIG. 29, and in the preferred embodiment depicted therein, a certain hydrodynamic force 4140 will be applied to the particle 3130 due to the force of flow of bodily fluid, such as blood. Simultaneously, a certain attractive force 4142 will be created by the attraction of the nanomagnetic material 4104 and the particle 3130. The resulting force vector 4144 will tend to be the direction the particle 3130 will travel in. If the surface of the polymeric material is preferably comprised of a multplicity of pores 4146, the entry of the drug particles 3130 will be facilitated into such pores.

FIG. 30 illustrates the situation that occurs after the drug particles 3130 have migrated into the layer of polymeric material and when one desires to release such drug particles. In this situation (see FIG. 27B), the applied magnetic field will be chosen such that the nanomagnetic material will tend to repel the drug particles 3130 and cause their departure into bodily fluid in the direction of arrow 4148.

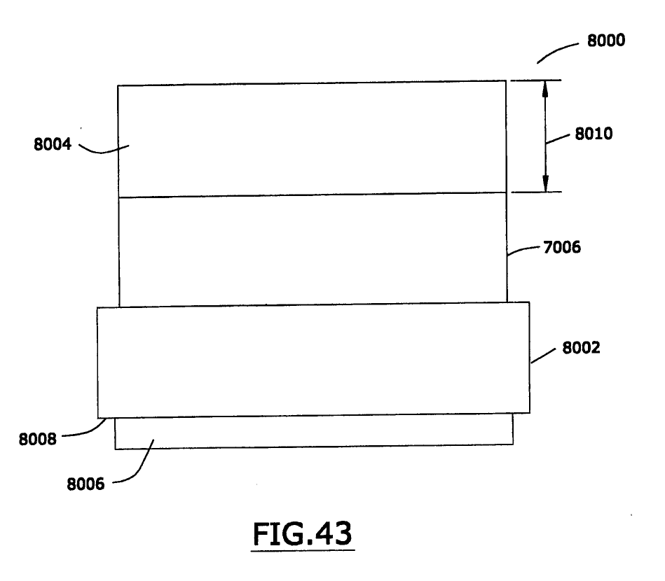

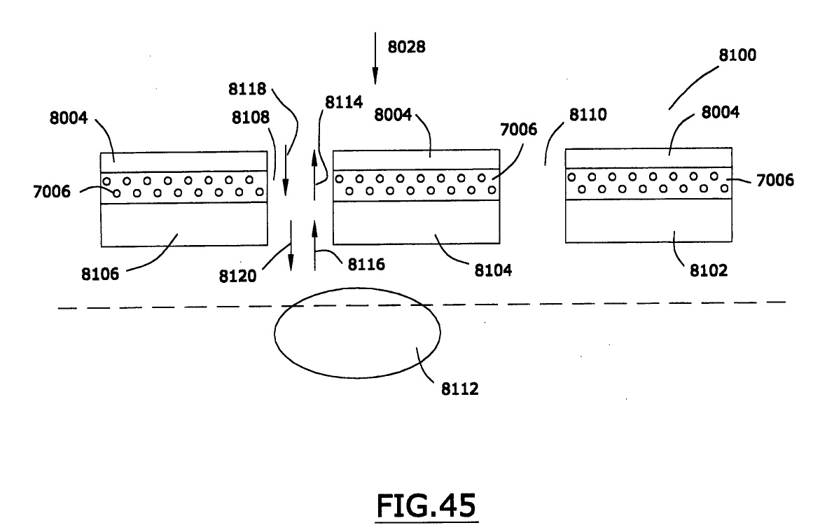

FIG. 31 illustrates the situation that occurs after the drug particles 3130 have migrated into the layer of polymeric material 4106 but when no external electromagnetic field is imposed. In this situation, there will still be an attraction between the nanomagnetric material 4104 and the magnetric drug particles 3130 that will be sufficient to keep such particles bound. However, the attraction will be weak enough such that, when hydrodynamic force 4140 is applied (see FIG. 45), the particles 3130 will elute into the bodily fluid (not shown). As will be apparent, the degree of elution in this case is less than the degree of elution in the case depicted in FIG. 43B. Thus, by the apprpropriate choice of electromagnetic field 4120, one can control the rate of depositoin of the drug particles 3130 onto the polymer 4106, or from ‘the polymer 4106.

Magnetic Drug Compositions

In this section of the specification, applicants will describe certain magnetic drug compositions 3130 that may be used in their preferred process. Each of these drug compositions preferably is comprised of at least one therapeutic agent and has a magnetic moment so that it can be attracted to or repelled from the nanomagnetic coatings upon application of an external electromagnetic field.

One such magnetic composition is disclosed in U.S. Pat. No. 2,971,916, the entire disclosure of which is hereby incorporated by reference into this specification. This patent discloses and claims a microscopic capsule having a wall of hardened organic colloid material enclosing a dispersion of magnetic powder. In one embodiment, the magnetic powder is comprised of the nanomagnetic particles of this invention.

Another such magnetic composition is disclosed in U.S. Pat. No. 3,663,687, the entire disclosure of which is hereby incorporated by reference into this specification. This patet discloses tiny, substantiallyspherular particles comprised of a parenterally metabolizable protein (such as albumin) and which are labeled with a radioisotope. At column 1 of this patent, it is disclosed that: “It has heretofore been known to encapsulate natural products for food or pharmaceutical use in proteinaceous materials such as gelatin and albumin, and small spherical particles of such encapsulated materials have been made, e.g., by processes such as those disclosed in U.S. Pat. Nos. 3,137,631; 3016,308; 3,202,731; 2,800,457, and the like.” The entire disclosure of each of these patents is hereby incorporated by reference into this specification.

Another such magnetic drug composition is disclosed in U.S. Pat. No. 4,101,435, the entire disclosure of which is hereby incorporated by reference into this specification. This patent claims “A water dispersable magnetic iron oxide-dextran complex wherein the proportion of the dextran . . . is about 0.1 to about 1 mole per mole of iron oxide . . . . ” This complex is a “magnetic iron oxide sol” is stable and non-toxic. In one embodiment, the magnetic iron oxide material of this patent is replaced by the nanomagnetic material of this invention.

Another such magnetic drug composition is disclosed in U.S. Pat. No. 4,230,685, the entire disclosure of which is hereby incorporated by reference into this specification. This patent discloses “magnetically-responsive microspheres” prepared from a mixture of albumin, magnetic particles (e.g., magnetite), and a protein bound to the outer surfaces of the microspheres. In column 5 of the patent, attachment of specific antibodies (such as staphylococcal Protein A) to the microspheres is discussed. The magnetite of this patent may advantageously be replaced by the nanomagnetic material of this invention.

A similar magnetic drug composition is disclosed in U.S. Pat. No. 4,247,406, the entire disclosure of which is hereby incorporated by reference into this specification. This patent claims (see claim 1) “An intravascularly-administrable, magnetically localizable biodegradable carrier, comprising microspheres formed from an amino acid polmer matrix with magnetic particles embedded therein . . . . ” Example 1 of this patent disclosed the preparation of a microcapsule comprised of 21 percent of magnetite, 73 percent of albumin, and 5 percent of adriamycin. The magnetic particles used in the process of U.S. Pat. No. 4,247,406 may advantageously be replaced by the nanomagnetic particles of this invention.

U.S. Pat. No. 4,247,406 discloses an intravascularly-administrable, magnetically localizable biodegradable carrier that is comprised of microspheres formed from an aminoacid polymer matrix with magnetic particles embedded therein. At column 4 of the patent, it is disclosed that “The carrier of this invention is believed to be of particular value for administering water-soluble chemotherapeutic agents, such as anti-cancer agents . . . . ” In Example 2 of the patent, the preparation of a microsphere containing 50 percent of magnetite, 46 percent of albumin, and 4 percent of adriamycin is disclosed. The magnetite particles of this patent may advantageously be replaced by the nanomagnetic particles of this invention.

U.S. Pat. No. 4,331,654 discloses and claims: “A magnetically-localizable, biodegradable, substantially water-free drug carrier formulation consisting essentially of lipid microspheres containing a magnetically-responsive substance, one or more biodegradable lipids, and one or more non-toxic surfactants.” The entire disclosure of this United States patent is hereby incorporated by reference into this specification. The magnetically-responsive substance of this patent may be replaced by the nanomagnetic particles of this invention.

At columns 1-3 of U.S. Pat. No. 4,331,654, a substantial amount of prior art is disclosed regarding magnetically-localizable biodegradable albumin microspheres. Thus, e.g., it is disclosed that: “Magnetically-localizable, biodegradable albumen microspheres have been described by Widder et al., Proc. Soc. Exp. Biol. Med., 58, 141 (1978). The use of such microspheres containing the anticancer drug, adriamycin, in treating rats bearing a Yoshida sarcoma is described in an abstract of a paper by Widder et al., given at the annual meeting of the American Association for Cancer’ Research in May of 1980 and also at the Federated Societies Meeting in San Francisco, April 1980. Magnetically-localizable, biodegradable albumen microspheres are also described and claimed in the copending application of Senyei and Widder, Ser. No. 32,399 filed Apr. 23, 1979, now U.S. Pat. No. 4,247,406.”

U.S. Pat. No. 4,331,654 also discloses that “U.S. Pat. No. 4,115,534 discloses a method for determining the concentration of various substances in biological fluids by using magnetically-responsive, permeable, solid, water-insoluble microparticles. The water-insoluble permeable solid matrix can be composed of proteinaceous materials, polysaccharides, polyurethanes or mixtures thereof. The magnetically-responsive material employed is BaFe12 O19. This material is mixed with, for example, bovine serum albumen and the resulting mixture added to a solution comprising a dewatering agent, a cross-linking agent and castor oil. A dispersion of the aqueous material in the oil phase is produced thereby. Particles thus formed are employed in vitro for determining concentrations of various substances in biological fluids.” The water-insoluble microparticles of this patent may be replaced by the nanomagnetic particles of this invention.

U.S. Pat. No. 4,331,654 also discloses that “An abstract of a Japanese patent, Chemical Abstracts, 80, 52392a (1974), describes a magnetic material coated with an organic polymer. The combination can be used as a carrier for drugs and x-ray contrast media. For instance, if the material is given orally to an ulcer patient, the magnet localizes the iron-bearing polymer of the lesion and sharp x-ray photos are obtained. Another Japanese advance has been described in the recent press wherein microspheres of a biodegradable nature containing a drug were coated with magnetic particles and the coated microspheres are injected into an animal. The microspheres thus prepared were in excess of 10 microns in diameter.”

U.S. Pat. No. 4,331,654 also discloses that “Figge et al, U.S. Pat. No. 3,474,777, disclose and claim finely divided particles of a magnetically-responsive substance having a coating of a therapeutic agent thereon, said particles being injectable.

No actual examples are given. Schleicher et al, U.S. Pat. No. 2,971,916, describe the preparation of pressure-rupturable microscopic capsules having contained therein, in suspension in a liquid vehicle, micro-fine particles of a magnetic material useful in printing. U.S. Pat. No. 2,671,451 discloses and claims a remedial pill containing a substance soluble in the human body and including a magnetically-attractable metal element. No specific materials are disclosed. U.S. Pat. No. 3,159,545 discloses a capsule formed of a non-toxic, water-soluble thermoplastic material and a radioactive composition compounded from pharmaceutical oils and waxes in the said capsule. The capsule material is usually gelatin. U.S. Pat. No. 3,190,837 relates to a minicapsule in which the core is surrounded first by a film of a hydrophylic film-forming colloid (first disclosed in U.S. Pat. No. 2,800,457) and a second and different hydrophylic film-forming colloid adherantly surrounding the core plus the first hydrophylic film. Successive deposits of capsule or wall material may also be employed. Among the core materials are mentioned a number of magnetic materials including magnetic iron oxide. A large number of oils may also be employed as core materials but these are, as far as can be seen, not pharmacologically active. Finally U.S. Pat. No. 3,042,616 relates to a process of preparing magnetic ink as an oil-in-water emulsion.”

U.S. Pat. No. 4,331,654 also discloses that “There are a number of references which employ lipid materials to encapsulate various natural products. For example, U.S. Pat. No. 3,137,631 discloses a liquid phase process for encapsulating a water-insoluble organic liquid, particularly an oil or fragrance, with albumen. The albumen coating is then denatured, and the whole aerated. Specific examples include the encapsulation of methyl benzoate, pinene or bornyl acetate and the like in egg albumen. U.S. Pat. No. 3,937,668 discloses a similar product useful for carrying radioactive drugs, insecticides, dyes, etc. Only the process of preparing the microspheres is claimed. U.S. Pat. No. 4,147,767 discloses solid serum albumen spherules having from 5 to 30% of an organic medicament homogenously entrapped therein. The spherules are to be administered intravascularly. Zolle, the patentee of U.S. Pat. No. 3,937,668 has also written a definitive article appearing in Int. J. Appl. Radiation Isotopes, 21, 155 (1970). The microspheres disclosed therein are too large to pass into capillaries and are ultimately abstracted from the circulation by the capillary bed of the lungs. U.S. Pat. No. 3,725,113 discloses microencapsulated detoxicants useful on the other side of a semipermeable membrane in a kidney machine. In this application of the microencapsulation art, the solid detoxicant is first coated with a semipermeable polymer membrane and secondly with a permeable outer layer consisting of a blood-compatible protein. U.S. Pat. No. 3,057,344 discloses a capsule to be inserted into the digestive tract having valve means for communicating between the interior of the capsule and exterior, said valve being actuable by a magnet. Finally, German Offenlegungsschrift, No. P. 265631 7.7 filed Dec. 11, 1976 discloses a process wherein cells are suspended in a physiological solution containing also ferrite particles. An electric field is applied thereto thereby causing hemolysis. A drug such as methotrexate is added as well as a suspension of ferrite particles. The temperature of the suspension is then raised in order to heal the hemolysed cells. The final product is a group of cells loaded with ferrite particles and containing also a drug, which cells can be directed to a target in vivo by means of a magnet.”

U.S. Pat. No. 4,331,654 also discloses that “Lipid materials, particularly liposomes have also been employed to encapsulate drugs with the object of providing an improved therapeutic response. For example, Rahman et al, Proc. Soc. Exp. Biol. Med., 146, 1173 (1974) encapsulated actinomycin D in liposomes. It was found that actinomycin D was less toxic to mice in the liposome form than in the non-encapsulated form. The mean survival times for mice treated with actinomycin D in this form were increased for Ehrlich ascites tumor. Juliano and Stamp, Biochemical and Biophysical Research Communications, 63, 651 (1975) studied the rate of clearance of colchicine from the blood when encapsulated in a liposome and when non-encapsulated.”

U.S. Pat. No. 4,331,654 also discloses that “Among the major contributors to this area of research—use of liposomes—has been Gregoriades and his co-workers. Their first paper concerned the rate of disapparence of protein-containing liposomes injected into a rate [Brit. J. Biochem., 24, 485 (1972)]. This study was continued in Eur. J. Biochem., 47, 179 (1974) where the rate of hepatic uptake and catabolism of the liposome-entrapped proteins was studied. The authors believed that therapeutic enzymes could be transported via liposomes into the lysosomes of patients suffering from various lysosomal diseases. In Biomedical and Biophysical Research Communications 65, 537 (1975), the group studied the possibility of holding liposomes to target cells using liposomes containing an antitumor drug. The actual transport of an enzyme, horseradish peroxidase, to the liver via liposomes was discussed in an abstract for 7th International Congress of the Reticuloendothelial Society, presented at Pamplona, Spain, Sep. 15-20, 1975.”

By way of further illustration, U.S. Pat. No. 4,345,588 dislcoses a method of delivering a water-soluble anti-cancer agent to a target capillary bed of a body associated with a tumor, comprising the step of incorporating the water-soluble anti-cancer agent into microspheres formed from a biodegradable matrix material, and thereafter applying a magnetic field to immobilize the microspheres. Claim 4 of this patent, which is typical, describes: “The method of delivering a water soluble anti-cancer agent to a target capillary bed of the body associated with a tumor, comprising the steps of: (a) incorporating the water-soluble anti-cancer agent in microspheres formed from a biodegradable matrix material with magnetic particles embedded therein, said magnetic particles having an average size of not over 300 Angstroms, said microspheres having an average size of less than 1.5 microns and passing into said capillary bed with the blood flowing therethrough, said microspheres containing from 10 to 150 parts by weight of said magnetic particles per 100 parts of said matrix material; (b) introducing said anit-cancer agent containing microspheres into an artery upstream of said capillary bed; (c) applying a magnetic field to the area of the body of said capillary bed and artery, said magnetic field being of a strength capable of immobilizing said microspheres at the blood flow rate of said capillary bed while permitting said microspheres to pass through said artery at the blood flow rate therein; (d) immobilizing at least part of said microspheres in capillaries of said target bed by said magnetic field application while blood continues to perfuse therethrough; and (e) removing said magnetic field before said anti-cancer agent is released from said microspheres, said microspheres being retained in said capillary bed after said removal of said magnetic field for release of said anti-cancer agent in effective therapeutic relation to said tumor.” The operation of this claimed invention is described in part at column 2 of the patent, wherein it is disclosed that: “The present invention provides a novel method of delivering a therapeutic agent to a target capillary bed of the body. The method takes advantage of the difference in blood flow rates between arteries and capillaries. The magnetic microspheres used for administering the therapeutic agent are selectively localized in the target capillary bed by applying a magnetic field which immobilizes the microspheres at the much slower blood flow rate of the capillaries but not at the flow rate of the arteries into which the microspheres are initially introduced. Moveover, the magnetic field need be applied only for a short time, after which it can be removed. This is based on the discovery that microspheres of sufficiently small size can be permanently localized in the capillaries, once they have been magnetically attracted to the walls of the capillaries and immobilized thereon, even though the blood continues to flow through the capillary bed in a substantially normal manner. In other words, the immobilized microspheres do not plug-up or block the capillaries as described in the method of U.S. Pat. No. 3,663,687 . . . . For effective magnetic control, the microspheres are introduced into an artery upstream of the capillary bed where they are to be localized, the selected capillary bed being associated with the target site. It is therefore of critical importance that the microspheres have a degree of magnetic responsiveness which permit them to pass through the arteries without significant holdup under the applied magnetic field while being immobilized and retained in the capillaries. The present invention achieves this objective by utilizing the difference in flow rates of the blood in the larger arteries and in the capillaries. In addition, the albumin surface prevents clump formation, thus allowing relatively normal blood perfusion at the area of retention.”

One may use the process of this patent with the nanomagnetic particles of this invention in substantial accordance with the procedure of such patent. Once the nanomagnetic particles have been delivered to the desired site, another electromagnetic field may be applied to cause such particles to heat up to a certain specified temperature at which one or,more therapeutic objectives may be attained. Once the temperature of the naoparticles exceeds the desired temperature, the heating of such particles ceases (see FIG. 4C).

U.S. Pat. No. 4,357,259 discloses a process for incorporating water-soluble therapeutic agents into albumin microspheres. Among the agents that may be so incorporated are included enzymes (such as, e.g., trypsinogen, chymotrypsinogen, plasminogen, streptokinase, adenyl cyclase, insulin, glucagons, coumarin, heparin, histamine, and the like), chemotherapeutic agents (such as, e.g., tetracycline, aminoglycosides, penicillin group of drugs, +Cephalosporins, sulfonamide drugs, chloramphenicol sodium succinate, erythromycin, vancomycin, lincomycin, clindamycin, nystatin, amphotericin B, amantidine, idoxuridine, p-Amino salicyclic acid, isoniazid, rifampin, water-soluble alkylating agents in Ca therapy, water-soluble antimetabolites, antinomycin D, mithramycin, daunomycin, adriamycin, bleomycin, vinblastine, vincristine, L-asparaginase, procarbazine, imidazole carboxamide, and the like), immunological adjuvans (such as, e.g., concanavalin A, BCG, levamisole, and the like), natural products (such as, e.g., prostaglandins, PGE1, PGE2, cyclic nucleotides, TAF antagonists, water-soluble hormones, lymphocyte inhibitors, lymphocyte stimulatory products, and the like), etc. In addition to such therapeutic agents, one may also incorporate the nanomagnetic particles of this invention into such microspheres.

Claim 1 of U.S. Pat. No. 4,357,259 is typical of the process of the patent. Such claim 1 describes: “The method of incorporating a water-soluble heat-sensitive therapeutic agent in albumin microspheres, in which all steps thereof are carried out at a temperature within the range from 1° to 45° C., said method including the steps of preparing an aqueous albumin solution of the said therapeutic agent, said albumin solution containing from 5 to 50 parts by weight of albumin per 100 parts of water and from 1 to 20 parts by weight of said therapeutic agent per 100 parts of albumin, emulsifying said albumin solution with a vegetable oil to form a water-in-oil emulsion containing dispersed droplets of the albumin solution, removing the oil by washing the dispersed droplets with an oil-soluble water-immiscible organic solvent, and recovering the resulting microspheres, wherein said method also includes the step of contacting said microspheres with an organic solvent solution of an aldehyde hardening agent to increase the stability of said microspheres and to decrease the release rate of said drug therefrom.” Claim 3 of the patent, which is dependent upon claim 1, further recites that “. . . the albumin solution also contains magnetic particles.” The “magnetic particles” of such claim 3 may be applicants' nanomagnetic particles.

U.S. Pat. No. 4,501,726 discloses a magnetically responsive nanoparticle made up of a crystalline carbohydrate matrix. Claim 1 of this patent, which is typical, describes: “A nanosphere or nanoparticle for intravascular administration, which is magnetically responsive and biologically degradable and which is made up of a matrix in which a magnetic material is enclosed, characterized in that said nanosphere or nanoparticle has an average diameter which does not exceed 1500 nm, and circulates in the vascular system after administration thereto, said matrix comprising a hydrophillic, crystalline carbohydrate.”

The carbohydrate matrix of the particle of U.S. Pat. No. 4,501,726 is biodegradable. Furthermore, one or more drugs may be adsorbed to the carbohydrate after the nanoparticles have been produced. As is disclosed in column 2 of U.S. Pat. No. 4,501,726, “Carbohydrate polymers containing alpha(1-4) bonds are especially useful because they can be degraded by the alpha-amylase in the body. Although starch is preferred, also pullullan, glycogen and dextran may be used. It is also possible to modify the carbohydrate polymer with, for example, hydroxyethyl, hydroxypropyl, acetyl, propionyl, hydroxypropanoyl, various derivatives of acrylic acid or like substituents. Also carbohydrates which are not polymeric, may be used in the context of this invention. Examples of such carbohydrates are glucose, maltose and lactose. Pharmaceuticals may be adsorbed to the carbohydrates after the nanosphere has been produced. This may be important in such cases where the pharmaceutical in question is damaged by the treatment in connection with the production of the magnetic nanospheres. If the matrix is a carbohydrate, it is also possible to modify the matrix by covalently coupling to the carbohydrate e.g. amino groups or carboxylic acid groups, thereby to create an adsorption matrix. High molecular substances of the type proteins may be enclosed within the matrix for later release.”

In one embodiment of the instant invention, an anti-microtubule agent (such as, e.g., paclitaxel), is adsorbed onto the surfaces of the nanoparticles. In one aspect of this embodiment, the release rate of the paclitaxel is varied by cross-linking the carbohydrate matrix after crystallization. As is disclosed in column 4 of U.S. Pat. No. 4,501,726, “It is also possible to vary the release rate of the pharmacologically active substance by cross-linking the matrix after crystallization. The tighter the matrix is cross-linked, the longer are the release times. Different types of cross-linking agents can be used, depending upon whether or not water is present at the cross-linkage. In aqueous environment, it is possible to use, inter alia, divinyl sulphone, epibromohydrin or BrCN. In the anhydrous phase, it is possible to activate with tresyl reagent, followed by cross-linking with a diamine.”

The constructs of U.S. Pat. No. 4,501,726 may advantageously use applicants nanomagnetic particles which provide a superior magnetic moment per unit volume.

By way of further illustration, one may use the delivery system of U.S. Pat. No. 4,652,257 to deliver an anti-microtubule agent (such as paclitaxel) to a site within a human body, such as, e.g., an implanted medical device; the entire disclosure of this United States patent is hereby incorporated by reference into this specification.

Claim 1 of U.S. Pat. No. 4,652,257 describes: “A method of delivering a therapuetic agent to a target site within the body, comprising the steps of: introducing ferromagnetic particle embedded vesicles containing said therapuetic agent into the blood stream upstream of said target site; applying a magnetic field having sufficient strength to immobilize said vesicles at said target site; immobilizing said vesicles at said target site; and oscillating said magnetic field at a rate sufficient to vibrate said ferromagnetic particles such that said vesicles's membrane is destabilized or lysed thereby controlling the rate of release of said therapuetic agent at said target site.” The “ferromagnetic particle” of U.S. Pat. No. 4,652,257 may be replaced with applicants' nanomagnetic particle of this invention.

The lysing of the vesicle by the application of a magnetic field is described at column 5 of U.S. Pat. No. 4,652,257, wherein it is disclosed that: “In the present invention, the vesicles are formed using polymerizable lipids which are subsequently polymerized by exposing the vesicles to ultra-violet light. Using a Rayonet Photochemical Reactor Chamber (model RPR-100), it takes between 5-30 minutes at a UV strength of about 25 watts. Alternatively, the vesicles can be formed from lipid/polymerizable lipid mixtures so as to vary the permability of the vesicle membrane. Once formed, the vesicles, containing the therapeutic agent and ferromagnetic particles, can be injected upstream from the target site. The vesicles migrate through the blood stream to the target area where they can be immobilized by an 8000 gauss magnetic field. Once immobilized, the vesicle's contents can be released by oscillating the magnetic field at a rate sufficient to vibrate the embedded ferromagnetic particles. The total contents of the vesicle can be released by oscillating the magnetic field sufficiently to lyse the membrane. Alternatively, particularly with the mixed lipid/polymerizable lipid vesicle, the contents can be released at a controlled rate by varying the oscillation rate so as to destabilize the membrane making it more permeable to the therapeutic agent but not so as rupture the membrane. The magnetic field can be oscillated at a rate between 10 and 1200 cycles per second but a range between 500 and 1000 cycles per second is prefered. The magnetic field can have any strength necessary to immobilize the vesicles. A range between 5000 and 12000 Gauss is prefered with 7000 to 9000 Gauss being most preferred.” As will be apparent, the lysing of the vesicle will be more readily attained with applicant's nanomagnetic particles, which have superior magnetic moments per unit volume.

In one embodiment, the coercive force and the remnant magnetization of applicants' nanomagnetic particles are preferably adjusted to optimize the magnetic responsiveness of the particles so that the coercive force is preferably from about 1 Gauss to about 1 Tesla and, more preferably, from about 1 to about 100 Gauss.

Some of the therapeutic agents that may be used in the process of U.S. Pat. No. 4,652,257 are described at columns 5-6 of this patent, wherein it is disclosed that: “For example, vesicles containing oncolytic agents could be injected intra-arterially upstream from a tumor, localized in the tumor by the magnetic field, and disrupted by oscillating the magnetic field. The toxicity-of the oncolytic agents is, therefore, confined to the area where the tumor is located. Therapeutic agents which can be encapsulated in the vesicles include hydrophillic materials such as vindesine sulfate, fluorouracil, antinomycin D, and the like. Basically, any known oncolytic agent, anti-inflamatory agent, anti-arthritic agent or similar agent which is hydrophillic can be incorporated into the vesicles.”

In one embodiment of this invention, an anti-microtubule agent (such as, e.g., paclitaxel) is incorporated into the vesicle of U.S. Pat. No. 4,652,257 and delivered to the situs of an implantable medical device, wherein the paclitaxel is released at a controlled release rate. Such a situs might be, e.g., the interior surface of a stent wherein the paclitaxel, as it is slowly relesased, will inhibit restenosis of the stent.

U.S. Pat. No. 4,674,480 also discloses a magnetic drug composition that is “. . . operable in the presence of the body fluid to degrade and release the drug contents of said microcapsules after a time delay once said drug units have entered the body and said drug units are targeted to a select cancer site in the body of the living being to whom said medical dose has been administered” (see claim 9 of the patent). The entire disclosure of this United States patent is hereby incorporated by reference into this specification.

Claim 1 of U.S. Pat. No. 4,674,480 describes one preferred process of this patent. This claim 1 discloses: “A method of effecting a medical treatment or diagnosis, said method comprising: (a) forming a multitude of drug units, each containing a quantity of a drug encapsulated by a carrier material within the drug unit formed, (b) administering a select quantity of said drug units to the body of a living being, (c) allowing at least a portion of said administered drug units to travel through the body to a select location in the body and to become disposed adjacent select tissue at said select location to allow said select tissue at said select location to be treated with the encapsulated drug thereof, and (d) after a substantial quantity of said drug units are so disposed, causing the drug contained in each unit to be released from the carrier material encapsulation and to flow to tissue adjacent which said units are disposed.”

Various means are disclosed in U.S. Pat. No. 4,674,480 for “. . . causing the drug contained in each unit to be released . . . . ” Thus, e.g., in claim 2 of the patent, it is disclosed that “. . . the quantities of drug contained by such drug units are released by causing said encapsulating carrier material of said units to become ruptured to destroy the encapsulating effect.” Thus, e.g., claim 3 of the patent describes a method in which “. . . the quantities of drug contained by said drug units are released from encapsulation by causing said encapsulating carrier material of said drug units to become porous and release drug contained thereby . . . . ” Thus, e.g., claim 4 describes a method in which “. . . the quantities of drug contained by said drug units are released from the drug units by causing said encapsulating carrier material of said drug units to dissolve or biodegrade in body fluid . . . . ” Thus, e.g., claim 5 describes a method in which “. . . the quantities of drug contained by said drug units are released from the drug units by causing said encapsulating carrier material of said units to biodegrade within said living being at a select time after being administered to the body of said living being . . . . ” Thus, e.g., claim 6 describes a method in which” . . . the quantities of said drug contained by said drug units are released therefrom by causing a quantity of a nuclide contained in at least certain of said units to become radioactive and, in so becoming, to explosively destroy at least a portion of the encapsulating carrier material to release the encapsulated drug from the units . . . ” Thus, e.g., claim 7 describes a method in which “. . . a substantial portion of said administered drug units are permitted to travel in the bloodstream of said living being and to flow with the blood of said living being to the tissue of the body to be treated when the drug encapsulated in said drug units is released from encapsulation by said drug units at the site of said tissue . . . . ” Each of these drug releasing methods may be used in the process of this invention to release, e.g., therapeutic agent 18 from a material within which it is disposed or to which it is bound.

Some of the preferred “releasing means” of U.S. Pat. No. 4,674,480 are described in columns 5-9 of such patent.

Thus, and referring to columns 5-6 of U.S. Pat. No. 4,674,480,” . . . a drug unit 10 . . . may comprise one of a multitude of such units disposed in a liquid or capsule which is administered to a living being. The drug unit 10 comprises a bulbous capsule 11, shown as having a spherical or ellipsoidal shape, although it may have any other suitable shape. A side wall 12 completely surrounds contents 15 which may comprise any suitable type of medication such as an organic or inorganic liquid chemical, a plurality of such chemicals, a biological material, such as an antibiotic or a liquid containing one or more living or dead virus, bacteria, antibodies, phages, or other material which is desired to be dispensed within or in the immediate vicinity of disease tissue or disease cells existing within a living being.”

U.S. Pat. No. 4,674,480 then goes on to describe “nuclide particle 14,” stating that: “A small particle 14 is supported against a portion of the outside surface 13 of the wall 12. Particle 14 is a nuclide material, such as boron-10 . . . . Such paricle 14 may comprise a plurality of particles bonded by a suitable resin or other material coating the outside surface 13 of capsule 11. Particle 14 may be rendered radioactive and caused to generate radiation or explode as illustrated in FIG. 2, to rupture a portion of the wall 12 to permit the contents 15 of capsule 11 to flow through the opening 12R. A plurality of openings may be formed in the wall when particles of such nuclide are simultaneously rendered radioactive. Such particle 14 may be so rendered radioactive when the drug unit 10 is disposed or flows to a select location within a living being, such as a location of diseased tissue, dead or calcified tissue or bone desired to be subjected to a chemical or biological agent, such as the contents 15 of the capsule 11.”

U.S. Pat. No. 4,674,480 also discloses that “The contents 15 may be under slight pressure during the formation of the capsule 11 or may be pressurized as the result of the heat or pressure of the radiation generated when the particle or particles 14 become radioactive. Accordingly, one or more of such particles may also be disposed within the body of the contents 15 or against the inside surface of the wall 12 or within such wall for such purpose and/or to render the wall 12 ruptured or porous to permit flow of the contents 15 from the capsule and/or absorption of body fluid into the capsule to mix or react with its contents.”

U.S. Pat. No. 4,331,654 also discloses that “The capsule 11 may vary in size from less than a thousandth of an inch in diameter to several thousandths of an inch in diameter or more, if a multitude of such capsules are utilized to deliver a chemical or biological agent to a particular location within a living being via the bloodstream or by direct injection to such location. It may also comprise a larger capsule which is injested by mouth, inserted by catheter or implanted by of surgery at a select location in tissue or a body duct. Wall 12 may be made of a synthetic polymer, such as a suitable plastic resin, a starch, protein, fat, cell tissue, a combination of such materials or other organic matter. It may be employed per se or in combination with other elements as described hereafter. Similar or differently shaped capsules of the types illustrated in the drawings may be combined or mixed and may contain a plurality of different elements or drugs mixed in each or provided in separate such elements or drugs cooperate in alleviating a malady such as by attacking or destroying bacteria or diseased tissue, improving the condition of living cells, changing the structure of living tissue or cells, dissolving or destroying tissue cells, repairing cells or cell damage, etc.”

U.S. Pat. No. 4,331,654 also discloses that “In FIG. 3, a drug unit 20 of the type shown in FIGS. 1 and 2, comprises a spherically shaped container or shell 21 of one or more of the materials described with a spherical sidewall 22. The outer surface 23 may contain one or more particles of a nuclide of the type described and/or one or more antibodies, such as monoclonal antibodies, attached thereto by a suitable resin or assembled with the container 21 by a suitable derivatizing agent. Disposed within the hollow interior of spherically shaped container 21 is a liquid material or drug 25 having one or more particles 24 of a nuclide or a plurality of nuclides floating or supported therein. Such nuclide or nuclide particles 24 may be rendered radioactive, as in FIG. 2, by directing a beam or beams of neutrons at the drug unit 20, such a neutron beam source may be located outside the body in which the drug units are disposed. The neutrons render the one or more particles 24 radioactive in a manner to either explode or generate sufficient radiant energy to cause the liquid contents 24 to at least partially evaporate or otherwise expand in a manner to force such contents through the wall 22, which may be porous or rendered porous or may be ruptured by the internal pressure effected when the particle or particles 24 become radioactive. In such a manner, the contents 25 may be completely or partially expelled from the container and applied to adjacent or ambient tissue or disease matter located within a human living being adjacent the drug unit 20. In a particular form of FIG. 3, one or more particles of a nuclide disposed on the outer surface 23 of the wall 22 may be rendered radioactive and explode to rupture a portion or portions of the wall, rendering same porous or providing an opening therein or destroying such wall so that the contents 25 may flow therefrom to surrounding material.”

U.S. Pat. No. 4,331,654 also discloses that “In FIG. 4 is shown a modified form of drug unit 30 formed of a capsule 31 of the type illustrated in FIGS. 1 and 2 or 3. A sperhical or ellipsoidally shaped sidewall 31 completely surrounds a liquid, cream or solid drug or chemical 33 having one or more particles 34 of a nuclide . . . . Bonded or otherwise attached to a portion of the exterior surface 32 of wall 31 is an antibody 36, such as a monoclonal antibody, which is targeted to a specific antigen located within a living being. Such antigen may comprise, for example, the surface of a cancer cell, bacteria, disease tissue or other material desired to be affected by the chemical or agent 33 released from the drug unit 30 when the nuclide particle or particles 34 located within the contents 33 or disposed within or against the surface 32 of the wall 31 of the capsule, are rendered radioactive and explode or generate sufficient heat or radiation to effect one or more of the described actions with respect to the wall 31 of the capsule, such as render same porous or ruptured. A polymer or other derivatizing agent 35 is employed to bond the antibody or monoclonal antibody 36 to a portion of the surface 32 of the capsule.”

U.S. Pat. No. 4,331,654 also discloses that “In FIG. 5 is shown a modified form of FIG. 4 wherein a drug unit 40 is composed of a base unit or container 41 which is illustrated as a porous spherical body, the cells 43 of which contain a drug or chemical dispensed therefrom to surrounding fluid or tissue. One or more particles 44 of a nuclide of the type described above, are disposed within the body of the spherical container 41 and/or against the outside surface thereof to be rendered radioactive when a beam or beams of radiation, such as neutrons, are directed thereat. The radiation is absorbed by the particle or particles to effect such radioactivity which may comprise explosive and/or nonexplosive radiation. Thus, liquid or particulate drug material (1) may be forced from the cells of the container 41, (2) effect a chemical reaction resulting in such action or (3) partially or completely destroy the container 41 to release its contents.”

U.S. Pat. No. 4,331,654 also discloses that “A plurality of antibodies 45 as disposed against and bonded to the outside surface 42 of the container 41. In this embodiment, monoclonal antibodies 45 are targeted to a particular antigen, such as a disease or cancer cell or other cell located within the body of a living being to be treated, destroyed or otherwise affected by the action of chemical or biological agent carried by the container 41 and, if so constructed, by the radioactivity generated when the nuclide particle or particles 44 are rendered radioactive as described.”

U.S. Pat. No. 4,331,654 also discloses that “In FIG. 6 is shown a container assembly 50, which may be a preformed capsule or otherwise shaped implant having a container body 51 with a suitable sidewall 52 and having contents 56, such as one of the chemicals or biological agents described above, which contents are desired to be dispensed from a neck portion 53 of the container. Supported within the neck portion 53 is a solid material 54 containing one or more particles 55 of a nuclide of the type described. When such particle or particles 55 are rendered radioactive by externally applied radiation, they may heat and melt the material 54 or explode and rupture such material and a portion of the neck 53 of the container. Thus, contents 56 flow from container 50, either by capillary action if the neck 53 is of a capillary construction, by internal pressure created by the heat of radiation or existing within the container, by gravity or osmosis effected when the wall 52 of the container and/or the filling material 54 is rendered porous or when porous filling material 54 is exposed to the exterior of the container when a portion of the neck wall 52 neck is ruptured or destroyed when a particle or particles 55 become radioactive.”

U.S. Pat. No. 4,331,654 also discloses that “In FIG. 7 is shown a portion of a container 60 having a sidewall 61 and a plurality of interior wall portions 65 extending completely through the container to provide a plurality of separate chambers 66. Each chambers 66 may contain different portions of the same chemical or biological agent or different chemicals or biological agents. Disposed against select portions of the sidewalls 61 and either bonded to the exterior surface 62 of the container 60 or supported within a material 63 coating of such sidewall, are a plurality of particles 64 of a nuclide. In FIG. 7, one particle 64 is shown aligned with each chamber 66 of although a multiple of such particles may be so aligned and disposed. When a beam or beams or radiation, such as neutrons, are selectively directed at selected portions of the sidewall 61 and the particle or particles 64 aligned therewith, the selected portions of the sidewall may be ruptured, rendered porous or have small openings formed therein when the particle or particles of nuclide are rendered active as described. Thus, contents 67 are selectively disposed when the sidewall portions of the chamber or chambers 66 are ruptured or rendered porous when the selected nuclide particle or particles become radioactive.”

U.S. Pat. No. 4,331,654 also discloses that “Nuclides will provide miniature explosive atomic reactions capable of rendering microcapsules such as liposomes, starch, protein or fat microballoons in the order of one to ten microns or greater in diameter porous or ruptured to release their liquid medication contents to surrounding tissue or cells, may include boron-10, cadmium-113, lithium-6, samarium-149, mercury-199, gadolinium-155 and gadolinium-157. Nuclides which may be attached or coated on or disposed within the described microcapsules for diagnostic and indicating purposes include such radioactive elements as cobalt 57; galium 67, cesium 131, iodine 131, iodine 125, thalium 201, technicium 99m, indium 111, selenium 75, carbon 11, nitrogen 13 or a combination of such radioactive elements. In a particular form of the invention, both a neutron activated and atomically explosive particle or particles, such as atoms, of a nuclide and a normally radioactive nuclide of the groups above may be provided in a single drug unit per se or in combination with a chemical as described.”

U.S. Pat. No. 4,690,130 discloses a process in which electromagnetic radiation is selectively applied to a patient in every area except for a “treatment zone”; the entire disclosure of this United States patent is hereby incorporated by reference into this specification. Thus, and as is described in claim 1 of such patent, there is provided a method for “. . . A method for applying a therapeutic agent to a treatment zone in a patient, which treatment zone is not adjacent the skin of the patient, comprising: applying a steady or low frequency magnetic field to the patient to include the treatment zone; supplying microspheres for circulation through the patient to include said zone, said microspheres including a therapeutic agent, and also includes medically bodily compatible magnetic material having a Curie point at which the magnetic material becomes substantially non-magnetic slightly above the normal body temperature of the patient; and applying high frequency electromagnetic field energy to said patient where said magnetic field is applied to said patient, except to said treatment zone, to heat up said magnetic material to demagnetize it so the microspheres are not restrained by said magnetic field except in said treatment zone.”

The rationale for the invention of U.S. Pat. No. 4,690,130 is described in column 3 of such patent, wherein it is disclosed that “. . . the present invention involves the selective restraint of magnetic material having an accessible Curie point temperature, and the use of (1) a magnetic field to hold the magnetic material and (2) the use of a high frequency electromagnetic field to selectively heat the magnetic particles to a temperature above the Curie point. In order to effect restraint of particles within a selected field zone, two conditions must be simultaneously met therein—(1) the particles must be magnetically responsive i.e., at a temperature sufficiently below the Curie point to exhibit substantial ferromagnetic exchange coupling, and (2) the static magnetic field gradient must be of adequate strength to restrain magnetically responsive particles within capillary vessels in the selected field zone. It is necessary and sufficient that either one of these conditions be absent at sites external to the selected field zone (where it is desired to concentrate the microspheres) in order to effect free unrestrained flow of the particles. The appropriate presence and absence of these conditions is regulated by the geometrical intersection of an oscillatory electromagnetic field and the static magnetic field, as set forth below. The effect of the oscillatory electromagnetic field is to heat up the magnetic particles and render them substantially nonmagnetic.” The process of U.S. Pat. No. 4,690,130 may be used to heat the nanomagnetic material

U.S. Pat. No. 4,690,130 also discloses that “It is a general feature of this invention that the oscillatory electromagnetic wave intensity be absent or of negligible value in the selected target zone. Oscillatory electromagnetic waves may be locally diminished (1) by natural exponential attenuation upon passage through lossy material, and (2) cancellation of waves oppositely phased emanating from two or more sources.”

In the section of U.S. Pat. No. 4,690,130 appearing at column 6 thereof and relating to “ENERGY ABSORPTION IN PARTICLES,” it is disclosed that: “A central feature of this invention is the spatially controlled disposition of oscillatory electromagnetic energy in said particles. In an idealized circumstance, such energy disposition would be zero at the targeted field zone and abruptly very high elsewhere. Specific physical interactions mediate to diminish the abruptness of the absorption transition in and out of the target field zone. However, using the techniques as described herein, together with materials having appropriate absorption characteristics and moderately abrupt Curie temperature, effective restraint in the target zone is achieved.”

U.S. Pat. No. 4,690,130 then goes on to discuss absorption phenomena, stating that(at column 6 et seq.) “The absorption of oscillatory electromagnetic radiations in magnetic and in conductive matter will now be considered. For example, from the American Institute of Physics Handbook (McGraw-Hill, New York, 1957), Sec. 5 p. 90, tin and magnetic iron have very similar conductivities, being in a ratio of 1:1.2. Nevertheless, the absorption of energy flux is in a ratio of 1:16 based upon the relative penetration depths at which the flux has diminished to l/e squared for radiation in the range of 1 to 3000 MHz. This rather marked absorption difference is attributed to the relative magnetic permeabilities which are in a ratio of 1:200. Electromagnetic radiation, which consists of oscillatory electric E and magnetic B vector components, is absorbed in relation to electric conductivity and magnetic permeability, respectively. Accordingly, it may be understood that tin and magnetic iron both absorb a certain similar proportion of the electric component but the magnetic iron additionally absorbs a very large proportion of the magnetic component. If both components are radiated at equal amplitudes, it may be expected that magnetically responsive substances will absorb energy predominantly from the magnetic component.”

U.S. Pat. No. 4,690,130 also discloses that “The relevance of this interaction to the present invention may now be understood. The particles of this invention have a magnetic permeability which is very sensitively temperature dependent. In the targeted field zone, the particles are to be maximally magnetically responsive in order to effect restraint with respect to the static magnetic field. In regions immediately exterior to this zone, the particles are to be minimally magnetically responsive in order to allow unrestrained flow into the zone.”

U.S. Pat. No. 4,690,130 also discloses that “If, for example, the electromagnetic radiation immediately exterior to the zone were ten times as high as in the zone, then the particles would be expected to sustain a ten-fold higher energy absorption and a concurrent temperature rise outside the zone. However, since the particles are deliberately designed to exhibit a substantial reduction in magnetic permeability in response to a substantial temperature rise, the absorption of the magnetic component of oscillatory electromagnetic energy is severely diminished. If the magnetic component is the predominant source of energy, then the desired effect partially cancels the means to achieve that effect. That is, an initially high temperature rise brought about by a strong absorption of the magnetic component is quickly followed in equilibrium by a partial loss in-temperature as the magnetic component is less strongly absorbed. Since the final equilibrium temperature is not as high as the brief initial temperature, the particles immediately exterior to the zone sustain only a partially reduced magnetic responsiveness and may exhibit a degree of undesired restraint in response to the static magnetic field. Effectively, the minimum size of the targeted field zone is increased somewhat and the concentration of restrained particles is not as abruptly delineated by the zone.”

U.S. Pat. No. 4,690,130 also discloses that “As developed below, however, the multiplicity of antenna elements may be so configured and phased so as to substantially cancel the oscillatory magnetic components and augment the oscillatory electric components in the aforementioned regions exterior to the targeted field zone. Since the interaction of the particles with regard to the oscillatory electric component is effectively independent of temperature, the energy absorption of the electric-enhanced oscillatory field is essentially proportional to the intensity of the field.”

U.S. Pat. No. 4,690,130 also discloses that “This type of arrangement increases the sharp delineation of the particle restraint zone. Specifically, consider FIG. 6 where the instantaneous oscillatory field components are generated from a pair of equally driven antenna dipole elements 52(a) and 54(b). The respective resultant magnetic components Ba and Bb at the point 56 are oppositely oriented, perpendicular to the plane of the page, thereby cancelling. The electric components add vectorially giving a value Etot significantly larger than the components themselves. Extending this configuration to a second pair of antenna elements 58 and 60, where all four elements are on the vertical edges of a box-like geometrical shape of square cross section, as shown in FIG. 7, allows the generation of a strong electric oscillatory field located centrally above as indicated at reference numeral 62. The corresponding net magnetic component remains at a constant zero magnitude.”

In one embodiment of the instant invention, and as described elsewhere in this specification, a multiplicity of nanomagnetic particles and/or nanomagentic coatings are used instead of, or in addition to, the “antenna elements” of U.S. Pat. No. 4,690,130 so that the electromagnetic fields disposed about an implanted medical device (such as, e.g., an implanted stent) cooperate to cause a therapeutic agent to travel into the surface of the stent.

Referring again to U.S. Pat. No. 4,690,130, at columns 7-9, such patent discusses the properties of the particles used in the process of their invention. It is disclosed that: “A number of substances called ferromagnetics, such as iron, may be very strongly magnetized while in the presence of a magnetic field. Most of these substances exhibit magnetization versus temperature curves similar in shape to FIG. 8 but differing in scale. For example, the magnitude of the maximum magnetization Mm and the temperature Tc on the absolute scale varies considerably among the known ferromagnetics. The value Tc is the temperature at which the extrapolated curve intersects the axis, and is known as the Curie point. A substance responding as in FIG. 8 is said to be ferromagnetic when below the Curie point, Tc. At temperatures above the Curie point Tc, the curve descent levels off somewhat wherein a substance is said to be paramagnetic.”

U.S. Pat. No. 4,690,130 also discloses that “The very large magnetization exhibited by ferromagnetic substances is a collective quantum mechanical phenomenon known as exchange coupling. When aggregates of certain atomic species are formed, a very large percentage of the individual atomic magnetic moments align together. The broad gradually sloping region of FIG. 8 below Tc shown in FIG. 8, indicates nearly 100% alignment. As temperature increases up to Tc, this exchange coupling is disrupted by thermal agitation with a concurrent decrease in magnetization. The paramagnetic state, above Tc, is said to exist when sufficient disruption occurs such that the coupling is totally broken and the atoms act independently in their alignment response. The maximum magnetization Mm for the purposes of this invention, should be substantial, ideally comparable to iron and other strong ferromagnetics. The particles of this invention should also exhibit response wherein human body temperature, which is 310 degrees K., or 98.6 degrees Fahrenheit, should fall at a point TO on the shoulder of the curve at the onset of rapid descent as in FIG. 8. For a value of TO so situated, Tc is typically a modest increment higher on the order of magnitude of 10 degrees Kelvin. While it is not necessary that the induced temperature increase actually reach or exceed Tc, it is essential that a very large relative decrease in magnetization be effected. Nevertheless, substances having Curie points slightly above 310 degrees K. are indicative of good candidates for the particles.”

U.S. Pat. No. 4,690,130 then goes on to disclose that: “Pure iron for example is inappropriate, having a Curie temperature of 1040 degrees K. Several possible choices and their Curie temperature in degrees Kelvin include, CrTe, 320; Cr3 Te4, 325; Nd2 Fe7, 327; Ni—Cr (5.6% atomic % Cr), 324; and Fe—Ni (about 30% Ni) 340 as well as many other combinations. Furthermore, it is known in the art that small percentage variations in composition can increase or decrease the Curie temperature by several degrees. For instance, the Fe—Ni alloy can be altered to provide a lower Curie temperature of perhaps 320. The Fe—Ni alloy is also desirable since it is a moderately good conductor, essential to absorption of the oscillatory electric component. Fe—Ni also exhibits magnetization comparable to that of pure iron, Fe. Biologically, the elements Fe and Ni do not exhibit the undesirable toxicity common to an element such as chromium, Cr, included in some of the afore-mentioned combinations, and the material is therefore substantially medically inert.”

In the process of U.S. Pat. No. 4,690,130, an “oscillatory wave generator” is used to raise the temperature of some of the particles used in such process. As is disclosed at lines 63 et seq. of column 8 of such patent, “The purpose of the oscillatory wave generator is to significantly raise the particle temperature in regions exterior to the targeted zone. The temperature rise is caused by the preferential conversion of electromagnetic energy to thermal energy by the particles. Conversely, the temperature of surrounding tissue is not significantly raised when subjected to the same oscillatory waves.” Such an oscillatory wave generator may be used to raise the temperature of the nanomagnetic material of this invention.

U.S. Pat. No. 4,690,130 also discloses that “The underlying physical principles are readily understood in conjunction with the relative absorptivity of good conductors and patient tissue. For example, at 100 MHz, the intensity decreases by a factor 1/e squared in 0.0007 cm of copper and in 7 cm of tissue, indicating that a good conductor such as copper is 10,000 times as absorptive as tissue. The thermal energy of the particles is subsequently dissipated to surrounding tissue. However, the total mass of injected particles is many orders of magnitude less than that of the patient. Consequently, the patient is effectively an infinite heat sink negligibly increased in temperature by the relatively small total heat content transferred from the particles. Thereby, the particles are readily increased in temperature whereas direct and indirect energy transfer to tissue is negligible resulting in an insignificant rise in overall patient temperature.”

U.S. Pat. No. 4,690,130 then discloses (at column 9 et seq.) various devices that may be used to provide the desired oscillatory electromagnetic field. It states that: “The oscillatory electromagnetic field may be provided by devices such as a MA-150 waveguide antenna horn coupled to a BSD-1000 RF power generator, both manufactured by BSD Medical Corporation, Salt Lake City, Utah. These devices are conventionally used to achieve regional hyperthermia by selectively directing radio frequency (RF) electromagnetic waves of high intensity at a tumor site within a patient. Certain tumor types are temperature sensitive compared to normal tissue. In this regard, a temperature increase of about 5 degrees K. sustained for approximately 20 minutes is often effective in killing tumor cells, while normal cells are left undamaged.” One or more of these devices may be used to heat the nanomagnetic material of this invention to a desired temperature.

U.S. Pat. No. 4,690,130 also discloses that “A coaxial conductor cable interconnects the BSD-1000 to a termination within the MA-150 waveguide antenna horn consisting of plate electrodes across a dielectric layer. The antenna horn facilitiates directivity of the projected electromagnet waves. A flexible water bag affixed to the mouth of the antenna horn is pressed against the patient over the site targeted for the application of electromagnetic energy. The water efficiently couples the RF waves into tissue and minimizes reflections. Thermal energy generated in the water is continuously removed by pumping through an ice-filled heat exchanger. By this means, the surface of the patient is cooled through a thermal conductive process which allows for additional control of temperature within the patient.”

U.S. Pat. No. 4,690,130 also discloses that “The BSD-1000 RF power generator provides fully adjustable power from 5 watts to 250 watts over the frequency range of 95 MHz to 1000 MHz. Although heating may be obtained over a wider range, for the purposes of the present invention, a frequency range of about 50 megahertz or 50,000,000 cycles per second, up to about 200 megahertz is preferred. The reason that this range is preferred is that above 50 megahertz, there is more absorption by the particles and less by the human body; and above 200 megahertz, hot spots may develop near the horns. However, effective heating may be accomplished over a much broader range of frequencies.” This BSD-1000 RF power generator may be used to heat the nanomagnetic particles of this invention.

U.S. Pat. No. 4,690,130 also discloses that “More than one MA-150 antenna horn may be driven by the BSD-1000 using power splitters. The MA-150 units may be arranged in an array such that each unit represents an antenna element of this invention. The power output from the BSD-1000 to each MA-150 unit may be phase shifted and attenuated to control of the oscillatory wave intensity as described with respect to this invention. E-field sensors available from BSD are placed in skin contact on the patient to monitor the incident electric field and estimate the resultant internal temperature distribution. The MA-150 horns project electromagnetic waves with the electric and magnetic vectors mutually perpendicular to each other and also to the direction of the wavefront propagation as is common to all such electromagnetic propagation. Thereby, as described hereinabove, two adjacent MA-150 horn units may be placed to produce total cancellation of the magnetic vector and augment the electric vector in the neighborhood of a mid-plane between the units. Correspondingly, opposing MA-150 units produce an intermediate null plane by destructive interference, as described herein, using opposite relative phase.”

U.S. Pat. No. 4,690,130 also discloses that “The component devices used in hyperthermia are necessarily operated at high power levels to produce gross regional temperature increases of about 5 degrees K. in and around targeted tissue. For the purposes of this invention, sub-therapeutic power levels with respect to hyperthermia, are used such that actual regional tissue temperature at all sites is never increased by more than 2 degrees K., and generally by less than 1 degree K. Nevertheless, when such tissue contains particles as described herein, then said particles locally sustain a substantially higher temperature increase of approximately 10 degrees K. as demonstrated by loss of magnetic responsiveness.”

U.S. Pat. No. 4,690,130 also discloses that “Furthermore, the objective of hyperthermia is, ideally, a focal heating of targeted tissue e.g., a tumor. This focal heating may be augmented by constructive interference of horn antennae at the depth of the tumor whereas in the context of the present invention, a significantly reduced RF intensity exists at the targeted tissue. It may be appreciated that attenuation by tissue absorption, and by phase inversion of the electric vectors from opposing horn antennae and destructive interference, or cancellation, may be used to produce this reduced RF intensity.”

U.S. Pat. No. 4,690,130 also discloses that “The static magnetic field may be produced by Model HS-1785-4A DC power supplies combined with circular coil elements such as those in the Model M-4074 assembly, both available from Walker Scientific Inc., Rockdale Street, Worcester, Mass. 01606. The power supply generates 0-85 amps at 0-170 VDC. The coil elements are wound with aluminum foil 6 inches wide with plastic film insulation between the turns. Each wound coil is affixed to a flat aluminum plate by epoxy resin and water channels milled into the plate facilitate cooling of the coil during operation.” One or more of these means may be used to heat the nanomagnetic coatings and/or prticles of this invention as an “electromagnetic radiation source 41” (see FIG. 1A).

U.S. Pat. No. 4,690,130 also discloses that “A concentric pair of such coils with diameters of twenty inches and eight inches provides an effective depth controllable gradient with magnetic strength in excess of 1000 gauss. Each coil is driven by a separate power supply so that current and polarity is individually controllable.” Such concentric pair of coils may be used to heat the nanomagnetic particles of this invention.

U.S. Pat. No. 4,690,130 also discloses that “The magnetic field may be mapped with a gaussmeter such as the Model MG-3D Hall effect unit available from Walker Scientific, Inc. This instrument can measure fields in the range of 10 to 100,000 gauss with an accuracy of ±0.2%.”

In columns 11-12 of U.S. Pat. No. 4,690,130, preparation of the particles used in the process of such invention is discussed. It is stated that: “A large variety of appropriate metallic alloys in powder form are available from manufacturers such as Ashland Chemical Co., P.O. Box 2219, Columbus, Ohio 43216. A comprehensive reference text prepared by R. M. Bozorth lists several hundred alloys and their respective Curie temperatures. Bozorth's references indicate that an alloy such as 70% Fe, 30% Ni has an appropriate Curie temperature. However, the Curie temperature exhibits a very strong compositional sensitivity, increasing several tens of degrees for each additional percent of Ni. Accordingly, commercially supplied powder consisting of approximately 100 Angstrom size particles exhibits a wide dispersion of Curie temperatures. Particles in an appropriate Curie temperature range such as 320±5 degrees K. may be separated from the particles of inappropriate Curie temperature, by the following steps. The particles are first coated with a fluorocarbon suspension agent available from Ferrofluidics Corporation of Burlington, Mass. The resultant ferrofluid is then heated in a water bath to 340 degrees K. A permanent magnet is used to extract those particles from the ferrofluid which are still magnetically responsive. This process is repeated at 5 degree K. cooling increments down to 315 degrees K. Thereby, the singular extraction at 315 degrees K. exhibits the appropriate Curie transition temperature and is retained, the other extractions being discarded.”

U.S. Pat. No. 4,690,130 also discloses that “Senyei and Widder in U.S. Pat. No. 4,247,406 have suggested the use of human serum albumin (HSA) microspheres as carriers of magnetically responsive particles and therapeutic substances such as chemotherapy agents, since HSA is not readily extracted from the blood by the body's defense systems. Thereby, sufficient time is allowed for an externally applied static magnetic field to trap a substantial quantity of such HSA microspheres flowing in the bloodstream. Microspheres for this invention are prepared as described by Widder and Senyei in U.S. Pat. No. 4,247,406 Example I, page 7 except that in place of Fe3 O4, particles, Fe—Ni alloy particles of 320 degrees K. Curie temperature are used.”

By way of yet further illustration, U.S. Pat. No. 4,849,210 discloses a superparagmagnetic contrast agent and its use in imaging a tumor: the entire disclosure of this United States patent is hereby incorporated by reference into this specification. Claim 1 of this patent describes “The method of imaging a tumor in the liver or spleen of a human subject, comprising parenterally administering to the human subject prior to magnetic resonance imaging (MRI) examination an aqueous suspension composed essentially of microspheres having diameters of less than 1.5 microns, said microspheres being composed of a biodegradable matrix material with a particulate superparamagnetic contrast agent therein, said superparamagnetic contrast agent consisting essentially of ferromagnetic particles of not over 300 angstroms diameter, the quantity of said microspheres administered being effective to appreciably reduce the T2 relaxation time of the subject's liver or spleen; (b) delaying the examination until the microspheres have been segregated by the reticuloendothelial system and are concentrated in the liver and spleen; and then (c) carrying out an MRI examination of the liver or spleen by T2 imaging or mixed T1 and T2 imaging to obtain an image in which the normal liver or spleen tissues appear dark and the tumor appears light with distinct margins therebetween.”

The paramagnetic contrast agents of U.S. Pat. No. 4,849,20 are described in columns 3-4 of this patent, wherein it is stated that: “The superparamagnetic contrast agent is used in particulate form, for example, as particles of 50 to 300 Angstroms diameter. Particle size of not over 300 Angstroms provides ferromagnetic iron compounds with the desired superparamagnetic characteristics; namely, enhanced magnetic susceptibility and low residual magnetization. Preferably, the particulate forms are substantially water-insoluble, such as insoluble oxides or salts. The superparamagnetic contrast agent may also be in the form of particles of an elemental metal such as particularly iron particles sized below 300 Angstroms.”

U.S. Pat. No. 4,849,210 also discloses that “A preferred particulate contrast agent is magnetite, which is a magnetic iron oxide sometimes represented as Fe3 O4 (or as FeO.Fe2 O3.) Commercially, fine powders or suspensions of magnetite are available from Ferrofluidics Corporation, Burlington, Mass. The size range of the particles is submicron, viz. 50 to 200 Angstroms. Other water-insoluble superparamagnetic iron compounds can be used such as ferrous oxide (Fe2 O3), iron sulfide, iron carbonate, etc. . . . For purposes of this invention, the microspheres comprise relatively spherical particles consisting of protein, carbohydrate or lipid as the biodegradable matrix for the paramagnetic contrast agent. For effective targeting to the liver and spleen, the microspheres comprising the encapsulated contrast agents should have diameters up to about a maximum size of 8 microns. An advantageous size range appears to be from about 2 to 5 micro diameter. Less than 1.5 micron microspheres can be used as a livery spleen contrast agent (viz. 1.0 micron size), but circulation time is prolonged, that is, fewer spheres will be rapidly taken up by the RES. Microspheres of larger size than 8 microns may be sequestered in the first capillar bed encountered, and thereby prevented from reaching the liver and spleen at all. Large microspheres (viz. 10 microns or more) can be easily trapped in the lungs by arteriolar and capillary blockade. See Wagner et al., J. Clin. Investigation (1963), 42:427; and Taplin, et al., J. Nucl. Medicine (1964) 5:259.” The structures of U.S. Pat. No. 4,849,210 may be used with the nanomagnetic material of the present invention to prepare preferred contrast agents.

U.S. Pat. No. 4,849,210 also discloses that “The matrix material may be a biodegradable protein, polysaccharide, or lipid. Non-antigenic proteins are preferred such as, for example, human serum albumin. Other amino acid polymers can be used such as hemoglobin, or synthetic amino acid polymers including poly-L-lysine, and poly-L-glutamic acid. Carbohydrates such as starch and substituted (DEAE and sulfate) dextrans can be used. (See Methods in Enzymology, 1985, Vol. 112, pages 119-128). Lipids useful in this invention include lecithin, cholesterol, and various charged phospholipids (stearyl amines or phosphatidic acid). Microspheres having a lipid matrix are described in U.S. Pat. No. 4,331,564.” This matrix material may be used with the nanomagnetic material of this invention.

U.S. Pat. No. 4,849,210 also discloses that “Microspheres for use in practicing the method of this invention can be prepared from albumin, hemoglobin, or other similar amino acid polymers by procedures heretofore described in literature and patent references. See, for example, Kramer, J. Pharm. Sci. (1974) 63: 646; Widder, et al., J. Pharm. Sci. (1979) 68: 79; Widder and Senyei, U.S. Pat. No. 4,247,406; and Senyei and Widder, U.S. Pat. No. 4,230,685. Briefly, an aqueous solution is prepared of the protein matrix material and the paramagnetic/ferromagnetic contrast agent, and the aqueous mixture is emulsified with a vegetable oil, being dispersed droplets in the desired microsphere size range. Emulsification can be carried out at a low temperature, such as a temperature in the range of 20-30° C., and the emulsion is then added dropwise to a heated body of the same oil. The temperature of the oil may range from 70 to 160° C. The dispersed droplets in the heated oil are hardened and stabilized to provide the microspheres which are then recovered. When most of the microspheres as prepared, such as 80% or more, have sizes within the ranges described above, they can be used as prepared. However, where substantial amounts of oversized or undersized microspheres are present, such as over 10 to 20% mof microspheres larger than 8 microns, or over 10 to 20% of microspheres smaller than 1.5 microns, a size separation may be desirable. By the use of a series of micropore filters of selective sizes, the oversized and undersized microspheres can be separated and the microspheres of the desired size range obtained.” These microspheres may also be used with the nanomagnetic material of this invention.

U.S. Pat. No. 4,849,210 also discloses that “The microspheres may contain from 5 to 100 parts by weight of the contrast agent per 100 parts of the matrix material. For example, in preferred embodiments, microspheres can contain from 10 to 30 parts by weight of magnetite particles or another superparamagnetic contrast agent per 100 parts of matrix material such as serum albumin.” The nanomagnetic material may replace, e.g., the magnetite particles.

U.S. Pat. No. 4,863,717 describes the use of “stable nitroxide free radicals” as contrast agents for magnetic resonance imaging. The entire disclosure of this United States patent is hereby incorporated by reference into this specification.

Claim 1 of U.S. Pat. No. 4,863,717, which is typical, describes “In an MRI contrast agent which is a liposome having a bound spin label that is subject to reduction, and thus loss of contrast enhancement capability when in a reducing environment, the improvement wherein the liposome incorporates oxidizing means for oxidizing and thereby restoring spin labels that have been reduced” This contrast agent is useful in magnetic resonance imaging (MRI), which is discussed in column 1 of the patent.

As is disclosed in column 1 of U.S. Pat. No. 4,863,717, “Magnetic resonance imaging (MRI) is a powerful noninvasive medical diagnostic technique that is currently in a period of rapid development. Agents which selectively enhance the contrast among various tissues, organs and fluids or of lesions within the body can add significantly to the versatility of MRI.”

U.S. Pat. No. 4,863,717 also discloses that “Liposomes, with compartments containing entrapped Mn-DTPA or some other paramagnetic substance, have been investigated as potential contrast agents for MRI, as described by Caride et al. in Magn. Reson. Imaging 2: 107-112 (1984). Liposomes tend to be taken up selectively by certain tissues such as the liver and are in general nonantigenic and stable in blood. They are used extensively as experimental drug delivery systems, as described by Poste et al. in “The Challenge of Liposome Targeting in Vivo”, Chapter 1, Lipsome Technology: Volume III, Targeted Drug Delivery and Biological Interaction, G. Gregoriadis, Ed., CRC Press, Boca Raton, Fla. (1984). However, where tested for MRI in the past, liposomes have served merely as vessels to contain encapsulated paramagnetic material.” These liposomes may be used to contain/carry the nanomagnetic material of this invention.

U.S. Pat. No. 4,863,717 also discloses that “Owing to their paramagnetic nature and thus their ability to affect the relaxation times T1 and T2 of nearby nuclei, nitroxide free radicals constitute a class of potential MRI contrast-enhancing agents which are not toxic at low dosages. There are many examples of nitroxide-containing phospholipids, but these are invariably used in low concentrations merely to dope non-paramagnetic phospholipids for biophysical spin labeling studies, as described, for example, by Berliner, L. J., ed., in Spin Labeling: Theory and Applications, Academic Press, New York, volumes 1 and 2, 1976 and 1979 and by Holtzmann, J. L. in Spin Labeling Pharmacology, Academic Press, New York, 1984. European patent publication EP A 0160552, suggests that free radicals such as organic nitroxides may be enclosed within liposomes. The liposomes are said to be sufficiently leaky to water that, although the paramagnetic material is trapped inside, relaxation of bulk water can nevertheless occur by exchange of bulk water with inside water.”

U.S. Pat. No. 4,863,717 also discloses that “A more direct and reliable approach would be to incorporate nitroxide into the bilayer of the liposome. But, one would expect such a use of nitroxide to be hampered by a tendency of the paramagnetic nitroxyl group to accept an electron from the local environment and thus be reduced to a useless diamagnetic N-hydroxy compound, as described in Griffeth et al., Invest. Radiol. 19: 553-562 (1984); Couet, Pharm. Res. 5: 203-209 (1984); and Keana et al., Physiol. Chem. Phys. and Med. NMR 16: 477-480 (1984).”

U.S. Pat. No. 4,863,717 also discloses that “In the past, “reduction” problems have been handled by injecting large amounts of conventional nitroxide compounds into a subject with the intent of “swamping” the reduction reaction. Particularly large dosages have been required because there has been no practical way to direct nitroxide to specific tissues other than the liver and spleen. Because such nitroxides are rapidly diluted in body circulatory liquid, massive amounts of the contrast agent must be administered or the dilution effect renders the nitroxides ineffective as general contrast enhancers. The use of large dosages is not only wasteful and expensive, but also the large quantities of nitroxides and their metabolites can cause toxicity problems in sensitive subjects.”

U.S. Pat. No. 4,863,717 also discloses that “It would be helpful to target certain tissues, say cardiac tissue or tumor tissue, for contrast enhancement. If nitroxides could be concentrated in certain areas of the body, they would encounter fewer “reducing equivalents” than they would if carried throughout the entire body. To accomplish targeting, one thinks in terms of labeling an antibody or monoclonal antibody which seeks out the target tissue. But, it is clear that one or even a few nitroxides attached to an antibody will not provide enough enhancement. On the other hand, one cannot simply add hundreds directly to the antibody because that would almost surely destroy the antibody's ability to bind selectively to its target. Thus, a specific need has been to find a nontoxic contrast enhancing agent that can be targeted for specific tissues.”

“Prior patent publications such as EP A 0160552 and GB 2137612 describe the combined use of a contrast agent and a targeting agent such as an antibody. Such references do not, however, suggest how such targeting agents may be employed effectively with a nontoxic contrast agent such as a compound which effectively employs nitroxide free radicals.”

Two solutions are presented to the “nitroxide reduction” problem described in U.S. Pat. No. 4,863,717. One of these solutions is described at lines 56 et seq. of column 2 of the patent, wherein it is suggested “ . . . to administer a relatively snall number of large molecules, such as arborols, or assemblies of molecules such as liposomes, that have surfaces covered with numerous persistant nitroxide free radicals. The reduction problem is thus addressed through the sheer number of nitroxides on a given molecule.”

This solution is also described at lines 40 et seq. of column 8 of the patent, wherein it is disclosed that: “A second embodiment of the invention employs large molecules, particularly polymeric molecules, or assemblies of molecules, particularly liposomes, constructed to have numerous, i.e. at least about ten, persistent nitroxide free radicals. Because there are so many persistent nitroxide free radicals, the reduction of a few such free radicals is of little significance. Such large molecules or polymers are not merely carriers of encapsulated contrast agents. They are, themselves, the contrast agents since their surfaces are covered with persistent nitroxide free radicals.”

“One such construction is a nitroxide-doped liposome formed by sonication of amphipathic molecules having persistent nitroxide groups. A suitable amphipathic molecule has a polar head group, at least two chains and a nitroxide group sufficiently near the head group that the nitroxide can contact bulk water when in a liposome. As a general rule, the nitroxide must be ten carbons or less from the head group for there to be effective bulk water contact. Particularly well suited are double chain amphipathic molecules having a nitroxide group near the polar end of each chain. To be effective as a sustained use contrast agent, substantially all the amphipathic molecules that make up the liposome should cntain at least one nitroxide group. Most advantageously, the polar head group will also have at least one nitroxide.”

In one embodiment of the instant invention, a therapeutic agent is modified such that it contains a multiplicity of either “persistent nitroxide free radicals” and/or “reversibly reducible nitroxide groups.” In one preferred aspect of this embodiment, the therapeutic agent so modified is an anti-microtubule agent, such as paclitaxel.

By way of further illustration, one may use the hydrophilic microspheres disclosed in U.S. Pat. No. 4,871,716, the entire disclosure of which is hereby incorporated by reference into this specification. As is disclosed in such patent, many of the “prior art” microspheres a hydrophobic. Thus, and referring to column 1 of this patent, “Insoluble magnetically responsive polypeptide or protein microspheres containing therapeutic agents that enable the controlled releases thereof in biological systems following localization by an externally applied magnetic field have generated growing interest in recent years [Widder et al: Cancer Research, 40, p. 3512 (1980) and Widder et al: J. Pharm. Sci., 68, p. 79 (1979)]. Systems utilizing the microspheres have the potential advantage of prolonging effective drug concentrations in the blood stream or tissue when injected thereby reducing the frequency of administration; localizing high drug concentrations; reducing drug toxicity, and enhancing drug stability. Albumin is a preferred protein or polypeptide for the preparation of such microspheres since it is a naturally occurring product in human serum. Although it is usually necessary to cross-link the albumin when preparing microspheres according to conventional methods, cross-linked albumin may still be degraded depending upon cross-link density thereby enabling the use thereof for drug delivery systems, etc.”

“Conventional methods for the preparation of magnetically responsive albumin microspheres are generally of two types. In one method, aqueous dispersions of albumin and magnetically responsive material are insolubilized in vegetable oil or isooctane or other hydrocarbon solvent by denaturing at elevated temperatures (110°-165° C.). Another method involves chemical cross-linking of the aqueous dispersion of albumin at room temperature. Typical of these two types of methods are those described in U.S. Pat. Nos. 4,147,767; 4,356,259; 4,349,530; 4,169,804; 4,230,687; 3,937,668; 3,137,631; 3,202,731; 3,429,827; 3,663,685; 3,663,686; 3,663,687; 3,758,678 and Ishizaka et al, J. Pharm. Sci., Vol. 20, p. 358 (1981). See also U.S. Pat. Nos. 4,055,377; 4,115,534; 4,157,323; 4,169,804; 4,206,094; 4,218,430; 4,219,41 1; 4,247,406; 4,331,654; 4,345,588; 4,369,226; and 4,454,234. These methods, however, result in the formation of relatively hydrophobic microspheres which usually require a surfactant in order to disperse a sufficient quantity thereof in water or other systems for administration to a biological system to ensure the delivery thereto of an effective amount of any biologically active agent entrapped therein. In addition, the hydrophobic nature of conventional polypeptide microspheres make it difficult to “load” large quantites of some water soluble biologically active agents or other material within the microspheres after synthesis. It is an object of the present invention to provide more hydrophilic magnetically responsive polypeptide microspheres which will accept high “loadings” of biologically active substances of other materials especially by addition of such substances after microsphere synthesis, and to prepare such drug loaded microspheres which do not require the utilization of surfactants to enable the preparation of highly concentrated dispersions thereof.”

A method for preparing such “ . . . hydrophilic magnetically responsive polypeptide microspheres . . . ” is described in claim 1 of U.S. Pat. No. 4,871,716. This claim describes: “A method of preparing novel hydrophilic, magnetically responsive microspheres consisting essentially of cross-linked protein or polypeptide particulate and a magnetically responsive material comprising (a) providing a dispersion of an aqueous solution or dispersion of polypeptide or protein microspheres and a particulate magnetically responsive material in an organic, substantially water immiscible solvent solution of a high molecular weight polymer, said organic solvent being substantially a non-solvent for said microspheres and said polymer solution stabilizing the dispersion of microspheres and magnetically responsive material, (b) incorporating a polyfunctional cross-linking agent for said protein or polypeptide in said dispersion, and (c) allowing said cross-linking agent to react with said protein or polypeptide microspheres for a time sufficient to cross-link at least a portion of the microspheres, thereby providing magnetically responsive microspheres containing free reactive functional groups.”

With these hydrophilic moieties, various drugs can be incorporated into the microspheres. Thus, as it disclosed at lines 17 et seq. of column 32 of U.S. Pat. 4,871,716, “The magnetically responsive microspheres of the present invention, unlike those of the prior art are hydrophilic and may be readily dispersed in aqueous media for injection without the need for surfactants. In addition, they may be readily prepared with the incorporation of very high concentrations of therapeutic agents such as the cancer chemotherapeutic drug adriamycin (up to 50 wt % drug). Previous magnetically responsive hydrophobic albumin microsphere-drug preparations have usually succeeded in incorporating not more than 10-15 wt % of such anti-tumor drugs. Also, the hydrophobic magnetically responsive albumin microsphere preparations known in the art have been compromised by a larger dispersion of sizes, limiting the smallest practical size to μm. In contrast, the method of the present invention enables the preparation of particles as small as 80 nm with a narrow distribution of size.” These magnetically responsive microspheres may be incorporated, e.g., in the polymeric material 14.

As is also disclosed in U.S. Pat. No. 4,871,716, “Using a polypeptide cross-linking agent such as glutaraldehyde, reactive aldehyde groups are available on the microspheres for additional chemical reaction. The microspheres may be reacted with amino group containing drugs for covalent coupling, or with the amino acid glycine to enhance hydrophilicity, or coupled covalently to such large protein molecules as lectins, enzymes or antibodies to modify the microsphere surface properties or to provide a carrier system for the coupled proteins. Coupling antibodies to the magnetically responsive microspheres provides methods for the selective removal of cells from cell cultures in suspension by targeting the microspheres to the surface of specific cells, rendering them magnetic, and pulling the cell-microsphere conjugate from solution by means of an externally applied magnetic field, or for use in vivo as a diagnostic aid. Antibodies coupled to magnetically responsive submicron microspheres applied in vivo, i.e., injected intra-arterially, intra-veinously, intra-lymphatically, etc., may localize the microspheres on the surface of specific cells providing a radiopaque element for either radiographic imaging or, magnetic resonance imaging. One type of magnetically responsive microspheres currently used for separation of cell culture suspensions are made of polystyrene which gives a relatively unreactive surface to which antibodies can only be coupled by passive adsorption. As a result, the antibodies tend to dissociate from the microsphere surface with time, necessitating the use of excessive amounts of antibodies and limiting the useful storage life of the microsphere.”

As is also disclosed in U.S. Pat. No. 4,871,716, “The present invention enables the incorporation into the magnetically responsive hydrophilic microspheres of various drugs for localization by means of an extracorporeally applied magnetic field and controlled release, radiographic and magnetic resonance imaging, and selective separation of cell culture suspensions. Various synthetic drugs or enzymes or antibodies or proteins may be incorporated into the microsphere by physical association, by electrostatic interactions, or covalently for altering release kinetics and other property modifications. Such microspheres may also be used for adjuvant compositions incorporating such immunostimulants as interferon or MDP. Albumin may also be combined with various other macromolecules or polypeptides in the course of preparation of the microsphere. For example, polyglutamic acid has been incorporated into magnetically responsive HSA microspheres to enhance the anionic nature of the microsphere and so facilitate the binding of high concentrations of cationic drugs such as adriamycin, bleomycin, or streptomycin. The drugs which may be used in such microspheres include the clinically important antitumor drugs (e.g., adriamycin, mitomycin, bleomycin, etc.) as well as hormones such as cortisone derivatives and antibiotics such as gentamycin, streptomycin, penicillin, etc.”

At columns 16-17 of U.S. Pat. No. 4,871,76, the rate at which the microspheres of this patent release the therapeutic agents to which they were bound was measured. In the experiments described in Tables 8, 9, 10, and 11, e.g. (see columns 17 and 18), release rates of the drug varied from about 19 percent to about 50 percent over a period of from about 2 to about 14 hours.

In one embodiment of this invention, the anti-tumor agent used with the microspheres is paclitaxel, and the drug composition so produced is situated near a drug eluting stent and caused to release such paclitaxel to such stent.

By way of yet further illustration, one may use the magnetic drug assembly described in claim 12 of U.S. Pat. No. 5,411,730, the entire disclsoure of which is hereby incorporated by reference into this specification. Such claim 12 is indirectly dependent upon claim 1 of such U.S. patent, which claim describes: A composition comprising particles of an iron oxide and a polymer, said iron oxide being superparamagnetic, the ratio of polymer to iron being 0.1 to 0.5 (w/w), said particles having sedimentation constants in the range of 150-5000 S, said particles having at least one of the following magnetic properties:a) specific power absorption rate (SAR) greater than 300 w/g Fe, measured in an electromagnetic field of 1 MHz frequency and 100 Oe field strength; b) initial magnetic susceptibility greater than 0.7 EMU/gFe/Gauss; and c) magnetic moment greater than 10-15 erg/Gauss.” Claim 9, which is directly dependent upon claim 1, further specifies that the particles comprise a particle-encapsulating lipid. Claim 12, which is dependent upon claim 9, further specifies hat the particle-encapsulating lipid comprises a therapeutic agent.

At column 3 of U.S. Pat. No. 5,411,730, a discussion of the use of heat to induce the rapid release of pharmaceuticals to a desired site is presented. As is disclosed in this patent, “A different approach to drug targeting has been developed in the works by Yatvin et al. [42,43] and Huang et al. [44]. They used heat to induce rapid release of pharmaceuticals from thermosensitive liposomes composed of phospholipids having transition temperatures slightly above normal physiological temperature. Local hyperthermia, heating of the target area to a temperature of 42°-44° C., would cause the liposome lipids to “melt”, and the liposomes flowing through the vascular bed of a hyperthermized area would rapidly release the entrapped drug into the surrounding medium. Since the drug is released in its intact form, the problems concerning drug extravasation and activity are avoided. So, in the approaches proposed by Yatvin and Huang, the targeted mode of drug delivery substantially depends on the ability to apply hyperthermia a to the area of pathology in a targeted manner; unfortunately, none of the existing techniques of hyperthermia offers a general and satisfactory way to do so.” One may use the nanomagnetic material of applicants' device 10 and cause it to heat to release drugs from liposomes disposed on or in the assembly 10.

In one embodiment of the invention of U.S. Pat. No. 5,411,730, the patentees incorporated adriamycin into thermosensitive ferroliposomes and caused the release of such an anti-tumor agent by electromagnetic radiation. Thus, as is disclosed in column 20 of the patent, “Adriamycin (doxorubicin hydrochloride) is of great interest as a targeted anticancer drug because the great therapeutic potential of this anticancer drug is limited by its systemic toxicity, especially cardiotoxicity [54]. Thermosensitive ferroliposomes are loaded with adriamycin using the “remote loading” technique [55]. This technique employs the property of weak lipophilic bases or acids to cross the liposomal membrane in response to transmembrane gradient of pH [56]. Adriamycin, a weak base, spontaneously accumulates in the liposomes with an acidic (pH 4) interior when the exterior buffer is kept at pH 7 or higher. The accumulated drug remains inside liposomes until the transmembrane pH gradient is fully relaxed. Specifically, we prepare ferroliposomes using glutamate buffer at pH 4.6 (interior) and pH 7.5 (exterior) as described for regular DPPC liposomes [55]. The liposomes are incubated with adriamycin at approx. 0.1:1 drug to lipid ratio, aliquots are taken at various incubation times, and liposome-bound adriamycin is determined by its intrinsic fluorescence in the void volume fraction after passage of an aliquot through a small gel-filtration column (NP-10, Pharmacia). If the incubation time required for the loading is too high, which is not unlikely for a phospholipid bilayer below its transition temperature, we perform incubation at temperature above Tc and quench the drug-loaded liposomes by injecting them into the ice-cold buffer. These experiments establish the incubation time and temperature for efficient loading of the thermosensitive ferroliposomes with adriamycin. The unbound drug is removed from the loaded ferroliposomes by gel filtration through Sephadex G-25. 5. Spontaneous and RF-field triggered release of Adriamycin from thermosensitive ferroliposomes.” One may replace the ferroliposomes with liposomes containing nanomagentic material.

As is also disclosed in U.S. Pat. No. 5,411,730, “We compare the release of adriamycin from thermosensitive ferroliposomes in the physiological saline buffer (PBS), PBS +10% fetal calf serum (FCS), and RPMI 1640 cell culture medium +10% FCS under the following conditions: (a) storage at room temperature and +4° C.; (b) water bath heating to temperatures above Tc; (c) exposure to RF electromagnetic field.”

As is also disclosed in U.S. Pat. No. 5,411,730, “This part of the work explores triggering cell death by exposure of cancer cells to RF electromagnetic field in the presence of Adriamycin-loaded thermosensitive ferroliposomes. We use Adriamycin-sensitive human small cell lung cancer cell lines SHP-77 and H345, routinely maintained in our laboratory. The cells are grown in RPMI 1640 medium plus 10% FCS at 37° C. Ferroliposomes and Adriamycin stock solution are diluted with cell medium and sterilized by filtration. Various doses of sterile ferroliposomes and/or Adriamycin, free or ferroliposome-incorporated, are added to the cells in standard cell-culture 96well plates. To observe the effect of RF field, cell suspension is temporarily transferred to a tissue culture plastic tube inserted into the inductor coil. Growth of the cells is evaluated using our routine (3 H)Thymidine incorporation assay [57]. Table 8 describes the experimental design for this study.” One may substitute nanomagnetic material for the iron material the ferroliposomes.

As is also disclosed in U.S. Pat. No. 4,871,716, “The need for site-specific cancer chemotherapy is evident, and the success in this area is still far below this need. This invention includes a totally novel approach to site-specific chemotherapy. The chemotherapeutic substance is incorporated into thermosensitive liposomes together with ferromagnetic microparticles. Such liposomes normally retain their contents for a long time. However, when such liposomes approach the target site exposed to the source of radiofrequency electromagnetic field, the field heats the ferromagnetic particles; they in turn heat the liposome membrane to reach the transition temperature of the lipid and rapidly release the drug into the vascular bed of the target area. The applications of this approach are multifold. Apart from adriamycin, it is possible to use other anticancer pharmaceuticals in the RF field-dependent ferroliposomal targeted delivery as described here. Such important anatomical areas as head, neck, extremities, and skin are very suitable for RF-field application and therefore for the targeted chemotherapy using the described approach; and the recent development of endoscopic RF-field applicators [58] substantially expand this list to include sites close to the walls of body cavities. It indicates that the approach is practical for its final destination., treatment of human patients.”

In one embodiment of the instant invention, “ . . . other anticancer pharmaceuticals . . . ,” such as, e.g., paclitaxel, are incorporated into the magnetic, thermosensitive liposomes of U.S. Pat. No. 5,41,730 and used to deliver, e.g., paclitaxel to a desired site within a biological organism. In this embodiment, the nanomagnetic film described elsewhere in this specification is utilized.

U.S. Pat. No. 5,441,746 discloses a “wave absorbing magnetic core particle” which is especially adapted to increase its temperature in vivo in response to an external magnetic field and thereby preferentially kill cancer cells; the entire disclosure of this patent is hereby incorporated by reference into this specification. Claim 1 of this patent describes: “A composition comprising a wave absorbing magnetic core particle wherein said magnetic core particle comprises an oxide of the formula M₂ (+3)M(+2)O₄ wherein M(+3) is Al, Cr or Fe, M(+2) is Fe, Ni, Co, Zn, Ca, Ba, Mg, Ga, Gd, Mn or Cd, in combination with an oxide selected from the group consisting of LiO, CdO, NiO, FeO, ZnO, NaO, KO and mixtures thereof, characterized in that said core is capable of adsorbing or coordinating with a hydrophilic moiety, coating with a first amphipathic organic compound, characterized in that said first amphipathic organic compound contains a hydrophilic moiety and a hydrophobic moiety and the hydrophilic moiety is adsorbed or coordinated with the core and the hydrophobic moiety thereby extends outwardly from the inorganic core and further coated with a second amphipathic organic compound wherein said second amphipathic compound contains hydrophobic and hydrophilic moiety and the hydropholic moiety associates with the outwardly extending hydrophobic moiety of said first amphipathic compound to form said wave absorbing composition”

U.S. Pat. No. 5,753,477 discloses a process for transfecting cells which utilizes an external magnetic field. Thus, e.g., claim 1 of this patent describes:” A method for delivery of a composition to cells in vitro, said composition comprising a plurality of substance-carrying superparamagnetic microparticles, comprising: applying a magnetic field in a least two pulses to said composition and cells, wherein said magnetic field is 0.5-50 Teslas in strength, 0.001-200 milliseconds in duration, and insufficient to heat-kill said cells, wherein said magnetic field is applied so as to achieve penetration of the cell membrane by said substance-carrying superparamagnetic microparticles, and said cells are maintainable in viable culture post-delivery.”

The process claimed in U.S. Pat. No. 5,753,477 is related to other “prior art” means for delivering substances into cells, which are discussed incolumns 1 and 2 of U.S. Pat. No. 5,753,477. As is disclosed at lines 30 et seq. of such column 2, “Other previous substance delivery methods have included the use of magnetic nicrospheres to deliver substances into cells. For example, Widder et al. have described the development of a magnetically responsive biodegradable magnetic drug carrier with the capacity to localize both carrier and chemotherapeutic agent by magnetic means to a specific in vivo target site after systemic administration. Widder et al., 58 Proc. Soc. Exp. Bio. & Med. 141 (1978). The carrier consists of albumin microspheres 0.2-2 microns in diameter containing both magnetic Fe3 O4 microparticles (10-20 nm in diameter) and a chemotherapeutic agent entrapped in the albumin matrix. This complex can be held in the desired location via an external static permanent magnet. It has been reported that these complexes are internalized by tumor cells in vitro and in vivo following intra-peritoneal (ip) injection, possibly through passive phagocytosis process.”

The rationale for the process of U.S. Pat. No. 5,753,477 is discussed in column 3 of the patent, at lines 49 et seq. It is disclosed in this column 3 that: “In the absence of an applied magnetic field, superparamagnetic microparticles of size 10 to 100 nm in diameters undergo Brownian motion. When an external magnetic field of moderate strength of 100 to 200 gauss is applied, these particles become magnetized and form into small magneto-needles because of its high initial magnetic susceptibility (0.1 to 0.7 emu/gm Fe/Gauss) and relatively low saturation magnetization (80 emu/gm Fe). In the continual presence of applied field, the small needles can undergo needle-needle interactions and coalesce into bigger needles. These needles generally move past one another until their ends join to each other. Moreover, these needles continue to move slowly toward the applied pole surface of the external magnet. When a stronger magnetic field is applied, the needles move much faster toward the applied magnet. In general, because of the short duration (micro- to milli-seconds) of a pulse in a high magnetic field (2 to 50 Teslas), two stages of magnetic induction are required to act on the particles in order for the particles to accelerate to a high enough velocity to penetrate a single cell membrane or multi-cell layers.”

As is also disclosed in U.S. Pat. No. 5,753,477, “First, the superparamagnetic or ferromagnetic microparticles are pre-magnetized with a primary solenoid of 100 to 1000 Gauss briefly for 1 to 10 seconds (although pre-magnetization is not essential for ferromagnetic particles, so long as they are already magnetic) and immediately followed by the secondary high magnetic pulse (2 to 50 Teslas) of 10 to 200 milliseconds produced by a second solenoid, which serves to accelerate the pre-magnetized particles into the target. Also disclosed is a method as above wherein the pulse(s) is 1 microsecond to 200 milliseconds in length. The target and the magnetic microparticles are placed along the Z-axis and at a position of maximum field gradient directly outside of the secondary pulse coil. Since a homogeneous field is not required for the magnetic biolistic process, any coil which produces high field gradients described will function in the present method. Depending on the cell types, ie. single cell or multi-cell layers, single and/or multi-pulses can be applied to the microparticles and the target. In the absence of a high pulsed field device (field strength greater than 2 Teslas), a coil capable of delivering multi-pulses of continuously moderate field strength (0.5 to 2 Teslas) with pulse durations of 10 to 200 milliseconds, can also be used to deliver superparamagnetic and/or ferromagnetic microparticles into a single cell layer. Intervals between pulses should be kept as close as possible. This set up is more suitable for in vitro single cell layer transfection.”

U.S. Pat. No. 6,200,547 claims a magnetically responsive composition comprised of paclitaxel absorbed on its particles; the entire disclosure of this United States patents is hereby incorporated by reference into this specification. Such claim 7 describes: “A magnetically responsive composition comprising: a) a carrier including particles between about 0.5 μm and 5 μm in crossectional size, each particle including a ratio of iron to carbon in the range from about 95:5 to about 50:50 with the carbon distributed throughout the volume of the particle; and b) a therapeutic amount of paclitaxel adsorbed on the particles.”

At columns 1-2 of this patent, “prior art” magnetically responsive compositions were discussed. It was stated in this section of the patent that: “Metallic carrier compositions used in the treatment of various disorders have been heretofore suggested and/or utilized (see, for example, U.S. Pat. Nos. 4,849,209 and 4,106,488), and have included such compositions that are guided or controlled in a body in response to external application of a magnetic field (see, for example, U.S. Pat. Nos. 4,501,726, 4,652,257 and 4,690,130). Such compositions have not always proven practical and/or entirely effective. For example, such compositions may lack adequate capacity for carriage of the desired biologically active agent to the treatment site, have less than desirable magnetic susceptibility and/or be difficult to manufacture, store and/or use.

As is also disclosed in U.S. Pat. No. 6,200,547, “One such known composition, deliverable by way of intravascular injection, includes microspheres made up of a ferromagnetic component covered with a biocompatible polymer (albumin, gelatin, polysaccharides) which also contains a drug (Driscol C. F. et al. Prog. Am. Assoc. Cancer Res., 1980, p. 261).”

As is also disclosed in U.S. Pat. No. 4,871,716, “It is possible to produce albumen microspheres up to 3.0 μm in size containing a magnetic material (magnetite Fe3 O4) and the anti-tumoral antibiotic doxorubicin (Widder K. et al. J. Pharm. Sci., 68:79-82 1979). Such microspheres are produced through thermal and/or chemical denaturation of albumin in an emulsion (water in oil), with the input phase containing a magnetite suspension in a medicinal solution. Similar technique has been used to produce magnetically controlled, or guided, microcapsules covered with ethylcellulose containing the antibiotic mitomycin-C (Fujimoto S. et al., Cancer, 56: 2404-2410, 1985).”

As is also disclosed in U.S. Pat. No. 4,871,716, “Another method is to produce magnetically controlled liposomes 200 nm to 800 nm in size carrying preparations that can dissolve atherosclerotic formations. This method is based on the ability of phospholipids to create closed membrane structures in the presence of water (Gregoriadis G., Ryman B. E., Biochem. J., 124:58, 1971).”

As is also disclosed in U.S. Pat. No. 4,871,716, “The above compositions require extremely high flux density magnetic fields for their control, and are somewhat difficult to produce consistently, sterilize, and store on an industrial scale without changing their designated properties.”

As is also disclosed in U.S. Pat. No. 4,871,716, “To overcome these shortcomings, a method for producing magnetically controlled dispersion has been suggested (See European Patent Office Publication No. 0 451 299 A1, by Kholodov L. E., Volkonsky V. A., Kolesnik N. F. et al.), using ferrocarbon particles as a ferromagnetic material. The ferrocarbon particles are produced by heating iron powder made up of particles 100 μm to 500 μm in size at temperatures of 800° C. to 1200° C. in an oxygen containing atmosphere. The mixture is subsequently treated by carbon monoxide at 400° C. to 700° C. until carbon particles in an amount of about 10% to 90% by mass begin emerging on the surface. A biologically active substance is then adsorbed on the particles. This method of manufacturing ferrocarbon particles is rather complicated and requires a considerable amount of energy. Because the ferromagnetic component is oxidized due to the synthesis of ferrocarbon particles at a high temperature in an oxygen containing atmosphere, magnetic susceptibility of the dispersion obtained is decreased by about one-half on the average, as compared with metallic iron. The typical upper limit of adsorption of a biologically active substance on such particles is about 2.0% to 2.5% of the mass of a ferromagnetic particle. The magnetically controlled particle produced by the above method has a spheroidal ferromagnetic component with a thread-like carbon chain extending from it and is generally about 2.0 μm in size. The structure is believed to predetermine the relatively low adsorption capacity of the composites and also leads to breaking of the fragile thread-like chains of carbon from the ferromagnetic component during storage and transportation.”

The magnetically responsive composition described in claim 7 of United States patent has paclitaxel adsorbed on its particles. A process for producing this composition is disclosed in Example 4 of the patent.

As is disclosed in such Example 4 of U.S. Pat. No. 6,200,547, “The results in Table 3 show that binding of the drug to the carrier particles is highly influenced by the composition of the adsorption solution or medium. Camptothecin is a highly non-polar molecule. In a highly non-polar adsorption medium (chloroform-ethanol), the drug does not preferentially leave the adsorption medium to adsorb to the carbon. However, in a more polar adsorption medium, it is believed that adsorption to the carrier particles would be entirely acceptable. One of the factors that influence the adsorption of the drug in the adsorption medium to the carbon in the carrier particle is the hydrophobic Van der Waals interactions between the drug and the particles. Alternatively, the drug can be dried onto the particles by evaporation techniques similar to those used for adsorption of PAC.”

As is also disclosed in U.S. Pat. No. 4,871,716, “The carrier particles used for adsorption of paclitaxel (PAC) have an iron:carbon content of 70:30. The carbon is activated carbon type E. To analytically determine the iron content the following procedure was used. A portion of the sample was weighed (previously dried in a vacuum desiccator) and washed at 1000° C., oxidizing all carbon and iron present. During this procedure carbon was converted quantitatively to CO2 and volatilized, leaving a residue of Fe2 O3. The iron content was calculated by the formula. Fe=Fe2 O3/1.42977, assuming no Fe2 O3 was present initially. Carbon was assumed to be the remaining fraction. A second analysis of another portion of the sample was performed on a LECO carbon combustion analyzer. The sample was combusted and the CO2 then measured, and total carbon was calculated. Iron and carbon content calculated by both methods gave comparable results of about 69% by weight of elemental iron. A. Binding properties of Paclitaxel to composite particles”

As is also disclosed in U.S. Pat. No. 4,871,716, “Drug adsorption was measured in two ways: 1) Initially a UV spectrophotometric assay was developed for screening drug bound to a variety of activated carbons. HPLC or spectrophotometric grade solvents were used throughout. The .lambda.max in ethanol was determined to be 220 nm. A Milton Roy Spectronic 21 spectrophotometer was used with 3 mL quartz cells. The wavelength of 254 nm was selected for UV analysis because it provided good sensitivity for the drug. Little or no contamination from various assay techniques or materials was found at that wavelength. The same wavelength was used for the HPLC analysis. The UV assay was linear for paclitaxel over the range 0.05-3.0 mg/mL.”

As is also disclosed in U.S. Pat. No. 4,871,716, “In one test the carrier particles contained the KB-type carbon. It has a small pore size (˜40 nm effective radius), >1000 m2/gm surface areas, and good hardness. PAC adsorption capacity however was limited. A survey of some 20 other candidate activated carbons was reduced to three types with promising drug delivery properties, A, B, and E types of carbon. Iron powder alone was also tested. Each of these materials was used at a concentration of 30 mg in citrated ethanol. The analysis by UV methods gave the following binding results for 3 mg of PAC. Type A carbon—74%, Type B carbon=65%, Type E carbon=33%, and iron powder=0% (no binding) Types A and B carbon are both large pore, large surface area (>=1,800 m2/gm) carbons with drug release characteristics equivalent to the E-type. E-type is a much harder carbon with a smaller surface area and consequently better milling properties. B. Paclitaxel Binding to Different Activated Carbons.”

At column 14 of U.S. Pat. No. 6,200,547, a discussion was presented of the binding affinity of paclitaxel to different types of activated carbons. It was disclosed (at lines 47 et seq.) that “fractional binding (fb) (amount bound of initial amount of PAC) to activated carbon types A, B, and E increased with increasing amount of carbon (at fixed PAC concentration). Types A and B carbon could be shown to bind PAC 100% and to plateau in the binding curve at high activated carbon content. Fractional bind of Type E was only 68%. The binding capacity, Q (expressed as % weight/weight drug carrier) was shown to decrease with an increase in the amount of activated carbon. For type A carbon, the binding capacity, Q, increased from 8% to 44% for a decrease in carbon from 40 mg to 5 mg. The corresponding Q value for AC type E was about 5% to 7%.”

As is also disclosed in U.S. Pat. No. 6,200,547, “Other studies of drug binding to type A carbon have suggested that a plateau in the fraction of drug bound as a function of the amount of absorber is a result of multilaminar drug coating on the surface of the carrier. In contrast, a linear increase in fraction bound is indicative of unilaminar coating, thus in keeping with the rules of the Langmuir isotherm analysis.”

As is also disclosed in U.S. Pat. No. 6,200,547, “Our studies showed that Types A and E carbon have the ability to adsorb a considerable fraction (fb) of PAC in the adsorption medium and that their binding capacity, Q, is also significant. On the other hand, carrier particles having a iron:carbon ratio of 70:30 (type E carbon) had both reduced capacity and fractional binding. These reduced values are in keeping with the proportionally lower carbon content of the carrier particles as compared with carbon alone. In contrast, both the fb and Q values for the carrier particles with a higher binding capacity type A carbon were less than 2%. This may be due to the inability of the pores in the carbon to withstand the compressive forces of the attrition milling process during manufacture.”

As is also disclosed in U.S. Pat. No. 4,871,716, “Despite the extensive binding of activated carbon Types A and B to PAC, use of Type E carbon in carrier particles was preferred due to commercial availability, and the proper balance between binding and release properties. In addition, Type E carbon is the preferred activated carbon for use in a drug carrier because it has been established to have U.S. Pharmacopoeia (22nd edition) quality. FIG. 6 shows Langmuir adsorption plots for PAC binding to (--.largecircle.--) carrier particles with an iron:carbon ratio of 70%:30% Type E carbon and (--.quadrature.--) Type E carbon alone. Data were fit by simple unweighted linear regression.”

As is also disclosed in U.S. Pat. No. 4,871,716, “Affinity (Km) and maximal binding (Qm) constants for PAC to the carrier particles having an iron:carbon ratio of 70:30 (Type E carbon) were determined over a range of carrier amounts. Table 4 below shows the results of adsorption isotherms of these compositions. The values were determined graphically from FIG. 6 and Langmuir's equation.”

At column 16 of U.S. Pat. No. 6,200,547, and in summarizing the results obtained in the experiments of Example 4, the patentees concluded that: “These results demonstrated that pharmacologically active paclitaxel can be released from the carrier particles of the invention, and that the chemical analysis of adsorbed and released drug can be confirmed biologically. Similar dose-response curves were obtained for free paclitaxel and paclitaxel desorbed from the carrier particles.”

One may use “ . . . pharmacogically active palitaxel . . . ” adsorbed on “ . . . the carrier particles of the invention . . . .” Thus, e.g., one may use such paclitaxel adsorbed on a composition comprised of nanomagnetic material and polymeric material material 14.

By way of further illustration, one may use the magnetically controllable ferrocarbon particle compositions of U.S. Pat. No. 6,482,436 to deliver paclitaxel to an implanted medical device; the entire disclosure of this United States patent is hereby incorporated by reference into this specification.

Claim 1 of U.S. Pat. No. 6,482,436 describes: “A magnetically responsive composition comprising particles including carbon and iron, wherein the carbon is substantially uniformly distributed throughout the particle volume, wherein the cross-sectional size of each particle is less than about 5 μm, and wherein the carbon is selected from the group consisting of types A, B, E, K, KB, and chemically modified versions thereof.”

In column 1 of U.S. Pat. No. 6,482,436, reference is made to “prior art” carrier compositions onto which a therapeutic agent is adsorbed. Thus, as is disclosed at lines 26 et seq. of column 1 of such patent, “Metallic carrier compositions used in the treatment of various disorders have been heretofore suggested and/or utilized (see, for example, U.S. Pat. Nos. 4,849,209 and 4,106,488), and have included such compositions that are guided or controlled in a body in response to external application of a magnetic field (see, for example, U.S. Pat. Nos. 4,501,726, 4,652,257 and 4,690,130). Such compositions have not always proven practical and/or entirely effective. For example, such compositions may lack adequate capacity for carriage of the desired biologically active agent to the treatment site, have less than desirable magnetic susceptibility and/or be difficult to manufacture, store and/or use.”

As is also disclosed in U.S. Pat. No. 6,482,436, “One such known composition, deliverable by way of intravascular injection, includes microspheres made up of a ferromagnetic component covered with a biocompatible polymer (albumin, gelatin, and polysaccharides) which also contains a drug (Driscol C. F. et al. Prog. Am. Assoc. Cancer Res., 1980, p. 261).”

As is also disclosed in U.S. Pat. No. 6,482,436, “It is possible to produce albumen microspheres up to 3.0 μm in size containing a magnetic material (magnetite Fe3 O4) and the anti-tumoral antibiotic doxorubicin (Widder K. et al. J. Pharm. Sci., 68:79-82 1979). Such microspheres are produced through thermal and/or chemical denaturation of albumin in an emulsion (water in oil), with the input phase containing a magnetite suspension in a medicinal solution. Similar technique has been used to produce magnetically controlled, or guided, microcapsules covered with ethylcellulose containing the antibiotic mitomycin-C (Fujimoto S. et al., Cancer, 56: 2404-2410,1985).”

U.S. Pat. No. 6,482,436 discloses that even biologically active substances that are substantially insoluble inwater can be adsorbed onto the carrier particles of this patent. As is disclosed in such column 6, commencing at line 29 thereof, “However, adsorption of biologically active substances that are substantially insoluble in water (i.e., with solubility in water less than about 0.1% by weight) requires use of special procedures to adsorb a useful amount of a drug on the particles. Applicants have discovered that adsorption on the carrier particles of this invention of biologically active substances having substantial insolubility in water can be obtained without the use of surfactants, many of which are toxic, by dissolving the water insoluble biologically active substance in a liquid adsorption medium (e.g., aqueous) that includes excipients selected to minimize the hydrophobic Van der Waals forces between the particles and the solution and to prevent agglomeration of the particles in the medium. For example, if the biologically active substance is a highly non-polar molecule, such as camptothecin, and the adsorption medium is a highly non-polar liquid, such as chloroform-ethanol, the drug does not preferentially leave the adsorption medium to adsorb to the carbon. However, in a more polar adsorption medium, adsorption to the carrier particles is entirely acceptable. For example, binding of therapeutic levels of paclitaxel, a highly water-insoluble drug, to carrier particles having an iron:carbon ratio of 70:30 was obtained using citrated ethanol as the adsorption medium, even though paclitaxel is substantially water insoluble. In many cases, it is advantageous if the liquid adsorption medium includes a biologically compatible and biodegradable viscosity-increasing agent (e.g., a biologically compatible polymer), such as sodium carboxymethyl cellulose, to aid in separation of the particles in the medium.”

In Example 5 of U.S. Pat. No. 6,482,436, (see column 15), an experiment was described in which paclitaxel was absorbed onto carrier particles having an iron/carbon ratio of 70/30. As was disclosed insuch column 15, “The carrier particles used for adsorption of paclitaxel (PAC) have an iron:carbon content of 70:30. The carbon is activated carbon type E. To analytically determine the iron content the following procedure was used. A portion of the sample was weighed (previously dried in a vacuum desiccator) and washed at 2000° C., oxidizing all carbon and iron present. During this procedure carbon was converted quantitatively to CO2 and volatilized, leaving a residue of Fe2 O3. The iron content was calculated by the formula. Fe=Fe2 O3/1.42977, assuming no Fe2 O3 was present initially. Carbon was assumed to be the remaining fraction. A second analysis of another portion of the sample was performed on a LECO carbon combustion analyzer. The sample was combusted and the CO2 then measured, and total carbon was calculated. Iron and carbon content calculated by both methods gave comparable results of about 69% by weight of elemental iron.”

The Use of Externally Applied Energy to Affect an Implanted Medical Device

The prior art discloses many devices in which an externally applied electromagnetic field (i.e., a field originating outside of a biological organism, such as a human body) is generated in order to influence one or more implantable devices disposed within the biological organism. Some of these devices are described below; they may be used in the processes and apparatuses of the instant invention (see, e.g., radiation source 41 of FIG. 1A).

U.S. Pat. No. 3,337,776 describes a device for producing controllable low frequency magnetic fields; the entire disclosure of this patent is hereby incorporated by reference into this specification. Thus, e.g., claim 1 of this patent describes a biomedical apparatus for the treatement of a subject with controllable low frequency magnetic fields, comprising solenoid mens for creating the magnetic field.

U.S. Pat. No. 3,890,953 also discloses an apparatus for promoting the growth of bone and other body tissues by the application of a low frequency alternating magnetic field; the entire disclosure of this United States patent is hereby incorporated by reference into this specification. This patent claims “In an electrical apparatus for promoting the growth of bone and other body tissues by the application thereto of a low frequency alternating magnetic field, such apparatus having current generating means and field applicator means, the improvement wherein the applicator means comprises a flat solenoid coil having an axis about which the coil is wound and composed of a plurality of parallel and flexible windings, each said winding having two adjacent elongate portions and two 180° coil bends joining said elongate portions together, said coil being flexible in the coil plane in the region of said elongate portion for being bent into a U-shape, said coil being bent into such U-shape about an axis parallel to the coil axis and adapted for connection to a source of low frequency alternating current.”

The device of U.S. Pat. No. 3,890,953 is described, in part, at lines 52 et seq. of column 2, wherein it is disclosed that: “.The apparatus shown diagrammatically in FIG. 1 comprises a AC generator 10, which supplies low frequency AC at the output terminals 12. The frequency of the AC lies below 150 Hz, for instance between 1 and 50 or 65 Hz. It has been found particularly favorable to use a frequency range between 5 or 10 and 30 Hz, for example 25 Hz. The half cycles of the alternating current should have comparatively gently sloping leading and trailing flanks (rise and fall times of the half cycles being for example in the order of magnitude of a quarter to an eighth of the length of a cycle); the AC can thus be a sinusoidal current with a low non-linear distortion, for example less than 20 percent, or preferably less than 10 percent, or a triangular wave current.”

U.S. Pat. No. 4,095,588 discloses a “vascular cleansing device” adapted to “ . . . effect motion of thered corpuscles in the blood stream of a vascular system . . . wherey these red cells may cleanse the vascular system by scrubbing the walls thereof . . . ;” the entire disclosure of this United States patent is hereby incorporated by reference into this specification. This patent claims (in claim 3) “A means to propel a red corpuscle in a vibratory and rotary fashion, said means comprising an electronic circuit and magnetic means including: a source of electrical energy; a variable oscillator connected to said source; a binary counter means connected to said oscillator to produce sequential outputs; a plurality of deflection amplifier means connected to be operable by the outputs of said binary counter means in a sequential manner, said amplifier means thereby controlling electrical energy from said source; a plurality of separate coils connected in separate pairs about an axis in series between said deflection amplifier means and said source so as to besequentially operated in creating an electromagnetic field from one coil to the other and back again and thence to adjacent separate coils for rotation of the electromagnetic field from one pair of coils to another; and a table within the space encircled by said plurality of coils, said table being located so as to place a person along the axis such that the red corpuscles of the person's vascular system are within the electromagnetic field between the coils creating same.”

U.S. Pat. No. 4,323,075 discloses an implantable defibrillator with a rechargeable power supply; the entire disclosure of this patent is herebyh incorporated by reference into this specification. Claim 1 of this patent describes “A fully implantable power supply for use in a fully implantable defibrillator having an implantable housing, a fibrillation detector for detecting fibrillation of the heart of a recipient, an energy storage and discharge device for storing and releasing defibrillation energy into the heart of the recipient and an inverter for charging the energy storage and discharge device in response to detection of fibrillation by the fibrillation detector, the inverter requiring a first level of power to be operational and the fibrillation detector requiring a second level of power different from said first level of power to be operational, said power supply comprising: implantable battery means positioned within said implantable housing, said battery means including a plurality of batteries arranged in series, each of said batteries having a pair of output terminals, each of said batteries producing a distinctly multilevel voltage across its pair of output terminals, said voltage being at a first level when the battery is fully charged and dropping to a second level at some point during the discharge of the battery; and implantable circuit means positioned within said implantable housing, said circuit means for creating a first conductive path betwen said serially-connected batteries and said fibrillation detector to provide said fibrillation detector with said second level of power, and for creating a second conductive path between said inverter and said battery means by placing only the batteries operating at said first level voltage in said second conductive path, and excluding the remaining batteries from said second conductive path to provide said inverter with said first level of power.”

U.S. Pat. No. 4,340,038 discloses an implanted medical system comprised of magnetic field pick-up means for converting magnetic energy to electrical energy; the entire disclosure of this patentis hereby incorporated by reference into this specification.

In column 1 of U.S. Pat. No. 4,340,038, at lines 12 et seq., it is disclosed that “Many types of implantable devices incorporate a self-contained transducer for converting magnetic energy from an externally-located magnetic field generator to energy usable by the implanted device. In such a system having an implanted device and an externally-located magnetic field generator for powering the device, sizing and design of the power transfer system is important. In order to properly design the power transfer system while at the same time avoiding overdesign, the distance from the implanted device to the magnetic field generator must be known. However for some types of implanted devices the depth of the implanted device in a recipient's body is variable, and is not known until the time of implantation by a surgeon. One example of such a device is an intracranial pressure monitoring device (ICPM) wherein skull thickness varies considerably between recipients and the device must be located so that it protrudes slightly below the inner surface of the skull and contacts the dura, thereby resulting in a variable distance between the top of the implanted device containing a pick-up coil or transducer and the outer surface of the skull. One conventional technique for accommodating an unknown distance between the magnetic field generator and the implanted device includes increasing the transmission power of the external magnetic field generator. However this increased power can result in heating of the implanted device, the excess heat being potentially hazardous to the recipient. A further technique has been to increase the diameter of the pick-up coil in the implanted device. However, physical size constraints imposed on many implanted devices such as the ICPM are critical; and increasing the diameter of the pick-up coil is undesirable in that it increases the size of the orifice which must be formed in the recipient's skull. The concentrator of the present invention solves the above problems by concentrating magnetic lines of flux from the magnetic generator at the implanted pick-up coil, the concentrator being adapted to accommodate distance variations between the implanted device and the magnetic field generator.”

Claim 1 of U.S. Pat. No. 4,340,038 describes “In a system including an implanted device having a magnetic field pick-up means for converting magnetic energy to electrical energy for energizing said implanted device, and an external magnetic field generator located so that magnetic lines of flux generated thereby intersect said pick-up means, a means for concentrating a portion of said magnetic lines of flux at said pick-up means comprising a metallic slug located between said generator and said pick-up means, thereby concentrating said magnetic lines of flux at said pick-up means.” Claim 5 of this patent further describes the pick-up means as comprising “ . . . a magnetic pick-up coil and said slug is formed in the shape of a truncated cone and oriented so that a plane defined by the smaller of said cone end surfaces is adjacent to said substantially parallel to a plane defined by said magnetic pick-up coil.”

U.S. Pat. No. 4,361,153 discloses an implantable telemetry system; the entire disclosure of such United States patent is hereby incorporated by reference into this specification.

As is disclosed at column I of U.S. Pat. No. 4,361,153 (see lines 9 et seq.), “Externally applied oscillating magnetic fields have been used before with implanted devices. Early inductive cardiac pacers employed externally generated electromagnetic energy directly as a power source. A coil inside the implant operated as a secondary transformer winding and was interconnected with the stimulating electrodes. More recently, implanted stimulators with rechargeable (e.g., nickel cadmium) batteries have used magnetic transmission to couple energy into a secondary winding in the implant to energize a recharging circuit having suitable rectifier circuitry. Miniature reed switches have been utilized before for implant communications. They appear to have been first used to allow the patient to convert from standby or demand mode to fixed rate pacing with an external magnet. Later, with the advent of programmable stimulators, reed switches were rapidly cycled by magnetic pulse transmission to operate pulse parameter selection circuitry inside the implant. Systems analogous to conventional two-way radio frequency (RF) and optical communication system have also been proposed. The increasing versatility of implanted stimulators demands more complex programming capabilities. While various systems for transmitting data into the implant have been proposed, there is a parallel need to develop compatible telemetry systems-for signalling out of the implant. However, the austere energy budget constraints imposed by long life, battery operated implants rule out conventional transmitters and analogous systems”

The solution provided by U.S. Pat. No. 4,361,153 is “ . . . achieved by the use of a resonant impedance modulated transponder in the implant to modulate thephase of a relatively high energy reflected magnetic carrier imposed from outside of the body.” In particular, and as is described by claim 1 of this patent, there is claimed “An apparatus for communicating variable information to an external device from an electronic stimulator implanted in a living human patient, comprising an external unit including means for transmitting a carrier signal, a hermetically sealed fully implantable enclosure adapted to be implanted at a fixed location in the patient's body, means within said enclosure for generating stimulator outputs, a transponder within said enclosure including tuned resonant circuit means for resonating at the frequency of said carrier signal so as to re-radiate a signal at the frequency of said carrier signal, and means for superimposing an information signal on the reflected signal by altering the resonance of said tuned circuit means in accordance with an information signal, said superimposing means including a variable impedance load connected across said tuned circuit and means for varying the impedance of said load in accordance with an information signal, said external unit further including pickup means for receiving the reflected signal from said transponder and means for recovering the information signal superimposed thereon, said receiving means including means reponsive to said reflected signal from said transponder for producing on associated analog output signal, and said recovering means including phase shift detector means responsive to said analog output signal for producing an output signal related to the relative phase angle thereof.”

U.S. Pat. No. 4,408,607 discloses a rechargeable, implantable capacitive energy source; the entire disclosure of this patent is hereby incorporated into this specification by reference. As is disclosed in column 1 of such patent (at lines 12 et seq.), “Medical science has advanced to the point where it is possible to implant directly within living bodies electrical devices necessary or advantageous to the welfare of individual patients. A problem with such devices is how to supply the electrical energy necessary for their continued operation. The devices are, of course, designed to require a minimum of electrical energy, so that extended operation from batteries may be possible. Lithium batteries and other primary, non-rechargeable cells may be used, but they are expensive and require replacement of surgical procedures. Nickel-cadmium and other rechargeable batteries are also available, but have limited charge-recharge characteristics, require long intervals for recharging, and release gas during the charging process.”

The solution to this problem is described, e.g., in claim 1 of U.S. Pat. No. 4,408,607, which describes “An electric power supply for providing electrical energy to an electrically operated medical device comprising: capacitor means for accommodating an electric charge; first means providing a regulated source of unidirectional electrical energy; second means connecting said first means to said capacitor means for supplying charging current to said capacitor means at a first voltage which increases with charge in the capacitor means; third means deriving from said first means a comparison second voltage of constant magnitude; comparator means operative when said first voltage reaches a first value to reduce said first voltage to a second, lower value; and voltage regulator means connected to said capacitor means and medical device to limit the voltage supplied to the medical device.”

U.S. Pat. No. 4,416,283 discloses a implantable shunted coil telemetry transponder employed as a magnetic pulse transducer for receiving externally transmitted data; the entire disclosure of this United States patent is hereby incorporated by reference into this specification.

In particular, a programming system for a biomedical implant is described in claim 1 of U.S. Pat. No. 4,416,283. Such claim 1 discloses “In a programming system for a biomedical implant of the type wherein an external programmer produces a series of magnetic impulses which are received and transduced to form a corresponding electrical pulse input to programmable parameter data registers inside the implant, wherein the improvement comprises external programming pulse receiving and transducing circuitry in the implant including a tuned coil, means responsive to pairs of successive voltage spikes of opposite polarity magnetically induced across said tuned coil by said magnetic impulses for forming corresponding binary pulses duplicating said externally generated magnetic impulses giving rise to said spikes, and means for outputting said binary pulses to said data registers to accomplish programming of the implant.”

U.S. Pat. No. 4,871,351 discloses an implantale pump infusion system; the entire disclosure of this United States patent is hereby incorporated by reference into this specification. These implantable pumps are disussed in column 1 of the patent, wherein it is disclosed that: “Certain human disorders, such as diabetes, require the injection into the body of prescribed amounts of medication at prescribed times or in response to particular conditions or events. Various kinds of infusion pumps have been propounded for infusing drugs or other chemicals or solutions into the body at continuous rates or measured dosages. Examples of such known infusion pumps and dispensing devices are found in U.S. Pat. Nos. 3,731,861; 3,692,027; 3,923,060; 4,003,379; 3,951,147; 4,193,397; 4,221,219 and 4,258,711. Some of the known pumps are external and inject the drugs or other medication into the body via a catheter, but the preferred pumps are those which are fully implantable in the human body.”

As is disclosed in U.S. Pat. No. 4,871,351, “Implantable pumps have been used in infusion systems such as those disclosed in U.S. Pat. Nos. 4,077,405; 4,282,872; 4;270,532; 4,360,019 and 4,373,527. Such infusion systems are of the open loop type. That is, the systems are pre-programmed to deliver a desired rate of infusion. The rate of infusion may be programmed to vary with time and the particular patient. A major disadvantage of such open loop systems is that they are not responsive to the current condition of the patient, i.e. they do not have feedback information. Thus, an infusion system of the open loop type may continue dispensing medication according to its pre-programmed rate or profile when, in fact, it may not be needed.”

As is also disclosed in U.S. Pat. No. 4,871,351, “There are known closed loop infusion systems which are designed to control a particular condition of the body, e.g. the blood glucose concentration. Such systems use feedback control continuously, i.e. the patient's blood is withdrawn via an intravenous catheter and analysed continuously and a computer output signal is derived from the actual blood glucose concentration to drive a pump which infuses insulin at a rate corresponding to the signal. The known closed loop systems suffer from several disadvantages. First, since they monitor the blood glucose concentration continuously they are complex and relatively bulky systems external to the patient, and restrict the movement of the patient. Such systems are suitable only for hospital bedside applications for short periods of time and require highly trained operating staff. Further, some of the known closed loop systems do not allow for manually input overriding commands. Examples of closed loop systems are found in U.S. Pat. Nos. 4,055,175; 4,151,845 and 4,245,634.”

As is also disclosed in U.S. Pat. No. 4,871,351, “An implanted closed loop system with some degree of external control is disclosed in U.S. Pat. No. 4,146,029. In that system, a sensor (either implanted or external) is arranged on the body to sense some kind of physiological, chemical, electrical or other condition at a particular site and produced data which corresponds to the sensed condition at the sensed site. This data is fed directly to an implanted microprocessor controlled medication dispensing device. A predetermined amount of medication is dispensed in response to the sensed condition according to a pre-programmed algorithm in the microprocessor control unit. An extra-corporeal coding pulse transmitter is provided for selecting between different algorithms in the microprocessor control unit. The system of U.S. Pat. No. 4,146,029 is suitable for use in treating only certain ailments such as cardiac conditions. It is unsuitable as a blood glucose control system for example, since (i) it is not practicable to measure the blood glucose concentration continuously with an implanted sensor and (ii) the known system is incapable of dispensing discrete doses of insulin in response to certain events, such as meals and exercise. Furthermore, there are several disadvantages to internal sensors; namely, due to drift, lack of regular calibration and limited life, internal sensors do not have high long-term reliability. If an external sensor is used with the system of U.S. Pat. No. 4,146,029, the output of the sensor must be fed through the patient's skin to the implanted mechanism. There are inherent disadvantages to such a system, namely the high risk of infection. Since the algorithms which control the rate of infusion are programmed into the implanted unit, it is not possible to upgrade these algorithms without surgery. The extra-corporeal controller merely selects a particular one of several medication programs but cannot actually alter a program.”

As is also disclosed in U.S. Pat. No. 4,871,351, “It is an object of the present invention to overcome, or substantially ameliorate the above described disadvantages of the prior art by providing an implantable open loop medication infusion system with a feedback control option”

The solution to this problem is set forth in claim 1 of U.S. Pat. No. 4,871,351, which describes: “A medical infusion system intermittently switchable at selected times between an open loop system without feedback and a closed loop system with feedback, said system comprising an implantable unit including means for controllably dispensing medication into a body, an external controller, and an extra-corporeal sensor; wherein said implantable unit comprises an implantable transceiver means for communicating with a similar external transceiver means in said external controller to provide a telemetry link between said controller and said implantable unit, a first reservoir means for holding medication liquid, a liquid dispensing device, a pump connected between said reservoir means and said liquid dispensing device, and a first electronic control circuit means connected to said implantable transceiver means and to said pump to operate said pump; wherein said external controller comprises a second electronic control circuit means connected with said external transceiver means, a transducer means for reading said sensor, said transducer means having an output connected to said second electronic control circuit means, and a manually operable electric input device connected to said second electronic control circuit means; wherein said pump is operable by said first electonic control circuit means to pump said medication liquid from said first reservoir means to said liquid-dispensing deive at a first predetermined rate independent of the output of said extra-corporeal sensor, and wherein said input device or said transducer means include means which selectively operable at intermittent times to respectively convey commands or output of said transducer representing the reading of said sensor to said second control circuit to instruct said first control circuit via said telemetry link to modify the operation of said pump.”

U.S. Pat. No. 4,941,461 describes an electrically actuated inflatable penile erecton device comprised of an implantable induction coil and an implantable pump; the entire disclosure of this United States patent is hereby incorporated by reference into this specification. The device of this patent is described, e.g., in claim 1 of the patent, which discloses “An apparatus for achieving a penile erection in a human male, comprising: at least one elastomer cylinder having a root chamber and a pendulous chamber, said elastomer cylinder adapted to be placed in the corpus carvenosum of the penis; an external magnetic field generator which can be placed over some section of the penis which generates an alternating magnetic field; an induction coil contained within said elastomer cylinder which produces an alternating electric current when in the proximity of said alternating magnetic filed which is produced by said external magnetic field generator; and a fluid pumping means located within said elastomer cylinder, said pumping means being operated by the electrical power generated in said induction coil to pump fluid from said root chamber to said pendulous chamber in order to stiffen said elastomer cylinder for causing the erect state of the penis.”

U.S. Pat. No. 5,487,760 discloses an implantable signal transceiver disposed in an artificial heart valve; the entire disclosure of this United States patent is hereby incorporated by reference into this specification. Claim 1 of this patent describes: “In combination, an artificial heart valve of the type having a tubular body member, defining a lumen and pivotally supporting at least one occluder, said body member having a sewing cuff covering an exterior surface of said body member; and an electronic sensor module disposed between said sewing cuff and said exterior surface, wherein said sensor module incorporates a sensor element for detecting movement of said at least one occluder between an open and a closed disposition relative to said lumen and wherein said sensor module further includes a signal transceiver coupled to said sensor element, and means for energizing said signal transceiver, and wherein said sensor module includes means for encapsulating said sensor element, signal transceiver and energizing means in a moisture-impervious container.”

U.S. Pat. No. 5,702,430 discloses an implantable power supply; the entire disclosure of such patent is hereby incorporated by reference into this specification. Claim 1 of such patent describes: “A surgically implantable power supply comprising battery means for providing a source of power, charging means for charging the battery means, enclosure means isolating the battery means from the human body, gas holding means within the enclosure means for holding gas generated by the battery means during charging, seal means in the enclosure means arranged to rapture when the internal gas pressure exceeds a certain value and inflatable gas container means outside the enclosure means to receive gas from within the enclosure means when the seal means has been ruptured.”

Columns 1 through 5 of U.S. Pat. No. 5,702,430 presents an excellent discussion of “prior art” implantable pump assemblies. As is disclosed in such portion of U.S. Pat. No. 5,702,430, “The most widely tested and commonly used implantable blood pumps employ variable forms of flexible sacks (also spelled sacs) or diaphragms which are squeezed and released in a cyclical manner to cause pulsatile ejection of blood. Such pumps are discussed in books or articles such as Hogness and Antwerp 1991, DeVries et al 1984, and Farrar et al 1988, and in U.S. Pat. No. 4,994,078 (Jarvik 1991), U.S. Pat. No. 4,704,120 (Slonina 1987), U.S. Pat. No. 4,936,758 (Coble 1990), and U.S. Pat. No. 4,969,864 (Schwarzmann et al 1990). Sack or diaphragm pumps are subject to fatigue failure of compliant elements and as such are mechanically and functionally quite different from the pump which is the subject of the present invention.”

As is also disclosed in U.S. Pat. No. 5,702,430, “An entirely different class of implantable blood pumps uses rotary pumping mechanisms. Most rotary pumps can be classified into two categories: centrifugal pumps and axial pumps. Centrifugal pumps, which include pumps marketed by Sarns (a subsidiary of the 3M Company) and Biomedicus (a subsidiary of Medtronic, Eden Prairie, Minn.), direct blood into a chamber, against a spinning interior wall (which is a smooth disk in the Medtronic pump). A flow channel is provided so that the centrifugal force exerted on the blood generates flow.”

As is also disclosed in U.S. Pat. No. 5,702,430, “By contrast, axial pumps provide blood flow along a cylindrical axis, which is in a straight (or nearly straight) line with the direction of the inflow and outflow. Depending on the pumping mechanism used inside an axial pump, this can in some cases reduce the shearing effects of the rapid acceleration and deceleration forces generated in centrifugal pumps. However, the mechanisms used by axial pumps can inflict other types of stress and damage on blood cells.”

As is also disclosed in U.S. Pat. No. 5,702,430, “Some types of axial rotary pumps use impeller blades mounted on a center axle, which is mounted inside a tubular conduit. As the blade assembly spins, it functions like a fan, or an outboard motor propeller. As used herein, “impeller” refers to angled vanes (also called blades) which are constrained inside a flow conduit; an impeller imparts force to a fluid that flows through the conduit which encloses the impeller. By contrast, “propeller” usually refers to non-enclosed devices, which typically are used to propel vehicles such as boats or airplanes.”

As is also disclosed in U.S. Pat. No. 5,702,430, “Another type of axial blood pump, called the “Haemopump” (sold by Nimbus) uses a screw-type impeller with a classic screw (also called an Archimedes screw; also called a helifoil, due to its helical shape and thin cross-section). Instead of using several relatively small vanes, the Haemopump screw-type impeller contains a single elongated helix, comparable to an auger used for drilling or digging holes. In screw-type axial pumps, the screw spins at very high speed (up to about 10,000 rpm). The entire Haemopump unit is usually less than a centimeter in diameter. The pump can be passed through a peripheral artery into the aorta, through the aortic valve, and into the left ventricle. It is powered by an external motor and drive unit.”

As is also disclosed in U.S. Pat. No. 5,702,430, “Centrifugal or axial pumps are commonly used in three situations: (1) for brief support during cardio-pulmonary operations, (2) for short-term support while awaiting recovery of the heart from surgery, or (3) as a bridge to keep a patient alive while awaiting heart transplantation. However, rotary pumps generally are not well tolerated for any prolonged period. Patients who must rely on these units for a substantial length of time often suffer from strokes, renal (kidney) failure, and other organ dysfunction. This is due to the fact that rotary devices, which must operate at relatively high speeds, may impose unacceptably high levels of turbulent and laminar shear forces on blood cells. These forces can damage or lyse (break apart) red blood cells. A low blood count (anemia) may result, and the disgorged contents of lysed blood cells (which include large quantities of hemoglobin) can cause renal failure and lead to platelet activation that can cause embolisms and stroke.”

As is also disclosed in U.S. Pat. No. 5,702,430, “One of the most important problems in axial rotary pumps in the prior art involves the gaps that exist between the outer edges of the blades, and the walls of the flow conduit. These gaps are the site of severe turbulence and shear stresses, due to two factors. Since implantable axial pumps operate at very high speed, the outer edges of the blades move extremely fast and generate high levels of shear and turbulence. In addition, the gap between the blades and the wall is usually kept as small as possible to increase pumping efficiency and to reduce the number of cells that become entrained in the gap area. This can lead to high-speed compression of blood cells as they are caught in a narrow gap between the stationary interior wall of the conduit and the rapidly moving tips or edges of the blades.”

As is also disclosed in U.S. Pat. No. 5,702,430, “An important factor that needs to be considered in the design and use of implantable blood pumps is “residual cardiac function,” which is present in the overwhelming majority of patients who would be candidates for mechanical circulatory assistance. The patient's heart is still present and still beating, even though, in patients who need mechanical pumping assistance, its output is not adequate for the patient's needs. In many patients, residual cardiac functioning often approaches the level of adequacy required to support the body, as evidenced by the fact that the patient is still alive when implantation of an artificial pump must be considered and decided. If cardiac function drops to a level of severe inadequacy, death quickly becomes imminent, and the need for immediate intervention to avert death becomes acute.”

As is also disclosed in U.S. Pat. No. 5,702,430, “Most conventional ventricular assist devices are designed to assume complete circulatory responsibilities for the ventricle they are “assisting.” As such, there is no need, nor presumably any advantage, for the device to interact in harmony with the assisted ventricle. Typically, these devices utilize a “fill-to-empty” mode that, for the most part, results in emptying of the device in random association with native heart contraction. This type of interaction between the device and assisted ventricle ignores the fact that the overwhelming majority of patients who would be candidates for mechanical assistance have at least some significant residual cardiac function.”

As is also disclosed in U.S. Pat. No. 5,702,430, “It is preferable to allow the natural heart, no matter how badly damaged or diseased it may be, to continue contributing to the required cardiac output whenever possible so that ventricular hemodynamics are disturbed as little as possible. This points away from the use of total cardiac replacements and suggests the use of “assist” devices whenever possible. However, the use of assist devices also poses a very difficult problem: in patients suffering from severe heart disease, temporary or intermittent crises often require artificial pumps to provide “bridging” support which is sufficient to entirely replace ventricular pumping capacity for limited periods of time, such as in the hours or days following a heart attack or cardiac arrest, or during periods of severe tachycardia or fibrillation.”

As is also disclosed in U.S. Pat. No. 5,702,430, “Accordingly, an important goal during development of the described method of pump implantation and use and of the surgically implantable reciprocating pump was to design a method and a device which could cover a wide spectrum of requirements by providing two different and distinct functions. First, an ideal cardiac pumping device should be able to provide “total” or “complete” pumping support which can keep the patient alive for brief or even prolonged periods, if the patient's heart suffers from a period of total failure or severe inadequacy. Second, in addition to being able to provide total pumping support for the body during brief periods, the pump should also be able to provide a limited “assist” function. It should be able to interact with a beating heart in a cooperative manner, with minimal disruption of the blood flow generated by the natural heartbeat. If a ventricle is still functional and able to contribute to cardiac output, as is the case in the overwhelming majority of clinical applications, then the pump will assist or augment the residual cardiac output. This allows it to take advantage of the natural, non-hemolytic pumping action of the heart to the fullest extent possible; it minimizes red blood cell lysis, it reduces mechanical stress on the pump, and it allows longer pump life and longer battery life.”

As is also disclosed in U.S. Pat. No. 5,702,430, “Several types of surgically implantable blood pumps containing a piston-like member have been developed to provide a mechanical device for augmenting or even totally replacing the blood pumping action of a damaged or diseased mammalian heart.”

As is also disclosed in U.S. Pat. No. 5,702,430, “U.S. Pat. No. 3,842,440 to Karlson discloses an implantable linear motor prosthetic heart and control system containing a pump having a piston-like member which is reciprocal within a magnetic field. The piston-like member includes a compressible chamber in the prosthetic heart which communicates with the vein or aorta.”

As is also disclosed in U.S. Pat. No. 5,702,430, “U.S. Pat. Nos. 3,911,897 and 3,911,898 to Leachman, Jr. disclose heart assist devices controlled in the normal mode of operation to copulsate and counterpulsate with the heart, respectively, and produce a blood flow waveform corresponding to the blood flow waveform of the heart being assisted. The heart assist device is a pump connected serially between the discharge of a heart ventricle and the vascular system. The pump may be connected to the aorta between the left ventricle discharge immediately adjacent the aortic valve and a ligation in the aorta a short distance from the discharge. This pump has coaxially aligned cylindrical inlet and discharge pumping chambers of the same diameter and a reciprocating piston in one chamber fixedly connected with a reciprocating piston of the other chamber. The piston pump further includes a passageway leading between the inlet and discharge chambers and a check valve in the passageway preventing flow from the discharge chamber into the inlet chamber. There is no flow through the movable element of the piston.”

As is also disclosed in U.S. Pat. No. 5,702,430, “U.S. Pat. No. 4,102,610 to Taboada et al. discloses a magnetically operated constant volume reciprocating pump which can be used as a surgically implantable heart pump or assist. The reciprocating member is a piston carrying a tilting-disk type check valve positioned in a cylinder. While a tilting disk valve results in less turbulence and applied shear to surrounding fluid than a squeezed flexible sack or rotating impeller, the shear applied may still be sufficiently excessive so as to cause damage to red blood cells.”

As is also disclosed in U.S. Pat. No. 5,702,430, “U.S. Pat. Nos. 4,210,409 and 4,375,941 to Child disclose a pump used to assist pumping action of the heart having a piston movable in a cylindrical casing in response to magnetic forces. A tilting-disk type check valve carried by the piston provides for flow of fluid into the cylindrical casing and restricts reverse flow. A plurality of longitudinal vanes integral with the inner wall of the cylindrical casing allow for limited reverse movement of blood around the piston which may result in compression and additional shearing of red blood cells. A second fixed valve is present in the inlet of the valve to prevent reversal of flow during piston reversal.”

As is also disclosed in U.S. Pat. No. 5,702,430, “U.S. Pat. No. 4,965,864 to Roth discloses a linear motor using multiple coils and a reciprocating element containing permanent magnets which is driven by microprocessor-controlled power semiconductors. A plurality of permanent magnets is mounted on the reciprocating member. This design does not provide for self-synchronization of the linear motor in the event the stroke of the linear motor is greater than twice the pole pitch on the reciprocating element. During start-up of the motor, or if magnetic coupling is lost, the reciprocating element may slip from its synchronous position by any multiple of two times the pole pitch. As a result, a sensing arrangement must be included in the design to detect the position of the piston so that the controller will not drive it into one end of the closed cylinder. In addition, this design having equal pole pitch and slot pitch results in a “jumpy” motion of the reciprocating element along its stroke.”

As is also disclosed in U.S. Pat. No. 5,702,430, “In addition to the piston position sensing arrangement discussed above, the Roth design may also include a temperature sensor and a pressure sensor as well as control circuitry responsive to the sensors to produce the intended piston motion. For applications such as implantable blood pumps where replacement of failed or malfunctioning sensors requires open heart surgery, it is unacceptable to have a linear motor drive and controller that relies on any such sensors. In addition, the Roth controller circuit uses only NPN transistors thereby restricting current flow to the motor windings to one direction only.”

As is also disclosed in U.S. Pat. No. 5,702,430, “U.S. Pat. No. 4,541,787 to Delong describes a pump configuration wherein a piston containing a permanent magnet is driven in a reciprocating fashion along the length of a cylinder by energizing a sequence of coils positioned around the outside of the cylinder. However, the coil and control system configurations disclosed only allow current to flow through one individual winding at a time. This does not make effective use of the magnetic flux produced by each pole of the magnet in the piston. To maximize force applied to the piston in a given direction, current must flow in one direction in the coils surrounding the vicinity of the north pole of the permanent magnet while current flows in the opposite direction in the coils surrounding the vicinity of the south pole of the permanent magnet. Further, during starting of the pump disclosed by Delong, if the magnetic piston is not in the vicinity of the first coil energized, the sequence of coils that are subsequently energized will ultimately approach and repel the magnetic piston toward one end of the closed cylinder. Consequently, the piston must be driven into the end of the closed cylinder before the magnetic poles created by the external coils can become coupled with the poles of the magnetic piston in attraction.”

As is also disclosed in U.S. Pat. No. 5,702,430, “U.S. Pat. No. 4,610,658 to Buchwald et al. discloses an implantable fluid displacement peritoneovenous shunt system. The system comprises a magnetically driven pump having a spool piston fitted with a disc flap valve.”

As is also disclosed in U.S. Pat. No. 5,702,430, “U.S. Pat. No. 5,089,017 to Young et al. discloses a drive system for artificial hearts and left ventricular assist devices comprising one or more implantable pumps driven by external electromagnets. The pump utilizes working fluid, such as sulfur hexafluoride to apply pneumatic pressure to increase blood pressure and flow rate.”

U.S. Pat. No. 5,743,854 discloses a device for inducing and localizing epileptiform activity that is comprised of a direct current (DC) magnetic field generator, a DC power source, and sensors adapted to be coupled to a patient's head; the entire disclosur of this United States patent is hereby incorporated by reference into this specification. In one embodiment of the invention, described in claim 7, the sensors “ . . . comprise Foramen Ovale electrodes adapted to be implanted to sense evoked and natural epileptic firings.”

U.S. Pat. No. 5,803,897 discloses a penile prosthesis system comprised of an implantable pressurized chamber, a reservoir, a rotary pump, a magnetically responsive rotor, and a rotary magnetic field generator; the entired disclosure of this United States patent is hereby incorporated by reference into this specification. Claim 1 of this patent describes: “A penile prosthesis system comprising: at least one pressurizable chamber including a fluid port, said chamber adapted to be located within the penis of a patient for tending to make the penis rigid in response to fluid pressure within said chamber; a fluid reservoir; a rotary pump adapted to be implanted within the body of a user, said rotary pump being coupled to said reservoir and to said chamber, said rotary pump including a magnetically responsive rotor adapted for rotation in the presence of a rotating magnetic field, and an impeller for tending to pump fluid at least from said reservoir to said chamber under the impetus of fluid pressure, to thereby pressurize said chamber in response to operation of said pump; and a rotary magnetic field generator for generating a rotating magnetic field, for, when placed adjacent to the skin of said user at a location near said rotary pump, rotating said magnetically responsive rotor in response to said rotating magnetic field, to thereby tend to pressurize said chamber and to render the penis rigid; controllable valve means operable in response to motion of said rotor of said rotary pump, for tending to prevent depressurization of said chamber when said rotating magnetic field no longer acts on said rotor, said controllable valve means comprising a unidirectional check valve located in the fluid path extending between said rotary pump and said port of said chamber.”

U.S. Pat. No. 5,810,015 describes an implantable power supply that can convert non-electrical energy (such as mechanical, chemical, thermal, or nuclear energy) into electrical energy; the entire disclosure of this United States patent is hereby incorporated by reference into this specification.

In column 1 of U.S. Pat. No. 5,810,015, a discussion of “prior art” rechargeable power supplies is presented. It is disclosed in this column 1 that: “Modern medical science employs numerous electrically powered devices which are implanted in a living body. For example, such devices may be employed to deliver medications, to support blood circulation as in a cardiac pacemaker or artificial heart, and the like. Many implantable devices contain batteries which may be rechargeable by transcutaneous induction of electromagnetic fields in implanted coils connected to the batteries. Transcutaneous inductive recharging of batteries in implanted devices is disclosed for example in U.S. Pat. Nos. 3,923,060; 4,082,097; 4,143,661; 4,665,896; 5,279,292; 5,314,453; 5,372,605, and many others.”

As is also disclosed in U.S. Pat. No. 5,810,015, “Other methods for recharging implanted batteries have also been attempted. For example, U.S. Pat. No. 4,432,363 discloses use of light or heat to power a solar battery within an implanted device. U.S. Pat. No. 4,661,107 discloses recharging of a pacemaker battery using mechanical energy created by motion of an implanted heart valve.”

As is also disclosed in U.S. Pat. No. 5,810,015, “A number of implanted devices have been powered without batteries. U.S. Pat. Nos. 3,486,506 and 3,554,199 disclose generation of electric pulses in an implanted device by movement of a rotor in response to the patient's heartbeat. U.S. Pat. No. 3,563,245 discloses a miniaturized power supply unit which employs mechanical energy of heart muscle contractions to generate electrical energy for a pacemaker. U.S. Pat. No. 3,456,134 discloses a piezoelectric converter for electronic implants in which a piezoelectric crystal is in the form of a weighted cantilever beam capable of responding to body movement to generate electric pulses. U.S. Pat. No. 3,659,615 also discloses a piezoelectric converter which reacts to muscular movement in the area of implantation. U.S. Pat. No. 4,453,537 discloses a pressure actuated artificial heart powered by a second implanted device attached to a body muscle which in turn is stimulated by an electric signal generated by a pacemaker.”

As is also disclosed in U.S. Pat. No. 5,810,015, “In spite of all these efforts, a need remains for efficient generation of energy to supply electrically powered implanted devices.”

The solution provided by U.S. Pat. No. 5,80,015 is described in claim 1 thereof, which describes: “An implantable power supply apparatus for supplying electrical energy to an electrically powered device, comprising: a power supply unit including: a transcutaneously, invasively rechargeable non-electrical energy storage device (NESD); an electrical energy storage device (EESD); and an energy converter coupling said NESD and said EESD, said converter including means for converting non-electrical energy stored in said NESD to electrical energy and for transferring said electrical energy to said EESD, thereby storing said electrical energy in said EESD.”

An implantable ultrasound communicaton system is disclosed in U.S. Pat. No. 5,861,018, the entire disclosure of which is hereby incorporated by reference into this specification. As is disclosed in the abstract of this patent, there is disclosed in such patent “A system for communicating through the skin of a patient, the system including an internal communication device implanted inside the body of a patient and an external communication device. The external communication device includes an external transmitter which transmits a carrier signal into the body of the patient during communication from the internal communication device to the external communication device. The internal communication device includes an internal modulator which modulates the carrier signal with information by selectively reflecting the carrier signal or not reflecting the carrier signal. The external communication device demodulates the carrier signal by detecting when the carrier signal is reflected and when the carrier signal is not reflected through the skin of the patient. When the reflected carrier signal is detected, it is interpreted as data of a first state, and when the reelected carrier signal is not detected, it is interpreted as data of a second state. Accordingly, the internal communication device consumes relatively little power because the carrier signal used to carry the information is derived from the external communication device. Further, transfer of data is also very efficient because the period needed to modulate information of either the first state or the second state onto the carrier signal is the same. In one embodiment, the carrier signal operates in the ultrasound frequency range.”

U.S. Pat. No. 5,861,019, the entire disclosure of which is hereby incorporated by reference into this specification, discloses a telemetry system for communications between an external programmer and an implantable medical device. Claim 1 of this patent describes: “A telemetry system for communications between an external programmer and an implantable medical device, comprising:the external programmer comprising an external telemetry antenna and an external transceiver for receiving uplink telemetry transmissions and transmitting downlink telemetry transmission through the external telemetry antenna; the implantable medical device comprising an implantable medical device housing, an implantable telemetry antenna and an implantable transceiver for receiving downlink transmissions and for transmitting uplink telemetry transmission through the implantable telemetry antenna, the implantable medical device housing being formed of a conductive metal and having an exterior housing surface and an interior housing surface; the implantable medical device housing being formed with a housing recess extending inwardly from the exterior housing surface to a predetermined housing recess depth in the predetermined substrate area of the exterior housing surface for receiving the dielectric substrate therein; wherein the implantable telemetry antenna is a conformal microstrip antenna formed as part of the implantable medical device housing, the microstrip antenna having electrically conductive ground plane and radiator patch layers separated by a dielectric substrate, layer the conductive radiator patch layer having a predetermined thickness and predetermined radiator patch layer dimensions, the patch layer being formed upon one side of the dielectric substrate layer.”

As is also disclosed in U.S. Pat. No. 5,861,019, “An extensive description of the historical development of uplink and downlink telemetry transmission formats” is set forth at columns 2 through 5 of U.S. Pat. No. 5,861,019. As is disclosed in these columns: “An extensive description of the historical development of uplink and downlink telemetry transmission formats and is set forth in the above-referenced '851 and '963 applications and in the following series of commonly assigned patents all of which are incorporated herein by reference in their entireties. Commonly assigned U.S. Pat. No. 5,127,404 to Grevious et al. sets forth an improved method of frame based, pulse position modulated (PPM) of data particularly for uplink telemetry. The frame-based PPM telemetry format increases bandwidth well above simple PIM or pulse width modulation (PWM) binary bit stream transmissions and thereby conserves energy of the implanted medical device. Commonly assigned U.S. Pat. No. 5,168,871 to Grevious et al. sets forth an improvement in the telemetry system of the '404 patent for detecting uplink telemetry RF pulse bursts that are corrupted in a noisy environment. Commonly assigned U.S. Pat. No. 5,292,343 to Blanchette et al. sets forth a further improvement in the telemetry system of the '404 patent employing a hand shake protocol for maintaining the communications link between the external programmer and the implanted medical device despite instability in holding the programmer RF head steady during the transmission. Commonly assigned U.S. Pat. No. 5,324,315 to Grevious sets forth an improvement in the uplink telemetry system of the '404 patent for providing feedback to the programmer to aid in optimally positioning the programmer RF head over the implanted medical device. Commonly assigned U.S. Pat. No. 5,117,825 to Grevious sets forth an further improvement in the programmer RF head for regulating the output level of the magnetic H field of the RF head telemetry antenna using a signal induced in a sense coil in a feedback loop to control gain of an amplifier driving the RF head telemetry antenna. Commonly assigned U.S. Pat. No. 5,562,714 to Grevious sets forth a further solution to the regulation of the output level of the magnetic H field generated by the RF head telemetry antenna using the sense coil current to directly load the H field. Commonly assigned U.S. Pat. No. 5,354,319 to Wybomey et al. sets forth a number of further improvements in the frame based telemetry system of the '404 patent. Many of these improvements are incorporated into MEDTRONIC® Model 9760, 9766 and 9790 programmers. These improvements and the improvements described in the above-referenced pending patent applications are directed in general to increasing the data transmission rate, decreasing current consumption of the battery power source of the implantable medical device, and increasing reliability of uplink and downlink telemetry transmissions.”

As is also disclosed in U.S. Pat. No. 5,861,019, “The current MEDTRONIC® telemetry system employing the 175 kHz carrier frequency limits the upper data transfer rate, depending on bandwidth and the prevailing signal-to-noise ratio. Using a ferrite core, wire coil, RF telemetry antenna results in: (1) a very low radiation efficiency because of feed impedance mismatch and ohmic losses; 2) a radiation intensity attenuated proportionally to at least the fourth power of distance (in contrast to other radiation systems which have radiation intensity attenuated proportionally to square of distance); and 3) good noise immunity because of the required close distance between and coupling of the receiver and transmitter RF telemetry antenna fields.”

As is also disclosed in U.S. Pat. No. 5,861,019, “These characteristics require that the implantable medical device be implanted just under the patient's skin and preferably oriented with the RF telemetry antenna closest to the patient's skin. To ensure that the data transfer is reliable, it is necessary for the patient to remain still and for the medical professional to steadily hold the RF programmer head against the patient's skin over the implanted medical device for the duration of the transmission. If the telemetry transmission takes a relatively long number of seconds, there is a chance that the programmer head will not be held steady. If the uplink telemetry transmission link is interrupted by a gross movement, it is necessary to restart and repeat the uplink telemetry transmission. Many of the above-incorporated, commonly assigned, patents address these problems.”

As is also disclosed in U.S. Pat. No. 5,861,019, “The ferrite core, wire coil, RF telemetry antenna is not bio-compatible, and therefore it must be placed inside the medical device hermetically sealed housing. The typically conductive medical device housing adversely attenuates the radiated RF field and limits the data transfer distance between the programmer head and the implanted medical device RF telemetry antennas to a few inches.”

As is also disclosed in U.S. Pat. No. 5,861,019, “In U.S. Pat. No. 4,785,827 to Fischer, U.S. Pat. No. 4,991,582 to Byers et al., and commonly assigned U.S. Pat. No. 5,470,345 to Hassler et al. (all incorporated herein by reference in their entireties), the metal can typically used as the hermetically sealed housing of the implantable medical device is replaced by a hermetically sealed ceramic container. The wire coil antenna is still placed inside the container, but the magnetic H field is less attenuated. It is still necessary to maintain the implanted medical device and the external programming head in relatively close proximity to ensure that the H field coupling is maintained between the respective RF telemetry antennas.”

As is also disclosed in U.S. Pat. No. 5,861,019, “Attempts have been made to replace the ferrite core, wire coil, RF telemetry antenna in the implantable medical device with an antenna that can be located outside the hermetically sealed enclosure. For example, a relatively large air core RF telemetry antenna has been embedded into the thermoplastic header material of the MEDTRONIC® Prometheus programmable IPG. It is also suggested that the RF telemetry antenna may be located in the IPG header in U.S. Pat. No. 5,342,408. The header area and volume is relatively limited, and body fluid may infiltrate the header material and the RF telemetry antenna.”

As is also disclosed in U.S. Pat. No. 5,861,019, “In U.S. Pat. Nos. 5,058,581 and 5,562,713 to Silvian, incorporated herein by reference in their entireties, it is proposed that the elongated wire conductor of one or more medical lead extending away from the implanted medical device be employed as an RF telemetry antenna. In the particular examples, the medical lead is a cardiac lead particularly used to deliver energy to the heart generated by a pulse generator circuit and to conduct electrical heart signals to a sense amplifier. A modest increase in the data transmission rate to about 8 Kb/s is alleged in the '581 and '713 patents using an RF frequency of 10-300 MHz. In these cases, the conductor wire of the medical lead can operate as a far field radiator to a more remotely located programmer RF telemetry antenna. Consequently, it is not necessary to maintain a close spacing between the programmer RF telemetry antenna and the implanted cardiac lead antenna or for the patient to stay as still as possible during the telemetry transmission.”

As is also disclosed in U.S. Pat. No. 5,861,019, “However, using the medical lead conductor as the RF telemetry antenna has several disadvantages. The radiating field is maintained by current flowing in the lead conductor, and the use of the medical lead conductor during the RF telemetry transmission may conflict with sensing and stimulation operations. RF radiation losses are high because the human body medium is lossy at higher RF frequencies. The elongated lead wire RF telemetry antenna has directional radiation nulls that depend on the direction that the medical lead extends, which varies from patient to patient. These considerations both contribute to the requirement that uplink telemetry transmission energy be set artificially high to ensure that the radiated RF energy during the RF uplink telemetry can be detected at the programmer RF telemetry antenna. Moreover, not all implantable medical devices have lead conductor wires extending from the device.”

As is also disclosed in U.S. Pat. No. 5,861,019, “A further U.S. Pat. No. 4,681,111 to Silvian, incorporated herein by reference in its entirety, suggests the use of a stub antenna associated with the header as the implantable medical device RF telemetry antenna for high carrier frequencies of up to 200 MHz and employing phase shift keying (PSK) modulation. The elimination of the need for a VCO and a bit rate on the order of 2-5% of the carrier frequency or 3.3-10 times the conventional bit rate are alleged.”

As is also disclosed in U.S. Pat. No. 5,861,019, “At present, a wide variety of implanted medical devices are commercially released or proposed for clinical implantation. Such medical devices include implantable cardiac pacemakers as well as implantable cardioverter-defibrillators, pacemaker-cardioverter-defibrillators, drug delivery pumps, cardiomyostimulators, cardiac and other physiologic monitors, nerve and muscle stimulators, deep brain stimulators, cochlear implants, artificial hearts, etc. As the technology advances, implantable medical devices become ever more complex in possible programmable operating modes, menus of available operating parameters, and capabilities of monitoring increasing varieties of physiologic conditions and electrical signals which place ever increasing demands on the programming system.”

As is also disclosed in U.S. Pat. No. 5,861,019, “It remains desirable to minimize the time spent in uplink telemetry and downlink transmissions both to reduce the likelihood that the telemetry link may be broken and to reduce current consumption.”

As is also disclosed in U.S. Pat. No. 5,861,019, “Moreover, it is desirable to eliminate the need to hold the programmer RF telemetry antenna still and in proximity with the implantable medical device RF telemetry antenna for the duration of the telemetry transmission. As will become apparent from the following, the present invention satisfies these needs.”

The solution to this problem is presented, e.g., in claim 1 of U.S. Pat. No. 5,861,019. This claim describes “A telemetry system for communications between an external programmer and an implantable medical device, comprising: the external programmer comprising an external telemetry antenna and an external transceiver for receiving uplink telemetry transmissions and transmitting downlink telemetry transmission through the external telemetry antenna; the implantable medical device comprising an implantable medical device housing, an implantable telemetry antenna and an implantable transceiver for receiving downlink transmissions and for transmitting uplink telemetry transmission through the implantable telemetry antenna, the implantable medical device housing being formed of a conductive metal and having an exterior housing surface and an interior housing surface; the implantable medical device housing being formed with a housing recess extending inwardly from the exterior housing surface to a predetermined housing recess depth in the predetermined substrate area of the exterior housing surface for receiving the dielectric substrate therein; wherein the implantable telemetry antenna is a conformal microstrip antenna formed as part of the implantable medical device housing, the microstrip antenna having electrically conductive ground plane and radiator patch layers separated by a dielectric substrate, layer the conductive radiator patch layer having a predetermined thickness and predetermined radiator patch layer dimensions, the patch layer being formed upon one side of the dielectric substrate layer.”

U.S. Pat. No. 5,945,762, the entire disclosure of which is hereby incorporated by reference into this specification, discloses an external transmitter adapted to magnetically excite an implanted receiver coil. Claim 1 of this patent describes “An external transmitter adapted for magnetically exciting an implanted receiver coil, causing an electrical current to flow in the implanted receiver coil, comprising: (a) a support; (b) a magnetic field generator that is mounted to the support; and (c) a prime mover that is drivingly coupled to an element of the magnetic field generator to cause said element of the magnetic field generator to reciprocate, in a reciprocal motion, said reciprocal motion of said element of the magnetic field generator producing a varying magnetic field that is adapted to induce an electrical current to flow in the implanted receiver coil.”

U.S. Pat. No. 5,954,758, the entire disclosure of which is hereby incorporated by reference into this specification, claims an implantable electrical stimulator comprised of an implantable radio frequency receiving coil, an implantable power supply, an implantable input singal generator, an implantable decoder, and an implantable electrical stimulator. Claim 1 of this patent describes “A system for transcutaneously telemetering position signals out of a human body and for controlling a functional electrical stimulator implanted in said human body, said system comprising: an implantable radio frequency receiving coil for receiving a transcutaneous radio frequency signal; an implantable power supply connected to said radio frequency receiving coil, said power supply converting received transcutaneous radio frequency signals into electromotive power; an implantable input signal generator electrically powered by said implantable power supply for generating at least one analog input movement signal to indicate voluntary bodily movement along an axis; an implantable encoder having an input operatively connected with said implantable input signal generator for encoding said movement signal into output data in a preselected data format; an impedance altering means connected with said encoder and said implantable radio frequency signal receiving coil to selectively change an impedance of said implantable radio frequency signal receiving coil; an external radio frequency signal transmit coil inductively coupled with said implantable radio frequency signal receiving coil, such that impedance changes in said implantable radio frequency signal receiving coil are sensed by said external radio frequency signal transmit coil to establish a sensed modulated movement signal in said external transmit coil; an external control system electrically connected to said external radio frequency transmit coil for monitoring said sensed modulated movement signal in said external radio frequency transmit coil, said external control system including: a demodulator for recovering the output data of said encoder from the sensed modulated ovement signal of said external transmit coil,a pulse width algorithm means for applying a preselected pulse width algorithm to the recovered output data to derive a first pulse width,an amplitude algorithm means for applying an amplitude algorithm to the recovered output data to derive a first amplitude therefrom,an interpulse interval algorithm means for applying an interpulse algorithm to the recovered output data to derive a first interpulse interval therefrom; and,a stimulation pulse train signal generator for generating a stimulus pulse train signal which has the first pulse width and the first pulse amplitude;an implantable functional electrical stimulator for receiving said stimulation pulse train signal from said stimulation pulse train signal generator and generating stimulation pulses with the first pulse width, the first pulse amplitude, and separated by the first interpulse interval; and, at least one electrode operatively connected with the functional electrical stimulator for applying said stimulation pulses to muscle tissue of said human body.”

U.S. Pat. No. 6,006,133, the entire disclosure of which is hereby incorporated by reference into this specification, describes an implantable medical device comprised of a hermetically sealed housing.

U.S. Pat. No. 6,083,166, the entire disclosure of which is hereby incorporated by reference into this specification, discloses an ultrasound transmitter for use with a surgical device.

U.S. Pat. No. 6,152,882, the entire disclosure of which is hereby incorporated by reference into this specification, discloses an implantable electroporation unit, an implantable proble electrode, an implantable reference electrode, and an an amplifier unit. Claim 35 of this patent describes: “Apparatus for measurement of monophasic action potentials from an excitable tissue including a plurality of cells, the apparatus comprising: at least one probe electrode placeable adjacent to or in contact with a portion of said excitable tissue; at least one reference electrode placeable proximate said at least one probe electrode; an electroporating unit electrically connected to said at least one probe electrode and said at least one reference electrode for controllably applying to at least some of said cells subjacent said at least one probe electrode electrical current pulses suitable for causing electroporation of cell membranes of said at least some of said cells; and an amplifier unit electrically connected to said at least one probe electrode and to said at least one reference electrode for providing an output signal representing the potential difference between said probe electrode and said reference electrode”

U.S. Pat. No. 6,169,925, the entire disclosure of which is hereby incorporated by reference into this specification, describes a transceiver for use in communication with an implantable medical device. Claim 1 of this patent describes: “An external device for use in communication with an implantable medical device, comprising: a device controller; a housing; an antenna array mounted to the housing; an RF transceiver operating at defined frequency, coupled to the antenna array; means for encoding signals to be transmitted to the implantable device, coupled to an input of the transceiver; means for decoding signals received from the implantable device, coupled to an output of the transceiver; and means for displaying the decoded signals received from the implantable device; wherein the antenna array comprises two antennas spaced a fraction of the wavelength of the defined frequency from one another, each antenna comprising two antenna elements mounted to the housing and located orthogonal to one another; and wherein the device controller includes means for selecting which of the two antennas is coupled to the transceiver.”

U.S. Pat. No. 6,185,452, the entire disclosure of which is hereby incorporated by reference into this specification, claims a device for stimulating internal tissue, wherein such device is comprised of: “a sealed elongate housing configured for implantation in said patient's body, said housing having an axial dimension of less than 60 mm and a lateral dimension of less than 6 mm; power consuming circuitry carried by said housing including at least one electrode extending externally of said housing, said power consuming circuitry including a capacitor and pulse control circuitry for controlling (1) the charging of said capacitor and (2) the discharging of said capacitor to produce a current pulse through said electrode; a battery disposed in said housing electrically connected to said power consuming circuitry for powering said pulse control circuitry and charging said capacitor, said battery having a capacity of at least one microwatt-hour; an internal coil and a charging circuit disposed in said housing for supplying a charging current to said battery; an external coil adapted to be mounted outside of said patient's body; and means for energizing said external coil to generate an alternating magnetic field for supplying energy to said charging circuit via said internal coil.”

U.S. Pat. No. 6,235,024, the entire disclosure of which is hereby incorporated by reference into this specification, discloses an implantable high frequency energy generator. Claim 1 of this patent describes: “A catheter system comprising: an elongate catheter tubing having a distal section, a distal end, a proximal end, and at least one lumen extending between the distal end and the proximal end; a handle attached to the proximal end of said elongate catheter tubing, wherein the handle has a cavity; an ablation element mounted at the distal section of the elongate catheter tubing, the ablation element having a wall with an outer surface and an inner surface, wherein the outer surface is covered with an outer member made of a first electrically conductive material and the inner surface is covered with an inner member made of a second electrically conductive material, and wherein the wall comprises an ultrasound transducer; an electrical conducting means having a first and a second electrical wires, wherein the first electrical wire is coupled to the outer member and the second electrical wire is coupled to the inner member of the ablation element; and a high frequency energy generator means for providing a radiofrequency energy to the ablation element through a first electrical wire of the electrical conducting means.”

An implantable light-generating apparatus is described in claim 16 of U.S. Pat. No. 6,363,279, the entire disclosure of which is hereby incorporated by reference into this specification. As is disclosed in such claim 16, this patent provides a “Heart control apparatus, comprising circuitry for generating a non-excitatory stimulus, and stimulus application devices for applying to a heart or to a portion thereof said non-excitatory stimulus, wherein the circuitry for generating a non-excitatory stimulus generates a stimulus which is unable to generate a propagating action potential and wherein said circuitry comprises a light-generating apparatus for generating light.

An implantable ultrasound probe is described in claim 1 of U.S. Pat. No. 6,421,565, the entire disclosure of which is hereby incorporated by reference into this specifcation. This claim 1 describes “An implantable cardiac monitoring device comprising: an A-mode ultrasound probe adapted for implantation in a right ventricle of a heart, said ultrasound probe emitting an ultrasound signal and receiving at least one echo of said ultrasound signal from at least one cardiac segment of the left ventricle; a unit connected to said ultrasound probe for identifying a time difference between emission of said ultrasound signal and reception of said echo and, from said time difference, determining a position of said cardiac segment, said cardiac segment having a position which, at least when reflecting said ultrasound signal, is correlated to cardiac performance, and said unit deriving an indication of said cardiac performance from said position of said cardiac segment.”

An implantalbe stent that contains a tube and several optical emitters located on the innser surface of the tube is disclosed in U.S. Pat. No. 6,488,704, the entire disclosure of which is hereby incorporated by reference into this specification. Claim 1 of this patent describes “1. An implantable stent which comprises: (a) a tube comprising an inner surface and an outer surface, and (b) a multiplicity of optical radiation emitting means adapted to emit radiation with a wavelength from about 30 nanometers to about 30 millimeters, and a multiplicity of optical radiation detecting means adapted to detect radiation with a wavelength of from about 30 nanometers to about 30 millimeters, wherein said optical radiation emitting means and said optical radiation detecting means are disposed on the inside surface of said tube.”

Many other implantable devices and configurations are described in the claims of U.S. Pat. No. 6,488,704.

Thus, e.g., claim 2 of such patent disloses that the “ . . . implantable stent is comprised of a flexible casing with an inner surface and an outer surface.” Claim 3 of such patent discloses that the case may be “ . . . comprised of fluoropolymer.” Claim 4 of such patent discloses that the casing may be “ . . . optically impermeable.”

Thus, e.g., claim 10 of U.S. Pat. No. 6,488,704 discloses an embodiment in which an implantable stent contains “ . . . telemetry means for transmitting a signal to a receiver located external to said implantable stent.” The telemetry means may be adated to receive “ . . . a signal from a transmitter located external to said implantable stent (see claim 11); and such signal may be a radio-frequency signal (see claims 12 and 13). The implantable stent may also comprise “ . . . telemetry means for transmitting a signal to a receiver located external to said implantable stent” (see claim 22), and/or “ . . . telemetry means for receiving a signal from a transmitter located external to said implantable stent” (see claim 23), and/or “ . . . a controller operatively connected to said means for transmitting a signal to said receiver, and operatively connected to said means for receiving a signal from said transmitter” (see claim 24).

Thus, e.g., claim 14 of U.S. Pat. No. 6,488,704 describes an implantable stent that contains a waveguide array. The waveguide array may contain “ . . . a flexible optical waveguide device” (see claim 15), and/or “ . . . means for transmitting optical energy in a specified configuration” (see claim 16), and/or “ . . . a waveguide interface for receiving said optical energy transmitted in said specified configuration by said waveguide array” (see claim 17), and/or “ . . . means for filtering specified optical frequencies” (see claim 18). The implantalbe stent may be comprised of “ . . . means for receiving optical energy from said waveguide array” (see claim 19), and/or “ . . . means for processing said optical energy received from waveguide array” (see claim 20). The implantable stent may comprise “ . . . means for processing said radiation emitted by said optical radiation emitting means adapted with a wavelength from about 30 nanometers to about 30 millimeters” (see claim 21).

The implantable stent may be comprised of implantable laser devices. Thus, e.g., and referring again to U.S. Pat. No. 6,488,704, the implantable stent may be comprised of “ . . . a multiplicity of vertical cavity surface emitting lasers and photodetectors arranged in a monolithic configuration” (see claim 27), wherein “ . . . said monolithic configuration further comprises a multiplicity of optical drivers operatively connected to said vertical cavity surface emitting lasers” (see claim 28) and/or wherein “ . . . said vertical cavity surface emitting lasers each comprise a multiplicity of distributed Bragg reflector layers” (see claim 29), and/or wherein “ . . . each of said photodetectors comprises a multiplicity of distributed Bragg reflector layers” (see claim 30), and/or wherein “ . . . each of said vertical cavity surface emitting lasers is comprised of an emission layer disposed between a first distributed Bragg reflector layer and a second distributed Bragg reflector layer” (see claim 31), and/or wherein “ . . . said emission layer is comprised of a multiplicity of quantum well structures” (see claim 32), and/or wherein “ . . . each of said photodetectors is comprised of an absorption layer disposed between a first distributed Bragg reflector layer and a second distributed Bragg reflector layer” (see claim 33), and/or wherein “ . . . each of said vertical cavity surface emitting lasers and photodetectors is disposed on a separate semiconductor substrate” (see claim 34), and/or wherein “ . . . said semiconductor substrate comprises gallium arsenide.”

Referring again to U.S. Pat. No. 6,488,704, the entire disclosure of which is hereby incorporated by reference into this specification, the implantable stent may be comprised of an arithmetic unit (see claim 37 of such patent), and such arithmetic unit may be “ . . . comprised of means for receiving signals from said optical radiation detecting means” (see claim 38), and/or “ . . . means for calculating the concentration of components in an analyte disposed within said implantable stent (see claim 39). In one embodiment, “said means for calculating the concentration of components in said analyte calculates concentrations of said components in said analyte based upon optimum optical path lengths for different wavelengths and values of transmitted light (see claim 40).

Referring again to U.S. Pat. No. 6,488,704, the implantalbe stent may contain a power supply (see claim 41 thereof) which may contain a battery (see claim 42) which, in one embodiment, is a lithium-iodine battery (see claim 43).

U.S. Pat. No. 6,585,763, the entire disclosure of which is hereby incorporated by reference into this specification, describes in its claim 1 “ . . . a vascular graft comprising: a biocompatible material formed into a shape having a longitudinal axis to enclose a lumen disposed along said longitudinal axis of said shape, said lumen positioned to convey fluid through said vascular graft; a first transducer coupled to a wall of said vascular graft; and an implantable circuit for receiving electromagnetic signals, said implantable circuit coupled to said first transducer, said first transducer configured to receive a first energy from said circuit to emit a second energy having one or more frequencies and power levels to alter said biological activity of said medication in said localized area of said body subsequent to implantation of said first transducer in said body near said localized area.” The transducer may be selected from the group consisting of “ . . . an ultrasonic transducer, a plurality of light sources, an electric field transducer, an electromagnetic transducer, and a resistive heating transducer” (see claim 2), it may comprise a coil (see claim 3), it may comprise “ . . . a regular solid including piezoelectric material, and wherein a first resonance frequency, being of said one or more frequencies, is determined by a first dimension of said regular solid and a second resonance frequency, being of said one or more frequencies, is determined by a second dimension of said regular solid and further including a first electrode coupled to said regular solid and a second electrode coupled to said regular solid” (see claim 4).

U.S. Pat. No. 6,605,089, the entire disclosure of which is hereby incorporated by reference into this specification, discloses an implantable bone growth promoting device. Claim 1 of this patent describes “A device for placement into and between at least two adjacent bone masses to promote bone growth therebetween, said device comprising: an implant having opposed first and second surfaces for placement between and in contact with the adjacent bone masses, a mid-longitudinal axis, and a hollow chamber between said first and second surfaces, said hollow chamber being adapted to hold bone growth promoting material, said hollow chamber being along at least a portion of the mid-longitudinal axis of said implant, each of said first and second surfaces having at least one opening in communication with said hollow chamber into which bone from the adjacent bone masses grows; and an energizer for energizing said implant, said energizer being sized and configured to promote bone growth from adjacent bone mass to adjacent bone mass through said first and second surfaces and through at least a portion of said hollow chamber at the mid-longitudinal axis.” The implant may have a coil wrapped around it (see claim 6), a portion of the coil may be “ . . . in the form of an external thread on at least a portion of said first and second surfaces of said implant” (see claim 7), the “external thread” may be energized by the “energizer” (claim 8) by conducting “ . . . electromagnetic energy to said interior space . . . ” of the energizer (claim 9).

Referring again to U.S. Pat. No. 6,605,089, and to the implant claimed therein, the implant may contain “ . . . a power supply delivering an electric charge” (see claim 14), and it may comprise “ . . . a first portion that is electrically conductive for delivering said electrical charge to at least a portion of the adjacent bone masses and said energizer delivers negative electrical charge to said first portion of said implant” (see claim 15). Additionally, the implant may also contain “ . . . a controller for controlling the delivery of said electric charge” that is disposed within the implant (see claim 18), that “ . . . includes one of a wave form generator and a voltage generator” (see claim 19), and that “ . . . provides for the delivery of one of an alternating current, a direct current, and a sinusoidal current” (see claim 21).

U.S. Pat. No. 6,641,520, the entire disclosure of which is hereby incorporated by reference into this specification,discloses a magnetic field generator for providing a static or direct durrent magnetic field generator. In column 1 of this patent, some “prior art” magnetic field generators were described. It was stated in such column 1 that: “There has recently been an increased interest in therapeutic application of magnetic fields. There have also been earlier efforts of others in this area. The recent efforts, as well as those earlier made, can be categorized into three general types, based on the mechanism for generating and applying the magnetic field. The first type were what could be generally referred to as systemic applications. These were large, tubular mechanisms which could accommodate a human body within them. A patient or recipient could thus be subjected to magnetic therapy through their entire body. These systems were large, cumbersome and relatively immobile. Examples of this type of therapeutic systems included U.S. Pat. Nos. 1,418,903; 4,095,588; 5,084,003; 5,160,591; and 5,437,600. A second type of system was that of magnetic therapeutic applicator systems in the form of flexible panels, belts or collars, containing either electromagnets or permanent magnets. These applicator systems could be placed on or about portion of the recipient's body to allow application of the magnetic therapy. Because of their close proximity to the recipients body, considerations limited the amount and time duration of application of magnetic therapy. Examples of this type system were U.S. Pat. Nos. 4,757,804; 5,084,003 and 5,344,384. The third type of system was that of a cylindrical or toroidal magnetic field generator, often small and portable, into which a treatment recipient could place a limb to receive electromagnetic therapy. Because of size and other limitations, the magnetic field strength generated in this type system was usually relatively low. Also, the magnetic field was a time varying one. Electrical current applied to cause the magnetic field was time varying, whether in the form of simple alternating current waveforms or a waveform composed of a series of time-spaced pulses.”

The magnetic field generator claimed in U.S. Pat. No. 6,641,520 comprised “ . . . a magnetic field generating coil composed of a wound wire coil generating the static magnetic field in response to electrical power; a mounting member having the coil mounted thereon and having an opening therethrough of a size to permit insertion of a limb of the recipient in order to receive electromagnetic therapy from the magnetic field coil; an electrical power supply furnishing power to the magnetic field coil to cause the coil to generate a static electromagnetic field within the opening of the mounting member for application to the recipient's limb; a level control mechanism providing a reference signal representing a specified electromagnetic field strength set point for regulating the power furnished to the magnetic field coil; a field strength sensor detecting the static electromagnetic field strength generated by the magnetic field coil and forming a field strength signal representing the detected electromagnetic field strength in the opening in the mounting member; a control signal generator receiving the field strength signal from the field strength sensor and the reference signal from the level control mechanism representing a specified electromagnetic field strength set point; and the control signal generator forming a signal to regulate the power flowing from the electrical power supply to the magnetic field coil.”

An implantable sensor is disclosed in U.S. Pat. No. 6,491,639, the entire disclosure of which is hereby incorporated by reference into this specification. Claim 1 of such patent describes: “An implantable medical device including a sensor for use in detecting the hemodynamic status of a patient comprising:a hermetic device housing enclosing device electronics for receiving and processing data; and said device housing including at least one recess and a sensor positioned in said at least one recess.” Claim 10 of such patent describes “10. An implantable medical device including a hemodynamic sensor for monitoring arterial pulse amplitude comprising: a device housing; a transducer comprising a light source and a light detector positioned exterior to said device housing responsive to variations in arterial pulse amplitude; and wherein said light detector receives light originating from said light source and reflected from arterial vasculature of a patient and generates a signal which is indicative of variations in the reflected light caused by the expansion and contraction of said arterial vasculature. “Claim 14 of such patent describes: “14. An implantable medical device including a hemodynamic sensor for monitoring arterial pulse amplitude comprising: a device housing; and an ultrasound transducer associated with said device housing responsive to variations in arterial pulse amplitude.” Claim 15 of such patent describes: “15. An implantable medical device including a hemodynamic sensor for monitoring arterial pulse amplitude comprising: a device housing; and a transducer associated with said device housing responsive to variations in arterial pulse amplitude, said device housing having at least one substantially planar face and said transducer is positioned on said planar face.” Claim 17 of such patent describes “ . . . an implantable pulse generator . . . ’

U.S. Pat. No. 6,663,555, the entire disclosure of which is incorporated by reference into this specification, also claims a magnetic field generator. Claim 1 of this patent describes: “A magnet keeper-shield assembly for housing a magnet, said magnet keeper-shield assembly comprising: a keeper-shield comprising a material substantially permeable to a magnetic flux; a cavity in the keeper-shield, said cavity comprising an inner side wall and a base, and said cavity being adapted to accept a magnet having a front and a bottom face; an actuator extending through the base; a plurality of springs extending through the base, said springs operative to exert a force in a range from about 175 pounds to about 225 pounds on the bottom face of the magnet in a retracted position, and wherein said magnet produces at least about 118 gauss at a distance of about 10 cm from the front face in the extended position and produces at most about 5 gauss at a distance less than or equal to about 22 cm from the front face in the retracted position.”

Published U.S. patent application US2002/0182738 discloses an implantable flow cytometer the entire disclosure of this published United States patent application is hereby incorporated by reference into this specification. Claim 1 of this patent describes “A flow cytometer comprising means for sampling cellular material within a body, means for marking cells within said bodily fluid with a marker to produce marked cells, means for analyzing said marked cells, a first means for removing said marker from said marked cells, a second means for removing said marker from said marked cells, means for sorting said cells within said bodily fluid to produce sorted cells, and means for maintaining said sorted cells cells in a viable state.”

Referring again to published U.S. patent application US 2002/0182738, the implantable flow cytometer may contain “ . . . a a first control valve operatively connected to said first means for removing said marker from said marked cells and to said second means for removing said marker from said marked cells . . . ” (see claim 3), a controller connected to the first control valve (claim 4), a second control valve (claim 5), a third control valve (claim 6), a dye separator (claims 7 and 8), an analyzer for testing blood purity (claim 9), etc.

A similar flow cytometer is disclosed in published U.S. patent application US 2003/0036718, the entire disclosure of which is also hereby incorporated by reference into this specification.

Published U.S. patent application US 2003/0036776, the entire disclosure of which is hereby incorporated by reference into this specification, discloses an MRI-compatible implantable device. Claim 1 of this patent describes “A cardiac assist device comprising means for connecting said cardiac assist device to a heart, means for furnishing electrical impulses from said cardiac assist device to said heart, means for ceasing the furnishing of said electrical impulses to said heart, means for receiving pulsed radio frequency fields, means for transmitting and receiving optical signals, and means for protecting said heart and said cardiac assist device from currents induced by said pulsed radio frequency fields, wherein said cardiac assist device contains a control circuit comprised of a parallel resonant frequency circuit and means for activating said parallel resonant frequency circuit.” The “ . . . means for activating said parallel resonant circuit . . . .” may contain “ . . . comprise optical means (see claim 2) such as an optical switch (claim 3) comprised of “ . . . a pin type diode . . . ” (claim 4) and connected to an optical fiber (claim 5). The optical switch may be “ . . . activated by light from a light source . . . ” (claim 6), and it may be located with a biological organism (claim 7). The light source may be located within the biological organism (claim 9), and it may provide “ . . . light with a wavelength of from about 750 to about 850 nanometers . . . .”

Other Compositions Comprised of Nanomagnetic Particles

In addition to the compositions already mentioned in this specification, other compositions may advantageous incorporate the nanomagnetic material of this invention. Thus, by way of illustration and not limitation, one may replace the magnetic particles in prior art compositions with the nanomagnetic materials of this invention.

In many of the prior art patents, the term “comprising magnetic particles” appears in the claims; some of these patents are described below. In the compositions and processes described in the patents described below, one may replace the “magnetic particles” used in such patents with the nanomagnetic particles of this invention. Thus, e.g., one may use such nanomagnetic particles in the compositions and processes of U.S. Pat. No. 3,777,295 (magnetic particle core), U.S. Pat. No. 3,905,841 (magnetic particles disposed in organic resin binders), U.S. Pat. No. 4,0188,886 (protein-coated magnetic particles), U.S. Pat. No. 4,145,300 (developers containing magnetic particles and a sublimable dyestuff), U.S. Pat. No. 4,171,274 (tessellated magnetic particles), U.S. Pat. No. 4,177,089 (magnetic particles and compacts thereof), U.S. Pat. No. 4,177,253 (magnetic particles for immunoassay), U.S. Pat. No. 4,189,514 (high-temperature magnetic tape), U.S. Pat. No. 4,197,563 (magnetic particles disposed in a polymerizable ink), U.S. Pat. No. 4,271,782 (apparatus for disorienting magnetic particles), U.S. Pat. No. 4,283,476 (photographic element having a magnetic recording stripe), U.S. Pat. No. 4,379,183 (cobalt-modified magnetic particles), U.S. Pat. No. 4,382,982 (process for protecting magnetic particles with chromium oxide), U.S. Pat. No. 4,419,383 (method for individually encapsulating magnetic particles), U.S. Pat. No. 4,433,289 (mixture of magnetic particles and a water soluble carrier solid), U.S. Pat. No. 4,438,179 (resin particles with magnetic particles bonded to surface), U.S. Pat. No. 4,448,870 (magnetic color toner), U.S. Pat. No. 4,486,523 (magnetic toner particles coated with opaque polymer particles), U.S. Pat. No. 4,505,990 (coating compositions), U.S. Pat. No. 4,532,153 (method of bonding magnetic particles to a resin particles), U.S. Pat. No. 4,546,035 (polymeric additives for magnetic coating materials), U.S. Pat. No. 4,628,037 (binding assays employing magnetic particles), U.S. Pat. No. 4,638,032 (magnetic particles as supports for organic synthesis), U.S. Pat. No. 4,651,092 (resin/solvent mixture containing magnetic particles), U.S. Pat. No. 4,698,302 (enzymatic reactions using magnetic particles), U.S. Pat. No. 4,701,024 (liquid crystal material including magnetic particles), U.S. Pat. No. 4,707,523 (magnetic particles), U.S. Pat. No. 4,728,363 (acicular magnetic particles), U.S. Pat. No. 4,731,337 (fluorometric immunological assay with magnetic particles), U.S. Pat. No. 4,777,145 (immunological assay method using magnetic particles), U.S. Pat. No. 4,857,417 (cobalt-containing magnetic particles), U.S. Pat. No. 4,882,224 (magnetic particles, method for making, and an electromagnetic clutch using the same), U.S. Pat. No. 5,001,424 (measurement of magnetic particles suspended in a fluid), U.S. Pat. No. 5,019,272 (filters having magnetic particles thereon), U.S. Pat. No. 5,021,315 (magnetic particles with improved conductivity), U.S. Pat. No. 5,051,200 (flexible high energy magnetic blend compositions based on rare earth magnetic particles in highly saturated nitrile rubber), U.S. Pat. No. 5,061,571 (magnetic recording medium comprising magnetic particles and a polyester resin), U.S. Pat. No. 5,071,724 (method for making colored magnetic particles), U.S. Pat. No. 5,082,733 (magnetic particles surface treated with a glycidyl compound), U.S. Pat. No. 5,104,582 (electrically conductive fluids), U.S. Pat. No. 5,142,001 (polyurethane composition), U.S. Pat. No. 5,158,871 (method of using magnetic particles for isolating, collecting, and assaying diagnostic ligates), U.S. Pat. No. 5,178,953 (magnetic recording media), U.S. Pat. No. 5,180,650 (toner compositions with conductive colored magnetic particles between core segments), U.S. Pat. No. 5,204,653 (electromagnetic induction device with magnetic particles between core segments), U.S. Pat. No. 5,209,946 (gelatin containing magnetic particles), U.S. Pat. No. 5,217,804 (magnetic particles), U.S. Pat. No. 5,230,964 (magnetic particle binder), U.S. Pat. No. 5,242,837 (light attenuating magnetic particles), U.S. Pat. No. 5,264,157 (an electronic conductive polymer incorporating magnetic particles), U.S. Pat. No. 5,316,699 (magnetic particles dispersed in a dielectric matrix), U.S. Pat. No. 5,328,793 (magnetic particles for magnetic toner), U.S. Pat. No. 5,330,669 (magnetic coating formulations), U.S. Pat. No. 5,350,676 (method for performing fibrinogen assays using dry chemical reagents containing magnetic particles), U.S. Pat. No. 5,362,027 (flow regulating valve for magnetic particles), U.S. Pat. No. 5,371,166 (polyurethane composition), U.S. Pat. No. 5,384,535 (electric magnetic detector of magnetic particles in a steam of fluid), U.S. Pat. No. 5,405,743 (reversible agglutination mediators), U.S. Pat. No. 5,428,332 (magnetized material having enhanced magnetic pull strength), U.S. Pat. No. 5,441,746 (electromagnetic wave absorbing, surface modified magnetic particles for use in medical applications), U.S. Pat. No. 5,443,654 (ferrofluid paint removal system), U.S. Pat. No. 5,445,881 (magnetic tape), U.S. Pat. No. 5,508,164 (isolation of biological materials using magnetic particles), U.S. Pat. No. 5,512,332 (process of making resuspendable coated magnetic particles), U.S. Pat. No. 5,512,439 (oligonucleotide-linked magnetic particles), U.S. Pat. No. 5,543,219 (encapsulated magnetic particles pigments), U.S. Pat. No. 5,670,077 (aqueous magnetorheological materials), U.S. Pat. No. 5,843,567 (electrical component containing magnetic particles), U.S. Pat. No. 5,843,579 (magnetic thermal transfer ribbon with aqueous ferroflids), U.S. Pat. No. 5,855,790 (magnetic particles for use in the purification of solutions), U.S. Pat. No. 5,858,595 (magnetic toner and ink jet compositions), U.S. Pat. No. 5,861,285 (fusion protein-bound magnetic particles), U.S. Pat. No. 5,898,071 (DNA purification and isolation using magnetic particles), U.S. Pat. No. 5,932,097 (microfabricated magnetic particles for applications to affinity binding), U.S. Pat. No. 5,919,490 (preparation for improving the blood supply containing hard magnetic particles), U.S. Pat. No. 5,935,886 (preparation of molecular magnetic switches), U.S. Pat. No. 5,938,979 (electromagnetic shielding), U.S. Pat. No. 5,981,095 (magnetic composites and methods for improved electrolysis), U.S. Pat. No. 5,945,525 (method for isolating nucleic acids using silica-coated magnetic particles), U.S. Pat. No. 5,958,706 (fine magnetic particles containing useful proteins bound thereto), U.S. Pat. No. 6,033,878 (protein-bound magnetic particles), U.S. Pat. No. 6,045,901 (magnetic recording medium), U.S. Pat. No. 6,090,517 (two component type developer for electrostatic latent image), U.S. Pat. No. 6,096,466 (developer), U.S. Pat. No. 6,099,999 (binder carrier comprising magnetic particles and resin), U.S. Pat. No. 6,130,019 (binder carrier), U.S. Pat. No. 6,157,801 (magnetic particles for charging), U.S. Pat. No. 6,165,795 (methods for performing fibrinogen assays using chemical reagents containing ecarin and magnetic particles), U.S. Pat. No. 6,174,661 (silver halide photographic elements), U.S. Pat. No. 6,190,573 (extrusion-molded magnetic body), U.S. Pat. No. 6,203,487 (use of magnetic particles in the focal delivery of cells), U.S. Pat. No. 6,204,033 (polyvinyl alcohol-based magnetic particles for binding biomolecules), U.S. Pat. No. 6,207,003 (fabrication of sturcutre having structural layers and layers of controllable electricalor magnetic properties), U.S. Pat. No. 6,207,313 (magnetic composites), U.S. Pat. No. 6,210,572 (filter comprised of magnetic particles), U.S. Pat. No. 6,231,760 (apparatus for mxing and separation employing magnetic particles), U.S. Pat. No. 6,274,386 (reagent preparation containing magnetic particles in tablet form), U.S. Pat. No. 6,280,618 (multiplex flow assays with magnetic particles as solid phase), U.S. Pat. No. 6,297,062 (separation by magnetic particles), U.S. Pat. No. 6,285,848 (toner), U.S. Pat. No. 6,315,709 (magnetic vascular defect treatement system), U.S. Pat. No. 6,344,273 (treatment solution for forming insulating layers on magnetic particles, process of forming the insulating layers, and electric device with a soft magnetic powder composite core), U.S. Pat. No. 6,337,215 (magnetic particles having two antiparallel ferromagnetic layers and attached affinity recognition molecules), U.S. Pat. No. 6,348,318 (methods for concentrating ligands using magnetic particles), U.S. Pat. No. 6,368,800 (kits for isolating biological target materials using silica magnetic particles), U.S. Pat. No. 6,372,338 (spherical magnetic particles for magnetic recording media), U.S. Pat. No. 6,372,517 (magnetic particles with biologically active receptors), U.S. Pat. No. 6,402,978 (magnetic polishing fluids), U.S. Pat. No. 6,405,007 (magnetic particles for charging), U.S. Pat. No. 6,464,968 (magnetic fluids), U.S. Pat. No. 6,479,302 (method for the immunological determination of an analyte), U.S. Pat. No. 6,527,972 (magnetorehologoical polymer gels), U.S. Pat. No. 6,521,341 (magnetic particles for separating molecules), U.S. Pat. No. 6,545,143 (magnetic particles for purifying nucleic acids), U.S. Pat. No. 6,569,530 (magnetic recording medium), U.S. Pat. No. 6,639,291 (spin dependent tunneling barriers doped with magnetic particles), U.S. Pat. No. 6,705,874 (colored magnetic particles), and the like. The entire disclosure of each and every one of these United States patent applications is hereby incorporated by reference into this specification.

By way of further illustration, one may substitute applicants' nanomagnetic particles for the magnetic particles used in prior art drug formulations.

By way of yet further illustration, one may replace “magnetic particles” described in the medical device claimed in published United States patent application 2004/0030379 with applicants' nanomagnetic particles. The entire disclosure of published U.S. patent application US 2004/0030379 is hereby incorporated by reference into this specification.

Published U.S. patent application US 2004/0030379 claims, in its claim 1, “A medical device that is insertable into the body of a patient comprising: (a) a surface; (b) a first coating layer comprising a biologically active material disposed on at least a portion of the surface; and (c) a second coating layer comprising a polymeric material and magnetic particles disposed on the first coating layer, wherein the second coating layer is substantially free of the biologically active material.” Claim 15 of ublished U.S. patent application US 2004/0030379 claims: “15. A system for delivering a biologically active material to a patient comprising: (a) a medical device that is insertable into the body of the patient which comprises a surface; a first coating layer comprising a biologically active material disposed on at least a portion of the surface; and a second coating layer comprising a polymeric material and magnetic particles disposed on the first coating layer, wherein the second coating layer is substantially free of the biologically active material; and (b) an electromagnetic energy source or a mechanical vibrational energy source for facilitating the delivery of the biologically active material.” Claim 27 of ublished U.S. patent application US 2004/0030379 claims: “27. A method for making a medical device for delivering a biologically active material to a patient comprising: (a) providing a medical device that is insertable into the body of the patient which comprises a surface; (b) disposing a first coating layer comprising a biologically active material on at least a portion of the surface; and (c) disposing a second coating layer comprising a polymeric material and plurality of magnetic particles on the first coating layer, wherein the second coating layer is substantially free of the biologically active material.”

In each of the medical device of claim 1 of published U.S. patent application US 2004/0030379, the system for deliverying a biologically active material to a patient of claim 15 of ublished U.S. patent application US 2004/0030379, and the method for making a medical device for deliverying a biologically active material of claim 27 of published U.S. patent application US 2004/0030379, the “magnetic particles” described in such claims can be advantageously replaced by the nanomagentic particles described in this specification.

“As was disclosed at page 1 of Published U.S. patent application US 2004/0030379, “The present invention generally relates to medical devices capable of providing on-demand delivery of biologically active material to a patient. In particular, the invention is directed to medical devices comprising a biologically active material, which is released from the device when the biologically active material is needed by the patient. The biologically active material is released when the patient is exposed to an energy source, such as electromagnetic energy or mechanical vibrational energy. When electromagnetic energy is used the medical device should also comprise magnetic particles that facilitate the release of the biologically active material.”

As is also disclosed at page 1 of published U.S. patent application US 2004/0030379, “In order to treat a variety of medical conditions, insertable or implantable medical devices having a coating for release of a biologically active material have been used. For example, various types of drug-coated stents have been used for localized delivery of drugs to a body lumen. See U.S. Pat. No. 6,099,562 to Ding et al. Such stents have been used to prevent, inter alia, the occurrence of restenosis after balloon angioplasty. However, delivery of the biologically active material to the body tissue immediately after insertion or implantation of the stent may not be needed or desired. For instance, it may be more desirable to wait until restenosis occurs or begins to occur in a body lumen that has been stented with a drug-coated stent before the drug is released. Therefore, there is a need for implantable medical devices that can provide on-demand delivery of biologically active materials when such materials are required by the patient after implantation of the medical device. Also needed is a non-invasive method to facilitate or modulate the delivery of the biologically active material from the medical device after implantation.”

As is also disclosed at page 1 of published U.S. patent application US 2004/0030379, “These and other objectives are accomplished by the present invention. To achieve these objectives, we have invented an insertable medical device that permits on-demand delivery of a biologically active material from the medical device when it is implanted in a patient. The release of the biologically active material is modulated and/or facilitated by the application of an extracorporal or external energy source, such as an electromagnetic energy source or a mechanical vibrational energy source. More specifically, the medical device, that is insertable into the body of a patient, comprises a surface and a first coating layer disposed on at least a portion of the surface. The first coating layer comprises a biologically active material. A second coating layer is disposed over the first coating layer and comprises magnetic particles and a polymeric material. The second coating layer is substantially free of the biologically active material, and preferably free of any biologically active material. When the patient is exposed to an extracorporal electromagnetic energy source, the release of the biologically active material from the coated medical device is facilitated. In this way, the biologically active material can be delivered to the patient only when he or she requires such material.”

In one preferred embodiment of published U.S. patent application US 2004/0030379, “The present medical device of the present invention can provide a desired release profile of a biologically active material. The desired release profile can be achieved because the medical device is coated with a first coating layer comprising a biologically active material and a second coating layer comprising magnetic particles that overlies or covers the first coating layer. The second coating layer is substantially free of a biologically active material so that the biologically active material is not exposed and is protected during implantation and prior to release into the body lumen of a patient. Because the second coating layer is substantially free of any biologically active material, there can be a higher concentration of magnetic particles in the second coating layer than if there were a biologically active material in the second coating layer. In addition, when the magnetic particles in the second coating layer are exposed to an energy source and move out of the second layer, the biologically active material is not immediately released. Instead, there is a controlled release of the biologically active material because the biologically active material migrates from the first coating layer and through the second coating layer before being delivered to a body lumen of a patient.” It is in this embodiment in which the substitution of applicants' nanomagnetic particles can improve the properties of the device of published U.S. patent application US 2004/0030379. These nanomagnetic particles have improved magnetic and imageability properties.

As is also disclosed in published U.S. patent application US 2004/0030379, “The system of the present invention comprises (1) a medical device having a coating containing a biologically active material, and (2) a source of electromagnetic energy or a source for generating an electromagnetic field. The present invention can facilitate and/or modulate the delivery of the biologically active material from the medical device. The release of the biologically active material from the medical device is facilitated or modulated by the electromagnetic energy source or field. To utilize the system of the present invention, the practitioner may implant the coated medical device using regular procedures. After implantation, the patient is exposed to an extracorporal or external electromagnetic energy source or field to facilitate the release of the biologically active material from the medical device. The delivery of the biologically active material is on-demand, i.e., the material is not delivered or released from the medical device until a practitioner determines that the patient is in need of the biologically active material. The coating of the medical device of the present invention further comprises particles comprising a magnetic material, i.e., magnetic particles. An example of the medical device of the present invention is illustrated in FIG. 1. The medical device is a stent 10 which is comprised of wire-like coated struts 20.”

As is also disclosed in published U.S. patent application US 2004/0030379, “An embodiment of the medical device of the present invention is illustrated in FIG. 2A. FIG. 2A shows a cross-sectional view of a coated strut of a stent. The coated strut 20 comprises a strut 25 having a surface 30. The coated strut 20 has a coating that comprises a first coating layer 40 that contains a biologically active material 45. Preferably, this coating layer also contains a polymeric material. A second coating layer 50 comprising magnetic particles 55 is disposed over the first coating layer 40. This second coating layer can also include a polymeric material. A third coating layer or sealing layer 60 is disposed on top of the second coating layer 50. FIG. 2B illustrates the effect of exposing a patient, who is implanted with a stent having struts shown in FIG. 2A, to an electromagnetic energy source or field 90. When such a field is applied, the magnetic particles 55 move out of the second coating layer 50 as shown by the upward arrow 110. This movement disrupts the sealing layer 60 and forms channels 100 in the sealing layer 60. The size of the channels 100 formed generally depends on the size of the magnetic particles 55 used. The biologically active material 45 can then be released from the coating through the disrupted sealing layer 60 into the surrounding tissue 120. The duration of exposure to the field and the strength of the electromagnetic field 90 determine the rate of delivery of the biologically active material 45.”

As is also disclosed in published U.S. patent application US 2004/0030379, “FIG. 3A shows another specific embodiment of a coated stent strut 20. The coating comprises a first coating layer 40 comprising a biologically active material 45 and preferably a polymeric material disposed over the surface 30 of the strut 25. A second coating layer or sealing layer 70 comprising magnetic particles 55 and a polymeric material is disposed on top of the first coating layer 40. FIG. 3B illustrates the effect of exposing a patient who is implanted with a stent having struts shown in FIG. 3A, to an electromagnetic field 90. When such a field is applied, the magnetic particles 55 move through the sealing layer 70 as shown by the upward arrow 110 and created channels 100 in the sealing layer 70. The biologically active material 45 in the underlying first coating layer 40 is allowed to travel through the channels 100 in the sealing layer 70 and be released to the surrounding tissue 120. Since the biologically active material 45 is in a separate first coating layer 40 and must migrate through the second coating layer or the sealing layer 70, the release of the biologically active material 45 is controlled after formation of the channels 100.”

As is also disclosed in published U.S. patent application US 2004/0030379, “FIG. 4A shows another embodiment of a coated stent strut. The coating comprises a coating layer 80 comprising a biologically active material 45, magnetic particles 55 and a polymeric material. FIG. 4B illustrates the effect of exposing a patient, who is implanted with a stent having struts shown in FIG. 4A to an electromagnetic field 90. The field is applied, the magnetic particles 55 move through the layer 80 as shown by the arrow 110 and create channels in the coating layer 80. The biologically active material 45 can then be released to the surrounding tissue 120.”

As is also disclosed in published U.S. patent application US 2004/0030379, “In another embodiment, the medical device of the present invention may be a stent having struts coated with a coating comprising more than one coating layer containing a magnetic material. FIG. 5 illustrates such a coated strut 20. The coating comprises a first coating layer 40 containing a polymeric material and a biologically active material 45 which is disposed on the surface 30 of a strut 25. A second coating layer 50 comprising a polymeric material and magnetic particles 55 is disposed over the first coating layer 40. A third coating layer 44 comprising a polymeric material and a biologically active material 45 is disposed over the second coating layer 50. A fourth coating layer 54 comprising a polymeric material and magnetic particles 55 is disposed over this third layer 44. Finally a sealing layer 60 of a polymeric material is disposed over the fourth coating layer 54. The permeability of the coating layers may be different from layer to layer so that the release of the biologically active material from each layer can differ. Also, the magnetic susceptibility of the magnetic particles may differ from layer to layer. The magnetic susceptibility may be varied using different concentrations or percentages of magnetic particles in the coating layers. The magnetic susceptibility of the magnetic particles may also be varied by changing the size and type of material used for the magnetic particles. When the magnetic susceptibility of the magnetic particles differs from layer to layer, different excitation intensity and/or frequency are required to activate the magnetic particles in each layer.”

As is also disclosed in published U.S. patent application US 2004/0030379, “Furthermore, the magnetic particles can be coated with a biologically active material and then incorporated into a coating for the medical device. In a preferred embodiment, the biologically active material is a nucleic acid molecule. The nucleic acid coated magnetic particles may be formed by painting, dipping, or spraying the magnetic particles with a solution comprising the nucleic acid. The nucleic acid molecules may adhere to the magnetic particles via adsorption. Also the nucleic acid molecules may be linked to the magnetic particles chemically, via linking agents, covalent bonds, or chemical groups that have affinity for charged molecules. Application of an external electromagnetic field can cause the adhesion between the biologically active material and the magnetic particle to break, thereby allowing for release of the biologically active material.”

As is also disclosed in published U.S. patent application US 2004/0030379, “In another specific embodiment, the magnetic particles may be molded into or coated onto a non-metallic medical device, including a bio-absorb able medical device. The magnetic properties of the magnetic particles allow the non-metallic implant to be extracorporally imaged, vibrated, or moved. In specific embodiments, the magnetic particles are painted, dipped or sprayed onto the outer surface of the device. The magnetic particles may also be suspended in a curable coating, such as a UV curable epoxy, or they may be electrostatically sprayed onto the medical device and subsequently coated with a UV or heat curable polymeric material.”

As is also disclosed in published U.S. patent application US 2004/0030379, “Furthermore, in certain embodiments, the movement of the magnetic particles that occurs when the patient implanted with the coated device is exposed to an external electromagnetic field, can release mechanical energy into the surrounding tissue in which the medical device is implanted and trigger histamine production by the surrounding tissues. The histamine has a protective effect in preventing the formation of scar tissues in the vicinity at which the medical device is implanted.”

As is also disclosed in published U.S. patent application US 2004/0030379, “Also the application of the external electromagnetic field can activate the biologically active material in the coating of the medical device. A biologically active material that may be used in this embodiment may be a thermally sensitive substance that is coupled to nitric oxide, e.g., nitric oxide adducts, which prevent and/or treat Adverse effects associated with use of a medical device in a patient, such as restenosis and damaged blood vessel surface. The nitric oxide is attached to a carrier molecule and suspended in the polymer of the coating, but it is only biologically active after a bond breaks releasing the smaller nitric oxide molecule in the polymer and eluting into the surrounding tissue. Typical nitric oxide adducts include nitroglycerin, sodium nitroprusside, S-nitroso-proteins, S-nitroso-thiols, long carbon-chain lipophilic S-nitrosothiols, S-nitrosodithiols, iron-nitrosyl compounds, thionitrates, thionitrites, sydnonimines, furoxans, organic nitrates, and nitrosated amino acids, preferably mono- or poly-nitrosylated proteins, particularly polynitrosated albumin or polymers or aggregates thereof. The albumin is preferably human or bovine, including humanized bovine serum albumin. Such nitric oxide adducts are disclosed in U.S. Pat. No. 6,087,479 to Stamler et al. which is incorporated herein by reference.”

As is also disclosed in published U.S. patent application US 2004/0030379, “Moreover, the application of electromagnetic field may effect a chemical change in the polymer coating thereby allowing for faster release of the biologically active material from the coating.”

As is also disclosed in published U.S. patent application US 2004/0030379, “Another embodiment of the present invention is a system for delivering a biologically active material to a body of a patient that comprises a mechanical vibrational energy source and an insertable medical device comprising a coating containing the biologically active material. The coating can optionally contain magnetic particles. After the device is implanted in a patient, the biologically active material can be delivered to the patient on-demand or when the material is needed by the patient. To deliver the biologically active material, the patient is exposed to an extracorporal or external mechanical vibrational energy source. The mechanical vibrational energy source includes various sources which cause vibration such as sonic or ultrasonic energy. Exposure to such energy source causes disruption in the coating that allows for the biologically active material to be released from the coating and delivered to body tissue.”

As is also disclosed in published U.S. patent application US 2004/0030379, “Moreover, in certain embodiments, the biologically active material contained in the coating of the medical device is in a modified form. The modified biologically active material has a chemical moiety bound to the biologically active material. The chemical bond between the moiety and the biologically active material is broken by the mechanical vibrational energy. Since the biologically active material is generally smaller than the modified biologically active material, it is more easily released from the coating. Examples of such modified biologically active materials include the nitric oxide adducts described above.”

As is also disclosed in published U.S. patent application US 2004/0030379, “In another embodiment, the coating comprises at least a coating layer containing a polymeric material whose structural properties are changed by mechanical vibrational energy. Such change facilitates release of the biologically active material which is contained in the same coating layer or another coating layer.”

As is also disclosed in published U.S. patent application US 2004/0030379, “The medical devices of the present invention are insertable into the body of a patient. Namely, at least a portion of such medical devices may be temporarily inserted into or semi-permanently or permanently implanted in the body of a patient. Preferably, the medical devices of the present invention comprise a tubular portion which is insertable into the body of a patient. The tubular portion of the medical device need not to be completely cylindrical. For instance, the cross-section of the tubular portion can be any shape, such as rectangle, a triangle, etc., not just a circle.”

As is also disclosed in published U.S. patent application US 2004/0030379, “The medical devices suitable for the present invention include, but are not limited to, stents, surgical staples, catheters, such as central venous catheters and arterial catheters, guidewires, balloons, filters (e.g., vena cava filters), cannulas, cardiac pacemaker leads or lead tips, cardiac defibrillator leads or lead tips, implantable vascular access ports, stent grafts, vascular grafts or other grafts, interluminal paving system, intra-aortic balloon pumps, heart valves, cardiovascular sutures, total artificial hearts and ventricular assist pumps.”

As is also disclosed in published U.S. patent application US 2004/0030379, “Medical devices which are particularly suitable for the present invention include any kind of stent for medical purposes, which are known to the skilled artisan. Suitable stents include, for example, vascular stents such as self-expanding stents and balloon expandable stents. Examples of self-expanding stents useful in the present invention are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 4,733,665 issued to Palmaz, U.S. Pat. No. 4,800,882 issued to Gianturco, U.S. Pat. No. 4,886,062 issued to Wiktor and U.S. Pat. No. 5,449,373 issued to Pinchasik et al. A bifurcated stent is also included among the medical devices suitable for the present invention.”

As is also disclosed in published U.S. patent application US 2004/0030379, “The medical devices suitable for the present invention may be fabricated from polymeric and/or metallic materials. Examples of such polymeric materials include polyurethane and its copolymers, silicone and its copolymers, ethylene vinyl-acetate, poly(ethylene terephthalate), thermoplastic elastomer, polyvinyl chloride, polyolephines, cellulosics, polyamides, polyesters, polysulfones, polytetrafluoroethylenes, acrylonitrile butadiene styrene copolymers, acrylics, polyactic acid, polyclycolic acid, polycaprolactone, polyacetal, poly(lactic acid), polylactic acid-polyethylene oxide copolymers, polycarbonate cellulose, collagen and chitins. Examples of suitable metallic materials include metals and alloys based on titanium (e.g., nitinol, nickel titanium alloys, thermo-memory alloy materials), stainless steel, platinum, tantalum, nickel-chrome, certain cobalt alloys including cobalt-chromium-nickel alloys (e.g., Elgiloy® and Phynox®) and gold/platinum alloy. Metallic materials also include clad composite filaments, such as those disclosed in WO 94/16646.”

As is also disclosed in published U.S. patent application US 2004/0030379, “In the instant specification, the term “magnetic particles” means particles comprising a magnetic material. Magnetic materials include ferromagnetic substances, i.e., substances which exhibit good magnetic susceptibility, such as ferrous substance including iron oxide steel, stainless steel; paramagnetic substances, such as aluminum, which have unpaired electrons and are attracted into a magnetic field; diamagnetic substances, such as gold, wherein all electrons are paired and are slightly repelled by the electromagnetic field. Preferably, the magnetic particles used for the present invention comprise a ferromagnetic substance. However, magnetic particles comprising paramagnetic or diamagnetic substances are particularly useful for imaging the medical device in a patient's body, for example, using magnetic resonance imaging (“MRI”) because the strong magnetic field in MRI would not negatively affect the particles but would enable or enhance the ability of MRI to detect them.” It is these “magnetic particles” of published U.S. patent application US 2004/0030379 which the nanomagnetic particles of applicants' invention replace. In one embodiment, the properties of such nanomagentic particles are chosen so that the magnetic susceptibility of the implanted medical device, when it is in contact with biological material, has a magnetic susceptibility of plus or minus 1×10⁻³ cgs.

As is also disclosed in published U.S. patent application US 2004/0030379, “The magnetic particles may be capsules made of non-magnetic substance, such as silica, encapsulating a magnetic substance or particles made of a mixture of a nonmagnetic substance and a magnetic substance. Also, the magnetic particles may be coated with a polymeric material to reduce any undesirable effects that may be caused by the corrosive nature of the magnetic substance. In another embodiment, ferrous loaded polymers are incorporated into the coating instead of magnetic particles. Examples of the ferrous loaded polymers include iron dextran.”

As is also disclosed in published U.S. patent application US 2004/0030379, “The average size of the particles is normally within the range from about 0.01 μm to about 10 μm. However, the average particle size may be any other suitable range such as from about 0.01 μm to about 50 μm. The sizes should be determined based on various factors including a thickness of the coating layer in which the particles are contained or by which the particles are covered, and desired release rate of the biologically active material. Also, when the biologically active material to be released from the medical device has comparatively greater size, i.e., cells or other large size genetic materials, the magnetic particles of greater size should be chosen. Suitable particles are not limited to any particular shape.”

As is also disclosed in published U.S. patent application US 2004/0030379, “Magnetic particles useful for the present invention, such as magnetic iron oxide particles (mean particle diameter 200 nm, density 5.35 g/cm3 and magnetization 30 emu/g) and magnetic silica particles, Sicaster-M™ (mean particle diameter 800-1500 nm, density 2.5 g/cm3 and magnetization ^{˜}4.0 emu/g) are commercially available, for example, from Micromod Partikeltechnologie.”

As is also disclosed in published U.S. patent application US 2004/0030379, “The concentration of the magnetic particles in a coating should be determined based on various factors including the size of the particles and desired release rate of the biologically active material. Normally, the concentration of the magnetic particles in a coating ranges from about 2% to about 20%.”

As is also disclosed in published U.S. patent application US 2004/0030379, “The term “biologically active material” encompasses therapeutic agents, such as drugs, and also genetic materials and biological materials. The genetic materials mean DNA or RNA, including, without limitation, of DNA/RNA encoding a useful protein stated below, anti-sense DNA/RNA, intended to be inserted into a human body including viral vectors and non-viral vectors. Examples of DNA suitable for the present invention include DNA encoding anti-sense RNA tRNA or rRNA to replace defective or deficient endogenous molecules, angiogenic factors including growth factors, such as acidic and basic fibroblast growth factors, vascular endothelial growth factor, epidermal growth factor, transforming growth factor α and β, platelet-derived endothelial growth factor, plateletderived growth factor, tumor necrosis factor α, hepatocyte growth factor and insulin like growth factor cell cycle inhibitors including CD inhibitors, thymidine kinase (“TK”) and other agents useful for interfering with cell proliferation, and the family of bone morphogenic proteins (“BMP's”) as explained below. Viral vectors include adenoviruses, gutted adenoviruses, adeno-associated virus, retroviruses, alpha virus (Semliki Forest, Sindbis, etc.), lentiviruses, herpes simplex virus, ex vivo modified cells (e.g., stem cells, fibroblasts, myoblasts, satellite cells, pericytes, cardiomyocytes, sketetal myocytes, macrophage), replication competent viruses (e.g., ONYX-015), and hybrid vectors. Non-viral vectors include artificial chromosomes and mini-chromosomes, plasmid DNA vectors (e.g., pCOR), cationic polymers (e.g., polyethyleneimine, polyethyleneimine (PEI)) graft copolymers (e.g., polyether-PEI and polyethylene oxide-PEI), neutral polymers PVP, SP1017 (SUPRATEK), lipids or lipoplexes, nanoparticles and microparticles with and without targeting sequences such as the protein transduction domain (PTD).”

As is also disclosed in published U.S. patent application US 2004/0030379, “The biological materials include cells, yeasts, bacteria, proteins, peptides, cytokines and hormones. Examples for peptides and proteins include growth factors (FGF, FGF-1, FGF-2, VEGF, Endotherial Mitogenic Growth Factors, and epidermal growth factors, transforming growth factor α and β, platelet derived endothelial growth factor, platelet derived growth factor, tumor necrosis factor α, hepatocyte growth factor and insulin like growth factor), transcription factors, proteinkinases, CD inhibitors, thymidine kinase, and bone morphogenic proteins (BMP's), such as BMP-2, BMP-3, BMP-4, BMP-5, BMP-6 (Vgr-1), BMP-7 (OP-1), BMP-8. BMP-9, BMP-10, BMP-11, BMP-12, BMP-13, BMP-14, BMP-15, and BMP-16. Currently preferred BMP's are BMP-2, BMP-3, BMP-4, BMP-5, BMP-6, BMP-7. Alternatively or in addition, molecules capable of inducing an upstream or downstream effect of a BMP can be provided. Such molecules include any of the “hedgehog” proteins, or the DNA's encoding them. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include whole bone marrow, bone marrow derived mono-nuclear cells, progenitor cells (e.g., endothelial progentitor cells) stem cells (e.g., mesenchymal, hematopoietic, neuronal), pluripotent stem cells, fibroblasts, macrophage, and satellite cells.”

As is also disclosed in published U.S. patent application US 2004/0030379, “Biologically active material also includes non-genetic therapeutic agents, such as: anti-thrombogenic agents such as heparin, heparin derivatives, urokinase, and PPack (dextrophenylalanine proline arginine chloromethylketone); anti-proliferative agents such as enoxaprin, angiopeptin, or monoclonal antibodies capable of blocking smooth muscle cell proliferation, hirudin, and acetylsalicylic acid, amlodipine and doxazosin; anti-inflammatory agents such as glucocorticoids, betamethasone, dexarnethasone, prednisolone, corticosterone, budesonide, estrogen, sulfasalazine, and mesalamine; immunosuppressants such as sirolimus (RAPAMYCIN), tacrolimus, everolimus and dexamethasone, antineoplastic/antiproliferative/anti-miotic agents such as paclitaxel, 5-fluorouracil, cisplatin, vinblastine, vincristine, epothilones, methotrexate, azathioprine, halofuginone, adriamycin, actinomycin and mutamycin; cladribine; endostatin, angiostatin and thymidine kinase inhibitors, and its analogs or derivatives; anesthetic agents such as lidocaine, bupivacaine, and ropivacaine; anti-coagulants such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors and tick antiplatelet peptides; vascular cell growth promotors such as growth factors, Vascular Endothelial Growth Factors (FEGF, all types including VEGF-2), growth factor receptors, transcriptional activators, and translational promotors; vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; and agents which interfere with endogenous vasoactive mechanisms; anti-oxidants, such as probucol; antibiotic agents, such as penicillin, cefoxitin, oxacillin, tobranycin angiogenic substances, such as acidic and basic fibrobrast growth factors, estrogen including estradiol (E2), estriol (E3) and 17-Beta Estradiol; and drugs for heart failure, such as digoxin, beta-blockers, angiotensin-converting enzyme (ACE) inhibitors including captopril and enalopril.”

As is also disclosed in published U.S. patent application US 2004/0030379, “Also, the biologically active materials of the present invention include trans-retinoic acid and nitric oxide adducts. A biologically active material may be encapsulated in micro-capsules by the known methods.”

As is also disclosed in published U.S. patent application US 2004/0030379, “The coating compositions suitable for the present invention can be applied by any method to a surface of a medical device to form a coating. Examples of such methods are painting, spraying, dipping, rolling, electrostatic deposition and all modern chemical ways of immobilization of bio-molecules to surfaces.”

As is also disclosed in published U.S. patent application US 2004/0030379, “The coating composition used in the present invention may be a solution or a suspension of a polymeric material and/or a biologically active material and/or magnetic particles in an aqueous or organic solvent suitable for the medical device which is known to the skilled artisan. A slurry, wherein the solid portion of the suspension is comparatively large, can also be used as a coating composition for the present invention. Such coating composition may be applied to a surface, and the solvent may be evaporated, and optionally heat or ultraviolet (UV) cured.”

As is also disclosed in published U.S. patent application US 2004/0030379, “The solvents used to prepare coating compositions include ones which can dissolve the polymeric material into solution and do not alter or adversely impact the therapeutic properties of the biologically active material employed. For example, useful solvents for silicone include tetrahydrofuran (THF), chloroform, toluene, acetone, isooctane, 1,1,1-trichloroethane, dichloromethane, and mixture thereof.”

As is also disclosed in published U.S. patent application US 2004/0030379, “A coating of a medical device of the present invention may consist of various combinations of coating layers. For example, the first layer disposed over the surface of the medical device can contain a polymeric material and a first biologically active material. The second coating layer, that is disposed over the first coating layer, contains magnetic particles and optionally a polymeric material. The second coating layer protects the biologically active material in the first coating layer from exposure during implantation and prior to delivery. Preferably, the second coating layer is substantially free of a biologically active material.”

As is also disclosed in published U.S. patent application US 2004/0030379, “Another layer, i.e. sealing layer, which is free of magnetic particles, can be provided over the second coating layer. Further, there may be another coating layer containing a second biologically active material disposed over the second coating layer. The first and second biologically active materials may be identical or different. When the first and second biologically active material are identical, the concentration in each layer may be different. The layer containing the second biologically active material may be covered with yet another coating layer containing magnetic particles. The magnetic particles in two different layers may have an identical or a different average particle size and/or an identical or a different concentrations. The average particle size and concentration can be varied to obtain a desired release profile of the biologically active material. In addition, the skilled artisan can choose other combinations of those coating layers.”

As is also disclosed in published U.S. patent application US 2004/0030379, “Alternatively, the coating of a medical device of the present invention may comprise a layer containing both a biologically active material and magnetic particles. For example, the first coating layer may contain the biologically active material and magnetic particles, and the second coating layer may contain magnetic particles and be substantially free of a biologically active material. In such embodiment, the average particle size of the magnetic particles in the first coating layer may be different than the average particle size of the magnetic particles in the second coating layer. In addition, the concentration of the magnetic particles in the first coating layer may be different than the concentration of the magnetic particles in the second coating layer. Also, the magnetic susceptibility of the magnetic particles in the first coating layer may be different than the magnetic susceptibility of the magnetic particles in the second coating layer.”

As is also disclosed in published U.S. patent application US 2004/0030379, “The polymeric material should be a material that is biocompatible and avoids irritation to body tissue. Examples of the polymeric materials used in the coating composition of the present invention include, but not limited to, polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate, styrene-isobutylene copolymers and blends and copolymers thereof. Also, other examples of such polymers include polyurethane (BAYHDROL®, etc.) fibrin, collagen and derivatives thereof, polysaccharides such as celluloses, starches, dextrans, alginates and derivatives, hyaluronic acid, and squalene. Further examples of the polymeric materials used in the coating composition of the present invention include other polymers which can be used include ones that can be dissolved and cured or polymerized on the medical device or polymers having relatively low melting points that can be blended with biologically active materials. Additional suitable polymers include, thermoplastic elastomers in general, polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid-polyethylene oxide copolymers, EPDM (etylene-propylene-diene) rubbers, fluorosilicones, polyethylene glycol, polysaccharides, phospholipids, and combinations of the foregoing. Preferred is polyacrylic acid, available as HYDROPLUS® (Boston Scientific Corporation, Natick, Mass.), and described in U.S. Pat. No. 5,091,205, the disclosure of which is hereby incorporated herein by reference. In a most preferred embodiment of the invention, the polymer is a copolymer of polylactic acid and polycaprolactone.”

As is also disclosed in published U.S. patent application US 2004/0030379, “More preferably for medical devices which undergo mechanical challenges, e.g. expansion and contraction, the polymeric materials should be selected from elastomeric polymers such as silicones (e.g. polysiloxanes and substituted polysiloxanes), polyurethanes, thermoplastic elastomers, ethylene vinyl acetate copolymers, polyolefin elastomers, and EPDM rubbers. Because of the elastic nature of these polymers, the coating composition adheres better to the surface of the medical device when the device is subjected to forces, stress or mechanical challenge.”

As is also disclosed in published U.S. patent application US 2004/0030379, “The amount of the polymeric material present in the coatings can vary based on the application for the medical device. One skilled in the art is aware of how to determine the desired amount and type of polymeric material used in the coating. For example, the polymeric material in the first coating layer may be the same as or different than the polymeric material in the second coating layer. The thickness of the coating is not limited, but generally ranges from about 25 μm to about 0.5 mm. Preferably, the thickness is about 30 μm to 100 μm.”

As is also disclosed in published U.S. patent application US 2004/0030379, “An external electromagnetic source or field may be applied to the patient having an implanted coated medical device using any method known to skilled artisan. In the method of the present invention, the electromagnetic field is oscillated. Examples of devices which can be used for applying an electromagnetic field include a magnetic resonance imaging (“MRI”) apparatus. Generally, the magnetic field strength suitable is within the range of about 0.50 to about 5 Tesla (Webber per square meter). The duration of the application may be determined based on various factors including the strength of the magnetic field, the magnetic substance contained in the magnetic particles, the size of the particles, the material and thickness of the coating, the location of the particles within the coating, and desired releasing rate of the biologically active material.”

As is also disclosed in published U.S. patent application US 2004/0030379, “In an MRI system, an electromagnetic field is uniformly applied to an object under inspection. At the same time, a gradient magnetic field, superposing the electromagnetic field, is applied to the same. With the application of these electromagnetic fields, the object is applied with a selective excitation pulse of an electromagnetic wave with a resonance frequency which corresponds to the electromagnetic field of a specific atomic nucleus. As a result, a magnetic resonance (MR) is selectively excited. A signal generated is detected as an MR signal. See U.S. Pat. No. 4,115,730 to Mansfield, U.S. Pat. No. 4,297,637 to Crooks et al., and U.S. Pat. No. 4,845,430 to Nakagayashi. For the present invention, among the functions of the MRI apparatus, the function to create an electromagnetic field is useful for the present invention. The implanted medical device of the present can be located as usually done for MRI imaging, and then an electromagnetic field is created by the MRI apparatus to facilitate release of the biologically active material. The duration of the procedure depends on many factors, including the desired releasing rate and the location of the inserted medical device. One skilled in the art can determine the proper cycle of the electromagnetic field, proper intensity of the electromagnetic field, and time to be applied in each specific case based on experiments using an animal as a model.”

As is also disclosed in published U.S. patent application US 2004/0030379, “In addition, one skilled in the art can determine the excitation source frequency of the elecromagnetic energy source. For example, the electromagnetic field can have an excitation source frequency in the range of about 1 Hertz to about 300 kiloHertz. Also, the shape of the frequency can be of different types. For example, the frequency can be in the form of a square pulse, ramp, sawtooth, sine, triangle, or complex. Also, each form can have a varying duty cycle.”

As is also disclosed in published U.S. patent application US 2004/0030379, “The mechanical vibrational energy source includes various sources which cause vibration such as ultrasound energy. Examples of suitable ultrasound energy are disclosed in U.S. Pat. No. 6,001,069 to Tachibana et al. and U.S. Pat. No. 5,725,494 to Brisken, PCT publications WO00/16704, WO00/18468, WO00/00095, WO00/07508 and WO99/33391, which are all incorporated herein by reference. Strength and duration of the mechanical vibrational energy of the application may be determined based on various factors including the biologically active material contained in the coating, the thickness of the coating, structure of the coating and desired releasing rate of the biologically active material.”

As is also disclosed in published U.S. patent application US 2004/0030379, “Various methods and devices may be used in connection with the present invention. For example, U.S. Pat. No. 5,895,356 discloses a probe for transurethrally applying focused ultrasound energy to produce hyperthermal and thermotherapeutic effect in diseased tissue. U.S. Pat. No. 5,873,828 discloses a device having an ultrasonic vibrator with either a microwave or radio frequency probe. U.S. Pat. No. 6,056,735 discloses an ultrasonic treating device having a probe connected to a ultrasonic transducer and a holding means to clamp a tissue. Any of those methods and devices can be adapted for use in the method of the present invention.”

As is also disclosed in published U.S. patent application US 2004/0030379, “Ultrasound energy application can be conducted percutaneously through small skin incisions. An ultrasonic vibrator or probe can be inserted into a subject's body through a body lumen, such as blood vessels, bronchus, urethral tract, digestive tract, and vagina. However, an ultrasound probe can be appropriately modified, as known in the art, for subcutaneous application. The probe can be positioned closely to an outer surface of the patient body proximal to the inserted medical device.”

As is also disclosed in published U.S. patent application US 2004/0030379, “The duration of the procedure depends on many factors, including the desired releasing rate and the location of the inserted medical device. The procedure may be performed in a surgical suite where the patient can be monitored by imaging equipment. Also, a plurality of probes can be used simultaneously. One skilled in the art can determine the proper cycle of the ultrasound, proper intensity of the ultrasound, and time to be applied in each specific case based on experiments using an animal as a model.”

As is also disclosed in published U.S. patent application US 2004/0030379, “In addition, one skilled in the art can determine the excitation source frequency of the mechanical vibrational energy source. For example, the mechanical vibrational energy source can have an excitation source frequency in the range of about 1 Hertz to about 300 kiloHertz. Also, the shape of the frequency can be of different types. For example, the frequency can be in the form of a square pulse, ramp, sawtooth, sine, triangle, or complex. Also, each form can have a varying duty cycle.”

As is also disclosed in published U.S. patent application US 2004/0030379, “The present invention provides a method of treatment to reduce or prevent the degree of restenosis or hyperplasia after vascular intervention such as angioplasty, stenting, atherectomy and grafting. All forms of vascular intervention are contemplated by the invention, including, those for treating diseases of the cardiovascular and renal system. Such vascular intervention include, renal angioplasty, percutaneous coronary intervention (PCI), percutaneous transluminal coronary angioplasty (PTCA); carotid percutaneous transluminal angioplasty (PTA); coronary by-pass grafting, angioplasty with stent implantation, peripheral percutaneous transluminal intervention of the iliac, femoral or popliteal arteries, carotid and cranial vessels, surgical intervention using impregnated artificial grafts and the like. Furthermore, the system described in the present invention can be used for treating vessel walls, portal and hepatic veins, esophagus, intestine, ureters, urethra, intracerebrally, lumen, conduits, channels, canals, vessels, cavities, bile ducts, or any other duct or passageway in the human body, either in-born, built in or artificially made. It is understood that the present invention has application for both human and veterinary use.”

As is also disclosed in published U.S. patent application US 2004/0030379, “The present invention also provides a method of treatment of diseases and disorders involving cell overproliferation, cell migration, and enlargement. Diseases and disorders involving cell overproliferation that can be treated or prevented include but are not limited to malignancies, premalignant conditions (e.g., hyperplasia, metaplasia, dysplasia), benign tumors, hyperproliferative disorders, benign dysproliferative disorders, etc. that may or may not result from medical intervention. For a review of such disorders, see Fishman et al., 1985, Medicine, 2d Ed., J. B. Lippincott Co., Philadelphia.”

As is also disclosed in published U.S. patent application US 2004/0030379, “Whether a particular treatment of the invention is effective to treat restenosis or hyperplasia of a body lumen can be determined by any method known in the art, for example but not limited to, those methods described in this section. The safety and efficiency of the proposed method of treatment of a body lumen may be tested in the course of systematic medical and biological assays on animals, toxicological analyses for acute and systemic toxicity, histological studies and functional examinations, and clinical evaluation of patients having a variety of indications for restenosis or hyperplasia in a body lumen.”

As is also disclosed in published U.S. patent application US 2004/0030379, “The efficacy of the method of the present invention may be tested in appropriate animal models, and in human clinical trials, by any method known in the art. For example, the animal or human subject may be evaluated for any indicator of restenosis or hyperplasia in a body lumen that the method of the present invention is intended to treat. The efficacy of the method of the present invention for treatment of restenosis or hyperplasia can be assessed by measuring the size of a body lumen in the animal model or human subject at suitable time intervals before, during, or after treatment. Any change or absence of change in the size of the body lumen can be identified and correlated with the effect of the treatment on the subject. The size of the body lumen can be determined by any method known in the art, for example, but not limited to, angiography, ultrasound, fluoroscopy, magnetic resonance imaging, optical coherence tumography and histology.”

A preferred Container Coated with Magnetostrictive Material

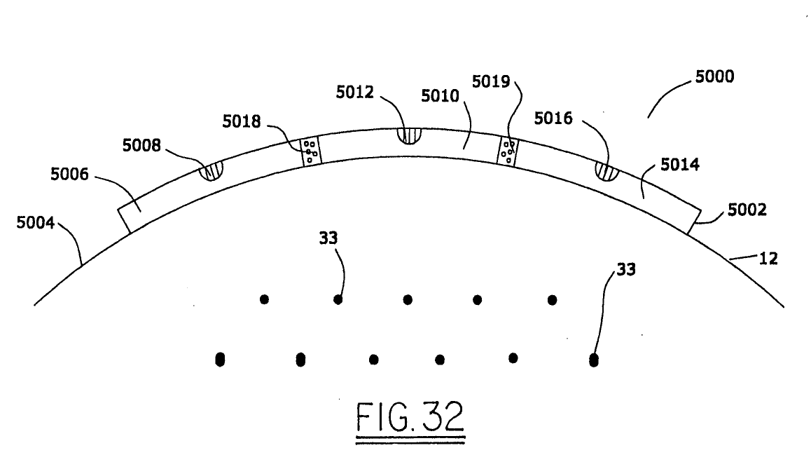

FIG. 32 is a partial view of a coated container 5000 comrised of a container 12 (see FIG. 1) over which is disposed a layer 5002 of material which changes its dimensions in response to an applied magnetic field. The material may be, e.g., magnetostrictivematerial, and/or it may be electrostrictive material. The direct current susceptibility of coated container 5000 is equal to the (mass of layer 5002)×(the susceptibility of layer 5002)+(the mass of container 12)×(the susceptibility of container 12).

As is known to those skilled in the art, magnetostriction is the dependence of the state of strain (dimensions) of a ferromagnetic sample on the direction and extent of its magnetization. Magnetostriction is discussed, e.g., at page 1106 of the McGraw-Hill Concise Encylopedia of Science and Technology, Third Edition (McGraw Hill Book Company, New York, N.Y., 1994), wherein it is defined as “The change of length of a ferromagnetic substance when it is magnetized. More generally, magnetostriction is the phenomenon that the state of strain of a ferromagnetic sample depends on the direction and extent of magnetization. The phenomenon has an important application is devices known as magnetostriction transducers.”

The phenomenon of magnetostriction has been widely discussed, and used in various devices, in the patent literature.

By way of illustration, and referring to U.S. Pat. No. 3,570,476 (the entire disclosure of which is hereby incorporated by reference into this specification), there is disclosed (in claim 4) “ . . . an element composed of material configured to be received into the interior of the artery and to be moved therealong and to establish mechanical vibrations in response to an applied signal . . . .” The material so used may be magnetostrictive material, or electrostrictive material. Thus, and as is discussed at columns 1 and 2 of U.S. Pat. No. 3,570,476, “ . . . the instrument of the invention may be inserted through an incision 10 in . . . the arm 12 of the patient. The magnetostrictive element 14 . . . is inserted through the incision 10, and into the interior of an artery . . . . The magnetostrictive element 14 may be excited in the manner to be described; or it may carry its own primary and secondary exciting coils, or other excitation means, which may be energized through electrical conductors in the wirelike element 18.

U.S. Pat. No. 3,570,476 also discloses that “ . . . The internal element 14 may be composed of a rod of magnetostrictive material, such as nickel, a ferrite formed, for example, of sintered oxides or iron, nickel, copper, or any other suitable magnetostrictive material. The rod, for example, may have a diameter of 1 millimeter. A damper 20 is mounted at one end of the rod 14. The damper 20 may be composed of any appropriate material, and should exhibit a relatively large mass with respect to the element 14, so that magnetostrictions set up in the element 14 result in a rapid movement of the end of the element remote from the damper 20. A biasing permanent magnet 21, formed of Alnico, ferrite or other appropriate permanent magnet material, should be interposed between the damper and the rod, as shown. In this way, the latter end of the element is caused to vibrate so as to dislodge and disperse cholesterol and other fatty deposits which have formed on the arterial wall . . . . The magnetostrictive effect is set up . . . by a secondary winding 22 which is wound about the periphery of the magnetostrictive element 14 and around the permanent magnet 21, and which has its ends short circuited, so that an appreciable current flows through the winding 22 when it is excited . . . . The core 26 has an airgap formed in it as shown . . . the core may be positioned over the arm of the patient so that the artery 16 being treated passes through the airgap even though the core 26 and primary winding are positioned externally of the patient. The primary winding 28 may be energized by an appropriate high frequency signal from a signal generator 30 of any suitable design. The frequency of the signal generated by the generator 30 may, for example, be in the range of from 25 kilohertz to 1 megahertz. Peak displacements of the order of 1 micrometer may be attained in the rod 22 when such parameters are used . . . . The embodiment illustrated in the drawing and described above is merely one aspect of the structural concept of the invention. For example, electrostrictive material such as barium titenate may be used, as will be described in conjunction with FIG. 5, and appropriate electrostatic fields produced by the voltage developed across an open secondary winding, rather than the current through a closed secondary winding as in the embodiment of FIG. 2. Moreover, a piezoelectric crystal may be used with plate contacts, and with the secondary winding connected to the plate contacts and establishing control voltages across the crystal. The piezoelectric and electrostrictive rods do not require biasing.”

By way of yet further illustration, and referring to U.S. Pat. No. 3,774,134 (the entire disclosure of which is hereby incorporated by reference into this specification), there is described (in claim 1 of this patent) “ . . . an extended length of anisotropic magnetic film plated wire having magnetostrictive properties . . . .”

In column 1 of U.S. Pat. No. 3,774,134, the phenomenon of magnetostriction is discussed. It is disclosed that: “The term magnetostriction is used to describe any dimensional change of a material which is associated with its magnetic behavior. Ferromagnetic bodies in particular are susceptible to dimensional changes as a result of changes in a magnetic field. In the following description, the phenomenon of interest is the converse, where change in stress on a magnetostrictive material induces a change in its magnetic behavior. These effects are described in detail in the copending application, Ser. No. 244,540 filed Apr. 17, 1972, and assigned to the same assignee as the present invention. In operation, an alternating current, sinusoidal or otherwise, is fed into the plated wire which generates an alternating magnetic field in the permalloy plating around the circumference of the wire. The alternating magnetic field sets the magnetization vector in the plated magnetic film into oscillation. This, in turn generates an alternating electromotive force in the substrate core of the wire, which core may be copper-beryllium. The voltage output or signal is alternating and constant in amplitude. Changes in the anisotropic constant of the film result in changes in the envelope of the signal amplitude. This appears as a modulation of a carrier similar in appearance to an amplitude modulation of a radio wave carrier. The transducer output is detected, filtered through a low pass-band filter, and amplified to produce an analogue signal.”

Referring again to FIG. 1, and to the preferred embodiment depicted therein, in one aspect of such embodiment the magentostrictive materials 5006, 5010, and 5014 do not have uniform properties. Means for varying the properties of one or more coatings of magnetorestrictive material are well known. Thus, and as is disclosed in claim 1 of such United States patent, the assembly of such patent comprises a “strain responsive anisotropic magnetostrictive thin film deposit on said substrate, said deposit being monotonically varied along the length of said wire in that at least one of the characteristics of the magnetic deposit is progressively modified thereby providing a controlled variation of relevant properties along the length of said wire, the voltage vs. strain response along the length of said wire increasing progressively from a relatively low level response at the source end of the wire to a relativelylarge response at the remote end of the wire.” The preparation of such a magnetostrictive thin film deposite is discussed at columns 3 and 4 of the patent, wherein it is disclosed that: “The anisotropic thin film permalloy possesses a gradient in magnetostriction along the wire. This is considered to be the most important single factor for the greater sensitivity of the line sensor at the far end. A magnetostrictive coefficient ratio at the “far-end” to the “source-end” in the order of 20:1 is feasible. The greater magnetostriction of the film at the far end causes the line sensor to possess greater sensitivity to strain at the distant location. Consequently, in spite of losses along the line, a significant signal may be transmitted over longer distances. The anisotropic permalloy thin film may also possess a plating thickness gradient along the wire. The thickness at the “far-end” must still be in the range of thin film so as to not adversely affect the desired magnetic properties of the film. A permissible range of plating thickness varies from about 5,000 Angstroms at the 37 source-end” to about 15,000 Angstroms at the ‘far-end.’ The anisotropic thin film may also possess a gradient in Hk along the wire for a single domain homogeneous ideal thin anisotropic film, Hk is defined as that field necessary to rotate the magnetization of the domain completely to the hard axis direction. An Hk ratio at the “far-end” to the “source-end” of 3:1 can be achieved without significantly altering the Hc/Hk ratio of the permalloy film. The lower values of Hk permit greater oscillatory response of the magnetization vector M to the drive current and also increase the strain induced rotational displacement of M generated by a stress signal. Since the high frequency drive current in the wire steadily decreases with distance along the wire, maintaining a gradient in Hk along the wire permits longer cable lengths. The Hk range is of the order of 3 oe. to 10 oe. These three items, the magnetostriction, the plating thickness and the Hk, singly or in any combination, preferentially increase the far end sensitivity of the line sensor. These changes or gradients are easily incorporated into the wire because plated wire fabrication is a continuous process and the desired gradients are incorporated into the plating by controlled changes in several process parameters. The plating thickness can be related to the duration and efficiency of the deposition process. Bath constituent concentrations and electric field density are also factors in controlling the amount of material deposited on the wire. Process parameters which directly control or influence the plating thickness include: wire speed through the plating line, plating current density, bath pH and temperature, electrolyte agitation around the wire in the plating cells and concentration of major and minor bath constituents. These parameters can be controlled individually or in various combinations to yield the desired gradient in film thickness along the wire. The magnetostriction coefficient, eta., which is related to the film composition, can be effectively varied by controlling such parameters as electrolyte flow and agitation, electric field distribution, bath pH, temperature and concentration of major and minor constituents, and the deposition potential.”

Referring again to U.S. Pat. No. 3,803,549, at column 2 of the patent it is disclosed haw it is dislosed how a variation in the amount of nickel and/or iron in the permalloy plating affects its magnetostrictive response. Thus, at such column 2, it is stated that: “permalloy plating is normally defined as an alloy of nickel and iron having approximately 80% nickel and 20% iron. Also at or about the approximate composition 80-20%, permalloy has a zero magnetostrictive response while an iron rich (Fe more than 20%) composition has a positive magnetostriction and a nickel rich (Ni more than 80%) composition of plating has a negative magnetostriction. In addition to selecting a positive or negative magnetostriction, the degree of magnetostriction may be selected by controlling the variance of the composition away from the zero magnetostrictive composition. If for purposes of description in the specification and claims the composition at or about 80-20% be accepted as the zero magnetostriction crossover, then as the composition is made iron rich out to 78-22% or thereabout, the positive magnetostriction increases as a factor of the variance from 80-20%, and as the composition is made increasingly nickel rich out to 82-18% or thereabout, the negative magnetostriction increases as a factor of the variance from the composition of 80-20%.”

By way of yet further illustration, and referring to U.S. Pat. No. 3,882,441 (the entire disclosure of which is hereby incorporated by reference into this specification), there is described (in claim 1) a “strain responsive anisotropic negatively magnetostrictive thin film deposit on said substrate, said film having a relatively low original average anisotropy field Hk of about 3 Oe., a dispersion in Hk which is low, and a coefficient of magnetostriction in the range from about −15,000 Oe. to about −20,000 Oe.” A detailed description of tis (and prior art) magnetostrictive thin film plated wires is presented at columns 2, 3, 4, 5, and 6 of such patent. At these columns, it is disclosed that: “Referring now to FIG. 1 . . . there is disclosed a cable 10 comprising a magnetostrictive thin film plated wire 11 having an insulating layer 16 within a conductive shield 13, the cable having a protective outer insulation 17 . . . . The anisotropic plated wire 11 may be, for example, a 10 mil diameter non-magnetic beryllium-copper substrate wire which has been plated with an anisotropic magnetostrictive permalloy (NiFe) film . . . . During deposition of the ferromagnetic film, a magnetic field is applied so that a preferred axis, called the easy axis, is obtained which is oriented circumferentially about the wire or with some other desired degree of skew. An applied circumferential field plus the D.C. plating current flowing in the wire during the film deposition causes a circumferential field in the wire film. In order to skew the field in the film, an external field is applied parallel to the wire during plating. Skew herein is defined as the angular measure by which the easy axis of the the field is displaced from a circumferential direction. The magnetization vector may lie along this line in the absence of external fields and strain on the wire, and makes a loop or helix of magnetic flux around the wire dependent upon the skew angle.”

As is also disclosed in U.S. Pat. No. 3,882,441, “In U.S. Pat. No. 3,657,641 to Kardashian . . . there is described in more detail anisotropic thin film plated wire of this nature. In that patent the permalloy film is described as being of approximate composition of 80% Ni and 20% Fe, which composition has a low or zero magnetostrictive effect. In the present invention which is a strain detector and which depends on the magnetostrictive response of the wire, it is desirable rather to select the various characteristics of the wire which enhance the magnetostrictive effect. Thus to be discussed below are several characteristics of the wire including those of reduced Hk, reduced Hk dispersion, magnetization skew angle β on the sensitivity of the wire, the effects of varying the coefficient of magnetostriction .eta. (i.e. the tensile strain sensitivity in Oersteds) and the effects of plating thickness.”

As is also disclosed in U.S. Pat. No. 3,882,441, “In accordance with the above, FIG. 2 shows schematically the contrasting slopes of Hk vs. tension curves for a nickel rich (i.e. negative magnetostrictive) wire in which Hk (induced) increases with increasing tension and an iron rich (i.e. positive magnetostriction) wire in which Hk (induced) decreases with increasing tension. A schematic representation of the Hk distribution of several wires is shown in FIG. 3; curve a showing a wire plating of high Hk dispersion and curve b showing a wire plating of low Hk dispersion which is much more suitable for line sensor application. It can be seen that the distribution of Hk in the high dispersion wire has significant components up to 30 Oe. and beyond. The desirable low Hk dispersion wire has an average Hk of about 3 Oe. The Hk distribution curve goes to zero at approximately 8 Oe. The contrast of the induced Hk vs. tension of three specific wires is shown in FIG. 4; the first of the wires is Fe. rich, has a moderate Hk (original)=5 Oe., a high Hk dispersion and a positive magnetostrictive coefficient .eta.=+16,000; the second of the wires is Ni. rich, has a moderate Hk (original)=7.3 Oe., a high Hk dispersion and a negative coefficient eta.=−12,000 Oe.; and the third of the wires is Ni. rich has a low Hk (original)=3 Oe., a low Hk dispersion and a negative coefficient .eta.=−24,000 Oe.”

As is also disclosed in U.S. Pat. No. 3,882,441, “At this point in the description, a discussion of the basic advantages of a strain sensitive wire for use as a line sensor in which the wire has negative magnetostriction in contrast to a wire having positive magnetostriction is in order. In a strain sensitive wire, the application of tension to one having negative magnetostriction causes its anisotropy field Hk to go up. The anisotropy field Hk is defined (for a single domain homogenous ideal thin anisotropic film) as that field necessary to rotate the magnetization vector of the domain completely to the hard axis direction. The lower values of Hk permit greater oscillatory response of the magnetization vector M to the drive current.”

As is also disclosed in U.S. Pat. No. 3,882,441, “If we assume for example, a relatively low Hk (original) of 3.0 Oe., as shown in FIG. 10 (curve of .eta.=−15,000), then the application of 100 gm. wt. causes the Hk (induced) to increase to approximately 5.0 Oe. and increasing the tension to 350 gm. wt. causes the Hk to increase to approximately 10.0 Oe. Thus when tensional force is applied to a negative magnetostrictive wire, the Hk goes up and therefore, the oscillations of the magnetization vector become smaller. This is desirable because no demagnetization of the wire occurs due to large strain signals. A strain sensing wire is thereby provided which is most sensitive under low DC loads (low strain) and relatively less sensitive under large DC loads.”

As is also disclosed in U.S. Pat. No. 3,882,441, “Now in contrast, the strain sensitive wire having positive magnetostriction is considered. The application of tension to a wire having positive magnetostriction causes the Hk to go down, which causes the oscillations of the magnetization vector to become larger (i.e. the sensitivity to increase). Because of the way positive magnetostrictive wire reacts to tension there are several disadvantages to its use as an extended length line sensor, in that on the one hand it is desired that Hk (original) be low so that the wire is sensitive under low loads signal levels, and on the other hand, the lowering Hk (induced) as DC strain increases allows the oscillations to increase and if the oscillations reach 90° the wire demagnetizes and becomes inoperable. Since in line sensor operation there is continually applied an alternating exciting current and thus an alternating field, if an increase in tension on the wire causes Hk to drop to a low value (0.5 Oe. for instance) the effect of the earth's field (<=0.5 Oe.) and the exciting field can exceed Hk (induced) and the wire will demagnetize. In most field uses tension is unpredictable, and an uncontrolable factor in the use of the line sensors is the magnitude of the stress signal caused by intrusion in the vicinity of the line. Therefore, there are limitations in the use of a positive magnetostrictive wire, and for a line sensor of extended length a negative magnetostriction wire according to this invention is to be preferred.”

U.S. Pat. No. 4,065,757, the entire disclosure of which is hereby incorporated by reference into this specification, also claims a wire substrate comprised of an anisotropic magnetostrictive magnetic film. Claim 1 of this patent describes: “a length of wire substrate having an anisotropic magnetostrictive magnetic film covering the wire substrate, the magnetic film having an easy axis of magnetization oriented helically around the wire, the helical magnetization direction being reversible by the application to said switch of external magnetic fields of predetermined magnitude to change the state of the magnetically actuated switch between a first state and a second state and to generate an electrical pulse in said wire substrate with each reversal between said states, said film covered substrate being known as a plated wire” The preparation of this magnetostrictive strain sensitive wire is discussed in columns 1 and 2 of such patent, wherein it is disclosed that: “In FIG. 1 an adjustable threshold thin-film plated wire magnetic switch is disclosed and comprises a length of plated wire 11 which is supported in tension by adjustable tension means 12 . . . . A section of the thin-film plated wire is shown in FIG. 3 in which the plating is magnetostrictive. The term magnetostriction is used to describe any dimensional change of a material which is associated with its magnetic behavior. Ferromagnetic bodies in particular are susceptible to dimensional changes, for instance, as a result of changes in temperature or a magnetic field. In the following description, the phenomenon of interest is the converse, where changes in strain on a magnetostrictive material induces a change in its magnetic behavior. Magnetostrictive strain sensitive wires typically comprise a permalloy plating on a conductive substrate wire such as copper-beryllium. A permalloy plating is normally defined as an alloy of nickel and iron. At or about the approximate composition 80% nickel and 20% iron permalloy has a zero magnetostrictive response while an iron rich (Fe more than 20 percent) composition has a positive magnetostriction and a nickel rich (Ni more than 80 percent) composition of plating has a negative magnetostriction. In addition to selecting a positive or negative magnetostriction, the degree of magnetostriction may be selected by controlling the variance of the composition away from the zero magnetostrictive composition. In the co-pending application of Lutes, mentioned above, the permalloy film is described as being of approximate composition of 80% Ni and 20% Fe, which composition has a low or zero magnetostrictive effect. In the present invention which depends on the magnetostrictive response of the wire, it is desirable rather to select a plated wire having negative magnetostriction.”

As is also disclosed in U.S. Pat. No. 4,065,757, “The anisotropic plated wire 11 may be, for example, a 10 mil diameter non-magnetic beryllium-copper substrate wire which has been plated with an anisotropic magnetostrictive permalloy (NiFe) film, a longitudinal-section of which is shown in FIG. 3. During deposition of the ferromagnetic film, a magnetic field is applied so that a preferred axis, called the easy axis, is obtained which is oriented helically about the wire. Pitch herein is defined as the angular measure by which the easy axis of the field is displaced from a circumferential direction. The magnetization vector may lie along this line in the absence of external fields on the wire, and makes a helix of magnetic flux around the wire dependent upon the pitch angle. The preferred pitch angle is in the range of about 15° to about 75°.”

As is also disclosed in U.S. Pat. No. 4,065,757, “A typical operational use of the magnetically actuated switch of this invention is as a proximity switch. The embodiment of the switch in a system as shown in FIG. 1 may be referred to as a single event switching mode. In this mode, the switch is set in one polarity (magnetization direction) prior to actuation. Broadly speaking, the switch is actuated by applying a magnetic field favoring the opposite polarity and having sufficient magnitude to exceed the coercive (threshold) value. This results in the generation of a single voltage pulse in a sense coil. Removal of the actuating field then results in resetting of the switch to the original polarity and another voltage pulse.”

As is also disclosed in U.S. Pat. No. 4,065,757, “The effect of an adjustment in the tension of plated wire 11 is shown in FIG. 5, where the induced coercive field H_{c} is plotted versus tension on the wire. In a strain sensitive wire, the application of tension to one having negative magnetostriction causes its coercive field H_{c} to go up. The coercive field H_{c} is defined (for a single domain homogeneous ideal thin anisotropic film) as that field which if increased slightly above the field at which domain wall motion begins, causes half the magnetization to be reversed . . . .”

U.S. Pat. No. 4,625,390, the entire disclosure of which is hereby incorporated by reference into this specification, discloses that one may affect the magnetic properties of a film by incorporating in such film “trivalent ions of negative magnetostriction constant.” Thus, and as is disclosed in column 3 of such patent, “it is another object of our invention to adjust the anisotropy field for a given content of bismuth by utilizing both growth-induced anisotropy by incorporation of one or more trivalent ions of negative magnetostriction constant in the (111) direction such as Gd, Sm, Tm, Dy, Ho, Er, Y, Yb or Lu into the film and by compression which is created by the lattice differential between film and substrate. The epitaxial film employed has a larger lattice constant that the substrate. The lattice differential may suitably be from about 0.005 A to about 0.06 A and is preferably greater than 0.017 A.” At columns 5-6 of this patent, it is disclosed that: “The use of ion implantation is a well-known procedure for altering magnetic anisotropy as evidenced by reference to U.S. Pat. No. 3,792,452 and the literature therein referred to. In the present invention the procedure of ion implantation is particularly well suited to effect the final adjustment of anisotropy field after the initial anisotropy field adjustment based on growth and strain induces changes. It should be added that excluding the specific contributions of both growth induced and strain induced lowering of anisotropy field, ion implantation alone would not serve to affect reduction of anisotropy field without adverse effect to the crystalline material. In considering the composites of the invention which are ion implanted, it should be noted that one of the essential parameters in the operation of a switchable magneto-optic device is that such device have an effective anisotropy field H*k=Hk −4 .pi. Ms, where Hk is the anisotropy field, 4 .pi. Ms is the demagnetizing field . . . . Generally speaking, H*k can most readily be brought into a suitable low operating range (300-400 Gauss) by ion implantation if the as grown film has a starting value of about 3000 gauss or less . . . . The Bi doped lanthanide garnet films of the present invention are suitably prepared by liquid phase epitaxy (LPE). These films are suitably deposited on (111) oriented gadolinium garnet substrates. In the process of the invention, very low temperatures are deliberately used for growth. By using growth temperatures in the vicinity of 700° C. lattice constants of the film, bigger than the substrate (compression) by as much as 0.06 A° at a thickness of 15 μm can be used. Low growth temperatures suppress the formation of dislocations because insufficient energy is provided to shift the lattice the full distance of its Burger's vector. Enough stress can then be selectively induced to effectively lower H*k from 10,000 Gauss anywhere down to zero. It has been discovered that this technique works regardless of whether the melt is leaded or unleaded or whatever other additives are in the melt.”

U.S. Pat. No. 4,650,281, the entire disclosure of which is hereby incorporated by reference into this specification, claims a magnetically sensitive optical fiber that comprises a central core of a magnetostrictive metal wire. The function of this magnetostrictive metal wire is described at columns 3-5 of the patent, wherein it is disclosed that: “Sensing arm 30 includes single-mode optical fiber 35 which contains magnetostrictive material. This fiber is sensitive to any magnetic field and particularly to one oriented substantially parallel to the longitudinal axis of this fiber. Specifically, the length of the fiber proportionally changes, e.g., elongates, in response and in proportion to any increase in the strength of the applied magnetic field H. This elongation changes the length of the optical path and, in so doing, imparts a phase change, i.e., phase modulates, the light propagating through optical fiber 35.”

As is also disclosed in U.S. Pat. No. 4,650,281, “referred dual core embodiment of a magnetically sensitive optical fiber . . . is shown in FIG. 2. As shown, central core 52 is comprised of a suitable magnetostrictive material, typified by iron, cobalt, nickel and various alloys and compounds thereof. Advantageously, these magnetostrictive materials are readily and inexpensively available in the form of wire of suitable gauge. Several glass cladding layers are concentrically clad to central core 52 to form an optical waveguide . . . . Magnetostrictive elongation of the core not only imparts a variable phase shift to the light propagating through the optical waveguide but also advantageously induces micro-bends onto the glass cladding layers which, in turn, advantageously decrease the amplitude of this light in the presence of a magnetic field. As the field strength increases, the number of applied micro-bends also increases. Hence, as the fiber elongates in the presence of a magnetic field, the resulting phase shift and amplitude loss both advantageously increase. Either or both of these effects can be used to sense magnetic field strength . . . .”

As is also disclosed in U.S. Pat. No. 4,650,281, “ . . . As a result, an optical waveguide (ring core and adjacent cladding layers) is concentrically formed around and coaxially oriented with the magnetostrictive wire which serves as core 52. As the fiber cools down to room temperature, the core contracts. Since the core material is chosen to have a higher thermal expansion coefficient value than any cladding layer(s), the contracting core places all the glass cladding layer(s) in compression . . . .”

As is also disclosed in U.S. Pat. No. 4,650,281, “Because magnetostrictive wire is readily available in a variety of different gauges which span a large range, the magnetostrictive material can be easily obtained with a relatively large diameter. By choosing a relatively large diameter wire, one can easily fabricate a magnetically sensitive optical fiber which will produce a significant change in length, e.g. elongation or contraction, in the glass cladding and, in turn, a large optical phase shift in the presence of a very weak magnetic field.”

As is also disclosed in U.S. Pat. No. 4,650,281, “For the embodiment shown in FIG. 2, the metallic core 52 may be illustratively comprised of nickel wire, which possesses a negative magnetostrictive coefficient (i.e. this wire contracts in the presence of a magnetic field applied parallel to its axis) having a diameter of approximately 40 μm (micrometers or microns) with optical ring core 54 having a thickness of approximately 5-10 μm . . . . Of course, it would be appreciated by those skilled in the art that both optical and electrical signals can be simultaneously transmitted through the inventive magnetostrictive optical fiber described hereinabove. Specifically, signals such as data, which require a wide bandwidth, can be transmitted as optical signals which propagate through the optical ring core. High current low bandwidth signals, such as power and/or control signals, can be advantageously transmitted in electrical form through magnetostrictive core 52.”

By way of yet further illustration, and referring to U.S. Pat. No. 4,803,501 (the entire disclosure of which is hereby incorporated by reference into this specification), the effect of the magnetostrictive coefficient is discussed. It is disclosed at columns 3-4 of this patent that: “the yoke-formed portion 1a of the preferred device can also be made of magneto-strictive material with opposite signs concerning the length variation when compared to the sign of length variation of the material forming the rod 3. If rod 3 is made of cobalt-iron alloy having a positive magneto-strictive coefficient causing an increase of the length under the influence of a magnetic field generated by the coil 4, said magnetic field also acting on portion la forming a close magnetic circuit with the rod 3 causes a decrease in length of the yoke-formed portion 1a, if the material thereof has a magneto-strictive coefficient, e.g. when using pure nickel for this purpose. The opposite relationship can also be achieved when forming the rod 3 of a material having a negative magneto-strictive coefficient, like nickel, whilst choosing a material of positive magneto-strictive coefficient for forming the portion la, e.g. cobalt-iron.”

U.S. Pat. No. 5,109,698, the entire disclosure of which is hereby incorporated by reference into this specification, illustrates a “borehole seismic transducer” that includes “an actuating means comprising a magnetostrictive driver.” In column 7 of this patent, and referring to FIG. 15 thereof, “Shell 152 and tension rod 158 are concentrically arranged, with shell 152 being made of a permeable metal. More specifically, shell 152 is made of a magnetostrictive material having a positive magnetostrictive coefficient, such as the alloy Alfer, which is 13% aluminum and 87% iron. Tension rod 158 is made from a magnetostrictive material having a negative magnetostrictive coefficient, such as nickel. Alternatively, the material for shell 152 and tension rod 158 can be negative for shell 152 and positive for tension rod 158. To avoid eddy current losses and optimize operating efficiency, tension rod 158 should be constructed using length oriented laminations.”

By way of further illustration, and referring to U.S. Pat. No. 5,129,789 (the entire disclosure of which is hereby incorporated by reference into this specification), one may use “mangetostrive tubing” to pump blood or other fluid. This patent claims: “A method of pumping useable blood comprising: connecting a useable blood inlet conduit and a useable blood outlet conduit to a length of magnetostrictive tubing having a longitudinal axis; keeping the length of magnetostrictive tubing under compression along its longitudinal axis; and imposing a pulsed electromagnetic field on the tubing to cause magnetostriction of the tubing and blood displacement in one direction.” At columns 3-4, the properties of some rare earth magnetostrictive materials. It is disclosed in these columns that: “The properties of rare earth magnetostrictive material are known in the art. See, for example, A. E. Clark, “Introduction to Highly Magnetostrictive Rare-Earth-Materials”, U.S. Navy Journal of Underwater Acoustics, 27, 109-125 (1977); A. E. Clark & D. N. Crowder, “High Temperature Magnetostriction of TbFe2 and Th.27 Oy.73 Fe2”, Trans. Mag., MAG-21, No. 5 (1985); R. W. Timme, “Magnetomechanical Characteristics of Terbium-Holmium-Iron Alloy,” J. Acoust. Soc. Am., 59, 459-464 (1976); “Proceedings of the First International Conference on Giant Magnetostrictive Alloys and Their Impact on Actuator and Sensor Technology,” Marbella Spain, Carl Tyren, Ed., Fotynova, Lund Sweden (March 1986). The properties of magnetostrictive materials are such that an imposition of a magnetic field upon the material causes it to change size. In fact, the material can be produced so that it can have directional expansion. Magnetostriction is defined as the change of length of a ferromagnetic substance when it is magnetized. More generally, magnetostriction is the phenomenon that the state of strain of a ferromagnetic sample depends on the direction and extent of magnetization.

U.S. Pat. No. 5,129,789 also discloses that “In the preferred embodiment of the invention, tubular section 12 is made from a material designated as ETREMA Terfenol-D®, which can be pre-processed to expand directionally in the presence of a magnetic field. This material is publicly available through Edge Technologies of Ames, Iowa. Terfenol is the binary rare earth iron alloy TbFe2. ETREMA Terfenol-D® is an alloy of the form Tbx Dy1-x Fe1.9-2. Directionally, solidified compositions can be produced by a freestand zone melt (FSZM) or a modified Bridgman (MB) method. In particular, in the presence of a magnetic field the tubular section 12 expands. As can be appreciated, lengthening of section 12 longitudinally results in shrinkage laterally; similarly to a rubber band which is stretched along its length. As can be understood by referring to FIG. 1, such expansion causes the distance 30 (between opposite open ends 14 and 16) to increase which in turn causes the distance between valves 22 and 24 to increase, as they are fixed to section 12. The distance designated by reference numeral 30, in the preferred embodiment shown in FIG. 1, changes approximately 1/1000th of an inch per inch of tubular section 12 at a 10 megacycle pulsing of coil 20. It is to be understood that section 12 would increase in length approximately twice as much as the bore 18 would be narrowed by the stretching expansion of tubular section 12. Thus, the interior volume of bore 18 increases upon magnetostriction and valves 22 and 24 move farther apart. This very high speed reciprocation results in the first one-way valve 22 opening and closing approximately at the same frequency. Because of these many but small movements of valve 22 along the fluid flow line, small amounts of fluid in inlet conduit 26 will pass through valve 22, each time it opens, into bore 18. As these small volumes of fluid enter bore 18, fluid pressure builds up and then causes a like amount of fluid to exit out of alternatingly opening and closing second one-way valve 24 at the outlet end 16 of tubular section 12. Thus, this structurally non-complex configuration operates at a high enough rate to pump fluid both through the pump itself as well as through a fluid circuit.”

As is also disclosed in U.S. Pat. No. 5,129,789, “FIG. 2 depicts schematically a specific application of pump 10 of FIG. 1. In this preferred embodiment, tubular section 12 is four inches long in its relaxed normal condition. The inside diameter of bore 18 is 14 millimeters. Coil 20 is an 8 ohm coil. Valves 22 and 24 are preferably Kolff tri-leaf polyurethane “Utah” valves (see FIGS. 7-10). In this embodiment, pump 10 can be placed inside or outside a patient and used as a total artificial heart, replacing the pumping function of the biological heart, or it can be used outside the patient as a ventricular assist device. Either way, inlet and outlet conduits 26 and 28 would be connected to the circulatory system of a patient, such as is known in the art.”

By way of yet further illustration, and referring to U.S. Pat. No. 5,570,251 (the entire disclosure of which is hereby incorporated by reference into this specification), there is disclosed a thin film magnetic device whose top and bottom surfaces have different magnetostrictive properties. This patent claims (in claim 1) “A thin film magnetic device, comprising: an underlying layer; a layer having a raised shape including an organic insulating layer, said layer having said raised shape provided directly or indirectly on said underlying layer; and a soft magnetic alloy thin film having a first end and a second end, said film covering said layer having said raised shape, said first end being fixedly joined to said underlying layer directly and said second end being fixedly joined to said underlying layer either directly or indirectly; said soft magnetic alloy thin film consisting of a top region, a bottom region, the top region having a height relative to the underlying layer, the bottom region having a height relative to the underlying layer, and the height of the top region being greater than the height of the bottom region for all points of the top region and the bottom region, and an intermediate region that is between said top region and said bottom region, wherein said film has a magnetostriction distribution varying from positive to negative magnetostriction values such that all of said top region has one of positive and negative magnetostriction, all of said bottom region has the other one of said positive and negative magnetostriction, and at least part of said intermediate region has zero magnetostriction.”

Referring again to U.S. Pat. No. 5,570,251, and to column 6 thereof, certain magnetostrictive alloys are discussed. It is disclosed that: “The soft magnetic thin film-forming magnetic substance used in this type of thin film magnetic device, that is, thin film magnetic head is generally selected from alloys containing a magnetic metal such as Co, Ni, and Fe as a major component and having uniaxial magnetic anisotropy, especially including a composition range having a magnetostriction value of zero. For example, it is known that for Permalloy or an NiFe alloy, approximately Ni-20 wt % Fe is a neutral composition having zero magnetostriction, and for a CoFe alloy, magnetostriction reaches zero at approximately Co-10 wt % Fe. Also useful are compositions of CoNiFe alloy along a zero magnetostriction line.”

As is also disclosed in U.S. Pat. No. 5,570,251, “An alloy has a magnetostriction value which shows a different behavior depending on crystallographic plane orientation, which implies that alloy samples of an identical composition exhibit different magnetostriction if their plane orientation is different. Further samples having an identical major component composition exhibit different magnetostriction depending on their crystal grain size and containment of a trace amount of impurity. Even in such a case, an alloy having composition regions exhibiting zero magnetostriction, positive magnetostriction and negative magnetostriction within a magnetic thin film is acceptable. What is important herein is not a composition, but a magnetostriction value.”

By way of yet further illustration, and referring to U.S. Pat. No. 5,633,092, a magnetostrictive material with two component parts is disclosed. This patent claims: “A magnetic material comprising: a first component layer having a first atomic structure; and a second component layer on said first component layer and having a second atomic structure which is non-homogeneous with said first atomic structure of said first component; a first surface of said first component layer being contiguous with a first surface of said second component layer; a lattice structure of said first component layer at least where said first surface of said first component layer is contiguous with said first surface of said second component layer being modified by said second component layer whereby a magnetostrictive property of said magnetic material is increased.”

In column 1 of U.S. Pat. No. 5,633,092, it is disclosed that: “Magnetostriction is the property which relates magnetic characteristics of a body of material to a change of the shape of the material. The property is seen in the change in size of bodies of certain materials when the magnetic environment changes or the change in magnetic characteristic when a force is applied to such a body to change its shape. Magnetostriction is a dimensionless quantity represented by the magnetostriction constant .lambda.S, relating magnetization and shape change and in the SI system of units useful values are some tens or hundreds of parts per million, particularly for use in sensors and transducers. For such uses a high magnetomechanical coupling factor is desirable. Also “soft” magnetic properties are generally preferred. While thin film amorphous alloys and magnetic multilayers individually provide some of the required properties there is still a strong need for a significant improvement in the properties available and for materials which exhibit a useful combination of such properties.”

The device of U.S. Pat. No. 5,633,092 is comprised of a magnetic material of at least two component parts arranged to have respective structures which mutually are not homogenous, the structure of one part cooperating with the structure of the other to assist the magnetostrictive behaviour of the material. At column 4 of the patent, some “prior art” multilayer materials having magnetic properties were discussed. It is disclosed in such column 4 that: “Various proposals for multilayer materials having magnetic properties have been made, for example de Wit, Witmer and Dime (Advanced Materials, Vol. 3 (1991) No. 7/8 pp 356 to 360) and Zeper, van Kesteren and Carcia (Advanced Materials, Vol. 3 (1991) No. 7/8 pp 397-399). In the first of these (de Wit, etc) a material with enhanced saturation magnetization and relative permeability but minimal magnetostriction, specifically for a video recorder read/write head, is described. To achieve this a very small grain size is sought for the magnetic material layer (iron, grain size below 10 nanometers) and to produce such a grain size the iron layers are around 10 nanometers thick. The layers are separated by thinner layers of another ferromagnetic material, specifically an iron/chromium/boron layer. This layer needs to be at least two nanometers thick to prevent the grains in one iron layer from linking with those in another iron layer by columnar growth and specifically epitaxial growth is not desired. In the second (Zeper et al) a magneto optical recording material is described, for enhanced recording density, which has adequate Kerr rotation and low enough Curie temperature while having a preferred magnetization direction perpendicular to the material layer. This preferred direction only occurs with cobalt/platinum or cobalt/palladium multilayers when the cobalt layers are less than some 0.8 to 1.2 nanometers. The thickness of the non-magnetic but magnetically polarisable platinum or palladium layer is set by the required balance of Kerr effect and Curie temperature and for platinum is about 0.9 nanometers with a 0.4 nanometer cobalt layer. The cobalt layer is about two atom layers thick so that the required magnetic anisotropy is not reduced by “bulk” atoms between the surface layers. In Zuberek, Szymczak, Krishnan and Tessier (Journal de Physique, Vol. 12, No. 9, Colloque C8, December 1988, pp 1761 to 1762) it is suggested that a “bilayer” of evaporated component materials depends for effectiveness on the thinness of a nickel layer. In Dirne, Tolboom, de Wit and Witmer (J. Magn. Magn. Mat., No. 83, (1990) pp 399 to 400) the possibility of interface mixing in Fe/Co multilayers is discussed and seen as a disadvantage.”

By way of yet further illustration, and referring to U.S. Pat. No. 5,717,330 (the entire disclosure of which is hereby incorporated by reference into this specification), a magnetostrictive linear displacement transducer is claimed. Some “prior art” transducers are discussed at columns 1-2 of the patent, wherein it is disclosed that: “A magnetostrictive effect has been utilized previously for linear displacement transducers. Examples are found in U.S. Pat. No. 3,898,555 to Tellerman and U.S. Pat. No. 5,017,867 to Dumais et al. A torsional motion sensor is used to detect torsional motion within the magnetostrictive wave guide tube induced by passage of an electrical pulse down a wire which interacts with a magnetic field of an adjacent magnet. The position of the magnet along the tube can thereby be determined. U.S. Pat. No. 5,198,761 to Hashimoto et al. discloses a stroke detector including a driving coil wound around a member with a large magnetostriction coefficient. A pulse input current to the coil causes magnetostriction phenomena on the magnetostriction line generating an ultrasonic wave. A detecting coil wound on the member induces a detection signal generated by reverse magnetostriction when the ultrasonic wave passes by the position of the magnet on the magnetostriction member. The prior art magnetostrictive transducers sold under the trademark TEMPOSONICS are adapted to fit within the piston rod of an hydraulic or pneumatic cylinder . . . . The device typically measures the position of four magnets which are oriented with their poles being spaced-apart radially with respect to the center line of the tube . . . .”

By way of yet futher illustration, and referring to U.S. Pat. No. 5,843,153 (the entire disclosure of which is hereby incorporated by reference into this specification), there is claimed “an implantable stylet . . . comprising: a first member; and a second member comprising a magnetostrictive material, wherein in the presence of a given magnetic field, the percent change in length of said second member is different than the percent change in length of said first member, further wherein said second member is fixedly coupled to said first member to cause said implantable stylet to curve under the influence of a given magnetic field.” This stylet is discussed, e.g., at column 4 of the patent, wherein it is disclosed that: “To give the stylet 20 the ability to be non-conformally curved, the stylet 20 uses at least two elements coupled together. At least one of these elements is capable of movement to produce a desired curvature in the stylet 20. FIGS. 3 and 4 illustrate one exemplary embodiment of the stylet 20. In this embodiment, the stylet 20 includes two material members 28 and 30 coupled together along at least a portion of the stylet 20. The member 28 is advantageously composed of a magnetostrictive material. The other member 30 is advantageously composed of a substrate material that is relatively non-magnetostrictive as compared with the member 28. Because magnetostrictive materials change length in response to the application of a magnetic field, the magnetostrictive member 28 will elongate in the presence of a suitable magnetic field. The magnetic field does not cause the substrate member 30 to change shape substantially, so it essentially retains its original length. Therefore, in the presence of a suitable magnetic field, the change in length of the magnetostrictive member 28 relative to the substrate member 30 produces a curvature in the stylet 20. It should also be noted that the substrate member 30 may be made of a magnetostrictive material that has a response opposite the magnetostrictive member 28 to achieve a relative change in length between the two members 28 and 30 in response to the presence of a suitable magnetic field. The type of deformation, e.g., elongation or contraction, depends upon the type of magnetostrictive material that is used. The magnitude of the change in length of the magnetostrictive member 28 depends upon the magnitude of the magnetic field applied axially to the magnetostrictive member 28 and upon the particular magnetostrictive material used. In this embodiment, the magnetostrictive member 28 is advantageously composed of TERFENOL-D available from Etrema Products, Inc., although other suitable types of magnetostrictive materials may also be used. The magnetostrictive material TERFENOL-D also exhibits inverse magnetostriction (known as the Villari effect), a phenomenon in which a change in magnetic induction occurs when a mechanical stress is applied along a specified direction to a material having magnetostrictive properties. These measurable changes in induction enable TERFENOL-D to be used in sensing applications (such as magnetotagging) where changes in stress occur. Consequently, the flexure of the device within the body can be sensed and used as a motion transducer for diagnostic purposes.”

As is also disclosed in U.S. Pat. No. 5,843,153, “Examples of suitable materials for the substrate member 30 may include titanium, aluminum, magnesium, and stainless steel. As with the materials used to form virtually all stylets, the material used to fashion the substrate member 30 advantageously has a relatively high flexibility to facilitate the large and frequent bending movements associated with in vivo insertions.”

By way of yet further illustration, and referring to U.S. Pat. No. 5,886,518, there is disclosed a nickel alloy magentostrictive wire. In particular, there is claimed in claim 1 “A magnetostrictive wire used in a displacement detection device together with a magnetostriction-generating magnet disposed close thereto and movable relative thereto, said wire substantially composed of 35 to 60 wt % Ni and the balance consisting of Fe and unavoidable impurities, cold-wire-drawn and then heat-treated and having a magnetostriction coefficient greater than that exhibited in an as-cold-wire-drawn state.”

At columns 1-2 of U.S. Pat. No. 5,886,518, some of the “prior art” mangetostrictive wires are discussed. It is disclosed that: “U.S. Pat. No. 3,173,131 discloses a magnetostrictive apparatus for displacement detection comprising a magnetostrictive wire, a permanent magnet movable along the wire, an oscillator means for applying a pulse current to the wire, and a receiver means disposed at a selected portion of the wire for receiving an ultrasonic wave or a magnetostriction signal generated in the wire in a portion close to the permanent magnet.”

As is also disclosed in U.S. Pat. No. 5,886,518, “Japanese Unexamined Patent Publication No. 2-183117 discloses a magnetostrictive wire made of an Elinvar alloy such as “NiSpanC” (trade name), which is a constant-modulus alloy having a modulus which does not vary with temperature. The temperature coefficient of the ultrasonic wave propagation speed of the Elinvar alloy can be reduced to 20 ppm/° C. or less by heat treatment or other processing conditions. In contrast, the measurement error due to temperature change in the detection circuitry generally ranges from 200 to 500 ppm/° C. Therefore, the variation of the ultrasonic wave propagation speed of the Elinvar alloy can practically be ignored. Thus, the Elinvar alloy is advantageously used as a material of magnetostrictive wire because of its small temperature coefficient of the resonance frequency and a stable magnetostriction transfer speed or twisting vibration speed which does not vary with temperature.”

As is also disclosed in U.S. Pat. No. 5,843,153, “On the other hand, the Elinvar alloy has a magnetostrictive coefficient (.lambda.) as small as about 5×10-6, and therefore, the displacement detection requires amplification at a high speed of response. Moreover, the magnetostrictive coefficient is slightly reduced at temperatures above 100° C., which also causes an error in the displacement detection.”

As is also disclosed in U.S. Pat. No. 5,843,153, “The object of the present invention is to provide a magnetostrictive wire which overcomes the drawbacks of the conventional Elinvar alloy wire so that not only the magnetostriction transfer speed but also the magnetostriction intensity do not vary with temperature and the magnetostriction coefficient is sufficiently great so that the displacement detection can be effected without the necessity of amplification at a high speed of response. To achieve the above object according to the present invention, there is provided a magnetostrictive wire for a displacement detection device together with a magnetostriction-generating magnet disposed close thereto and movable relative thereto, the wire substantially composed of 35 to 60 wt % Ni and the balance consisting of Fe and unavoidable impurities, cold-wire-drawn and then heat-treated and having a magnetostriction coefficient greater than that exhibited in an as-cold-wire-drawn state. The heat treatment is preferably carried out at a temperature of from 400° C. to 1100° C.”

As is also disclosed in U.S. Pat. No. 5,843,153, “The magnetostrictive wire according to the present invention is substantially composed of 35 to 60 wt % Ni and the balance consisting of Fe and unavoidable impurities. Namely, the present inventive alloy is based on a Permaloy-type alloy composed of 35 to 60 wt % Ni and the balance of Fe. It is conventionally well known that Permaloy-type alloys having compositions within that range have a large magnetostriction coefficient.”

As is also disclosed in U.S. Pat. No. 5,843,153, “Commercially available Permaloy-type alloys include a high Ni group including grade A (70 to 80 k wt % Ni) and grade C (70 to 80 Ni, with 4 to 14 wt % of one or two of Cu, Mo, Cr, and Nb) and a low Ni group including grade B (40 to 50 wt % Ni or 40 to 50 wt % Ni with 3 to 5 wt % one of Mo, Si, and Cu), grade D (35 to 40 wt % Ni) and grade E (45 to 55 wt % Ni). According to the present invention, the magnetostrictive wire is made of an alloy based on the Permaloy-type alloy of the low Ni group or grades B, D and E. The magnetostrictive wire of the present invention is typically made of an alloy composed of 50 wt % Ni and 50 wt % Fe.”

As is also disclosed in U.S. Pat. No. 5,843,153, “The magnetostrictive wire of the present invention may contain Mo, Si, and Cu, which are contained in some B grade Permaloy-type alloy, and may also contain a few wt % of other additives for improving permeability or corrosion resistance. The Elinvar-type alloys used for magnetostrictive wires include classes I, II, II, and IV, in which classes II, III, and IV do not contain Ni as a primary component, i.e., contain another element in an amount more than that of Ni, if Ni is contained. Class I purposely contains Cr as an additive to stabilize or render the temperature-caused variation of the shear modulus to be zero in the as-produced condition, although it contains Ni as a primary component, as typically exemplified by 36 wt % Ni-12 wt % Cr—Fe, which is called Elinvar. Some of the class I alloys further contain 0.5 to several wt % C, Ti, Mo, Si, Mn, Al, etc.”

By way of yet further illustration, and referring to U.S. Pat. No. 6,363,793 (the entire disclosure of which is hereby incorporated by reference into this specification), the “Villari effect” is discussed. As is disclosed, e.g., in column 3 of the patent, “The seat weight sensor of the present invention operates by utilizing the principal that the magnetic permeability of certain materials varies under the application of mechanical stress applied to the material. This principal is known as the Villari effect. More specifically, the Villari or “inverse Joule magnetoelastic” effect was discovered and studied by Joule and Villari in the mid 1800's. The Villari effect phenomenon occurs in ferromagnetic materials and is characterized by a change in the magnetic permeability of the material when subjected to stress. That is, the ability to magnetize the material depends upon the level of stress applied to the material. The Villari effect is closely related to the magnetostriction phenomenon. Magnetostriction (often called “Joule magnetostriction”) characterizes the expansion or contraction of a ferromagnetic material under magnetization. Positive magnetostrictive materials expand parallel to the direction of the magnetic field when magnetized, whereas negative magnetostrictive materials contract in the direction parallel to the magnetic field when magnetized.”

As is also disclosed in U.S. Pat. No. 6,363,793, “Materials which exhibit magnetostrictive properties will also exhibit the Villari effect. Materials with a positive magnetostriction coefficient suffer a decrease in magnetic permeability when subjected to compressive stresses, and will exhibit an increase in permeability when subjected to tensile stresses. The reverse occurs in negative magnetostrictive materials, i.e., permeability increases when compressive stresses are applied and decreases upon the application of tensile stress. This change in permeability or response magnetization of the material when stress is applied thereto is referred to as the Villari effect.”

As is also disclosed in U.S. Pat. No. 6,363,793, “Examples of positive magnetostrictive materials include iron, vanadium permendur (49% iron, 49% cobalt, 2% vanadium), or the permalloy (Nickel-iron) series of alloys. Terfenol-D is a ceramic material consisting of iron, terbium, and dysprosium specifically formulated to have an extremely high positive magnetostriction. Nickel is an example of a material with a negative magnetostriction coefficient. If a metallic alloy is used, the material must be properly annealed in order to remove work hardening effects and to ensure reasonable uniformity of the sensing material.

Referring again to FIG. 32, and to the preferred embodiment depicted therein, preferably disposed on the outer surface 5004 of the container 12, is a multiplicity of coatings, including a first coating of magnetostrictive material 5006 in which is disposed a first drug eluting polymer 5008, a second coating of magnetostrictve material 5010 in which is disposed a second drug eltuint polymer 5012, and a third coating of magnetostrictive material 5014 in which is disposed a third drug eluting polymer 5016.

Referring again to FIG. 32, disposed between coatings 5006 and 5008 is 5018 of nanomagnetic material; and disposed between 5008 from 5010 is nanomagnetic material 5019.

The coated device 5000 may be made, e.g., in substantial accordance with the procedure used to make semiconductor devices with different patterns of material on their surfaces. Thus, e.g., one can first mask the surface 5004, deposit the magnetostrictive material 5006, deposit the polymeric material on and in said magnetostrictive material, and thereafter, by changing the masking and the coatings, deposit the rest of the components.

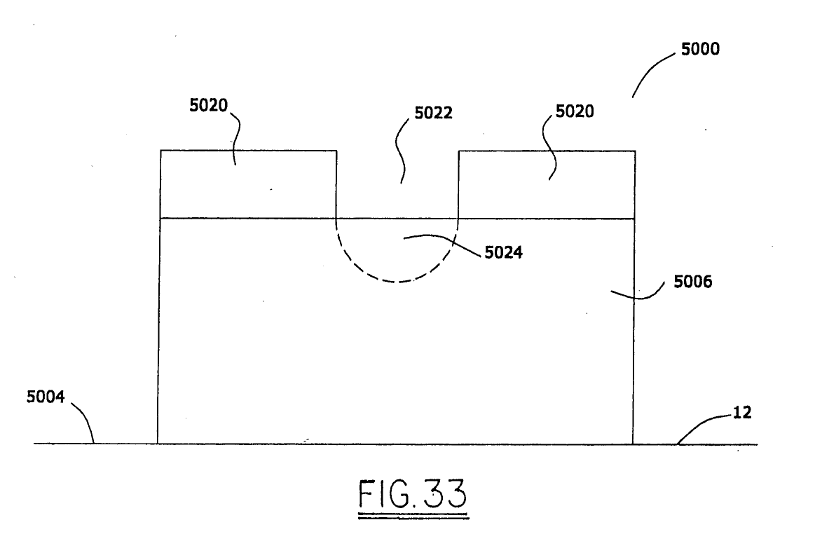

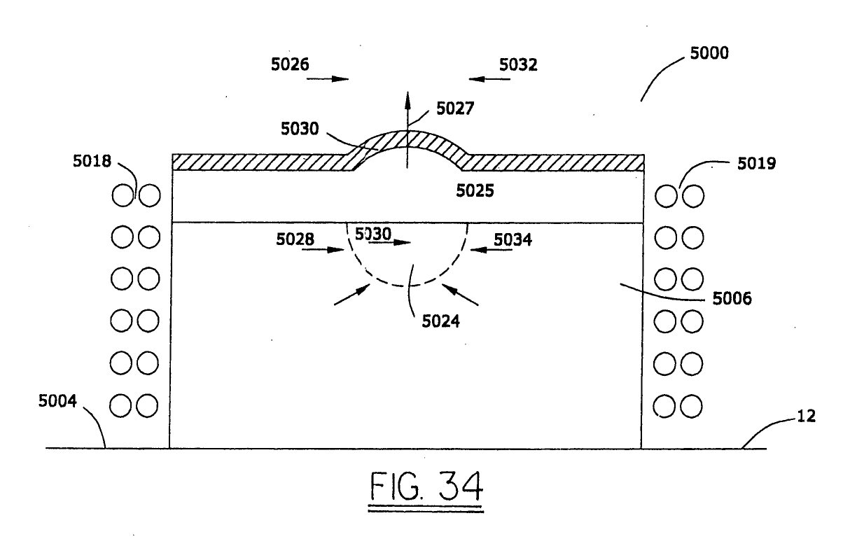

FIG. 33 is a partial view of magnetostrictive magnetostrictive material 5006 prior to the time an orifice has been created in it. In the embodiment depicted, a mask 5020 with an opening 5022 is disposed on top of the magnetostrictive material 5006, and an etchant (not shown) is disposed in said opening 5022 to create an orifice 5024, shown in dotted line outline. Thereafter, a drug-eluting polymer (such as, e.g., polymer 5008) is contacted with said etched surface and disposed within the orifice 5024. The resulting structure is shown in FIG. 34.

FIG. 34 shows the magnetostrictive material 50065 bounded by nanomagnetic material 5018/5019, and it illustrates how such assembly responds when the magnetostrictive material is subjected to one or more magnetic fields adapted to cause distortion of the material.

In the embodiment depicted in FIG. 34, a first direct current magnetic field 5026 causes force to act in the direction of arrow 5028, thereby causing distortion of the polymeric material 5024 in the direction of arrow 5030. When a second varying magnetic field 5032 (nominal direction) is applied, it causes force to act in the direction of arrow 5034. These fields, and others, may act simultaneously or sequentially to pump the material 5025 within orifice 5024 out of such orifice. The material 5025, in one embodiment, is cuased to move in the direction of arrow 5027, to cause a layer of material 5029 (which may be the same as or different than material 5025) to distend, and to thus rupture pressure rupturable seal 5030.

The pressure rupturable seal 5030 illustrated in FIG. 34 may be any of the pressure rupturable seals known to those skilled in the art. Reference may be had, e.g., to U.S. Pat. No. 3,787,075 (rupturable rubber seal), U.S. Pat. Nos. 3,810,655, 3,837,671 (“sealing means comprising a pressure-rupturable seal”), U.S. Pat. No. 4,220,259 (“pressure rupturable seal intermediate the contents and the boundary seal”), U.S. Pat. No. 4,622,033 (“ . . . lubricant reservoir is provided with seal means to prevent lubricant in the reservoir from drying during storage, said seal means being rupturable by pressure when lubricant is expressed from said reservoir by subjecting the lubricant to pressure . . . ”), U.S. Pat. No. 4,759,472 (“ . . . whereby upon application of predetermined external pressure to the container said weakly sealed area will rupture about said curvalinear side to permit the discharge of the packaged substance through said unsealed chamber and discharge spout, and a sealed diverter area within the unsealed chamber defined by said arcuate seal and discharge spout for retaining the walls of said container together at said diverter area upon rupturing of said weakly sealed area and for metering the discharge of the packaged substance through said unsealed chamber and discharge spout . . . ”), U.S. Pat. No. 4,785,972 (“ . . . a closed expandable vessel having a plurality of individual compartments formed by respective pressure-rupturable seal means therebetween, said compartments containing respective chemical compounds which when mixed upon the rupture of respective interfacing seal means produce a gas, and wherein at least two adjacent compartments respectively contain a first chemical compound aqueous solution and a second chemical compound aqueous solution which, when mixed upon the rupture of the seal means between said adjacent compartments, react with each other to produce a gas . . . ”, U.S. Pat. No. 4,808,346 (“ . . . a generally flat-sided flexible walled packet containing a predetermined individual serving quantity of a flavoring constituent and having a rupturable discharge end, said discharge end of said packet is formed with relatively strong permanently sealed areas which define an unsealed discharge spout, and an arcuate shaped sealed area surrounding said discharge spout for defining an unsealed chamber communicating with said discharge spout, whereby upon application of predetermined external pressure to the container said arcuate shaped sealed area will rupture to permit the controlled discharge of the packaged substance through said unsealed chamber and discharge spout . . . ”), U.S. Pat. No. 4,915,261 (“A sealed container for use in a beverage dispensing system having an actuating unit for applying a rupturing pressure to said container for dispensing a substance packaged therein, said container comprising walls of flexible material having mating peripheral edges, means forming a seal along a marginal area of said edges to define a fluid-tight internal packaging compartment, said marginal area seal including relatively strong permanently sealed areas which define an unsealed discharge spout, and an arcuate shaped sealed area surrounding said discharge spout for defining an unsealed chamber communicating with said discharge spout, whereby upon application of predetermined external pressure to the container by a beverage dispensing system actuating unit said arcuate sealed area will rupture to permit the controlled discharge of the packaged substance through said unsealed chamber and discharge spout.”), U.S. Pat. No. 4.919,310 (“ . . . A self-generating gas pressure apparatus for placement within a container from which a flowable material in the container is to be dispensed under pressure exerted on the material by the gas pressure apparatus and wherein said gas pressure apparatus comprises a closed expandable vessel having a plurality of individual compartments formed by respective pressure-rupturable seal means therebetween, said compartments containing respective chemical compounds which when mixed upon the rupture of respective interfacing seal means produce a gas, and wherein at least two adjacent compartments respectively contain a first water-soluble chemical compound in aqueous solution and a second precipitated chemical compound dispersed in a water-dispersible suspension medium . . . ”), U.S. Pat. No. 5,035,348 (“ . . . A fluid dispenser, the dispenser including a flexible vessel for containing a fluid, the vessel including i. a top wall and a bottom wall, and ii. means comprising a seal concentrating in a region thereof forces resulting from pressure generated in the fluid by applying a force to the vessel, said seal sealing the top wall to the bottom wall, said vessel being sufficiently strong that a weaker of the top wall or the bottom wall at the seal ruptures at the region of concentration in response to the applied force to form an opening through which the fluid is dispensed . . . ”), U.S. Pat. No. 5,158,546 (“ . . . means for axially driving the mixing container into the supplemental container in a controlled manner to force the second component past the seal into the variable volume mixing region causing the first and second components to mix and forcing the piston towards the first end to the post-mix position . . . ”),

An Implantable Medical Device with Minimal Susceptibility

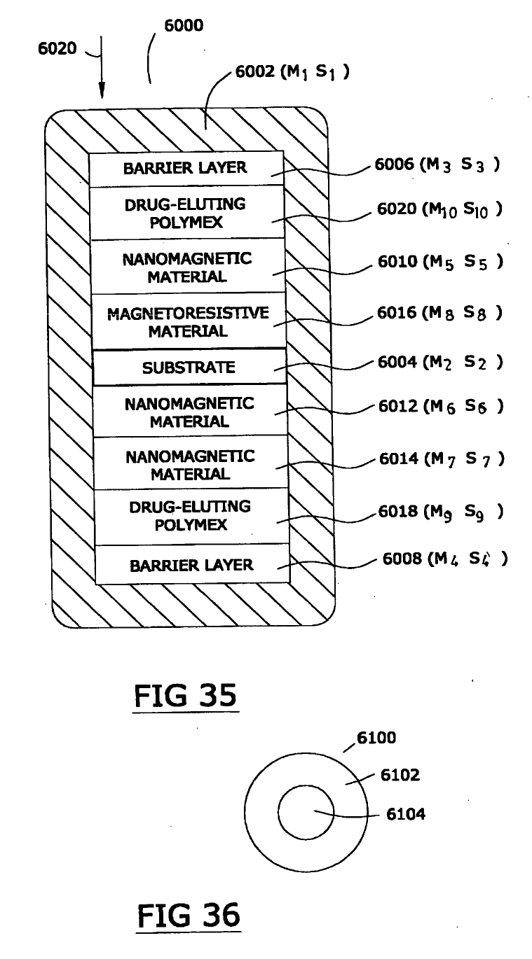

FIG. 35 presents a solution to a problem posed in published U.S. patent application 2004/0030379, the entire disclosure of which is hereby incorporated by reference into this specification. This published patent application discloses (at page 1 thereof) that: “In the medical field, magnetic resonance imaging (MRI) is used to non-invasively produce medical information. The patient is positioned in an aperture of a large annular magnet, and the magnet produces a strong and static magnetic field, which forces hydrogen and other chemical elements in the patient's body into alignment with the static field. A series of radio frequency (RF) pulses are applied orthogonally to the static magnetic field at the resonant frequency of one of the chemical elements, such as hydrogen in the water in the patient's body. The RF pulses force the spin of protons of chemical elements, such as hydrogen, from their magnetically aligned positions and cause the electrons to precess. This precession is sensed to produce electromagnetic signals that are used to create images of the patient's body. In order to create an image of a plane of patient cross-section, pulsed magnetic fields are superimposed on the high strength static magnetic field.”

Published U.S. patent application US 2004/0093075 also discloses that: “While researching heart problems, it was found that all the currently used metal stents distorted the magnetic resonance images of blood vessels. As a result, it was impossible to study the blood flow in the stents and the area directly around the stents for determining tissue response to different stents in the heart region.

Published U.S. patent application 2004/0093075 also discloses that: “A solution, which would allow the development of a heart valve which could be inserted with the patients only slightly sedated, locally anesthetized, and released from the hospital quickly (within a day) after a procedure and would allow the in situ magnetic resonance imaging of stents, has long been sought but yet equally as long eluded those skilled in the art.” Such a solution is disclosed in FIG. 35 of the instant application.

The device 6000 depicted in FIG. 35, in one embodiment, is an assembly comprised of a device and material within which such device is disposed, wherein the direct current magnetic susceptibility of such assembly is plus or minus 1×10⁻³.

Referring to FIG. 35, there is disclosed an assembly 6000 comprised of a first material 6002 (with a first mass [M₁] and a first magnetic susceptibility [S₁]) that, in the embodiment depicted, is contiguous with a substrate 6004 (with a second mass [M₂] and a second magnetic susceptibility [S2]).

In one preferred embodiment, the substrate 6004 is an implantable medical device. Thus, and as is disclosed in published U.S. patent application 2004/0030379 (the entire disclosure of which is hereby incorporated by reference into this specification), the implanted medical device may be a stent. Thus, and referring to page 4 of such published patent application, “Medical devices which are particularly suitable for the present invention include any kind of stent for medical purposes, which are known to the skilled artisan. Suitable stents include, for example, vascular stents such as self-expanding stents and balloon expandable stents. Examples of self-expanding stents useful in the present invention are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 4,733,665 issued to Palmaz, U.S. Pat. No. 4,800,882 issued to Gianturco, U.S. Pat. No. 4,886,062 issued to Wiktor and U.S. Pat. No. 5,449,373 issued to Pinchasik et al. A bifurcated stent is also included among the medical devices suitable for the present invention.”

As is also disclosed in published U.S. patent application 2004/0030379. “The medical devices suitable for the present invention may be fabricated from polymeric and/or metallic materials. Examples of such polymeric materials include polyurethane and its copolymers, silicone and its copolymers, ethylene vinyl-acetate, poly(ethylene terephthalate), thermoplastic elastomer, polyvinyl chloride, polyolephines, cellulosics, polyamides, polyesters, polysulfones, polytetrafluoroethylenes, acrylonitrile butadiene styrene copolymers, acrylics, polyactic acid, polyclycolic acid, polycaprolactone, polyacetal, poly(lactic acid), polylactic acid-polyethylene oxide copolymers, polycarbonate cellulose, collagen and chitins. Examples of suitable metallic materials include metals and alloys based on titanium (e.g., nitinol, nickel titanium alloys, thermo-memory alloy materials), stainless steel, platinum, tantalum, nickel-chrome, certain cobalt alloys including cobalt-chromium-nickel alloys (e.g., Elgiloy® and Phynox®) and gold/platinum alloy. Metallic materials also include clad composite filaments, such as those disclosed in WO 94/16646.”

In one preferred embodiment, the substrate 6004 is a conventional drug-eluting medical device (such as, e.g., a drug eluting stent) to which the nanomagnetic material of this invention has been added as described hereinbelow. One may use, and modify, any of the prior art self-eluting medical devices.

By way of illustration, and as is disclosed in U.S. Pat. Nos. 5,591,227, 5,599,352, and 6,597,967 (the entire disclosure of each of which is hereby incorporated by reference into this specification), the medical device may be “ . . . a drug eluting intravascular stent comprising: (a) a generally cylindrical stent body; (b) a solid composite of a polymer and a therapeutic substance in an adherent layer on the stent body; and (c) fibrin in an adherent layer on the composite.” In the device of U.S. Pat. No. 5,591,227, the fibrin was used to provide a biocompatible surface. In the device 6000 depicted in FIG. 35, it may be used as, or in place of barrier layer 6006 and/or barrier layer 6008.

By way of yet further illustration, and and as is disclosed in U.S. Pat. No. 6,623,521 (the entire disclosure of which is hereby incorporated by reference into this specification), the medical device may be an expandable staent with sliding and locking radial elements. This patent discloses many “prior art” stents, whose designs also may be modified by the inclusion of nanomagnetic material. Thus as is disclosed at columns 1-2 of this patent, “Examples of prior developed stents have been described by Balcon et al., “Recommendations on Stent Manufacture, Implantation and Utilization,” European Heart Journal (1997), vol. 18, pages 1536-1547, and Phillips, et al., “The Stenter's Notebook,” Physician's Press (1998), Birmingham, Mich. The first stent used clinically was the self-expanding “Wallstent” which comprised a metallic mesh in the form of a Chinese fingercuff. This design concept serves as the basis for many stents used today. These stents were cut from elongated tubes of wire braid and, accordingly, had the disadvantage that metal prongs from the cutting process remained at the longitudinal ends thereof. A second disadvantage is the inherent rigidity of the cobalt based alloy with a platinum core used to form the stent, which together with the terminal prongs, makes navigation of the blood vessels to the locus of the lesion difficult as well as risky from the standpoint of injury to healthy tissue along the passage to the target vessel. Another disadvantage is that the continuous stresses from blood flow and cardiac muscle activity create significant risks of thrombosis and damage to the vessel walls adjacent to the lesion, leading to restenosis. A major disadvantage of these types of stents is that their radial expansion is associated with significant shortening in their length, resulting in unpredictable longitudinal coverage when fully deployed.”

As is also disclosed in U.S. Pat. No. 6,623,521 “Among subsequent designs, some of the most popular have been the Palmaz-Schatz slotted tube stents. Originally, the Palmaz-Schatz stents consisted of slotted stainless steel tubes comprising separate segments connected with articulations. Later designs incorporated spiral articulation for improved flexibility. These stents are delivered to the affected area by means of a balloon catheter, and are then expanded to the proper size. The disadvantage of the Palmaz-Schatz designs and similar variations is that they exhibit moderate longitudinal shortening upon expansion, with some decrease in diameter, or recoil, after deployment. Furthermore, the expanded metal mesh is associated with relatively jagged terminal prongs, which increase the risk of thrombosis and/or restenosis. This design is considered current state of the art, even though their thickness is 0.004 to 0.006 inches.”

As is also disclosed in U.S. Pat. No. 6,623,521, “Another type of stent involves a tube formed of a single strand of tantalum wire, wound in a sinusoidal helix; these are known as coil stents. They exhibit increased flexibility compared to the Palnaz-Schatz stents. However, they have the disadvantage of not providing sufficient scaffolding support for many applications, including calcified or bulky vascular lesions. Further, the coil stents also exhibit recoil after radial expansion.”

As is also disclosed in U.S. Pat. No. 6,623,521, “One stent design described by Fordenbacher, employs a plurality of elongated parallel stent components, each having a longitudinal backbone with a plurality of opposing circumferential elements or fingers. The circumferential elements from one stent component weave into paired slots in the longitudinal backbone of an adjacent stent component. By incorporating locking means within the slotted articulation, the Fordenbacher stent may minimize recoil after radial expansion. In addition, sufficient numbers of circumferential elements in the Fordenbacher stent may provide adequate scaffolding. Unfortunately, the free ends of the circumferential elements, protruding through the paired slots, may pose significant risks of thrombosis and/or restenosis. Moreover, this stent design would tend to be rather inflexible as a result of the plurality of longitudinal backbones.”

As is also disclosed in U.S. Pat. No. 6,623,521, “Some stents employ “jelly roll” designs, wherein a sheet is rolled upon itself with a high degree of overlap in the collapsed state and a decreasing overlap as the stent unrolls to an expanded state. Examples of such designs are described in U.S. Pat. No. 5,421,955 to Lau, U.S. Pat. Nos. 5,441,515 and 5,618,299 to Khosravi, and U.S. Pat. No. 5,443,500 to Sigwart. The disadvantage of these designs is that they tend to exhibit very poor longitudinal flexibility. In a modified design that exhibits improved longitudinal flexibility, multiple short rolls are coupled longitudinally. See e.g., U.S. Pat. No. 5,649,977 to Campbell and U.S. Pat. Nos. 5,643,314 and 5,735,872 to Carpenter. However, these coupled rolls lack vessel support between adjacent rolls.”

As is also disclosed in U.S. Pat. No. 6,623,521, “Another form of metal stent is a heat expandable device using Nitinol or a tin-coated, heat expandable coil. This type of stent is delivered to the affected area on a catheter capable of receiving heated fluids. Once properly situated, heated saline is passed through the portion of the catheter on which the stent is located, causing the stent to expand. The disadvantages associated with this stent design are numerous. Difficulties that have been encountered with this device include difficulty in obtaining reliable expansion, and difficulties in maintaining the stent in its expanded state.”

As is also disclosed in U.S. Pat. No. 6,623,521, “Self-expanding stents are also available. These are delivered while restrained within a sleeve (or other restraining mechanism), that when removed allows the stent to expand. Self-expanding stents are problematic in that exact sizing, within 0.1 to 0.2 mm expanded diameter, is necessary to adequately reduce restenosis. However, self-expanding stents are currently available only in 0.5 mm increments. Thus, greater selection and adaptability in expanded size is needed.”

The stent design claimed in U.S. Pat. No. 6,623,521 is: An expandable intraluminal stent, comprising: a tubular member comprising a clear through-lumen, and having proximal and distal ends and a longitudinal length defined there between, a circumference, and a diameter which is adjustable between at least a first collapsed diameter and at least a second expanded diameter, said tubular member comprising: at least one module comprising a series of radial elements, wherein each radial element defines a portion of the circumference of the tubular member and wherein no radial element overlaps with itself in either the first collapsed diameter or the second expanded diameter; at least one articulating mechanism which permits one-way sliding of the radial elements from the first collapsed diameter to the second expanded diameter, but inhibits radial recoil from the second expanded diameter; and a frame element which surrounds at least one radial element in each module.”

By way of yet further illustration, one may use the multi-coated drug-eluting stent described in U.S. Pat. No. 6,702,850, the entire disclosure of which is hereby incorporated by reference in to this specification. This patent describes and claims: “ . . . a stent body comprising a surface; and a coating comprising at least two layers disposed over at least a portion of the stent body, wherein the at least two layers comprise a first layer disposed over the surface of the stent body and a second layer disposed over the first layer, said first layer comprising a polymer film having a biologically active agent dispersed therein, and the second layer comprising an antithrombogenic heparinized polymer comprising a macromolecule, a hydrophobic material, and heparin bound together by covalent bonds, wherein the hydrophobic material has more than one reactive functional group and under 100 mg/ml water solubility after being combined with the macromolecule.”

Referring again to FIG. 35, and to the preferred embodiment depicted therein, the substrate 6004 (such as, e.g., an implantable stent) is disposed within material 6002. The material is preferably biological material, such as the biological material disclosed in published U.S. patent application 2004/0030379. Thus, and as is disclosed in such published patent application, “The present invention provides a method of treatment to reduce or prevent the degree of restenosis or hyperplasia after vascular intervention such as angioplasty, stenting, atherectomy and grafting. All forms of vascular intervention are contemplated by the invention, including, those for treating diseases of the cardiovascular and renal system. Such vascular intervention include, renal angioplasty, percutaneous coronary intervention (PCI), percutaneous transluminal coronary angioplasty (PTCA); carotid percutaneous transluminal angioplasty (PTA); coronary by-pass grafting, angioplasty with stent implantation, peripheral percutaneous transluminal intervention of the iliac, femoral or popliteal arteries, carotid and cranial vessels, surgical intervention using impregnated artificial grafts and the like. Furthermore, the system described in the present invention can be used for treating vessel walls, portal and hepatic veins, esophagus, intestine, ureters, urethra, intracerebrally, lumen, conduits, channels, canals, vessels, cavities, bile ducts, or any other duct or passageway in the human body, either in-born, built in or artificially made. It is understood that the present invention has application for both human and veterinary use.”

Thus, in one embodiment, the material 6002 is biological material such as, e.g., blood, fat cells, muscle, etc.

Referring again to FIG. 35, and to the preferred embodiment depicted therein, a layer of magnetoresistive material 6016 is disposed over the substrate 6004. As is known to those skilled in the art, magnetoresitance is the change in electrical resistance produced in a current-carrying conductor or semi-conductor upon the application of a magnetic field. Reference may be had, e.g., to U.S. Pat. Nos. 6,064,552; 6,178,072; 6,219,205; 6,243,288; 6,256,177; 6,292,336; 6,329,818; 6,340,520 (giant magnetorestive film); U.S. Pat. Nos. 6,387,550; 6,396,734 6,433,792; 6,452,382; 6,483,740; 6,490,140; 6,498,707; 6,501,271 (magnetoresitive effect multilayer sensor); U.S. Pat. Nos. 6,519,119; 6,538,430; 5,538,859; 6,574,061; 6,589,366 (giant magnetoresisstance materials based upon Gd—Si—Ge alloys), U.S. Pat. Nos. 6,594,175; 6,612,018; 6,621,667 (giant magnetoresistrive sensor), U.S. Pat. Nos. 6,674,664; -6,717,778; 6,730,036 (giant magnetoresistive thin film); and the like. The entire disclosure of each of these U.S. patents is hereby incorporated by reference into this specification.

Without wishing to be bound to any particular theory, applicants believe that the presence of the magnetoresistive material 6004 helps minimize the presence of eddy currents in substrate 6004 when the assembly 6000 is subjected to a magnetic resonance imaging (MRI) field 6020.

In one preferred embodiment, illustrated in FIG. 35, layers of barrier material 6006 and 6008 are disposed over drug eluting polmer materials 6020 and 6018, respectively. This barrier material is described in U.S. Pat. No. 6,716,444, the entire disclosure of which is hereby incorporated by reference into this specification.

Claim 16 of U.S. Pat. No. 6,716,444 discloses: “16. An implantable medical device comprising: a substrate; a polymer coating containing a drug said polymer disposed on said substrate; and a barrier overlaying at least a portion of said coating, wherein said barrier comprises an inorganic material and is adapted to reduce a rate of release of said drug from said coating after insertion of said device into a body of a patient, wherein said inorganic material is selected from the group consisting of carbides of silicon, carbides of titanium, molybdenum disulfide, amorphous diamond, diamondlike carbon, pyrolytic carbon, ultra low temperature isotropic carbon, amorphous carbon, strontium titanate, and barium titanate.”

As is disclosed in column 3 of U.S. Pat. No. 6,716,444, “The present invention allows for a controlled rate of release of a drug or drugs from a polymer carried on an implantable medical device. The controlled rate of release allows localized drug delivery for extended periods, e.g., weeks to months, depending upon the application. This is especially useful in providing therapy to reduce or prevent cell proliferation, inflammation, or thrombosis in a localized area.”

As is also disclosed in U.S. Pat. No. 6,716,444, “One embodiment of an implantable medical device in accordance with the present invention includes a substrate, which may be, for example, a metal or polymeric stent or graft, among other possibilities. At least a portion of the substrate is coated with a first layer that includes one or more drugs in a polymer carrier. A barrier coating overlies the first layer. The barrier (which may be considered a coating) reduces the rate of release of the drug from the polymer once the medical device has been placed into the patient's body, thereby allowing an extended period of localized drug delivery once the medical device is in situ.”

As is also disclosed in U.S. Pat. No. 6,714,444, “The barrier is necessarily biocompatible (i.e., its presence does not elicit an adverse response from the body), and typically has a thickness ranging from about 50 angstroms to about 20,000 angstroms. It is contemplated that the barrier contains mostly inorganic material. However, some organic compounds (e.g., polyacrylonitrile, polyvinylidene chloride, nylon 6-6, perfluoropolymers, polyethylene terephthalate, polyethylene 2,6-napthalene dicarboxylate, and polycarbonate) may be incorporated in the barrier. Suitable inorganic materials for use within the barrier include, but are not limited to, inorganic elements, such as pure metals including aluminum, chromium, gold, hafnium, iridium, niobium, palladium, platinum, tantalum, titanium, tungsten, zirconium, and alloys of these metals, and inorganic compounds, such as inorganic silicides, oxides, nitrides, and carbides. Generally, the solubility of the drug in the material of the barrier is significantly less than the solubility of the drug in the polymer carrier. Also, generally, the diffusivity of the drug in the material of the barrier is significantly lower than the diffusivity of the drug in the polymer carrier.”

In one preferred embodiment, the diffusivity of the drug through the barrier layer is affected by the application of an external electromagnetic field. The external magnetic field (such as, e.g., field 6020) may be used to heat the nanomagnetic material 6010 and/or the nanomagnetic material 6012 and/or the magnetoresitive material 6016, which in turn will tend to heat the drug eluting polymer 6018 and/or the drug eluting polymer 6020 and/or the barrier layer 6008 and/or the barrier layer 6006. To the extent that such heating increases the diffusion of the drug from the drug-eluting polymer, one may increase the release of such drug from such drug-eluting polymer.

In one embodiment, illustrated in FIG. 35, The heating of the nanomagnetic material 6010 and/or 6012 decreases the effectiveness of the barrier layers 6006 and/or 6008 and, thereby, increases the rate of drug delivery from drug-eluting polymers 6020 and/or 6018.

Referring again to FIG. 35, when an MRI MRI field 6020 is present, the entire assembly 6000, including the biological material 6020, presents a direct current magnetic susceptibility that preferably is plus or minus 1××10⁻³ centimeter-gram-seconds (cgs) and, more preferably, plus or minus 1×10⁻⁴ centimeter-gram-seconds. In one embodiment, the d.c. susceptibility of the stent is equal to plus or minus 1×10⁻⁵ centimeter-gram-seconds. In another embodiment, the d.c. susceptibility of the stent is equal to plus or minus 1×10⁻⁶ centimeter-gram-seconds.

Referring again to FIG. 35, each of the components of assembly 6000 has its own value of magnetic susceptibility. The biological material 6002 has a magnetic susceptibility of S₁. The substrate 6012 has a magnetic susceptibility of S₂. The magnetoresistive 6016 material has a magnetic susceptibility of S₃. The drug-eluting polymeric materials 6018 and 6020 have magnetic susceptibilies of S₉ and S₁₀, respectively.

Each of the components of the assembly 6000 makes a contribution to the total magnetic susceptibility of such assembly, depending upon (a) whether its magnetic susceptibility is positive or negative, (b) the amount of its positive or negative susceptibility value, and (c) the percentage of the total mass that the individual coponenent represents.

In determining the total susceptibility of the assembly 6000, one can first determine the product of Mc and Sc, wherein Mc is the weight fraction of that component (the weight of that component divided by the total weight of all components in the assembly 6000).

In one preferred process, the McSc values for the nanomagentic material 6016 and the nanomagnetic material 6012 are chosen to, when appropriate, correct for the total McSc values of all of the other components (including the biological material 6002 such that, after such correction(s), the total susceptibility of the assembly 6000 is plus or minus 1××10⁻³centimeter-gram-seconds (cgs) and, more preferably, plus or minus 1×10⁻⁴ centimeter-gram-seconds. In one embodiment, the d.c. susceptibility of the assembly 6000 is equal to plus or minus 1×10⁻⁵ centimeter-gram-seconds. In another embodiment, the d.c. susceptibility of the assembly 6000 is equal to plus or minus 1×10⁻⁶ centimeter-gram-seconds.

As will be apparent, there may be other materials/components in the assembly 6000 whose values of positive or negative susceptibility, and/or their mass, may be chosen such that the total magnetic susceptibility of the assembly is plus or minus 1××10⁻³ centimeter-gram-seconds (cgs) and, more preferably, plus or minus 1×10⁻⁴ centimeter-gram-seconds. Similarly, the configuration of the substrate may be varied in order to vary its magnetic susceptibility properties and/or other properties. One of these variations is depicted in FIG. 36.

As is known to those skilled in the art, many stents comprise wire. See, e.g., U.S. Pat. No. 6,723,118 (flexible metal wire stent), U.S. Pat. No. 6,719,782 (flat wire stent), U.S. Pat. No. 6,525,574 (wire stent coated with a biocompatible fluoropolymer), U.S. Pat. Nos. 6,579,308, 6,375,660, 6,161,399 (wire reinforced monolayer fabric stent), U.S. Pat. No. 6,071,308 (flexible metal wire stent), U.S. Pat. No. 6,056,187 (modular wire band stent), 5,999,482 (flat wire stent), U.S. Pat. No. 5,906,639 (high strength and high density intralumina wire stent), and the like. The entire disclosure of each of these U.S. patents is hereby incorporated by reference into this specification.

FIG. 36 is a sectional view of a wire 6100 which may be used to replace the wire used in conventional metal wire stents. The wire 6100 preferably has a sheath/core arrangement, with sheath 6102 disposed about core 6104.

In one embodiment, the materials chosen for the sheath 6102 and/or the core 6104 afford one both the desired mechanical properties as well as a magnetic susceptibility that, in combination with the other components of the assembly (and of the biological tissue), produce a magnetic susceptibility of plus or minus 1×10⁻³ cgs.

In another embodiment, the matrials chosen for the sheath 6102 and/or the core 6104 are preferably magnetoresistive and produce a high resistance when subjected to MRI radiation.

Another Drug Eluting Device

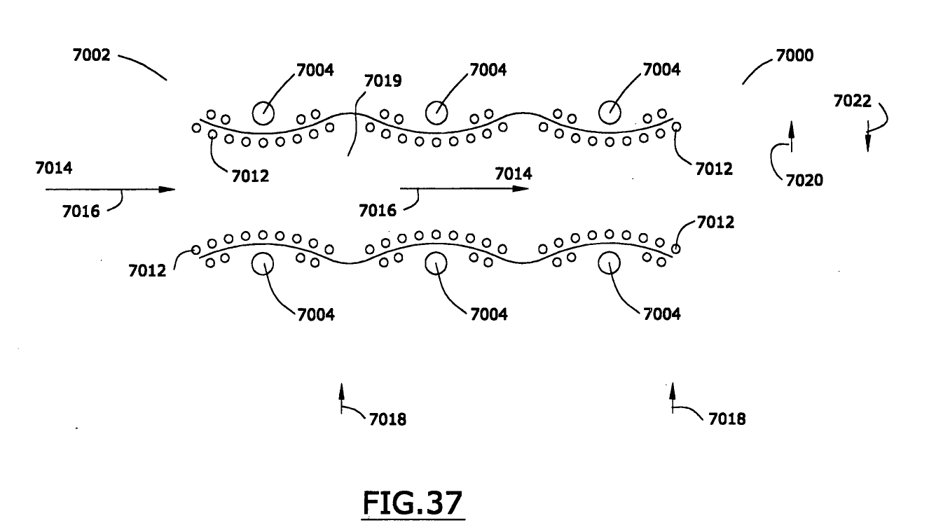

FIG. 37 is sectional view of a device 7000 comprised of a stent 7002. Although a stent is illustrated as the device in FIG. 37, it will be apparent that other devices, especially other implantable devices, also may be used.

The device of FIG. 37 may be used with any conventional drug eluting stent. platform. Thus, by way of illustration, one may use the “NIRx” stent platform described at pages 312 of Patrick W. Serruys et al.'s “Handbook of Coronary Stents,” Fourth Edition (Martin Dunitz Ltd., London, United Kingdom, 1998). As is disclosed in such text, this platform “ . . . is a premounted monorail (single operator exchange) system and combines the mechanical advantages of the NIR . . . stent with a drug coating system . . . . Currently the NIR . . . is available for clinical evaluation premounted on Boston Scientific's Advance catheter system . . . . The distal section of the catheter has a dual lumen; the outer lumen is used for balloon inflation and the inner accepts a 0.014 inch guidewire. The Advance system is a monorail style stent delivery system . . . . The proximal section is a single stainless steel hypotube with a single luer port for inflation. The tip is tapered to facilate advancement. The catheter will deliver 15 mm length stents with 3.0 mm and 3.5 mm diameter options . . . .”

Referring again to FIG. 37, and in the preferred embodiment depicted therein, it will be seen that stent 7002 is compmrised of a multiplicity of stent struts 7004. The struts of stents are well known to those skilled in the art, as is disclosed, e.g., in U.S. Pat. No. 5,843,168 (double wave stent with strut); U.S. Pat. No. 5,911,754 (flexible stent with effective strut and connector patterns); U.S. Pat. No. 5,913,895 (intravascular stent with enhanced rigidity strut members); U.S. Pat. No. 6,113,627 (tubular stent consisting of horizontal expansion struts); U.S. Pat Nos. 6,123,721; 6,129,755 (intravascular stent having an improved strut configuration); U.S. Pat. No. 6,190,406 (intravascular stent having tapered struts); U.S. Pat. No. 6,235,053 (tubular stent consisting of chevron-shaped expansion struts and contralaterally attached diagonal connectors); U.S. Pat. No. 6,273,910 (stent with varying strut geometry); U.S. Pat. No. 6,273,913 (modified stent useful for delivery of drugs along stent strut); U.S. Pat. No. 6,475,233 (stent having tapered struts); U.S. Pat. No. 6,579,310 (stent having overlapping stuts); and the like. The entire disclosure of each of these U.S. patents is hereby incorporated by reference into this specification.

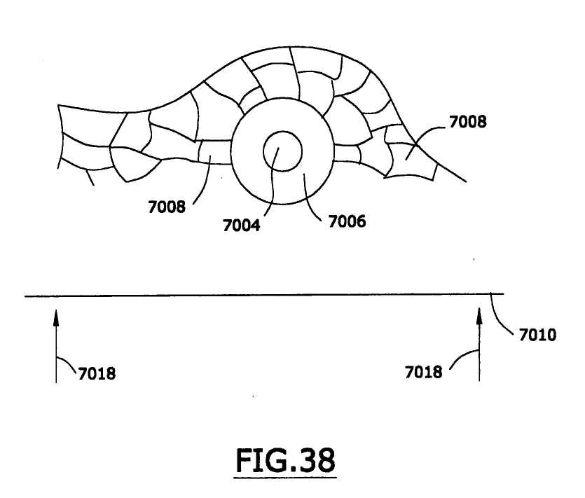

FIG. 38 is an enlarged sectional view of one of the struts 7004 and its preferred associated components. Referring to FIG. 38, and in the embodiment depicted, the strut 7004 is coated with a coating 7006 of nanomagnetic material; such nanomagnetic material preferably has the properties described elsewhere in this specification.

The strut 7004 preferably is contiguous with, or disposed near, endothelial cells 7008. As is known to those skilled in the art, endothelial cells are cells that form the lining of blood vessels.

In the embodiment depicted in FIG. 38 (and also in FIG. 37), the stent 7002 and its struts 7004 has been endothelialized, i.e., a layer of endothelial cells 7008 has grown over the struts 7004 and its associated coating 7006.Although omitted for the sake of simplicity of representation, these endothelial cells 7008 are often contiguous with and comprise the blood vessel inner wall 7010. As will be apparent, FIG. 38 is not drawn to scale and is merely a partial schematic representation of endothelial cells 7008 surrounding the strut 7004.

Referring again to FIG. 37, and to the preferred embodiment depicted therein, a multiplicity of magnetic drug particles 7012 is disposed near each of the struts 7004. In the embodiment depicted in FIG. 37, for ease of simplicity of representation, the coating 7006 is not shown being disposed about the struts 7004.

In one preferred embodiment, the magnetic particles 7012 preferably have a saturation magnetization (“magnetic moment”) of from about 2 to about 3,000 electromagnetic units (emu) per cubic centimeter of material. Although it is preferred that the particles 7012 be the nanomagnetic particles described elsewhere in this speificaiton, they may be other magnetic particles as long as they possess the desired degree of magnetization.

As is known to those skilled in the art, magnetization is the magnetic moment per unit volume of a substance. Reference may be had, e.g., to U.S. Pat. Nos. 4,169,998, 4,168,481, 4,166,263, 5,260,132, 4,778,714, and the like. The entire disclosure of each of these U.S. patents is hereby incorporated by reference into this specification.

In one preferred embodiment, the saturation magnetization of the magnetic particles 7012 is at least 100 electromagnetic units (emu) per cubic centimeter and, more preferably, at least about 200 electromagnetic units (emu) per cubic centimter. In one aspect of this embodiment, the saturation magnetization of such nanomagnetic particles is at least about 1,000 electromagnetic units per cubic centimeter.

In another embodiment, the saturation magnetization of the magnetic particles 7012 is at least about 2,000 electromagnetic units per cubic centimeter and, more preferably, at least about 2,500 electromagnetic units per cubic centimeter. In this embodiment, the magnetic material in the particles is preferably nanomagnetic material, and it has the formula A₁A₂Bₓ (C₃ C₄) the C moieties are oxygen and nitrogen, respectively. This material is described elsewhere in this specification.

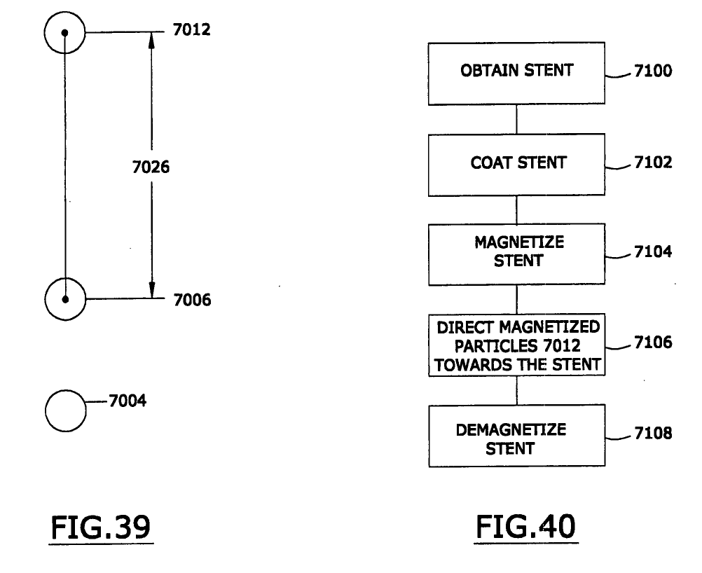

Referring again to FIG. 37, it will be seen that blood 7014 flows through the stent 7002 in the direction of arrow 7106. As such blood flows, the hydrodynamic forces of such flow tend to cause one or more of the particles 7012 to become dislodged from the stent, under the influence of the both the hydrodynamic forces involved, the magnetic forces between the coating 7006 (see FIG. 38) and the particles 7012, and/or the forces between the particles 7012 and an externally applied electromagnetic field 7018. As will be apparent, the action of any particular particle 7012 will be due to a force balance (or imbalance) of these factors. When the force imbalance is in the directon of arrow 7020, and referring to the “upper half” 7019 of such stent, the particles will tend to bind to the stent 7002 and its struts 7004. When the force imbalance is in the direction of arrow 7022, and again referring to such “upper half” 7019 of such stent 7002, the particles will tend to elute from and/or be repelled by the stent 7002 and/or its struts 7004. FIG. 39 schematically illustrates the forces between a portion of the nanomagentic coating 7006 preferably disposed about each struct 7004 and the magnetic particles 7012. The nanomagnetic coating 7006 preferably has the properties described elsewhere in this specification. Thus, e.g., in one embodiment, the nanomagnetic coating 7006 preferably is comprised of nanomagentic particles have an average particle size of less than about 100 nanometers. In one aspect of this embodiment, the average coherence length between adjacent nanomagnetic particles is less than 100 nanometers.

Thus, e.g., in another embodiment, the nanomagnetic coating 7006 is comprised of nanomagnetic material with a saturation magentization of from about 2 to about 3000 electromagnetic units per cubic centimeter and a phase transition temperature of from about 40 to about 200 degrees Celsius. In one aspect of this embodiment, the saturization magnetization of coating 7006 is at least 100 electromagnetic units (emu) per cubic centimeter and, more preferably, at least about 200 electromagnetic units (emu) per cubic centimter. In another aspect of this embodiment, the saturation magnetization of such nanomagnetic coating 7006 is at least about 1,000 electromagnetic units per cubic centimeter. In yet another aspect of this embodiment, the saturation magnetization of the nanomagnetic coating 7006 is at least about 2,000 electromagnetic units per cubic centimeter and, more preferably, at least about 2,500 electromagnetic units per cubic centimeter.

Referring to FIG. 39, and in the preferred embodiment depicted, the coating 7006 and the particles 7012 have magnetic moments (not shown) M1 and M2 respectively. The magnetic moment of the coating 7006 is preferably greater than the magnetic moment of the particles 7012, preferably being at least one order of magnitude larger. Thus, the particles 7012, with their lesser magnetic moments, tend to become magnetically aligned with the coating 7006.

Referring again to FIG. 39, the distance 7026 between the particles 7012 and the coating 7006 is preferably less than about 1 centimeter, and more preferably less than about 0.5 centimeters. As is known, and in accordance with the inverse square law, the force between the coating 7006 and the particles 7012 is dependent upon the product of the magnetic moments of the coating and the particles divided by the square of the distance 7026.

In order to obtain the status depicted in FIGS. 37, 38, and 39, certain steps must first be taken. These steps are illustrated in the flow diagram presented as FIG. 40.

Referring to Figure, and in step 7100 thereof, a stent is obtained. The stent may be a “coronary stent.” Many such “coronary stents” are disclosed in Patrick W. Serruys et al.'s “Handbook of Coronary Stents,” Fourth Edition (Martin Dunitz Ltd., London, England, 2002).

Thereafter, in step 7102, the stent is coated with one or of a coatings of nanomagnetic material, and/or nanomagnetostrictive material, and/or nanomagnetoresistive material, and/or organic polymeric material. During one or more of these coating processes, or thereafter, the stent is magnetized in step 7104.

The stent preferably is comprised of magetizable material, such as the nanomagentic coating 7006 (see FIG. 38). The magnetization process may be conducted by conventional means such as, e.g., the means dislosed in U.S. Pat. No. 4,409,581(process and apparatus for magnetizing, on both sides, the surfaces of bodies to be magnetized); U.S. Pat. No. 4,743,849 (magnetizing device for recroding flaw fields in the process of magnetograpic inspection); U.S. Pat. No. 5,466,180 (process and deivce for magnetizing a magnet ring in the neck of a color picture tube); U.S. Pat. No. 5,828,189 (process and apparatus for magnetizing a magnet ring); U.S. Pat. No. 6,380,654 (process for magnetizing the permanent magnets of an electric motor rotor); and the like. The entire disclosure of each of these United States patents is hereby incorporated by reference into this specification.

Referring again to FIG. 40, after the stent (or other medical device) has been magnetized in step 7104, in step 7106 a multiplicity of magnetic particles 7012 are directed towards the magnetized stent.

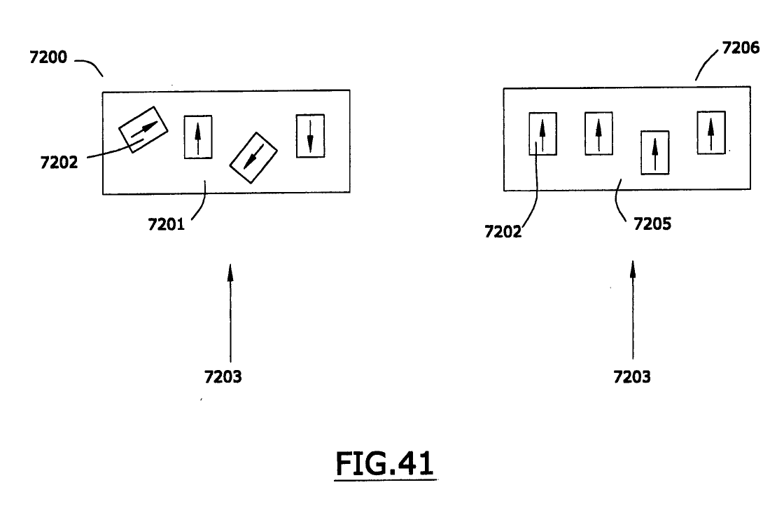

FIG. 41 schematically illustrates what occurs during the magnetization process. Referring to FIG. 41, a randomly aligned object 7200 with randomly disposed magnetic clusters 7202 is shown in unmagnetized state 7201. After an externally applied direct current field 7203 is contacted with clusters 7202, they align in accordance with the manner shown in object 7206. The external field 7203 preferably has a magnetic field strength of from between about 0.5 Gauss to about 40 Tesla. In one embodiment, the strength of the field 7203 is from about 50 to about 150 Gauss.

In one preferred embodiment, the nanomagnetic coating 7006 (see FIG. 38) preferably has a squareness of from about 0.001 to about 1 and, more preferably, from about 0.1 to about 0.5. As is known to those skilled in the art, the squareness of a magnetic material is the ratio of the residual magnetic flux and the saturation magnetic flux density; see, e.g., the discussion of squareness presented elsewhere in this specification.

As is known to those skilled in the art, the higher the squareness of a magnetic material, the longer it retains its magnetization. It is thus preferred, in one embodiment, that the nanomagnetic material in coating 7006 have a squareness of at least 0.5 and, more preferably, at least about 0.8. In applications where a large residual magnetic moment is desired, the squareness is preferably at least about 0.9.

Referring again to FIG. 40, and in optional step 7108, one may optionally demagnetize the stent by conventional means. Thus, e.g., one may use one or more of the demagnetizing processes described in U.S. Pat. No. 4,384,313, the entire disclosure of which is hereby incorporated by reference into this specification. This patent claims “1. In a process for the demagnetization of components subjected to the influence of an alternating magnetic field of a coil connected with capacitor means in an oscillator circuit supplied by an alternating voltage means, comprising the steps of: (a) placing a component to be demagnetized in the vicinity of said coil to be subjected to an alternating magnetic field generated by said coil; (b) varying the phase between voltage and current of the oscillator circuit to produce a control voltage to bring the frequency of the supplied voltage from a non-resonant frequency to the resonant frequency of the oscillator circuit, and; (c) thereafter reducing the intensity of the alternating magnetic field to which said component is subjected.” This patent discloses, in its “background of the invention” section, that: “The invention concerns a process for the demagnetization or for the magnetic calibration of parts of ferromagnetic materials, in particular for the demagnetization or calibration of permanent magnets, as well as for the demagnetization of components that have been exposed to a magnetic field during processing and have retained a residual magnetism from it, for example parts that have been ground on magnetic clamping plates, or chucks, or parts that are to be totally free of residual magnetism, such as ball bearings. A known demagnetizing process consists of exposing such parts to an alternating magnetic field of decreasing intensity, for example to conduct them through the field of an AC-powered coil or to expose them within a coil to the decreasing alternating field of a periodic capacitor discharge.”

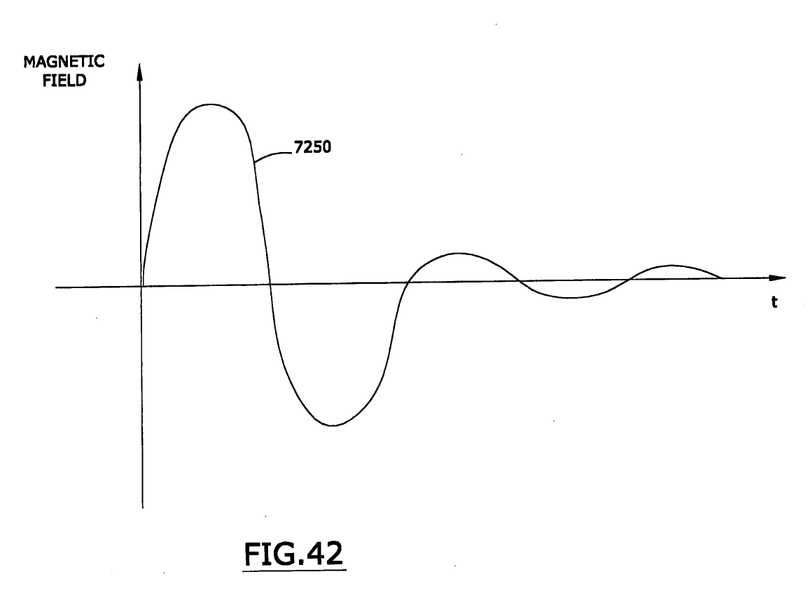

One typical demagnetizing process is schematically illustrated in FIG. 42. Referring to FIG. 42, it will be seen that one may dealign the clusters 7002 of magnetized object 7206 (see FIG. 41) by applying an exponentially decaying magnetic field 7250. These exponentially decaying magnetic fields are well known to those skilled in the art. Reference may be had, e.g., to U.S. Pat. No. 4,054,943 (circuit arrangement for the automatic deexcitation of a hysteresis motor); U.S. Pat. Nos. 4,202,932; 4,769,577 (cathode ray tube display with combined degauss and surge limiting circuit); U.S. Pat. No. 5,396,369 (degausser for magnetic stripe card transducing system); U.S. Pat. No. 5,350,403 (appraratus for charging living tissue with electrical pulses); U.S. Pat. Nos. 6,018,296; 6,545,580; 6,645,314; and the like; the entire disclosure of each of these United States patents is hereby incorporated by reference into this specification. Reference also may be had, e.g., to published U.S. patent applications 2002/0047767, 2003/0066537, 2004/0069379, and 2004/0074566; the entire disclosure of each of these U.S. patent applications is hereby incorporated by reference into this specification.

Referring to FIG. 42, the decaying alterning current field 7250 alternately aligns the clusters 7002 (see FIG. 41) first in one direction, and then in another direction, with continuously varying degrees of decaying intensity, thereby dealigning them. Thus, by the appropriate choice of external electromagnetic field(s), one can either increase or decrease the attraction between the nanomagnetic coating 7006 and the particles 7012.

A medical Device Comprised of Giant Magnetoresistive Material

FIG. 43 is a sectional view of an assembly 8000 comprised of a substrate 8002, a layer of nanomagnetic material 7006, and a layer of giant magnetoresistive material 8004.

The substrate 8002 may be any of the substates mentioned elsewhere in this specification. Thus, e.g., it may be a stent 8002, and/or a drug eluting stent 8002. In the embodiment depicted in FIG. 43, drug eluting polymer 8006 is disposed on surface 8008 of stent 8002.

Referring again to FIG. 43, it is preferred that the layer 8004 of giant magnetresistive material should preferably have a thickness 8010 of from about 200 nanometers to about 5 microns. In one embodiment, the thickness 8010 is from about 500 nanometers to about 1,500 nanometers.

It is preferred that the giant magnetostrictive material is comprised of at least 80 weight percent of a ceramic material. As used herein, the term ceramic material refers to any of a class of inorganic, nonmetallic products, and it includes metallic oxides, borides, carbides, or nitrides, and mixtures or compounds of such materials. See, e.g., page 54 of Loran S. O'Bannon's “Dictionary of Ceramic Science and Engineering” (Plenum Press, New York, N.Y., 1984).

By way of illustration and not limitation, suitable ceramic materials that possess giant magnetostrictive properties are described in various U.S. patents.

By way of illustration, one may use the magnetoresistive material described in claim 1 of U.S. Pat. No. 5,487,356, the entire disclosure of which is hereby incorporated by reference into this specification. Claim 1 of this patent describes: '1. A method of forming on a substrate a giant magnetoresistive (GMR) oxide material of the formula Laₓ A₁₋ₓ MnO₃ wherein: A is selected from the group consisting of barium, calcium, and strontium; and x is a number in the range of from 0.2 to 0.4, said method comprising the steps of: providing metalorganic compounds B, C and D in solution, as source reagent solution precursor for said GMR oxide material, wherein: B is a beta-diketonate or beta-ketoester adduct of lanthanum; C is a beta-diketonate or beta-ketoester adduct of A; and D is a beta-diketonate or beta-ketoester adduct of manganese; wherein: each of the B, C and D adducting ligands is the same as the others; and the molar concentration of B in said source reagent solution is from 0.002 to 0.2 moles/liter of solution; the molar concentration of C in said source reagent solution is from 0.002 to 0.2 moles/liter of solution; and the molar concentration of D in said source reagent solution is from 0.01 to 0.5 moles/liter of solution; vaporizing the source reagent solution precursor to form a multicomponent vapor comprising B, C, and D; directing the multicomponent vapor into a chemical vapor deposition reactor having a substrate disposed therein, for deposition of lanthanum, A, and manganese from the multicomponent vapor onto the substrate; maintaining a generally constant pressure in the chemical vapor deposition reactor of oxidizing gas therein, wherein the oxidizing gas is selected from the group consisting of oxygen and N₂ 0; and maintaining the substrate at a temperature in the range of 600° C. to 700° C. during said deposition of lanthanum, A, and manganese from the multicomponent vapor onto the substrate.” At columns 1-2 of this patent, “ . . . manganese oxide-based GMR materials were discussed.” It was disclosed that:The new manganese oxide-based GMR materials have formula: Lax A₁₋ₓ MnO₃ wherein A is selected from the group consisting of barium, calcium, and strontium, and x is a number in the range of from 0.2 to 0.4. The magnetic manganese oxide (La₁₋ₓ Aₓ)MnO₃, where A represents Ca, Sr, or Ba, has a perovskite-type crystal structure with ferromagnetic ordering in the a-b planes and antiferromagetic ordering along the c-axis below the Neel temperature. The ferromagnetically ordered Mn—O layers of the a-b planes are separated by a nonmagnetic La(A)-O monolayers. This spin structure is intrinsic, and is similar to that of the metallic multilayers described above which exhibited GMR.”

By way of yet further illustration, one may use the giant magnetoresistive material described in U.S. Pat. No. 5,776,359, the entire disclosure of which is hereby incorporated by reference into this specification. This patent claims “A giant magnetoresistive (GMR) cobalt oxide compound, said GMR cobalt oxide conpound having the formula: La_{y(1-x)}Ba_{y-x}CoO_{z} wherein y has a value ranging from about 1 to about 2; x has a value ranging from about 0.1 to about 0.9; and z has a value ranging from about 2 to about 4.

By way of yet further illustration, one may use one or more of the giant mangetoresistive materials described in U.S. Pat. Nos. 5,487,356; 5,587,943 (nonvolatile magnetoresistive memory); U.S. Pat. Nos. 5,639,547; 5,739,990; 5,776,359; 5,844,755 (giant magnetoresistive information recording medium); U.S. Pat. No. 5,859,754 (magnetoresistive transducer); U.S. Pat. No. 5,981,297 (biosensor); U.S. Pat. Nos. 5,995,338; 6,157,526 (giant magnetoresistive head); U.S. Pat. No. 6,282,068 (read head with improved GMR); U.S. Pat. No. 6,292,336 (giant magnetoresistive sensor element); U.S. Pat. Nos. 6,317,289; 6,327,122 (spin valve sensor); U.S. Pat. No. 6,353,519 (spin valve sensor); U.S. Pat. No. 6,512,660 (magnetoresistance read head); U.S. Pat. No. 6,559,401 (magnetic sandwich structure with large magnetoresistive effect); U.S. Pat. No. 6,621,666 (magnetoresistive-effect element having electrode layers); U.S. Pat. No. 6,621,667 (giant magnetoresistive sensor); U.S. Pat. No. 6,667,862 (magnetoresistive read head with magnetoresistive element); U.S. Pat. Nos. 6,669,787; 6,720,036 (giant magnetoresistive thin film); U.S. Pat. No. 6,669,983 (magnetic head with manetoresistive effect element); U.S. Pat. No. 6,686,068 (CPP GMR stacks); U.S. Pat. Nos. 6,762,481; 6,771,472 (GMR sensor); and the like. The entire disclosure of each of these U.S. patents is hereby incorporated by reference into this specification.

Referring again to FIG. 43, and in the preferred embodiment depicted therein, in one preferred embodiment at least about 90 weight percent of the giant magnetoresistive material is comprised of ceramic material. In one aspect of this embodiment, at least about 95 percent of the giant magnetoresistive material is comprised of ceramic material.

In one preferred embodiment, the giant magnetoresistive material used increases its resistance in response to both a direct current field of from about 0.5 to 3.0 Tesla (and preferably from about 1 to about 2 Tesla) and, simultaneously, a radiofrequency field with a frequency of from about 30 to about 128 megahertz (and preferably from about 45 to about 90 megahertz). Applicants have discovered that these combined fields, which correspond to the fields present in magnetic resonance imaging (MRI), increase the resistance of the preferred giant magnetoresistive materials by at least two orders of magnitude. Thus, if the resistance in the absence of both of such fields is, e.g., 10 ohms, the resistance in the presence of both of such fields is at least 1,000 ohms.

In one embodiment, the ceramic giant magnetoresistive material increases the surface resistance to eddy current flow by at least two orders of magnitude, and preferably by at least three orders of magnitude. The surface resistance to eddy current flow may be measured in accordance with conventional means.

U.S. Pat. No. 4,894,619, the entire disclosure of which is hereby incorporated by reference into this specification, discloses a method for detecting eddy currents. Claim 1 of this patent describes: “A method for improving the signal/interference ratio and material identification in the detection of metal objects by detecting and measuring eddy currents induced in such objects, comprising: using an impulse-type metal detector of the type providing a signal representing detected current having a transmitter and a receiver, causing a cut-off peak signal of brief duration to result from mutual inductance between the transmitter and the receiver by cutting off a flow of current to the transmitter, detecting said cut-off peak signal with the receiver, automatically compensating for mutual inductance between the transmitter and the receiver by using a compensation circuit, conducting measurements of signals caused by the presence of a metal body in at least two measuring sequences within the brief duration of said cut-off peak and in at least one measuring sequence after the cut-off peak and including comparing a signal measured during said cut-off peak with a predetermined alarm limit, comparing a signal measured after said cut-off peak with a predetermined alarm limit, and activating an alarm only when both said alarm limits are surpassed.”

U.S. Pat. No. 4,943,771, the entire disclosure of which is hereby incorporated by reference into this specification, describes and claims a differential eddy current sensor apparatus. Claim 1 of this patent describes: A differential eddy current sensor apparatus comprising in combination: means for oscillating, said oscillating means providing an exciting signal, means for measuring, said measuring means receiving said exciting signal, means for sensing, said sensing means operatively connected to said measuring means and comprising a first sensing coil and a second sensing coil, said first and second sensing coils being arranged in a parallel configuration and positioned a predetermined distance part, said target being positioned between said first and second sensing coils and spaced an equal distance away therefrom, a target operatively arranged with said sensing means to establish an impedance level which may be sensed by said sensing means, said impedance level being balanced when said sensing means is in a predetermined alignment with said target, said sensing means providing a differential signal to said measuring means when said alignment between said target and said sensing means is disturbed, a first means for supporting said target, said first supporting means operatively attached to a first mirror segment, and a second means for supporting said first and second sensing coils, said second supporting means operatively connected to a second mirror segment, and means for demodulating, said demodulating means operatively connected to said measuring means to receive said differential signal, said demodulating means amplifying and demodulating said differential signal to provide an analog output signal representative of the relative position of said mirror segments.”

U.S. Pat. No. 4,973,905, the entire disclosure of which is hereby incorporated by reference into this specification, discloses an eddy current measuring device with a flux return element. Claim 1 of this patent describes: “1. An eddy-current measuring device suitable for use as a tachometer, comprising a permanent magnet, a drive shaft, an eddy-current element, a pointer shaft extending beyond the drive shaft, and a return flux element of magnetic material; and wherein the permanent magnet has the shape of a circular disk and is positioned fixed for rotation on the drive shaft; the eddy-current element comprises electrically conductive nonmagnetic material, is formed as a circular disk positioned spaced apart from and alongside the permanent magnet disk, and is fixed for rotation on the pointer shaft; the return flux element is located alongside the eddy-current element on a side thereof opposite the magnet; the permanent magnet is magnetized axially; and said return element is disposed on and fixed to the eddy-current element for rotation with the eddy current element.”

Thus, e.g., reference also may be had to U.S. Pat. No. 6,762,604, the entrie disclosure of which is hereby incorporated by reference into this specification. The abstract of this patent describes: “A standalone eddy current monitoring system provides a thickness profile of a substrate sample by obtaining initial and terminating resistance and reactance measurements from the sample. Initial eddy current measurement values are obtained while an eddy current probe is positioned at an initial distance relative to the substrate sample, and terminating values are obtained while the eddy current probe is positioned at a modified distance relative to the sample. An intersecting line can be calculated using the initial and terminating resistance and reactance measurements. An intersecting point between a previously defined natural intercepting curve and the intersecting line may also be determined. A reactance voltage of the intersecting point may be located along a digital calibration curve to identify a closest-two of a plurality of calibration samples. The conductive top layer thickness of the substrate sample can then be determined by approximating a location, using linear or non-linear calculations, of the reactance voltage relative to the closest-two of the plurality of calibration samples.”

The aforementioned description of means for measuring eddy current 8032 is only illustrative, and many other eddy current measurement means will be apparent to those skilled in the art.

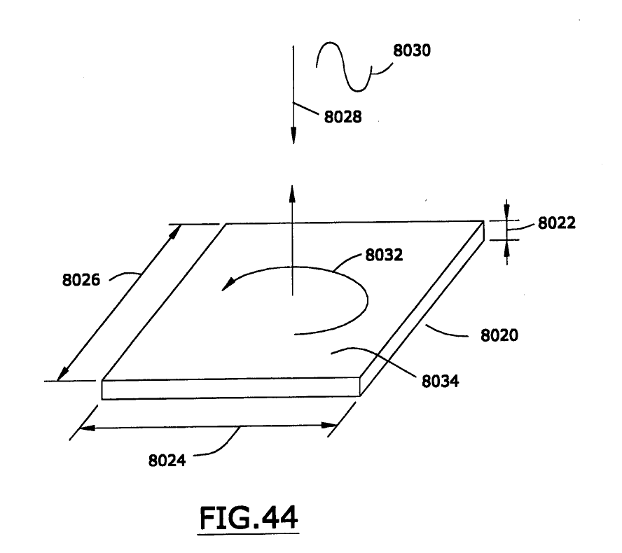

Thus, and referring to FIG. 44, a sheet 8020 of the giant magnetoresistive material to be tested is used. This test sheet 8020 typically has a thickness 8022 of 1 micron, a length 8024 of 1 centimeter, and a width 8026 of 1 centimeter.

Referring again to FIG. 44, a field 8028 is applied that is comprised of direct current field of 1.5 tesla, and also a radiofrequency field of 64 megahertz; this field is preferably similar to the fields present in magnetic resonance imaging (MRI). The altenating current portion 8030 of this combined field induces an eddy current 8032 in accordance with the well-known Faraday law of induction, and also with Lenz's law. This eddy current 8032 may be measured in accordance with conventional magnetic field measurement techniques. Thus, e.g., one or more of the surface eddy current measuring means described hereinabove by reference to FIG. 43.

Referring again to FIG. 44, the eddy currents 8032 that are being measured are the eddy currents on the surface 8034 of the sheet 8020. In one embodiment, the eddy currents 8032 so measured are less than about 10 microamperes in the presence of the specified combined field 8028, and more preferably less than about 5 microamperes. In one embodiment, the eddy currents are less than about 1 microampere.

FIG. 45 is a schematic representation of a portion of a stent 8100 comprised of struts 8102, 8104, and 8106, each of which is comprised of the giant magnetresistive material 8004 and nanomagnetic material 7006. In the presence of the combined field 8028, substantially no eddy currents are created due to the presence of such giant magnetoresistive material; and, thus, no imaging distortion is created on account of such eddy currents. The nanomagnetic material 7006 present in each of such struts helps lead the radio frequency portion of combined field 8028 through openings 8108 and 8110 so that they can interact with a biolgocial object to be imaged, such as, e.g., plaque 8112. The nanomagnetic material is chosen with an appropriate susceptibility such that the radiofrequency field can be led into and through openings 8108 and 8110 (in the direction, e.g., of arrows 8118 and 8120), and the signals created by the interaction of such radio frequency field and the biological object 8112 can also pass back through openings 8108 and 8110 (in the direction, e.g., of arrows 8114 and 8116).

As will be apparent, and referring again to FIGS. 44 and 45, the use of both the nanomagnetic coating 7006 and the giant magnetoresistive coating 8004 renders the coated stent transparent to the applied MRI field 8028 such that it only presents a desired image for the biological object 8112.

Cancellation of the Positive Susceptibility of a Nitinol Stent

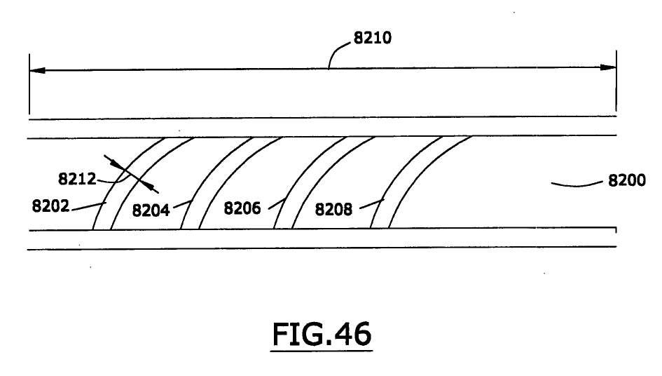

In one preferred embodiment, illustrated in FIG. 46, a stent 8200 constructed form Nitinol is comprised of struts 8202, 8204, 8206, and 8208 coated with a layer of elemental bismuth. As is known to those skilled in the art, Nitinol is a paramagnetic alloy that was developed by the Naval Ordnance Laboratory; it is an intermetallic compound of nickel and titanium. See, e.g., page 552 of George S. Brady et al.'s “Materials Handbook,” Thirteenth Edition (McGraw-Hill Company, New York, N.Y., 1991).

Referring again to FIG. 46, and to the preferred embodiment depicted therein, the stent 8200 is preferably cylindrical with a diameter (not shown) of less than 1 centimeter and a length 8210 of about 3 centimeters. Each strut, such as strut 8202, is preferably arcuate, having an effective diameter 8212 of less than about 1 millimeter.

As is known to those skilled in the art, the magnetic permeability of the Nitinol material is about 1.003; and its susceptibility is about 0.03 centimeter-grams-seconds (cgs). In order to nullify the susceptibility, one can introduce a diamagnetic material, such as bismuth, that has a negative susceptibility. In one embodiment, a bismuth coating with a thickness of form about 10 to about 20 microns is deposited upon each of the struts 8202.

Thus, and as will be apparent from the disussions presented in other parts of this specification, the susceptibility for these struts 8202 becomes substantially zero, whereby there is no substantial direct current (d.c.) susceptibility distortion in the MRI field. As used herein, the term “substantially zero” refers to a net susceptibility of from about 0.9 to about 1.1.

As will be apparent, when applicant's nanomagnetic coating 7006, and/or applicants giant magnetoresistive coating 8004, are added to such stent, the amount and type of the coatings are chosen such that the net susceptibility for the struts is still preferably substantially zero,

As will be also apparent, susceptibility varies with both direct current and alternating current. It is desired that, with the composite coating described hereinabove, the susceptibility at a direct current field of about 1.5 Tesla (which is also the slope of the plot of magnetization versus the applied magnetic field), should preferably be from about 0.9 to about 1.1.

A Medical Device with Improved Drug Delivery Capabilities

In this section of the specification, applicants will describe a medical device with improved drug delivery capabilities. This medical device is similar to the medical device disclosed in published U.S. patent application 2004/0030379, the entire disclosure of which is hereby incorporated by reference into this specification. However, because applicants use an improved form of magnetic particles in the device, applicants device provides superior magnetic performance and, additionally, superior MRI imageability.

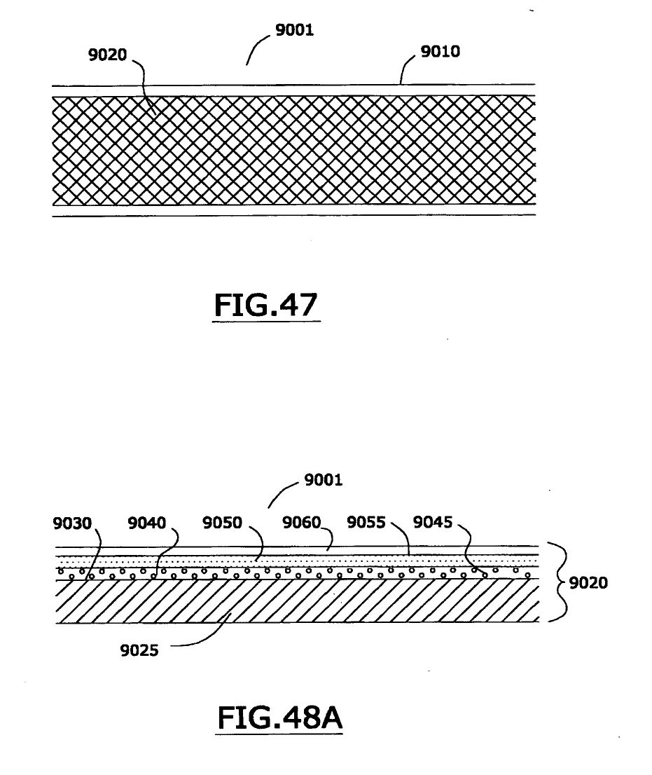

The medical system described in this section of the specification is preferably a stent 9010 (see FIG. 47) comprised of wire like struts 9020 (also see FIG. 47). As is disclosed in paragraph 22 of published U.S. patent application 2004/0030379, “The system of the present invention comprises (1) a medical device having a coating containing a biologically active material, and (2) a source of electromagnetic energy or a source for generating an electromagnetic field. The present invention can facilitate and/or modulate the delivery of the biologically active material from the medical device. The release of the biologically active material from the medical device is facilitated or modulated by the electromagnetic energy source or field. To utilize the system of the present invention, the practitioner may implant the coated medical device using regular procedures. After implantation, the patient is exposed to an extracorporal or external electromagnetic energy source or field to facilitate the release of the biologically active material from the medical device. The delivery of the biologically active material is on-demand, i.e., the material is not delivered or released from the medical device until a practitioner determines that the patient is in need of the biologically active material. The coating of the medical device of the present invention further comprises particles comprising a magnetic material, i.e., magnetic particles . . . .”

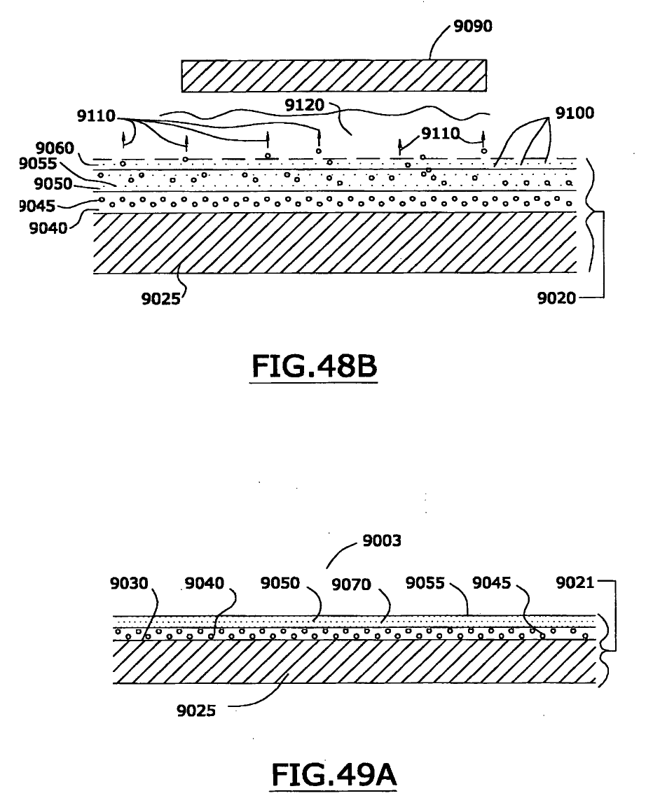

One embodiment of the medical device 9001 (see FIG. 47) is illustrated in FIG. 48A, which shows a cross-sectional view of a coated strut 9020 of the stent. In the embodiment depicted in FIG. 48A, the coated strut 9020 comprises a strut 9025 having a surface 9030. The coated strut 9020 has a composite coating that comprises a first coating layer 9040 that contains a biologically active material 9045; in one embodiment, this first coating layer 9040 also also contains polymeric material.

Referring again to FIG. 48A, a second coating layer 9050 comprising nanomagnetic particles 9055 is disposed over the first coating layer 40. This second coating layer 9055, in one embodiment, also includes polymeric material.

Referring again to FIG. 48A, and in the preferred embodiment depicted, a third coating layer or sealing layer 9060 is disposed on top of the second coating layer 9050.

FIG. 48B is similar to FIG. 2B of U.S. published patent application 2004/0030379; and it illustrates the effect of exposing a patient (not shown), who is implanted with a stent having struts 9020 shown in FIG. 48A, to an electromagnetic energy source or field 9090. When such a field 9090 is applied, the magnetic particles 9055 move out of the second coating layer 9050 in the direction of upward arrow 9110. This movement disrupts the sealing layer 9160 and forms channels 9100 in such sealing layer 9060.

Referring again to FIG. 48B, it will be seen that the size of the channels 9100 formed generally depends on the size of the magnetic particles 9055 used. The biologically active material 9045 can then be released from the coating through the disrupted sealing layer 9060 into the surrounding tissue 9120. The duration of exposure to the field and the strength of the electromagnetic field 9090 determine the rate of delivery of the biologically active material 9045.

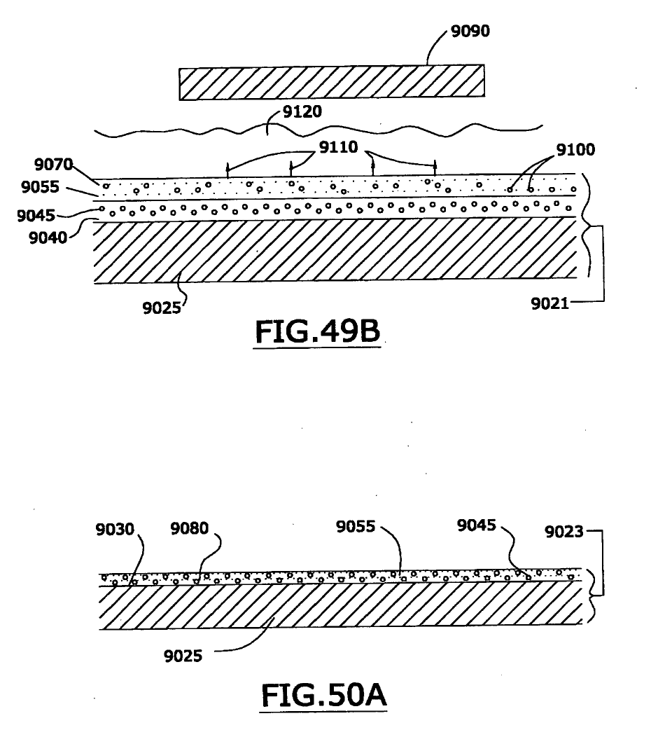

FIG. 49A illustrates another coated stent 9003; this Figure is similar to Fugure 3A of U.S. published patent application 2004/0030379. Referring to FIG. 49A, and in the preferred embodiment depicted therein, it will be seen that, in this embodiment, the coated strut 9021 contains a coating comprised of a first coating layer 9040 comprising a biologically active material 9045 and preferably a polymeric material disposed over the surface 9030 of the strut 25. A second coating layer or sealing layer 9070 comprising magnetic particles 9055 and a polymeric material is disposed on top of the first coating layer 9040.

FIG. 49B illustrates the effect of exposing a patient (not shown) who is implanted with a stent having struts 9021 shown in FIG. 49A to an electromagnetic field 9090; this Figure is similar to FIG. 3B of U.S. published patent application 2004/0030379. Referring to FIG. 49B, when such a field 9090 is applied, the magnetic particles 9055 move through the sealing layer 9070 as shown by the upward arrow 9110, and they create channels 9100 in the sealing layer 9070. The biologically active material 9045 in the underlying first coating layer 9040 is allowed to travel through the channels 9100 in the sealing layer 9070 and be released to the surrounding tissue 9120. Since the biologically active material 9045 is in a separate first coating layer 9040 and must migrate through the second coating layer or the sealing layer 9070, the release of the biologically active material 9045 is controlled after formation of the channels 9100.

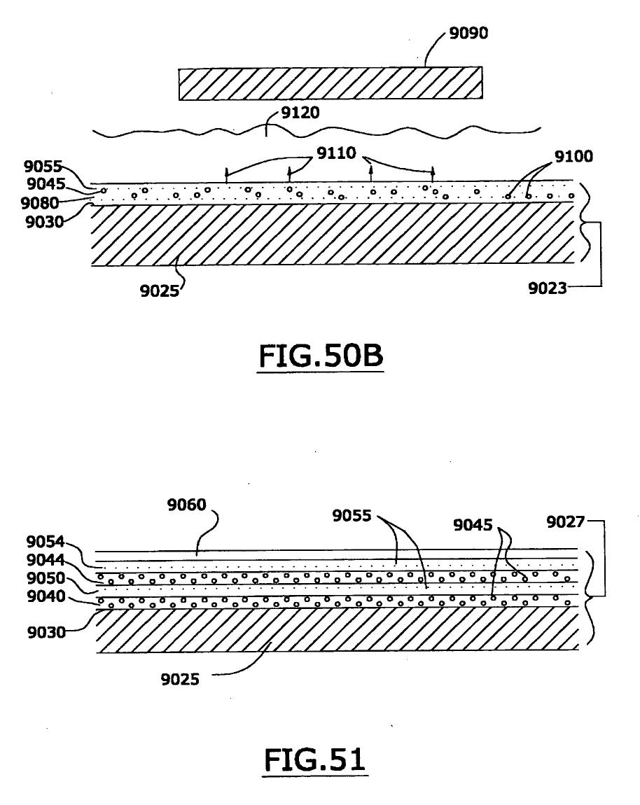

FIG. 50A is similar to FIG. 4A of published U.S. patent application 2004/0030379, and it shows another embodiment of a coated stent strut 9023. In this embodiment, the coating comprises a coating layer 9080 comprising a biologically active material 9045, magnetic particles 9055, and a polymeric material.

FIG. 50B, which is similar to FIG. 4B of published U.S. patent application 2004/0030379, illustrates the effect of exposing a patient (not shown) who is implanted with a stent having struts 9023 to an electromagnetic field 9090. The field 9090 is applied, the magnetic particles 905555 move through the layer 9080 as shown by the arrow 9110 and create channels in the coating layer 9080. The biologically active material 9045 can then be released to the surrounding tissue 9120.

In another embodiment, and referring to FIGS. 47 and 51, the medical device 9001 of the present invention may be a stent having struts coated with a coating comprising more than one coating layer containing a magnetic material. FIG. 51 illustrates such a coated strut 9027. The coating comprises a first coating layer 9040 containing a polymeric material and a biologically active material 9045 which is disposed on the surface 9030 of a strut 9025. A second coating layer 9050 comprising a polymeric material and magnetic particles 9055 is disposed over the first coating layer 9040. A third coating layer 9044 comprising a polymeric material and a biologically active material 9045 is disposed over the second coating layer 9050. A fourth coating layer 9054 comprising a polymeric material and magnetic particles 9055 is disposed over this third layer 9044. Finally a sealing layer 9060 of a polymeric material is disposed over the fourth coating layer 9054. The permeability of the coating layers may be different from layer to layer so that the release of the biologically active material from each layer can differ. Also, the magnetic susceptibility of the magnetic particles may differ from layer to layer. The magnetic susceptibility may be varied using different concentrations or percentages of magnetic particles in the coating layers. The magnetic susceptibility of the magnetic particles may also be varied by changing the size and type of material used for the magnetic particles. When the magnetic susceptibility of the magnetic particles differs from layer to layer, different excitation intensity and/or frequency are required to activate the magnetic particles in each layer.

Referring again to FIG. 51, (and also to paragraph 27 at page 3 of published U.S. patent application 2004/0030379), the nanomagnetic particles preferably used in the embodiment depicted in FIG. 51 may be coated with a biologically active material and then incorporated into a coating for the medical device. In one embodiment, the biologically active material is a nucleic acid molecule. The nucleic acid coated nanomagnetic magnetic particles may be formed by painting, dipping, or spraying the magnetic particles with a solution comprising the nucleic acid. The nucleic acid molecules may adhere to the nanomagnetic particles via adsorption. Also the nucleic acid molecules may be linked to the magnetic particles chemically, via linking agents, covalent bonds, or chemical groups that have affinity for charged molecules. Application of an external electromagnetic field can cause the adhesion between the biologically active material and the magnetic particle to break, thereby allowing for release of the biologically active material.

In another embodiment, and referring to such FIGS. 47-51, the magnetic particles may be molded into or coated onto a non-metallic medical device, including a bio-absorb able medical device. The magnetic properties of the preferred nanomagnetic particles allow the non-metallic implant to be extracorporally imaged, vibrated, or moved. In specific embodiments, the nanomagnetic particles are painted, dipped or sprayed onto the outer surface of the device. The naomagnetic particles may also be suspended in a curable coating, such as a UV curable epoxy, or they may be electrostatically sprayed onto the medical device and subsequently coated with a UV or heat curable polymeric material.

Additionally, and in some embodiments, the movement of the magnetic particles that occurs when the patient implanted with the coated device is exposed to an external electromagnetic field, releases mechanical energy into the surrounding tissue in which the medical device is implanted and triggers histamine production by the surrounding tissues. The histamine has a protective effect in preventing the formation of scar tissues in the vicinity at which the medical device is implanted.

In one embodiment, the movement of the preferred nanomagnetic particles creates a sufficient amount of heat to kill cells by hyperthermia. This embodiment is described elsewhere in this specification, wherein nanomagnetic particles with specified Curie temperatures that preferentially kill cancer cells when heated are described.

In one preferred embodiment, the application of the external electromagnetic field 9090 activates the biologically active material in the coating of the medical device. A biologically active material that may be used in this embodiment may be a thermally sensitive substance that is coupled to nitric oxide, e.g., nitric oxide adducts, which prevent and/or treat adverse effects associated with use of a medical device in a patient, such as restenosis and damaged blood vessel surface. The nitric oxide is attached to a carrier molecule and suspended in the polymer of the coating, but it is only biologically active after a bond breaks, thereby releasing the smaller nitric oxide molecule in the polymer and eluting into the surrounding tissue. Typical nitric oxide adducts include, e.g., nitroglycerin, sodium nitroprusside, S-nitroso-proteins, S-nitroso-thiols, long carbon-chain lipophilic S-nitrosothiols, S-nitrosodithiols, iron-nitrosyl compounds, thionitrates, thionitrites, sydnonimines, furoxans, organic nitrates, and nitrosated amino acids, preferably mono- or poly-nitrosylated proteins, particularly polynitrosated albumin or polymers or aggregates thereof. The albumin is preferably human or bovine, including humanized bovine serum albumin. Such nitric oxide adducts are disclosed in U.S. Pat. No. 6,087,479 to Stamler et al., the entire disclosure of which is incorporated herein by reference into this specification.

In one embodiment, the application of the electromagnetic field 9090 effects a chemical change in the polymer coating, thereby allowing for faster release of the biologically active material from the coating.

Paragraphs 32-35 of published U.S. patent application 2004/0030379 are applicable to the devices of the instant invention. They are presented herein in their entireties.

“B. Drug Release Modulation Employing a Mechanical Vibrational Energy Source”

“Another embodiment of the present invention is a system for delivering a biologically active material to a body of a patient that comprises a mechanical vibrational energy source and an insertable medical device comprising a coating containing the biologically active material. The coating can optionally contain magnetic particles. After the device is implanted in a patient, the biologically active material can be delivered to the patient on-demand or when the material is needed by the patient. To deliver the biologically active material, the patient is exposed to an extracorporal or external mechanical vibrational energy source. The mechanical vibrational energy source includes various sources which cause vibration such as sonic or ultrasonic energy. Exposure to such energy source causes disruption in the coating that allows for the biologically active material to be released from the coating and delivered to body tissue.”

“Moreover, in certain embodiments, the biologically active material contained in the coating of the medical device is in a modified form. The modified biologically active material has a chemical moiety bound to the biologically active material. The chemical bond between the moiety and the biologically active material is broken by the mechanical vibrational energy. Since the biologically active material is generally smaller than the modified biologically active material, it is more easily released from the coating. Examples of such modified biologically active materials include the nitric oxide adducts described above.”

“In another embodiment, the coating comprises at least a coating layer containing a polymeric material whose structural properties are changed by mechanical vibrational energy. Such change facilitates release of the biologically active material which is contained in the same coating layer or another coating layer.”

Paragraphs 36, 37, 38, 39, 40, and 41 of published U.S. patent application 2004/0030379 are also applicable to the medical devices of this invention. They are presented below in their entireties.

“C. Materials Suitable for the Invention 1. Suitable Medical Devices”

“The medical devices of the present invention are insertable into the body of a patient. Namely, at least a portion of such medical devices may be temporarily inserted into or semi-permanently or permanently implanted in the body of a patient. Preferably, the medical devices of the present invention comprise a tubular portion which is insertable into the body of a patient. The tubular portion of the medical device need not to be completely cylindrical. For instance, the cross-section of the tubular portion can be any shape, such as rectangle, a triangle, etc., not just a circle.”

“The medical devices suitable for the present invention include, but are not limited to, stents, surgical staples, catheters, such as central venous catheters and arterial catheters, guidewires, balloons, filters (e.g., vena cava filters), cannulas, cardiac pacemaker leads or lead tips, cardiac defibrillator leads or lead tips, implantable vascular access ports, stent grafts, vascular grafts or other grafts, interluminal paving system, intra-aortic balloon pumps, heart valves, cardiovascular sutures, total artificial hearts and ventricular assist pumps.”

“Medical devices which are particularly suitable for the present invention include any kind of stent for medical purposes, which are known to the skilled artisan. Suitable stents include, for example, vascular stents such as self-expanding stents and balloon expandable stents. Examples of self-expanding stents useful in the present invention are illustrated in U.S. Pat. Nos. 4,655,771 and 4,954,126 issued to Walisten and U.S. Pat. No. 5,061,275 issued to Wallsten et al. Examples of appropriate balloon-expandable stents are shown in U.S. Pat. No. 4,733,665 issued to Palmaz, U.S. Pat. No. 4,800,882 issued to Gianturco, U.S. Pat. No. 4,886,062 issued to Wiktor and U.S. Pat. No. 5,449,373 issued to Pinchasik et al. A bifurcated stent is also included among the medical devices suitable for the present invention.”

“The medical devices suitable for the present invention may be fabricated from polymeric and/or metallic materials. Examples of such polymeric materials include polyurethane and its copolymers, silicone and its copolymers, ethylene vinyl-acetate, poly(ethylene terephthalate), thermoplastic elastomer, polyvinyl chloride, polyolephines, cellulosics, polyamides, polyesters, polysulfones, polytetrafluoroethylenes, acrylonitrile butadiene styrene copolymers, acrylics, polyactic acid, polyclycolic acid, polycaprolactone, polyacetal, poly(lactic acid), polylactic acid-polyethylene oxide copolymers, polycarbonate cellulose, collagen and chitins. Examples of suitable metallic materials include metals and alloys based on titanium (e.g., nitinol, nickel titanium alloys, thermo-memory alloy materials), stainless steel, platinum, tantalum, nickel-chrome, certain cobalt alloys including cobalt-chromium-nickel alloys (e.g., Elgiloy® and Phynox®) and gold/platinum alloy. Metallic materials also include clad composite filaments, such as those disclosed in WO 94/16646.”

Paragraphs 42-47 of published U.S. patent application 2004/0030379 describes the magnetic particles used in the device of such application. In applicants' preferred device, the magnetic particles of such device are replaced with certain nanomagnetic particles described elsewhere in this specification These nanomangetic particles preferably have the properties described below.

The nanomagnetic particles are usually in to form of a coating a nanomagnetic material comprised of such particles. An assembly comprised of a device, wherein said device comprises a substrate and, disposed over such substrate, nanomagnetic material and magetoresistive material, wherein the nanomagnetic material has a saturation magentization of from about 2 to about 3000 electromagnetic units per cubic centimeter. The nanomagnetic particles generally have an average particle size of less than about 100 nanometers, wherein the average coherence length between adjacent nanomagnetic particles is less than 100 nanometers.

In one embodiment, the nanomagnetic material has an average particle size of less than about 20 nanometers and a phase transition temperature of less than about 200 degrees Celsius.

In one embodiment, the average particle size of such nanomagnetic particles is less than about 15 nanometers. In another embodiment, the nanomagentic material has a saturation magnetization of at least 2,000 electromagnetic units per cubic centimeter.

In yet another embodiment, the nanomagnetic material has a saturation magnetization of at least 2,500 electromagnetic units per cubic centimeter.

In yet another embodiment, the nanomagnetic, the particles of nanomagnetic material have a squareness of from about 0.05 to about 1.0.

In yet another embodiment, the nanomagnetic, the particles of nanomagnetic material are at least triatomic, being comprised of a first distinct atom, a second distinct atom, and a third distinct atom. In one aspect of this embodiment, the first distinct atom is an atom selected from the group consisting of atoms of actinium, americium, berkelium, californium, cerium, chromium, cobalt, curium, dysprosium, einsteinium, erbium, europium, fermium, gadolinium, holmium, iron, lanthanum, lawrencium, lutetium, manganese, mendelevium, nickel, neodymium, neptunium, nobelium, plutonium, praseodymium, promethium, protactinium, samarium, terbium, thorium, thulium, uranium, and ytterbium. In another aspect of this embodiment, the distinct atom is a cobalt atom.

In yet another embodiment, the particles of nanomagnetic material are comprised of atoms of cobalt and atoms of iron.

In yet another embodiment, such first distinct atom is a radioactive cobalt atom.

In yet another embodiment, the particles of nanomagnetic material are comprised of a said first distinct atom, said second distinct atom, said third distinct atom, and a fourth distinct atom. In one aspect of this embodiment, the particles of nanomagnetic material are comprised of a fifth distinct atom.

In yet another embodiment, such particles of nanomagnetic material have a sqareness of from about 0.1 to about 0.9. In one aspect of this embodiment, such particles of nanomagnetic material have a squarenesss is from about 0.2 to about 0.8.

In yet another embodiment, the nanomagnetic particles have an average size of less of less than about 3 nanometers. In yet another embodiment, the nanomagnetic particles have an average size of less than about 15 nanometers. In yet another embodiment, the nanomagnetic particles have an average size is less than about 11 nanometers.

In yet another embodiment, the nanomagnetic particles have a phase transition temperature of less than 46 degrees Celsius. In yet another embodiment, the nanomagnetic particles have a a phase transition temperature of less than about 50 degrees Celsius.

In yet another embodiment, the nanomagnetic material has a coercive force of from about 0.1 to about 10 Oersteds.

In yet another embodiment, the nanomagnetic particles have a relative magnetic permeability of from about 1.5 to about 2,000.

In yet another embodiment, the nanomagnetic particles have a saturation magnetization of at least 100 electromagnetic units per cubic centimeter. In one aspect of this embodiment, the particles of nanomagnetic material have a saturation magnetization of at least about 200 electromagnetic units (emu) per cubic centimeter. In yet another aspect of this embodiment, the particles of nanomagnetic material have a saturation magnetization of at least about 1,000 electromagnetic units per cubic centimeter.

In yet another embodiment, the nanomagnetic particles have a coercive force of from about 0.01 to about 5,000 Oersteds. In one aspect of this embodiment, such particles of nanomagnetic material have a coercive force of from about 0.01 to about 3,000 Oersteds.

In yet another embodiment, the nanomagnetic particles have a relative magnetic permeability of from about 1 to about 500,000. In one aspect of this embodiment, such particles have a relative magnetic permeability of from about 1.5 to about 260,000.

In yet another embodiment, the nanomagnetic particles have a mass density of at least about 0.001 grams per cubic centimeter. In one aspect of this embodiment, such particles of nanomagnetic material have a mass density of at least about 1 gram per cubic centimeter. In another aspect of this embodiment, such particles of nanomagnetic material have a mass density of at least about 3 grams per cubic centimeter. In yet another aspect of this embodiment, such particles of nanomagnetic material have a mass density of at least about 4 grams per cubic centimeter.

In yet another embodiment, the second distinct atom of such nanomagnetic particles has a relative magnetic permeability of about 1.0. In one aspect of this embodiment, such second distinct atom is an atom selected from the group consisting of aluminum, antimony, barium, beryllium, boron, bismuth, calcium, gallium, germanium, gold, indium, lead, magnesium, palladium, platinum, silicon, silver, strontium, tantalum, tin, titanium, tungsten, yttrium, zirconium, magnesium, and zinc.

In yet another embodiment, the nanomagnetic particles are comprised of a third distinct atom that is an atom selected from the group consisting of argon, bromine, carbon, chlorine, fluorine, helium, helium, hydrogen, iodine, krypton, oxygen, neon, nitrogen, phosphorus, sulfur, and xenon. In one aspect of this embodiment, the third distinct atom is nitrogen.

In yet another embodiment, the nanomagnetic particles are represented by the formula AₓB_{y}C_{z}, wherein A is said first distinct atom, B is said second distinct atom, C is said third distinct atom, and x+y+z is equal to 1. In one aspect of this embodiment, such nanomagnetic particles are comprised of atoms of oxygen. In another aspect of this embodiment, the nanomagnetic particles are comprised of atoms of iro which optionally me be radioactive. In another aspect of this embodiment, such nanomagnetic particles are comprised of atoms of cobalt which, optinally, may be radioactive.

In yet another embodiment, the particles of nanomagnetic material are present in the form of a coating with a thickness of from about 400 to about 2000 nanometers. In one aspect of this embodiment, the coating has a thickness of from about 600 to about 1200 nanometers. In another aspect of this embodiment, the coating has a morphological density of at least about 98 percent, preferably at least about 99 percent, and more preferably at least about 99.5 percent. In another aspect of this embodiment, such coating has an average surface roughness of less than about 100 nanometers, and preferably of less than about 10 nanometers. In another aspect of this embodiment, such coating is biocompatiable. In another aspect of this embodiment, such coating is is hydrophobic. In yet another aspect of this embodiment, such coating is hydrophilic.

Paragraphs 48, through 72 of published U.S. patent application 2004/0030379 describe biologically active material that may be used in the device of this invention. This paragraphs are presented below in their entireties.

“3. Biologically Active Material”

“The term ‘biologically active material’ encompasses therapeutic agents, such as drugs, and also genetic materials and biological materials. The genetic materials mean DNA or RNA, including, without limitation, of DNA/RNA encoding a useful protein stated below, anti-sense DNA/RNA, intended to be inserted into a human body including viral vectors and non-viral vectors. Examples of DNA suitable for the present invention include DNA encoding . . . anti-sense RNA . . . tRNA or rRNA to replace defective or deficient endogenous molecules . . . angiogenic factors including growth factors, such as acidic and basic fibroblast growth factors, vascular endothelial growth factor, epidermal growth factor, transforming growth factor α and β, platelet-derived endothelial growth factor, plateletderived growth factor, tumor necrosis factor α, hepatocyte growth factor and insulin like growth factor . . . cell cycle inhibitors including CD inhibitors . . . thymidine kinase (“TK”) and other agents useful for interfering with cell proliferation, and . . . the family of bone morphogenic proteins (“BMP's”) as explained below. Viral vectors include adenoviruses, gutted adenoviruses, adeno-associated virus, retroviruses, alpha virus (Semliki Forest, Sindbis, etc.), lentiviruses, herpes simplex virus, ex vivo modified cells (e.g., stem cells, fibroblasts, myoblasts, satellite cells, pericytes, cardiomyocytes, sketetal myocytes, macrophage), replication competent viruses (e.g., ONYX-015), and hybrid vectors. Non-viral vectors include artificial chromosomes and mini-chromosomes, plasmid DNA vectors (e.g., pCOR), cationic polymers (e.g., polyethyleneimine, polyethyleneimine (PEI)) graft copolymers (e.g., polyether-PEI and polyethylene oxide-PEI), neutral polymers PVP, SP1017 (SUPRATEK), lipids or lipoplexes, nanoparticles and microparticles with and without targeting sequences such as the protein transduction domain (PTD).”

“The biological materials include cells, yeasts, bacteria, proteins, peptides, cytokines and hormones. Examples for peptides and proteins include growth factors (FGF, FGF-1, FGF-2, VEGF, Endotherial Mitogenic Growth Factors, and epidermal growth factors, transforming growth factor α and β, platelet derived endothelial growth factor, platelet derived growth factor, tumor necrosis factor α, hepatocyte growth factor and insulin like growth factor), transcription factors, proteinkinases, CD inhibitors, thymidine kinase, and bone morphogenic proteins (BMP's), such as BMP-2, BMP-3, BMP-4, BMP-5, BMP-6 (Vgr-1), BMP-7 (OP-1), BMP-8. BMP-9, BMP-10, BMP-11, BMP-12, BMP-13, BMP-14, BMP-15, and BMP-16. Currently preferred BMP's are BMP-2, BMP-3, BMP-4, BMP-5, BMP-6, BMP-7. Alternatively or in addition, molecules capable of inducing an upstream or downstream effect of a BMP can be provided. Such molecules include any of the “hedgehog” proteins, or the DNA's encoding them. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include whole bone marrow, bone marrow derived mono-nuclear cells, progenitor cells (e.g., endothelial progentitor cells) stem cells (e.g., mesenchymal, hematopoietic, neuronal), pluripotent stem cells, fibroblasts, macrophage, and satellite cells.”

“Biologically active material also includes non-genetic therapeutic agents, such as: . . . anti-thrombogenic agents such as heparin, heparin derivatives, urokinase, and PPack (dextrophenylalanine proline arginine chloromethylketone); . . . anti-proliferative agents such as enoxaprin, angiopeptin, or monoclonal antibodies capable of blocking smooth muscle cell proliferation, hirudin, and acetylsalicylic acid, amlodipine and doxazosin; . . . anti-inflammatory agents such as glucocorticoids, betamethasone, dexamethasone, prednisolone, corticosterone, budesonide, estrogen, sulfasalazine, and mesalamine; . . . immunosuppressants such as sirolimus (RAPAMYCIN), tacrolimus, everolimus and dexamethasone, . . . antineoplastic/antiproliferative/anti-miotic agents such as paclitaxel, 5-fluorouracil, cisplatin, vinblastine, vincristine, epothilones, methotrexate, azathioprine, halofuginone, adriamycin, actinomycin and mutamycin; cladribine; endostatin, angiostatin and thymidine kinase inhibitors, and its analogs or derivatives; . . . anesthetic agents such as lidocaine, bupivacaine, and ropivacaine; . . . anti-coagulants such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors and tick antiplatelet peptides; . . . vascular cell growth promotors such as growth factors, Vascular Endothelial Growth Factors (FEGF, all types including VEGF-2), growth factor receptors, transcriptional activators, and translational promotors; vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; . . . cholesterol-lowering agents; vasodilating agents; and agents which interfere with endogenous vasoactive mechanisms; . . . anti-oxidants, such as probucol; . . . antibiotic agents, such as penicillin, cefoxitin, oxacillin, tobranycin . . . angiogenic substances, such as acidic and basic fibrobrast growth factors, estrogen including estradiol (E2), estriol (E3) and 17-Beta Estradiol; and . . . drugs for heart failure, such as digoxin, beta-blockers, angiotensin-converting enzyme (ACE) inhibitors including captopril and enalopril.”

“Also, the biologically active materials of the present invention include trans-retinoic acid and nitric oxide adducts. A biologically active material may be encapsulated in micro-capsules by the known methods.”

Paragraphs 73 through 82 of published U.S. patent application 1004/0030379 describe coating compositons that may be used in the device of the instant invention; and they are reproduced in their entireties below.

“4. Coating Compositions . . . The coating compositions suitable for the present invention can be applied by any method to a surface of a medical device to form a coating. Examples of such methods are painting, spraying, dipping, rolling, electrostatic deposition and all modern chemical ways of immobilization of bio-molecules to surfaces.”

“The coating composition used in the present invention may be a solution or a suspension of a polymeric material and/or a biologically active material and/or magnetic particles in an aqueous or organic solvent suitable for the medical device which is known to the skilled artisan. A slurry, wherein the solid portion of the suspension is comparatively large, can also be used as a coating composition for the present invention. Such coating composition may be applied to a surface, and the solvent may be evaporated, and optionally heat or ultraviolet (UV) cured.”

“The solvents used to prepare coating compositions include ones which can dissolve the polymeric material into solution and do not alter or adversely impact the therapeutic properties of the biologically active material employed. For example, useful solvents for silicone include tetrahydrofuran (THF), chloroform, toluene, acetone, isooctane, 1,1,1-trichloroethane, dichloromethane, and mixture thereof.”

“A coating of a medical device of the present invention may consist of various combinations of coating layers. For example, the first layer disposed over the surface of the medical device can contain a polymeric material and a first biologically active material. The second coating layer, that is disposed over the first coating layer, contains magnetic particles and optionally a polymeric material. The second coating layer protects the biologically active material in the first coating layer from exposure during implantation and prior to delivery. Preferably, the second coating layer is substantially free of a biologically active material.”

“Another layer, i.e. sealing layer, which is free of magnetic particles, can be provided over the second coating layer. Further, there may be another coating layer containing a second biologically active material disposed over the second coating layer. The first and second biologically active materials may be identical or different. When the first and second biologically active material are identical, the concentration in each layer may be different. The layer containing the second biologically active material may be covered with yet another coating layer containing magnetic particles. The magnetic particles in two different layers may have an identical or a different average particle size and/or an identical or a different concentrations. The average particle size and concentration can be varied to obtain a desired release profile of the biologically active material. In addition, the skilled artisan can choose other combinations of those coating layers.”

“Alternatively, the coating of a medical device of the present invention may comprise a layer containing both a biologically active material and magnetic particles. For example, the first coating layer may contain the biologically active material and magnetic particles, and the second coating layer may contain magnetic particles and be substantially free of a biologically active material. In such embodiment, the average particle size of the magnetic particles in the first coating layer may be different than the average particle size of the magnetic particles in the second coating layer. In addition, the concentration of the magnetic particles in the first coating layer may be different than the concentration of the magnetic particles in the second coating layer. Also, the magnetic susceptibility of the magnetic particles in the first coating layer may be different than the magnetic susceptibility of the magnetic particles in the second coating layer.”

“The polymeric material should be a material that is biocompatible and avoids irritation to body tissue. Examples of the polymeric materials used in the coating composition of the present invention include, but not limited to, polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate, styrene-isobutylene copolymers and blends and copolymers thereof. Also, other examples of such polymers include polyurethane (BAYHDROL®, etc.) fibrin, collagen and derivatives thereof, polysaccharides such as celluloses, starches, dextrans, alginates and derivatives, hyaluronic acid, and squalene. Further examples of the polymeric materials used in the coating composition of the present invention include other polymers which can be used include ones that can be dissolved and cured or polymerized on the medical device or polymers having relatively low melting points that can be blended with biologically active materials. Additional suitable polymers include, thermoplastic elastomers in general, polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid-polyethylene oxide copolymers, EPDM (etylene-propylene-diene) rubbers, fluorosilicones, polyethylene glycol, polysaccharides, phospholipids, and combinations of the foregoing. Preferred is polyacrylic acid, available as HYDROPLUS® (Boston Scientific Corporation, Natick, Mass.), and described in U.S. Pat. No. 5,091,205, the disclosure of which is hereby incorporated herein by reference. In a most preferred embodiment of the invention, the polymer is a copolymer of polylactic acid and polycaprolactone.”

“More preferably for medical devices which undergo mechanical challenges, e.g. expansion and contraction, the polymeric materials should be selected from elastomeric polymers such as silicones (e.g. polysiloxanes and substituted polysiloxanes), polyurethanes, thermoplastic elastomers, ethylene vinyl acetate copolymers, polyolefin elastomers, and EPDM rubbers. Because of the elastic nature of these polymers, the coating composition adheres better to the surface of the medical device when the device is subjected to forces, stress or mechanical challenge.”

“The amount of the polymeric material present in the coatings can vary based on the application for the medical device. One skilled in the art is aware of how to determine the desired amount and type of polymeric material used in the coating. For example, the polymeric material in the first coating layer may be the same as or different than the polymeric material in the second coating layer. The thickness of the coating is not limited, but generally ranges from about 25 μm to about 0.5 mm. Preferably, the thickness is about 30 μm to 100 μm.”

Paragraphs 84 through 92 of published U.S. patent application 2004/0030379 describes certain energy sources which may be used in conjunction with the medical devices of this invention. These paragraphs are presented below in their entireties.

“5. Electromagnetic Sources . . . An external electromagnetic source or field may be applied to the patient having an implanted coated medical device using any method known to skilled artisan. In the method of the present invention, the electromagnetic field is oscillated. Examples of devices which can be used for applying an electromagnetic field include a magnetic resonance imaging (“MRI”) apparatus. Generally, the magnetic field strength suitable is within the range of about 0.50 to about 5 Tesla (Webber per square meter). The duration of the application may be determined based on various factors including the strength of the magnetic field, the magnetic substance contained in the magnetic particles, the size of the particles, the material and thickness of the coating, the location of the particles within the coating, and desired releasing rate of the biologically active material.”

“In an MRI system, an electromagnetic field is uniformly applied to an object under inspection. At the same time, a gradient magnetic field, superposing the electromagnetic field, is applied to the same. With the application of these electromagnetic fields, the object is applied with a selective excitation pulse of an electromagnetic wave with a resonance frequency which corresponds to the electromagnetic field of a specific atomic nucleus. As a result, a magnetic resonance (MR) is selectively excited. A signal generated is detected as an MR signal. See U.S. Pat. No. 4,115,730 to Mansfield, U.S. Pat. No. 4,297,637 to Crooks et al., and U.S. Pat. No. 4,845,430 to Nakagayashi. For the present invention, among the functions of the MRI apparatus, the function to create an electromagnetic field is useful for the present invention. The implanted medical device of the present can be located as usually done for MRI imaging, and then an electromagnetic field is created by the MRI apparatus to facilitate release of the biologically active material. The duration of the procedure depends on many factors, including the desired releasing rate and the location of the inserted medical device. One skilled in the art can determine the proper cycle of the electromagnetic field, proper intensity of the electromagnetic field, and time to be applied in each specific case based on experiments using an animal as a model.

In addition, one skilled in the art can determine the excitation source frequency of the elecromagnetic energy source. For example, the electromagnetic field can have an excitation source frequency in the range of about 1 Hertz to about 300 kiloHertz. Also, the shape of the frequency can be of different types. For example, the frequency can be in the form of a square pulse, ramp, sawtooth, sine, triangle, or complex. Also, each form can have a varying duty cycle.”

“b 6. Mechanical Vibrational Energy Source . . . . The mechanical vibrational energy source includes various sources which cause vibration such as ultrasound energy. Examples of suitable ultrasound energy are disclosed in U.S. Pat. No. 6,001,069 to Tachibana et al. and U.S. Pat. No. 5,725,494 to Brisken, PCT publications WO00/16704, WO00/18468, WO00/00095, WO00/07508 and WO99/33391, which are all incorporated herein by reference. Strength and duration of the mechanical vibrational energy of the application may be determined based on various factors including the biologically active material contained in the coating, the thickness of the coating, structure of the coating and desired releasing rate of the biologically active material.”

“Various methods and devices may be used in connection with the present invention. For example, U.S. Pat. No. 5,895,356 discloses a probe for transurethrally applying focused ultrasound energy to produce hyperthermal and thermotherapeutic effect in diseased tissue. U.S. Pat. No. 5,873,828 discloses a device having an ultrasonic vibrator with either a microwave or radio frequency probe. U.S. Pat. No. 6,056,735 discloses an ultrasonic treating device having a probe connected to a ultrasonic transducer and a holding means to clamp a tissue. Any of those methods and devices can be adapted for use in the method of the present invention.”

“Ultrasound energy application can be conducted percutaneously through small skin incisions. An ultrasonic vibrator or probe can be inserted into a subject's body through a body lumen, such as blood vessels, bronchus, urethral tract, digestive tract, and vagina. However, an ultrasound probe can be appropriately modified, as known in the art, for subcutaneous application. The probe can be positioned closely to an outer surface of the patient body proximal to the inserted medical device.”

“The duration of the procedure depends on many factors, including the desired releasing rate and the location of the inserted medical device. The procedure may be performed in a surgical suite where the patient can be monitored by imaging equipment. Also, a plurality of probes can be used simultaneously. One skilled in the art can determine the proper cycle of the ultrasound, proper intensity of the ultrasound, and time to be applied in each specific case based on experiments using an animal as a model.”

“In addition, one skilled in the art can determine the excitation source frequency of the mechanical vibrational energy source. For example, the mechanical vibrational energy source can have an excitation source frequency in the range of about 1 Hertz to about 300 kiloHertz. Also, the shape of the frequency can be of different types. For example, the frequency can be in the form of a square pulse, ramp, sawtooth, sine, triangle, or complex. Also, each form can have a varying duty cycle.”

Paragraphs 93 through 97 of published U.S. patent application 2004/0030379 describe processes for treating body tissue that may be used in conjunction with the medical device of this invention. These paragraphs are presented below in their entireties.”

“D. Treatment of Body Tissue With the Invention . . . . The present invention provides a method of treatment to reduce or prevent the degree of restenosis or hyperplasia after vascular intervention such as angioplasty, stenting, atherectomy and grafting. All forms of vascular intervention are contemplated by the invention, including, those for treating diseases of the cardiovascular and renal system. Such vascular intervention include, renal angioplasty, percutaneous coronary intervention (PCI), percutaneous transluminal coronary angioplasty (PTCA); carotid percutaneous transluminal angioplasty (PTA); coronary by-pass grafting, angioplasty with stent implantation, peripheral percutaneous transluminal intervention of the iliac, femoral or popliteal arteries, carotid and cranial vessels, surgical intervention using impregnated artificial grafts and the like. Furthermore, the system described in the present invention can be used for treating vessel walls, portal and hepatic veins, esophagus, intestine, ureters, urethra, intracerebrally, lumen, conduits, channels, canals, vessels, cavities, bile ducts, or any other duct or passageway in the human body, either in-born, built in or artificially made. It is understood that the present invention has application for both human and veterinary use.”

“The present invention also provides a method of treatment of diseases and disorders involving cell overproliferation, cell migration, and enlargement. Diseases and disorders involving cell overproliferation that can be treated or prevented include but are not limited to malignancies, premalignant conditions (e.g., hyperplasia, metaplasia, dysplasia), benign tumors, hyperproliferative disorders, benign dysproliferative disorders, etc. that may or may not result from medical intervention. For a review of such disorders, see Fishman et al., 1985, Medicine, 2d Ed., J. B. Lippincott Co., Philadelphia.”

“Whether a particular treatment of the invention is effective to treat restenosis or hyperplasia of a body lumen can be determined by any method known in the art, for example but not limited to, those methods described in this section. The safety and efficiency of the proposed method of treatment of a body lumen may be tested in the course of systematic medical and biological assays on animals, toxicological analyses for acute and systemic toxicity, histological studies and functional examinations, and clinical evaluation of patients having a variety of indications for restenosis or hyperplasia in a body lumen.”

“The efficacy of the method of the present invention may be tested in appropriate animal models, and in human clinical trials, by any method known in the art. For example, the animal or human subject may be evaluated for any indicator of restenosis or hyperplasia in a body lumen that the method of the present invention is intended to treat. The efficacy of the method of the present invention for treatment of restenosis or hyperplasia can be assessed by measuring the size of a body lumen in the animal model or human subject at suitable time intervals before, during, or after treatment. Any change or absence of change in the size of the body lumen can be identified and correlated with the effect of the treatment on the subject. The size of the body lumen can be determined by any method known in the art, for example, but not limited to, angiography, ultrasound, fluoroscopy, magnetic resonance imaging, optical coherence tumography and histology.”

The description contained herein is for purposes of illustration and not for purposes of limitation. Changes and modifications may be made to the embodiments of the description and still be within the scope of the invention. Furthermore, obvious changes, modifications or variations will occur to those skilled in the art. Also, all references cited above are incorporated herein, in their entirety, for all purposes related to this disclosure.

1. A medical device that is insertable into the body of a patient comprising: (a) a surface; (b) a first coating layer comprising a biologically active material disposed on at least a portion of the surface; and (c) a second coating layer comprising a polymeric material and nanomagnetic material disposed on the first coating layer, wherein the second coating layer is substantially free of the biologically active material, wherein: said nanomagnetic material has a saturation magentization of from about 2 to about 3000 electromagnetic units per cubic centimeter, wherein said nanomagnetic material is comprised of nanomagnetic particles with an average particle size of less than about 100 nanometers, and wherein the average coherence length between adjacent nanomagnetic particles is less than 100 nanometers.

2. The medical device as recited in claim 1, wherein said nanomagnetic material has an average particle size of less than about 20 nanometers and a phase transition temperature of less than about 200 degrees Celsius.

3. The medical device as recited in claim 1, wherein said medical device further comprises a cytotoxic radioactive material.

4. The medical device as recited in claim 1, wherein said medical device is comprised of a material that is absorbable in living tissue.

5. The medical device as recited in claim 4, wherein said material that is absorbable in living tissue is selected from the group consisting of polyester amides from glycolic acids, polyester amides from lactic acids, polymers and copolymers of gylcolate, polymers and copolymers of lactate, and polydioxanone.

6. The medical device as recited in claim 1, wherein the average particle size of such nanomagnetic particles is less than about 15 nanometers.

7. The medical device as recited in claim 1, wherein said nanomagentic material has a saturation magnetization of at least 2,000 electromagnetic units per cubic centimeter.

8. The medical device as recited in claim 1, wherein said nanomagnetic material has a saturation magnetization of at least 2,500 electromagnetic units per cubic centimeter.

9. The medical device as recited in claim 1, wherein said particles of said nanomagnetic material have a squareness of from about 0.05 to about 1.0.

10. The medical device as recited in claim 1, wherein said particles of said nanomagnetic material are at least triatomic, being comprised of a first distinct atom, a second distinct atom, and a third distinct atom.

11. The medical device as recited in claim 10, wherein said first distinct atom is an atom selected from the group consisting of atoms of actinium, americium, berkelium, californium, cerium, chromium, cobalt, curium, dysprosium, einsteinium, erbium, europium, fermium, gadolinium, holmium, iron, lanthanum, lawrencium, lutetium, manganese, mendelevium, nickel, neodymium, neptunium, nobelium, plutonium, praseodymium, promethium, protactinium, samarium, terbium, thorium, thulium, uranium, and ytterbium, and mixtures thereof.

12. The medical device as recited in claim 10, wherein said first distinct atom is a cobalt atom.

13. The medical device as recited in claim 11, wherein said particles of nanomagnetic material are comprised of atoms of cobalt and atoms of iron.

14. The medical device as recited in claim 11, wherein said first distinct atom is a radioactive cobalt atom.

15. The medical device as recited in claim 10, wherein said particles of nanomagnetic material are comprised of a said first distinct atom, said second distinct atom, said third distinct atom, and a fourth distinct atom.

16. The medical device as recited in claim 15, wherein said particles of nanomagnetic material are comprised of a fifth distinct atom.

17. The medical device as recited in claim 1, wherein said particles of nanomagnetic material have a squareness of from about 0.1 to about 0.9.

18. The medical device as recited in claim 1, wherein said particles of nanomagnetic material have a squareness is from about 0.2 to about 0.8.

19. The medical device as recited in claim 1, wherein said particles of nanomagnetic material have an average size of less of less than about 3 nanometers.

20. The medical device as recited in claim 1, wherein said particles of nanomagnetic material have an average size of less than about 15 nanometers.

21. The medical device as recited in claim 1, wherein said particles of nanomagnetic material have an average size is less than about 11 nanometers.

22. The medical device as recited in claim 1, wherein said particles of nanomagnetic material have a phase transition temperature of less than 46 degrees Celsius.

23. The medical device as recited in claim 1, wherein said particles of nanomagnetic material have a phase transition temperature of less than about 50 degrees Celsius.

24. The medical device as recited in claim 1, wherein said nanomagnetic material has a coercive force of from about 0.1 to about 10 Oersteds.

25. The medical device as recited in claim 1, wherein said particles of nanomagnetic material have a relative magnetic permeability of from about 1.5 to about 2,000.

26. The medical device as recited in claim 1, wherein said particles of nanomagnetic material have a saturation magnetization of at least 100 electromagnetic units per cubic centimeter.

27. The medical device as recited in claim 1, wherein said particles of nanomagnetic material have a saturation magnetization of at least about 200 electromagnetic units (emu) per cubic centimeter.

28. The medical device as recited in claim 1, wherein said particles of nanomagnetic material have a saturation magnetization of at least about 1,000 electromagnetic units per cubic centimeter.

29. The medical device as recited in claim 1, wherein said particles of nanomagnetic material have a coercive force of from about 0.01 to about 5,000 Oersteds.

30. The medical device as recited in claim 1, wherein said particles of nanomagnetic material have a coercive force of from about 0.01 to about 3,000 Oersteds.

31. The medical device as recited in claim 1, wherein said particles of nanomagnetic material are disposed within a film that has a heat shielding factor of at least 0.2.

32. The medical device as recited in claim 1, wherein said particles of nanomagnetic material have a relative magnetic permeability of from about 1 to about 500,000.

33. The medical device as recited in claim 1, wherein said particles of nanomagnetic material have a relative magnetic permeability of from about 1.5 to about 260,000.

34. The medical device as recited in claim 1, wherein said particles of nanomagnetic material have a mass density of at least about 0.001 grams per cubic centimeter.

35. The medical device as recited in claim 1, wherein said particles of nanomagnetic material have a mass density of at least about 1 gram per cubic centimeter.

36. The medical device as recited in claim 1, wherein said particles of nanomagnetic material have a mass density of at least about 3 grams per cubic centimeter.

37. The medical device as recited in claim 1 wherein said particles of nanomagnetic material have a mass density of at least about 4 grams per cubic centimeter.

38. The medical device as recited in claim 15, wherein said second distinct atom has a relative magnetic permeability of about 1.0.

39. The medical device as recited in claim 38, wherein said second distinct atom is an atom selected from the group consisting of aluminum, antimony, barium, beryllium, boron, bismuth, calcium, gallium, germanium, gold, indium, lead, magnesium, palladium, platinum, silicon, silver, strontium, tantalum, tin, titanium, tungsten, yttrium, zirconium, magnesium, and zinc.

40. The medical device as recited in claim 39, wherein said third distinct atom is an atom selected from the group consisting of argon, bromine, carbon, chlorine, fluorine, helium, hydrogen, iodine, krypton, oxygen, neon, nitrogen, phosphorus, sulfur, and xenon.

41. The medical device as recited in claim 40, wherein said third distinct atom is nitrogen.

42. The medical device as recited in claim 41, wherein said nanomagnetic particles are represented by the formula AₓB_{y}C_{z}, wherein A is said first distinct atom, B is said second distinct atom, C is said third distinct atom, and x+y+z is equal to 1.

43. The medical device as recited in claim 42, wherein said nanomagnetic particles are comprised of atoms of oxygen.

44. The medical device as recited in claim 43, wherein said nanomagnetic particles are comprised of atoms of iron.

45. The medical device as recited in claim 44, wherein said atoms of iron are atoms of radioactive iron.

46. The medical device as recited in claim 44, wherein nanomagnetic particles are comprised of atoms of cobalt.

47. The medical device as recited in claim 46, wherein said atoms of cobalt are atoms of radioactive cobalt.

48. The medical device as recited in claim 1, wherein said particles of nanomagnetic material are present in the form of a coating with a thickness of from about 400 to about 2000 nanometers.

49. The medical device as recited in claim 48, wherein said coating has a thickness of from about 600 to about 1200 nanometers.

50. The medical device as recited in claim 49, wherein said coating has a morphological density of at least about 98 percent.

51. The medical device as recited in claim 50, wherein said coating has a morphological density of at least about 99 percent.

52. The medical device as recited in claim 51, wherein said coating has a morphological density of at least about 99.5 percent.

53. The medical device as recited in claim 49, wherein said coating has an average surface roughness of less than about 100 nanometers.

54. The medical device as recited in claim 49, wherein said coating has an average surface roughness of less than about 10 nanometers.

55. The medical device as recited in claim 1, wherein said coating is biocompatible.

56. The medical device as recited in claim 1, wherein said coating is hydrophobic.

57. The medical device as recited in claim 1, wherein said coating is hydrophilic.

58. The medical device of claim 1, wherein the first coating layer is substantially free of said magnetic particles.

59. The medical device as recited in claim 1 wherein the first coating layer further comprises a polymeric material.

60. The medical device as recited in claim 55, wherein the polymeric material in the first coating layer is different than the polymeric material in the second coating layer.

61. The medical device as recited in claim 1, further comprising a sealing layer disposed on the second coating layers wherein the sealing layer comprises a polymeric material and is substantially free of the biologically active material and the magnetic particles.

62. The medical device as recited in claim 1, wherein the medical device is a stent having a sidewall comprising a plurality of struts, and wherein the surface is a part of the struts.
